(19) United States
(12) Patent Application Publication
(10) Pub. No.: US 2014/0200215 A1
(43) Pub. Date: Jul. 17, 2014

(54) LYSOPHOSPHATIDIC ACID RECEPTOR ANTAGONISTS

(71) Applicant: INTERMUNE, INC., Brisbane, CA (US)

(72) Inventors: Brad Owen Buckman, Oakland, CA (US); John Beamond Nicholas, Redwood City, CA (US); Kumaraswamy Emayan, Berkeley, CA (US); Scott D. Seiwert, Half Moon Bay, CA (US)

(21) Appl. No.: 13/795,605

(22) Filed: Mar. 12, 2013

Related U.S. Application Data
(60) Provisional application No. 61/752,884, filed on Jan. 15, 2013.
(60) Provisional application No. 61/764,487, filed on Feb. 13, 2013.

(51) Int. Cl. C07D 261/14; C07D 487/04; C07D 417/10; C07D 495/04; C07D 275/03; C07D 413/10

(52) U.S. Cl. CPC C07D 261/14; C07D 275/03; C07D 413/10; C07D 417/10; C07D 495/04; C07D 487/04
USPC 514/233.8; 514/380; 548/372.5; 514/372; 548/214; 548/364.7; 548/180; 514/367; 514/312; 546/156; 514/407; 548/364.4; 544/140; 544/350; 514/249; 546/114; 514/301; 544/236; 514/248; 435/375

(57) ABSTRACT

Compounds, methods of making such compounds, pharmaceutical compositions and medicaments comprising such compounds, and methods of using such compounds to treat, prevent or diagnose diseases, disorders, or conditions associated with one or more of the lysophosphatidic acid receptors are provided.

### INCORPORATION BY REFERENCE TO RELATED APPLICATIONS

The present application claims the benefit of priority to U.S. Appl. No. 61/752,884, filed Jan. 15, 2013 and U.S. Appl. No. 61/764,487, filed Feb. 13, 2013, both of which are hereby incorporated by references in their entireties. Any and all priority claims identified in the Application Data Sheet, or any correction thereto, are hereby incorporated by reference under 37 CFR 1.57.

### FIELD

Compounds, methods of making such compounds, pharmaceutical compositions and medicaments comprising such compounds, and methods of using such compounds to treat, prevent or diagnose diseases, disorders, or conditions associated with one or more of the lysophosphatidic acid receptors are provided.

### BACKGROUND

Lysophospholipids are membrane-derived bioactive lipid mediators that affect fundamental cellular functions. These cellular functions include, but are not limited to, proliferation, differentiation, survival, migration, adhesion, invasion, and morphogenesis. These cellular functions influence biological processes that include, but are not limited to, neurogenesis, angiogenesis, wound healing, fibrosis, immunity, and carcinogenesis.

Lysophosphatidic acid (LPA) is a lysophospholipid that has been demonstrated to act through sets of specific G protein-coupled receptors (GPCRs) in an autocrine and paracrine fashion. LPA binding to its cognate GPCRs (LPA₁, LPA₂, LPA₃, LPA₄, LPA₅, and LPA₆) activates intracellular signaling pathways to produce a variety of biological responses. Antagonists of the LPA receptors can be employed in the treatment of diseases, disorders, or conditions in which LPA plays a role.

### SUMMARY

Some embodiments disclosed herein include a compound having the structure of Formula (I):

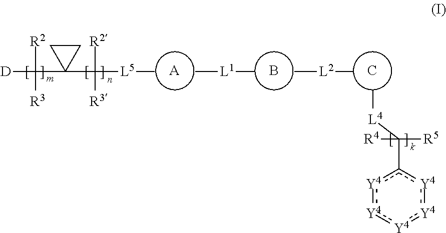

or a pharmaceutically acceptable salt thereof, wherein:

A is an acetylene and B is a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, haloalkyl, halogen, hydroxy, alkoxy, haloalkoxy, cyano, or oxo;

or alternatively,

B is an acetylene and A is a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, haloalkyl, halogen, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

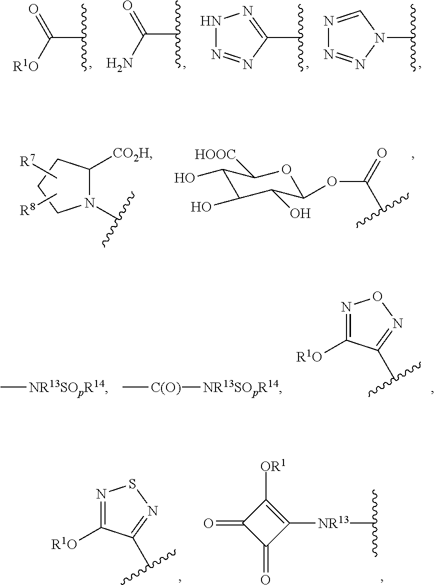

or carboxylic acid isosteres;

L⁴ is

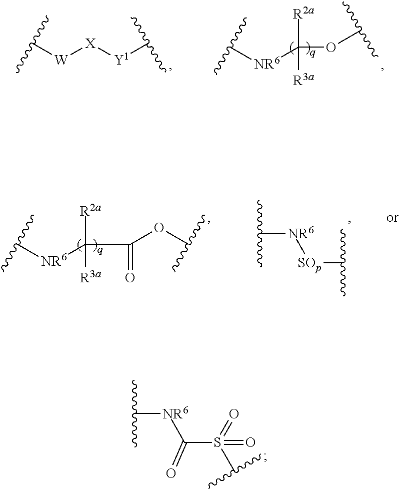

or alternatively,

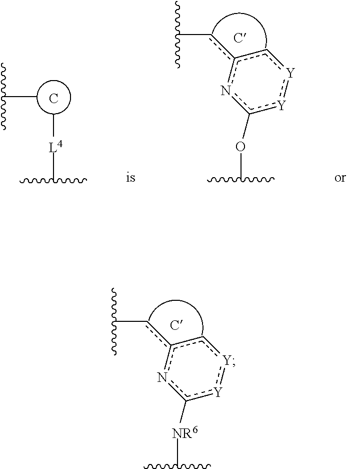

wherein

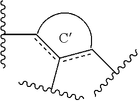

is selected from:

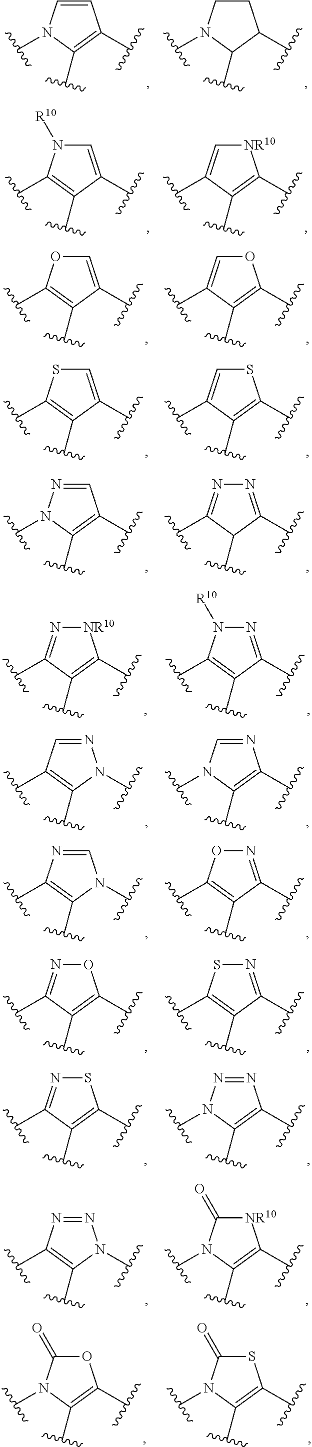

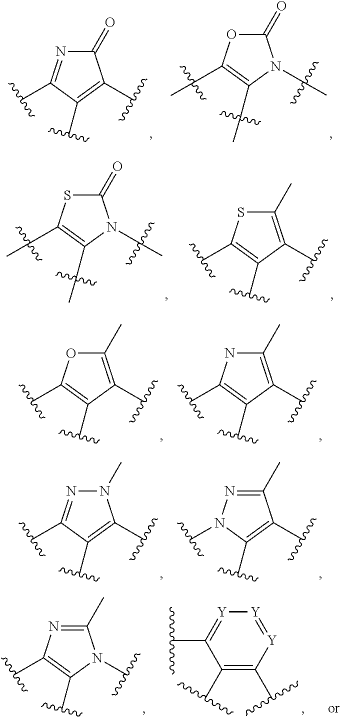

optionally substituted variants thereof;

L¹ is selected from selected from a single bond, a —O— linker, a

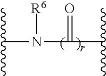

linker, a —C(O)— linker, a —CH₂— linker, a —CH₂O— linker, a —C≡C— linker, or a —CH═CH— linker;

L² is selected from a single bond, a —O— linker, a

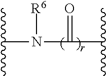

linker, a —C(O)— linker, a —CH₂— linker, a —CH₂O— linker, a —C≡C— linker, or a —CH═CH— linker;

L⁵ is selected from a single bond, a —CH₂O— linker, a —CH═CH— linker, a —C≡C— linker, a

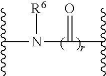

linker, or a 4-7 membered heterocyclyl;

W is C(R⁶)₂, NR⁶, or O;

X is —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R², R³, R^{2′}, and R^{3′} are each independently selected from hydrogen, alkyl, aryl, or heteroaryl; or R² and R³ are joined together with the atom to which they are attached to form an optionally substituted cycloalkyl or an optionally substituted heterocyclyl; or R^{2′} and R^{3′} are joined together with the atom to which they are attached to form an optionally substituted cycloalkyl or an optionally substituted heterocyclyl;

or R^{2′} is selected from hydrogen, alkyl, aryl, or heteroaryl and R^{3′} is joined to an atom alpha to a point of attachment of L⁵ to A to form an optionally substituted cycloalkyl or an optionally substituted heterocyclyl; or R^{3′} is selected from hydrogen, alkyl, aryl or heteroaryl and R^{2′} is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3; provided that the total of m+n is equal to or larger than 1;

k is an integer from 0-3;

p is an integer from 1-2;

q is an integer from 1-6;

r is an integer of 0 or 1, and

[symbol] represents a single or double bond.

Some embodiments disclosed herein include a compound having the structure of Formula (II):

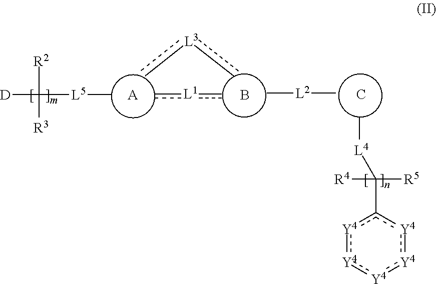

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, halogen, haloalkyl, hydroxy, alkoxy, haloalkoxy, cyano, or oxo;

B is an acetylene or a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, halogen, haloalkyl, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

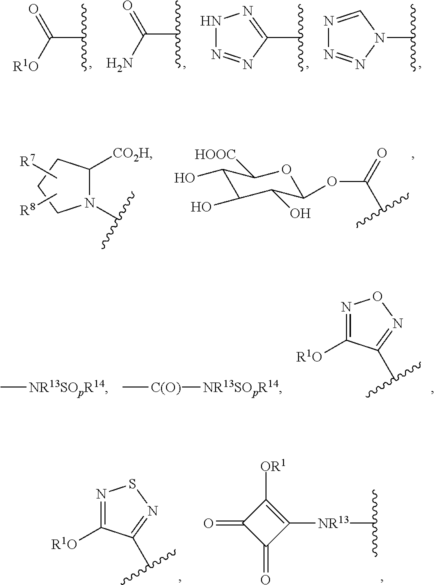

or carboxylic acid isosteres;

L⁴ is

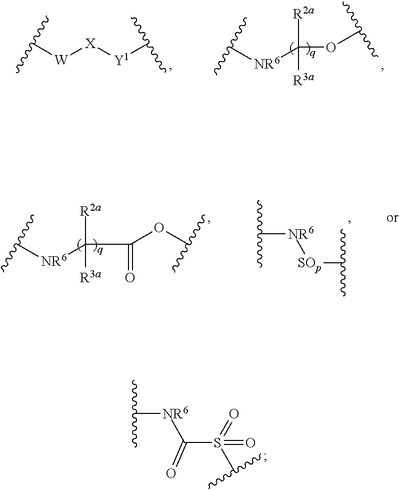

or alternatively,

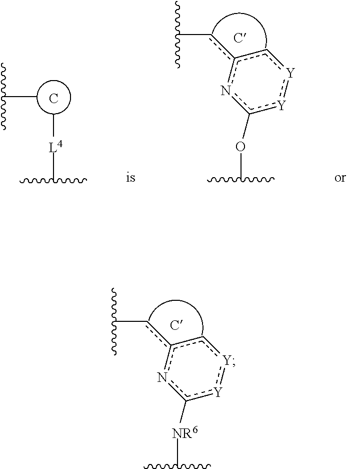

wherein

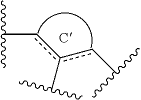

is selected from:

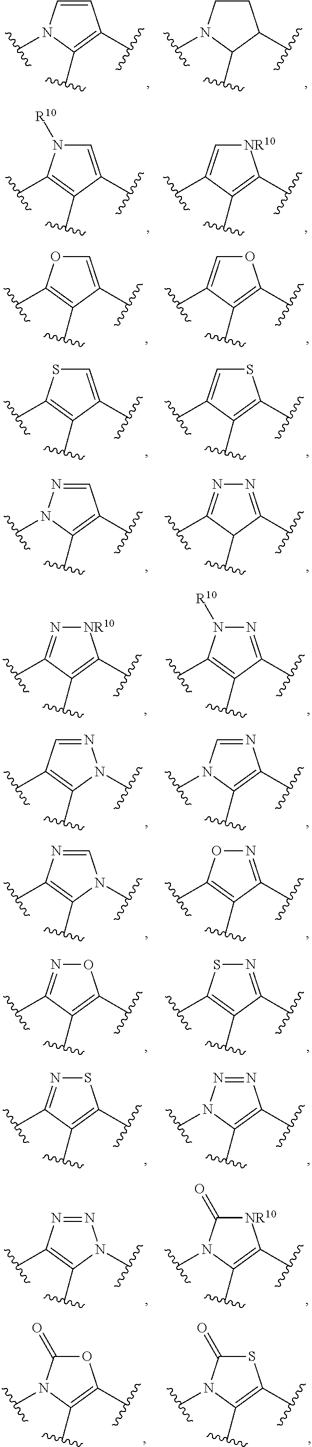

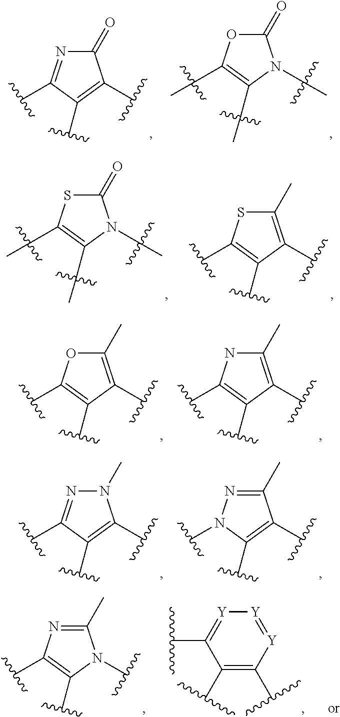

optionally substituted variants thereof;

L¹ is selected from a single bond, a —O— linker, a

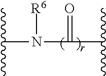

linker, a —C(O)— linker, a —CH₂— linker, a —CH₂O— linker, a —C≡C— linker, a —CH═CH— linker, or a ═C(R¹¹)— linker;

L² is selected from a single bond, a —O— linker, a

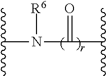

linker, a —C(O)— linker, a —CH₂— linker, a —CH₂O— linker, a —C≡C— linker, or a —CH═CH— linker;

L³ is absent or selected from

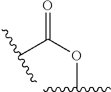 [SMILES: *OC(*)=O]

or a ═C(R¹¹)— linker;

L⁵ is a

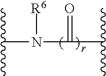

linker or a —C≡C— linker;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen, alkyl optionally substituted with one or more substituents selected from the group consisting of halogen, hydroxy and alkoxy; halogen; haloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

k is an integer from 0-3;

p is an integer from 1-2;

q is an integer from 1-6;

r is an integer of 0 or 1, and

[symbol] represents a single or double bond.

Some embodiments disclosed herein include a compound having the structure of Formula (III):

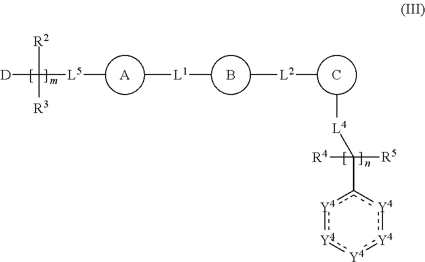

or a pharmaceutically acceptable salt thereof, wherein:

A is selected from

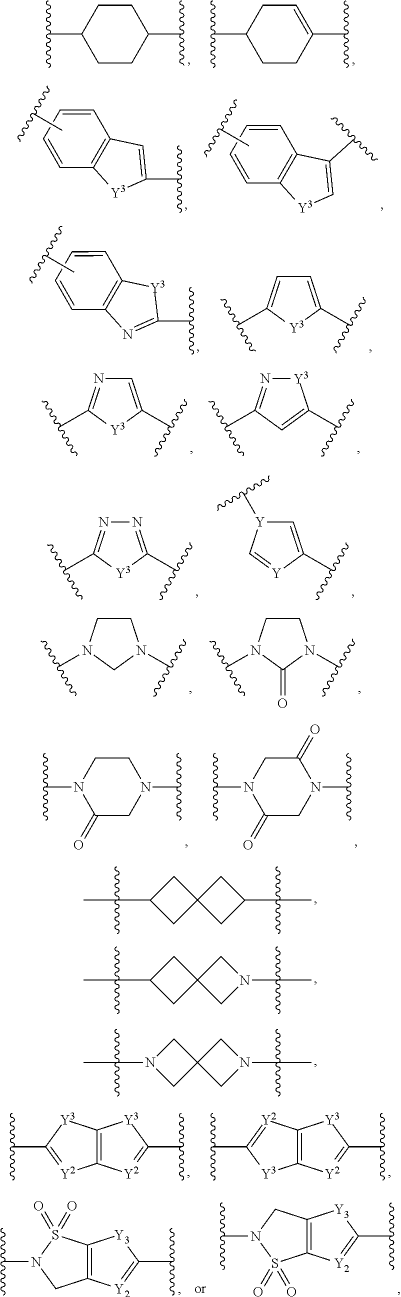

wherein A is unsubstituted or substituted with one or more substituents selected from alkyl, halogen, haloalkyl, cyano, hydroxy, alkoxy, haloalkoxy, or oxo; and

B is a ring system selected from the group consisting of 6-11 membered aryl, 5-11 membered heteroaryl, 4-11 membered heterocyclyl, and 4-11 membered carbocyclyl, and wherein B is unsubstituted or substituted with one or more substituents selected from alkyl, halogen, hydroxy, alkoxy, haloalkoxy, cyano, or oxo;

or alternatively,

B is selected from

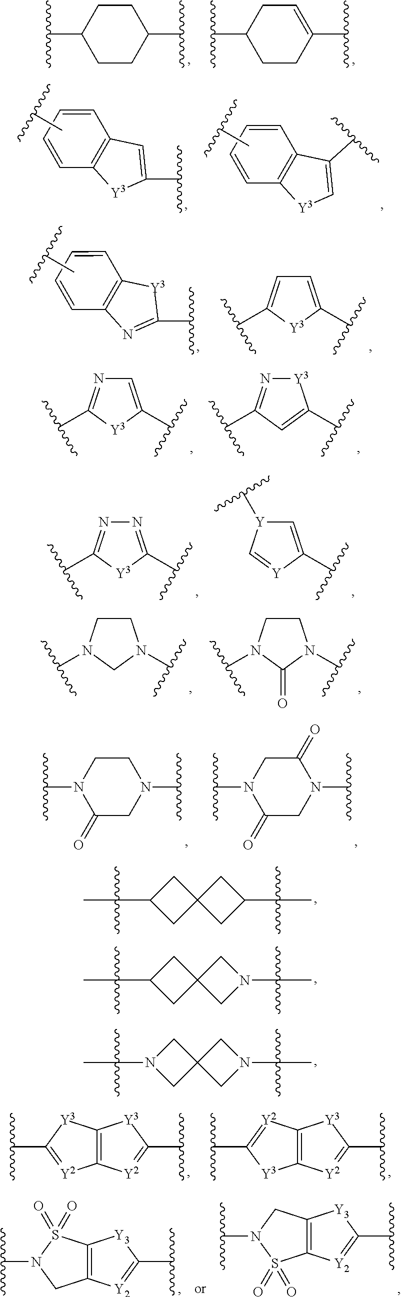

wherein B is unsubstituted or substituted with one or more substituents selected from alkyl, halogen, haloalkyl, cyano, hydroxy, alkoxy, haloalkoxy, or oxo; and A is a ring system selected from the group consisting of 6-11 membered aryl, 5-11 membered heteroaryl, 4-11 membered heterocyclyl, and 4-11 membered carbocyclyl, and wherein A is unsubstituted or substituted with one or more substituents selected from alkyl, halogen, haloalkyl, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

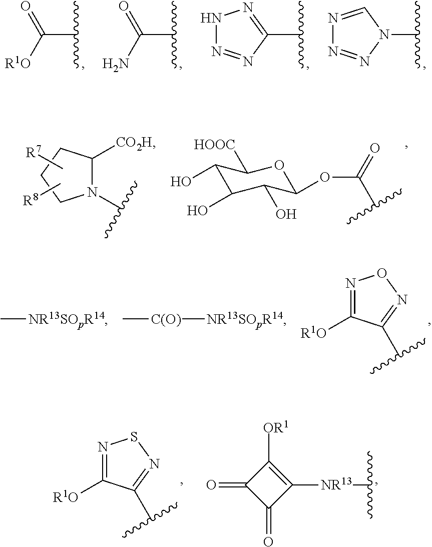

or carboxylic acid isosteres;

L⁴ is

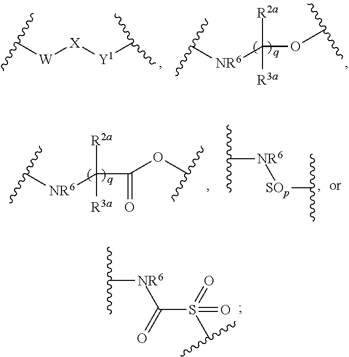

or alternatively,

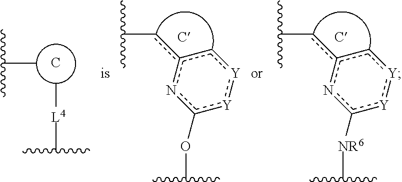

wherein

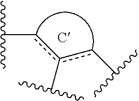

is selected from:

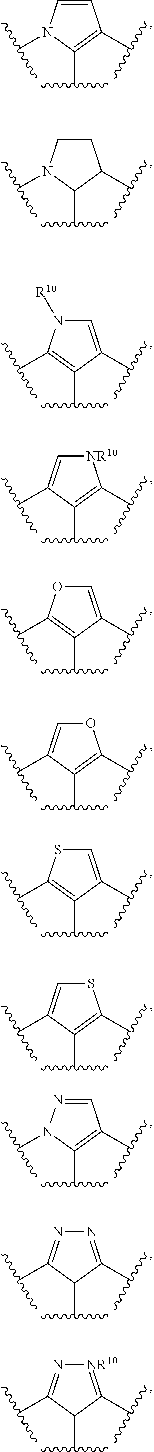

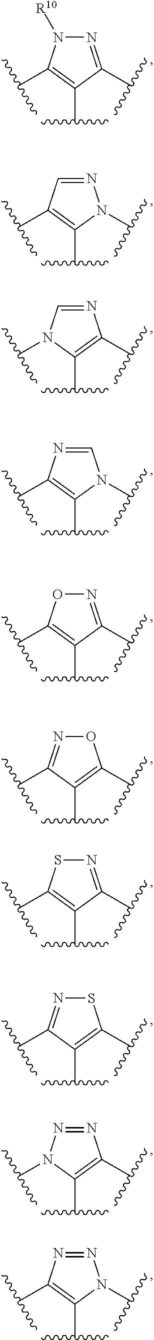

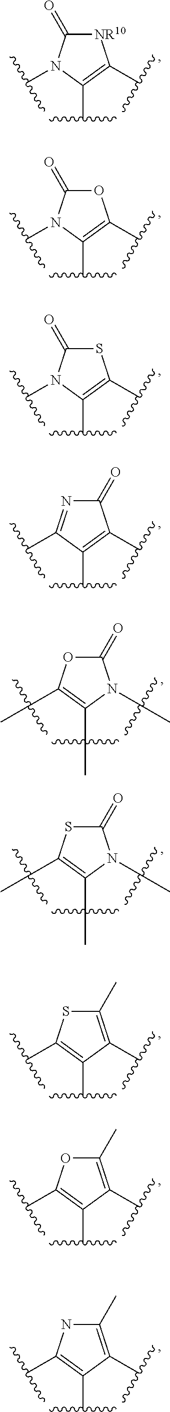

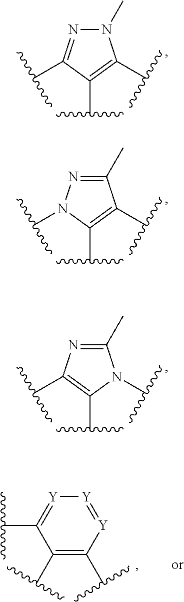

optionally substituted variants thereof;

L¹ is selected from a single bond, a —O— linker, a —C(O)— linker, a —CH₂O— linker, a

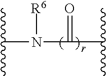

linker, a —C≡C— linker, or a —CH═CH— linker;

L² is selected from a single bond, a —O— linker, a

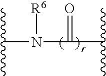

linker, a —C(O)— linker, a —CH₂— linker, a —CH₂O— linker, a —C≡C— linker, or a —CH═CH— linker;

L⁵ is selected from a single bond, a —CH₂O— linker, a —CH═CH— linker, a —C≡C— linker,

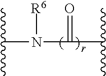

, or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

Y² is selected from —CH═ or N;

Y³ is selected from C(R⁶)₂, NR⁶, O, or S;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

r is an integer of 0 or 1, and

[symbol] represents a single or double bond; provided that

when D is —C(O)OR¹; R¹ is hydrogen or alkyl; m is 1; A is cyclohexyl; B is phenyl; L³ is absent; L⁵ is a single bond; L¹ is a single bond;

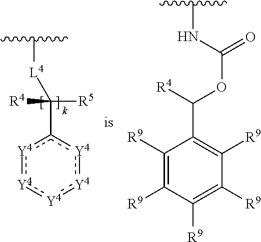

wherein R⁹ is selected from H, alkyl or halogen; then C cannot be a triazole or pyrazole;

when D is —C(O)OR¹; R¹ is hydrogen or alkyl; A is cyclohexyl or

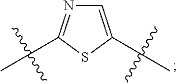 [SMILES: *c1cnc(*)s1]

B is phenyl;

L³ is absent; L⁵ is a single bond; L¹ is a single bond;

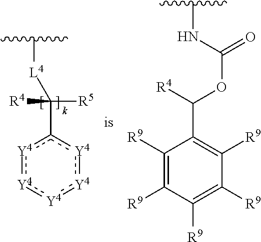

wherein R⁹ is selected from H, alkyl or halogen; and C is isoxazole; then m is not 0; and

when D is —C(O)OR¹; R¹ is hydrogen or alkyl; m is 1; A is phenyl; B is

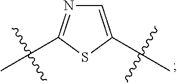 [SMILES: *c1cnc(*)s1]

### L³ is

absent; L⁵ is a single bond; L¹ is a single bond;

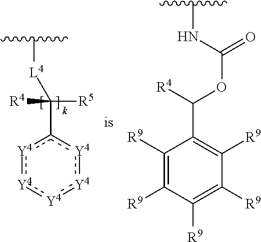

wherein R⁹ is selected from H, alkyl or halogen; then C is not isoxazole.

Some embodiments disclosed herein include a compound having the structure of Formula (IV):

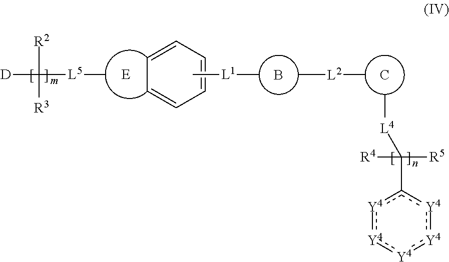

or a pharmaceutically acceptable salt thereof, wherein:

B is an acetylene or a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, haloalkyl, halogen, hydroxy, alkoxy, haloalkoxy, cyano, or oxo;

E is a 5 or 6 membered heterocyclyl comprising one heteroatom selected from oxygen, nitrogen or sulfur, wherein

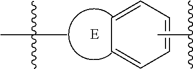

is unsubstituted or substituted with one or more substituents selected from alkyl, halogen, haloalkyl, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

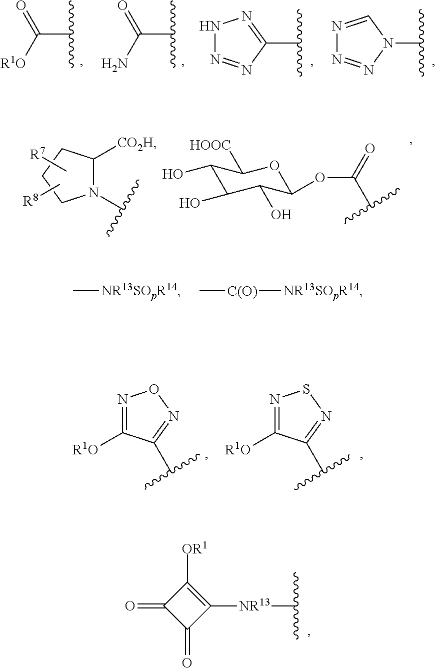

or carboxylic acid isosteres;

L⁴ is

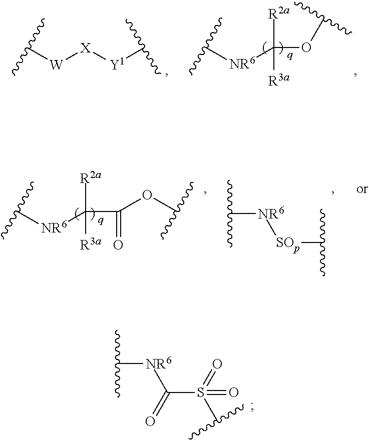

or alternatively,

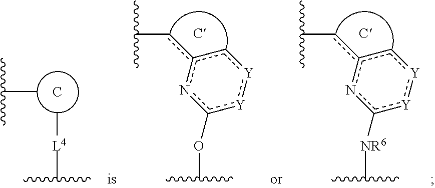

wherein

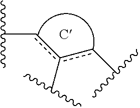

is selected from:

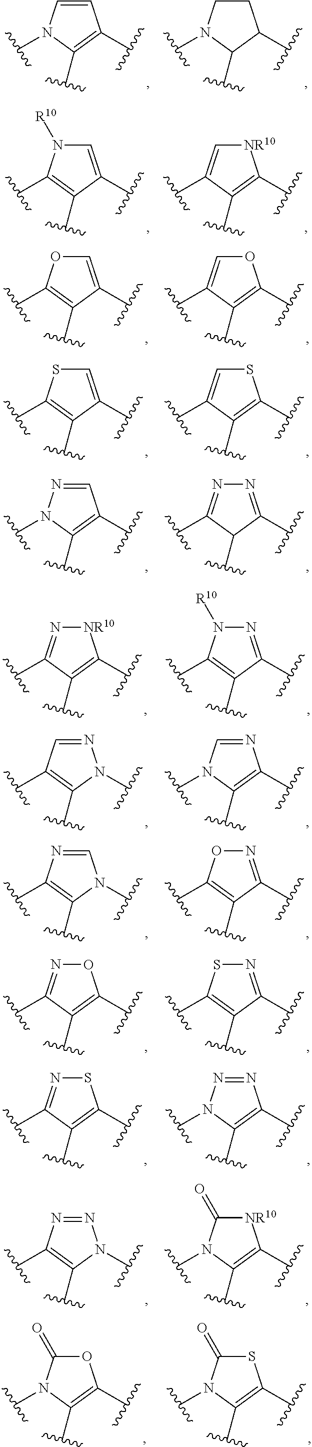

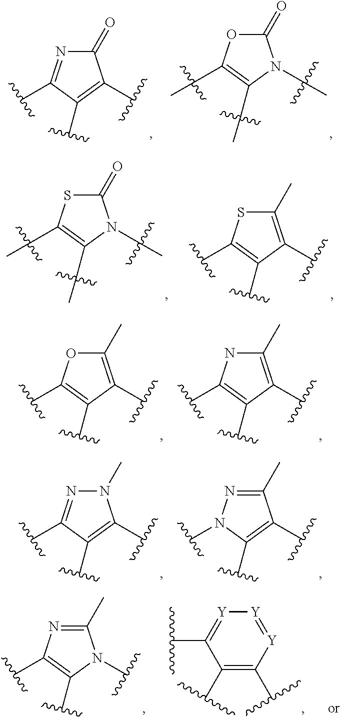

optionally substituted variants thereof;

L¹ is selected from a single bond, a —O— linker, a

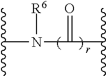

linker, a —C(O)— linker, a —CH₂— linker, a —CH₂O— linker, a —C≡C— linker, or a —CH═CH— linker;

L² is selected from a single bond, a —O— linker, a

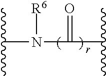

linker, a —C(O)— linker, a —CH₂— linker, a —CH₂O— linker, a —C≡C— linker, or a —CH═CH— linker;

L⁵ is selected from a single bond, a —CH₂O— linker, a —OCH₂— linker, a —CH═CH— linker, a —C≡C— linker, a

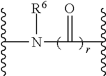

linker or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen, alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

r is an integer of 0 or 1; and

[symbol] represents a single or double bond.

Some embodiments disclosed herein include a compound having the structure of Formula (V):

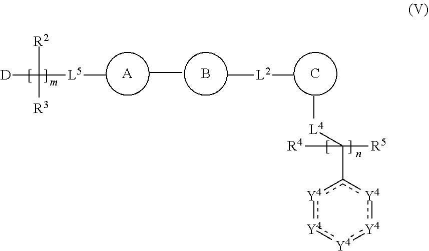

or a pharmaceutically acceptable salt thereof, wherein:

B is an acetylene and A is a ring system selected from

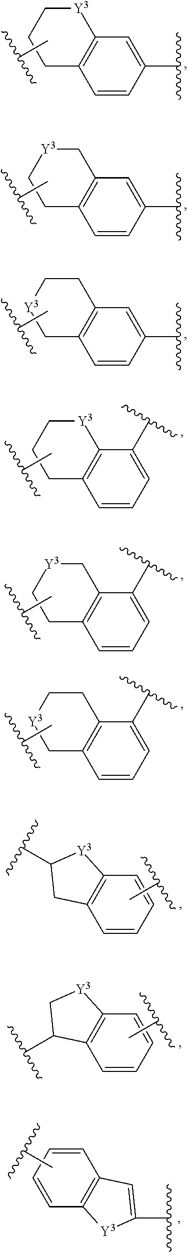

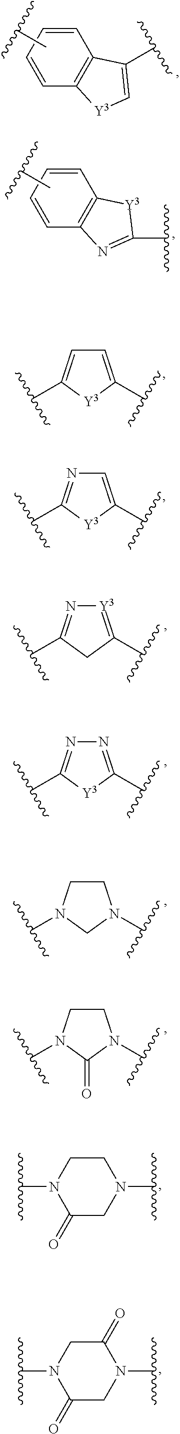

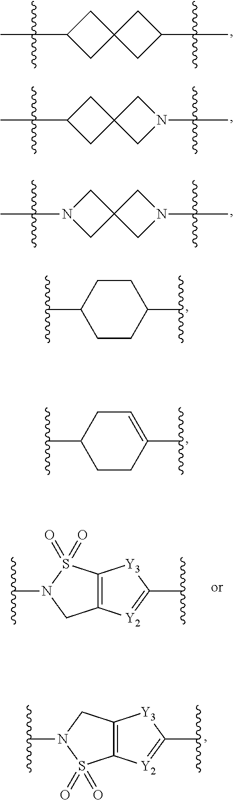

wherein A is unsubstituted or substituted with one or more substituents selected from alkyl, halogen, haloalkyl, hydroxy, alkoxy, haloalkoxy, cyano, or oxo;

or alternatively,

A is an acetylene and B is a ring system selected from

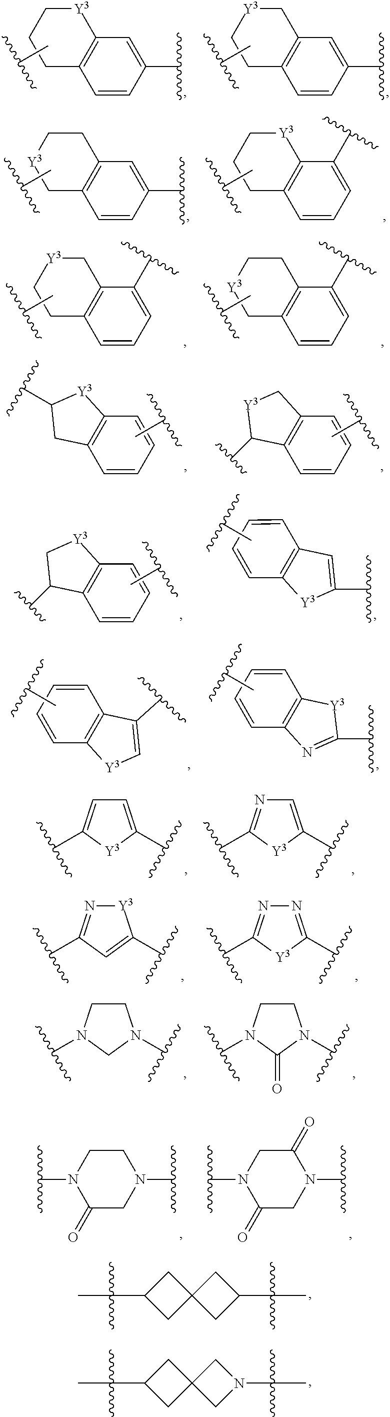

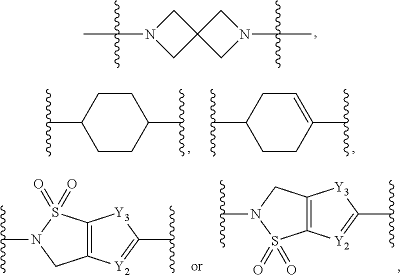

wherein B is unsubstituted or substituted with one or more substituents selected from alkyl, halogen, haloalkyl, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

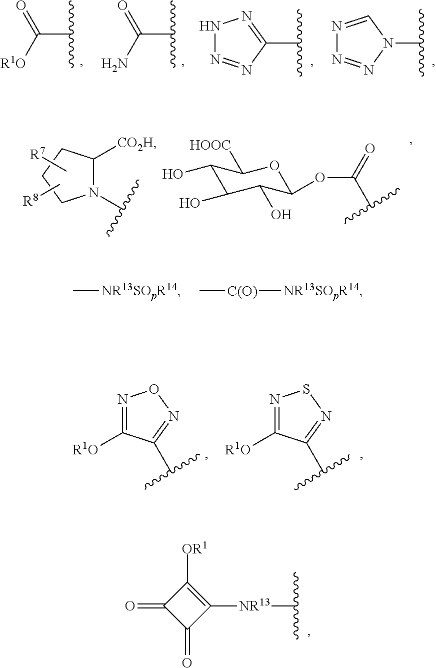

or carboxylic acid isosteres;

L⁴ is

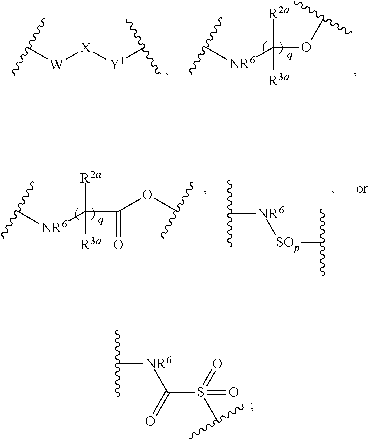

or alternatively,

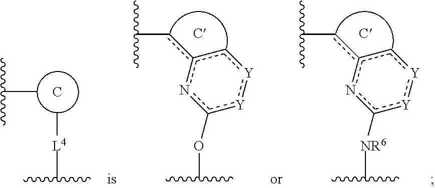

wherein

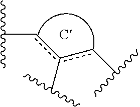

is selected from:

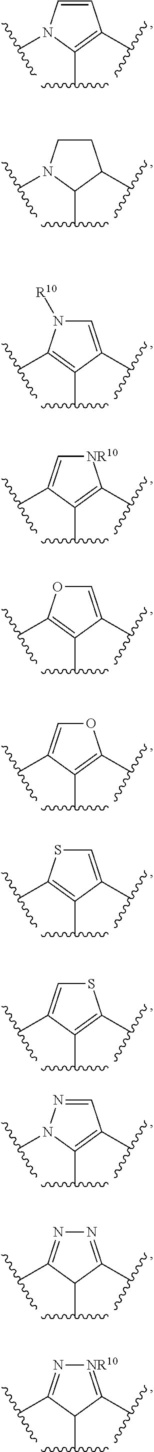

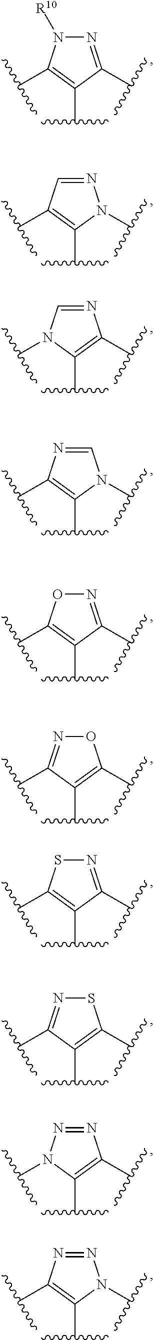

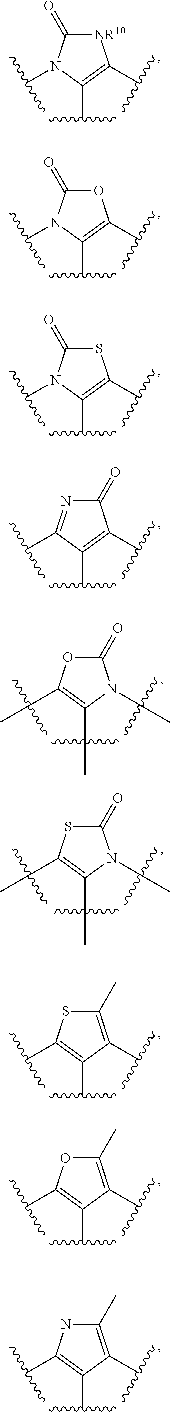

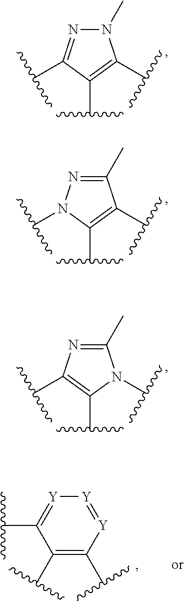

optionally substituted variants thereof;

L² is selected from a single bond, a —O— linker, a

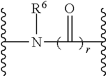

linker, a —C(O)— linker, a —CH₂— linker, a —CH₂O— linker, or a —CH═CH— linker;

L⁵ is selected from a single bond, a —CH₂O— linker, a —CH═CH— linker, a

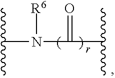

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

Y³ is selected from C(R⁶)₂, NR⁶, O, or S;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

r is an integer of 0 or 1, and

[symbol] represents a single or double bond.

Some embodiments disclosed herein include a compound having the structure of Formula (VI):

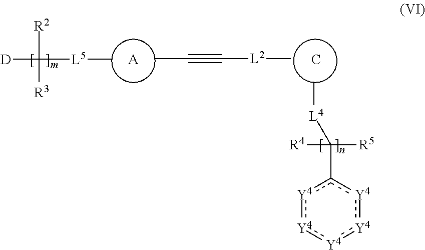

or a pharmaceutically acceptable salt thereof, wherein

A is a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, halogen, haloalkyl, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

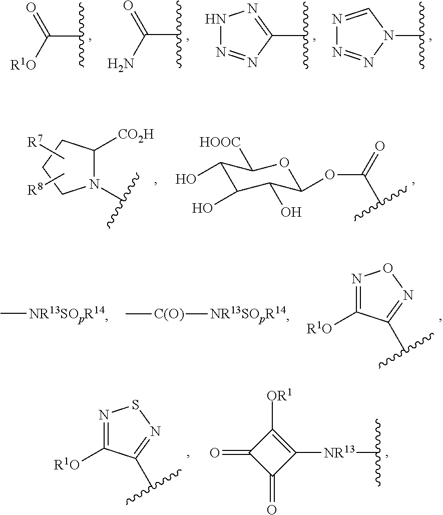

or carboxylic acid isosteres;

L⁴ is

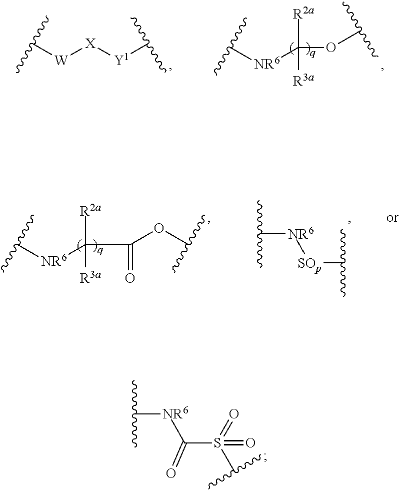

or alternatively,

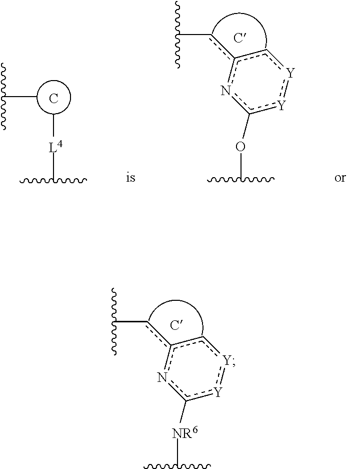

wherein

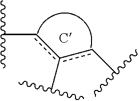

is selected from:

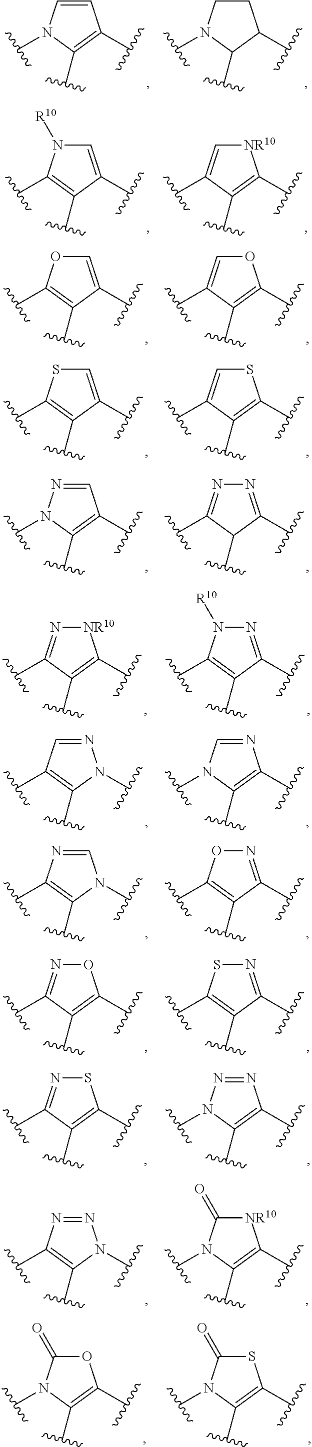

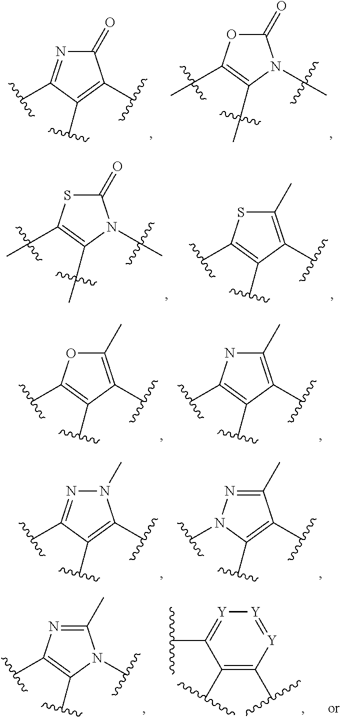

optionally substituted variants thereof;

L² is selected from a single bond, a —O— linker, a

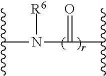

linker, a —C(O)— linker, a —CH₂— linker, a —CH₂O— linker, or a —CH═CH— linker;

L⁵ is selected from a —CH═CH— linker or a —C≡C— linker;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

r is an integer of 0 or 1, and

[symbol] represents a single or double bond.

Some embodiments disclosed herein include a compound having the structure of Formula (VII):

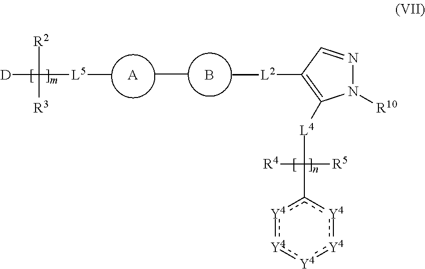

or a pharmaceutically acceptable salt thereof, wherein:

A is an acetylene and B is a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, halogen, haloalkyl, hydroxy, alkoxy, haloalkoxy, cyano, or oxo;

or alternatively,

B is an acetylene, or is absent when L² is —(CH₂)ₖ— linker, and A is a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, halogen, haloalkyl, hydroxy, alkoxy, haloalkoxy, cyano, or oxo; or B is optionally absent when L² is —(CH₂)ₖ— linker;

D is selected from —OH,

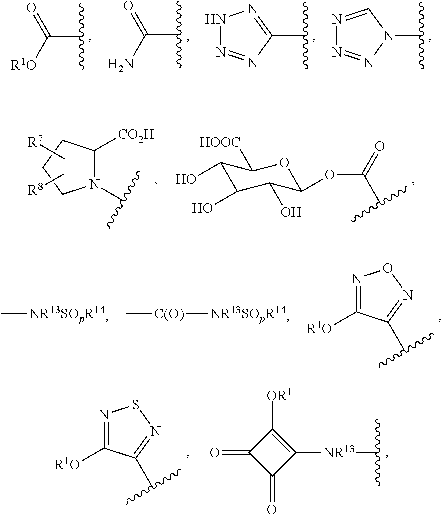

or carboxylic acid isosteres;

L⁴ is

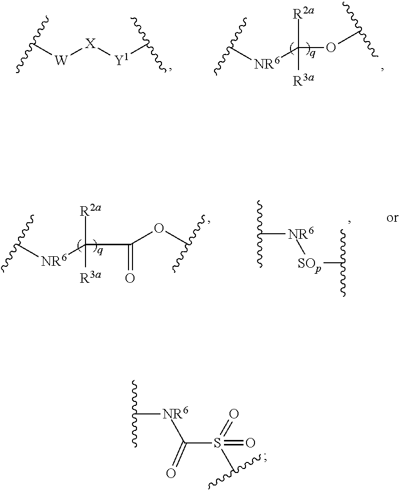

L² is selected from a single bond, a —O— linker, a

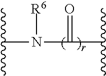

linker, a —C(O)— linker, a —CH₂— linker, a —(CH₂)ₖ— linker, a —CH₂O— linker, a —C≡C— linker, or a —CH═CH— linker;

L⁵ is selected from a single bond, a —CH₂O— linker, a —CH═CH— linker, a —C≡C— linker, a

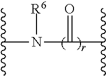

linker, or a 4-7 membered heterocyclyl;

W is selected from C(R⁶)₂, NR⁶, or O;

X is selected from —C(O) or S(O)ₚ;

Y¹ is selected from C(R⁶)₂, NR⁶, or O;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and

R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

k is an integer from 2-4;

p is an integer from 1-2;

q is an integer from 1-6;

r is an integer of 0 or 1, and

[symbol] represents a single or double bond.

Some embodiments disclosed herein include a compound having the structure of Formula (VIII):

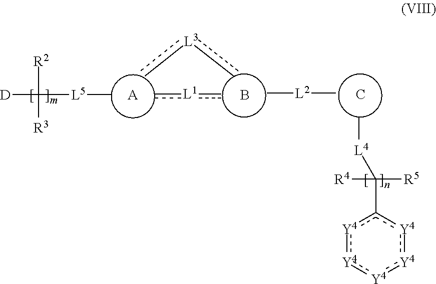

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, halogen, haloalkyl, hydroxy, alkoxy, haloalkoxy, cyano, or oxo;

B is an acetylene or a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, halogen, haloalkyl, hydroxy, alkoxy, haloalkoxy, cyano, or oxo;

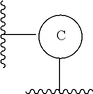

is a ring system selected from the group consisting of

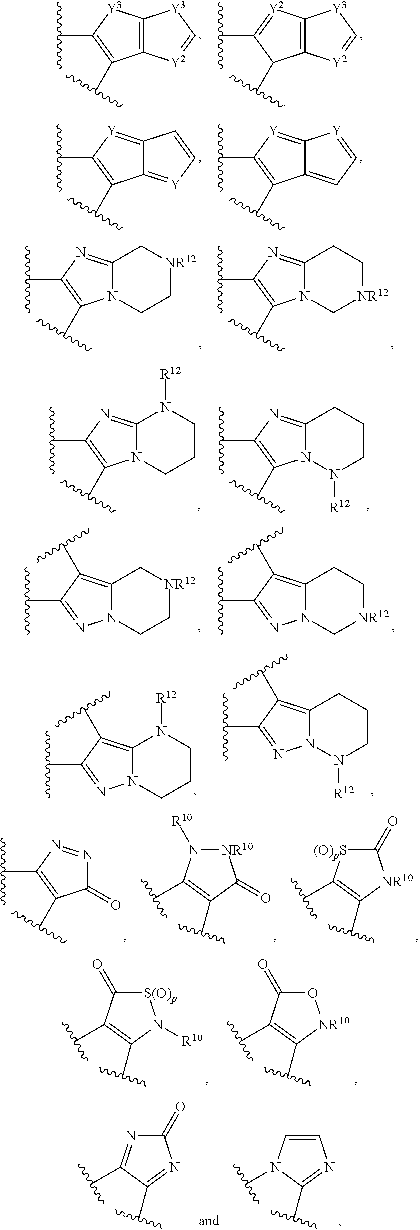

wherein C is optionally substituted;

D is selected from —OH,

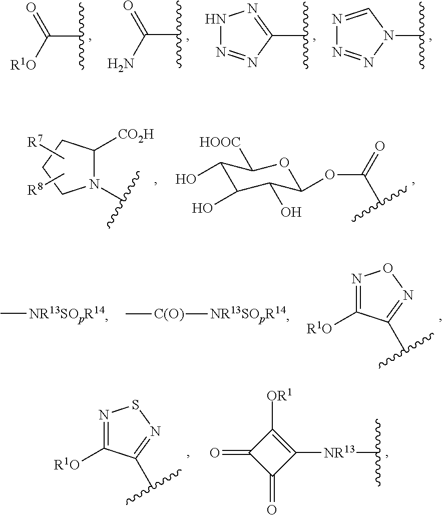

or carboxylic acid isosteres;

L⁴ is

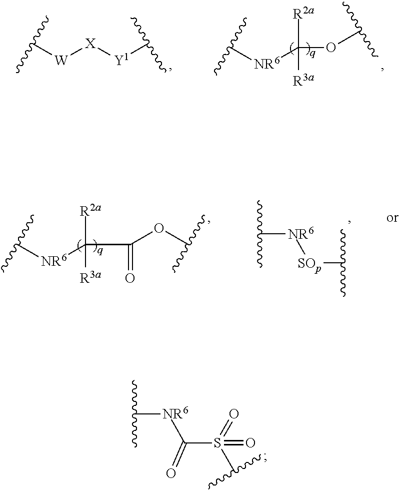

L¹ is selected from a single bond, a —O— linker, a

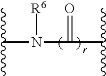

linker, a —C(O)— linker, a —CH₂— linker, a —CH₂O— linker, a —C≡C— linker, a —CH═CH— linker, or a ═C(R¹¹)— linker;

L² is selected from a single bond, a —O— linker, a

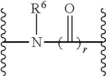

linker, a —C(O)— linker, a —CH₂— linker, a —CH₂O— linker, a —C≡C— linker, or a —CH═CH— linker;

L³ is absent,

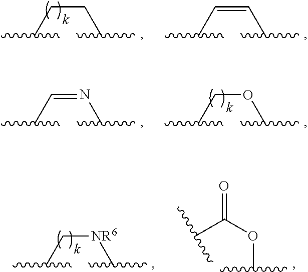

or a ═C(R¹¹)— linker;

L⁵ is selected from a single bond, a —CH₂O— linker, a —CH═CH— linker, a —C≡C— linker, a

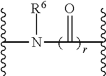

linker, or a 4-7 membered heterocyclyl;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

Y² is selected from —CH═ or N;

Y³ is selected from C(R⁶)₂, NR⁶, O, or S;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen, alkyl optionally substituted with one or more substituents selected from the group consisting of halogen, hydroxy and alkoxy; halogen; haloalkyl; or cyano;

each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

r is an integer of 0 or 1, and

[symbol] represents a single or double bond.

Some embodiments disclosed herein include a compound having the structure of Formula (IX):

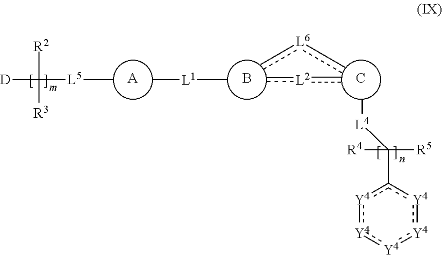

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, halogen, haloalkyl, hydroxy, alkoxy, haloalkoxy, cyano, or oxo;

B is a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, halogen, haloalkyl, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

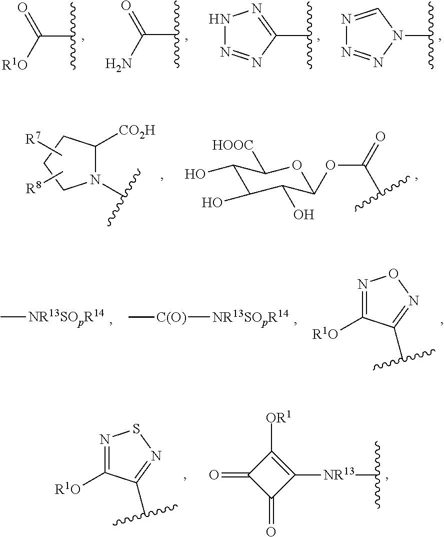

or carboxylic acid isosteres;

L⁴ is

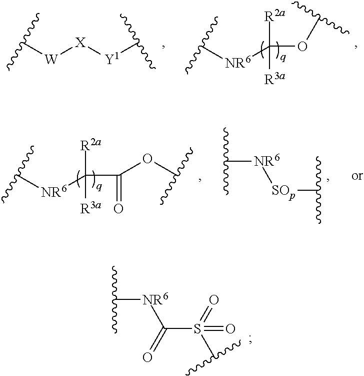

or alternatively,

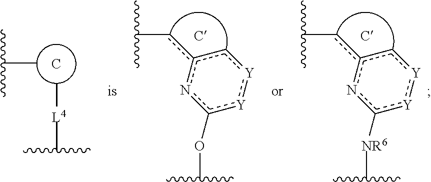

wherein

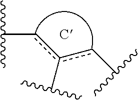

is selected from:

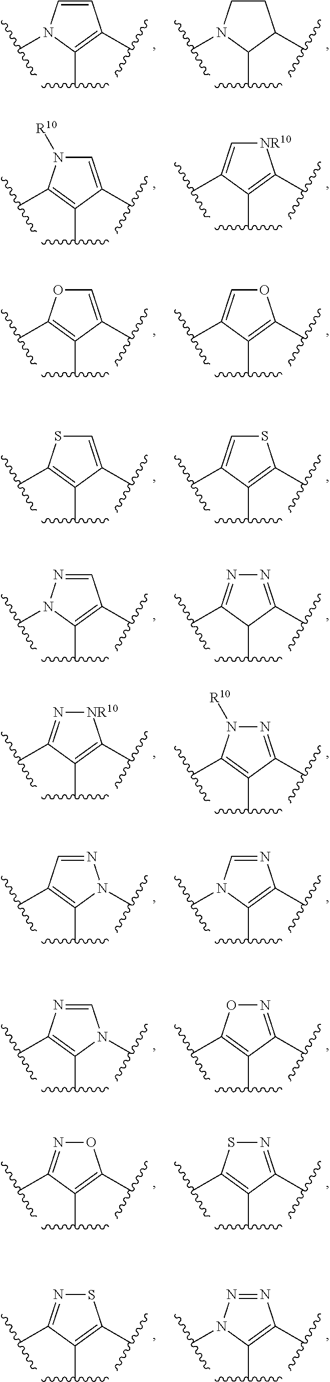

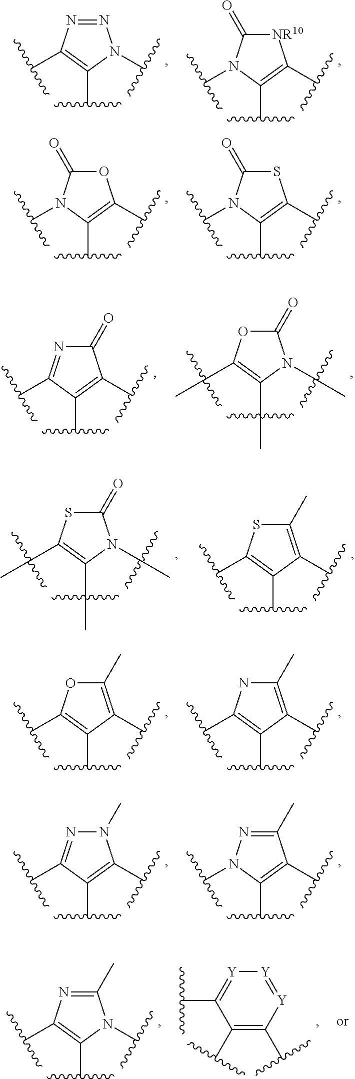

optionally substituted variants thereof;

L¹ is selected from a single bond, a —O— linker, a

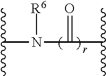

linker, a —C(O)— linker, a —CH₂— linker, a —CH₂O— linker, a —C≡C— linker, a —CH═CH— linker, or a ═C(R¹¹)— linker;

L² is selected from a single bond, a —O— linker, a —NH— linker, a —C(O)— linker, a —CH₂— linker, a —CH₂O— linker, a —C≡C— linker, or a —CH═CH— linker;

L⁵ is selected from a single bond, a —CH₂O— linker, a —CH═CH— linker, a —C≡C— linker, a

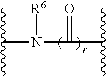

linker, or a 4-7 membered heterocyclyl;

L⁶ is selected from or a

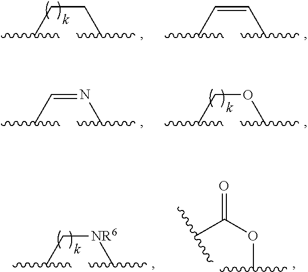

═C(R¹¹)— linker;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen; alkyl optionally substituted with one or more substituents selected from the group consisting of halogen, hydroxy and alkoxy; halogen; haloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl; m is independently an integer from 0-3;

n is an integer from 0-3;

k is an integer from 0-3;

p is an integer from 1-2;

q is an integer from 1-6;

r is an integer of 0 or 1, and

[symbol] represents a single or double bond.

Some embodiments disclosed herein include a compound having the structure of Formula (X):

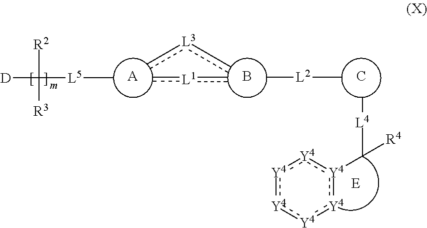

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, halogen, haloalkyl, hydroxy, alkoxy, haloalkoxy, cyano, or oxo;

B is an acetylene or a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, halogen, haloalkyl, hydroxy, alkoxy, haloalkoxy, cyano, or oxo;

C is a ring system selected from the group consisting of 6-11 membered aryl, 5-11 membered heteroaryl, 5-11 membered heterocyclyl, and 5-11 membered carbocyclyl, wherein C is optionally substituted;

E is selected from an optionally substituted 4-11 membered carbocyclyl, an optionally substituted 6-11 membered aryl, an optionally substituted 5-11 membered heteroaryl, or an optionally substituted 4-11 membered heterocyclyl;

D is selected from —OH,

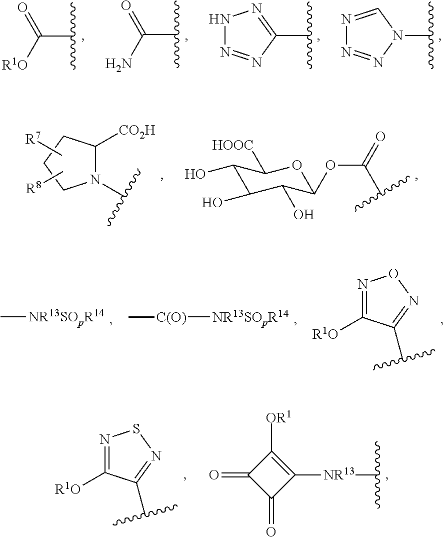

or carboxylic acid isosteres;

L⁴ is

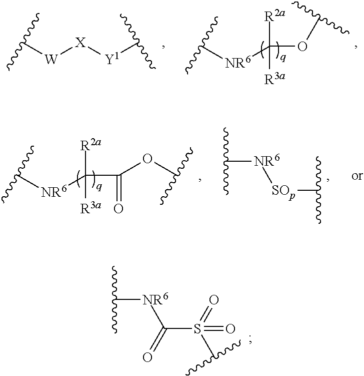

or alternatively,

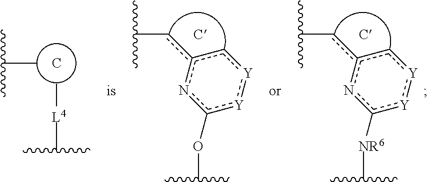

wherein

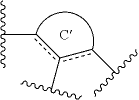

is selected from:

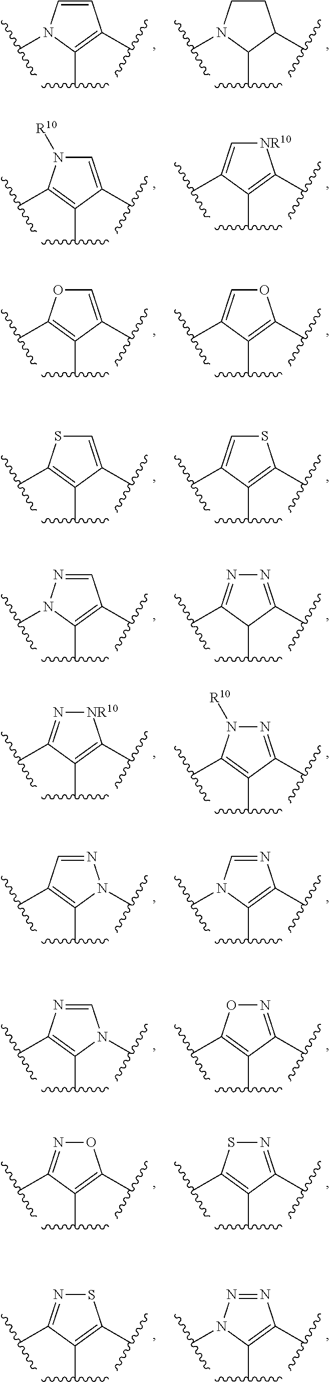

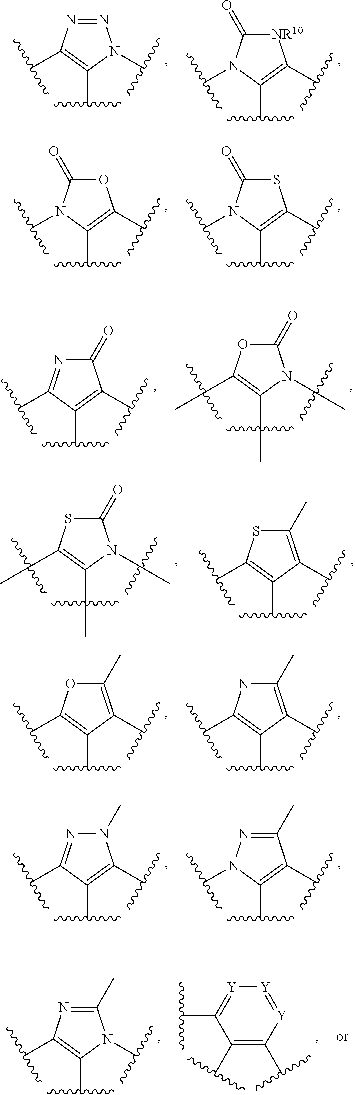

optionally substituted variants thereof;

L¹ is selected from a single bond, a —O— linker, a

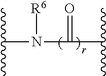

linker, a —C(O)— linker, a —CH₂— linker, a —CH₂O— linker, a —C≡C— linker, a ═C(R¹¹)— linker, or a —CH═CH— linker:

L² is selected from a single bond, a —O— linker, a

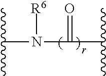

linker, a —C(O)— linker, a —CH₂— linker, a —CH₂O— linker, a —C≡C— linker, or a —CH═CH— linker:

L³ is absent or selected from

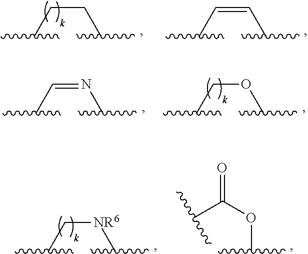

or a ═C(R¹¹)— linker;

L⁵ is selected from a single bond, a —CH₂O— linker, a —CH═CH— linker, a —C≡C— linker, a

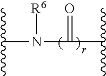

linker or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y⁴ is independently absent, CR⁹, C(R⁹)₂, N, or NH, provided that only one Y⁴ can be absent;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

R⁴ is selected from hydrogen or alkyl optionally substituted with one or more substituents selected from the group consisting of halogen, hydroxy and alkoxy;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or

R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen; alkyl optionally substituted with one or more substituents selected from the group consisting of halogen, hydroxy and alkoxy; halogen; haloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

k is independently an integer from 0-3;

p is an integer from 1-2;

q is an integer from 1-6;

r is an integer of 0 or 1, and

[symbol] represents a single or double bond.

Some embodiments disclosed herein include a compound having the structure of Formula (XI):

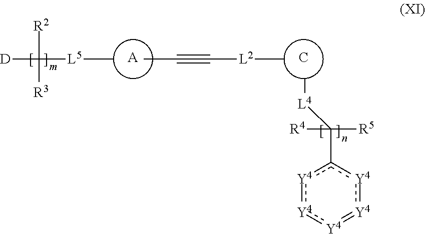

or a pharmaceutically acceptable salt thereof, wherein

A is selected from the group consisting of

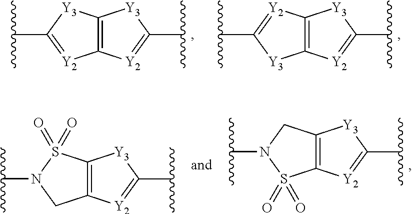

wherein A is unsubstituted or substituted with one or more substituents selected from alkyl, halogen, haloalkyl, hydroxy, alkoxy, haloalkoxy, cyano, or oxo;

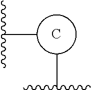

is selected from

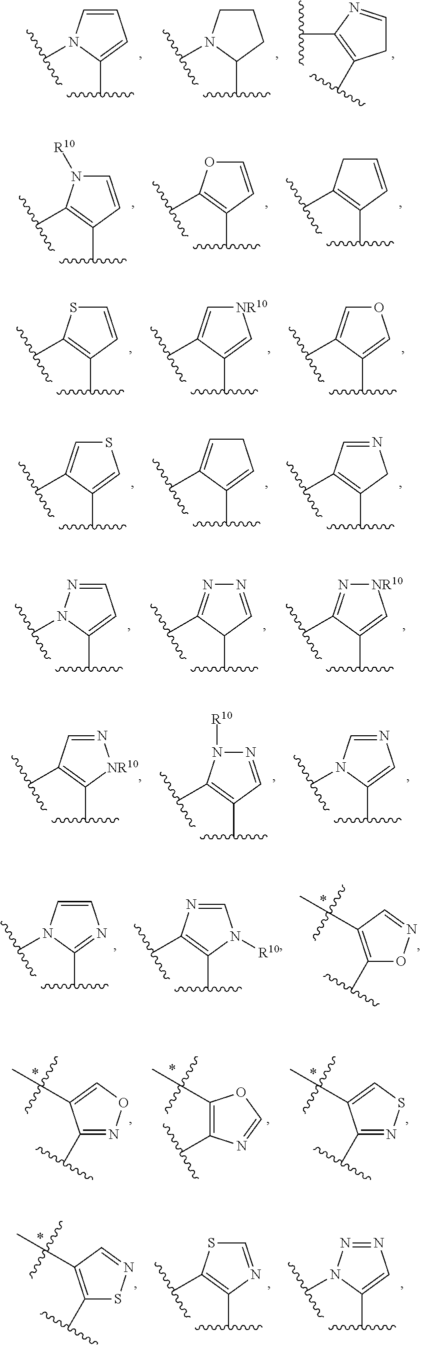

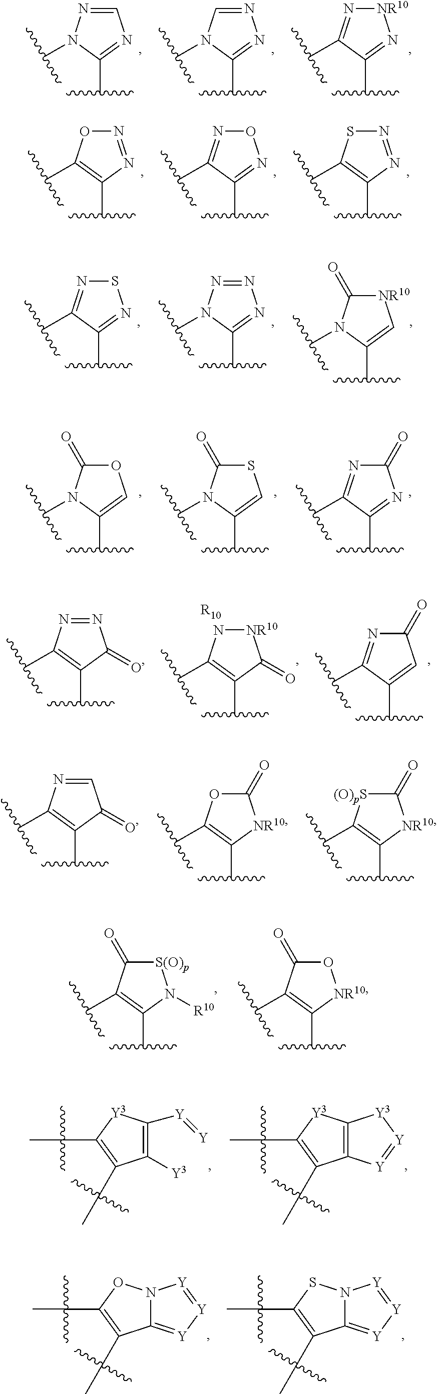

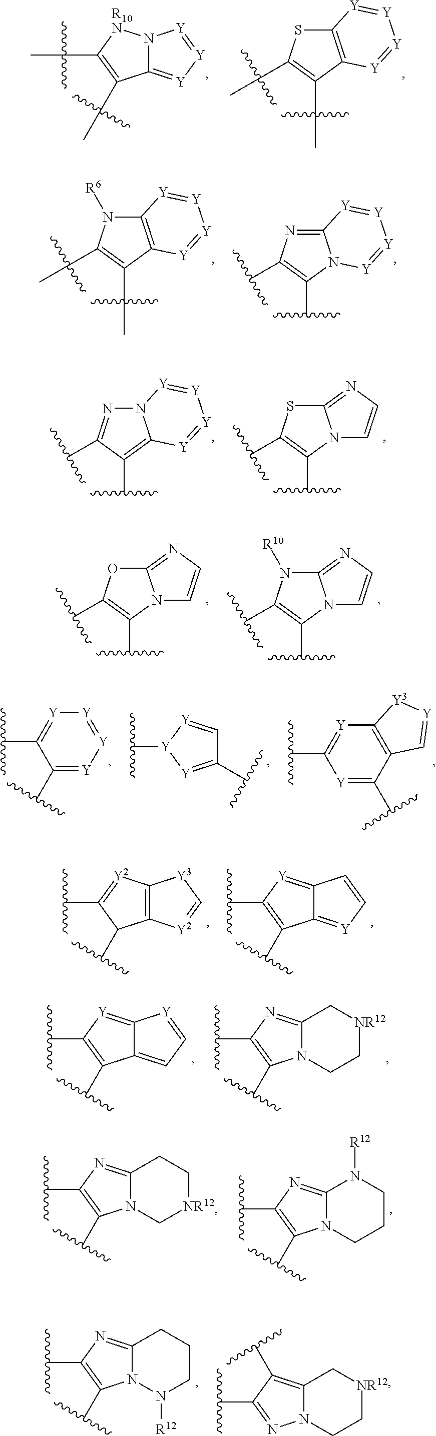

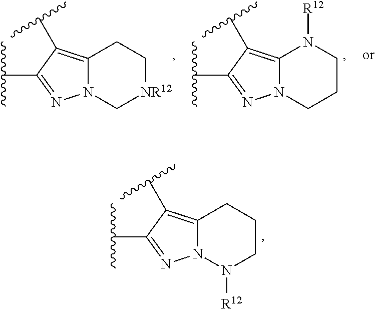

or optionally substituted variants thereof; wherein each * is a point of attachment of C to L²;

D is selected from —OH,

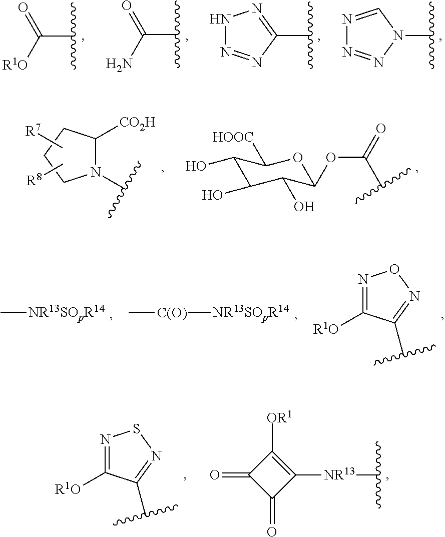

or carboxylic acid isosteres;

L⁴ is

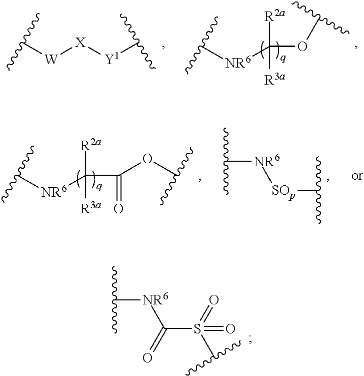

or alternatively,

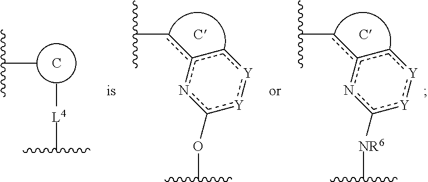

wherein

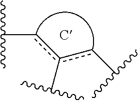

is selected from:

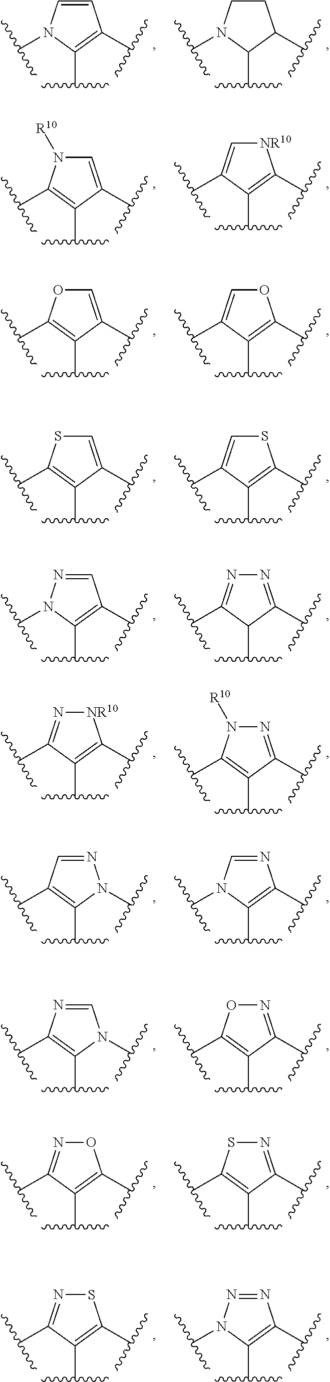

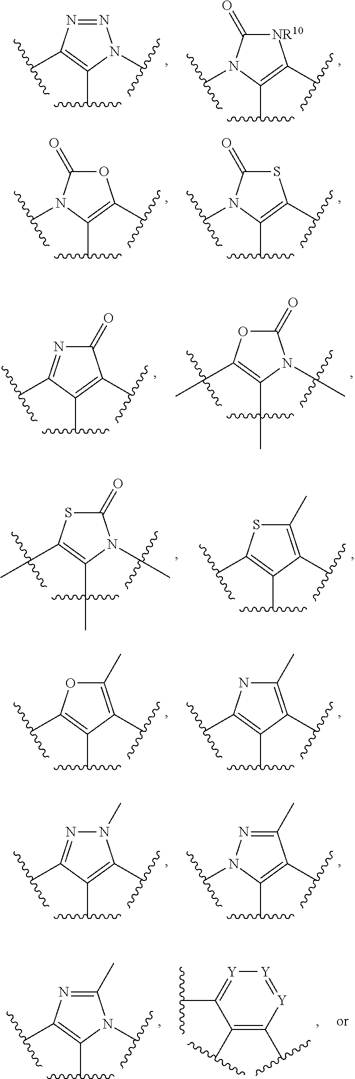

optionally substituted variants thereof;

L² is selected from a single bond, a —O— linker, a

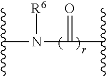

linker, a —C(O)— linker, a —CH₂— linker, a —CH₂O— linker, a —C≡C— linker, or a —CH═CH— linker;

L⁵ is selected from a single bond, a —CH₂O— linker, a —CH═CH— linker, a —C≡C— linker, a

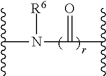

linker, or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

Y² is selected from —CH═ or N;

Y³ is selected from C(R⁶)₂, NR⁶, O or S;

each Y⁴ is independently absent, CR⁹, C(R⁹)₂, N, or NH, provided that only one Y⁴ can be absent;

R¹ is selected from hydrogen or alkyl optionally substituted with one or more substituents selected from the group consisting of halogen, hydroxy, alkoxy, C-amido, O-carboxy, and 5-7 membered heterocyclyl, or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and

R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen, alkyl optionally substituted with one or more substituents selected from the group consisting of halogen, hydroxy and alkoxy, halogen, aryl, or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy, or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen, or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy, halogen, aryl, C₃₋₆ cycloalkyl, or cyano;

each R¹² is independently selected from hydrogen, alkyl optionally substituted with one or more substituents selected from the group consisting of halogen, hydroxy and alkoxy, acyl, C-carboxy, C-amido, sulfinyl, sulfonyl, or S-sulfonamido.

each R¹³ is independently selected from hydrogen, alkyl, haloalkyl, halogen, aryl, or C₃₋₆ cycloalkyl;

each R¹⁴ is independently selected from hydrogen, alkyl, haloalkyl, halogen, aryl, or C₃₋₆ cycloalkyl;

each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

r is an integer of 0 or 1, and

[symbol] represents a single or double bond.

Some embodiments disclosed herein include a pharmaceutical composition comprising an effective amount of a compound of any one of Formulae (I) through (XI), or a pharmaceutically acceptable salt thereof, and a pharmaceutically acceptable carrier, diluent, excipient or combination thereof.

Some embodiments disclosed herein include a method for treating, preventing, reversing, halting, or slowing the progression of a disease or condition selected from fibrosis, cancer, or respiratory disorders, comprising administering an effective amount of a compound of any one of Formulae (I) through (XI), a pharmaceutically acceptable salt thereof, or a pharmaceutical composition thereof to a subject in need thereof. In some embodiments, the disease or condition is fibrosis. In some embodiments, the fibrosis is selected from pulmonary fibrosis, dermal fibrosis, kidney fibrosis, or liver fibrosis. In one embodiment, the fibrosis is idiopathic pulmonary fibrosis. In some embodiments, the respiratory disorders is selected from asthma, COPD, or rhinitis.

Some embodiments disclosed herein include a method of modulating a LPA receptor activity in a cell comprising contacting the cell with an effective amount of a compound of any one of Formulae (I) through (XI), a pharmaceutically acceptable salt thereof, or a pharmaceutical composition thereof to a subject in need thereof. In one embodiment, the LPA receptor is LPA₁.

### DETAILED DESCRIPTION OF THE PREFERRED EMBODIMENT

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. The use of “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The section headings used herein are for organizational purposes only and are not to be construed as limiting the subject matter described.

As used herein, common organic abbreviations are defined as follows:
Ac Acetyl
Ac₂O Acetic anhydride
aq. Aqueous
Bn Benzyl
Bz Benzoyl
BOC or Boc tert-Butoxycarbonyl
Bu n-Butyl
cat. Catalytic
Cbz Carbobenzyloxy
CDI 1,1′-carbonyldiimidazole
° C. Temperature in degrees Centigrade
DBU 1,8-Diazabicyclo[5.4.0]undec-7-ene
DCE 1,2-Dichloroethane
DCM Methylene chloride
DIEA Diisopropylethylamine
DMA Dimethylacetamide
DME Dimethoxyethane
DMF N,N′-Dimethylformamide
DMSO Dimethylsulfoxide
DPPA Diphenylphosphoryl azide
ee % Enantiomeric excess
EA Ethyl acetate
Et Ethyl
EtOAc or EA Ethyl acetate
g Gram(s)
h or hr Hour(s)
HATU 2-(1H-7-azabenzotriazol-1-yl)-1,1,3,3-tetramethyl uronium hexafluorophosphate
HOBT N-Hydroxybenzotriazole
iPr Isopropyl
LCMS Liquid chromatography-mass spectrometry
LDA Lithium diisopropylamide
LiHMDS Lithium bis(trimethylsilyl)amide
m or min Minute(s)
mCPBA meta-Chloroperoxybenzoic Acid
Me Methyl
MeOH Methanol
MeCN Acetonitrile
mL Milliliter(s)
MsCl Methanesulfonyl chloride
MTBE Methyl tertiary-butyl ether
NH₄OAc Ammonium acetate
NIS N-Iodosuccinimide
PE Petroleum ether
PG Protecting group
Pd/C Palladium on activated carbon
Pd(dppf)Cl₂ 1,1′-Bis(diphenylphosphino)ferrocene-palladium(II)dichloride
Ph Phenyl
ppt Precipitate
PMBC 4-Methoxybenzyl chloride
RCM Ring closing metathesis
rt Room temperature
sBuLi sec-Butylithium
SFC Supercritical fluid chromatography
TBAF Tetrabutylammonium fluoride
TEA Triethylamine
TCDI 1,1′-Thiocarbonyl diimidazole
Tert, t tertiary
TFA Trifluoroacetic acid
TFAA Trifluoroacetic acid anhydride
THF Tetrahydrofuran
TLC Thin-layer chromatography
TMEDA Tetramethylethylenediamine
TMSNCO trimethylsilyl isocyanate
μL Microliter(s)

The terms “individual,” “host,” “subject,” and “patient” are broad terms, and are to be given their ordinary and customary meaning to a person of ordinary skill in the art (and are not to be limited to a special or customized meaning), and refer without limitation to a mammal, including, but not limited to, primates, including simians (chimpanzees, apes, monkeys) and humans, cattle, horses, sheep, goats, swine, rabbits, dogs, cats, rodents, rats, mice guinea pigs, or the like.

The term “modulate” as used herein is a broad term, and is to be given its ordinary and customary meaning to a person of ordinary skill in the art (and is not to be limited to a special or customized meaning), and refers without limitation to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein is a broad term, and is to be given its ordinary and customary meaning to a person of ordinary skill in the art (and is not to be limited to a special or customized meaning), and refers without limitation to a molecule that interacts with a target either directly or indirectly. The interactions include, but are not limited to, the interactions of an agonist, partial agonist, an inverse agonist, and antagonist. In one embodiment, a modulator is an antagonist.

The term “agonist” as used herein is a broad term, and is to be given its ordinary and customary meaning to a person of ordinary skill in the art (and is not to be limited to a special or customized meaning), and refers without limitation to a molecule such as a compound, a drug, an enzyme activator or a hormone modulator that binds to a specific receptor and triggers a response in the cell. An agonist mimics the action of an endogenous ligand (such as LPA, prostaglandin, hormone, or neurotransmitter) that binds to the same receptor.

The term “antagonist” as used herein is a broad term, and is to be given its ordinary and customary meaning to a person of ordinary skill in the art (and is not to be limited to a special or customized meaning), and refers without limitation to a molecule such as a compound, which diminishes, inhibits, or prevents the action of another molecule or the activity of a receptor site. Antagonists include, but are not limited to, competitive antagonists, non-competitive antagonists, uncompetitive antagonists, partial agonists, and inverse agonists.

The term “LPA-dependent” as used herein is a broad term, and is to be given its ordinary and customary meaning to a person of ordinary skill in the art (and is not to be limited to a special or customized meaning), and refers without limitation to conditions or disorders that would not occur, or would not occur to the same extent, in the absence of LPA.

The term “LPA-mediated” as used herein is a broad term, and is to be given its ordinary and customary meaning to a person of ordinary skill in the art (and is not to be limited to a special or customized meaning), and refers without limitation to conditions or disorders that might occur in the absence of LPA but can occur in the presence of LPA.

The term “selectivity,” as applied to one LPA receptor versus other LPA receptors, as used herein is a broad term, and is to be given its ordinary and customary meaning to a person of ordinary skill in the art (and is not to be limited to a special or customized meaning), and refers without limitation to a compound that has an IC₅₀ (Ca Flux assay) for the indicated LPA receptor that is at least 10-fold less than the IC₅₀ for other LPA receptors. In some embodiments, selectivity for one LPA receptor versus other LPA receptor means that the compound has an IC₅₀ for the indicated LPA receptor that is at least 10-fold, at least 20-fold, at least 40-fold, at least 50-fold, at least 100-fold, at least 200-fold, at least 500-fold, or at least 1000-fold, less than the IC₅₀ for other LPA receptors. For example, a selective LPA₁ receptor antagonist has an IC₅₀ that is at least 10-fold, at least 20-fold, at least 40-fold, at least 50-fold, at least 100-fold, at least 200-fold, at least 500-fold, or at least 1000-fold, less than the IC₅₀ for other LPA receptors (e.g., LPA2, LPA₃).

The term “pharmaceutical combination” as used herein is a broad term, and is to be given its ordinary and customary meaning to a person of ordinary skill in the art (and is not to be limited to a special or customized meaning), and refers without limitation to a product that results from the mixing or combining of more than one active ingredient and includes both fixed and non-fixed combinations of the active ingredients. The term “fixed combination” means that the active ingredients, e.g., a compound of a preferred embodiment and a co-agent, are both administered to a patient simultaneously in the form of a single entity or dosage. The term “non-fixed combination” means that the active ingredients, e.g., a compound of a preferred embodiment and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and Rb are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo (C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

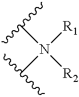

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

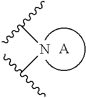

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

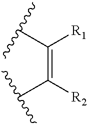

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

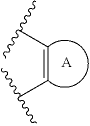

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

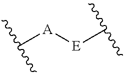

includes the substituent being oriented such that the A is attached at the leftmost attachment point of the molecule as well as the case in which A is attached at the rightmost attachment point of the molecule.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, and —CONRCN, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

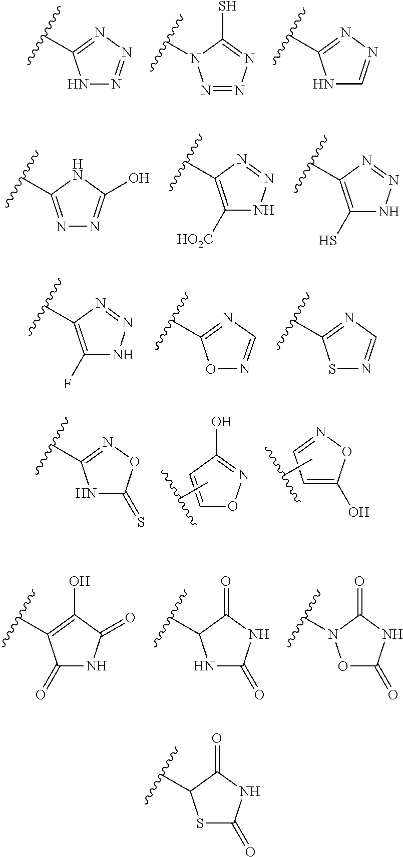

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A therapeutic effect relieves, to some extent, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

The term “pharmaceutically acceptable salt” as used herein is a broad term, and is to be given its ordinary and customary meaning to a person of ordinary skill in the art (and is not to be limited to a special or customized meaning), and refers without limitation to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, and phosphoric acid. Pharmaceutical salts can also be obtained by reacting a compound with an organic acid such as aliphatic or aromatic carboxylic or sulfonic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or naphthalenesulfonic acid. Pharmaceutical salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexylamine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

The term “prodrug” as used herein is a broad term, and is to be given its ordinary and customary meaning to a person of ordinary skill in the art (and is not to be limited to a special or customized meaning), and refers without limitation to a compound or a pharmaceutical composition that can be administered to a patient in a less active or inactive form, which can then be metabolized in vivo into a more active metabolite. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Lysophosphatidic Acid (LPA) Activity

Lysophospholipids (such as lysophosphatidic acid (LPA)) affect fundamental cellular functions that include cellular proliferation, differentiation, survival, migration, adhesion, invasion, and morphogensis. These functions influence many biological processes that include neurogensis, angiogenesis, wound healing, immunity, and carcinogenesis. LPA acts through sets of specific G protein-coupled receptors (GPCRs) in an autocrine and paracrine fashion. LPA binding to its cognate GPCRs (LPA₁, LPA₂, LPA₃, LPA₄, LPA₅, and LPA6) activates intracellular signaling pathways to produce a variety of biological responses. LPA has a role as a biological effector molecule, and has a diverse range of physiological actions such as, but not limited to, effects on blood pressure, platelet activation, and smooth muscle contraction, and a variety of cellular effects, which include cell growth, cell rounding, neurite retraction, and actin stress fiber formation and cell migration. The effects of LPA are predominantly receptor mediated. Activation of the LPA receptors (LPA₁, LPA₂, LPA₃, LPA₄, LPA₅, and LPA6) with LPA mediates a range of downstream signaling cascades. The actual pathway and realized end point are dependent on a range of variables that include receptor usage, cell type, expression level of a receptor or signaling protein, and LPA concentration. Nearly all mammalian cells, tissues, and organs co-express several LPA-receptor subtypes, which indicates that LPA receptors signal in a cooperative manner. LPA₁, LPA₂, and LPA₃ share high amino acid sequence similarity.

A method of treatment of a preferred embodiment comprises inhibiting the physiological activity of LPA in a mammal by administering a therapeutically effective amount of a compound of a preferred embodiment or a pharmaceutically acceptable salt thereof to the mammal in need thereof.

Medicaments for treating a LPA-dependent or LPA-mediated disease or condition in a mammal are provided comprising a therapeutically effective amount of a compound of a preferred embodiment. A compound of a preferred embodiment can also be employed in the manufacture of a medicament for the treatment of a LPA-dependent or LPA-mediated disease or condition. Use of a compound of a preferred embodiment in the treatment or prevention is also provided.

In any of the methods of treatment described herein involving the treatment of LPA dependent diseases or conditions by administration of a compound of a preferred embodiment are also contemplated methods comprising administering at least one additional agent in addition to the compound of preferred embodiments. In various embodiments, each agent is administered in any order, including simultaneously. The compounds of preferred embodiments are useful as antagonists of at least one LPA receptor, or for inhibiting the activity of at least one LPA receptor, or for the treatment of a disease or condition that would benefit from inhibition of the activity of at least one LPA receptor.

The compounds of preferred embodiments, pharmaceutically acceptable salts, pharmaceutically acceptable prodrugs, and pharmaceutically acceptable solvates thereof, which are antagonists of at least one LPA receptor (e.g., LPA₁, LPA₂, LPA₃) can be used to treat patients suffering from one or more LPA-dependent or LPA-mediated conditions or diseases, including, but not limited to, ideopathic pulmonary fibrosis. In some embodiments, LPA-dependent conditions or diseases include those wherein an absolute or relative excess of LPA is present and/or observed.

One or more of the compounds of preferred embodiments can be provided in the form of pharmaceutically acceptable salts, solvates, active metabolites, tautomers, or prodrugs thereof. The compounds of preferred embodiments can be provided in pharmaceutical compositions comprising a therapeutically effective amount of the compound. In some embodiments, the pharmaceutical composition also contains at least one pharmaceutically acceptable inactive ingredient. The pharmaceutical composition can be formulated for intravenous injection, subcutaneous injection, oral administration, inhalation, nasal administration, topical administration, ophthalmic administration, or otic administration. The pharmaceutical composition can be in the form of a tablet, a pill, a capsule, a liquid, an inhalant, a nasal spray solution, a suppository, a suspension, a gel, a colloid, a dispersion, a solution, an emulsion, an ointment, a lotion, an eye drop, or an ear drop.

The pharmaceutical compositions of preferred embodiments can further comprise one or more additional therapeutically active agents other than a compound of the preferred embodiments. Such agents can include, but are not limited to, corticosteroids, immunosuppresants, analgesics, anti-cancer agent, anti-inflammatories, chemokine receptor antagonists, bronchodilators, leukotriene receptor antagonists, leukotriene formation inhibitors, monoacylglycerol kinase inhibitors, phospholipase A₁ inhibitors, phospholipase A₂ inhibitors, and lysophospholipase D (lysoPLD) inhibitors, autotaxin inhibitors, decongestants, antihistamines, mucolytics, anticholinergics, antitussives, expectorants, and β13-2 agonists.

Other objects, features, and advantages of the compounds, methods, and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description

### Compounds

Formula I

Some embodiments disclosed herein include a compound of Formula (I) as described above or a pharmaceutically acceptable salt thereof.

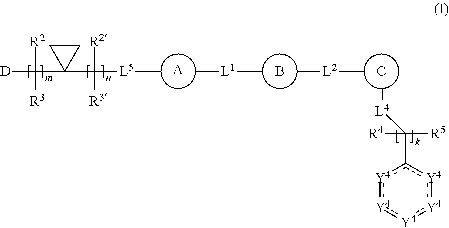

In some embodiments, one of A or B is an acetylene and the other one of A or B is selected from the group consisting of:

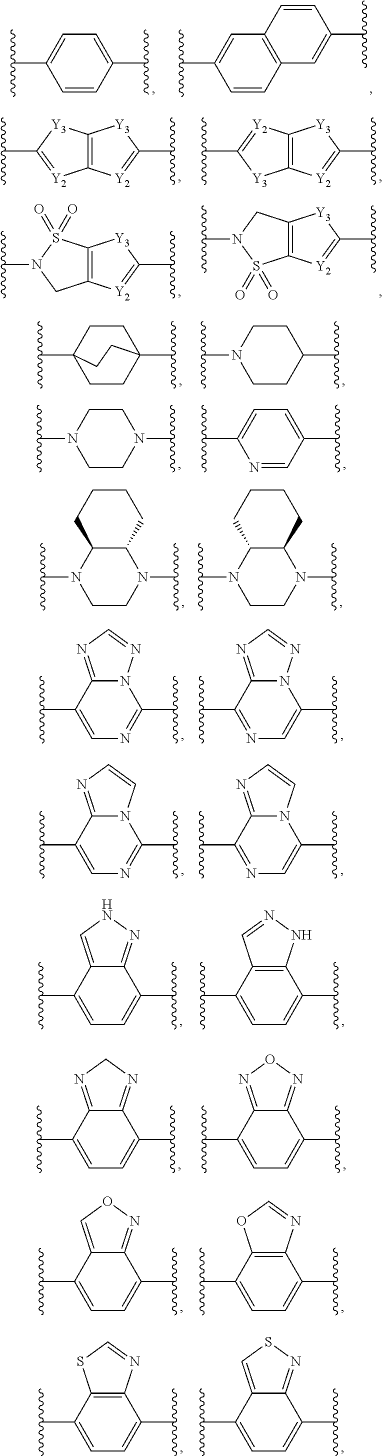

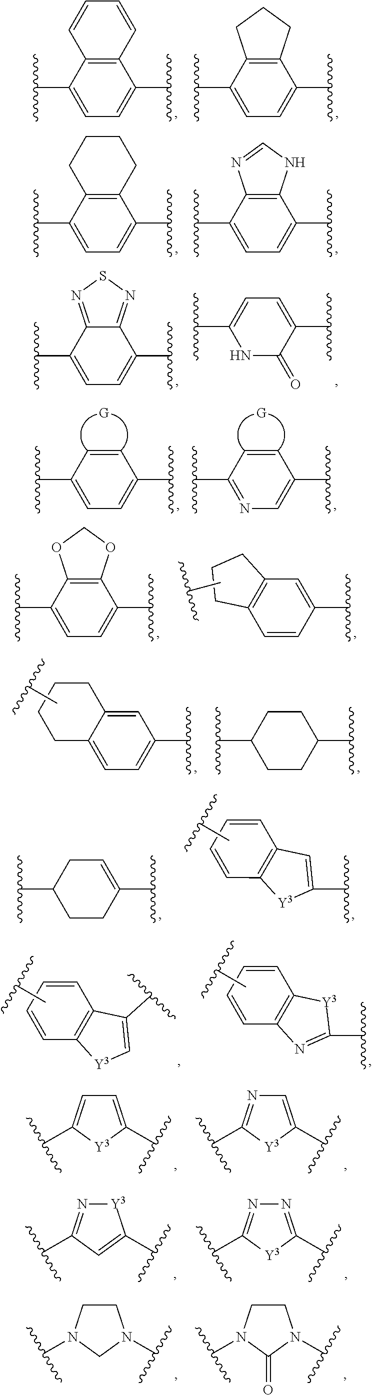

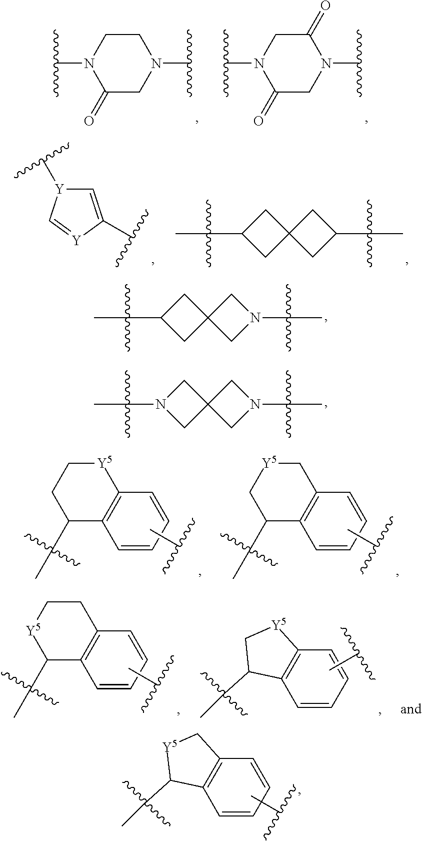

wherein the rings in A or B are unsubstituted or substituted with one or more substituents selected from alkyl, haloalkyl, halogen, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo;

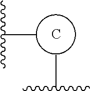

is selected from

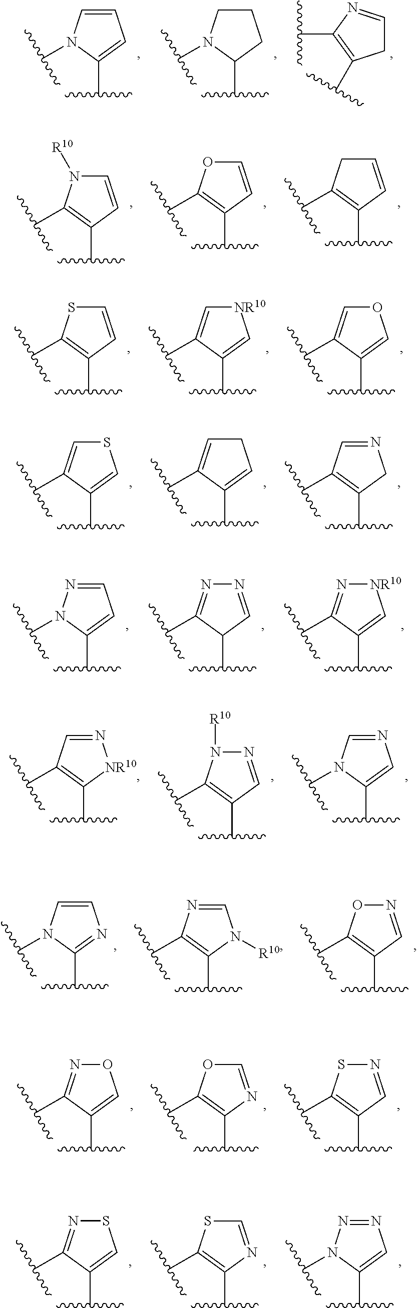

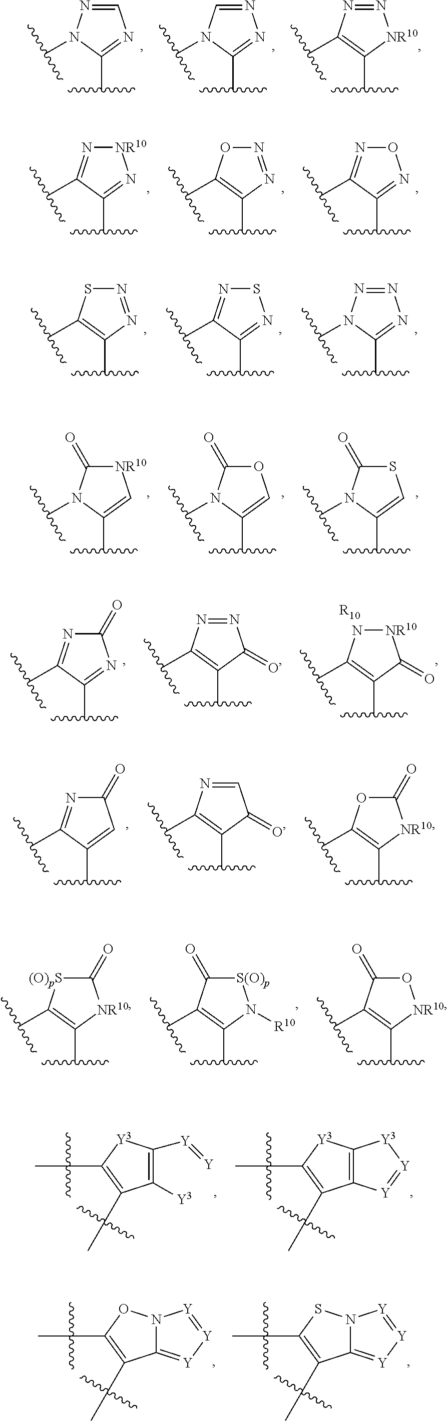

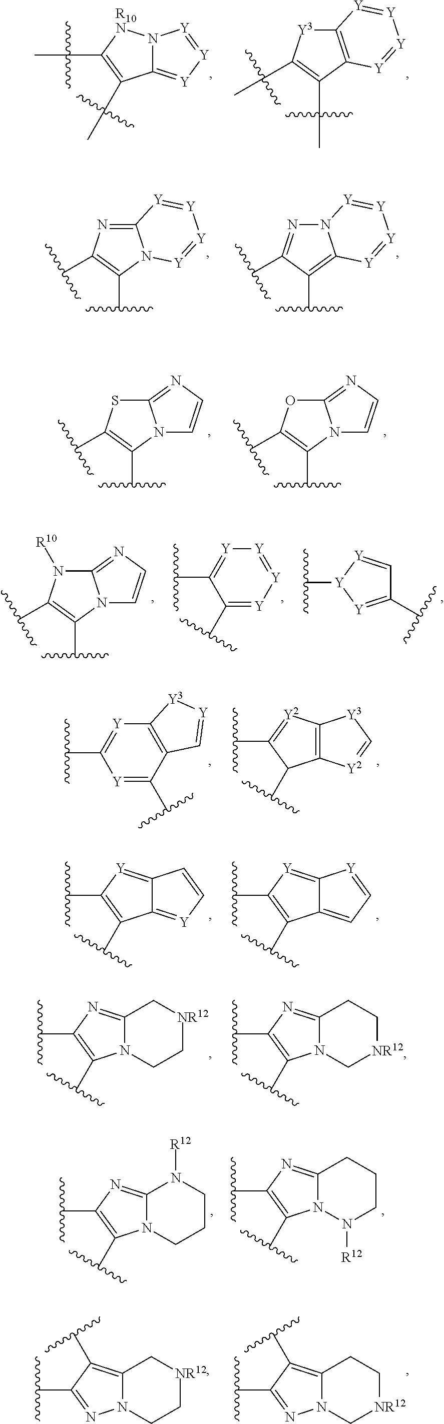

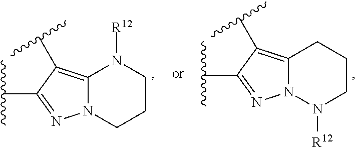

or optionally substituted variants thereof; Y² is selected from —CH═ or N; Y³ is selected from C(R⁶)₂, NR⁶, O or S; Y⁵ is selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (I) is also represented by Formula (Ia):

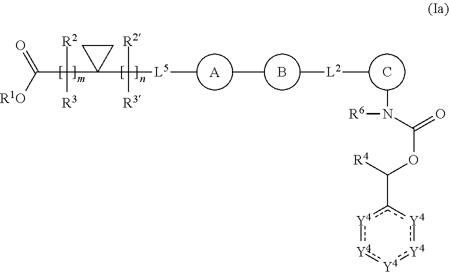

wherein L² and L⁵ are each independently selected from a single bond, a —CH₂O— linker, or a —CH═CH— linker; and R⁴ is selected from hydrogen or alkyl optionally substituted with halogen.

In some embodiments, D is selected from

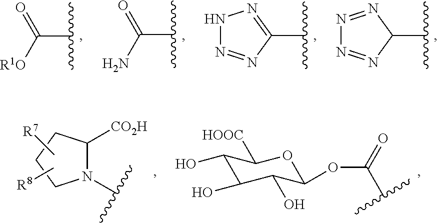

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; and each R¹² is independently selected from hydrogen, alkyl, acyl, C-carboxy, C-amido, sulfinyl, sulfonyl, or S-sulfonamido.

In some embodiments, B is an acetylene and A is selected from

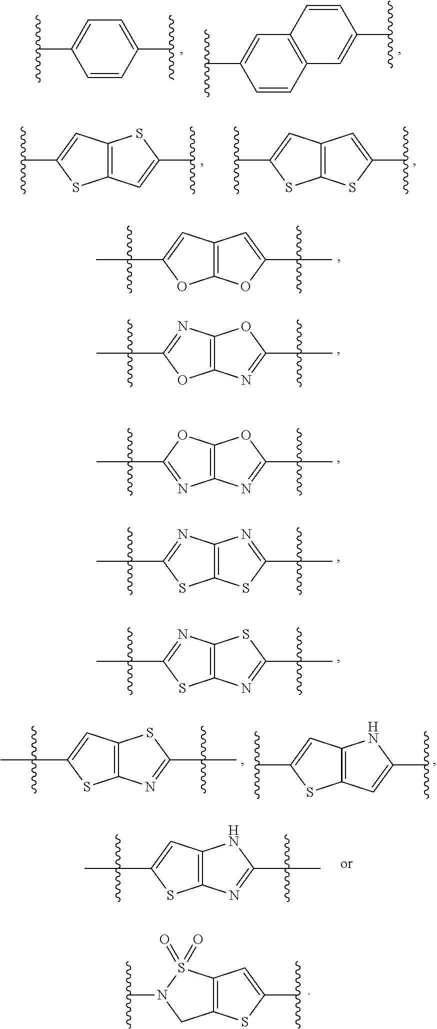

In one embodiment, A is phenyl. In another embodiment, A is naphthyl.

In some embodiments, A is an acetylene and B is selected from

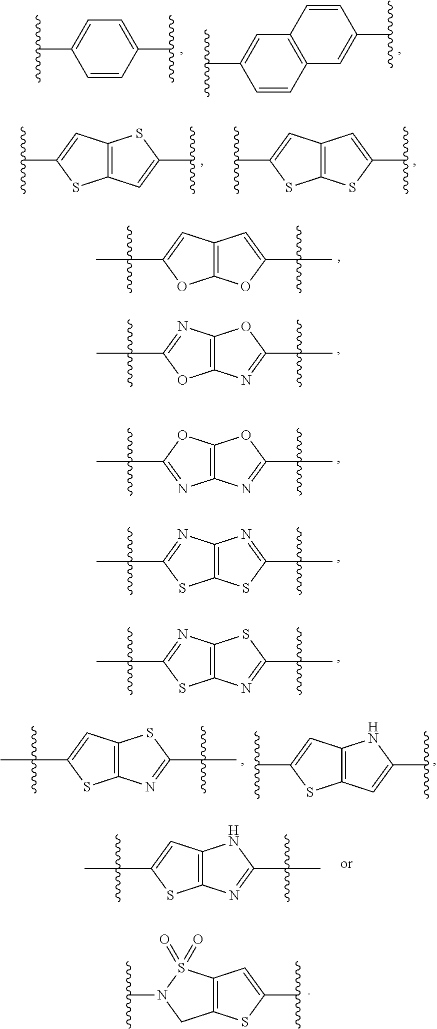

In one embodiment, B is phenyl. In another embodiment, B is naphthyl.

Some embodiments described herein of the compound of Formula (I) or (Ia), rings in A are unsubstituted.

Some embodiments described herein of the compound of Formula (I) or (Ia), rings in B are unsubstituted.

Some embodiments described herein of the compound of Formula (I) or (Ia), rings in A is substituted with one or more substituents selected from alkyl, haloalkyl, halogen, hydroxy, alkoxy, haloalkoxy, cyano, or oxo.

Some embodiments described herein of the compound of Formula (I) or (Ia), rings in B is substituted with one or more substituents selected from alkyl, haloalkyl, halogen, hydroxy, alkoxy, haloalkoxy, cyano, or oxo.

In some embodiments, m is 0 and n is 1. In some other embodiments, m is 1 and n is 0.

In some embodiments, R⁶ is hydrogen. In some embodiments, R¹ is hydrogen.

In some embodiments, each of R², R³, R^{2′} and R^{3′} is hydrogen.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

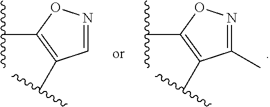

In some embodiments, C is selected from

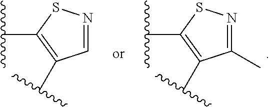

In some embodiments, C is selected from

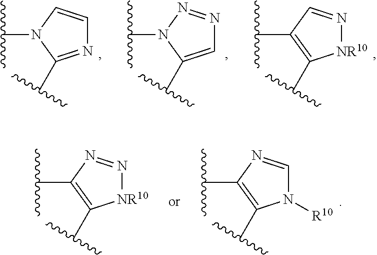

In some such embodiments, R¹⁰ is selected from C₁₋₃ alkyl or C₃₋₆ cycloalkyl. In some such embodiments, R¹⁰ is hydrogen.

In some embodiments, C is selected from

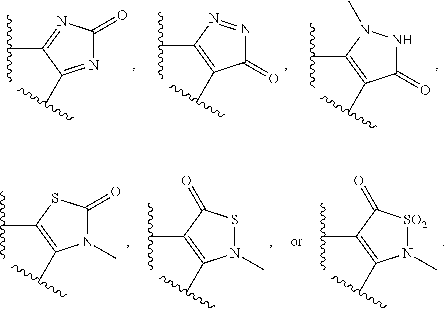

In some embodiments, C is selected from

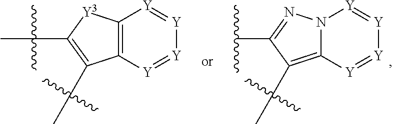

wherein Y³ is selected from O or S. In some such embodiments, each Y is a CR⁶. In some other such embodiments, at least one Y is nitrogen.

In some embodiments, L² is a single bond. In some embodiments, L⁵ is a single bond.

In some embodiments of the compound of Formula (I) or (Ia),

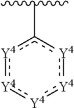

can be

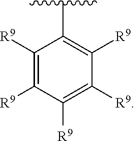

In some such embodiments, each of R⁹ is hydrogen. In some other such embodiments, at least one R⁹ is selected from C₁₋₃ alkyl or halogen.

In some embodiments,

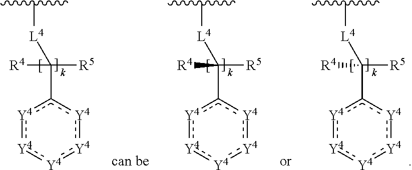

Some embodiments of the compound of Formula (I) are selected from compounds of Table 1 as shown below, and pharmaceutically acceptable salt thereof.

Some embodiments of the compound of Formula (I) are selected from compounds IT001, IT002, IT003 or IT065, as shown in Table 12.

Formula II

Some embodiments disclosed herein include a compound of Formula (II) as described above or a pharmaceutically acceptable salt thereof.

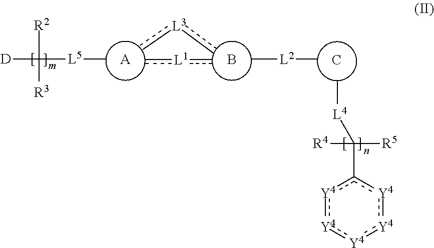

In some embodiments, each of A and B can be an acetylene or selected from the group consisting of:

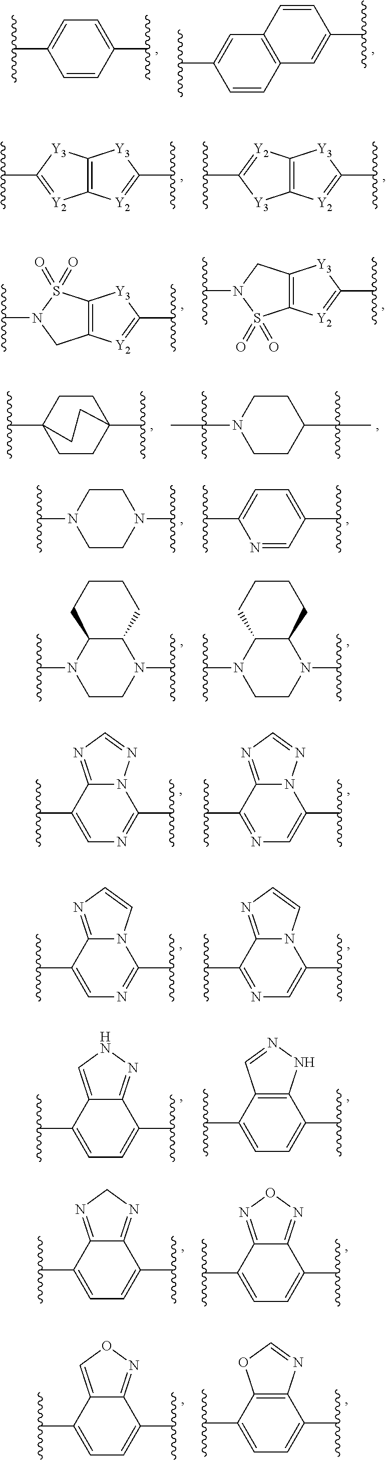

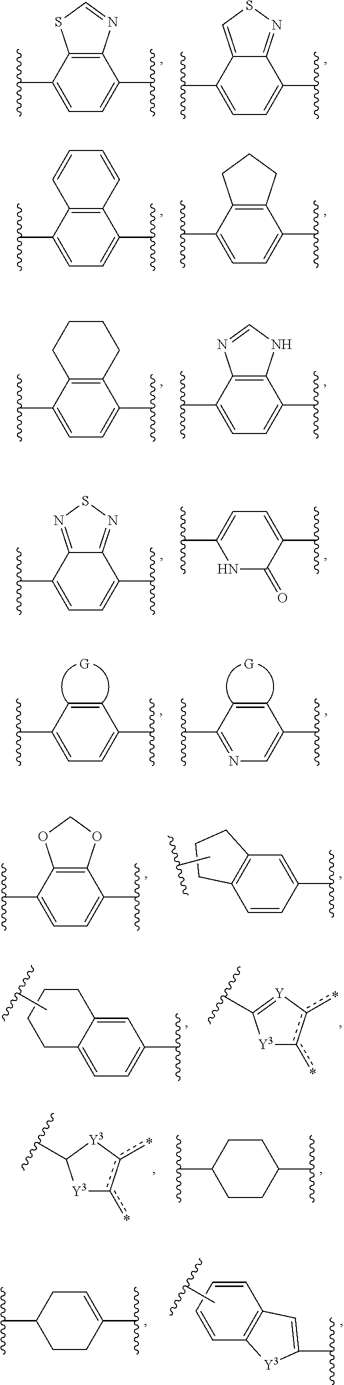

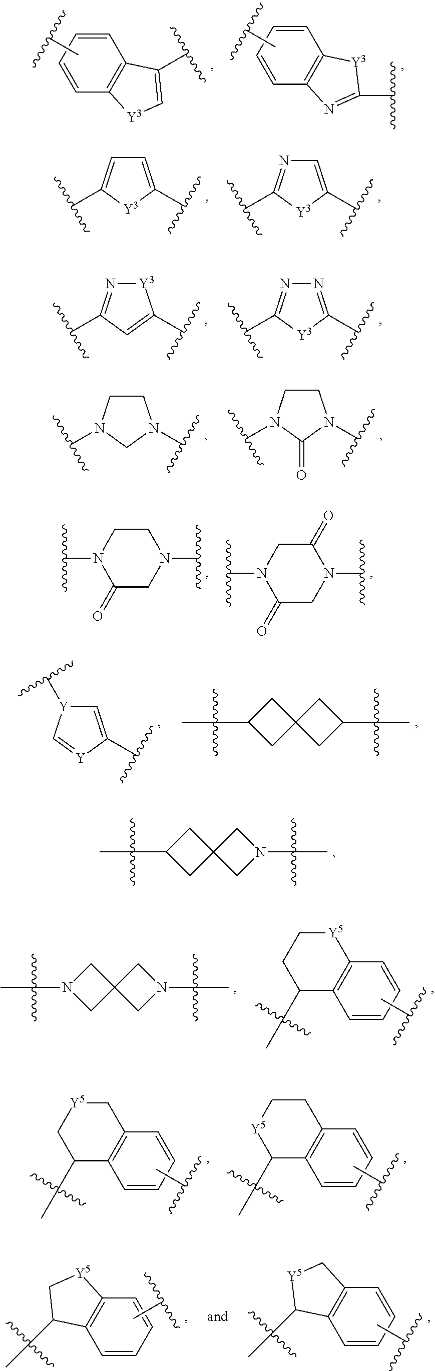

wherein each * is a point of attachment of A or B to L¹ or L³, and wherein the rings in A are unsubstituted or substituted with one or more substituents selected from alkyl, haloalkyl, halogen, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, or oxo;

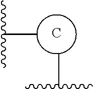

is selected from

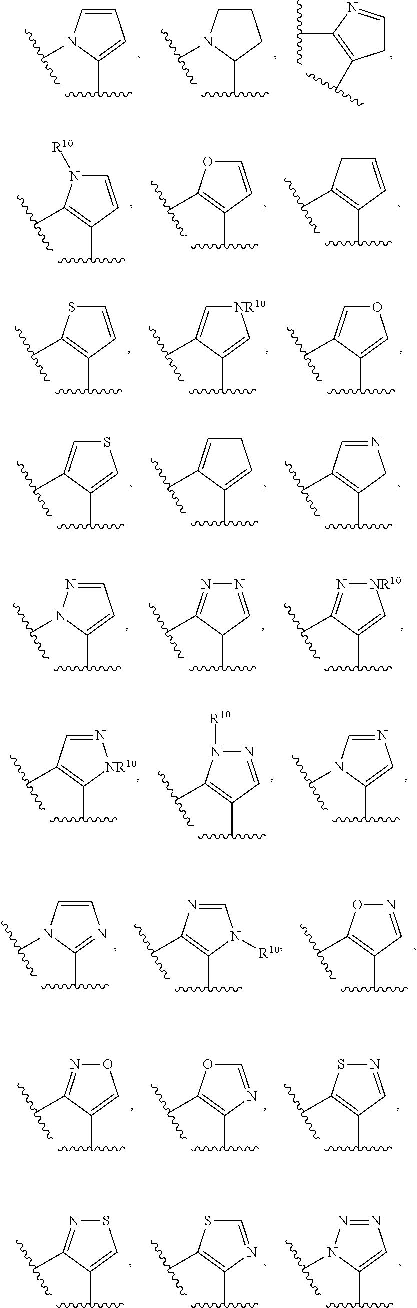

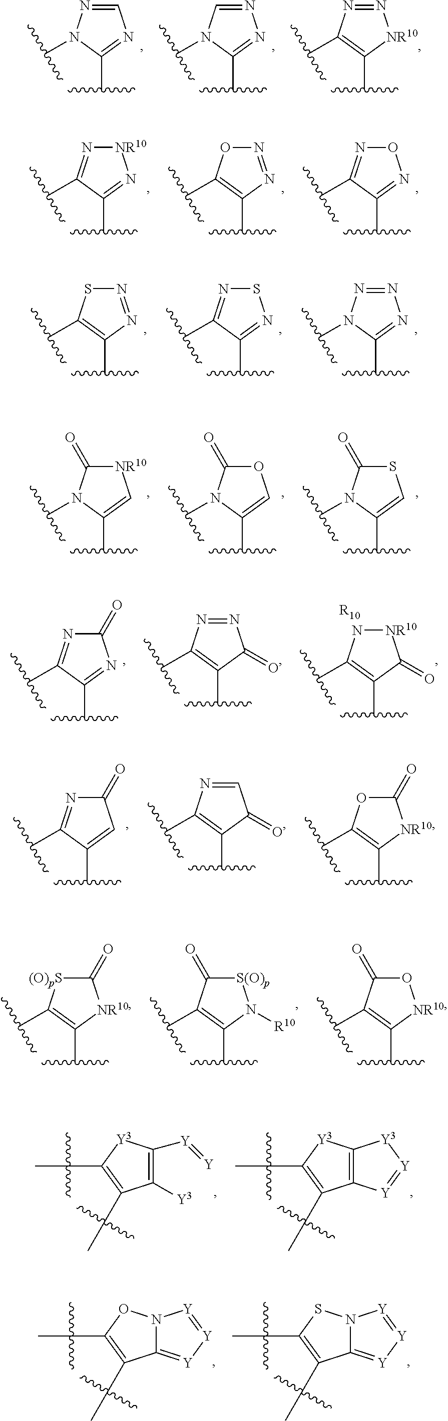

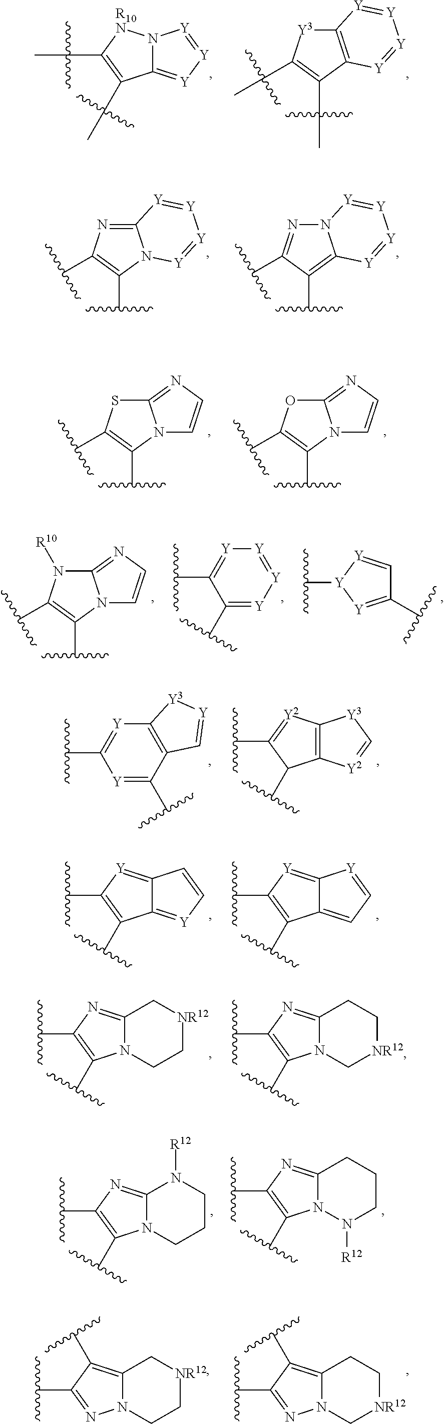

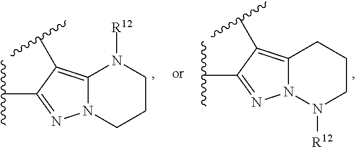

or optionally substituted variants thereof; Y² is selected from —CH═ or N; Y³ is selected from C(R⁶)₂, NR⁶, O or S; Y⁵ is selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (II) is also represented by Formula (IIa):

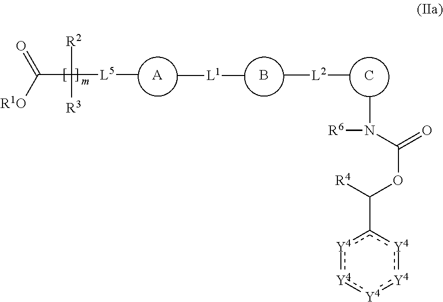

wherein A is selected from acetylene,

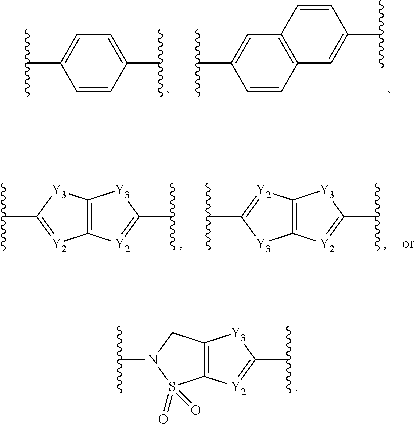

B is selected from acetylene,

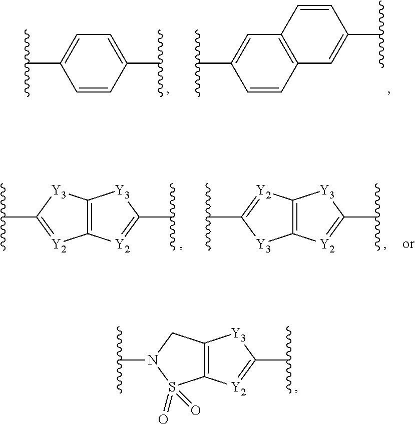

wherein the rings in A or B are unsubstituted or substituted with one or more substituents selected from alkyl, haloalkyl, halogen, hydroxy, alkoxy, haloalkoxy, cyano, or oxo; L¹ is selected from a single bond, a —C(O)— linker, a —CH₂— linker, or a —CH₂O— linker; L² is selected from a single bond, a —O— linker, a —NH— linker, a —C(O)— linker, a —CH₂— linker, or a —CH₂O— linker; and R⁴ is selected from hydrogen or alkyl optionally substituted with halogen.

In some embodiments, D is selected from

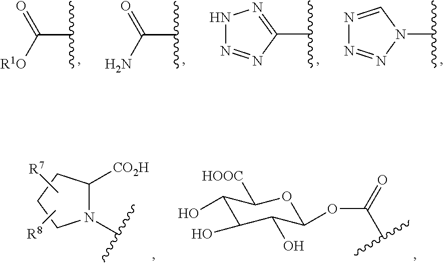

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; and each R¹² is independently selected from hydrogen, alkyl, acyl, C-carboxy, C-amido, sulfinyl, sulfonyl, or S-sulfonamido.

In some embodiments, A is

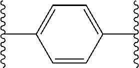 [SMILES: *c1ccc(*)cc1]

and B is selected from acetylene,

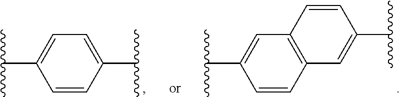 [SMILES: *c1ccc(*)cc1 ; *c1ccc2cc(*)ccc2c1]

In some other embodiments, A is

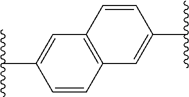 [SMILES: *c1ccc2cc(*)ccc2c1]

and B is selected from acetylene or

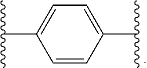 [SMILES: *c1ccc(*)cc1]

In one such embodiment, B is acetylene.

In some embodiments, B is

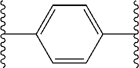 [SMILES: *c1ccc(*)cc1]

and A is selected from acetylene,

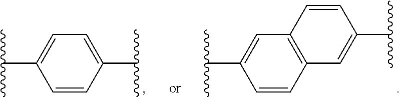 [SMILES: *c1ccc(*)cc1 ; *c1ccc2cc(*)ccc2c1]

In some other embodiments, B is

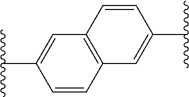 [SMILES: *c1ccc2cc(*)ccc2c1]

and A is selected from acetylene or

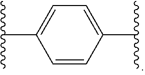 [SMILES: *c1ccc(*)cc1]

In one such embodiment, A is acetylene.

Some embodiments described herein of the compound of Formula (II) or (IIa), rings in A are unsubstituted.

Some embodiments described herein of the compound of Formula (II) or (IIa), rings in B are unsubstituted.

Some embodiments described herein of the compound of Formula (II) or (IIa), rings in A is substituted with one or more substituents selected from alkyl, haloalkyl, halogen, hydroxy, alkoxy, haloalkoxy, cyano, or oxo.

Some embodiments described herein of the compound of Formula (II) or (IIa), rings in B is substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

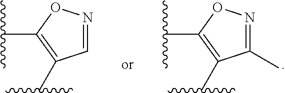

In some embodiments, C is selected from

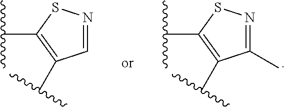

In some embodiments, C is selected from

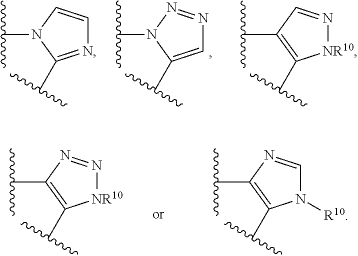

In some such embodiments, R¹⁰ is selected from C₁₋₃ alkyl or C₃₋₆ cycloalkyl. In some such embodiments, R¹⁰ is hydrogen.

In some embodiments, C is selected from

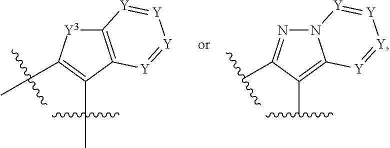

wherein Y³ is selected from O or S. In some such embodiments, Y is a CR⁶. In some other such embodiments, at least one Y is nitrogen.

In some embodiments, C is selected from

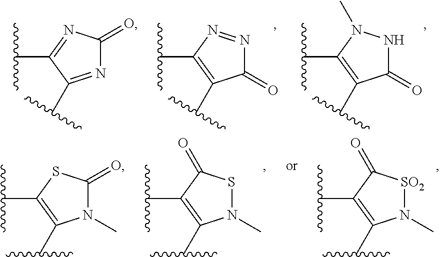

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, R⁶ is hydrogen.

In some embodiments, L¹ is L² is a single bond. In some embodiments, L is a single bond.

In some embodiments, L⁵ is —NH—. In some other embodiments, L⁵ is —C(O)—NH—. In still some other embodiments, L⁵ is —C≡C—.

In some embodiments, R¹ is hydrogen.

Some embodiments of the compound of Formula (II) or (IIa),

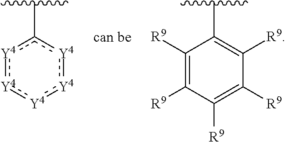

In some such embodiments, each of R⁹ is hydrogen. In some other such embodiments, at least one R⁹ is selected from C₁₋₃ alkyl or halogen.

In some embodiments,

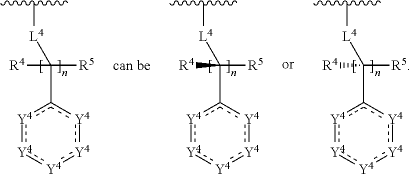

Some embodiments of the compound of Formula (II) are selected from compounds of Table 2 as shown below, and pharmaceutically acceptable salt thereof.

Some embodiments of the compound of Formula (II) are selected from compounds IT005 or IT006, as shown in Table 12.

Formula III

Some embodiments disclosed herein include a compound of Formula (III) as described above or a pharmaceutically acceptable salt thereof.

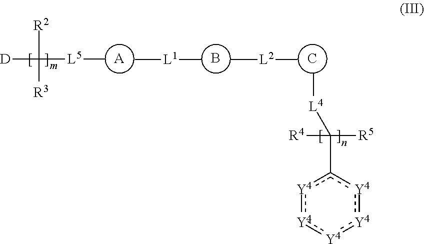

In some embodiments, one of A or B is selected from the group consisting of

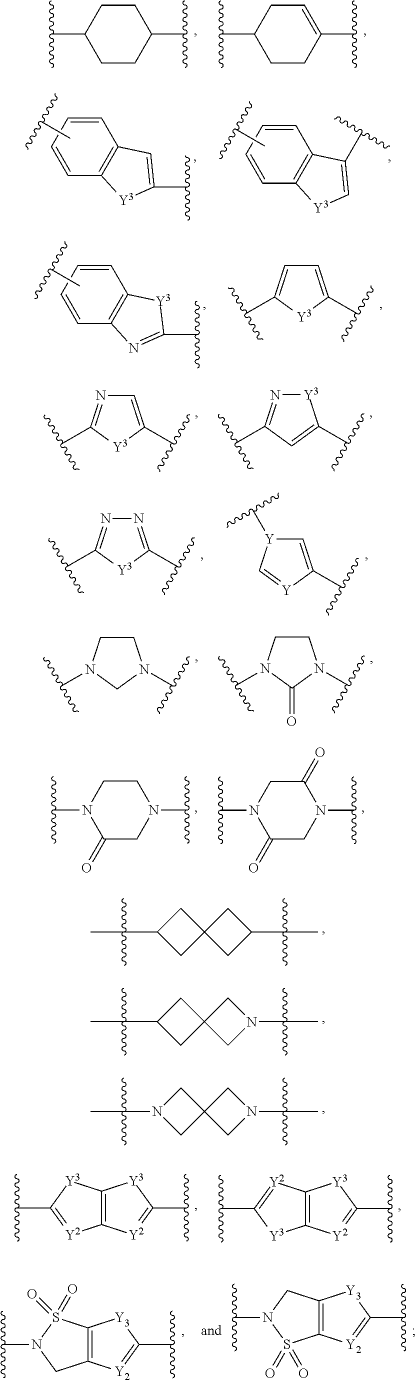

and the other one of A or B is selected from

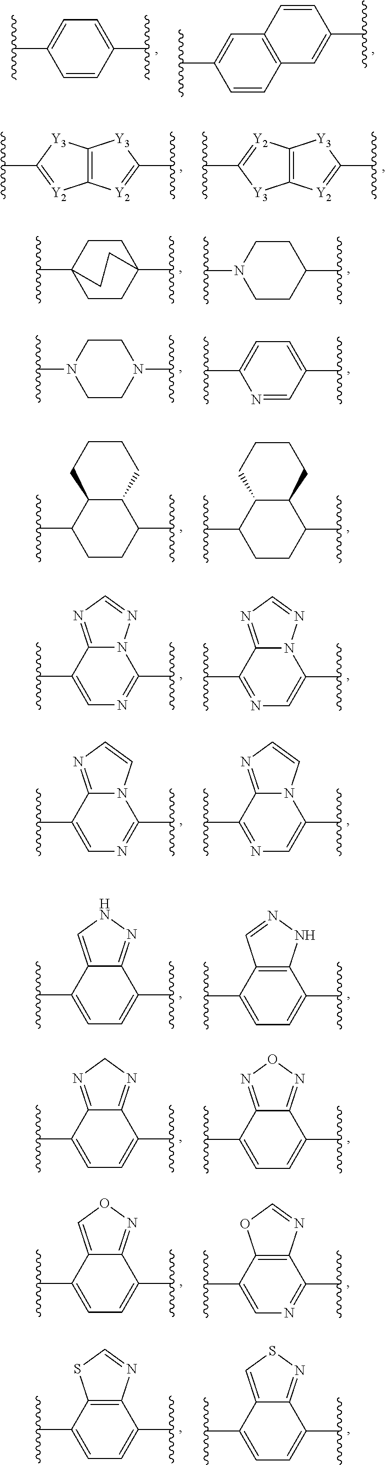

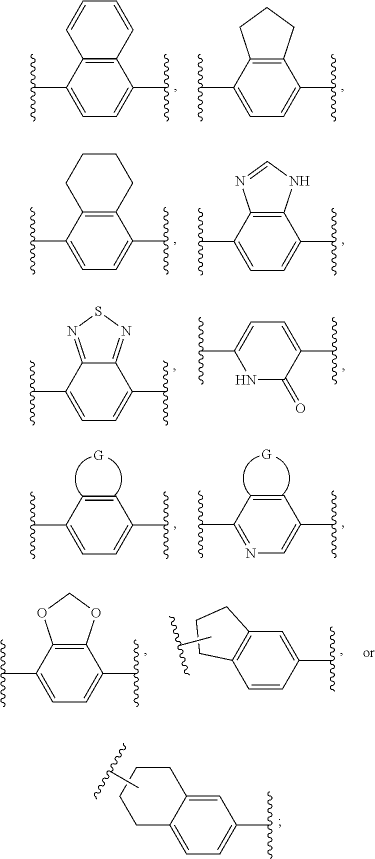

wherein each of A and B is unsubstituted or substituted with one or more substituents selected from alkyl, halogen, haloalkyl, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, or oxo;

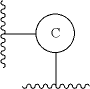

is selected from

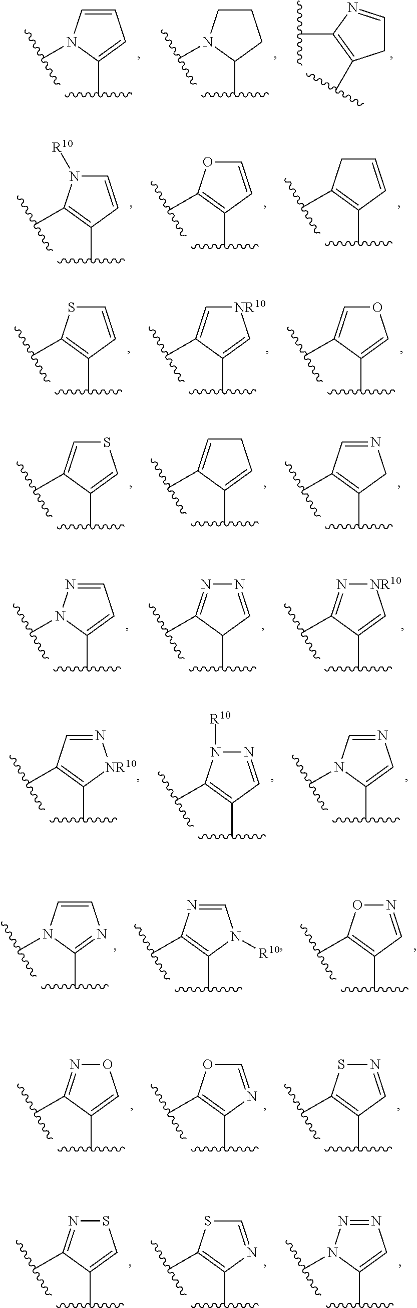

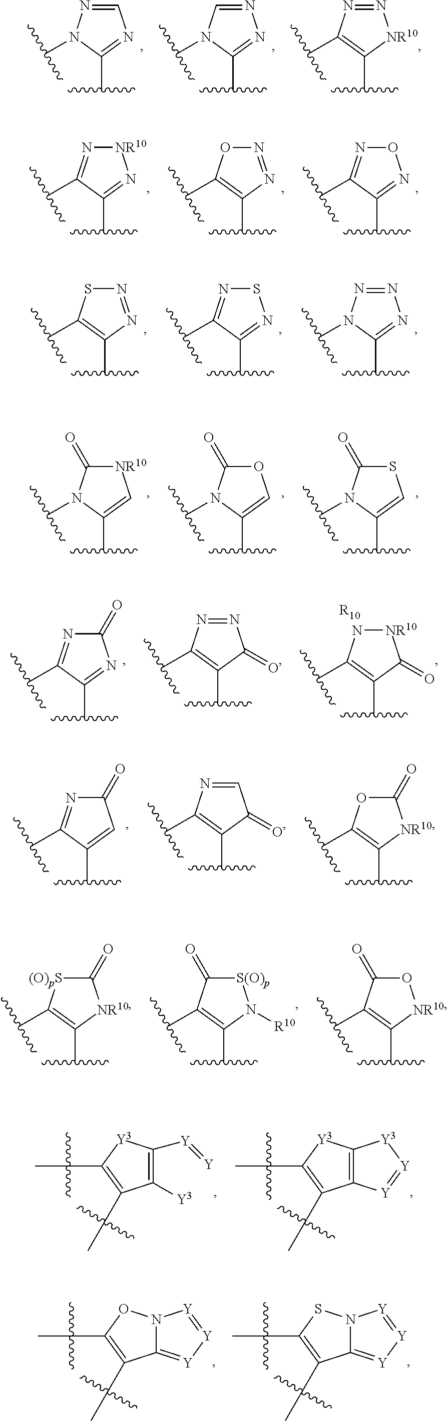

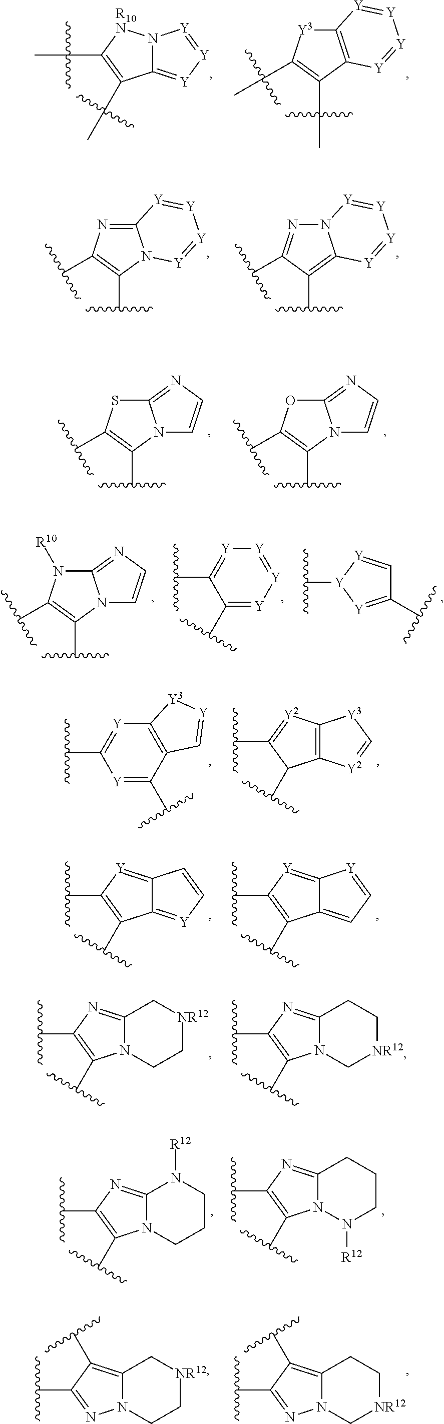

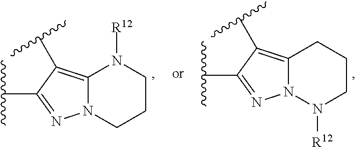

or optionally substituted variants thereof; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, one of A or B is selected from

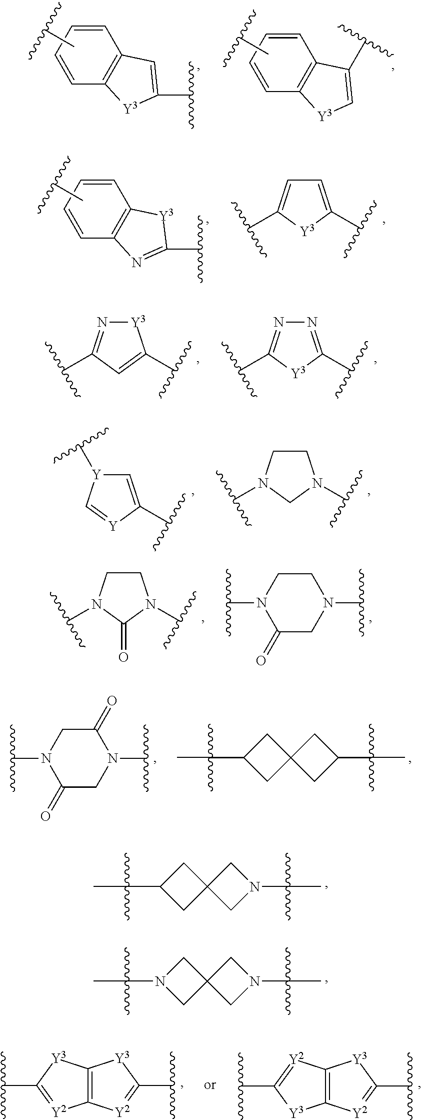

and the other one of A or B is selected from

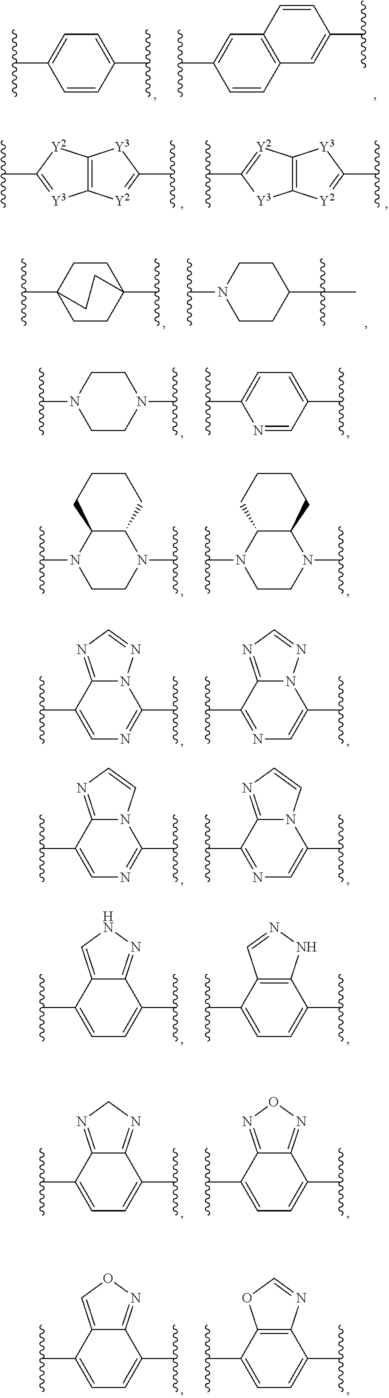

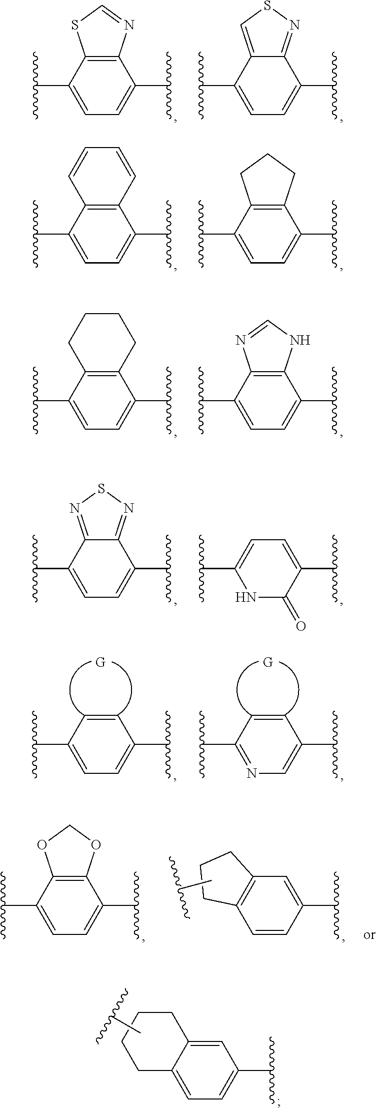

wherein each of A and B is unsubstituted or substituted with one or more substituents selected from alkyl, halogen, haloalkyl, hydroxy, alkoxy, haloalkoxy, cyano, or oxo.

In some embodiments, the compound of Formula (III) is also represented by Formula (IIIa):

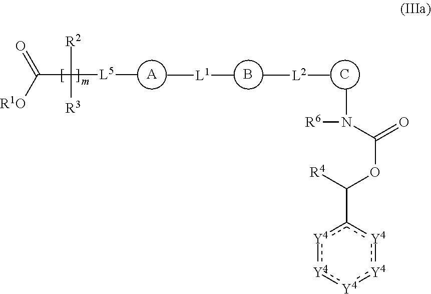

wherein one of A or B is selected from

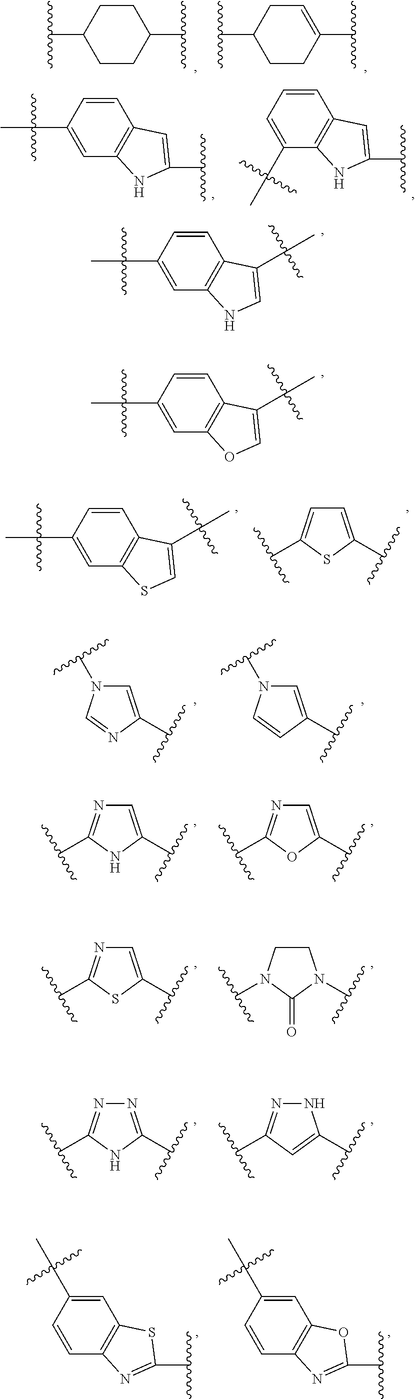

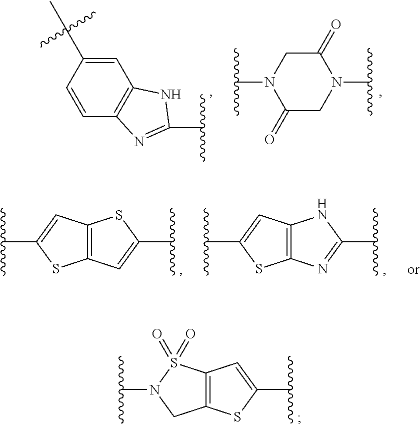

and the other one of A or B is selected from

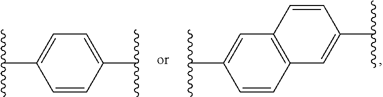 [SMILES: *c1ccc(*)cc1 ; *c1ccc2cc(*)ccc2c1]

wherein rings in A and B can each be unsubstituted or substituted with one or more substituents selected from alkyl, haloalkyl, halogen, hydroxy, alkoxy, haloalkoxy, cyano, or oxo. R⁴ is hydrogen or alkyl optionally substituted with halogen. In some such embodiments, A is a phenyl. In some further embodiments, A is substituted with one or more halogen. In some such embodiments, B is a phenyl. In some other such embodiments, B is a naphthyl. In some further embodiments, B is substituted with one or more halogen.

In some embodiments, one of A or B is selected from

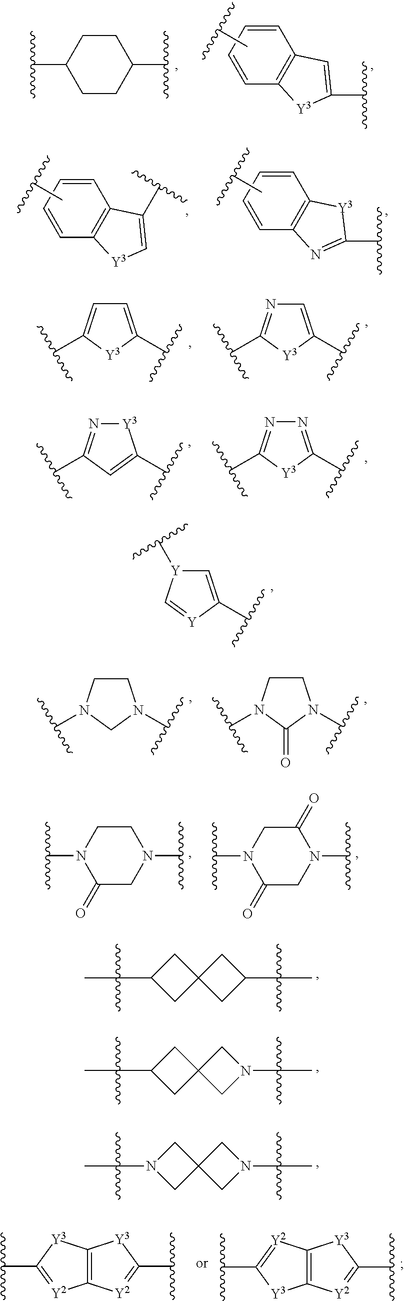

and the other A or B is a ring system selected from the group consisting of 6-11 membered aryl, 5-11 membered heteroaryl, 4-11 membered heterocyclyl, and 4-11 membered carbocyclyl; wherein rings in A or B are unsubstituted or substituted with one or more substituents selected from alkyl, halogen, hydroxy, alkoxy, haloalkoxy, cyano, or oxo; D is selected from

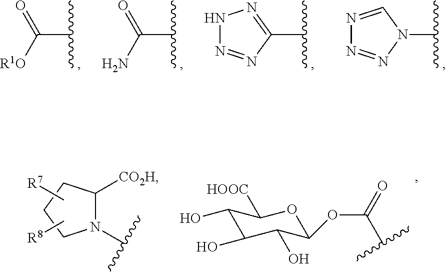

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; and each R¹² is independently selected from hydrogen, alkyl, acyl, C-carboxy, C-amido, sulfinyl, sulfonyl, or S-sulfonamido

Some embodiments described herein of the compound of Formula (III) or (Ma), rings in A are unsubstituted.

Some embodiments described herein of the compound of Formula (III) or (Ma), rings in B are unsubstituted.

Some embodiments described herein of the compound of Formula (III) or (Ma), rings in A is substituted with one or more substituents selected from alkyl, haloalkyl, halogen, hydroxy, alkoxy, haloalkoxy, cyano, or oxo.

Some embodiments described herein of the compound of Formula (III) or (Ma), rings in B is substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

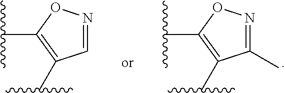

In some embodiments, C is selected from

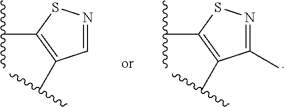

In some embodiments, C is selected from

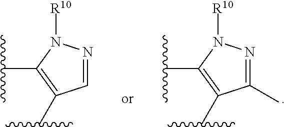

In some such embodiments, R¹⁰ is hydrogen. In some such embodiments. R¹⁰ is C₁₋₃ alkyl or C₃₋₆ cycloalkyl

In some embodiments, C is

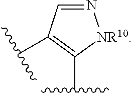

In some embodiments, C is

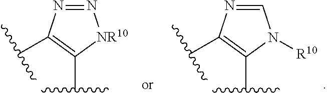

In some such embodiments, R¹⁰ is C₁₋₃ alkyl or C₃₋₆ cycloalkyl. In some such embodiments, R¹⁰ is hydrogen.

In some embodiments, C is selected from

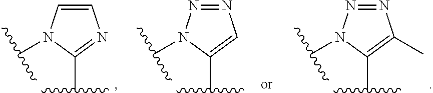

In some embodiments, C is selected from

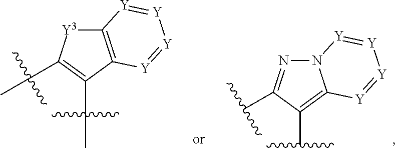

wherein Y³ is selected from O or S. In some such embodiments, Y is a CR⁶. In some other such embodiments, at least one Y is nitrogen.

In some embodiments, C is selected from

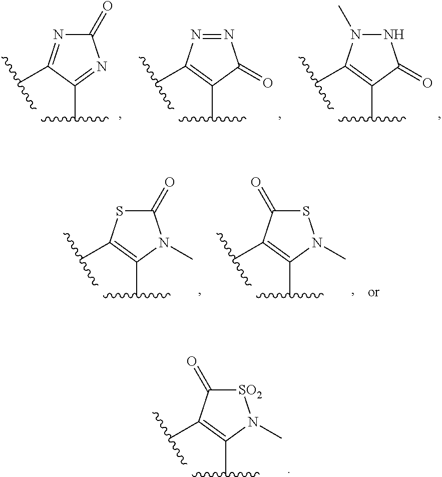

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, L¹ L¹ is a single bond. In some other embodiments, L is —C≡C— linker. In still some other embodiments, L¹ is a

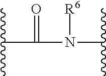

linker. In some of such embodiments, L¹ is a —C(O)—NH— linker.

In some embodiments, R⁶ is hydrogen. In some embodiments, R¹ is hydrogen.

R⁴ is selected from hydrogen or alkyl optionally substituted with halogen. In some other embodiments, R⁴ is unsubstituted alkyl.

Some embodiments of the compound of Formula (III) or (IIIa),

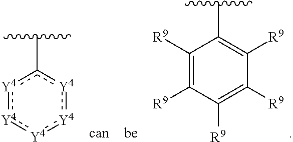

In some such embodiments, each of R⁹ is hydrogen. In some other such embodiments, at least one R⁹ is selected from C₁₋₃ alkyl or halogen.

In some embodiments,

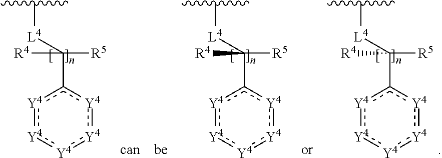

Some embodiments of the compound of Formula (III) are selected from compounds of Table 3 as shown below, and pharmaceutically acceptable salt thereof.

Some embodiments of the compound of Formula (III) are selected from compounds IT007-IT010, IT025, IT046, IT050, IT051, IT053, IT054, IT056, IT059, IT060, IT066, IT067, IT071 or IT091, as shown in Table 12.

Formula IV

Some embodiments disclosed herein include a compound of Formula (IV) as described above or a pharmaceutically acceptable salt thereof.

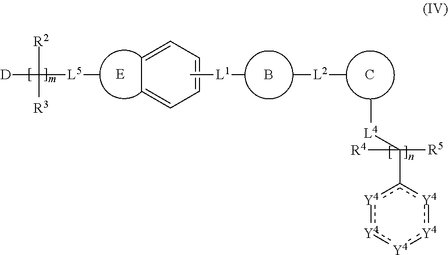

In some embodiments, B is selected from the group consisting of

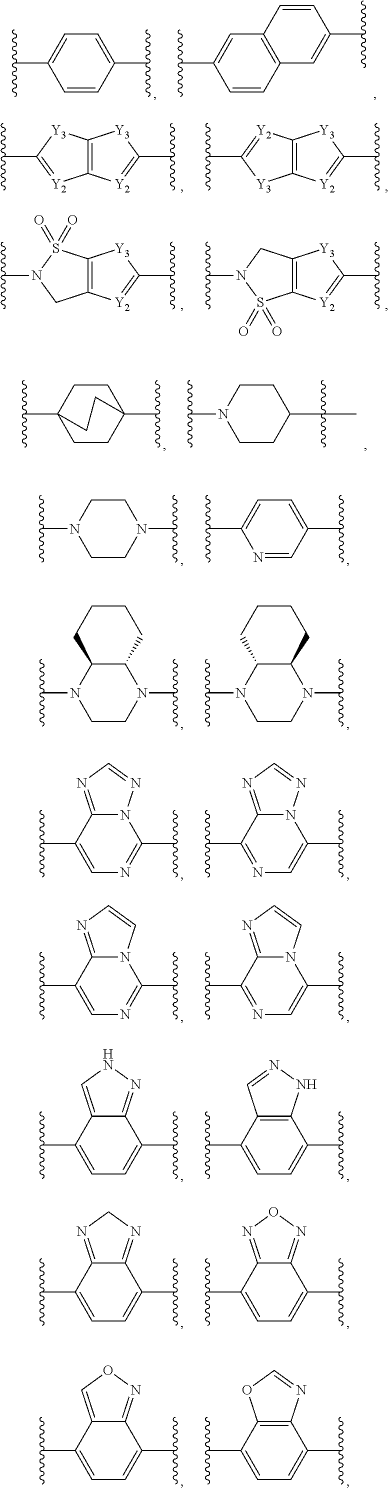

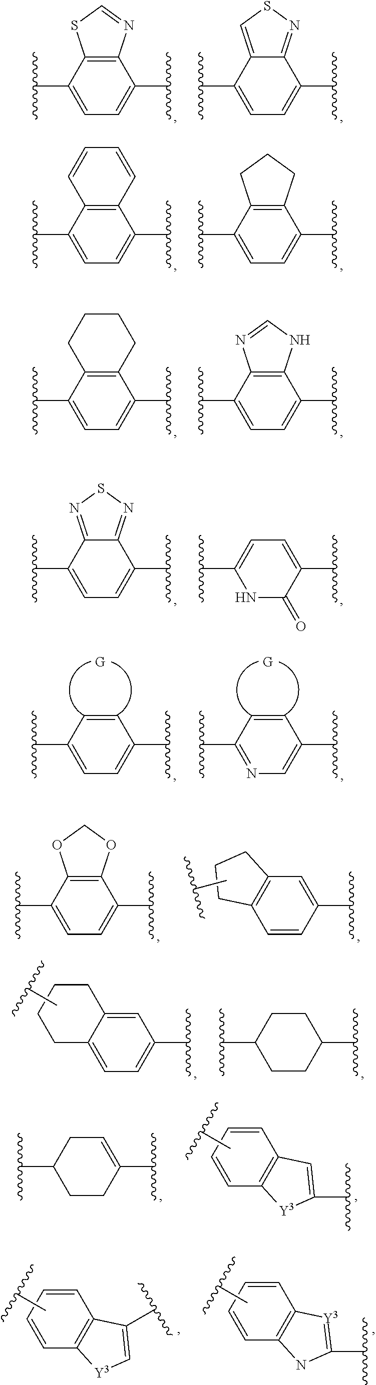

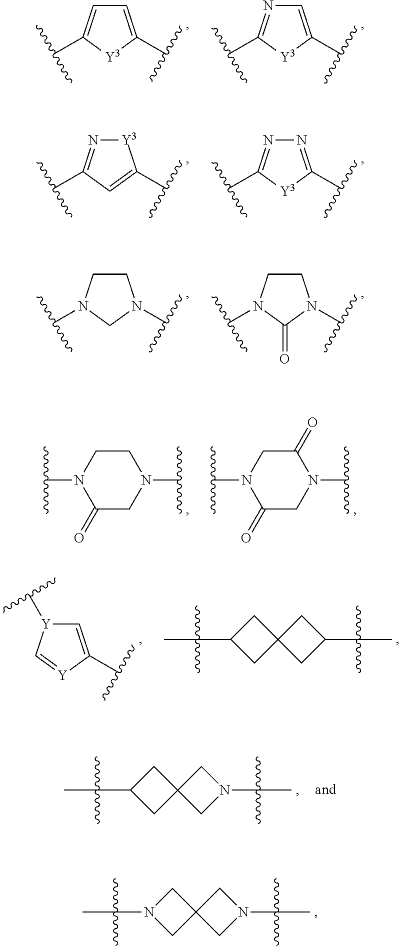

wherein the rings in B are unsubstituted or substituted with one or more substituents selected from alkyl, haloalkyl, halogen, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo;

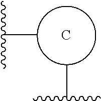

is selected from

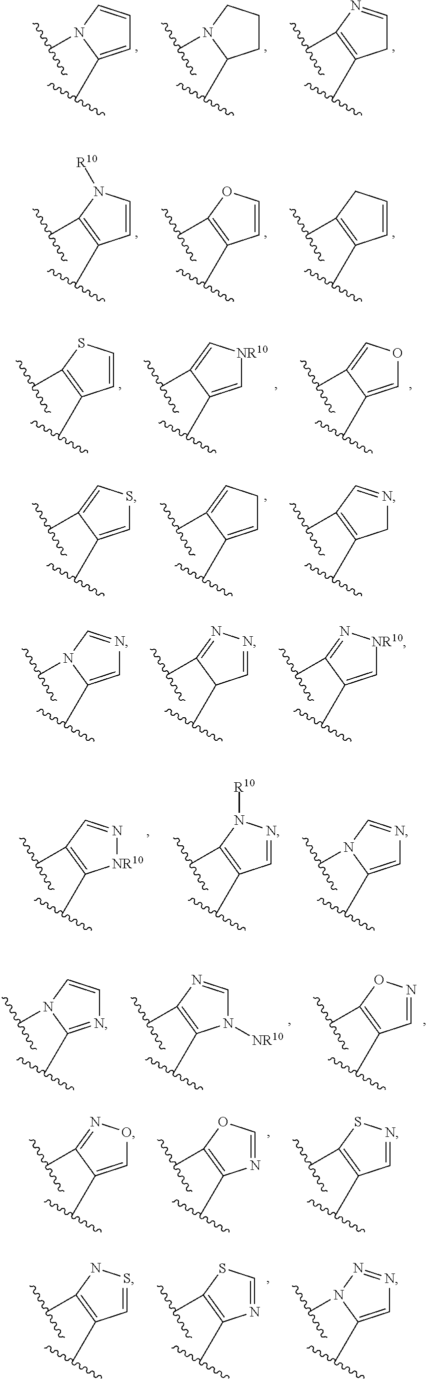

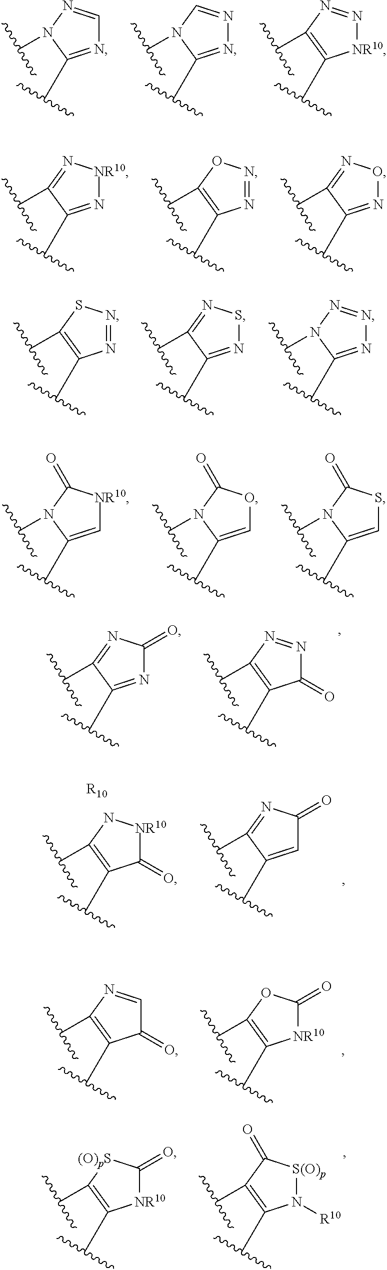

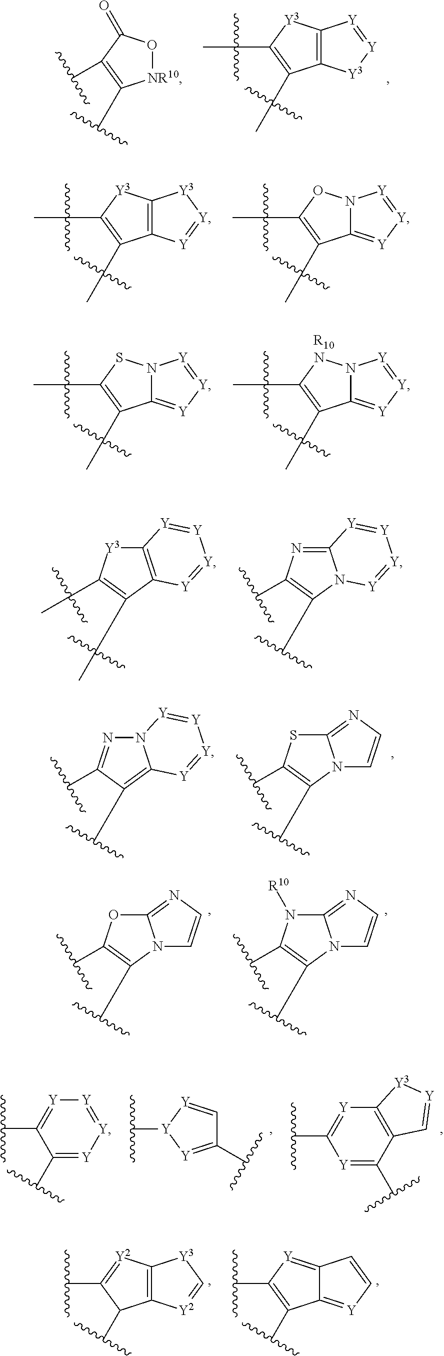

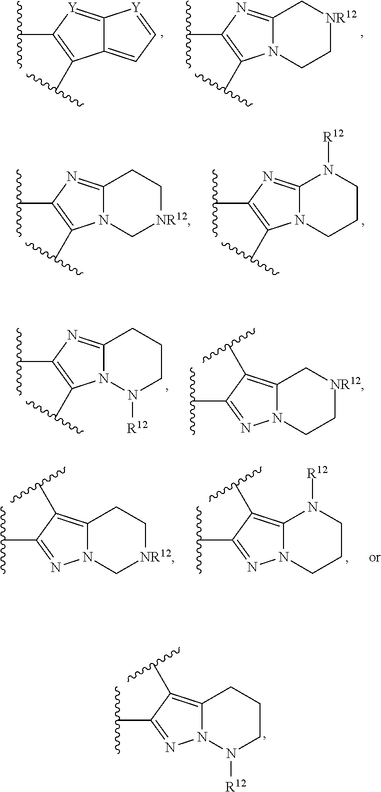

or optionally substituted variants thereof; Y² is selected from —CH═ or N; Y³ is selected from C(R⁶)₂, NR⁶, O, or S; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (IV) is also represented by Formula (IVa):

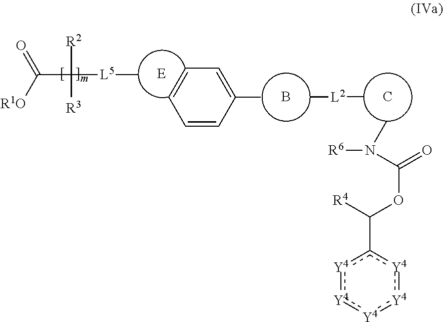

wherein

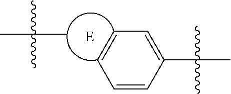

is selected from

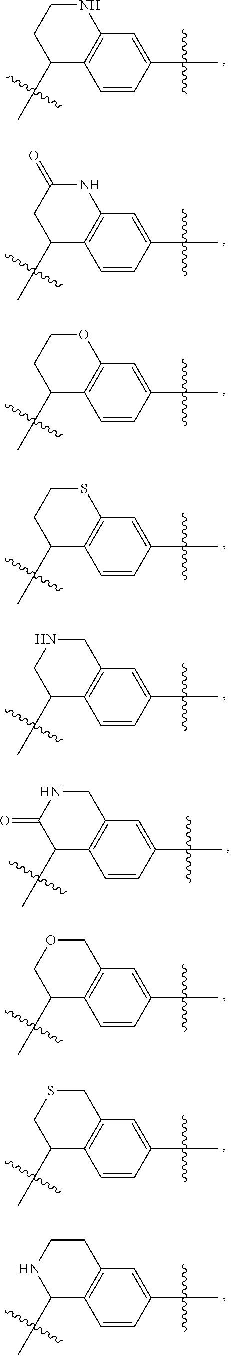

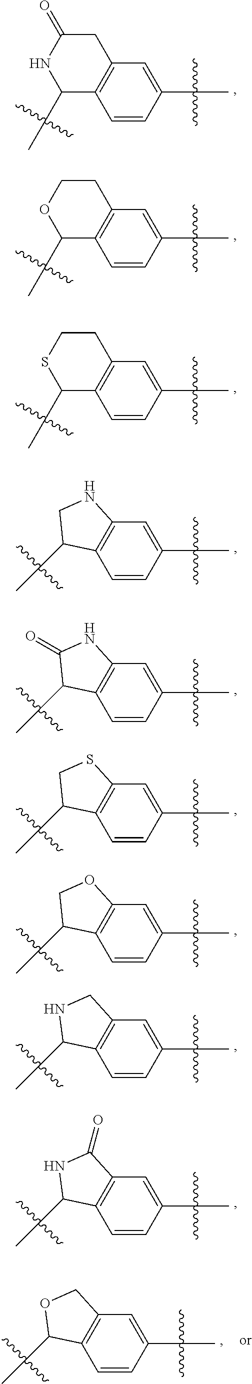

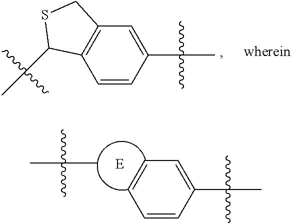

is unsubstituted or substituted with one or more substituents selected from alkyl, halogen, haloalkyl, hydroxy, alkoxy, haloalkoxy, cyano, or oxo.

In some embodiments, D is selected from

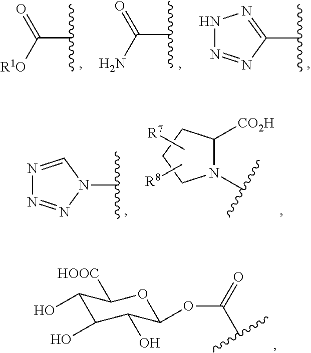

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; and each R¹² is independently selected from hydrogen, alkyl, acyl, C-carboxy, C-amido, sulfinyl, sulfonyl, or S-sulfonamido.

In some embodiments, B is selected from phenyl or naphthyl, and wherein B is unsubstituted or substituted with one or more substituents selected from alkyl, halogen, haloalkyl, hydroxy, alkoxy, haloalkoxy, cyano, or oxo. In some of such embodiments, B is unsubstituted phenyl. In some other such embodiments, B is a phenyl substituted with one or more halogen.

In some embodiments,

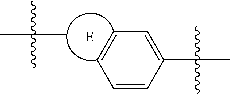

is selected from

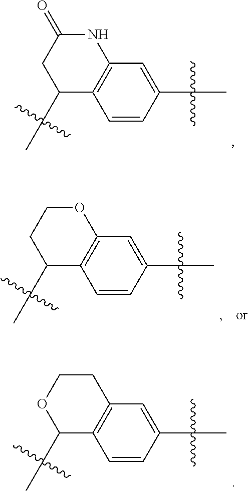 [SMILES: *c1ccc2c(c1)NC(=O)CC2* ; *c1ccc2c(c1)OCCC2* ; *c1ccc2c(c1)CCOC2*]

Some embodiments described herein of the compound of Formula (IV) or (IVa), rings in B and

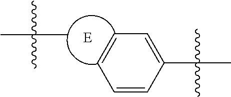

are unsubstituted.

Some embodiments described herein of the compound of Formula (IV) or (IVa), rings in B and

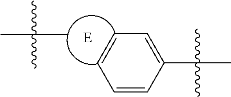

are substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo; or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

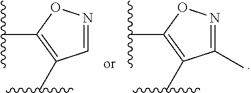

In some embodiments, C is selected from

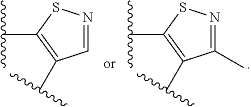

In some embodiments, C is selected from

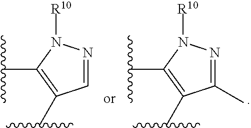

In some such embodiments, R¹⁰ is hydrogen. In some such embodiments, R¹⁰ is C₁₋₃ alkyl or C₃₋₆ cycloalkyl.

In some embodiments, C is

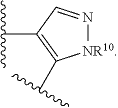

In some other embodiments, C is

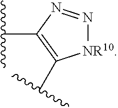

In some other embodiments, C is selected from

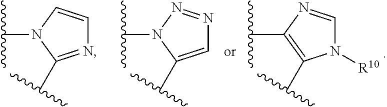

In some such embodiments, R¹⁰ is C₁₋₃ alkyl or C₃₋₆ cycloalkyl. In some such embodiments, R¹⁰ is hydrogen.

In some embodiments, C is selected from

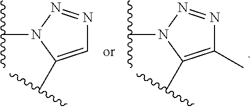

In some embodiments, C is selected from

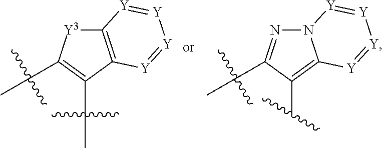

wherein

Y³ is selected from O or S. In some such embodiments, Y is a CR⁶. In some other such embodiments, at least one Y is nitrogen.

In some embodiments, C is selected from

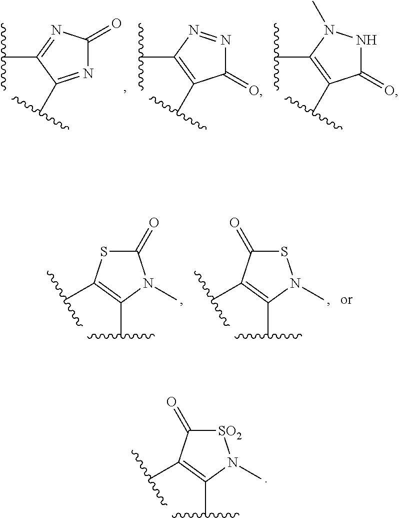

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is hydrogen. In some embodiments, R¹ is hydrogen.

R⁴ is selected from hydrogen or alkyl optionally substituted with halogen. In some other embodiments, R⁴ is unsubstituted alkyl.

Some embodiments of the compound of Formula (IV) or (IVa),

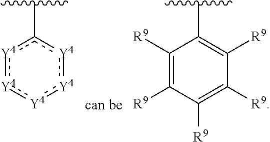

In some such embodiments, each of R⁹ is hydrogen. In some other such embodiments, at least one R⁹ is selected from C₁₋₃ alkyl or halogen.

In some embodiments,

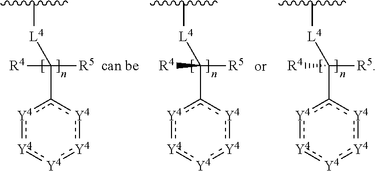

Some embodiments of the compound of Formula (IV) are selected from compounds of Table 4 as shown below, and pharmaceutically acceptable salt thereof.

Some embodiments of the compound of Formula (IV) are selected from compounds IT011, IT012 or IT037, as shown in Table 12.

Formula V

Some embodiments disclosed herein include a compound of Formula (V) as described above or a pharmaceutically acceptable salt thereof.

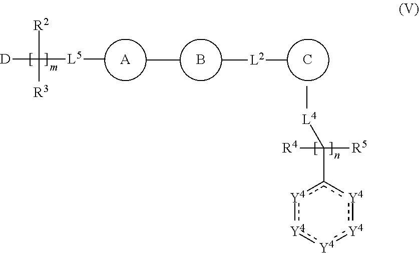

In some embodiments,

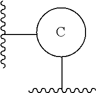

is selected from

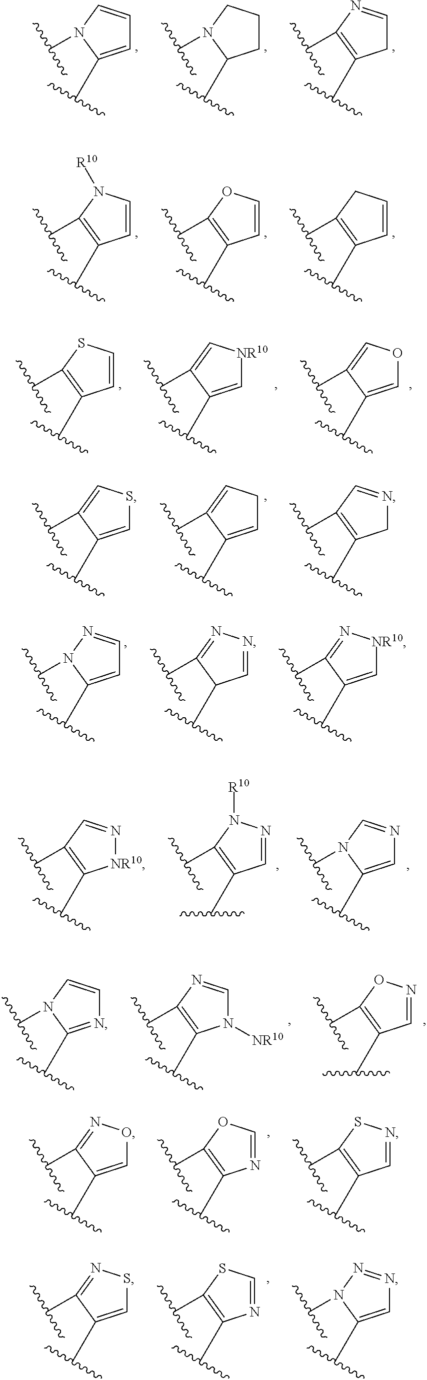

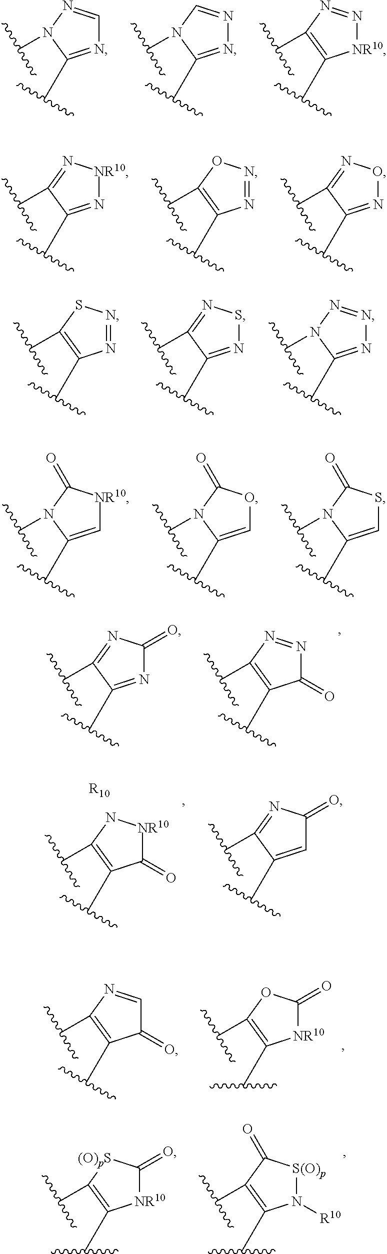

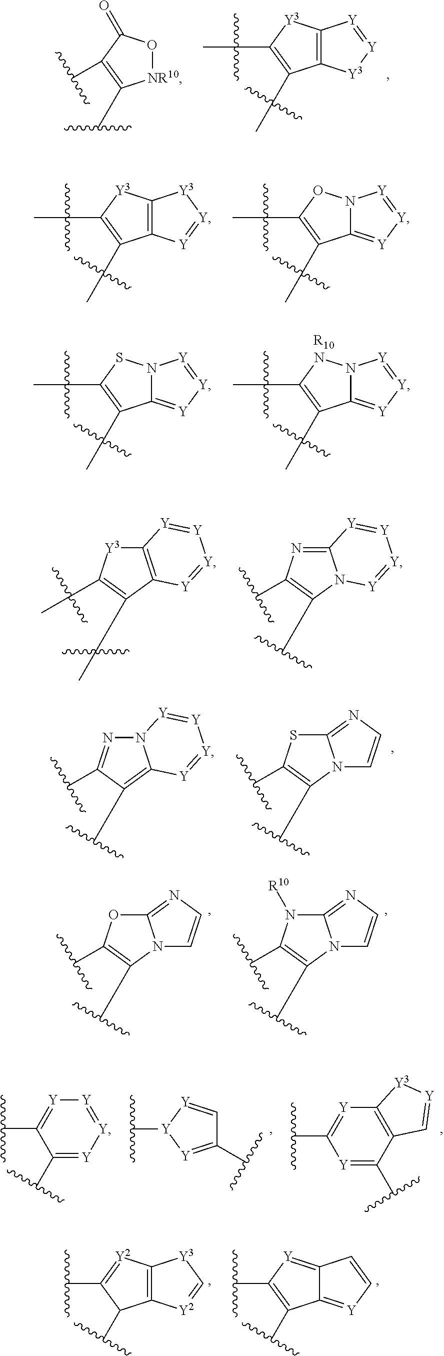

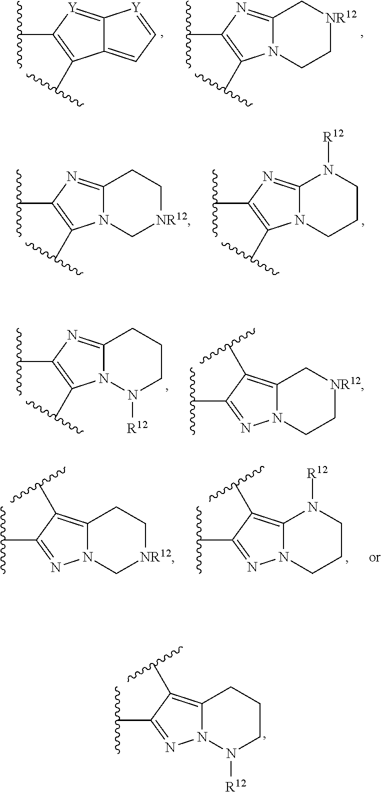

or optionally substituted variants thereof; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (V) is also represented by Formula (Va):

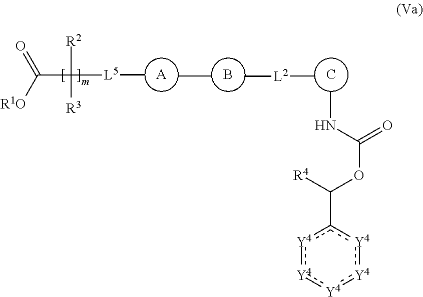

wherein one of A or B is an acetylene and the other one of A or B is selected from

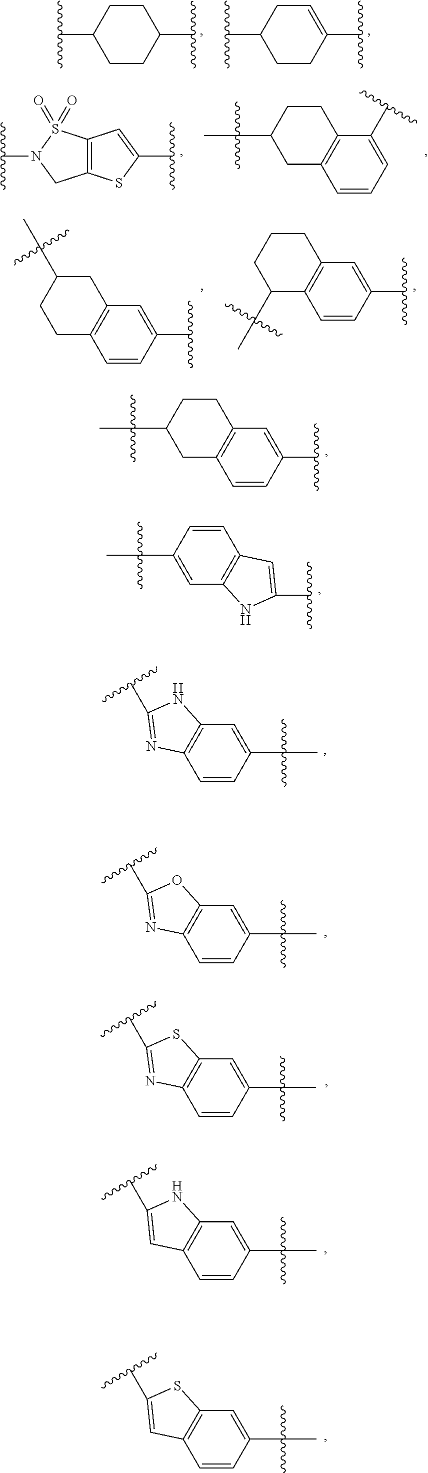

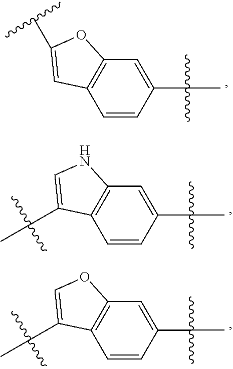

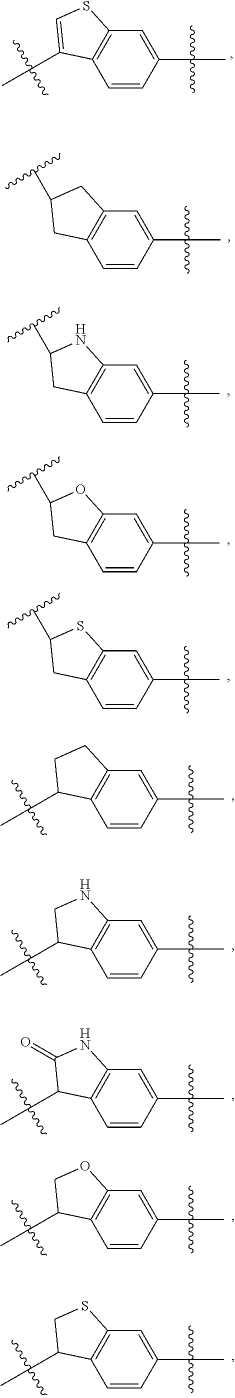

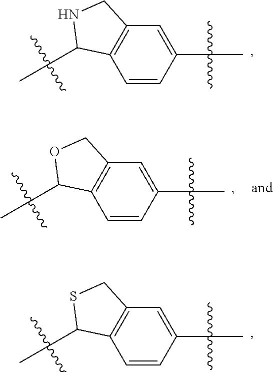 [SMILES: *c1ccc2c(c1)CNC2* ; *c1ccc2c(c1)COC2* ; *c1ccc2c(c1)CSC2*]

wherein rings in A or B is unsubstituted or substituted with one or more substituents selected from alkyl, halogen, haloalkyl, hydroxy, alkoxy, haloalkoxy, cyano, or oxo. In some such embodiments, one of A or B is an acetylene and the other one of A or B is selected from

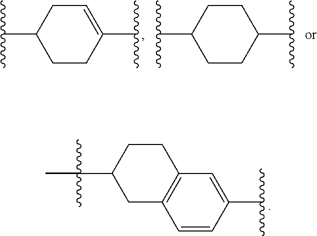

In some such embodiments, A is an acetylene. In some other such embodiments, B is an acetylene.

In some embodiments, one of A or B is an acetylene and the other A or B is a ring system selected from

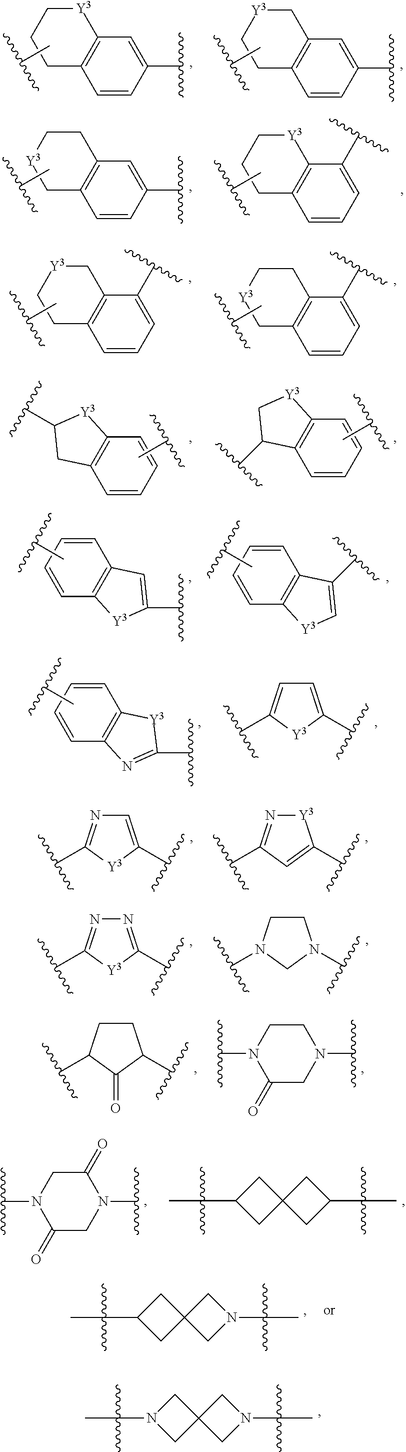

wherein A or B is unsubstituted or substituted with one or more substituents selected from alkyl, halogen, haloalkyl, hydroxy alkoxy haloalkoxy, cyano, or oxo: D is selected from

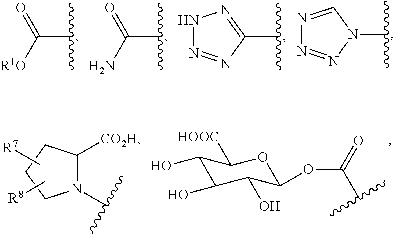

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; and each R¹² is independently selected from hydrogen, alkyl, acyl, C-carboxy, C-amido, sulfinyl, sulfonyl, or S-sulfonamido.

Some embodiments described herein of the compound of Formula (V) or (Va), rings in A or B are unsubstituted.

Some embodiments described herein of the compound of Formula (V) or (Va), rings in A or B is substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo; or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

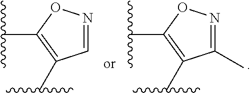

In some embodiments, C is selected from

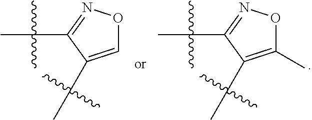

In some embodiments, C is selected from

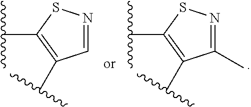

In some embodiments, C is selected from

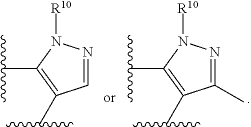

In some such embodiments, R¹⁰ is hydrogen. In some such embodiments, R¹⁰ is C₁₋₃ alkyl or C₃₋₆ cycloalkyl.

In some embodiments, C is

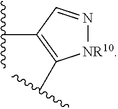

In some embodiments, C is

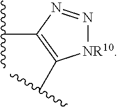

In some such embodiments, R¹⁰ is C₁₋₃ alkyl or C₃₋₆ cycloalkyl. In some such embodiments, R¹⁰ is hydrogen.

In some other embodiments, C is selected from

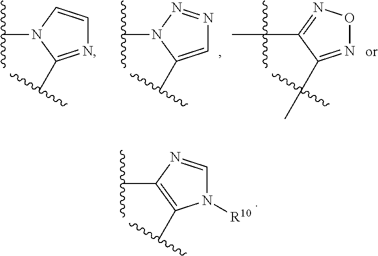

In some such embodiments, R¹⁰ is C₁₋₃ alkyl or C₃₋₆ cycloalkyl. In some such embodiments, R¹⁰ is hydrogen.

In some embodiments, C is selected from

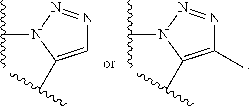

In some embodiments, C is selected from

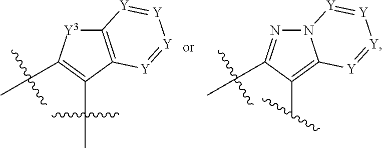

wherein Y³ is selected from O or S. In some such embodiments, Y is a CR⁶. In some other such embodiments, at least one Y is nitrogen.

In some embodiments, C is selected from

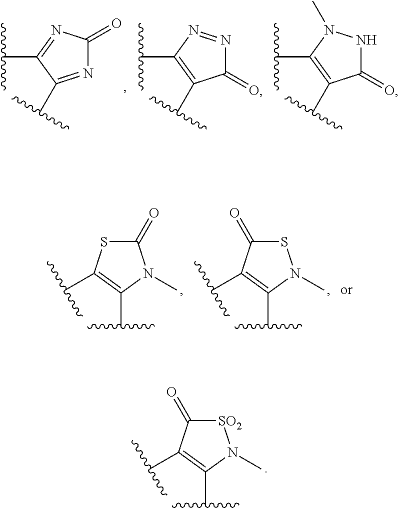

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is hydrogen. In some embodiments, R¹ is hydrogen.

In some embodiments, R⁴ is selected from hydrogen or alkyl optionally substituted with halogen. In some other embodiments, R⁴ is unsubstituted alkyl.

Some embodiments of the compound of Formula (V) or (Va),

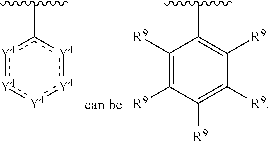

In some such embodiments, each of R⁹ is hydrogen. In some other such embodiments, at least one R⁹ is selected from C₁₋₃ alkyl or halogen.

In some embodiments,

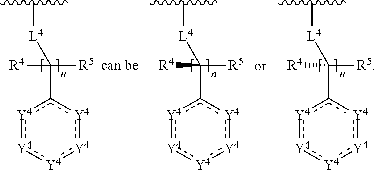

Some embodiments of the compound of Formula (V) are selected from compounds of Table 5 as shown below, and pharmaceutically acceptable salt thereof.

Some embodiments of the compound of Formula (V) are selected from compounds IT062, IT063 or IT092, as shown in Table 12.

Formula VI

Some embodiments disclosed herein include a compound of Formula (VI) as described above or a pharmaceutically acceptable salt thereof.

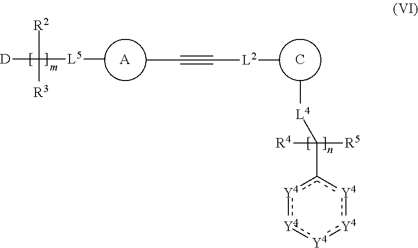

In some embodiments, A is selected from the group consisting of

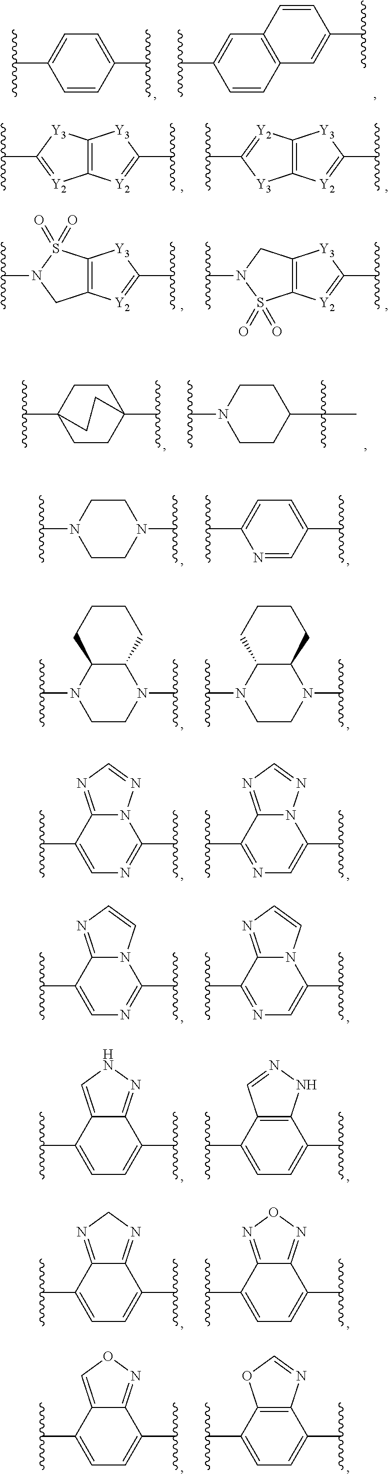

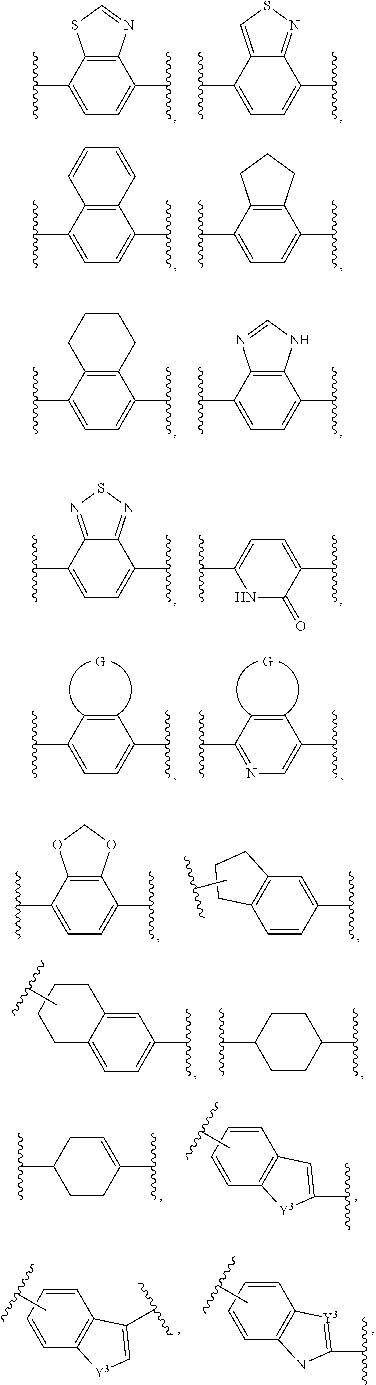

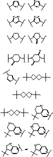

wherein the rings in A are unsubstituted or substituted with one or more substituents selected from alkyl, haloalkyl, halogen, hydroxy, alkoxy, haloalkoxy, cyano, or oxo;

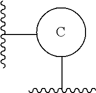

is selected from

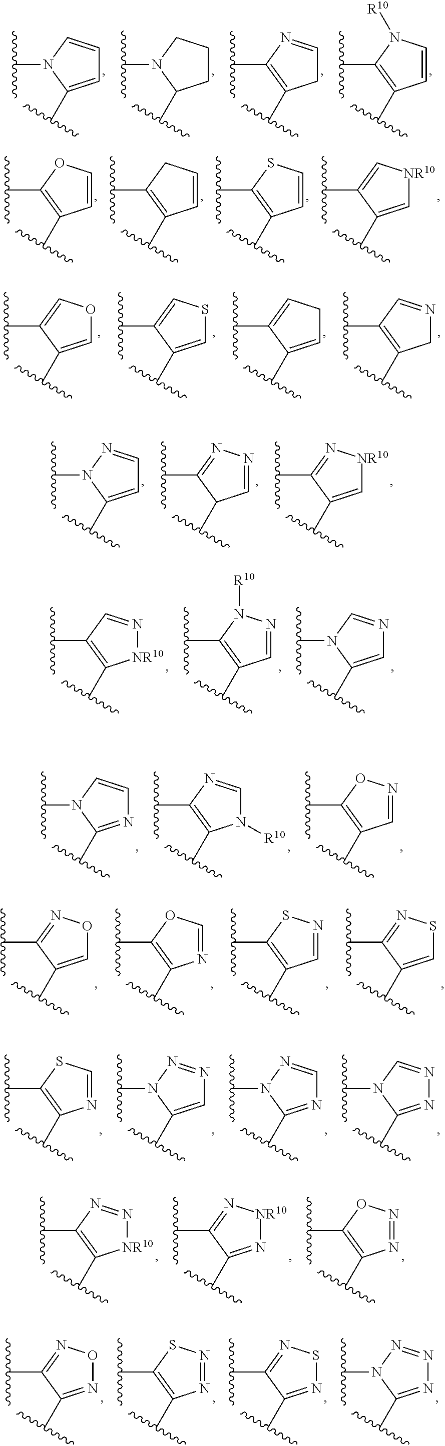

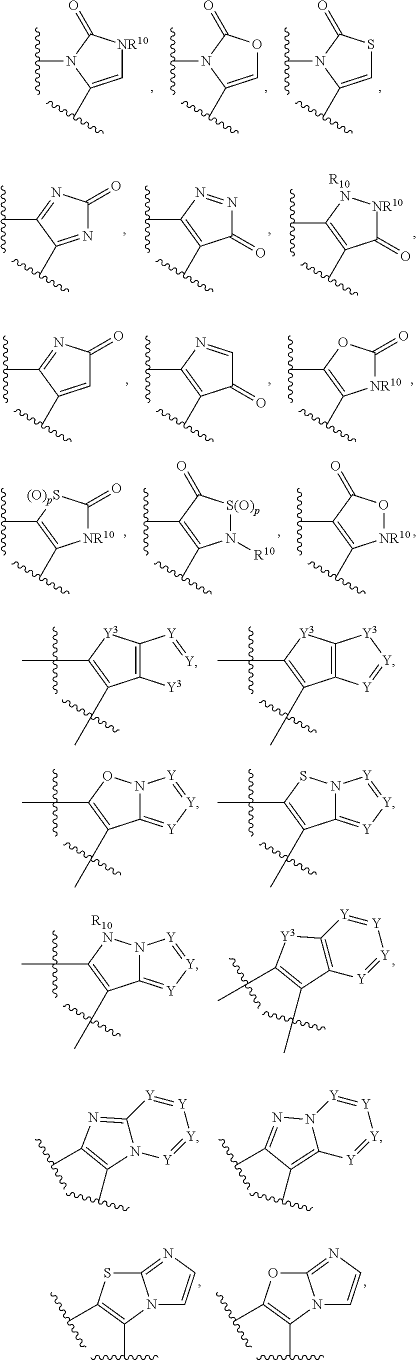

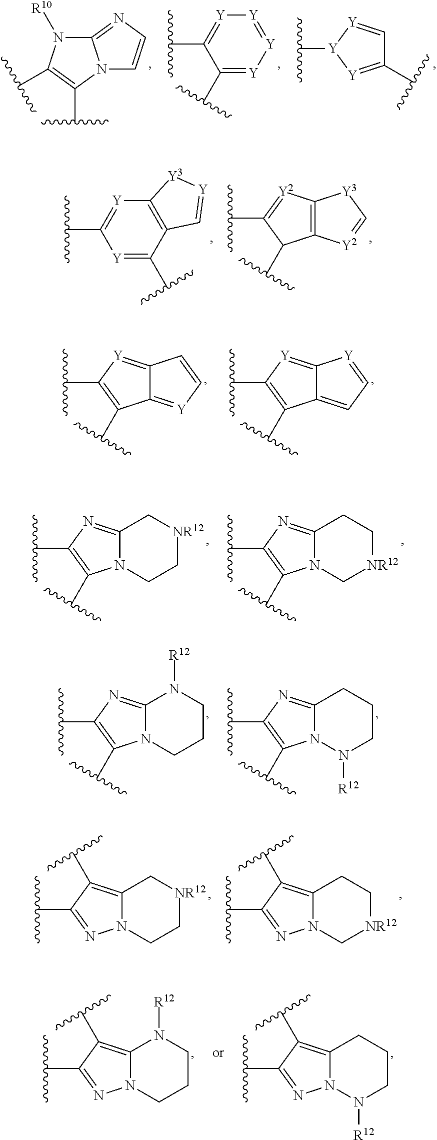

or optionally substituted variants thereof;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo;

Y² is selected from —CH═ or N; Y³ is selected from C(R⁶)₂, NR⁶, O or S; Y⁵ is selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (VI) is also represented by Formula (VIa):

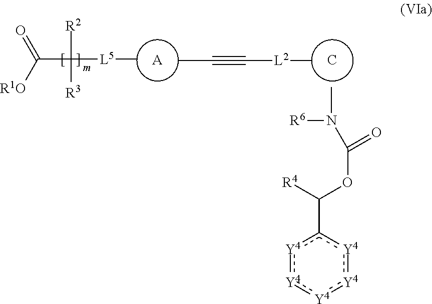

wherein A is selected from

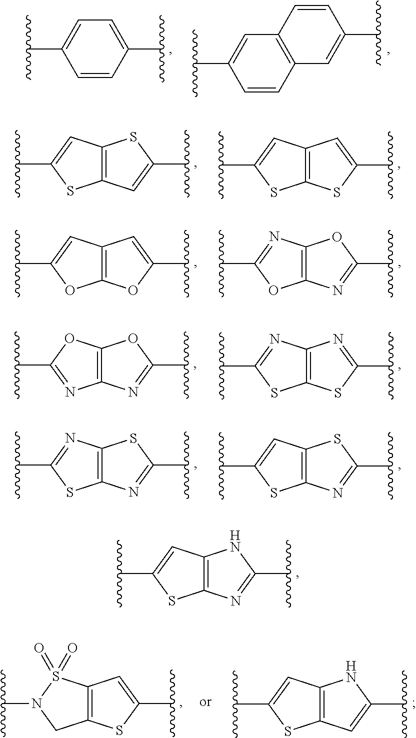

and wherein the rings in A are unsubstituted or substituted with one or more substituents selected from alkyl, haloalkyl, halogen, hydroxy, alkoxy, haloalkoxy, cyano, or oxo. In some further embodiments, A is phenyl. In some further embodiments, A is naphthyl.

In some embodiments, D is selected from

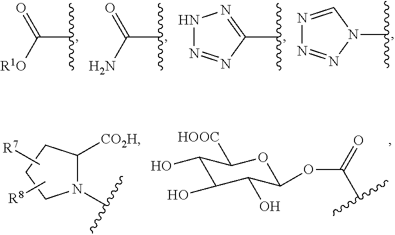

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; and each R¹² is independently selected from hydrogen, alkyl, acyl, C-carboxy, C-amido, sulfinyl, sulfonyl, or S-sulfonamido.

Some embodiments described herein of the compound of Formula (VI) or (VIa), rings in A are unsubstituted.

Some embodiments described herein of the compound of Formula (VI) or (VIa), rings in A are substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo; or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

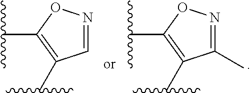

In some embodiments, C is selected from

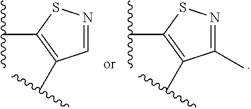

In some embodiments, C is selected from

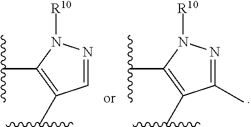

In some such embodiments, R¹⁰ is hydrogen. In some such embodiments, R¹⁰ is C₁₋₃ alkyl or C₃₋₆ cycloalkyl.

In some embodiments, C is

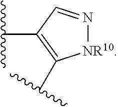

In some embodiments, C is

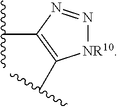

In some embodiments, C is selected from

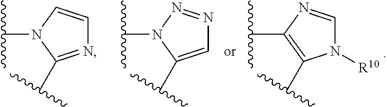

In some such embodiments, R¹⁰ is C₁₋₃ alkyl or C₃₋₆ cycloalkyl. In some such embodiments, R¹⁰ is hydrogen.

In some embodiments, C is selected from

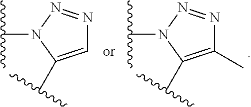

In some embodiments, C is selected from

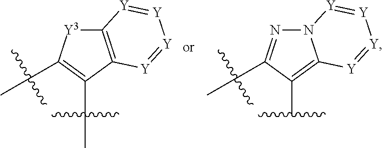

wherein Y³ is selected from O or S. In some such embodiments, Y is a CR⁶. In some other such embodiments, at least one Y is nitrogen.

In some embodiments, C is selected from

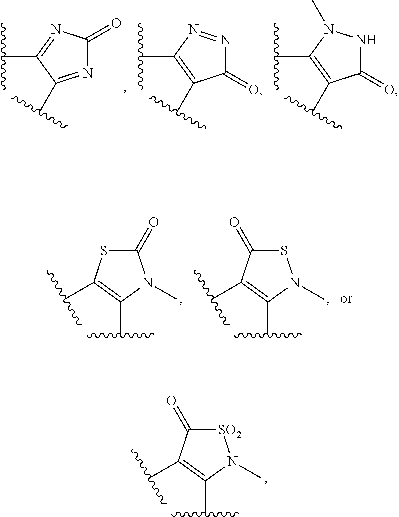

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a —CH═CH— linker. In some other embodiments, L⁵ is a —C≡C— linker.

In some embodiments, L² is a single bond.

In some embodiments, R¹ is hydrogen. In some embodiments, R⁶ is hydrogen.

In some embodiments, R⁴ is selected from hydrogen or alkyl optionally substituted with halogen. In some other embodiments, R⁴ is unsubstituted alkyl.

Some embodiments of the compound of Formula (VI) or (VIa),

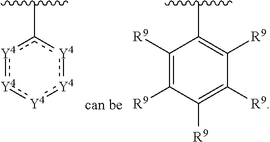

In some such embodiments, each of R⁹ is hydrogen. In some other such embodiments, at least one R⁹ is selected from C₁₋₃ alkyl or halogen.

In some embodiments,

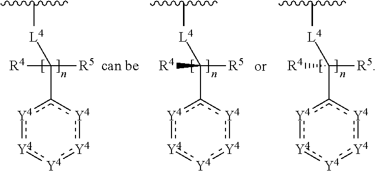

Some embodiments of the compound of Formula (VI) are selected from compounds of Table 6 as shown below, and pharmaceutically acceptable salt thereof.

Some embodiments of the compound of Formula (VI) are selected from compound IT013, as shown in Table 12.

Formula VII

Some embodiments disclosed herein include a compound of Formula (VII) as described above or a pharmaceutically acceptable salt thereof.

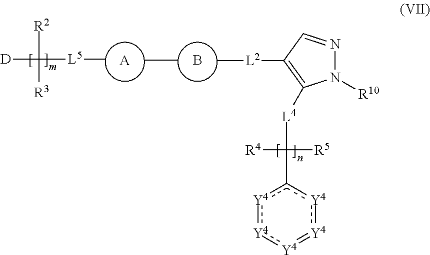

In some embodiments, one of A or B is an acetylene and the other one of A or B is selected from the group consisting of

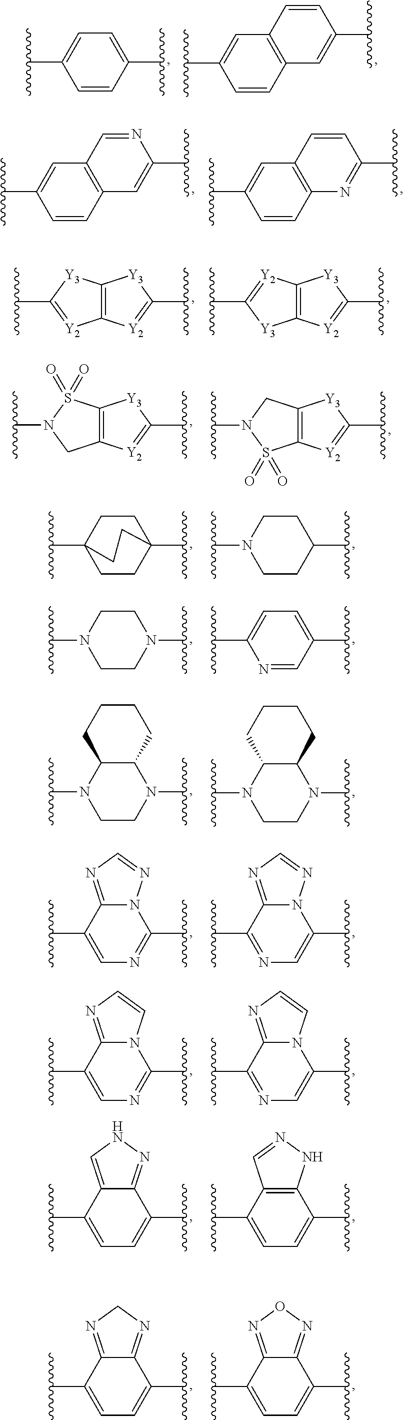

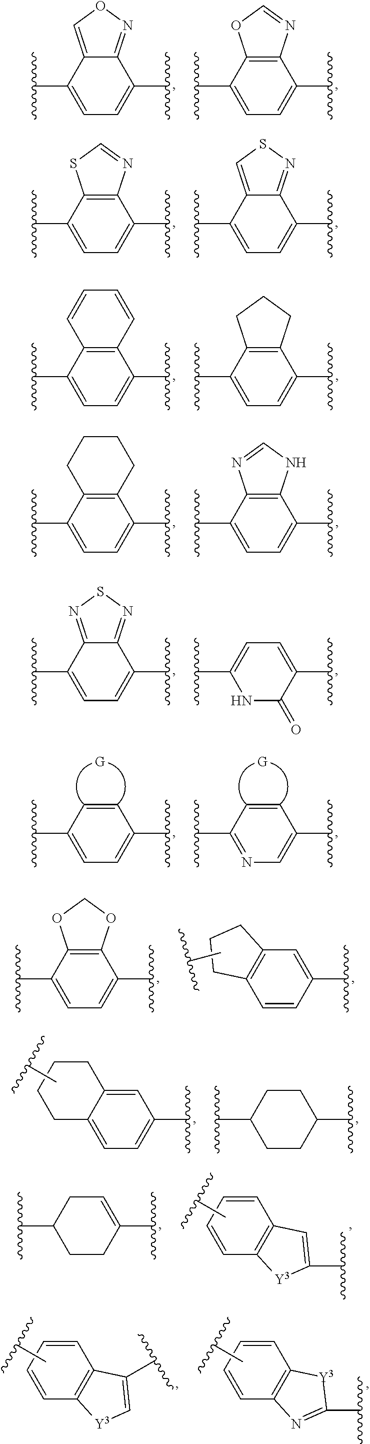

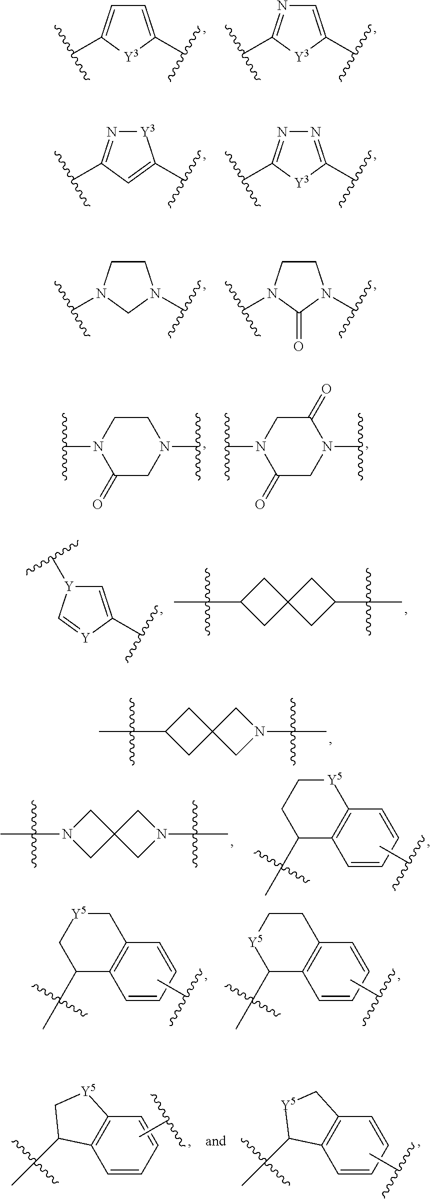

wherein the rings in A or B are unsubstituted or substituted with one or more substituents selected from alkyl, haloalkyl, halogen, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo;

each Y is independently selected from CR⁶ or N; Y² is selected from —CH═ or N; Y³ is selected from C(R⁶)₂, NR⁶, O or S; Y⁵ is selected from NR⁶, O or S; and D is selected from —OH,

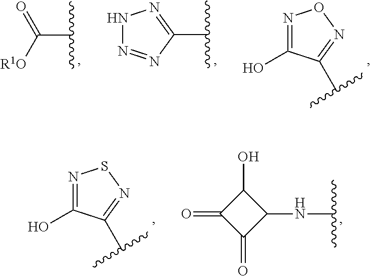

### —NHS(O)₂R¹⁴, or —C(O)—NHS(O)₂R¹⁴.

In some embodiments, the compound of Formula (VII) is also represented by Formula (VIIa):

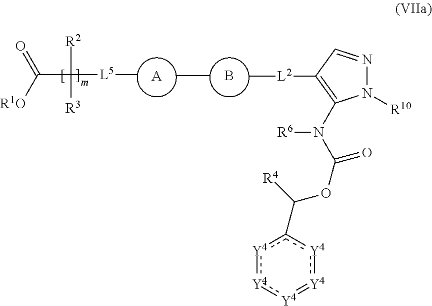

wherein one of A or B is an acetylene and the other one of A or B is selected from

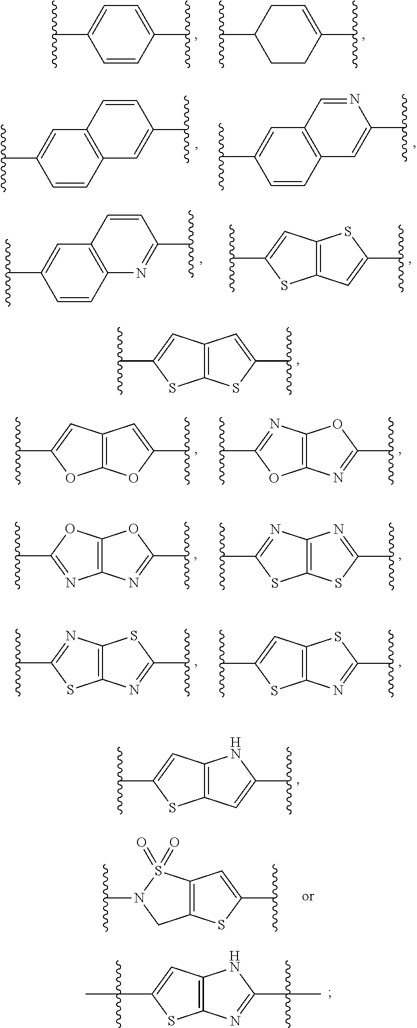

and wherein the rings in A or B are unsubstituted or substituted with one or more substituents selected from alkyl, haloalkyl, halogen, hydroxy, alkoxy, haloalkoxy, cyano, or oxo. In some further such embodiments, A is acetylene and B is phenyl. In some further such embodiments, A is acetylene and B is naphthyl. In some further such embodiments, A is acetylene and B is selected from

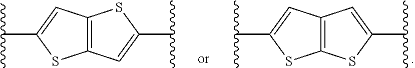 [SMILES: *c1cc2sc(*)cc2s1 ; *c1cc2cc(*)sc2s1]

In some further such embodiments, A is acetylene and B is selected from

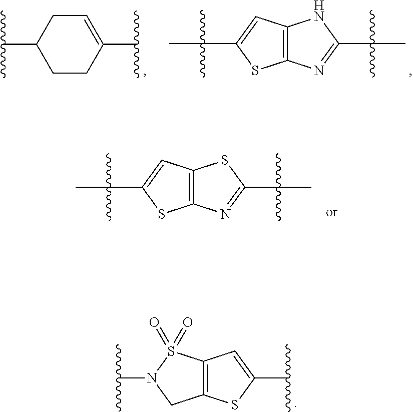 [SMILES: *C1=CCC(*)CC1 ; *c1nc2sc(*)cc2[nH]1 ; *c1cc2sc(*)nc2s1 ; *c1cc2c(s1)CN(*)S2(=O)=O]

In some further such embodiments, B is acetylene and A is phenyl. In some further such embodiments, B is acetylene and A is naphthyl. In some further such embodiments, B is acetylene and A is selected from

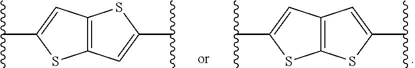 [SMILES: *c1cc2sc(*)cc2s1 ; *c1cc2cc(*)sc2s1]

In some further such embodiments, B is acetylene and A is selected from

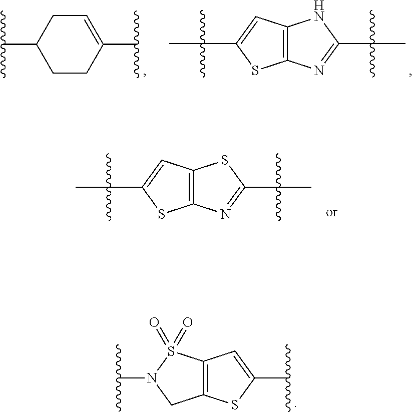 [SMILES: *C1=CCC(*)CC1 ; *c1nc2sc(*)cc2[nH]1 ; *c1cc2sc(*)nc2s1 ; *c1cc2c(s1)CN(*)S2(=O)=O]

In one embodiment, A is

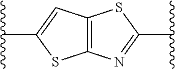 [SMILES: *c1cc2sc(*)nc2s1]

and B is acetylene. In another embodiment, A is

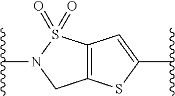 [SMILES: *c1cc2c(s1)CN(*)S2(=O)=O]

and B is acetylene.

In some alternative embodiments, L² is —(CH₂)₂—; B is absent; and A is selected from

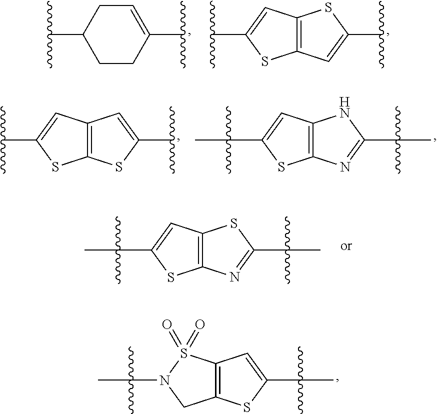

each optionally substituted with one or more substituents selected from the group consisting of alkyl, alkoxy, halogen, haloalkyl and cyano.

In some embodiments, D is selected from

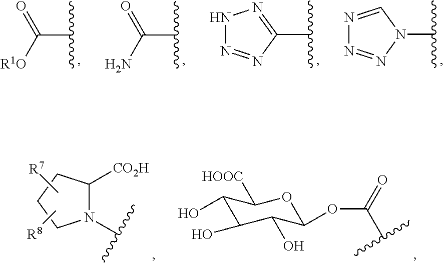

or carboxylic acid isosteres; L² is selected from a single bond, a —O— linker, a

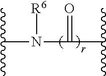

linker, a —C(O)— linker, a —CH₂— linker, a —CH₂O— linker, a —C≡C— linker, or a —CH═CH— linker; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; and each R¹⁰ is independently selected from hydrogen, alkyl, halogen, aryl, C₃₋₆ cycloalkyl, or cyano.

Some embodiments described herein of the compound of Formula (VII) or (VIIa), rings in A or B are unsubstituted.

Some embodiments described herein of the compound of Formula (VII) or (VIIa), rings in A or B are substituted with one or more substituents selected from alkyl, haloalkyl, halogen, hydroxy, alkoxy, haloalkoxy, cyano, or oxo.

In some embodiments, R¹⁰ is C₁₋₃ alkyl. In some other embodiments, R¹⁰ is C₃₋₆ cycloalkyl.

In some embodiments, R¹ is hydrogen or unsubstituted alkyl. In some other embodiments, R¹ is alkyl substituted with one or more substituents selected from the group consisting of alkoxy, C-amido, O-carboxy, and 6 membered heterocyclyl. In still some other embodiments, R¹ is optionally substituted aryl.

In some embodiments, m is 0. In some other embodiments, m is 1. In some other embodiments, m is 2.

In some embodiments, each of R² and R³ is hydrogen. In some other embodiments, one of R² and R³ is hydrogen and the other R² and R³ is aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is R⁶ⁱ s hydrogen. In some other embodiments, R is C₁₋₃ alkyl.

In some embodiments, R⁴ is alkyl optionally substituted with halogen. In some other embodiments, R⁴ is hydrogen.

Some embodiments of the compound of Formula (VII) or (VIIa),

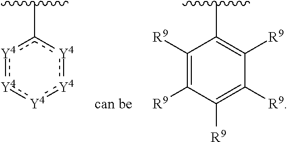

In some embodiments, each of R⁹ is hydrogen. In some other embodiments, at least one R⁹ is selected from C₁₋₃ alkyl or halogen.

In some embodiments,

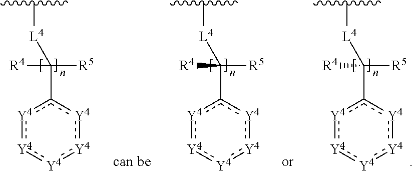

Some embodiments of the compound of Formula (VII) are selected from compounds of Table 7 as shown below, and pharmaceutically acceptable salt thereof.

Some embodiments of the compound of Formula (VII) are selected from compounds IT014-IT018, IT070, IT082-IT090, IT092, IT095, IT097-IT100 or IT103 as shown in Table 12.

Formula VIII

Some embodiments disclosed herein include a compound of Formula (VIII) as described above or a pharmaceutically acceptable salt thereof.

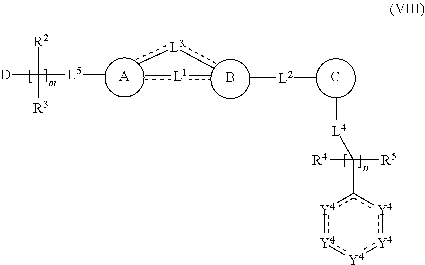

In some embodiments, each of A and B is selected from the group consisting of

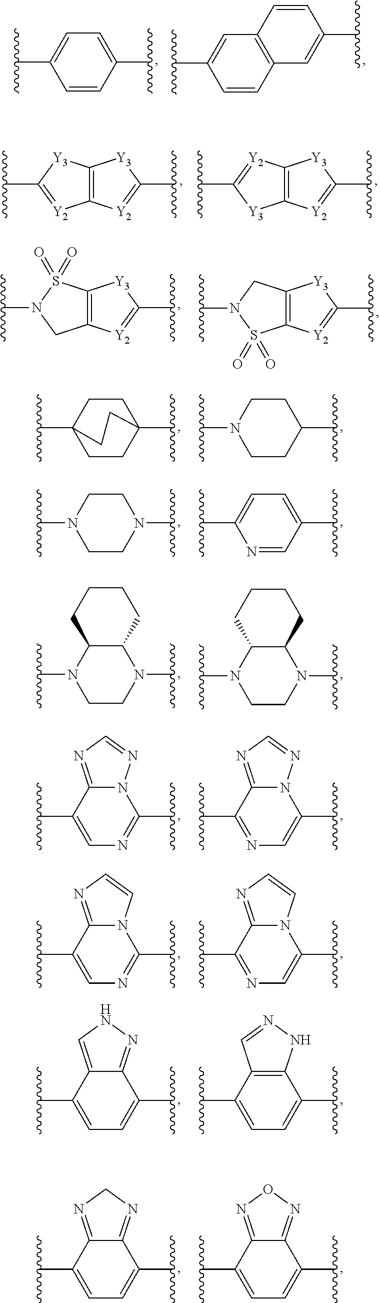

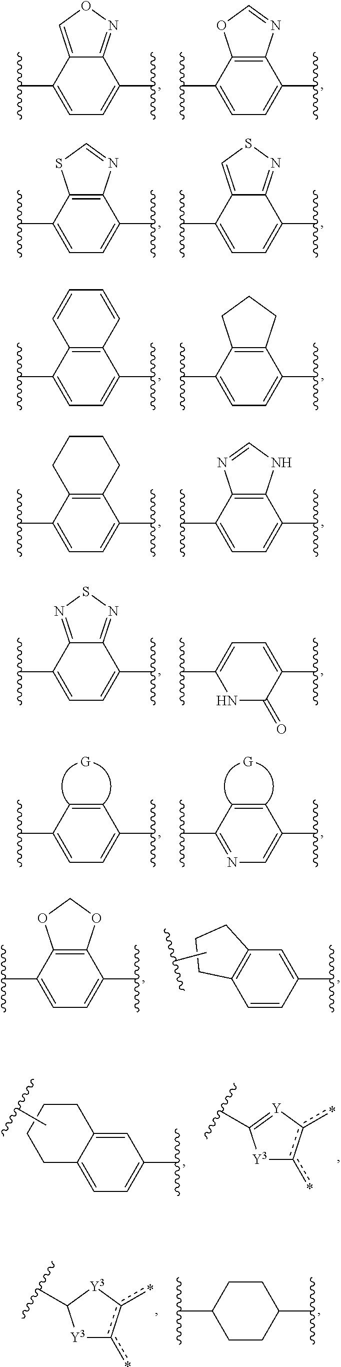

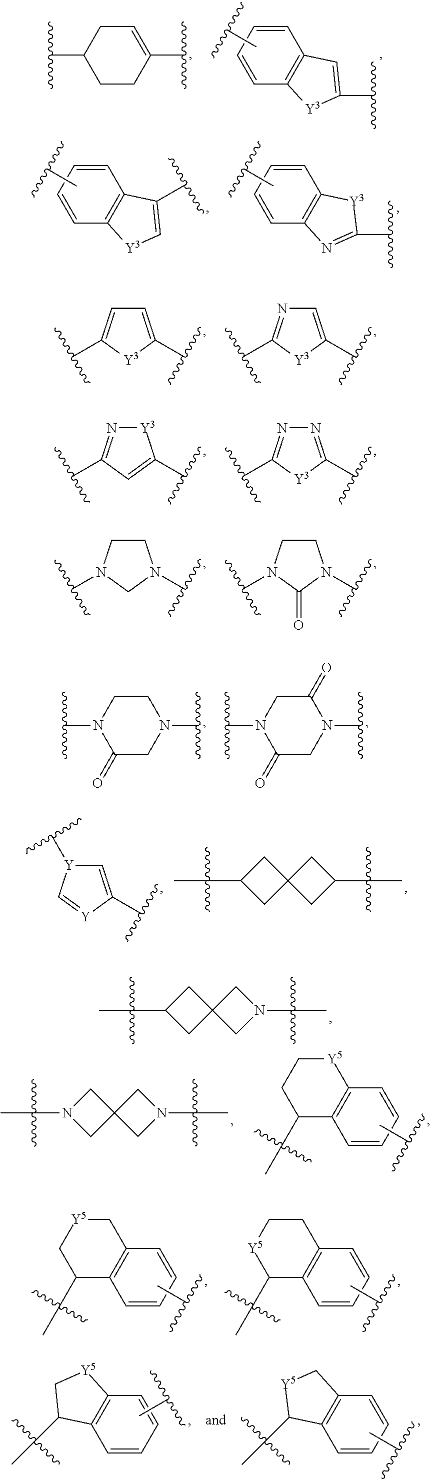

wherein each * is a point of attachment of A or B to L¹ or L³, and wherein the rings in A and B are unsubstituted or substituted with one or more substituents selected from alkyl, haloalkyl, halogen, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo; and Y⁵ is selected from NR⁶, O or S.

In some embodiments, the compound of Formula (VIII) is also represented by Formula (VIIIa):

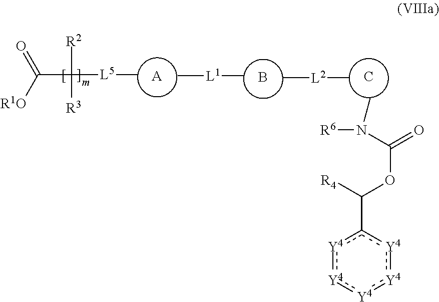

wherein one of A or B is phenyl and the other one of A or B is selected from

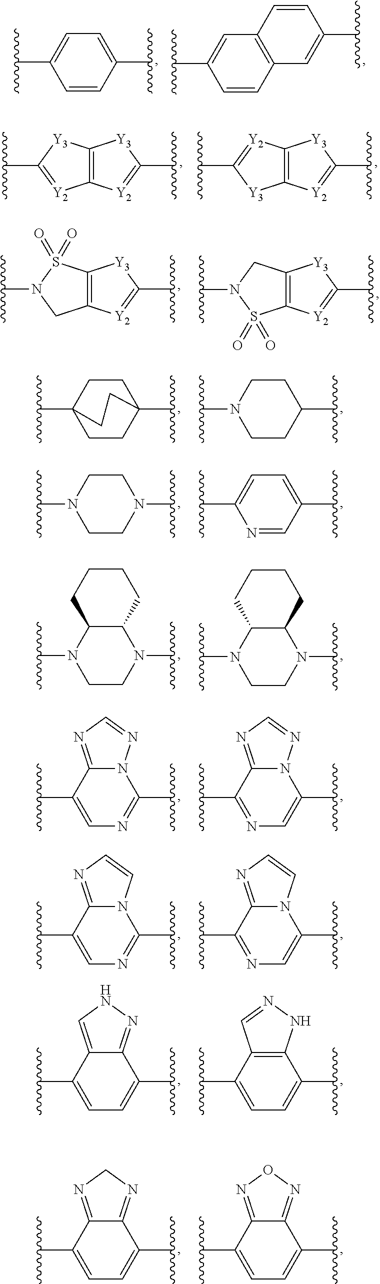

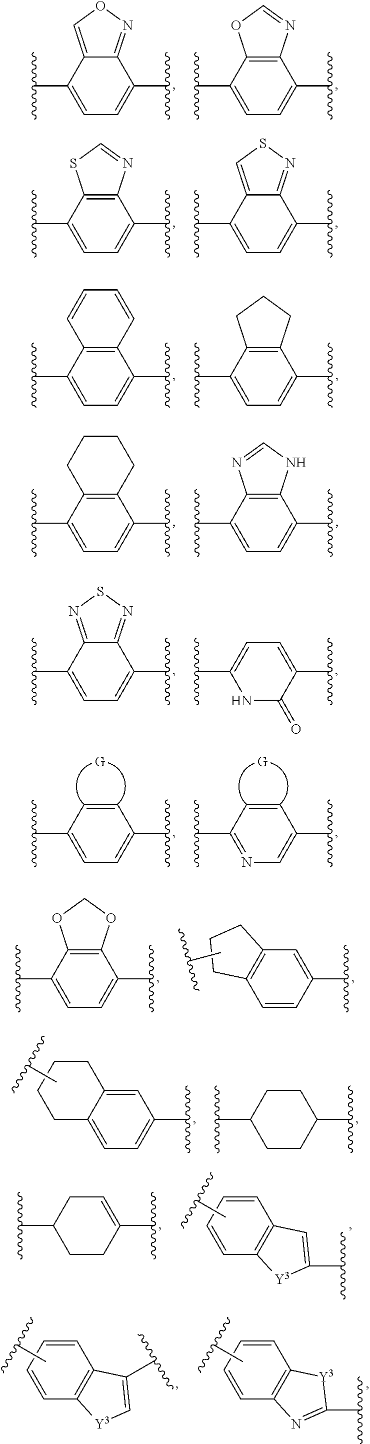

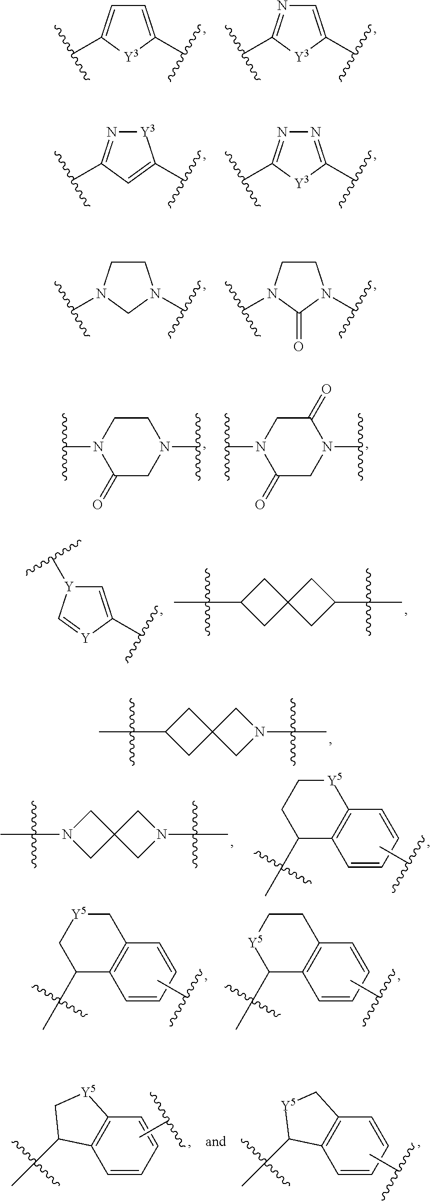

wherein each of A and B is unsubstituted or substituted with one or more substituents selected from alkyl, haloalkyl, halogen, hydroxy, alkoxy, haloalkoxy, cyano, or oxo. In some such embodiments, both A and B are phenyl. In some such embodiments, both A and B are unsubstituted phenyl.

In some embodiments,

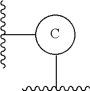

is a ring system selected from the group consisting of

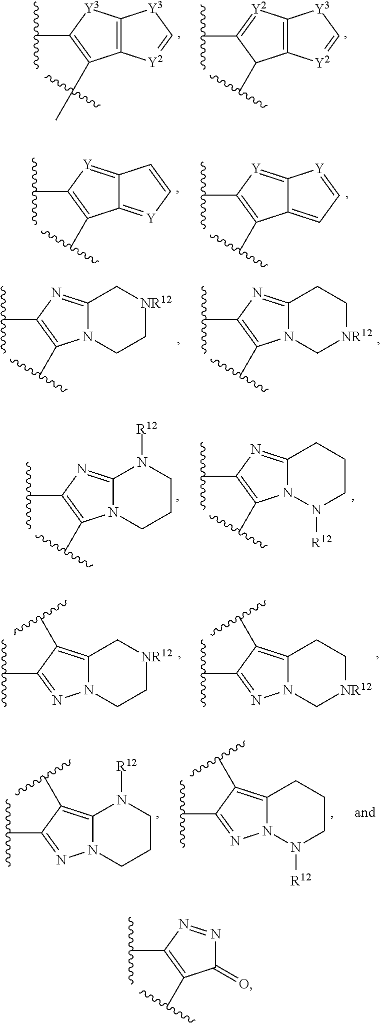

wherein C is optionally substituted; D is selected from

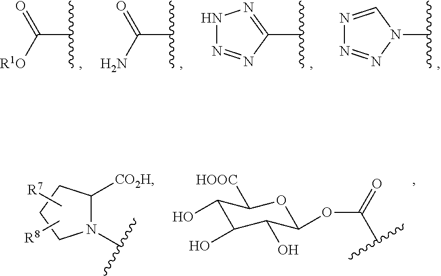

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; and each R¹² is independently selected from hydrogen, alkyl, acyl, C-carboxy, C-amido, sulfinyl, sulfonyl, or S-sulfonamido.

Some embodiments described herein of the compound of Formula (VIII) or (VIIIa), rings in A and B are unsubstituted.

Some embodiments described herein of the compound of Formula (VIII) or (VIIIa), rings in A and B are substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl, C₃₋₆ cycloalkyl, halogen, oxo or cyano. In some other embodiments, C is unsubstituted.

In some embodiments, C is selected from

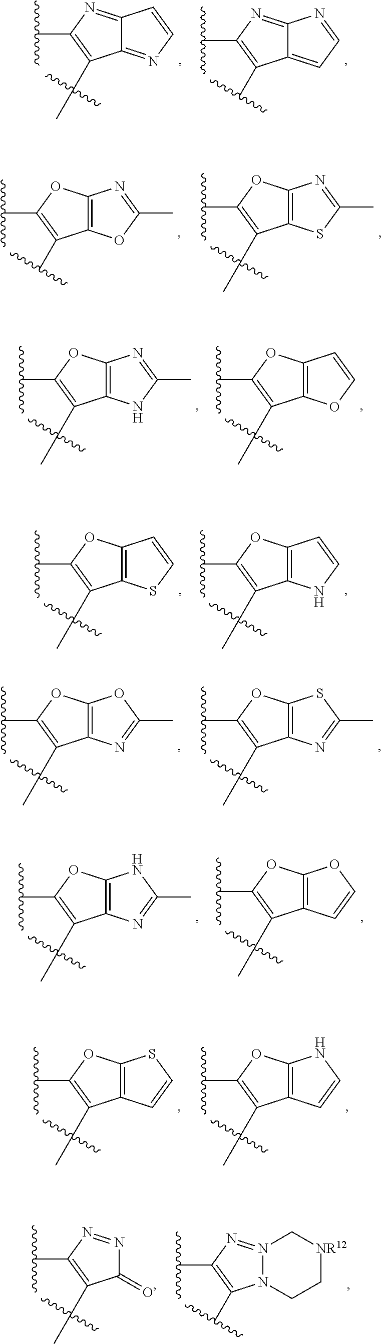

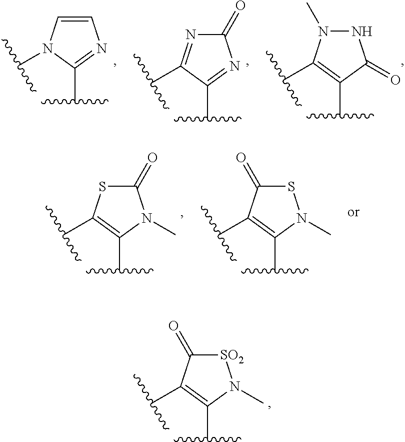

each optionally substituted with one or more substituents selected from the group consisting of C₁₋₃ alkyl optionally substituted with halogen or C₁₋₃ alkoxy; C₁₋₆ alkoxy; C₃₋₆ cycloalkyl; halogen; or cyano. In some such embodiment, C is

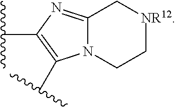

In some such embodiments, each R¹² is independently selected from hydrogen, C₁₋₃ alkyl, —C(O)CH₃, —S(O)₂CH₃, —C(O)NHCH₃, or —C(O)OC₂H₅. In some other such embodiment, C is selected from

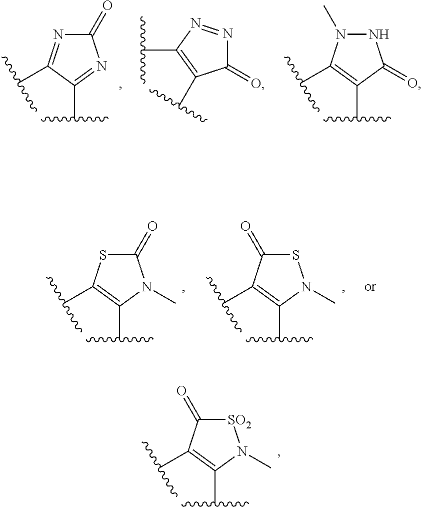

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond. In some embodiments, L¹ is a single bond.

In some embodiments, R⁶ is hydrogen. In some embodiments, R¹ is hydrogen.

In some embodiments, R⁴ is alkyl optionally substituted with halogen. In some other embodiments, R⁴ is hydrogen.

Some embodiments of the compound of Formula (VIII) or (VIIIa).

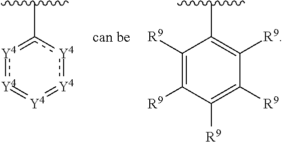

In some such embodiments, each of R⁹ is hydrogen. In some other such embodiments, at least one R⁹ is selected from C₁₋₃ alkyl or halogen.

In some embodiments,

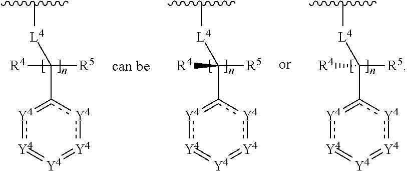

Some embodiments of the compound of Formula (VIII) are selected from compounds of Table 8 as shown below, and pharmaceutically acceptable salt thereof.

Some embodiments of the compound of Formula (VIII) are selected from compounds IT019-IT024, as shown in Table 12.

Formula IX

Some embodiments disclosed herein include a compound of Formula (IX) as described above or a pharmaceutically acceptable salt thereof.

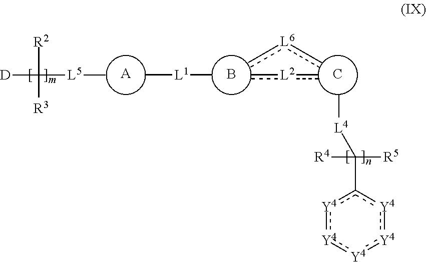

In some embodiments, A is acetylene or each of A and B is a ring system selected from

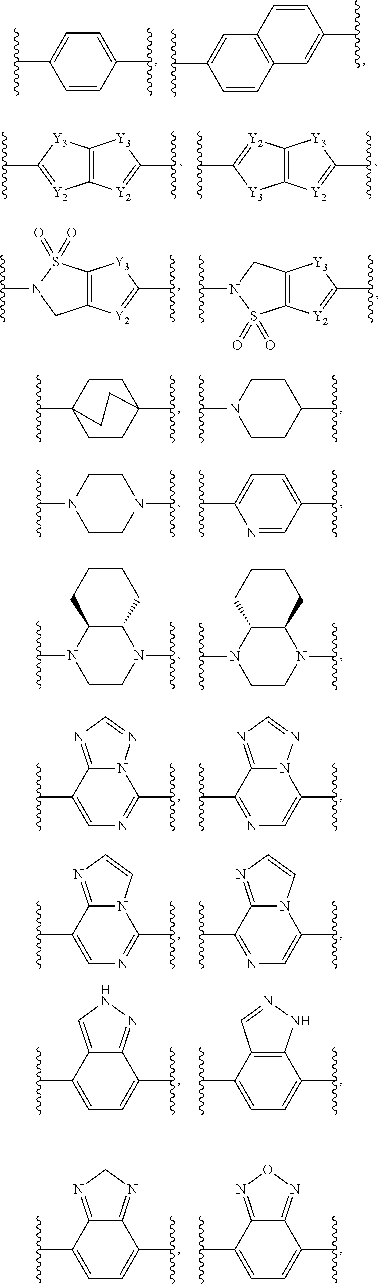

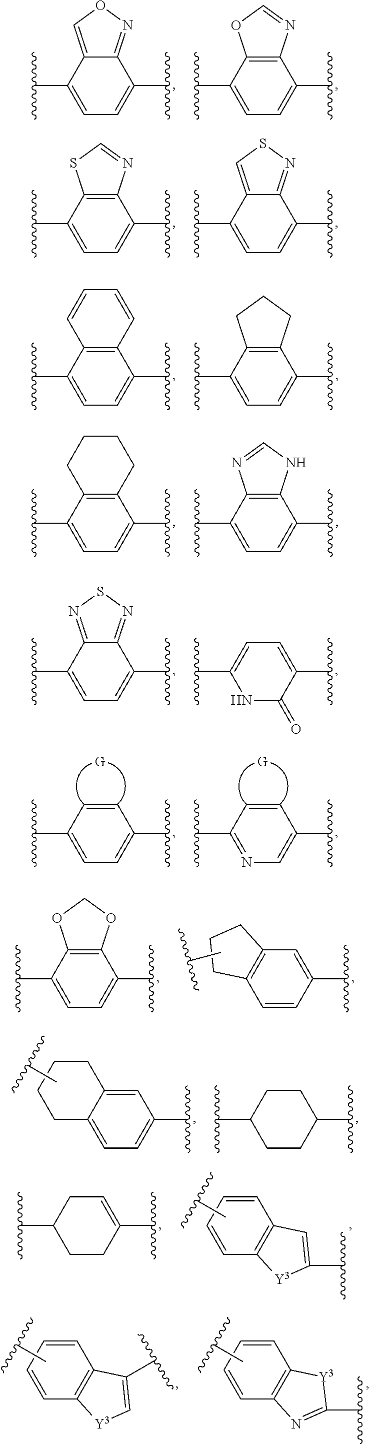

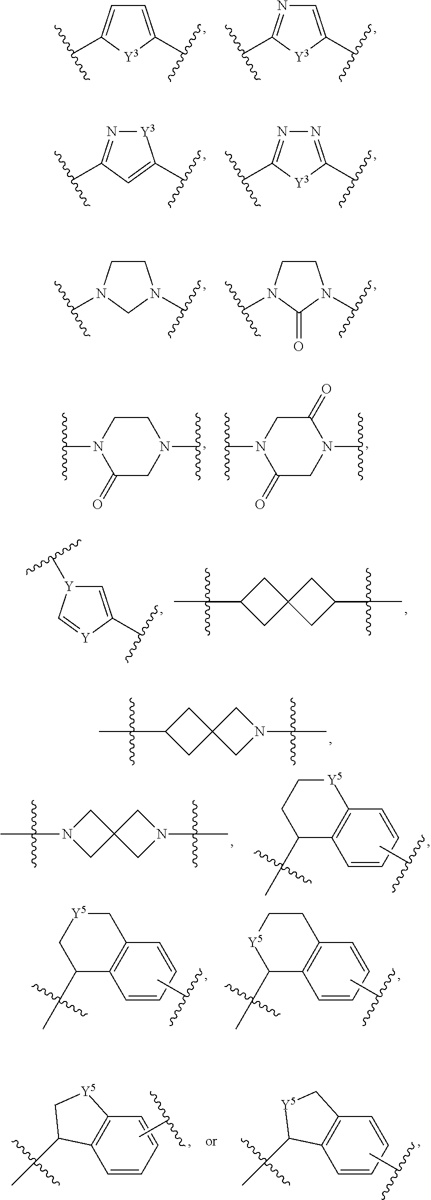

wherein each of A and B is unsubstituted or substituted with one or more substituents selected from alkyl, haloalkyl, halogen, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo;

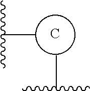

is selected from

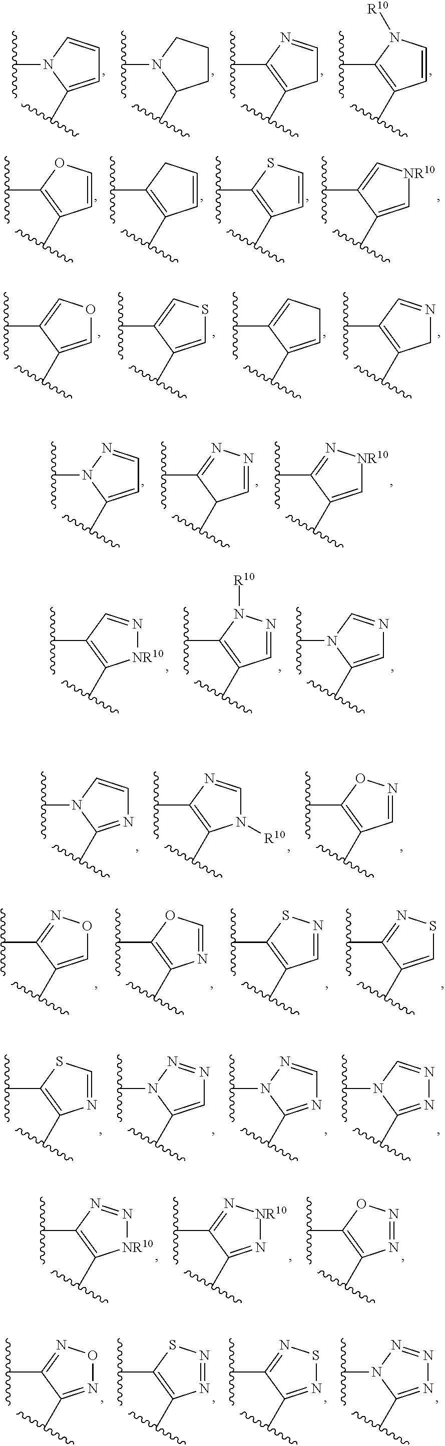

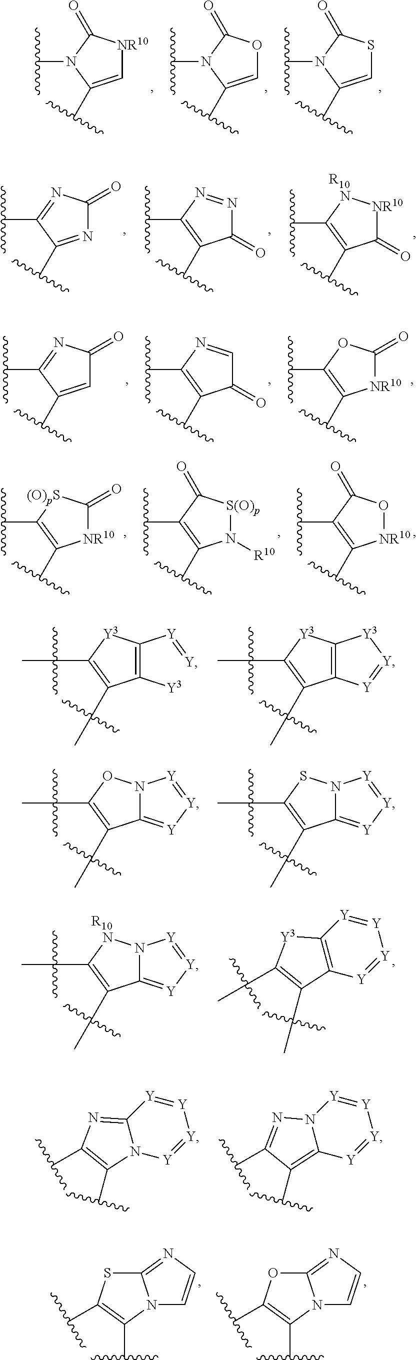

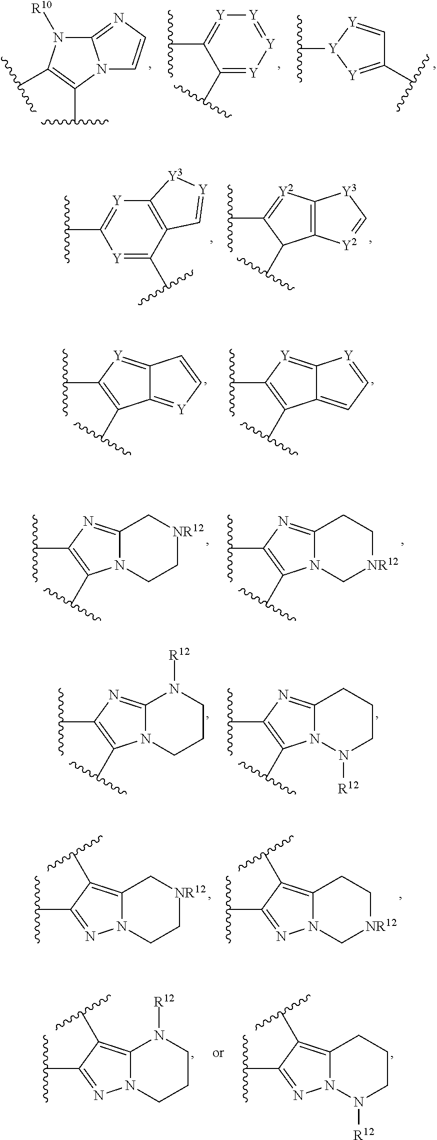

or optionally substituted variants thereof;

Y² is selected from —CH═ or N; Y³ is selected from C(R⁶)₂, NR⁶, O, or S; Y⁵ is selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (IX) is also represented by Formula (IXa):

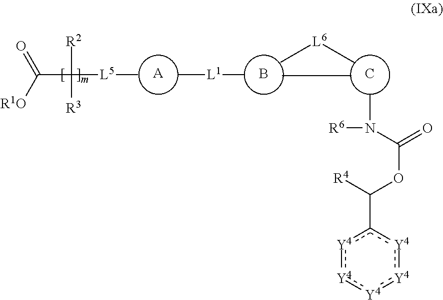

wherein B is phenyl; and A is selected from acetylene,

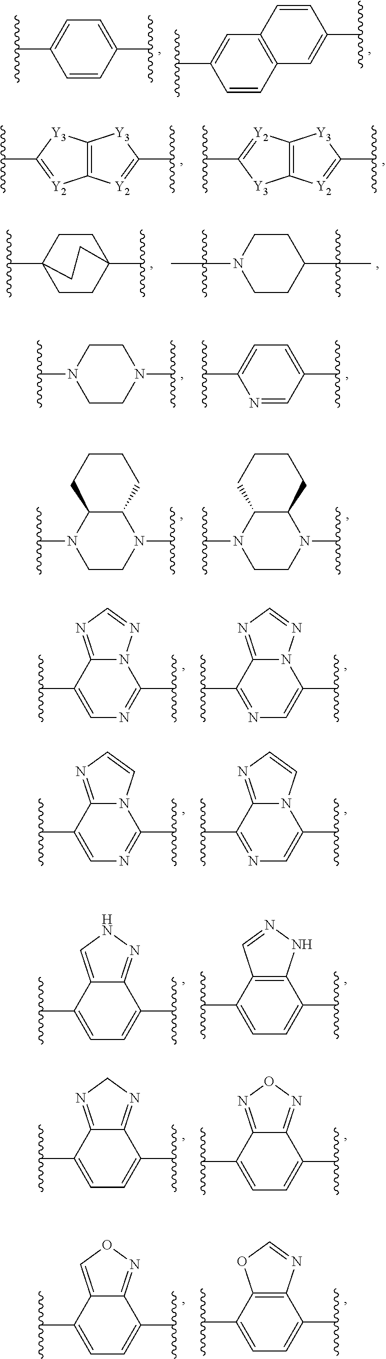

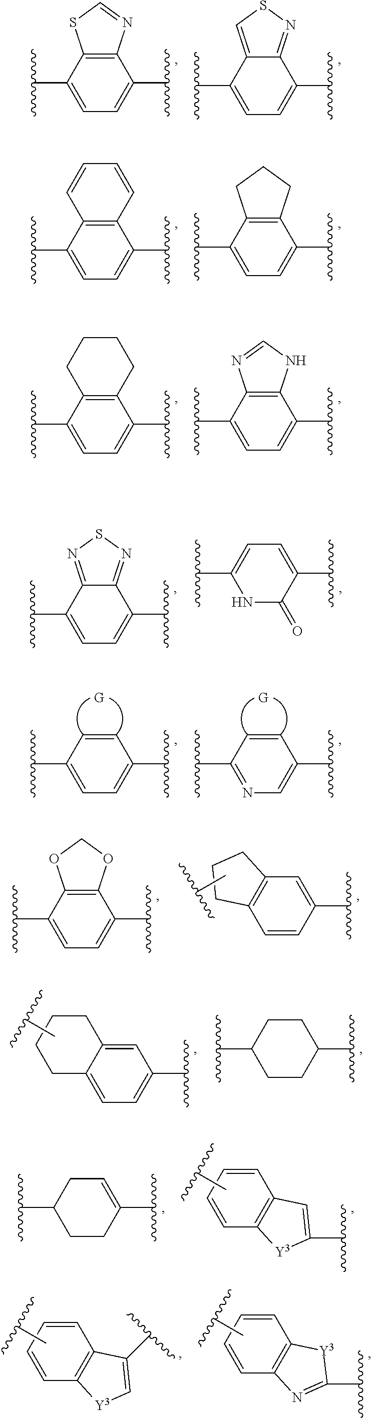

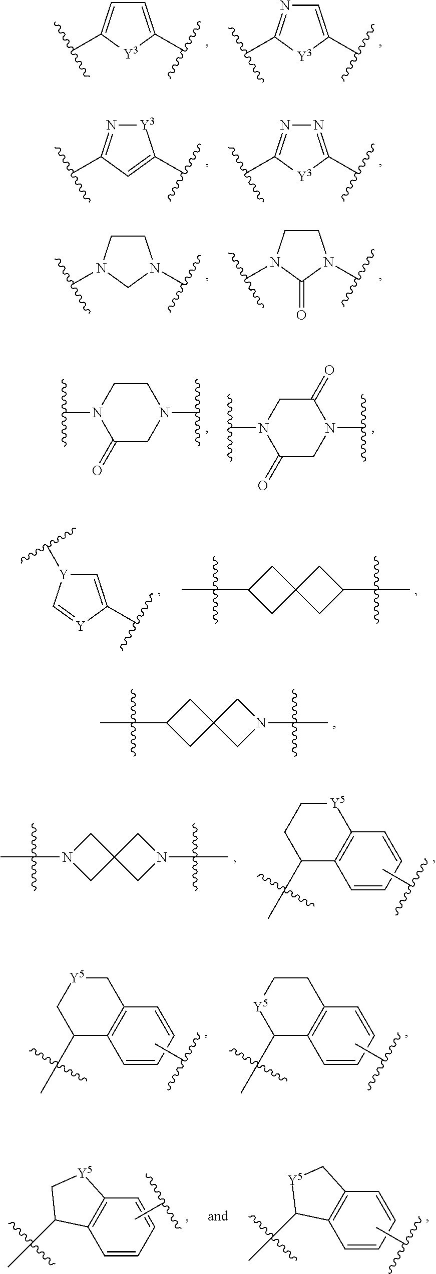

wherein each of the rings in A and B is unsubstituted or substituted with one or more substituents selected from alkyl, haloalkyl, halogen, hydroxy, alkoxy, haloalkoxy, cyano, or oxo. In some of such embodiments, both A and B are phenyl. In some of such embodiments, A is acetylene and B is phenyl.

In some embodiments, D is selected from

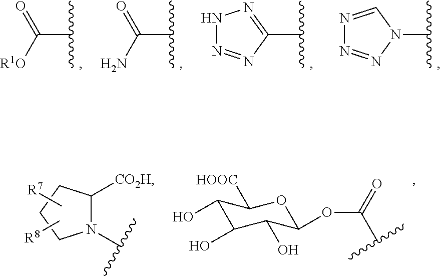

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; and each R¹² is independently selected from hydrogen, alkyl, acyl, C-carboxy, C-amido, sulfinyl, sulfonyl, or S-sulfonamido.

Some embodiments described herein of the compound of Formula (IX) or (IXa), rings in A and B are unsubstituted.

Some embodiments described herein of the compound of Formula (IX) or (IXa), rings in A and B are substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl alkyl optionally substituted with halogen or C₁₋₃ alkoxy; C₁₋₆ alkoxy; C₃₋₆ cycloalkyl; halogen; oxo; or cyano. In some other embodiments, C is unsubstituted.

In some further embodiments, C is selected from an oxazole, an isoxazole, a thiazole, or an isothiazole, and wherein C is unsubstituted or substituted with one or more substituents selected from C₁₋₃ alkyl alkyl optionally substituted with halogen or C₁₋₃ alkoxy; C₁₋₆ alkoxy; C₃₋₆ cycloalkyl; halogen or cyano.

In some embodiments, C is selected from

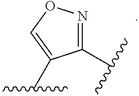 [SMILES: *c1conc1* ; "."]

In some embodiments, C is selected from

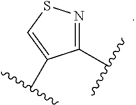 [SMILES: *c1csnc1* ; "."]

In some embodiments, C is

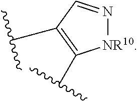

In some such embodiments, R¹⁰ is hydrogen. In some other such embodiments, R¹⁰ is C₁₋₃ alkyl or C₃₋₆ cycloalkyl.

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond. In some embodiments, L¹ is a single bond.

In some embodiments, L⁶ is

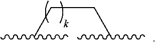

In some such embodiments, k is 0. In some other such embodiments, k is 1.

In some embodiments, L⁶ is

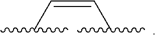 [SMILES: */C=C\* ; "."]

In some embodiments, R⁶ is hydrogen. In some embodiments, R¹ is hydrogen.

In some embodiments, R⁴ is alkyl.

Some embodiments of the compound of Formula (IX) or (IXa),

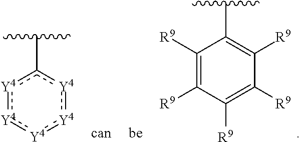

In some such embodiments, each of R⁹ is hydrogen. In some other such embodiments, at least one R⁹ is selected from C₁₋₃ alkyl or halogen.

In some embodiments,

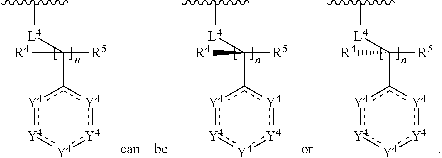

Some embodiments of the compound of Formula (IX) are selected from compounds of Table 9 as shown below, and pharmaceutically acceptable salt thereof.

Formula X

Some embodiments disclosed herein include a compound of Formula (X) as described above or a pharmaceutically acceptable salt thereof.

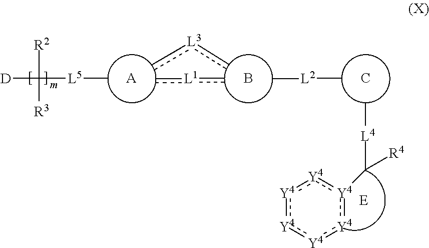

In some embodiments, each of A and B is an acetylene or selected from the group consisting of

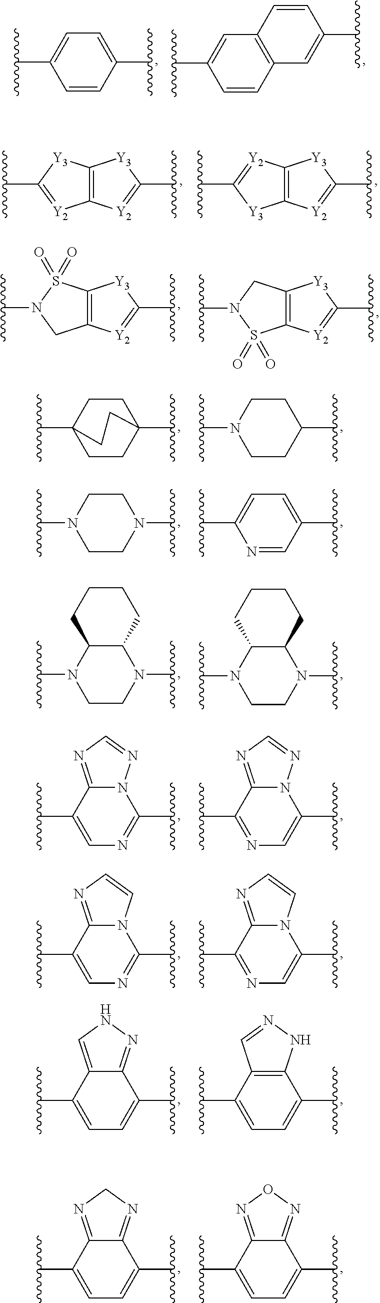

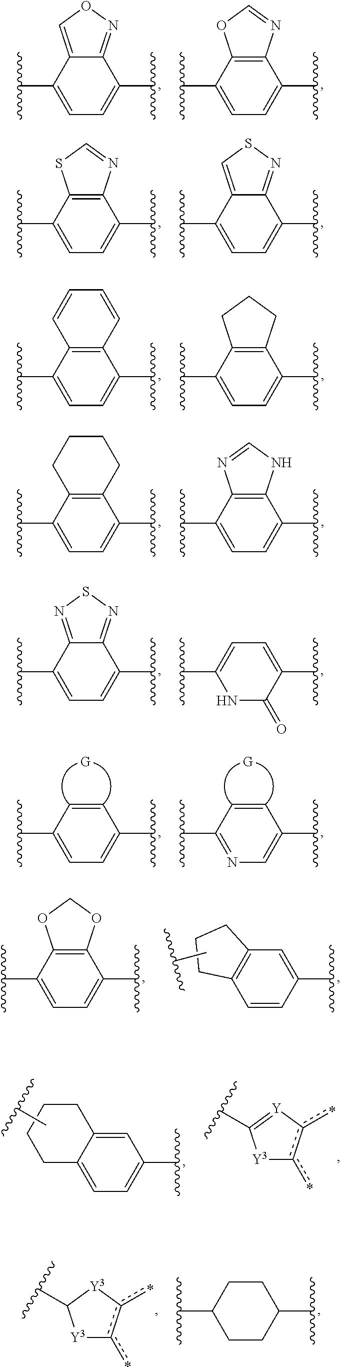

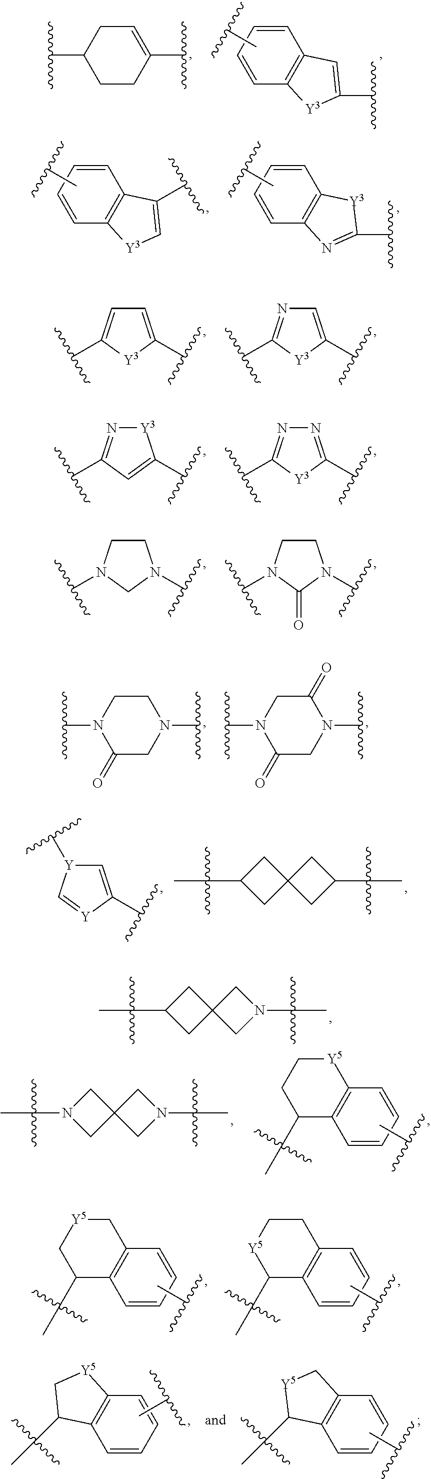

wherein each * is a point of attachment of A or B to L¹ or L³, and wherein the rings in A and B are unsubstituted or substituted with one or more substituents selected from alkyl, haloalkyl, halogen, hydroxy, alkoxy, haloalkoxy, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo;

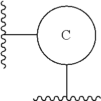

is selected from

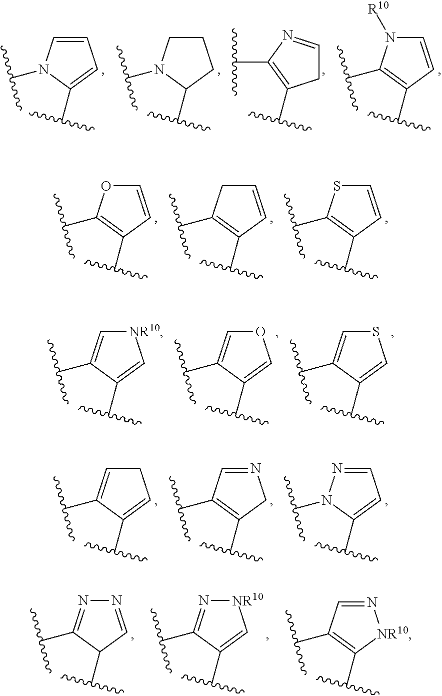

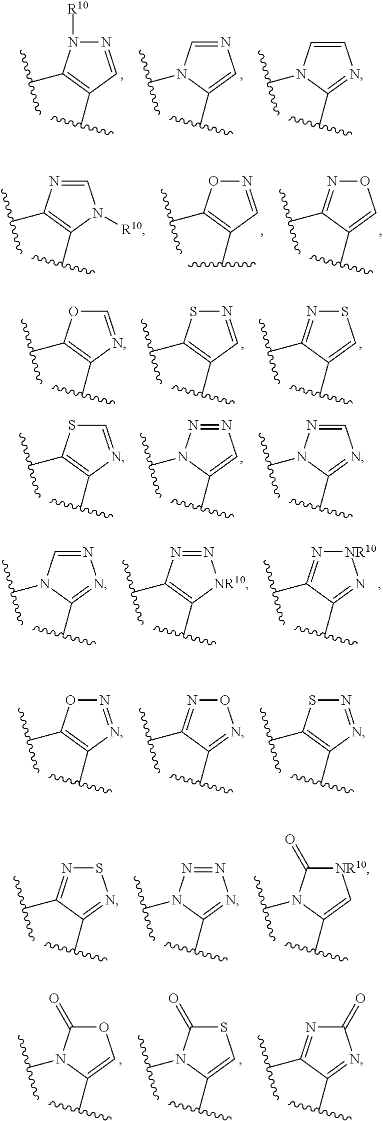

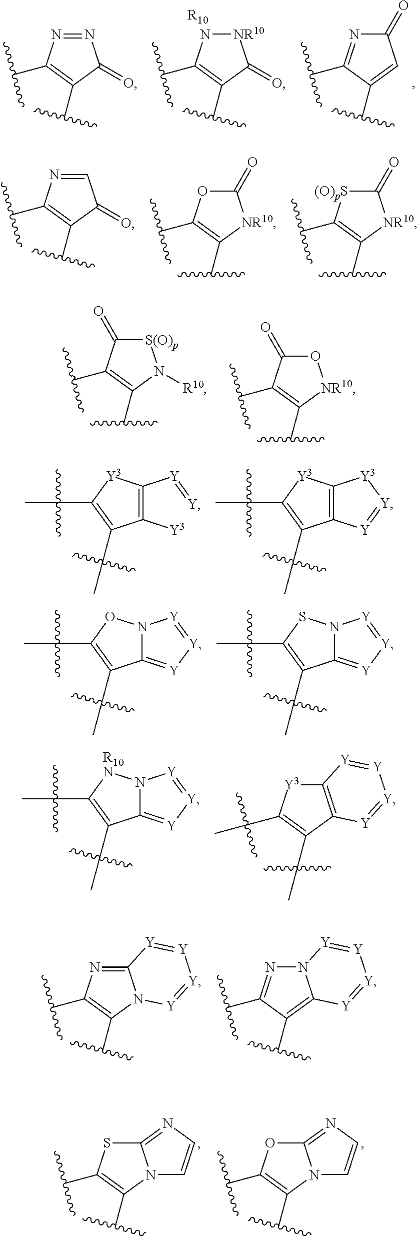

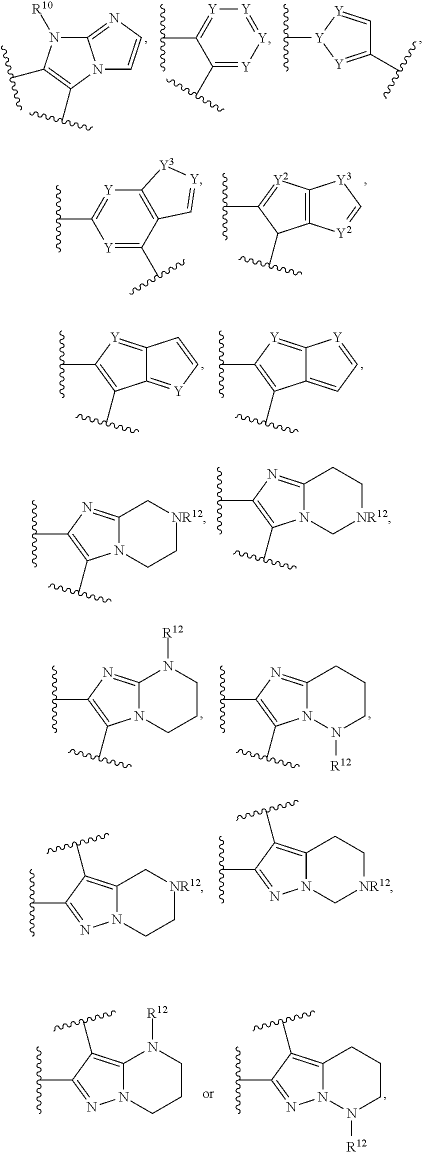

or optionally substituted variants thereof;

Y² is selected from —CH═ or N; Y³ is selected from C(R⁶)₂, NR⁶, O, or S; Y⁵ is selected from NR⁶, O or S; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (X) is also represented by Formula (Xa):

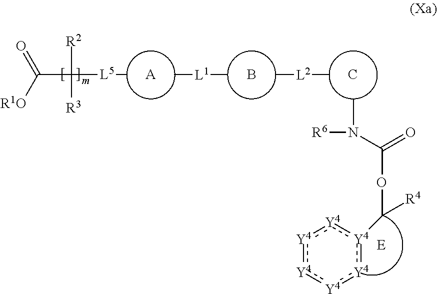

wherein A is phenyl and B is selected from

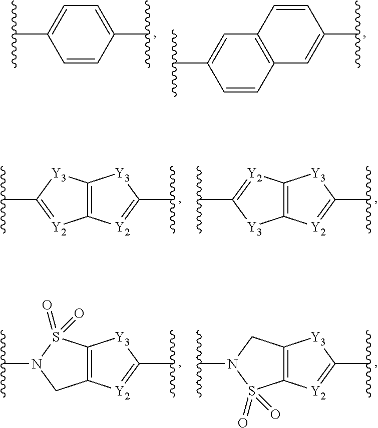

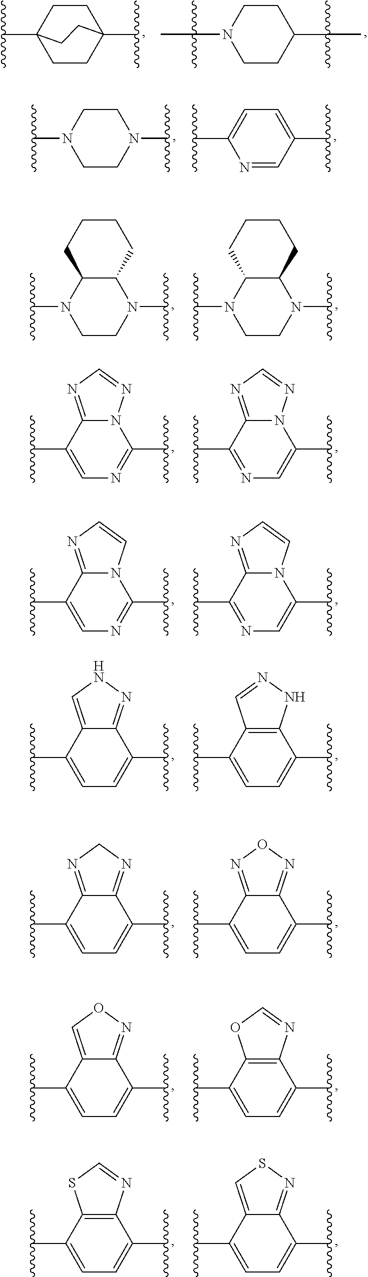

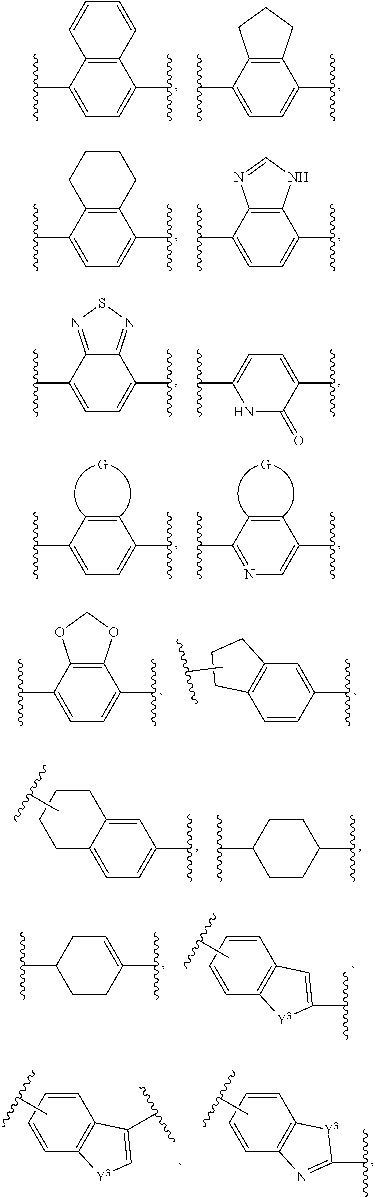

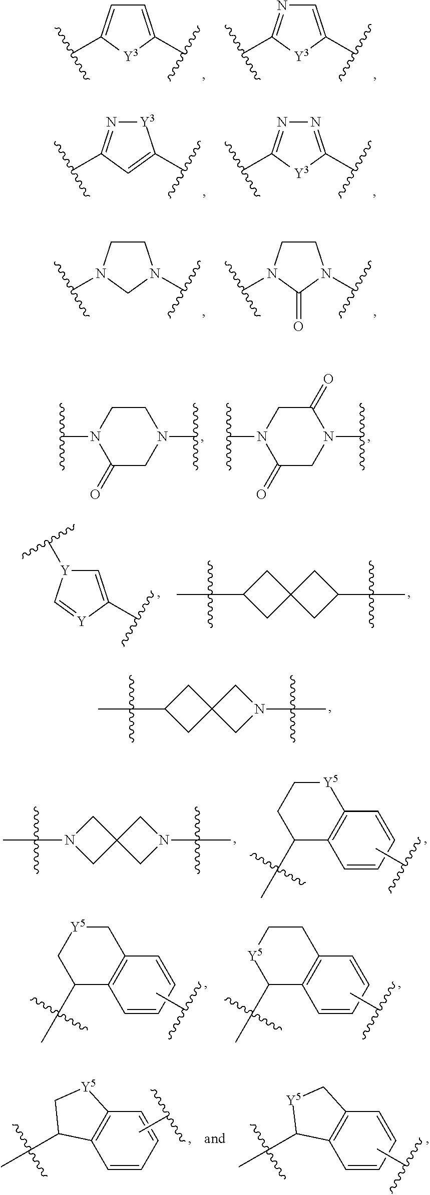

wherein the rings in A and B are unsubstituted or substituted with one or more substituents selected from alkyl, haloalkyl, halogen, hydroxy, alkoxy, haloalkoxy, cyano, or oxo. In some such embodiments, both A and B are phenyl.

In some embodiments, D is selected from

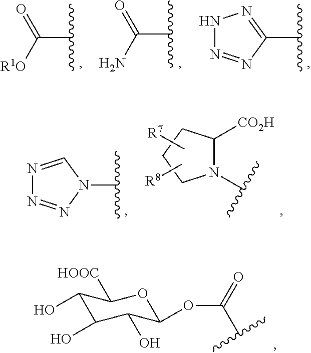

or carboxylic acid isosteres; R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; and each R¹² is independently selected from hydrogen, alkyl, acyl, C-carboxy, C-amido, sulfinyl, sulfonyl, or S-sulfonamido.

Some embodiments described herein of the compound of Formula (X) or (Xa), rings in A or B are unsubstituted.

Some embodiments described herein of the compound of Formula (X) or (Xa), rings in A or B are substituted with one or more substituents selected from alkyl, haloalkyl, halogen, hydroxy, alkoxy, haloalkoxy, cyano, or oxo.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

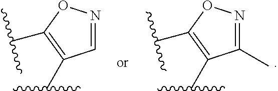

In some embodiments, C is selected from

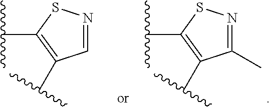

In some embodiments, C is selected from

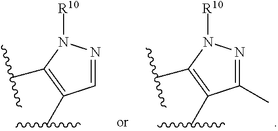

In some embodiments, C is

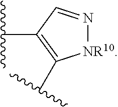

In some embodiments, C is

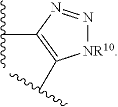

In some embodiments, C is selected from

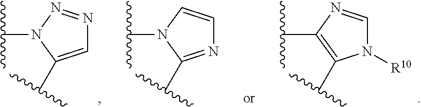

In some such embodiments, R¹⁰ is hydrogen. In some other such embodiments, R¹⁰ is C₁₋₃ alkyl or C₃₋₆ cycloalkyl.

In some embodiments, C is selected from

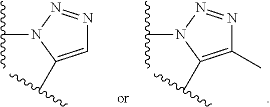

In some embodiments, C is selected from

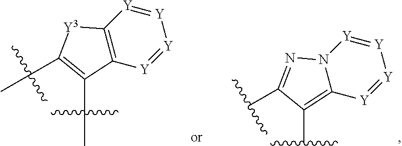

wherein Y³ is selected from O or S. In some such embodiments, Y is a CR⁶. In some other such embodiments, at least one Y is nitrogen.

In some embodiments, C is selected from

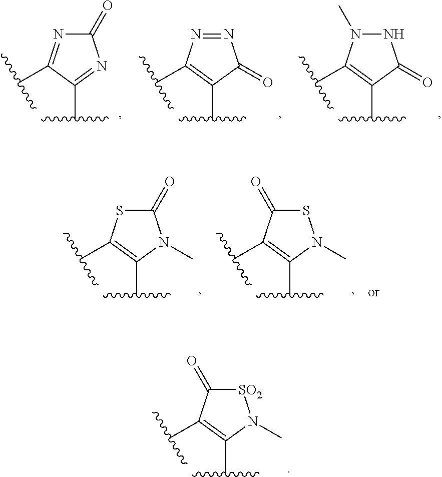

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L¹ is a single bond. In some embodiments, L² is a single bond.

In some embodiments, R⁶ is hydrogen. In some embodiments, R¹ is hydrogen.

In some embodiments, R⁴ is alkyl. In some other embodiments, R⁴ is hydrogen.

Some embodiments of the compound of Formula (X) or (Xa),

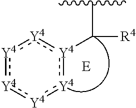

can be selected from

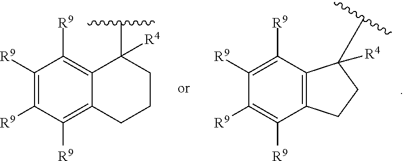

In some such embodiments, each of R⁹ is hydrogen. In some other such embodiments, at least one R⁹ is selected from C₁₋₃ alkyl or halogen.

In some embodiments,

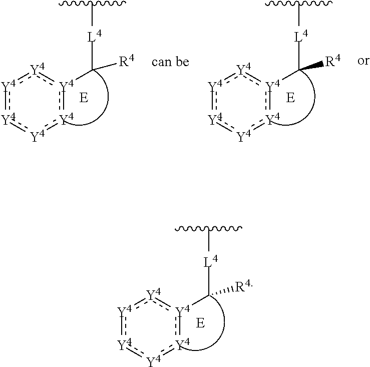

Some embodiments of the compound of Formula (X) are selected from compounds IT057 or IT058, as shown in Table 12.

Formula XI

Some embodiments disclosed herein include a compound of Formula (XI) as described above or a pharmaceutically acceptable salt thereof.

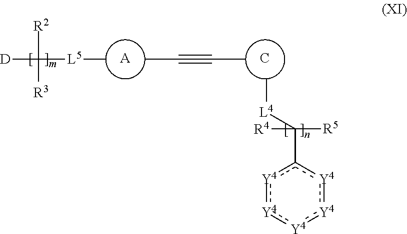

In some embodiments, C is selected from the group consisting of

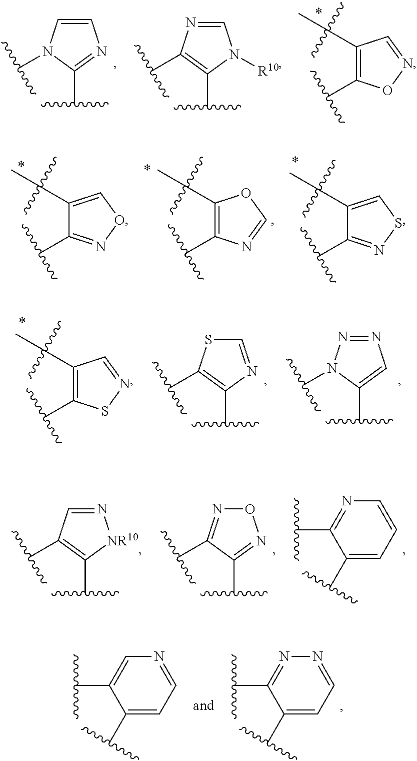

wherein C is unsubstituted or substituted with one or more substituents selected from C₁₋₃ alkyl optionally substituted with halogen or C₁₋₃ alkoxy, C₁₋₆ alkoxy, C₃₋₆ cycloalkyl, halogen, or cyano.

In some embodiments, the compound of Formula (XI) is also represented by Formula (XIa):

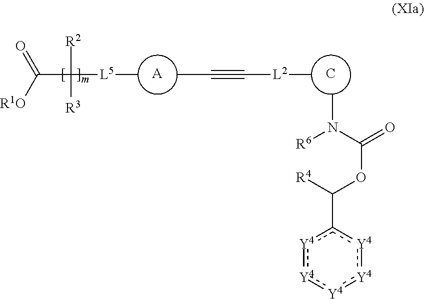

wherein A is selected from the group consisting of

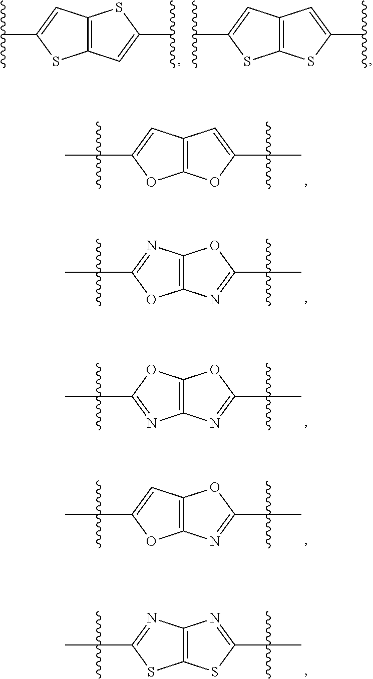

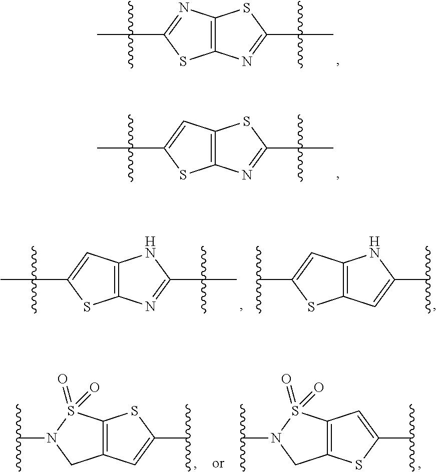 [SMILES: *c1nc2sc(*)nc2s1 ; *c1cc2sc(*)nc2s1 ; *c1nc2sc(*)cc2[nH]1 ; *c1cc2sc(*)cc2[nH]1 ; *c1cc2c(s1)S(=O)(=O)N(*)C2 ; *c1cc2c(s1)CN(*)S2(=O)=O]

wherein the rings in A are unsubstituted or substituted with one or more substituents selected from alkyl, haloalkyl, halogen, hydroxy, alkoxy, haloalkoxy, cyano, or oxo. In some such embodiments, A is selected from

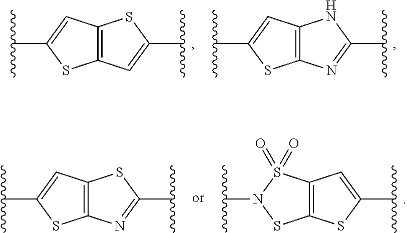

In one embodiment, A is

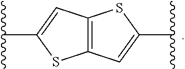 [SMILES: *c1cc2sc(*)cc2s1 ; "."]

In another embodiment, A is

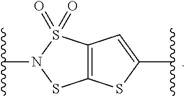 [SMILES: *c1cc2c(s1)SN(*)S2(=O)=O ; "."]

In some embodiments, R¹ is hydrogen or unsubstituted alkyl. In some other embodiments, R¹ is alkyl substituted with one or more substituents selected from the group consisting of alkoxy, C-amido, O-carboxy, and 6 membered heterocyclyl. In still some other embodiments, R¹ is optionally substituted aryl.

In some embodiments, m is 0. In some other embodiments, m is 1. In some other embodiments, m is 2.

In some embodiments, each of R² and R³ is hydrogen. In some other embodiments, one of R² and R³ is hydrogen and the other R² and R³ is aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is R⁶ is hydrogen. In some other embodiments, R is C₁₋₃ alkyl.

In some embodiments, R⁴ is alkyl optionally substituted with halogen. In some other embodiments, R⁴ is hydrogen.

Some embodiments of the compound of Formula (XI) or (XIa),

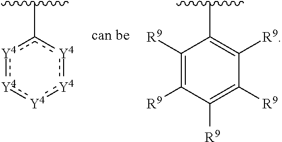

In some embodiments, each of R⁹ is hydrogen. In some other embodiments, at least one R⁹ is selected from C₁₋₃ alkyl or halogen.

In some embodiments,

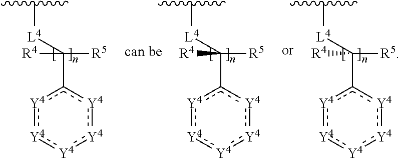

Some embodiments of the compound of Formula (XI) are selected from compounds of Table 10A as shown below, and pharmaceutically acceptable salt thereof.

In one embodiment, the compound of Formula (XI) is IT101 as shown in Table 12.

Some embodiments of the compounds described herein are selected from compounds IT004, IT026-036, IT038-IT045, IT047-IT049, IT052, IT055, IT061, IT064, IT068, IT069, IT072-IT081, IT093, IT094, IT096 and IT102 as shown in Table 12.

Exemplary Compounds

In some embodiments, compounds of Formula (I) are selected from the following compounds as listed in Table 1.

TABLE 1

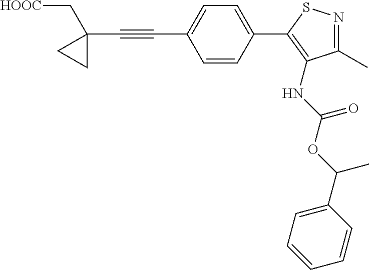 [SMILES: Cc1nsc(-c2ccc(C#CC3(CC(=O)O)CC3)cc2)c1NC(=O)OC(C)c1ccccc1]

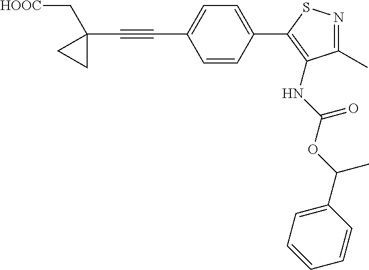 [SMILES: Cc1nsc(-c2ccc(C#CC3(CC(=O)O)CC3)cc2)c1NC(=O)OC(C)c1ccccc1]

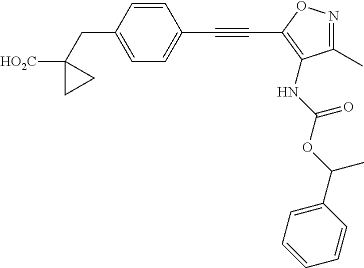 [SMILES: Cc1noc(C#Cc2ccc(CC3(C(=O)O)CC3)cc2)c1NC(=O)OC(C)c1ccccc1]

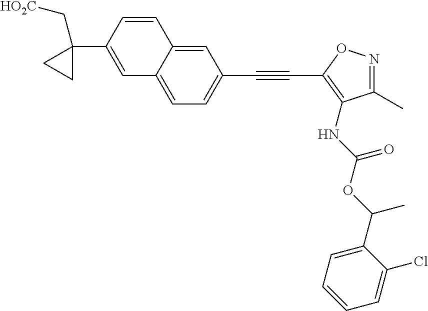 [SMILES: Cc1noc(C#Cc2ccc3cc(C4(CC(=O)O)CC4)ccc3c2)c1NC(=O)OC(C)c1ccccc1Cl]

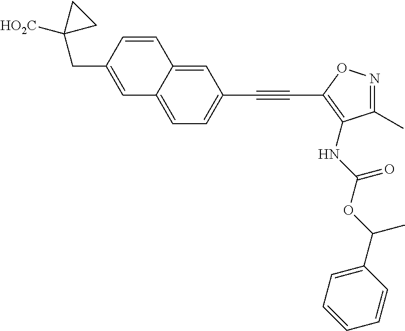 [SMILES: Cc1noc(C#Cc2ccc3cc(CC4(C(=O)O)CC4)ccc3c2)c1NC(=O)OC(C)c1ccccc1]

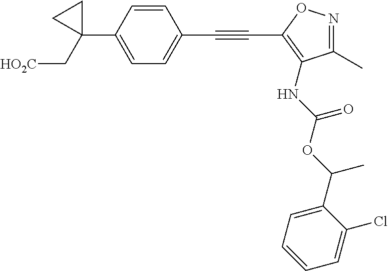 [SMILES: Cc1noc(C#Cc2ccc(C3(CC(=O)O)CC3)cc2)c1NC(=O)OC(C)c1ccccc1Cl]

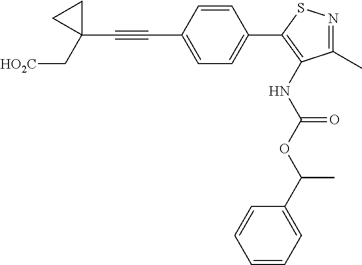 [SMILES: Cc1nsc(-c2ccc(C#CC3(CC(=O)O)CC3)cc2)c1NC(=O)OC(C)c1ccccc1]

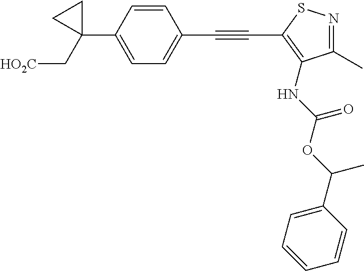 [SMILES: Cc1nsc(C#Cc2ccc(C3(CC(=O)O)CC3)cc2)c1NC(=O)OC(C)c1ccccc1]

In some embodiments, compounds of Formula (II) are selected from the following compounds as listed in Table 2.

TABLE 2

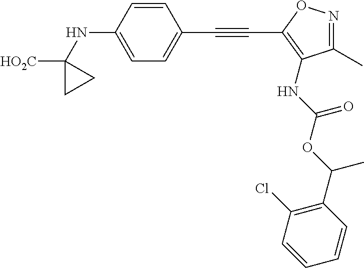 [SMILES: Cc1noc(C#Cc2ccc(NC3(C(=O)O)CC3)cc2)c1NC(=O)OC(C)c1ccccc1Cl]

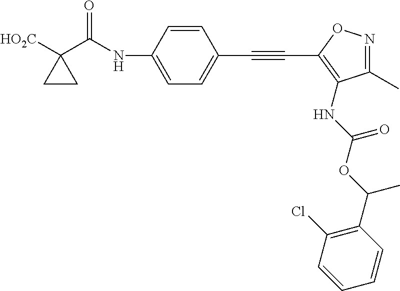 [SMILES: Cc1noc(C#Cc2ccc(NC(=O)C3(C(=O)O)CC3)cc2)c1NC(=O)OC(C)c1ccccc1Cl]

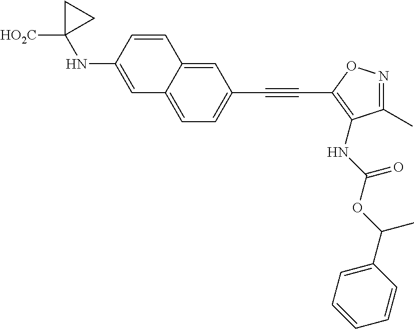 [SMILES: Cc1noc(C#Cc2ccc3cc(NC4(C(=O)O)CC4)ccc3c2)c1NC(=O)OC(C)c1ccccc1]

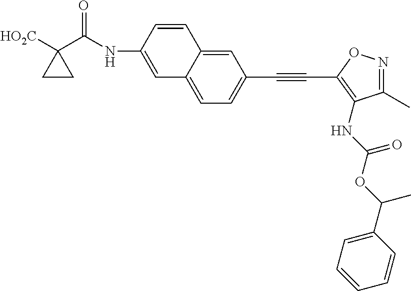 [SMILES: Cc1noc(C#Cc2ccc3cc(NC(=O)C4(C(=O)O)CC4)ccc3c2)c1NC(=O)OC(C)c1ccccc1]

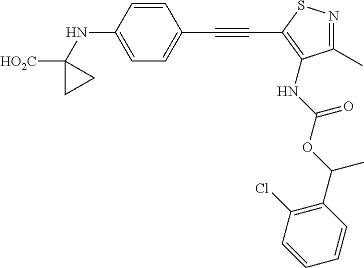 [SMILES: Cc1nsc(C#Cc2ccc(NC3(C(=O)O)CC3)cc2)c1NC(=O)OC(C)c1ccccc1Cl]

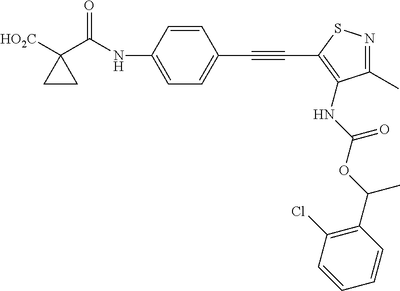 [SMILES: Cc1nsc(C#Cc2ccc(NC(=O)C3(C(=O)O)CC3)cc2)c1NC(=O)OC(C)c1ccccc1Cl]

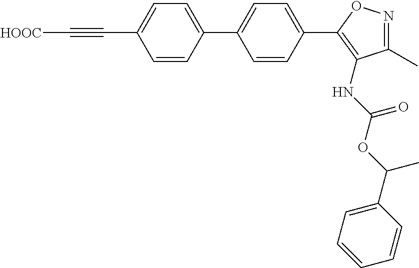 [SMILES: Cc1noc(-c2ccc(-c3ccc(C#CC(=O)O)cc3)cc2)c1NC(=O)OC(C)c1ccccc1]

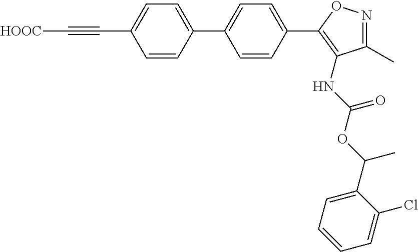 [SMILES: Cc1noc(-c2ccc(-c3ccc(C#CC(=O)O)cc3)cc2)c1NC(=O)OC(C)c1ccccc1Cl]

In some embodiments, compounds of Formula (III) are selected from the following compounds as listed in Table 3.

TABLE 3

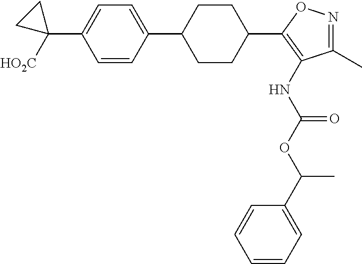 [SMILES: Cc1noc(C2CCC(c3ccc(C4(C(=O)O)CC4)cc3)CC2)c1NC(=O)OC(C)c1ccccc1]

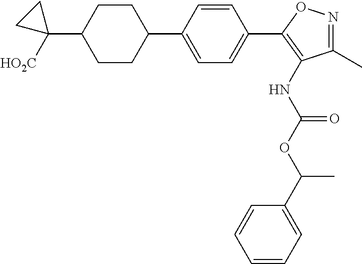 [SMILES: Cc1noc(-c2ccc(C3CCC(C4(C(=O)O)CC4)CC3)cc2)c1NC(=O)OC(C)c1ccccc1]

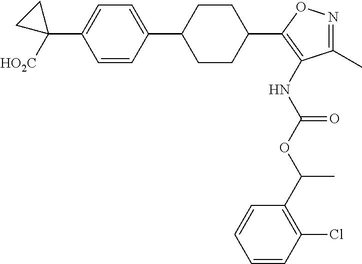 [SMILES: Cc1noc(C2CCC(c3ccc(C4(C(=O)O)CC4)cc3)CC2)c1NC(=O)OC(C)c1ccccc1Cl]

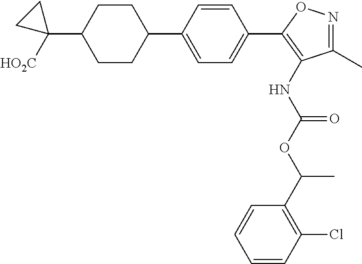 [SMILES: Cc1noc(-c2ccc(C3CCC(C4(C(=O)O)CC4)CC3)cc2)c1NC(=O)OC(C)c1ccccc1Cl]

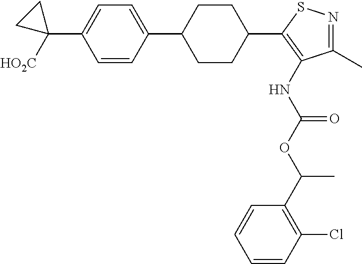 [SMILES: Cc1nsc(C2CCC(c3ccc(C4(C(=O)O)CC4)cc3)CC2)c1NC(=O)OC(C)c1ccccc1Cl]

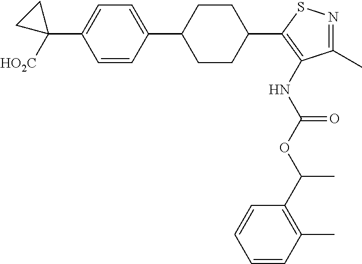 [SMILES: Cc1ccccc1C(C)OC(=O)Nc1c(C)nsc1C1CCC(c2ccc(C3(C(=O)O)CC3)cc2)CC1]

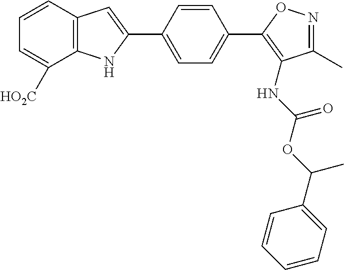 [SMILES: Cc1noc(-c2ccc(-c3cc4cccc(C(=O)O)c4[nH]3)cc2)c1NC(=O)OC(C)c1ccccc1]

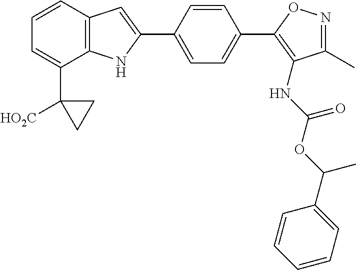 [SMILES: Cc1noc(-c2ccc(-c3cc4cccc(C5(C(=O)O)CC5)c4[nH]3)cc2)c1NC(=O)OC(C)c1ccccc1]

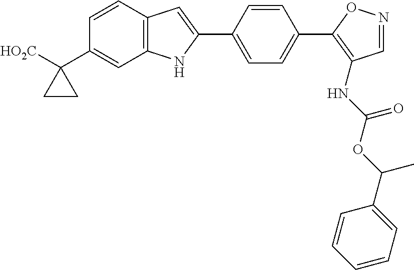 [SMILES: CC(OC(=O)Nc1cnoc1-c1ccc(-c2cc3ccc(C4(C(=O)O)CC4)cc3[nH]2)cc1)c1ccccc1]

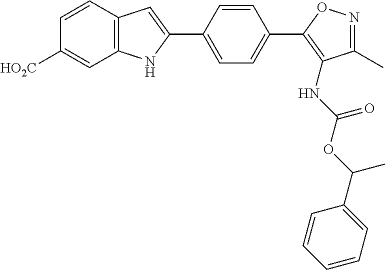 [SMILES: Cc1noc(-c2ccc(-c3cc4ccc(C(=O)O)cc4[nH]3)cc2)c1NC(=O)OC(C)c1ccccc1]

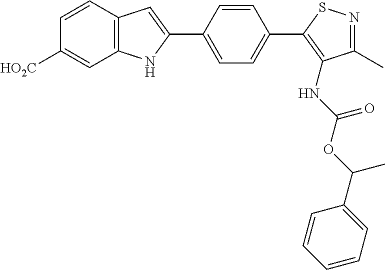 [SMILES: Cc1nsc(-c2ccc(-c3cc4ccc(C(=O)O)cc4[nH]3)cc2)c1NC(=O)OC(C)c1ccccc1]

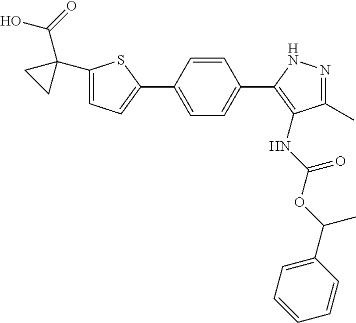 [SMILES: Cc1n[nH]c(-c2ccc(-c3ccc(C4(C(=O)O)CC4)s3)cc2)c1NC(=O)OC(C)c1ccccc1]

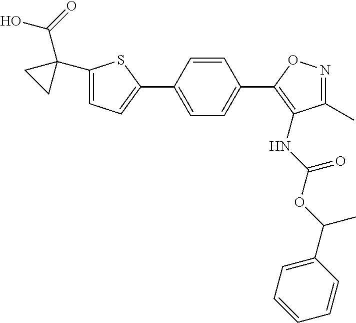 [SMILES: Cc1noc(-c2ccc(-c3ccc(C4(C(=O)O)CC4)s3)cc2)c1NC(=O)OC(C)c1ccccc1]

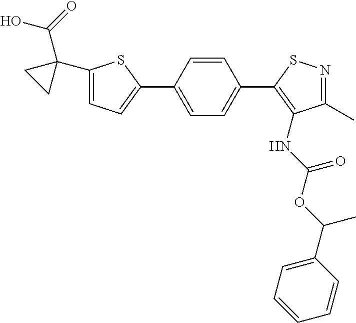 [SMILES: Cc1nsc(-c2ccc(-c3ccc(C4(C(=O)O)CC4)s3)cc2)c1NC(=O)OC(C)c1ccccc1]

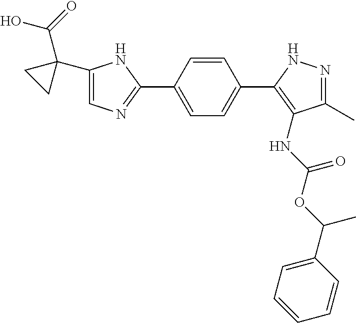 [SMILES: Cc1n[nH]c(-c2ccc(-c3ncc(C4(C(=O)O)CC4)[nH]3)cc2)c1NC(=O)OC(C)c1ccccc1]

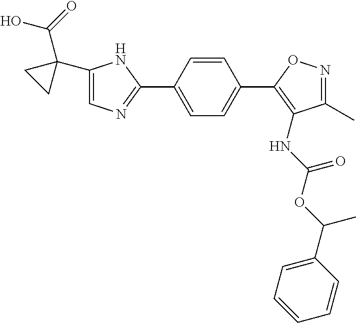 [SMILES: Cc1noc(-c2ccc(-c3ncc(C4(C(=O)O)CC4)[nH]3)cc2)c1NC(=O)OC(C)c1ccccc1]

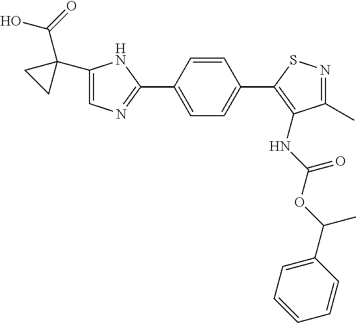 [SMILES: Cc1nsc(-c2ccc(-c3ncc(C4(C(=O)O)CC4)[nH]3)cc2)c1NC(=O)OC(C)c1ccccc1]

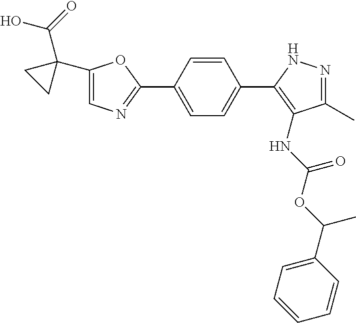 [SMILES: Cc1n[nH]c(-c2ccc(-c3ncc(C4(C(=O)O)CC4)o3)cc2)c1NC(=O)OC(C)c1ccccc1]

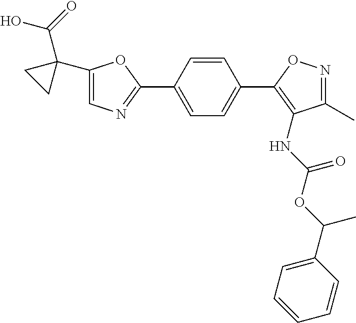 [SMILES: Cc1noc(-c2ccc(-c3ncc(C4(C(=O)O)CC4)o3)cc2)c1NC(=O)OC(C)c1ccccc1]

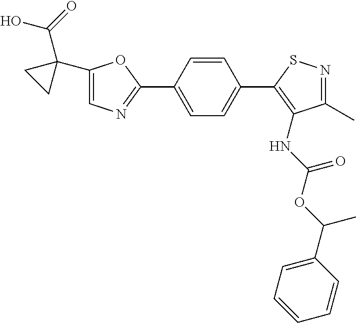 [SMILES: Cc1nsc(-c2ccc(-c3ncc(C4(C(=O)O)CC4)o3)cc2)c1NC(=O)OC(C)c1ccccc1]

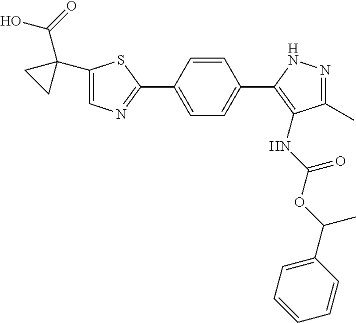 [SMILES: Cc1n[nH]c(-c2ccc(-c3ncc(C4(C(=O)O)CC4)s3)cc2)c1NC(=O)OC(C)c1ccccc1]

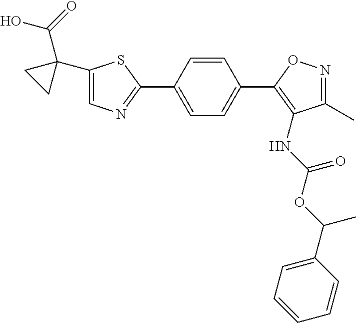 [SMILES: Cc1noc(-c2ccc(-c3ncc(C4(C(=O)O)CC4)s3)cc2)c1NC(=O)OC(C)c1ccccc1]

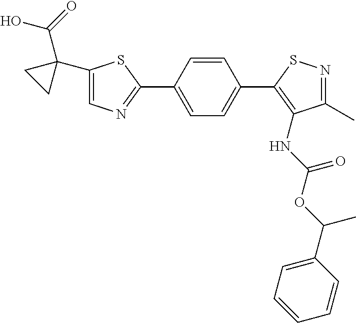 [SMILES: Cc1nsc(-c2ccc(-c3ncc(C4(C(=O)O)CC4)s3)cc2)c1NC(=O)OC(C)c1ccccc1]

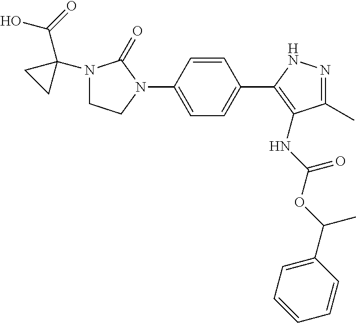 [SMILES: Cc1n[nH]c(-c2ccc(N3CCN(C4(C(=O)O)CC4)C3=O)cc2)c1NC(=O)OC(C)c1ccccc1]

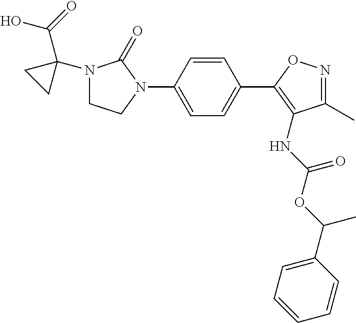 [SMILES: Cc1noc(-c2ccc(N3CCN(C4(C(=O)O)CC4)C3=O)cc2)c1NC(=O)OC(C)c1ccccc1]

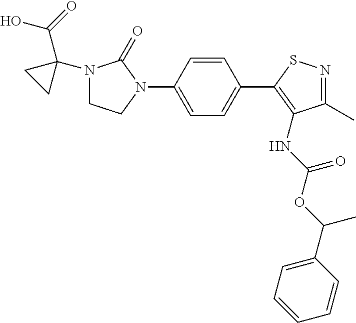 [SMILES: Cc1nsc(-c2ccc(N3CCN(C4(C(=O)O)CC4)C3=O)cc2)c1NC(=O)OC(C)c1ccccc1]

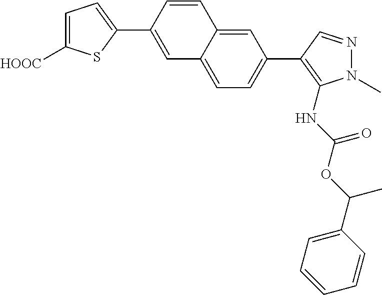 [SMILES: CC(OC(=O)Nc1c(-c2ccc3cc(-c4ccc(C(=O)O)s4)ccc3c2)cnn1C)c1ccccc1]

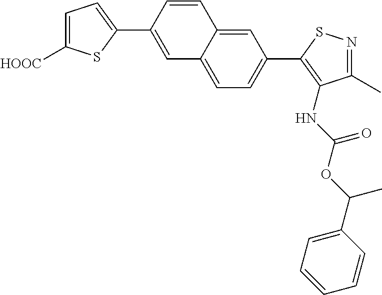 [SMILES: Cc1nsc(-c2ccc3cc(-c4ccc(C(=O)O)s4)ccc3c2)c1NC(=O)OC(C)c1ccccc1]

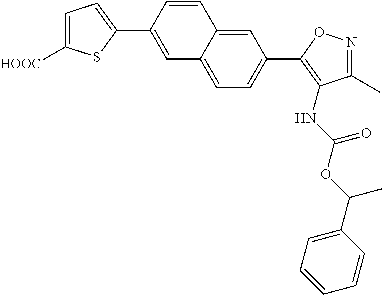 [SMILES: Cc1noc(-c2ccc3cc(-c4ccc(C(=O)O)s4)ccc3c2)c1NC(=O)OC(C)c1ccccc1]

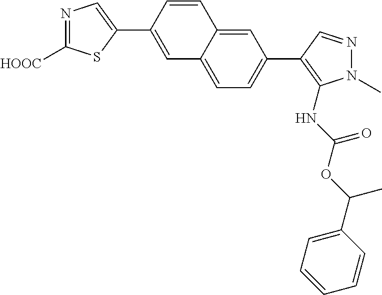 [SMILES: CC(OC(=O)Nc1c(-c2ccc3cc(-c4cnc(C(=O)O)s4)ccc3c2)cnn1C)c1ccccc1]

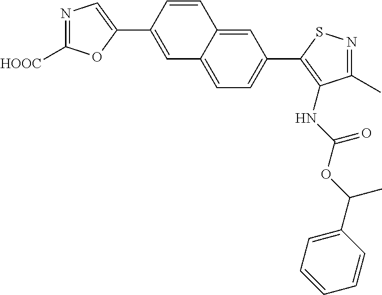 [SMILES: Cc1nsc(-c2ccc3cc(-c4cnc(C(=O)O)o4)ccc3c2)c1NC(=O)OC(C)c1ccccc1]

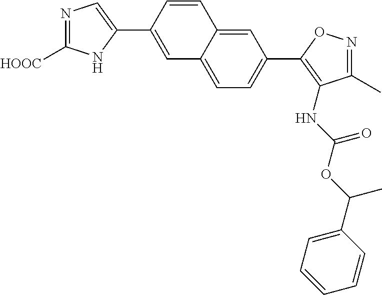 [SMILES: Cc1noc(-c2ccc3cc(-c4cnc(C(=O)O)[nH]4)ccc3c2)c1NC(=O)OC(C)c1ccccc1]

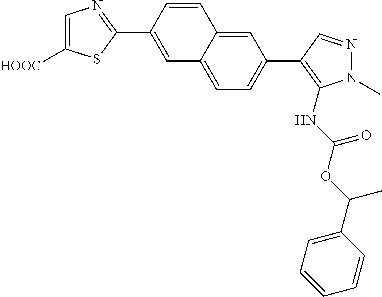 [SMILES: CC(OC(=O)Nc1c(-c2ccc3cc(-c4ncc(C(=O)O)s4)ccc3c2)cnn1C)c1ccccc1]

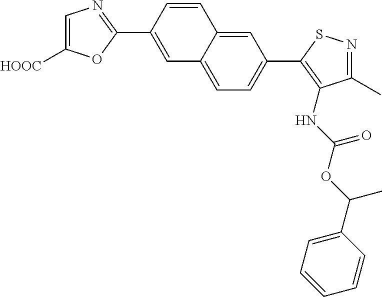 [SMILES: Cc1nsc(-c2ccc3cc(-c4ncc(C(=O)O)o4)ccc3c2)c1NC(=O)OC(C)c1ccccc1]

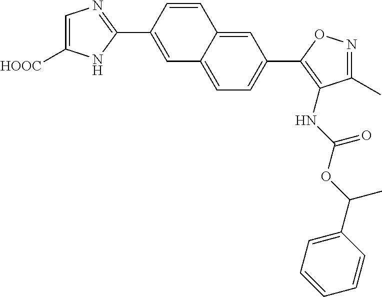 [SMILES: Cc1noc(-c2ccc3cc(-c4ncc(C(=O)O)[nH]4)ccc3c2)c1NC(=O)OC(C)c1ccccc1]

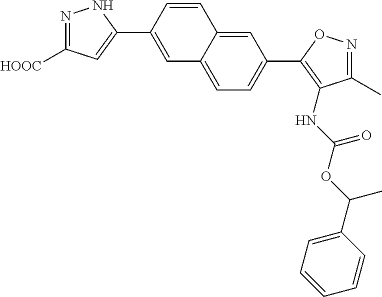 [SMILES: Cc1noc(-c2ccc3cc(-c4cc(C(=O)O)n[nH]4)ccc3c2)c1NC(=O)OC(C)c1ccccc1]

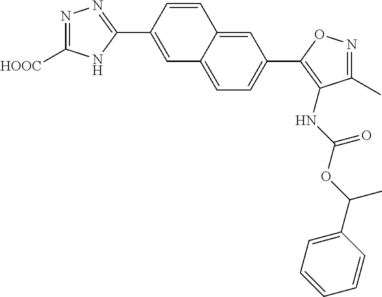 [SMILES: Cc1noc(-c2ccc3cc(-c4nnc(C(=O)O)[nH]4)ccc3c2)c1NC(=O)OC(C)c1ccccc1]

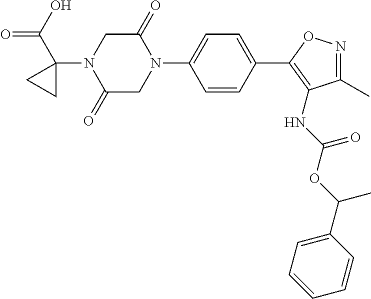 [SMILES: Cc1noc(-c2ccc(N3CC(=O)N(C4(C(=O)O)CC4)CC3=O)cc2)c1NC(=O)OC(C)c1ccccc1]

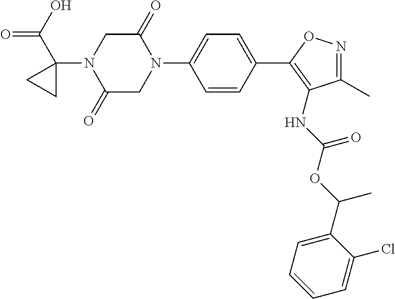 [SMILES: Cc1noc(-c2ccc(N3CC(=O)N(C4(C(=O)O)CC4)CC3=O)cc2)c1NC(=O)OC(C)c1ccccc1Cl]

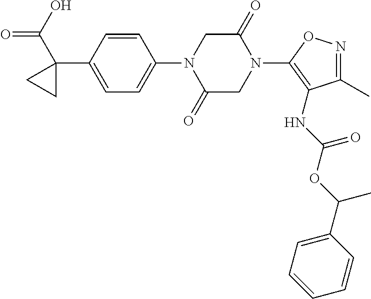 [SMILES: Cc1noc(N2CC(=O)N(c3ccc(C4(C(=O)O)CC4)cc3)CC2=O)c1NC(=O)OC(C)c1ccccc1]

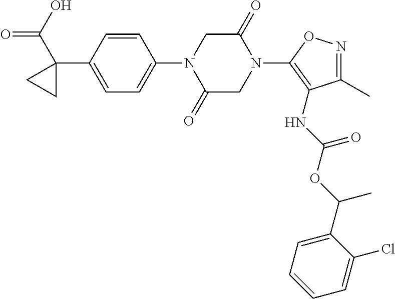 [SMILES: Cc1noc(N2CC(=O)N(c3ccc(C4(C(=O)O)CC4)cc3)CC2=O)c1NC(=O)OC(C)c1ccccc1Cl]

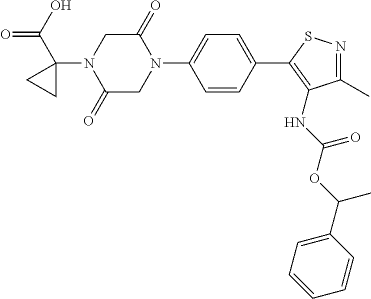 [SMILES: Cc1nsc(-c2ccc(N3CC(=O)N(C4(C(=O)O)CC4)CC3=O)cc2)c1NC(=O)OC(C)c1ccccc1]

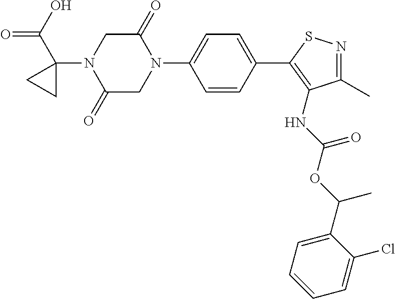 [SMILES: Cc1nsc(-c2ccc(N3CC(=O)N(C4(C(=O)O)CC4)CC3=O)cc2)c1NC(=O)OC(C)c1ccccc1Cl]

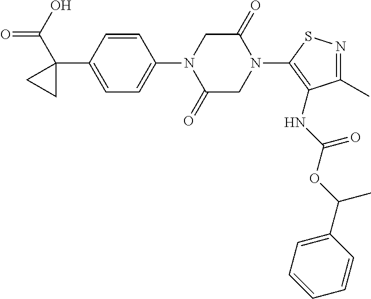 [SMILES: Cc1nsc(N2CC(=O)N(c3ccc(C4(C(=O)O)CC4)cc3)CC2=O)c1NC(=O)OC(C)c1ccccc1]

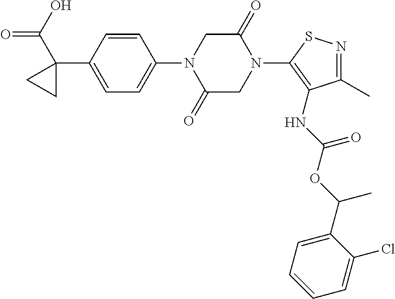 [SMILES: Cc1nsc(N2CC(=O)N(c3ccc(C4(C(=O)O)CC4)cc3)CC2=O)c1NC(=O)OC(C)c1ccccc1Cl]

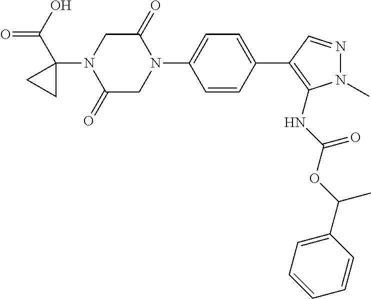 [SMILES: CC(OC(=O)Nc1c(-c2ccc(N3CC(=O)N(C4(C(=O)O)CC4)CC3=O)cc2)cnn1C)c1ccccc1]

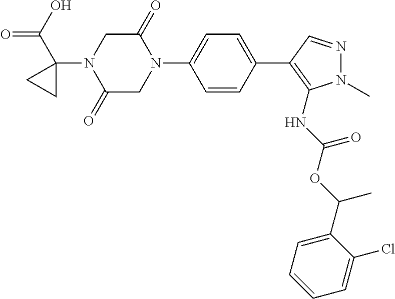 [SMILES: CC(OC(=O)Nc1c(-c2ccc(N3CC(=O)N(C4(C(=O)O)CC4)CC3=O)cc2)cnn1C)c1ccccc1Cl]

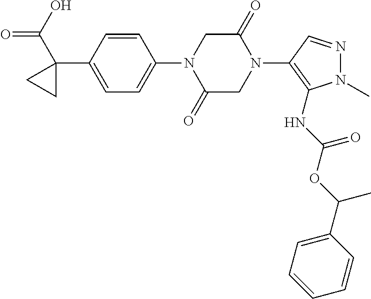 [SMILES: CC(OC(=O)Nc1c(N2CC(=O)N(c3ccc(C4(C(=O)O)CC4)cc3)CC2=O)cnn1C)c1ccccc1]

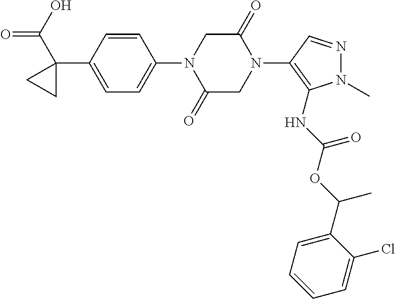 [SMILES: CC(OC(=O)Nc1c(N2CC(=O)N(c3ccc(C4(C(=O)O)CC4)cc3)CC2=O)cnn1C)c1ccccc1Cl]

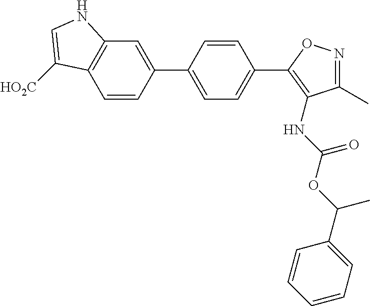 [SMILES: Cc1noc(-c2ccc(-c3ccc4c(C(=O)O)c[nH]c4c3)cc2)c1NC(=O)OC(C)c1ccccc1]

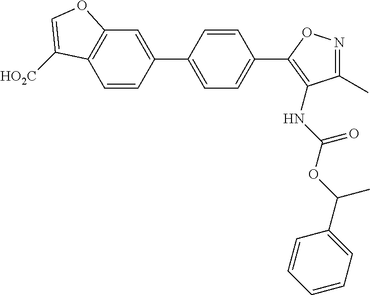 [SMILES: Cc1noc(-c2ccc(-c3ccc4c(C(=O)O)coc4c3)cc2)c1NC(=O)OC(C)c1ccccc1]

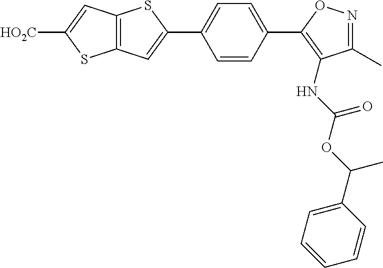 [SMILES: Cc1noc(-c2ccc(-c3cc4sc(C(=O)O)cc4s3)cc2)c1NC(=O)OC(C)c1ccccc1]

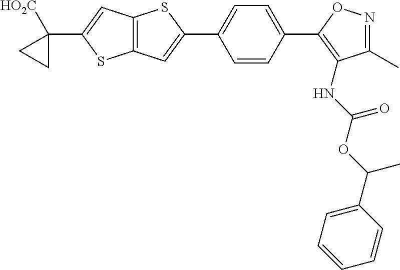 [SMILES: Cc1noc(-c2ccc(-c3cc4sc(C5(C(=O)O)CC5)cc4s3)cc2)c1NC(=O)OC(C)c1ccccc1]

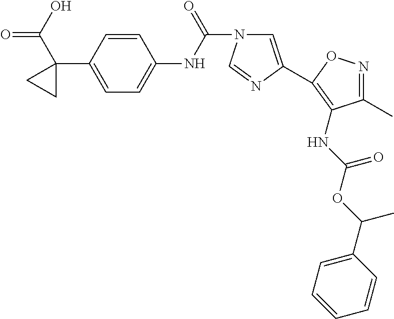 [SMILES: Cc1noc(-c2cn(C(=O)Nc3ccc(C4(C(=O)O)CC4)cc3)cn2)c1NC(=O)OC(C)c1ccccc1]

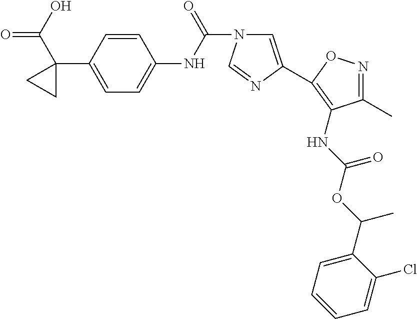 [SMILES: Cc1noc(-c2cn(C(=O)Nc3ccc(C4(C(=O)O)CC4)cc3)cn2)c1NC(=O)OC(C)c1ccccc1Cl]

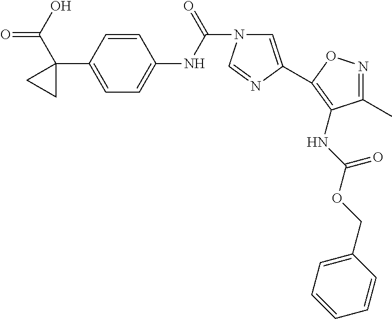 [SMILES: Cc1noc(-c2cn(C(=O)Nc3ccc(C4(C(=O)O)CC4)cc3)cn2)c1NC(=O)OCc1ccccc1]

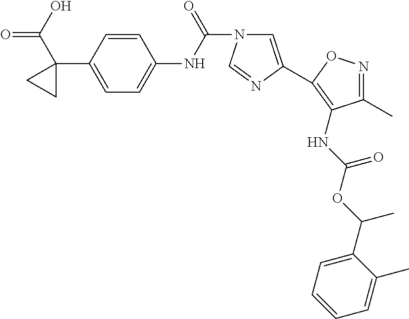 [SMILES: Cc1ccccc1C(C)OC(=O)Nc1c(C)noc1-c1cn(C(=O)Nc2ccc(C3(C(=O)O)CC3)cc2)cn1]

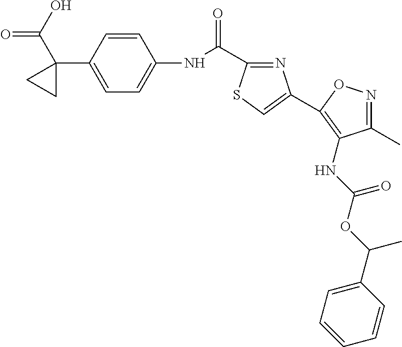 [SMILES: Cc1noc(-c2csc(C(=O)Nc3ccc(C4(C(=O)O)CC4)cc3)n2)c1NC(=O)OC(C)c1ccccc1]

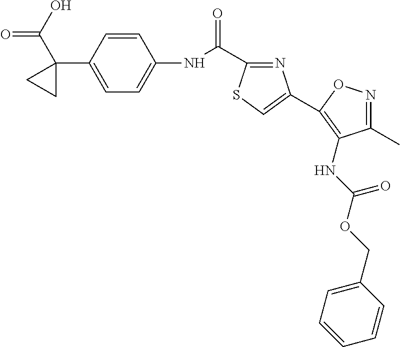 [SMILES: Cc1noc(-c2csc(C(=O)Nc3ccc(C4(C(=O)O)CC4)cc3)n2)c1NC(=O)OCc1ccccc1]

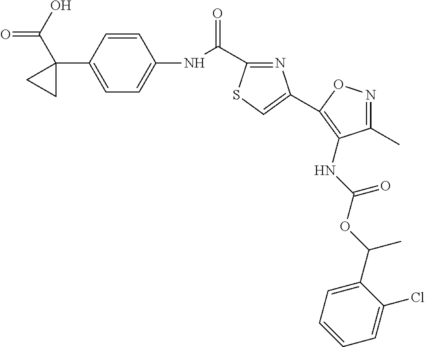 [SMILES: Cc1noc(-c2csc(C(=O)Nc3ccc(C4(C(=O)O)CC4)cc3)n2)c1NC(=O)OC(C)c1ccccc1Cl]

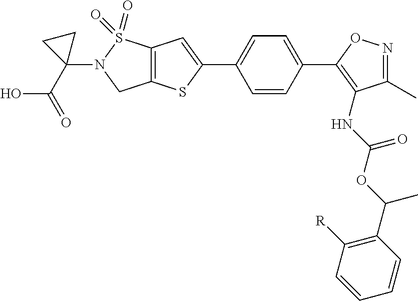

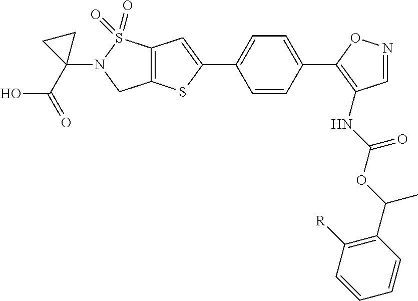

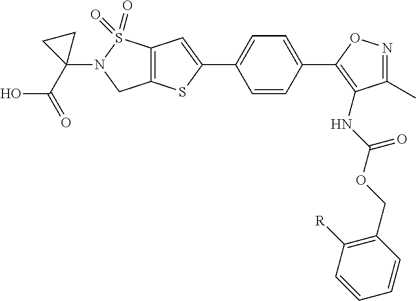

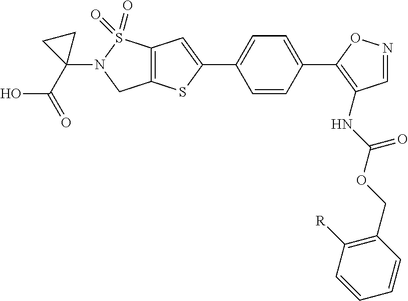

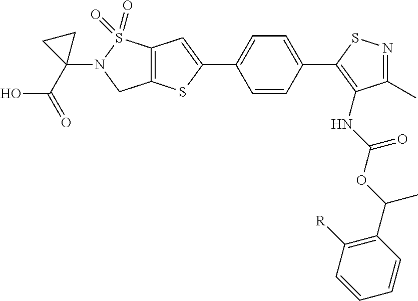

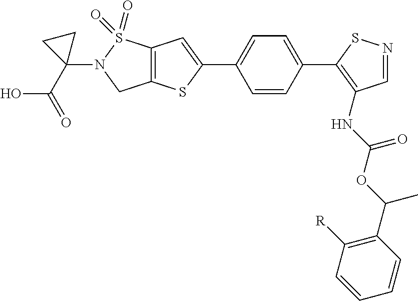

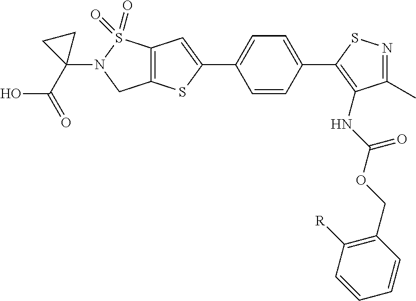

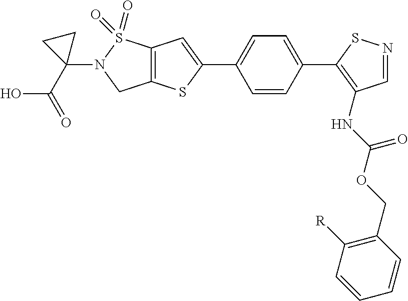

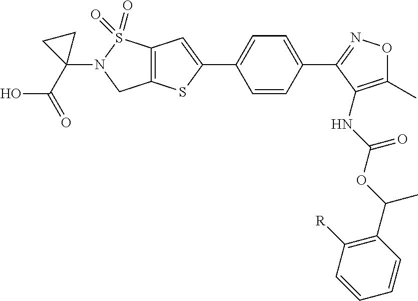

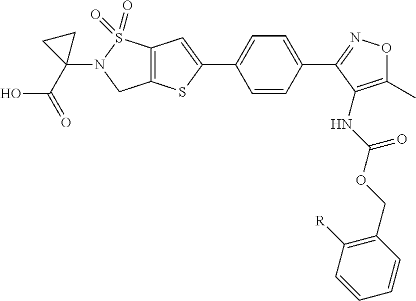

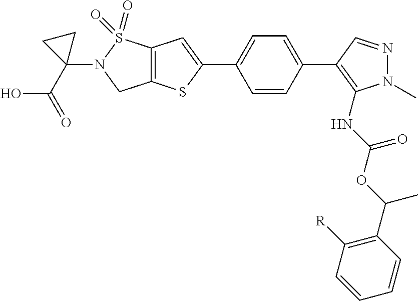

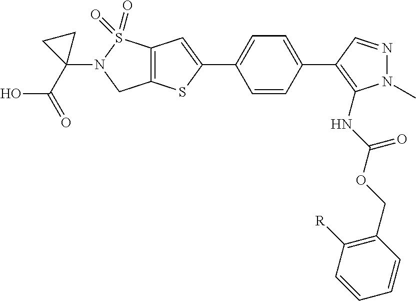

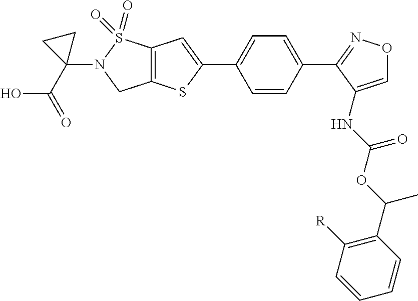

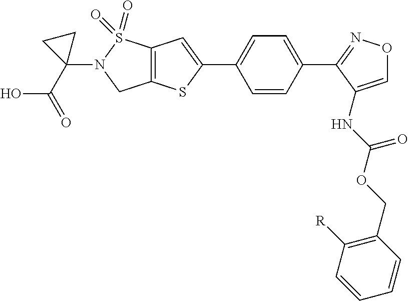

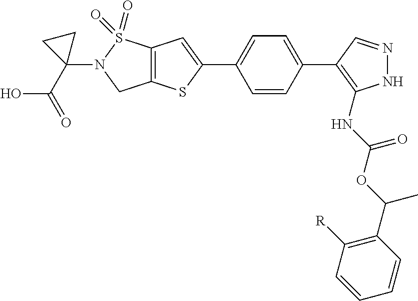

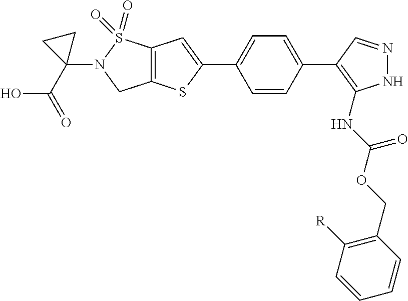

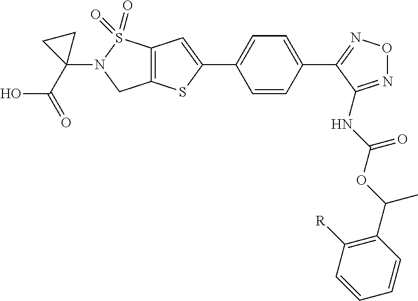

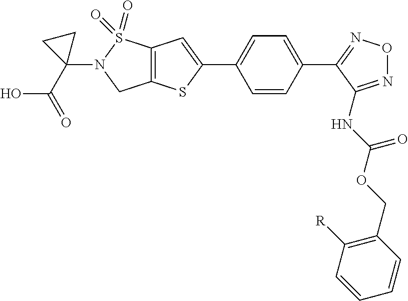

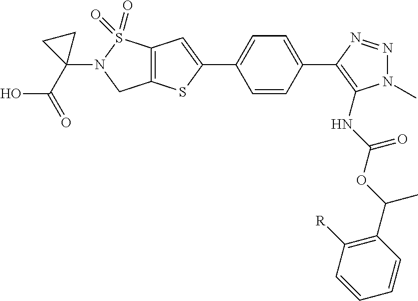

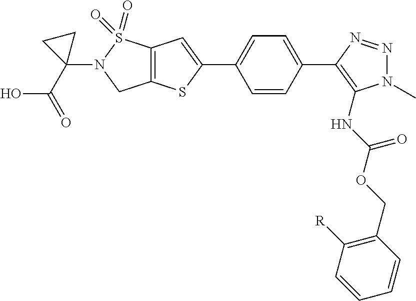

R = H, Me or Cl

In some embodiments, compounds of Formula (IV) are selected from the following compounds as listed in Table 4.

TABLE 4

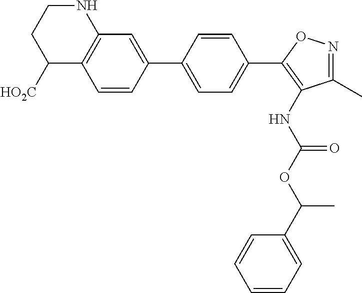 [SMILES: Cc1noc(-c2ccc(-c3ccc4c(c3)NCCC4C(=O)O)cc2)c1NC(=O)OC(C)c1ccccc1]

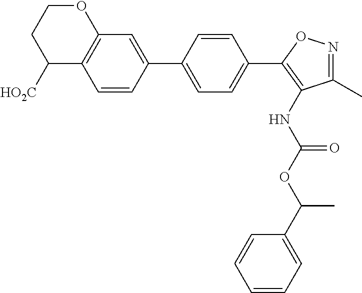 [SMILES: Cc1noc(-c2ccc(-c3ccc4c(c3)OCCC4C(=O)O)cc2)c1NC(=O)OC(C)c1ccccc1]

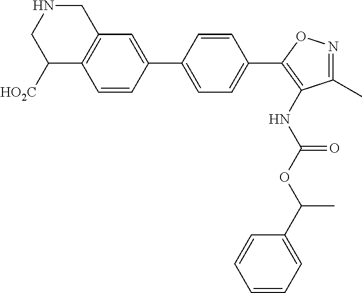 [SMILES: Cc1noc(-c2ccc(-c3ccc4c(c3)CNCC4C(=O)O)cc2)c1NC(=O)OC(C)c1ccccc1]

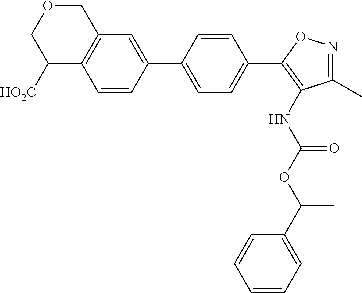 [SMILES: Cc1noc(-c2ccc(-c3ccc4c(c3)COCC4C(=O)O)cc2)c1NC(=O)OC(C)c1ccccc1]

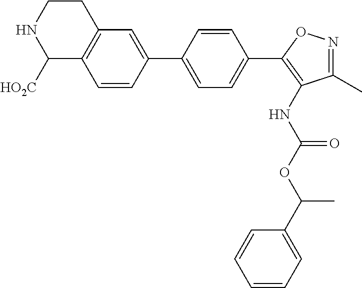 [SMILES: Cc1noc(-c2ccc(-c3ccc4c(c3)CCNC4C(=O)O)cc2)c1NC(=O)OC(C)c1ccccc1]

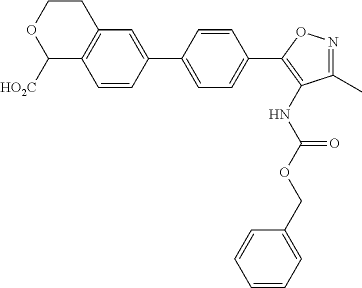 [SMILES: Cc1noc(-c2ccc(-c3ccc4c(c3)CCOC4C(=O)O)cc2)c1NC(=O)OCc1ccccc1]

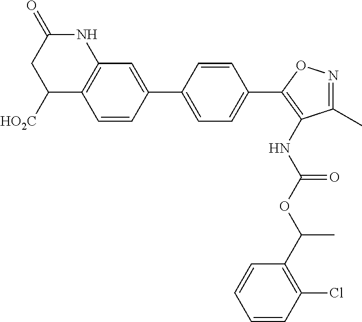 [SMILES: Cc1noc(-c2ccc(-c3ccc4c(c3)NC(=O)CC4C(=O)O)cc2)c1NC(=O)OC(C)c1ccccc1Cl]

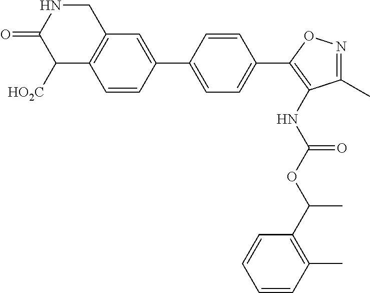 [SMILES: Cc1ccccc1C(C)OC(=O)Nc1c(C)noc1-c1ccc(-c2ccc3c(c2)CNC(=O)C3C(=O)O)cc1]

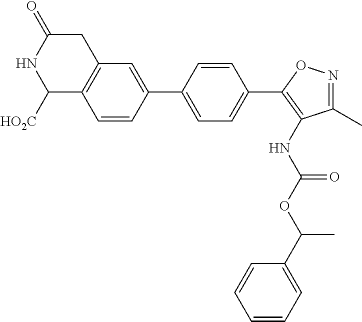 [SMILES: Cc1noc(-c2ccc(-c3ccc4c(c3)CC(=O)NC4C(=O)O)cc2)c1NC(=O)OC(C)c1ccccc1]

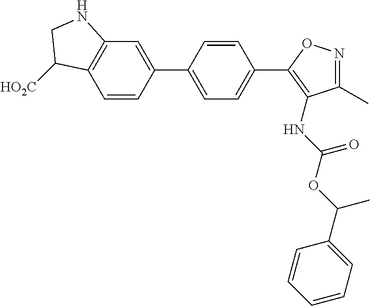 [SMILES: Cc1noc(-c2ccc(-c3ccc4c(c3)NCC4C(=O)O)cc2)c1NC(=O)OC(C)c1ccccc1]

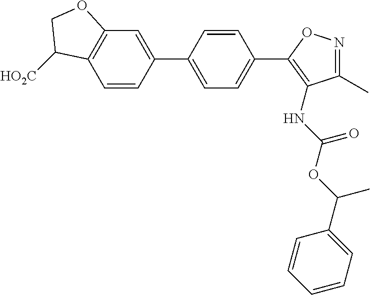 [SMILES: Cc1noc(-c2ccc(-c3ccc4c(c3)OCC4C(=O)O)cc2)c1NC(=O)OC(C)c1ccccc1]

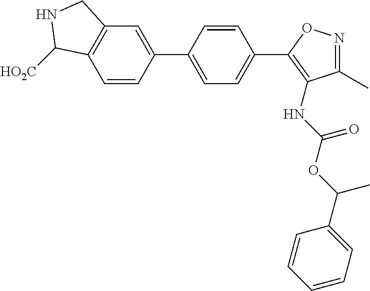 [SMILES: Cc1noc(-c2ccc(-c3ccc4c(c3)CNC4C(=O)O)cc2)c1NC(=O)OC(C)c1ccccc1]

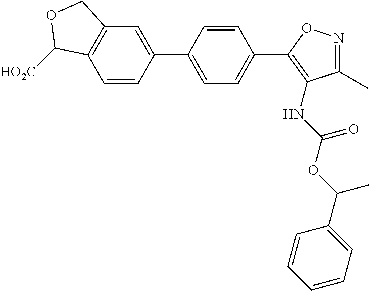 [SMILES: Cc1noc(-c2ccc(-c3ccc4c(c3)COC4C(=O)O)cc2)c1NC(=O)OC(C)c1ccccc1]

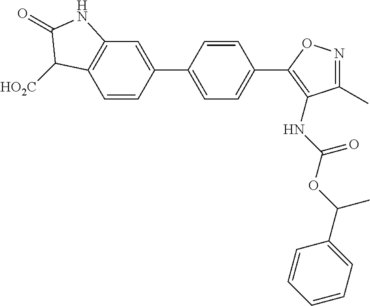 [SMILES: Cc1noc(-c2ccc(-c3ccc4c(c3)NC(=O)C4C(=O)O)cc2)c1NC(=O)OC(C)c1ccccc1]

In some embodiments, compounds of Formula (V) are selected from the following compounds as listed in Table 5.

TABLE 5

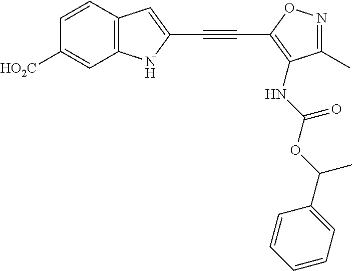 [SMILES: Cc1noc(C#Cc2cc3ccc(C(=O)O)cc3[nH]2)c1NC(=O)OC(C)c1ccccc1]

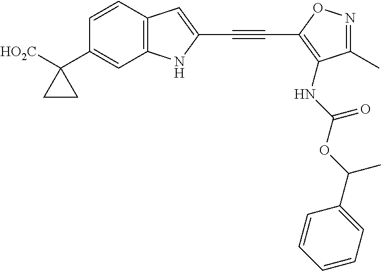 [SMILES: Cc1noc(C#Cc2cc3ccc(C4(C(=O)O)CC4)cc3[nH]2)c1NC(=O)OC(C)c1ccccc1]

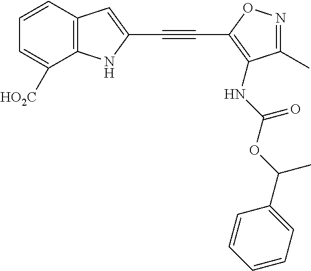 [SMILES: Cc1noc(C#Cc2cc3cccc(C(=O)O)c3[nH]2)c1NC(=O)OC(C)c1ccccc1]

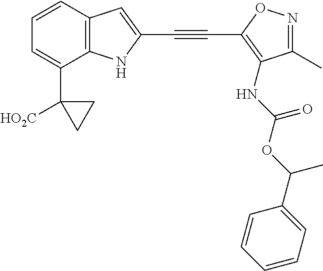 [SMILES: Cc1noc(C#Cc2cc3cccc(C4(C(=O)O)CC4)c3[nH]2)c1NC(=O)OC(C)c1ccccc1]

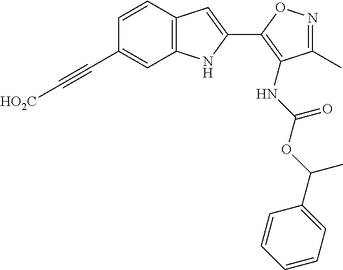 [SMILES: Cc1noc(-c2cc3ccc(C#CC(=O)O)cc3[nH]2)c1NC(=O)OC(C)c1ccccc1]

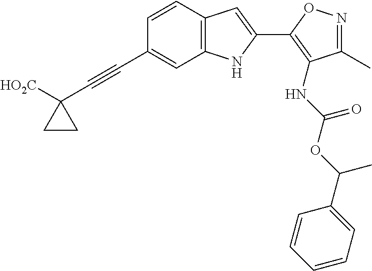 [SMILES: Cc1noc(-c2cc3ccc(C#CC4(C(=O)O)CC4)cc3[nH]2)c1NC(=O)OC(C)c1ccccc1]

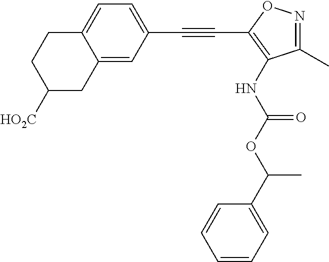 [SMILES: Cc1noc(C#Cc2ccc3c(c2)CC(C(=O)O)CC3)c1NC(=O)OC(C)c1ccccc1]

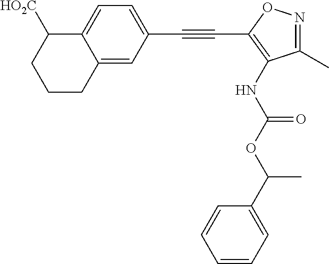 [SMILES: Cc1noc(C#Cc2ccc3c(c2)CCCC3C(=O)O)c1NC(=O)OC(C)c1ccccc1]

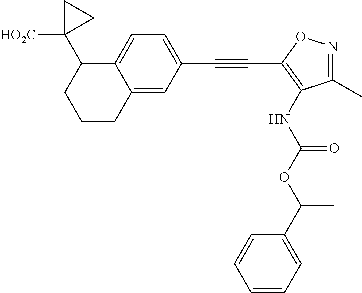 [SMILES: Cc1noc(C#Cc2ccc3c(c2)CCCC3C2(C(=O)O)CC2)c1NC(=O)OC(C)c1ccccc1]

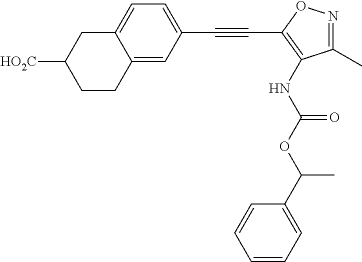 [SMILES: Cc1noc(C#Cc2ccc3c(c2)CCC(C(=O)O)C3)c1NC(=O)OC(C)c1ccccc1]

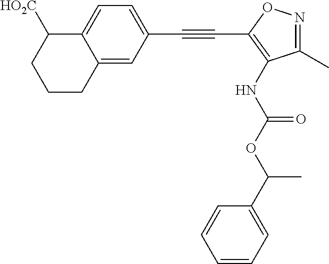 [SMILES: Cc1noc(C#Cc2ccc3c(c2)CCCC3C(=O)O)c1NC(=O)OC(C)c1ccccc1]

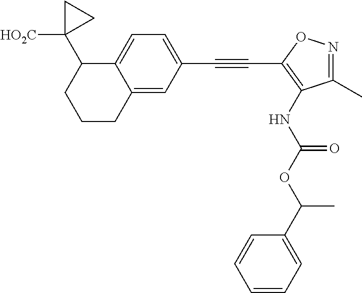 [SMILES: Cc1noc(C#Cc2ccc3c(c2)CCCC3C2(C(=O)O)CC2)c1NC(=O)OC(C)c1ccccc1]

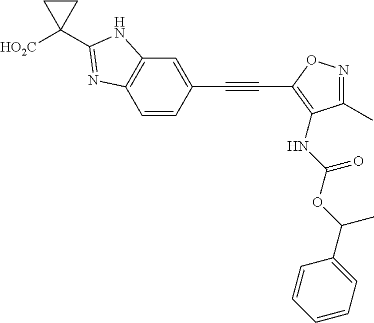 [SMILES: Cc1noc(C#Cc2ccc3nc(C4(C(=O)O)CC4)[nH]c3c2)c1NC(=O)OC(C)c1ccccc1]

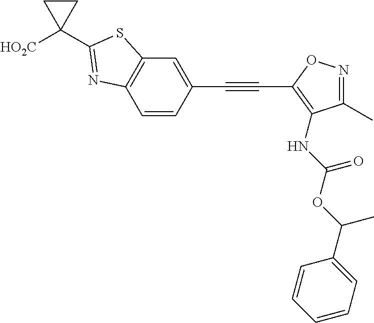 [SMILES: Cc1noc(C#Cc2ccc3nc(C4(C(=O)O)CC4)sc3c2)c1NC(=O)OC(C)c1ccccc1]

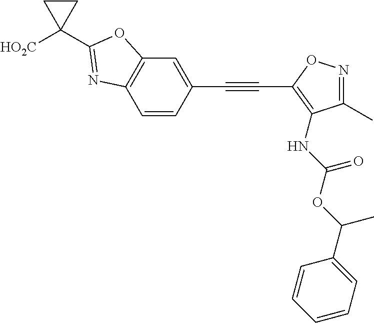 [SMILES: Cc1noc(C#Cc2ccc3nc(C4(C(=O)O)CC4)oc3c2)c1NC(=O)OC(C)c1ccccc1]

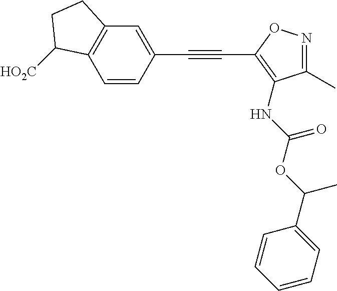 [SMILES: Cc1noc(C#Cc2ccc3c(c2)CCC3C(=O)O)c1NC(=O)OC(C)c1ccccc1]

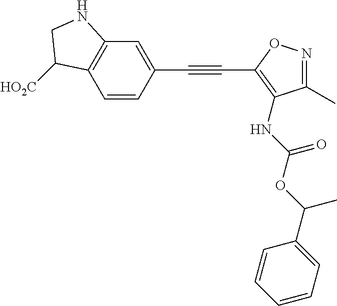 [SMILES: Cc1noc(C#Cc2ccc3c(c2)NCC3C(=O)O)c1NC(=O)OC(C)c1ccccc1]

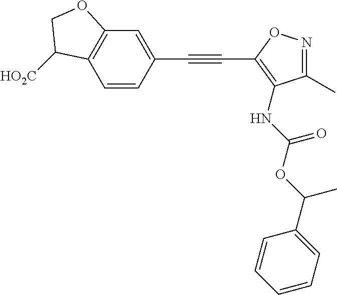 [SMILES: Cc1noc(C#Cc2ccc3c(c2)OCC3C(=O)O)c1NC(=O)OC(C)c1ccccc1]

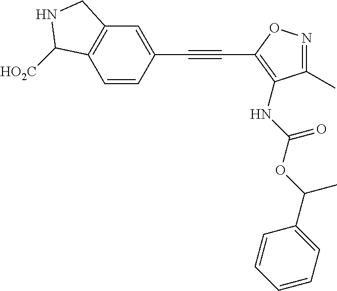 [SMILES: Cc1noc(C#Cc2ccc3c(c2)CNC3C(=O)O)c1NC(=O)OC(C)c1ccccc1]

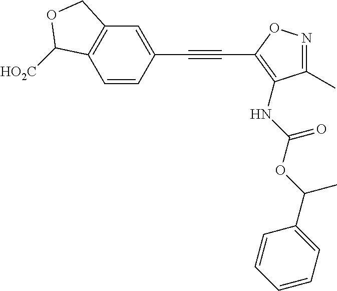 [SMILES: Cc1noc(C#Cc2ccc3c(c2)COC3C(=O)O)c1NC(=O)OC(C)c1ccccc1]

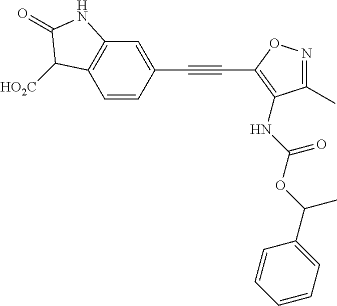 [SMILES: Cc1noc(C#Cc2ccc3c(c2)NC(=O)C3C(=O)O)c1NC(=O)OC(C)c1ccccc1]

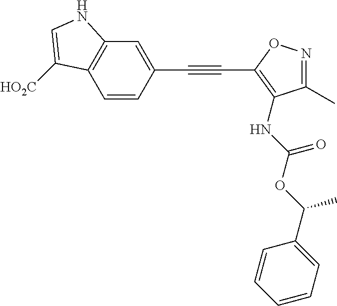 [SMILES: Cc1noc(C#Cc2ccc3c(C(=O)O)c[nH]c3c2)c1NC(=O)O[C@H](C)c1ccccc1]

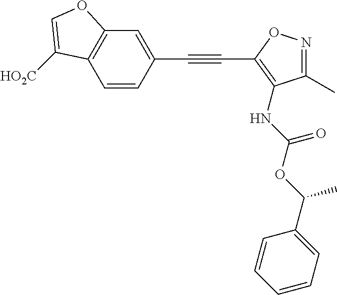 [SMILES: Cc1noc(C#Cc2ccc3c(C(=O)O)coc3c2)c1NC(=O)O[C@H](C)c1ccccc1]

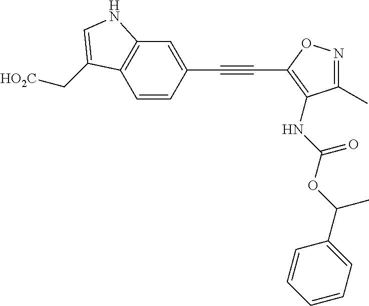 [SMILES: Cc1noc(C#Cc2ccc3c(CC(=O)O)c[nH]c3c2)c1NC(=O)OC(C)c1ccccc1]

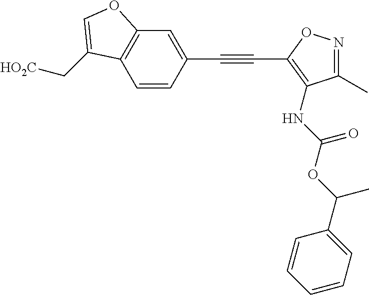 [SMILES: Cc1noc(C#Cc2ccc3c(CC(=O)O)coc3c2)c1NC(=O)OC(C)c1ccccc1]

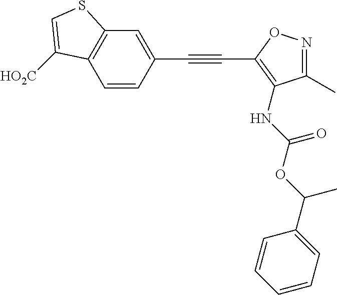 [SMILES: Cc1noc(C#Cc2ccc3c(C(=O)O)csc3c2)c1NC(=O)OC(C)c1ccccc1]

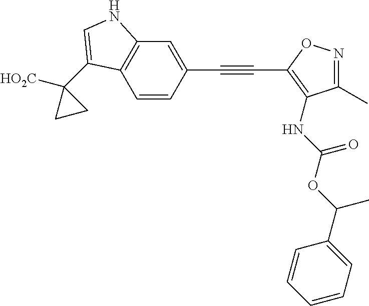 [SMILES: Cc1noc(C#Cc2ccc3c(C4(C(=O)O)CC4)c[nH]c3c2)c1NC(=O)OC(C)c1ccccc1]

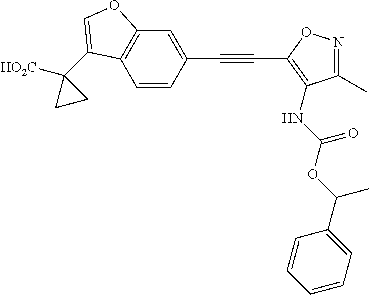 [SMILES: Cc1noc(C#Cc2ccc3c(C4(C(=O)O)CC4)coc3c2)c1NC(=O)OC(C)c1ccccc1]

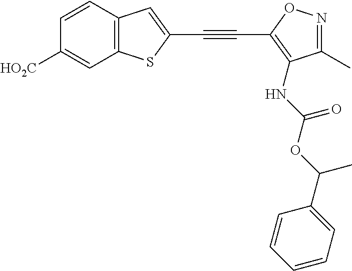 [SMILES: Cc1noc(C#Cc2cc3ccc(C(=O)O)cc3s2)c1NC(=O)OC(C)c1ccccc1]

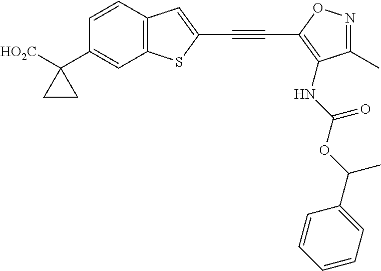 [SMILES: Cc1noc(C#Cc2cc3ccc(C4(C(=O)O)CC4)cc3s2)c1NC(=O)OC(C)c1ccccc1]

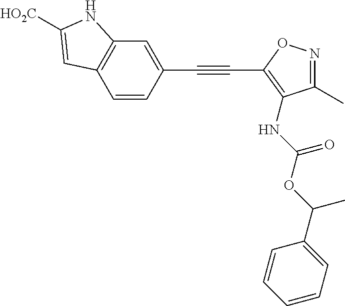 [SMILES: Cc1noc(C#Cc2ccc3cc(C(=O)O)[nH]c3c2)c1NC(=O)OC(C)c1ccccc1]

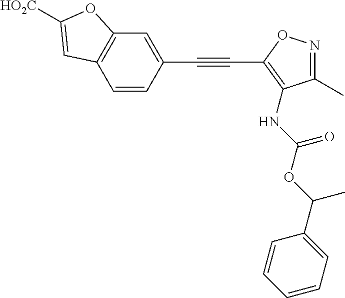 [SMILES: Cc1noc(C#Cc2ccc3cc(C(=O)O)oc3c2)c1NC(=O)OC(C)c1ccccc1]

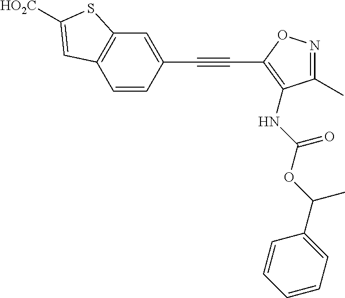 [SMILES: Cc1noc(C#Cc2ccc3cc(C(=O)O)sc3c2)c1NC(=O)OC(C)c1ccccc1]

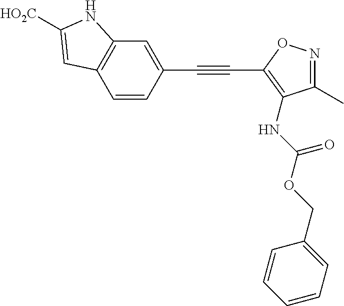 [SMILES: Cc1noc(C#Cc2ccc3cc(C(=O)O)[nH]c3c2)c1NC(=O)OCc1ccccc1]

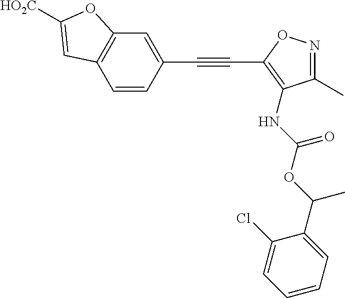 [SMILES: Cc1noc(C#Cc2ccc3cc(C(=O)O)oc3c2)c1NC(=O)OC(C)c1ccccc1Cl]

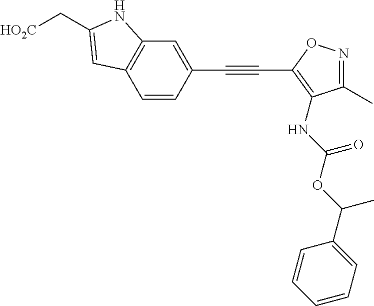 [SMILES: Cc1noc(C#Cc2ccc3cc(CC(=O)O)[nH]c3c2)c1NC(=O)OC(C)c1ccccc1]

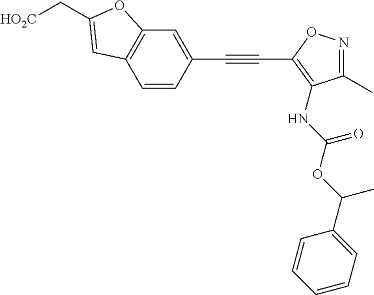 [SMILES: Cc1noc(C#Cc2ccc3cc(CC(=O)O)oc3c2)c1NC(=O)OC(C)c1ccccc1]

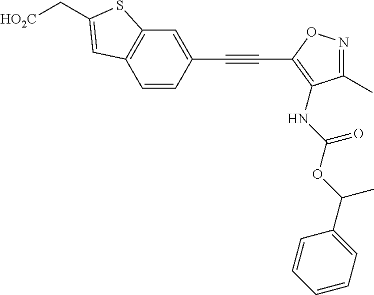 [SMILES: Cc1noc(C#Cc2ccc3cc(CC(=O)O)sc3c2)c1NC(=O)OC(C)c1ccccc1]

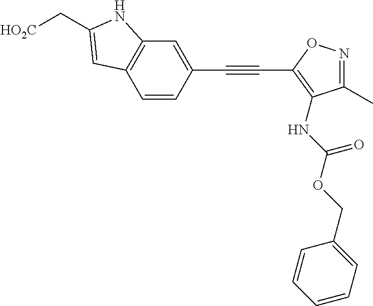 [SMILES: Cc1noc(C#Cc2ccc3cc(CC(=O)O)[nH]c3c2)c1NC(=O)OCc1ccccc1]

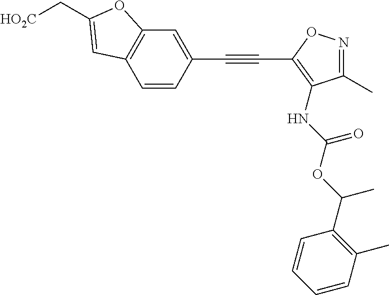 [SMILES: Cc1ccccc1C(C)OC(=O)Nc1c(C)noc1C#Cc1ccc2cc(CC(=O)O)oc2c1]

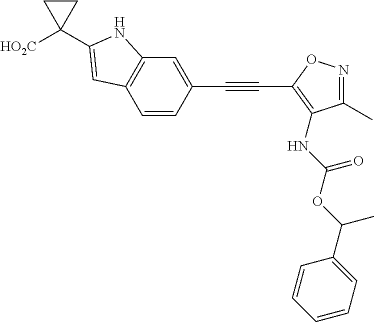 [SMILES: Cc1noc(C#Cc2ccc3cc(C4(C(=O)O)CC4)[nH]c3c2)c1NC(=O)OC(C)c1ccccc1]

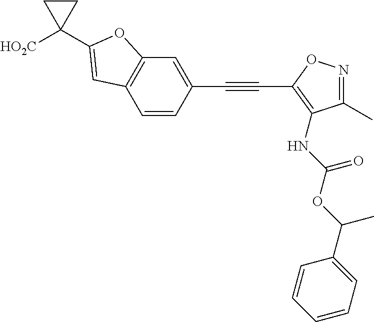 [SMILES: Cc1noc(C#Cc2ccc3cc(C4(C(=O)O)CC4)oc3c2)c1NC(=O)OC(C)c1ccccc1]

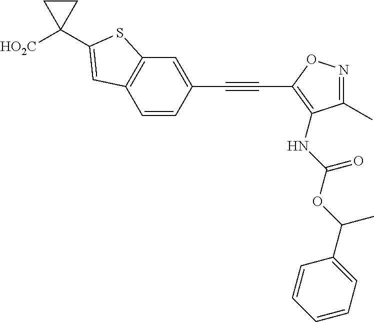 [SMILES: Cc1noc(C#Cc2ccc3cc(C4(C(=O)O)CC4)sc3c2)c1NC(=O)OC(C)c1ccccc1]

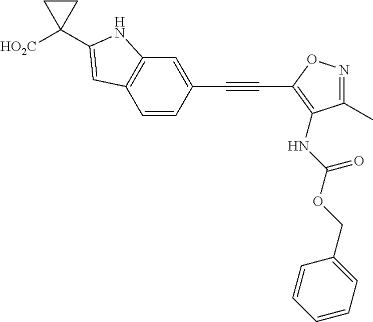 [SMILES: Cc1noc(C#Cc2ccc3cc(C4(C(=O)O)CC4)[nH]c3c2)c1NC(=O)OCc1ccccc1]

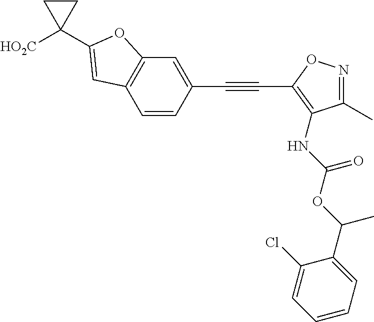 [SMILES: Cc1noc(C#Cc2ccc3cc(C4(C(=O)O)CC4)oc3c2)c1NC(=O)OC(C)c1ccccc1Cl]

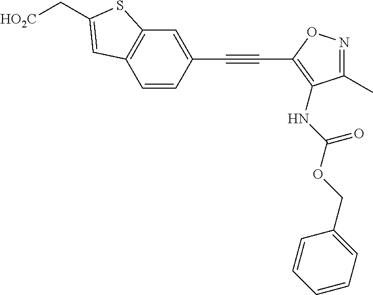 [SMILES: Cc1noc(C#Cc2ccc3cc(CC(=O)O)sc3c2)c1NC(=O)OCc1ccccc1]

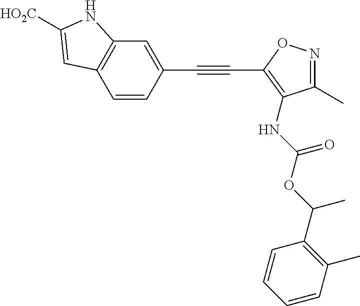 [SMILES: Cc1ccccc1C(C)OC(=O)Nc1c(C)noc1C#Cc1ccc2cc(C(=O)O)[nH]c2c1]

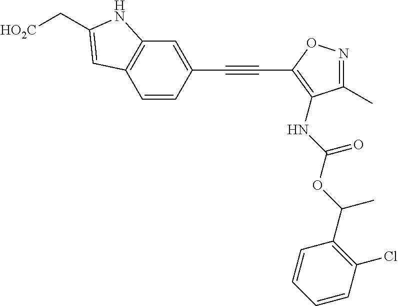 [SMILES: Cc1noc(C#Cc2ccc3cc(CC(=O)O)[nH]c3c2)c1NC(=O)OC(C)c1ccccc1Cl]

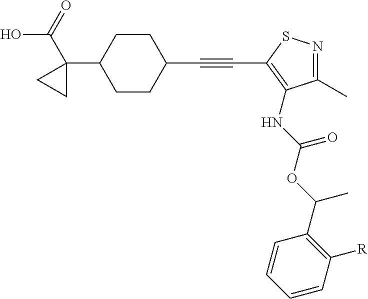

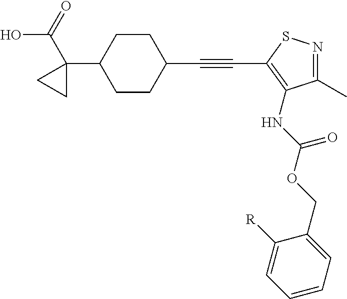

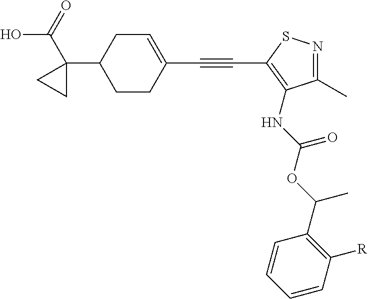

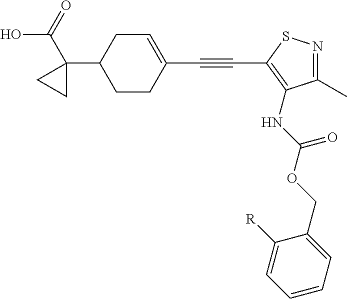

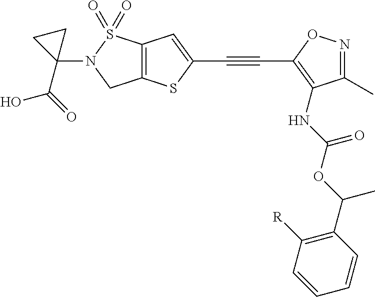

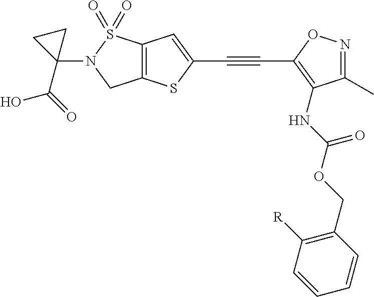

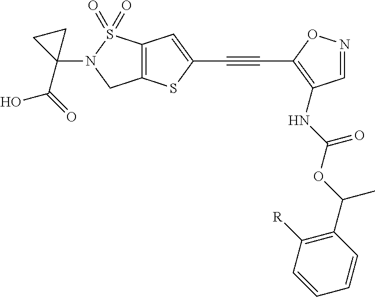

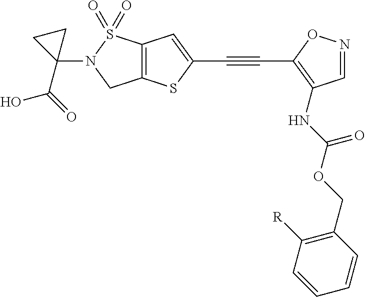

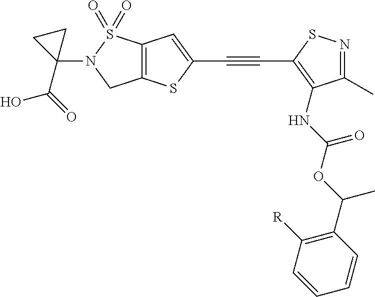

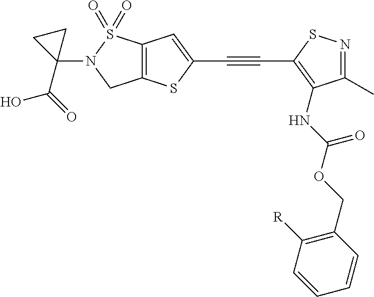

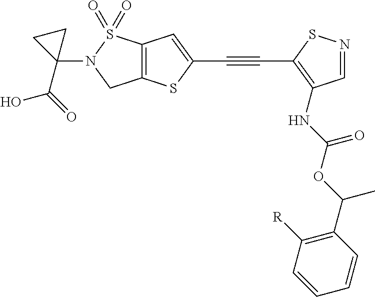

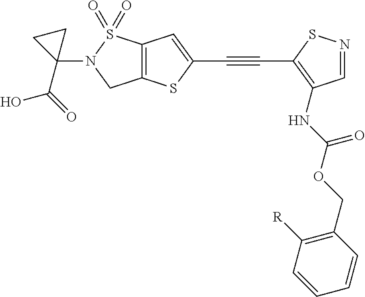

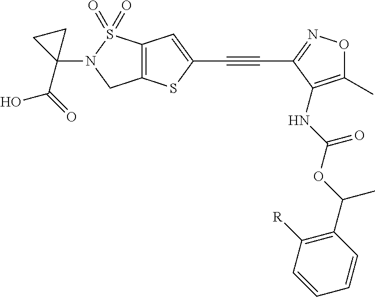

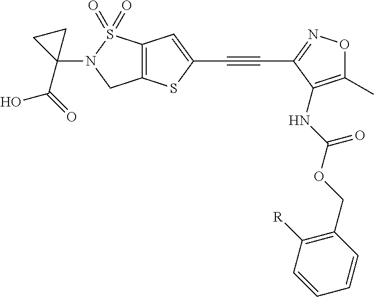

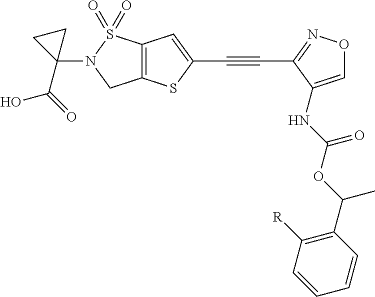

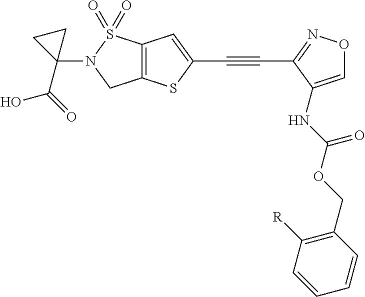

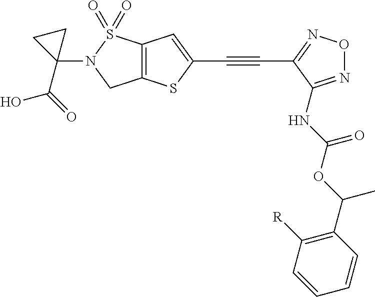

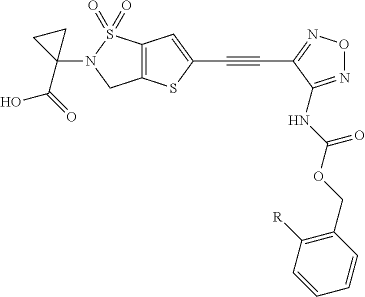

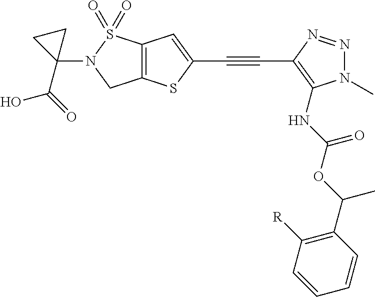

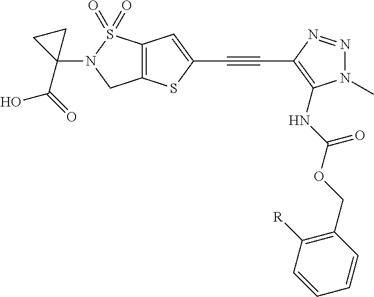

R = H, Me and Cl

In some embodiments, compounds of Formula (VI) are selected from the following compounds as listed in Table 6.

TABLE 6

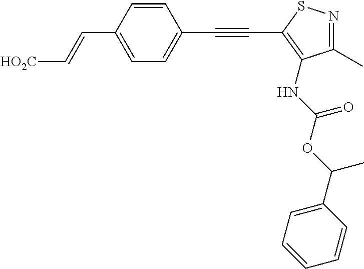 [SMILES: Cc1nsc(C#Cc2ccc(/C=C/C(=O)O)cc2)c1NC(=O)OC(C)c1ccccc1]

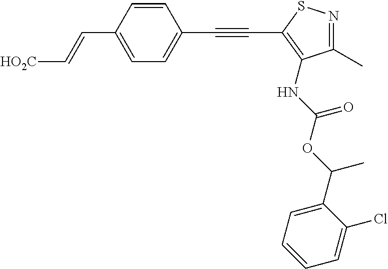 [SMILES: Cc1nsc(C#Cc2ccc(/C=C/C(=O)O)cc2)c1NC(=O)OC(C)c1ccccc1Cl]

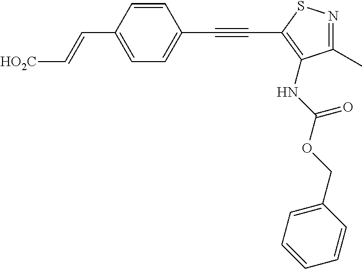 [SMILES: Cc1nsc(C#Cc2ccc(/C=C/C(=O)O)cc2)c1NC(=O)OCc1ccccc1]

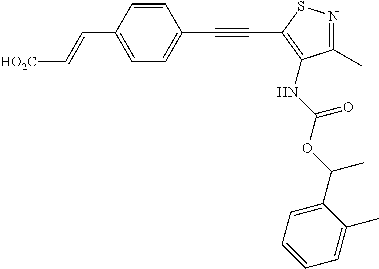 [SMILES: Cc1ccccc1C(C)OC(=O)Nc1c(C)nsc1C#Cc1ccc(/C=C/C(=O)O)cc1]

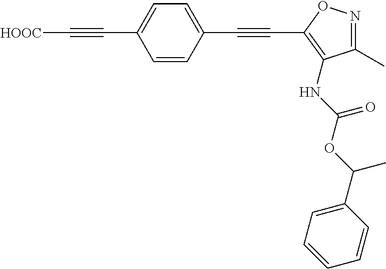 [SMILES: Cc1noc(C#Cc2ccc(C#CC(=O)O)cc2)c1NC(=O)OC(C)c1ccccc1]

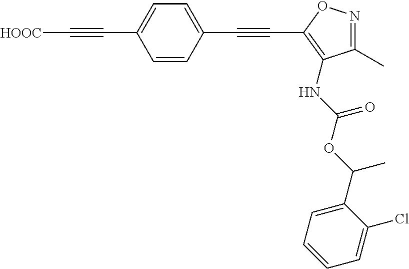 [SMILES: Cc1noc(C#Cc2ccc(C#CC(=O)O)cc2)c1NC(=O)OC(C)c1ccccc1Cl]

In some embodiments, compounds of Formula (VII) are selected from the following compounds as listed in Table 7.

TABLE 7

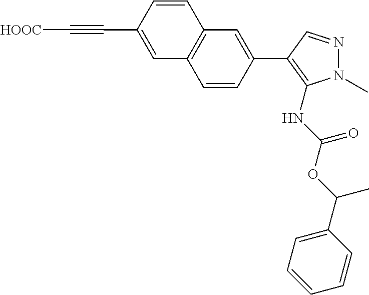 [SMILES: CC(OC(=O)Nc1c(-c2ccc3cc(C#CC(=O)O)ccc3c2)cnn1C)c1ccccc1]

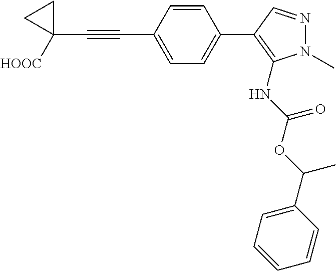 [SMILES: CC(OC(=O)Nc1c(-c2ccc(C#CC3(C(=O)O)CC3)cc2)cnn1C)c1ccccc1]

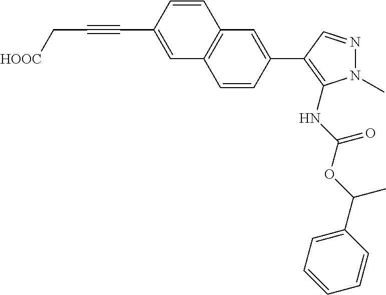 [SMILES: CC(OC(=O)Nc1c(-c2ccc3cc(C#CCC(=O)O)ccc3c2)cnn1C)c1ccccc1]

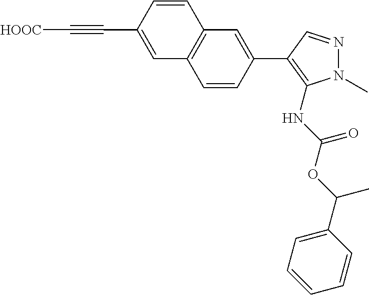 [SMILES: CC(OC(=O)Nc1c(-c2ccc3cc(C#CC(=O)O)ccc3c2)cnn1C)c1ccccc1]

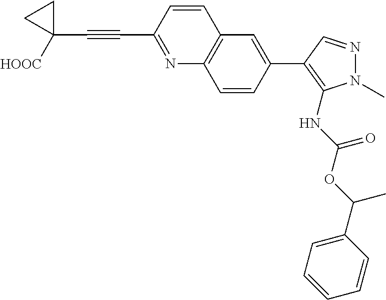 [SMILES: CC(OC(=O)Nc1c(-c2ccc3nc(C#CC4(C(=O)O)CC4)ccc3c2)cnn1C)c1ccccc1]

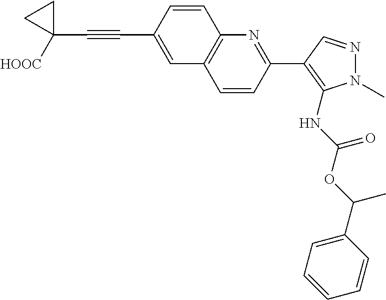 [SMILES: CC(OC(=O)Nc1c(-c2ccc3cc(C#CC4(C(=O)O)CC4)ccc3n2)cnn1C)c1ccccc1]

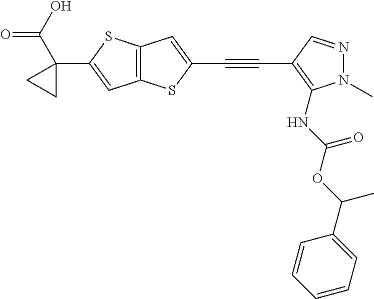 [SMILES: CC(OC(=O)Nc1c(C#Cc2cc3sc(C4(C(=O)O)CC4)cc3s2)cnn1C)c1ccccc1]

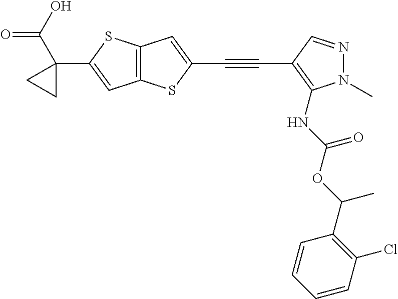 [SMILES: CC(OC(=O)Nc1c(C#Cc2cc3sc(C4(C(=O)O)CC4)cc3s2)cnn1C)c1ccccc1Cl]

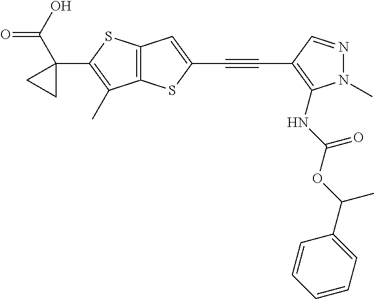 [SMILES: Cc1c(C2(C(=O)O)CC2)sc2cc(C#Cc3cnn(C)c3NC(=O)OC(C)c3ccccc3)sc12]

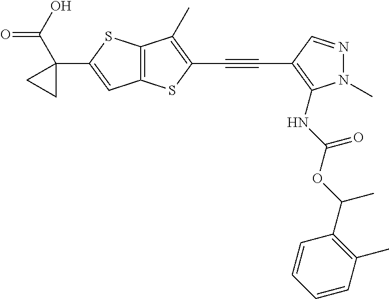 [SMILES: Cc1ccccc1C(C)OC(=O)Nc1c(C#Cc2sc3cc(C4(C(=O)O)CC4)sc3c2C)cnn1C]

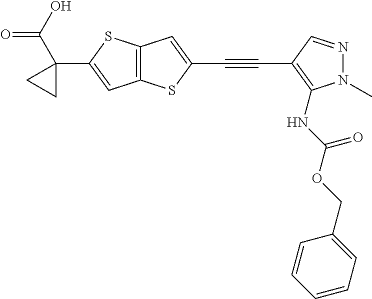 [SMILES: Cn1ncc(C#Cc2cc3sc(C4(C(=O)O)CC4)cc3s2)c1NC(=O)OCc1ccccc1]

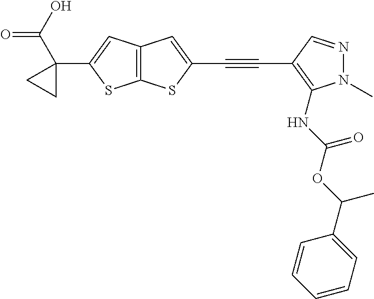 [SMILES: CC(OC(=O)Nc1c(C#Cc2cc3cc(C4(C(=O)O)CC4)sc3s2)cnn1C)c1ccccc1]

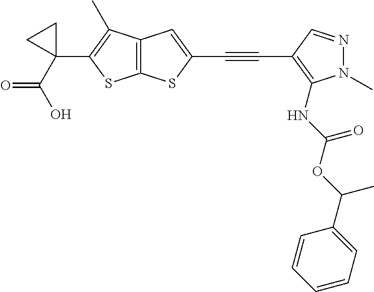 [SMILES: Cc1c(C2(C(=O)O)CC2)sc2sc(C#Cc3cnn(C)c3NC(=O)OC(C)c3ccccc3)cc12]

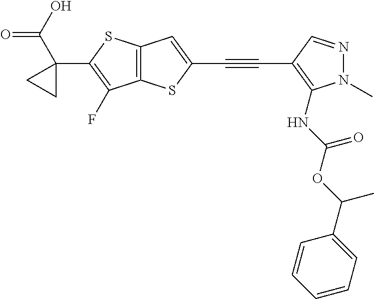 [SMILES: CC(OC(=O)Nc1c(C#Cc2cc3sc(C4(C(=O)O)CC4)c(F)c3s2)cnn1C)c1ccccc1]

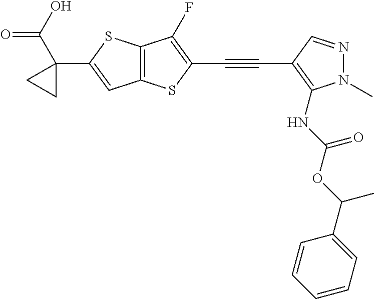 [SMILES: CC(OC(=O)Nc1c(C#Cc2sc3cc(C4(C(=O)O)CC4)sc3c2F)cnn1C)c1ccccc1]

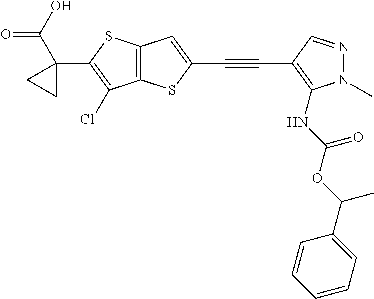 [SMILES: CC(OC(=O)Nc1c(C#Cc2cc3sc(C4(C(=O)O)CC4)c(Cl)c3s2)cnn1C)c1ccccc1]

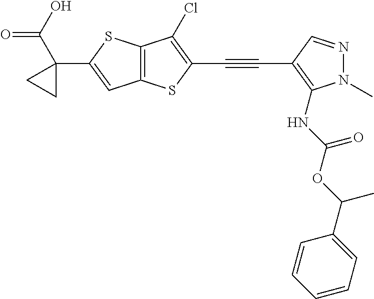 [SMILES: CC(OC(=O)Nc1c(C#Cc2sc3cc(C4(C(=O)O)CC4)sc3c2Cl)cnn1C)c1ccccc1]

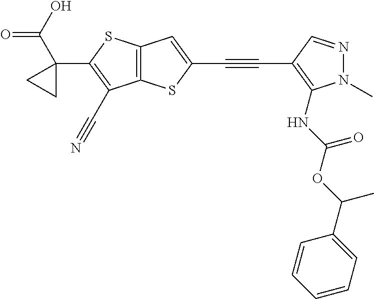 [SMILES: CC(OC(=O)Nc1c(C#Cc2cc3sc(C4(C(=O)O)CC4)c(C#N)c3s2)cnn1C)c1ccccc1]

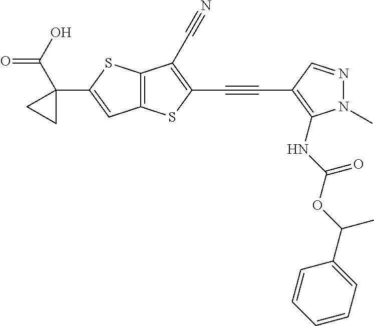 [SMILES: CC(OC(=O)Nc1c(C#Cc2sc3cc(C4(C(=O)O)CC4)sc3c2C#N)cnn1C)c1ccccc1]

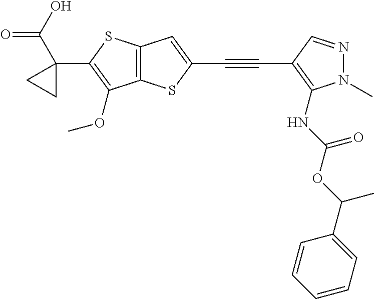 [SMILES: COc1c(C2(C(=O)O)CC2)sc2cc(C#Cc3cnn(C)c3NC(=O)OC(C)c3ccccc3)sc12]

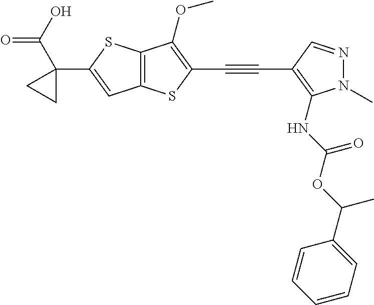 [SMILES: COc1c(C#Cc2cnn(C)c2NC(=O)OC(C)c2ccccc2)sc2cc(C3(C(=O)O)CC3)sc12]

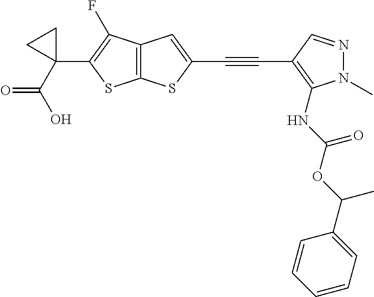 [SMILES: CC(OC(=O)Nc1c(C#Cc2cc3c(F)c(C4(C(=O)O)CC4)sc3s2)cnn1C)c1ccccc1]

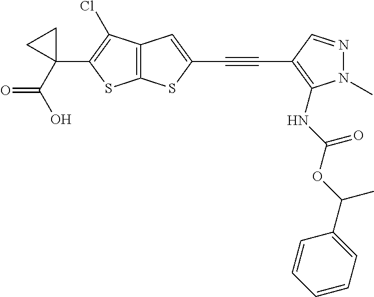 [SMILES: CC(OC(=O)Nc1c(C#Cc2cc3c(Cl)c(C4(C(=O)O)CC4)sc3s2)cnn1C)c1ccccc1]

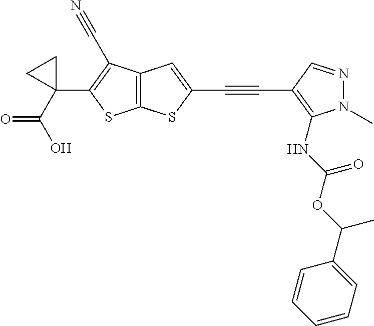 [SMILES: CC(OC(=O)Nc1c(C#Cc2cc3c(C#N)c(C4(C(=O)O)CC4)sc3s2)cnn1C)c1ccccc1]

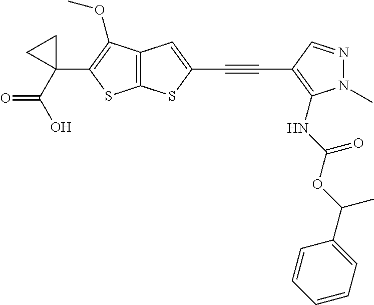 [SMILES: COc1c(C2(C(=O)O)CC2)sc2sc(C#Cc3cnn(C)c3NC(=O)OC(C)c3ccccc3)cc12]

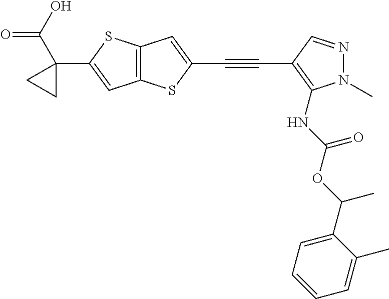 [SMILES: Cc1ccccc1C(C)OC(=O)Nc1c(C#Cc2cc3sc(C4(C(=O)O)CC4)cc3s2)cnn1C]

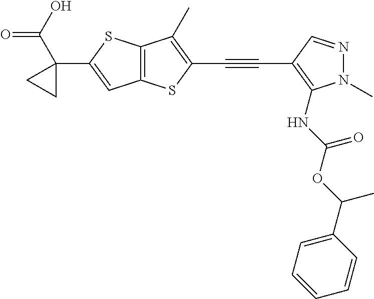 [SMILES: Cc1c(C#Cc2cnn(C)c2NC(=O)OC(C)c2ccccc2)sc2cc(C3(C(=O)O)CC3)sc12]

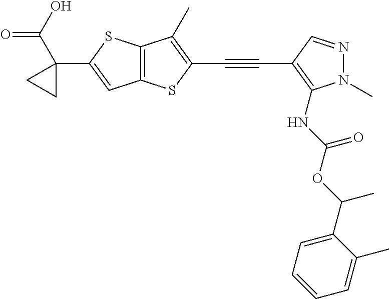 [SMILES: Cc1ccccc1C(C)OC(=O)Nc1c(C#Cc2sc3cc(C4(C(=O)O)CC4)sc3c2C)cnn1C]

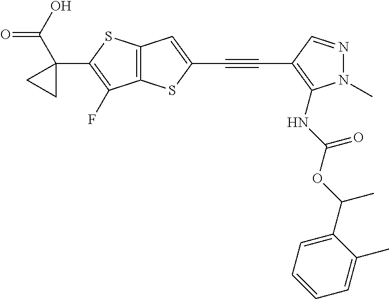 [SMILES: Cc1ccccc1C(C)OC(=O)Nc1c(C#Cc2cc3sc(C4(C(=O)O)CC4)c(F)c3s2)cnn1C]

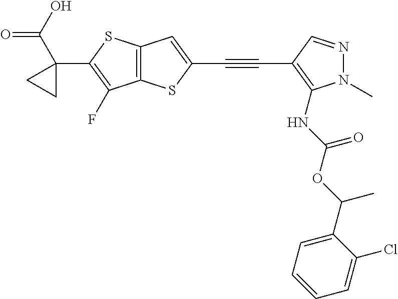 [SMILES: CC(OC(=O)Nc1c(C#Cc2cc3sc(C4(C(=O)O)CC4)c(F)c3s2)cnn1C)c1ccccc1Cl]

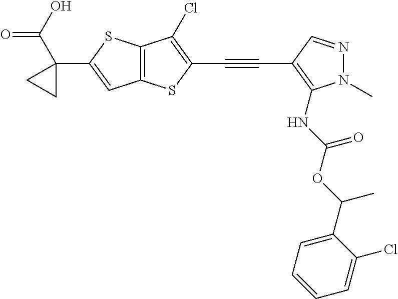 [SMILES: CC(OC(=O)Nc1c(C#Cc2sc3cc(C4(C(=O)O)CC4)sc3c2Cl)cnn1C)c1ccccc1Cl]

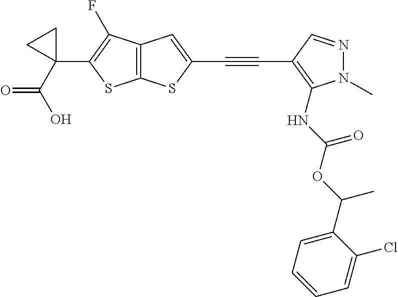 [SMILES: CC(OC(=O)Nc1c(C#Cc2cc3c(F)c(C4(C(=O)O)CC4)sc3s2)cnn1C)c1ccccc1Cl]

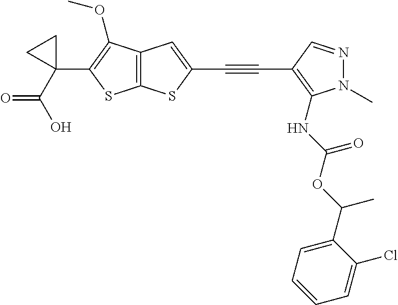 [SMILES: COc1c(C2(C(=O)O)CC2)sc2sc(C#Cc3cnn(C)c3NC(=O)OC(C)c3ccccc3Cl)cc12]

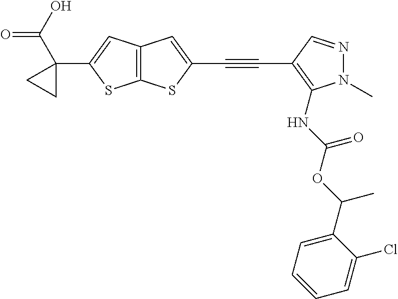 [SMILES: CC(OC(=O)Nc1c(C#Cc2cc3cc(C4(C(=O)O)CC4)sc3s2)cnn1C)c1ccccc1Cl]

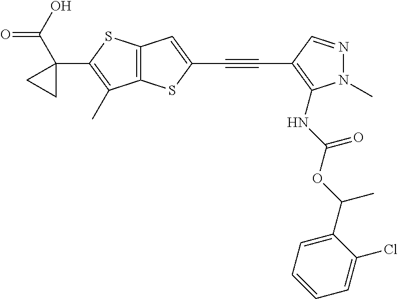 [SMILES: Cc1c(C2(C(=O)O)CC2)sc2cc(C#Cc3cnn(C)c3NC(=O)OC(C)c3ccccc3Cl)sc12]

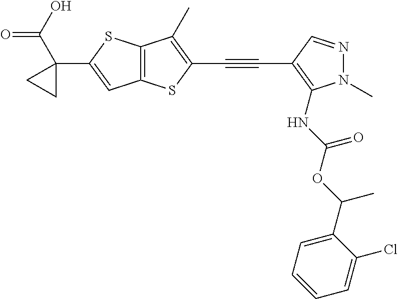 [SMILES: Cc1c(C#Cc2cnn(C)c2NC(=O)OC(C)c2ccccc2Cl)sc2cc(C3(C(=O)O)CC3)sc12]

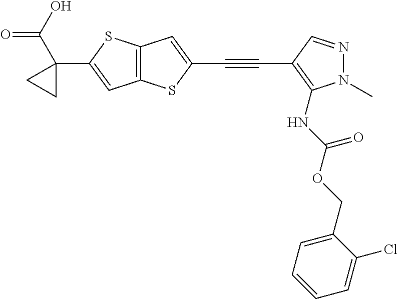 [SMILES: Cn1ncc(C#Cc2cc3sc(C4(C(=O)O)CC4)cc3s2)c1NC(=O)OCc1ccccc1Cl]

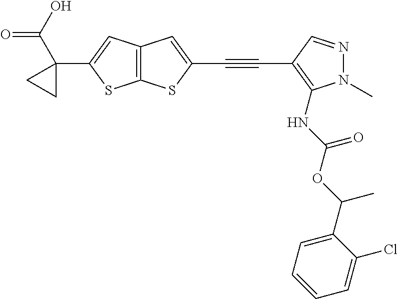 [SMILES: CC(OC(=O)Nc1c(C#Cc2cc3cc(C4(C(=O)O)CC4)sc3s2)cnn1C)c1ccccc1Cl]

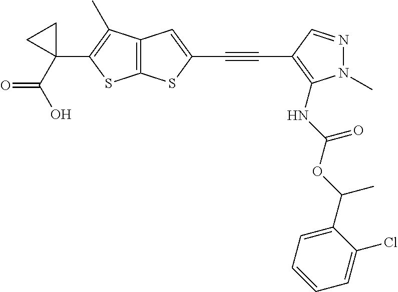 [SMILES: Cc1c(C2(C(=O)O)CC2)sc2sc(C#Cc3cnn(C)c3NC(=O)OC(C)c3ccccc3Cl)cc12]

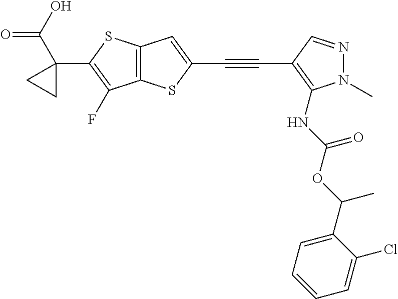 [SMILES: CC(OC(=O)Nc1c(C#Cc2cc3sc(C4(C(=O)O)CC4)c(F)c3s2)cnn1C)c1ccccc1Cl]

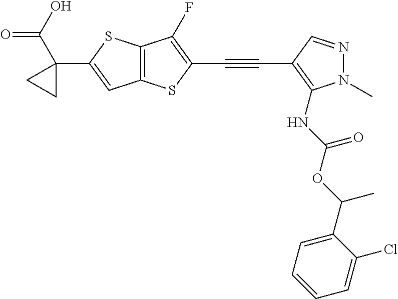 [SMILES: CC(OC(=O)Nc1c(C#Cc2sc3cc(C4(C(=O)O)CC4)sc3c2F)cnn1C)c1ccccc1Cl]

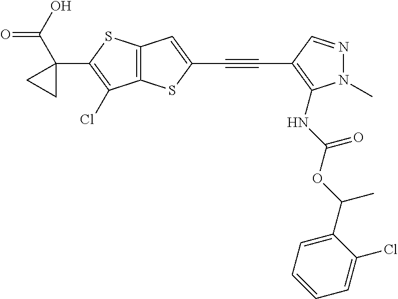 [SMILES: CC(OC(=O)Nc1c(C#Cc2cc3sc(C4(C(=O)O)CC4)c(Cl)c3s2)cnn1C)c1ccccc1Cl]

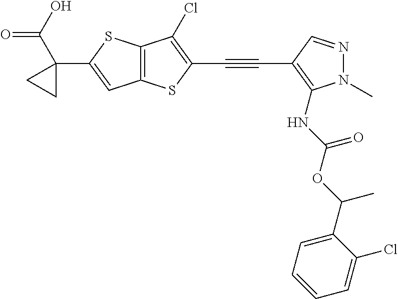 [SMILES: CC(OC(=O)Nc1c(C#Cc2sc3cc(C4(C(=O)O)CC4)sc3c2Cl)cnn1C)c1ccccc1Cl]

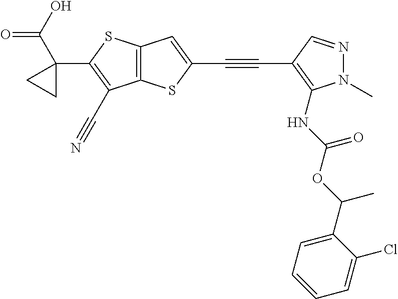 [SMILES: CC(OC(=O)Nc1c(C#Cc2cc3sc(C4(C(=O)O)CC4)c(C#N)c3s2)cnn1C)c1ccccc1Cl]

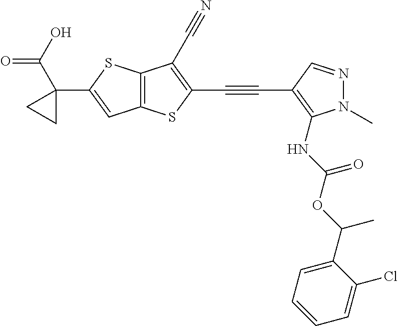 [SMILES: CC(OC(=O)Nc1c(C#Cc2sc3cc(C4(C(=O)O)CC4)sc3c2C#N)cnn1C)c1ccccc1Cl]

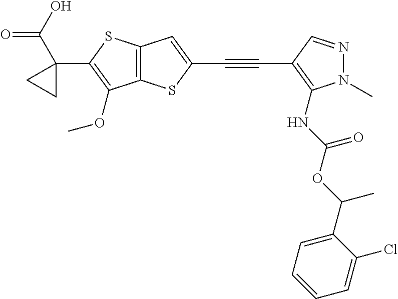 [SMILES: COc1c(C2(C(=O)O)CC2)sc2cc(C#Cc3cnn(C)c3NC(=O)OC(C)c3ccccc3Cl)sc12]

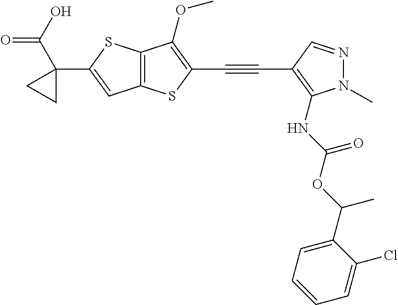 [SMILES: COc1c(C#Cc2cnn(C)c2NC(=O)OC(C)c2ccccc2Cl)sc2cc(C3(C(=O)O)CC3)sc12]

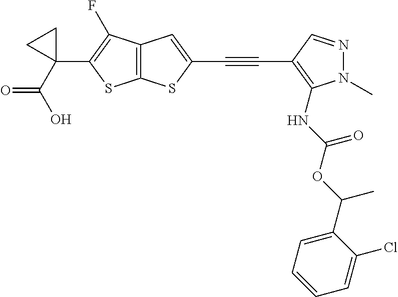 [SMILES: CC(OC(=O)Nc1c(C#Cc2cc3c(F)c(C4(C(=O)O)CC4)sc3s2)cnn1C)c1ccccc1Cl]

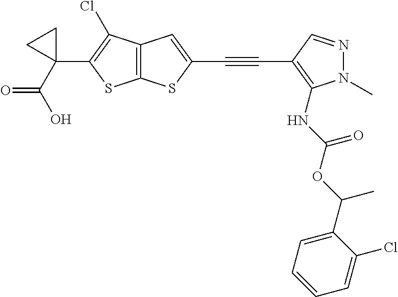 [SMILES: CC(OC(=O)Nc1c(C#Cc2cc3c(Cl)c(C4(C(=O)O)CC4)sc3s2)cnn1C)c1ccccc1Cl]

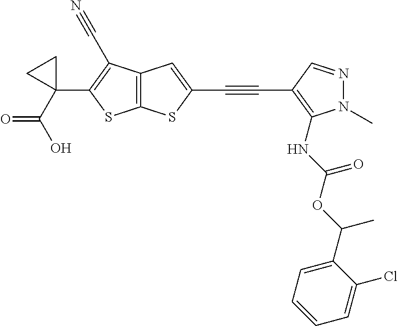 [SMILES: CC(OC(=O)Nc1c(C#Cc2cc3c(C#N)c(C4(C(=O)O)CC4)sc3s2)cnn1C)c1ccccc1Cl]

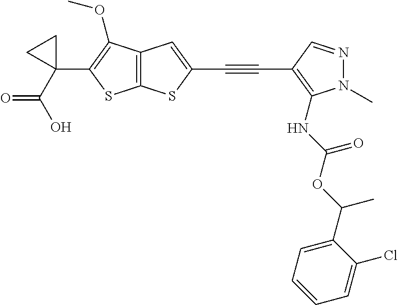 [SMILES: COc1c(C2(C(=O)O)CC2)sc2sc(C#Cc3cnn(C)c3NC(=O)OC(C)c3ccccc3Cl)cc12]

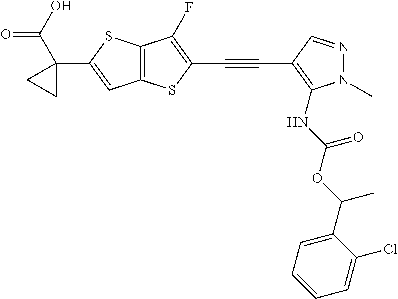 [SMILES: CC(OC(=O)Nc1c(C#Cc2sc3cc(C4(C(=O)O)CC4)sc3c2F)cnn1C)c1ccccc1Cl]

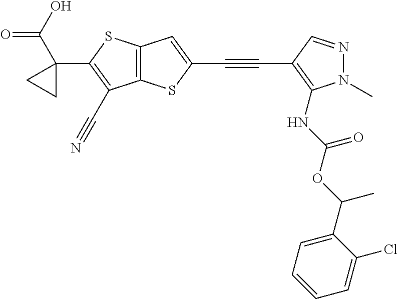 [SMILES: CC(OC(=O)Nc1c(C#Cc2cc3sc(C4(C(=O)O)CC4)c(C#N)c3s2)cnn1C)c1ccccc1Cl]

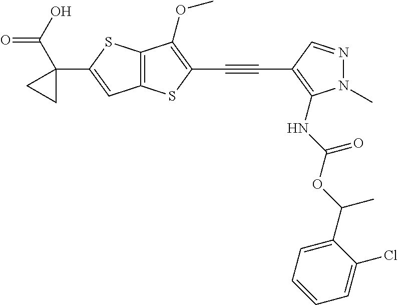 [SMILES: COc1c(C#Cc2cnn(C)c2NC(=O)OC(C)c2ccccc2Cl)sc2cc(C3(C(=O)O)CC3)sc12]

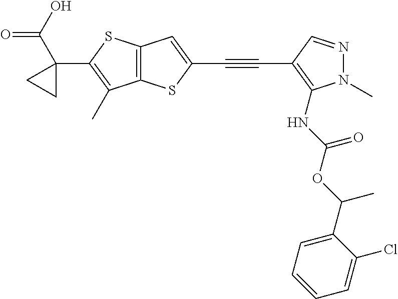 [SMILES: Cc1c(C2(C(=O)O)CC2)sc2cc(C#Cc3cnn(C)c3NC(=O)OC(C)c3ccccc3Cl)sc12]

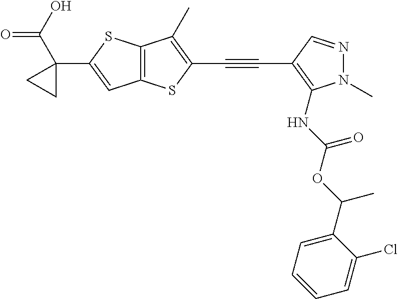 [SMILES: Cc1c(C#Cc2cnn(C)c2NC(=O)OC(C)c2ccccc2Cl)sc2cc(C3(C(=O)O)CC3)sc12]

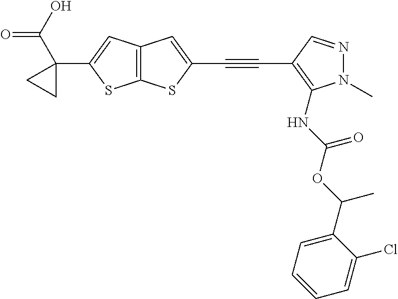 [SMILES: CC(OC(=O)Nc1c(C#Cc2cc3cc(C4(C(=O)O)CC4)sc3s2)cnn1C)c1ccccc1Cl]

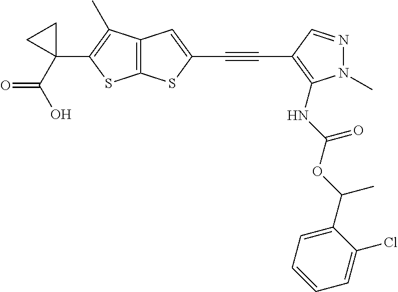 [SMILES: Cc1c(C2(C(=O)O)CC2)sc2sc(C#Cc3cnn(C)c3NC(=O)OC(C)c3ccccc3Cl)cc12]

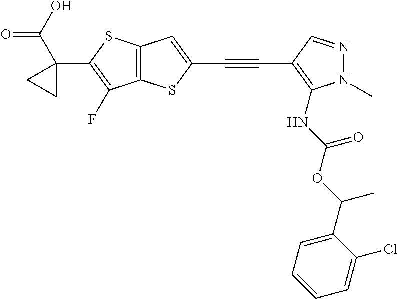 [SMILES: CC(OC(=O)Nc1c(C#Cc2cc3sc(C4(C(=O)O)CC4)c(F)c3s2)cnn1C)c1ccccc1Cl]

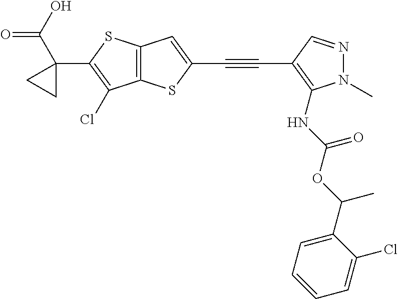 [SMILES: CC(OC(=O)Nc1c(C#Cc2cc3sc(C4(C(=O)O)CC4)c(Cl)c3s2)cnn1C)c1ccccc1Cl]

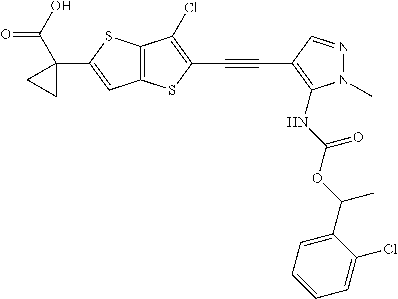 [SMILES: CC(OC(=O)Nc1c(C#Cc2sc3cc(C4(C(=O)O)CC4)sc3c2Cl)cnn1C)c1ccccc1Cl]

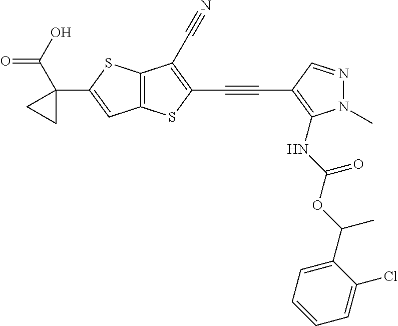 [SMILES: CC(OC(=O)Nc1c(C#Cc2sc3cc(C4(C(=O)O)CC4)sc3c2C#N)cnn1C)c1ccccc1Cl]

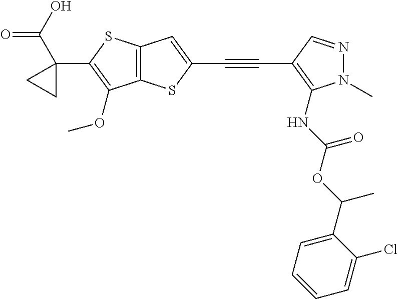 [SMILES: COc1c(C2(C(=O)O)CC2)sc2cc(C#Cc3cnn(C)c3NC(=O)OC(C)c3ccccc3Cl)sc12]

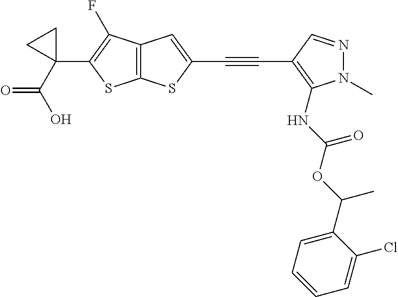 [SMILES: CC(OC(=O)Nc1c(C#Cc2cc3c(F)c(C4(C(=O)O)CC4)sc3s2)cnn1C)c1ccccc1Cl]

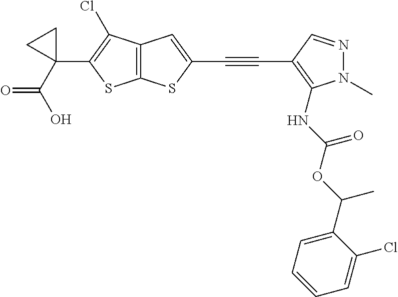 [SMILES: CC(OC(=O)Nc1c(C#Cc2cc3c(Cl)c(C4(C(=O)O)CC4)sc3s2)cnn1C)c1ccccc1Cl]

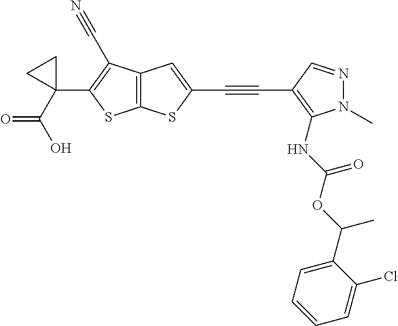 [SMILES: CC(OC(=O)Nc1c(C#Cc2cc3c(C#N)c(C4(C(=O)O)CC4)sc3s2)cnn1C)c1ccccc1Cl]

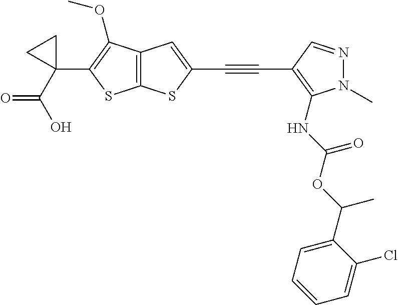 [SMILES: COc1c(C2(C(=O)O)CC2)sc2sc(C#Cc3cnn(C)c3NC(=O)OC(C)c3ccccc3Cl)cc12]

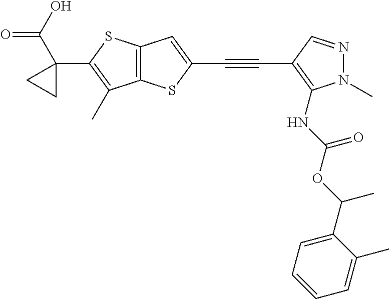 [SMILES: Cc1ccccc1C(C)OC(=O)Nc1c(C#Cc2cc3sc(C4(C(=O)O)CC4)c(C)c3s2)cnn1C]

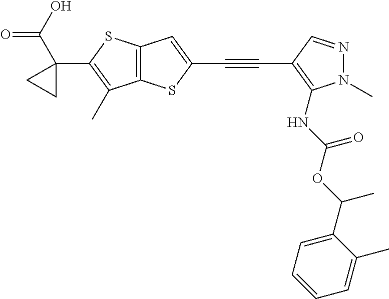 [SMILES: Cc1ccccc1C(C)OC(=O)Nc1c(C#Cc2cc3sc(C4(C(=O)O)CC4)c(C)c3s2)cnn1C]

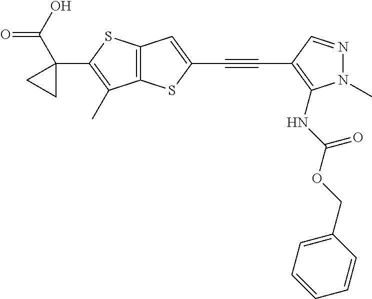 [SMILES: Cc1c(C2(C(=O)O)CC2)sc2cc(C#Cc3cnn(C)c3NC(=O)OCc3ccccc3)sc12]

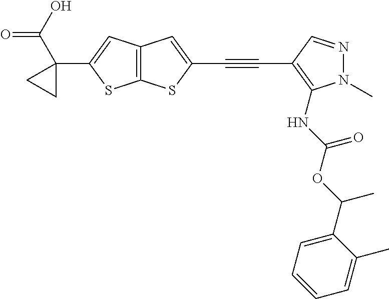 [SMILES: Cc1ccccc1C(C)OC(=O)Nc1c(C#Cc2cc3cc(C4(C(=O)O)CC4)sc3s2)cnn1C]

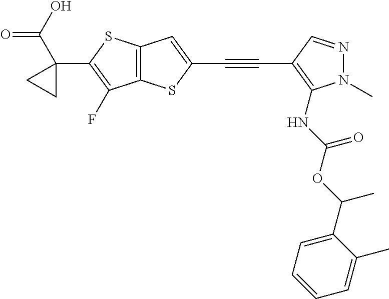 [SMILES: Cc1ccccc1C(C)OC(=O)Nc1c(C#Cc2cc3sc(C4(C(=O)O)CC4)c(F)c3s2)cnn1C]

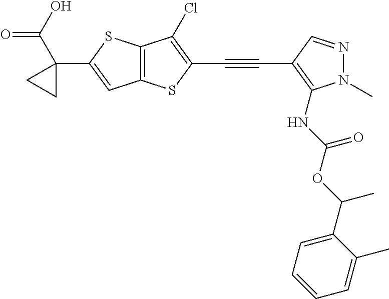 [SMILES: Cc1ccccc1C(C)OC(=O)Nc1c(C#Cc2sc3cc(C4(C(=O)O)CC4)sc3c2Cl)cnn1C]

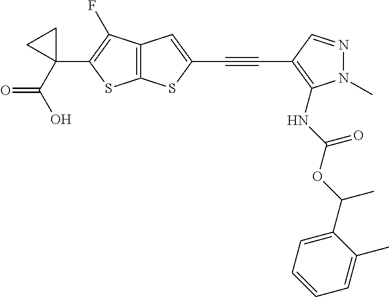 [SMILES: Cc1ccccc1C(C)OC(=O)Nc1c(C#Cc2cc3c(F)c(C4(C(=O)O)CC4)sc3s2)cnn1C]

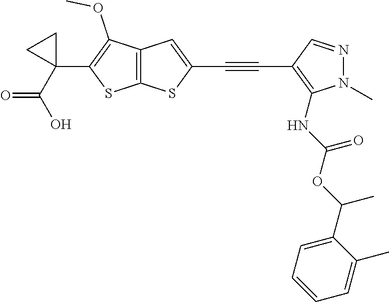 [SMILES: COc1c(C2(C(=O)O)CC2)sc2sc(C#Cc3cnn(C)c3NC(=O)OC(C)c3ccccc3C)cc12]

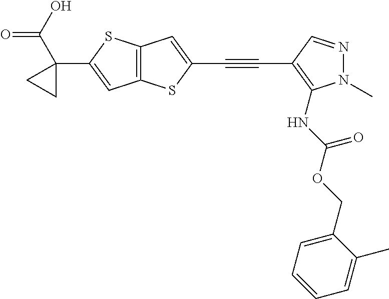 [SMILES: Cc1ccccc1COC(=O)Nc1c(C#Cc2cc3sc(C4(C(=O)O)CC4)cc3s2)cnn1C]

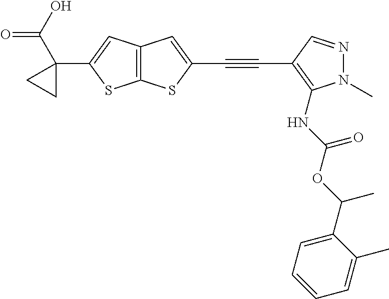 [SMILES: Cc1ccccc1C(C)OC(=O)Nc1c(C#Cc2cc3cc(C4(C(=O)O)CC4)sc3s2)cnn1C]

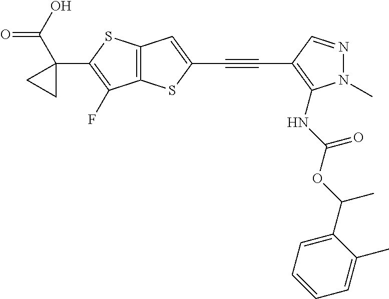 [SMILES: Cc1ccccc1C(C)OC(=O)Nc1c(C#Cc2cc3sc(C4(C(=O)O)CC4)c(F)c3s2)cnn1C]

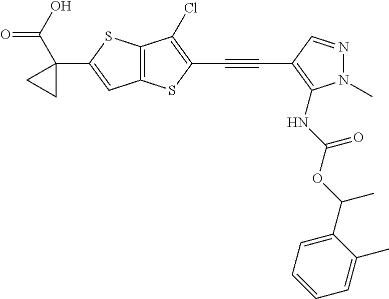 [SMILES: Cc1ccccc1C(C)OC(=O)Nc1c(C#Cc2sc3cc(C4(C(=O)O)CC4)sc3c2Cl)cnn1C]

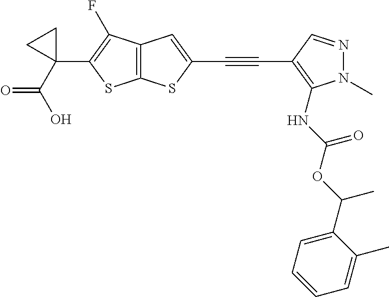 [SMILES: Cc1ccccc1C(C)OC(=O)Nc1c(C#Cc2cc3c(F)c(C4(C(=O)O)CC4)sc3s2)cnn1C]

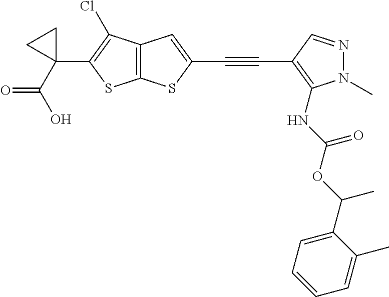 [SMILES: Cc1ccccc1C(C)OC(=O)Nc1c(C#Cc2cc3c(Cl)c(C4(C(=O)O)CC4)sc3s2)cnn1C]

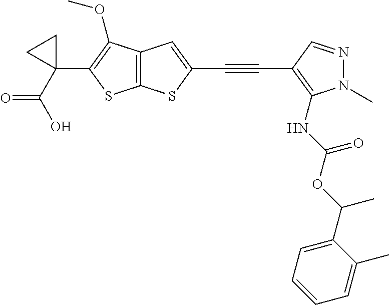 [SMILES: COc1c(C2(C(=O)O)CC2)sc2sc(C#Cc3cnn(C)c3NC(=O)OC(C)c3ccccc3C)cc12]

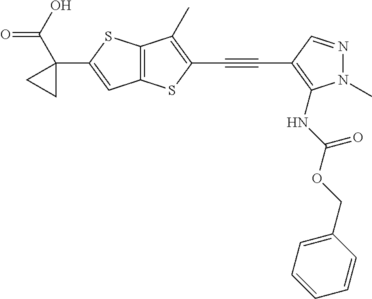 [SMILES: Cc1c(C#Cc2cnn(C)c2NC(=O)OCc2ccccc2)sc2cc(C3(C(=O)O)CC3)sc12]

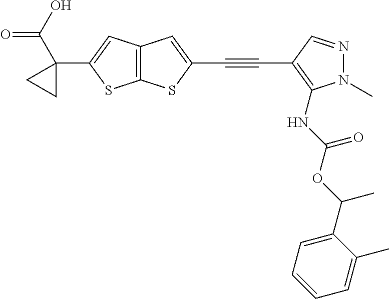 [SMILES: Cc1ccccc1C(C)OC(=O)Nc1c(C#Cc2cc3cc(C4(C(=O)O)CC4)sc3s2)cnn1C]

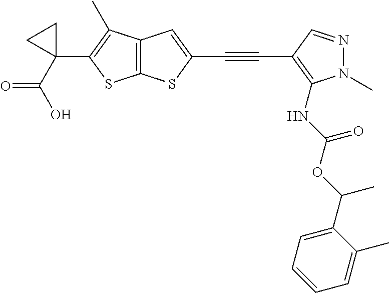 [SMILES: Cc1ccccc1C(C)OC(=O)Nc1c(C#Cc2cc3c(C)c(C4(C(=O)O)CC4)sc3s2)cnn1C]

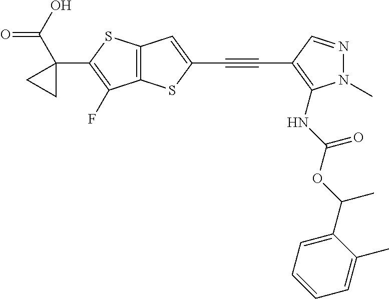 [SMILES: Cc1ccccc1C(C)OC(=O)Nc1c(C#Cc2cc3sc(C4(C(=O)O)CC4)c(F)c3s2)cnn1C]

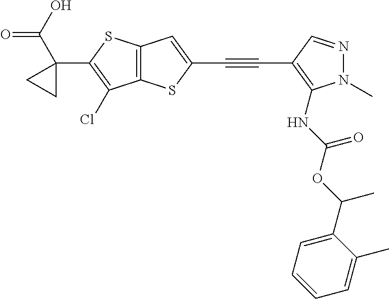 [SMILES: Cc1ccccc1C(C)OC(=O)Nc1c(C#Cc2cc3sc(C4(C(=O)O)CC4)c(Cl)c3s2)cnn1C]

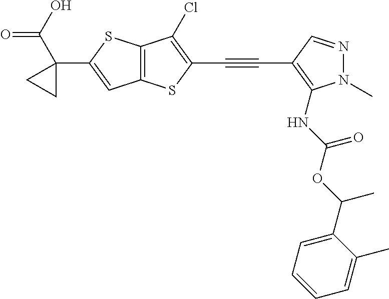 [SMILES: Cc1ccccc1C(C)OC(=O)Nc1c(C#Cc2sc3cc(C4(C(=O)O)CC4)sc3c2Cl)cnn1C]

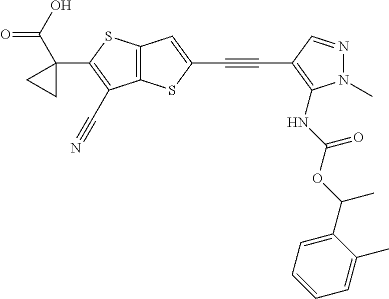 [SMILES: Cc1ccccc1C(C)OC(=O)Nc1c(C#Cc2cc3sc(C4(C(=O)O)CC4)c(C#N)c3s2)cnn1C]

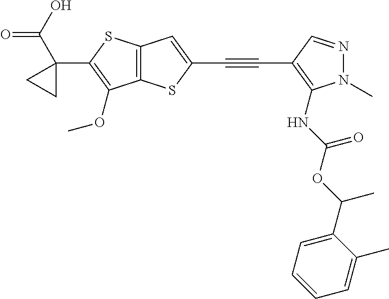 [SMILES: COc1c(C2(C(=O)O)CC2)sc2cc(C#Cc3cnn(C)c3NC(=O)OC(C)c3ccccc3C)sc12]

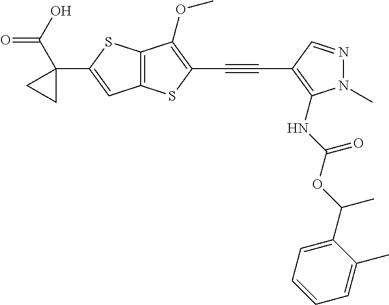 [SMILES: COc1c(C#Cc2cnn(C)c2NC(=O)OC(C)c2ccccc2C)sc2cc(C3(C(=O)O)CC3)sc12]

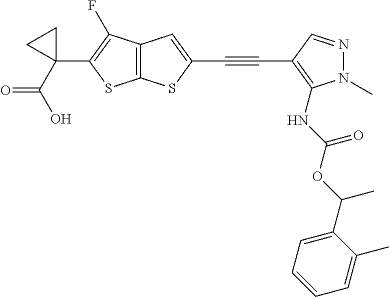 [SMILES: Cc1ccccc1C(C)OC(=O)Nc1c(C#Cc2cc3c(F)c(C4(C(=O)O)CC4)sc3s2)cnn1C]

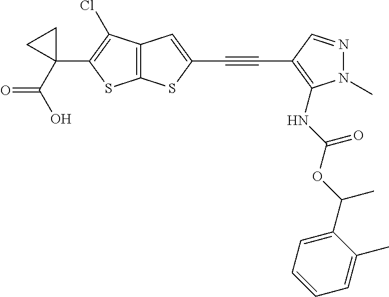 [SMILES: Cc1ccccc1C(C)OC(=O)Nc1c(C#Cc2cc3c(Cl)c(C4(C(=O)O)CC4)sc3s2)cnn1C]

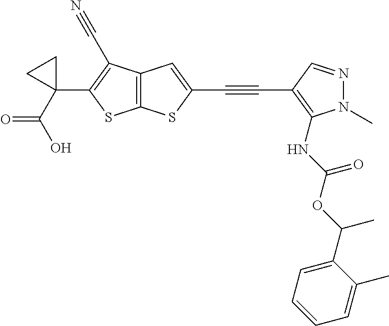 [SMILES: Cc1ccccc1C(C)OC(=O)Nc1c(C#Cc2cc3c(C#N)c(C4(C(=O)O)CC4)sc3s2)cnn1C]

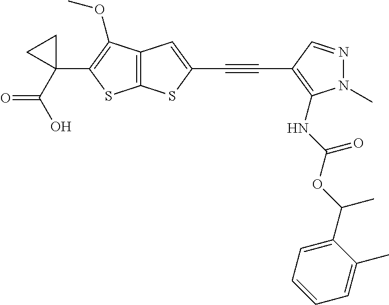 [SMILES: COc1c(C2(C(=O)O)CC2)sc2sc(C#Cc3cnn(C)c3NC(=O)OC(C)c3ccccc3C)cc12]

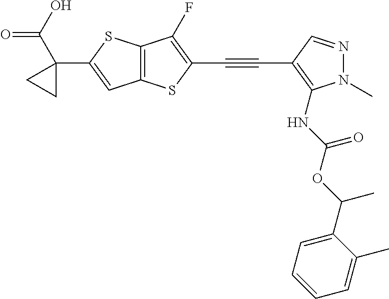 [SMILES: Cc1ccccc1C(C)OC(=O)Nc1c(C#Cc2sc3cc(C4(C(=O)O)CC4)sc3c2F)cnn1C]

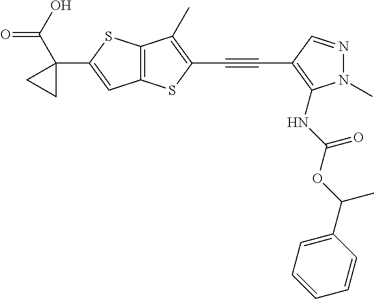 [SMILES: Cc1c(C#Cc2cnn(C)c2NC(=O)OC(C)c2ccccc2)sc2cc(C3(C(=O)O)CC3)sc12]

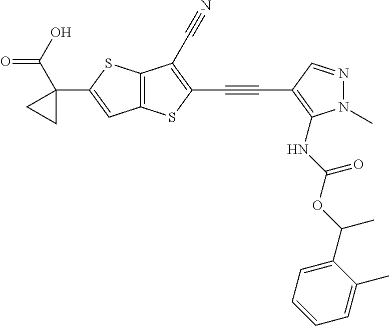 [SMILES: Cc1ccccc1C(C)OC(=O)Nc1c(C#Cc2sc3cc(C4(C(=O)O)CC4)sc3c2C#N)cnn1C]

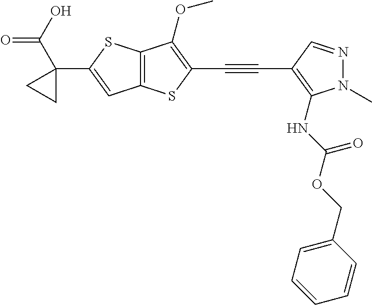 [SMILES: COc1c(C#Cc2cnn(C)c2NC(=O)OCc2ccccc2)sc2cc(C3(C(=O)O)CC3)sc12]

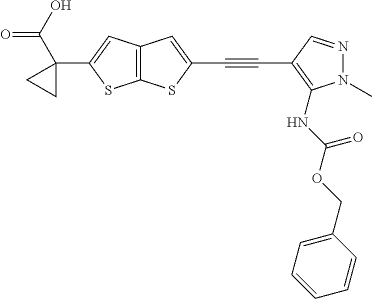 [SMILES: Cn1ncc(C#Cc2cc3cc(C4(C(=O)O)CC4)sc3s2)c1NC(=O)OCc1ccccc1]

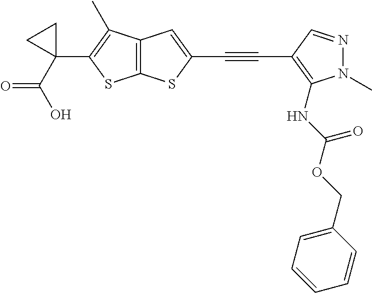 [SMILES: Cc1c(C2(C(=O)O)CC2)sc2sc(C#Cc3cnn(C)c3NC(=O)OCc3ccccc3)cc12]

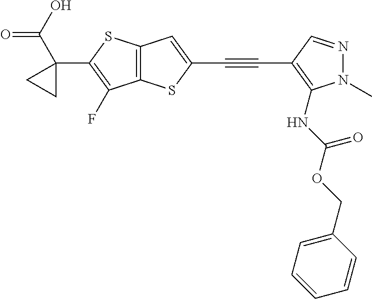 [SMILES: Cn1ncc(C#Cc2cc3sc(C4(C(=O)O)CC4)c(F)c3s2)c1NC(=O)OCc1ccccc1]

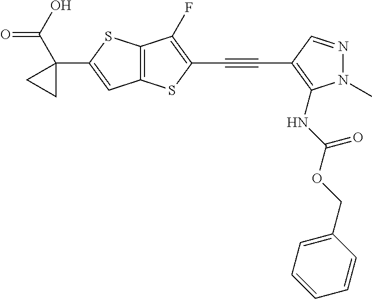 [SMILES: Cn1ncc(C#Cc2sc3cc(C4(C(=O)O)CC4)sc3c2F)c1NC(=O)OCc1ccccc1]

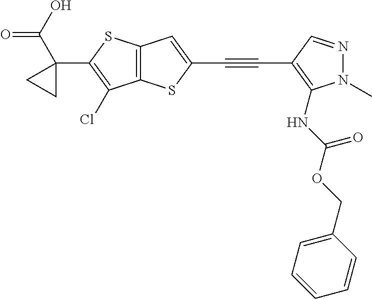 [SMILES: Cn1ncc(C#Cc2cc3sc(C4(C(=O)O)CC4)c(Cl)c3s2)c1NC(=O)OCc1ccccc1]

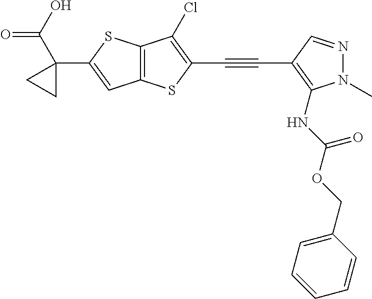 [SMILES: Cn1ncc(C#Cc2sc3cc(C4(C(=O)O)CC4)sc3c2Cl)c1NC(=O)OCc1ccccc1]

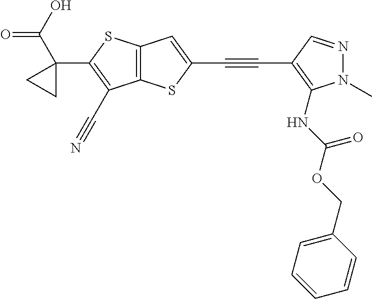 [SMILES: Cn1ncc(C#Cc2cc3sc(C4(C(=O)O)CC4)c(C#N)c3s2)c1NC(=O)OCc1ccccc1]

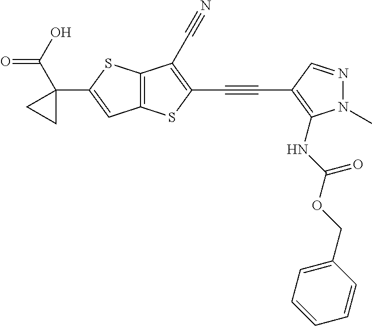 [SMILES: Cn1ncc(C#Cc2sc3cc(C4(C(=O)O)CC4)sc3c2C#N)c1NC(=O)OCc1ccccc1]

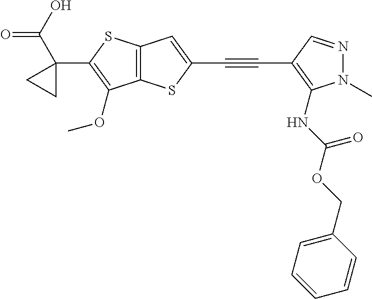 [SMILES: COc1c(C2(C(=O)O)CC2)sc2cc(C#Cc3cnn(C)c3NC(=O)OCc3ccccc3)sc12]

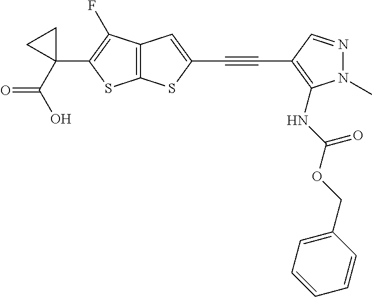 [SMILES: Cn1ncc(C#Cc2cc3c(F)c(C4(C(=O)O)CC4)sc3s2)c1NC(=O)OCc1ccccc1]

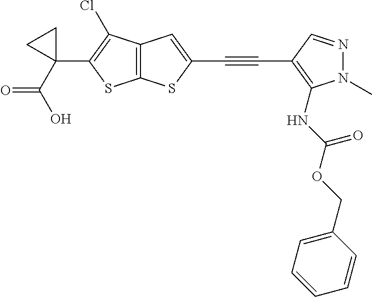 [SMILES: Cn1ncc(C#Cc2cc3c(Cl)c(C4(C(=O)O)CC4)sc3s2)c1NC(=O)OCc1ccccc1]

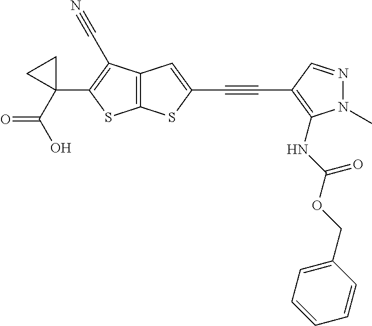 [SMILES: Cn1ncc(C#Cc2cc3c(C#N)c(C4(C(=O)O)CC4)sc3s2)c1NC(=O)OCc1ccccc1]

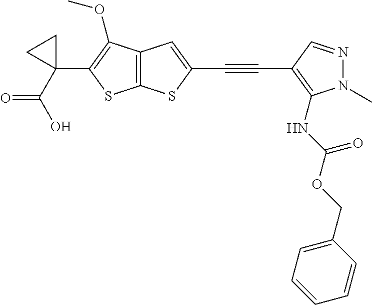 [SMILES: COc1c(C2(C(=O)O)CC2)sc2sc(C#Cc3cnn(C)c3NC(=O)OCc3ccccc3)cc12]

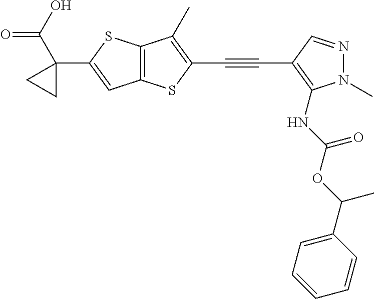 [SMILES: Cc1c(C#Cc2cnn(C)c2NC(=O)OC(C)c2ccccc2)sc2cc(C3(C(=O)O)CC3)sc12]

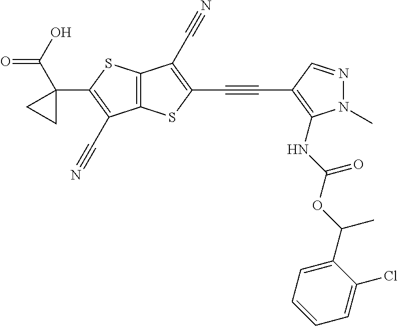 [SMILES: CC(OC(=O)Nc1c(C#Cc2sc3c(C#N)c(C4(C(=O)O)CC4)sc3c2C#N)cnn1C)c1ccccc1Cl]

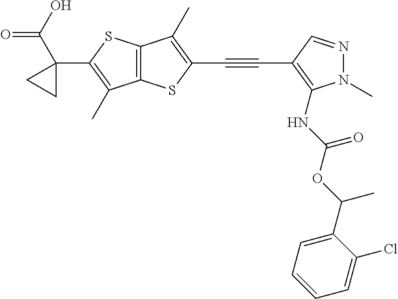 [SMILES: Cc1c(C#Cc2cnn(C)c2NC(=O)OC(C)c2ccccc2Cl)sc2c(C)c(C3(C(=O)O)CC3)sc12]

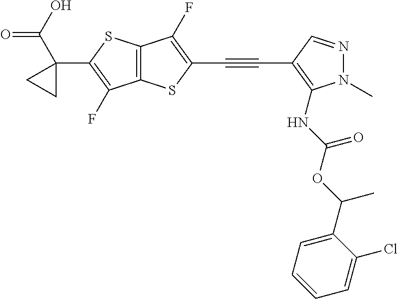 [SMILES: CC(OC(=O)Nc1c(C#Cc2sc3c(F)c(C4(C(=O)O)CC4)sc3c2F)cnn1C)c1ccccc1Cl]

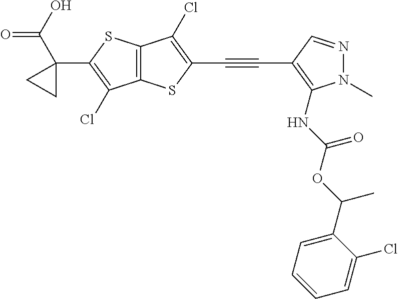 [SMILES: CC(OC(=O)Nc1c(C#Cc2sc3c(Cl)c(C4(C(=O)O)CC4)sc3c2Cl)cnn1C)c1ccccc1Cl]

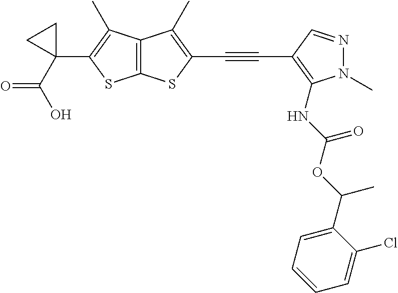 [SMILES: Cc1c(C#Cc2cnn(C)c2NC(=O)OC(C)c2ccccc2Cl)sc2sc(C3(C(=O)O)CC3)c(C)c12]

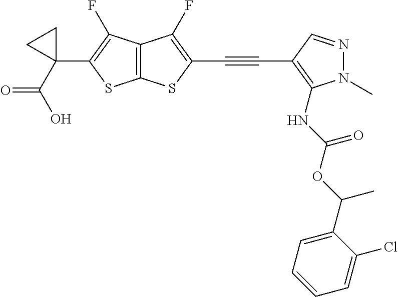 [SMILES: CC(OC(=O)Nc1c(C#Cc2sc3sc(C4(C(=O)O)CC4)c(F)c3c2F)cnn1C)c1ccccc1Cl]

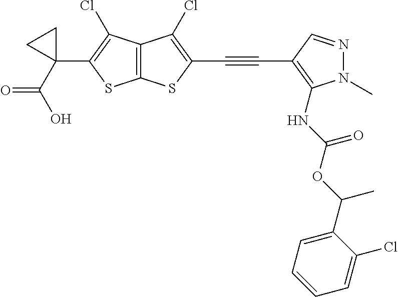 [SMILES: CC(OC(=O)Nc1c(C#Cc2sc3sc(C4(C(=O)O)CC4)c(Cl)c3c2Cl)cnn1C)c1ccccc1Cl]

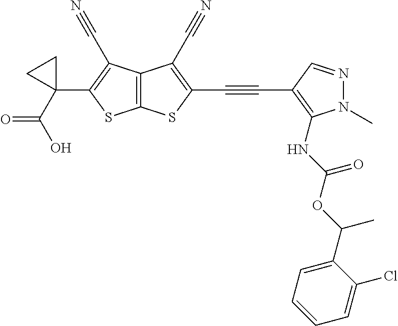 [SMILES: CC(OC(=O)Nc1c(C#Cc2sc3sc(C4(C(=O)O)CC4)c(C#N)c3c2C#N)cnn1C)c1ccccc1Cl]

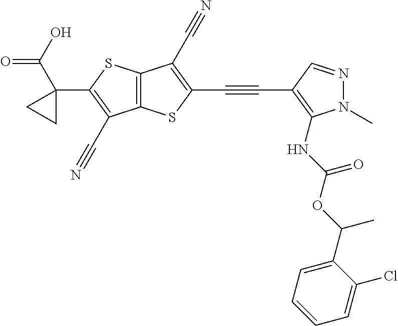 [SMILES: CC(OC(=O)Nc1c(C#Cc2sc3c(C#N)c(C4(C(=O)O)CC4)sc3c2C#N)cnn1C)c1ccccc1Cl]

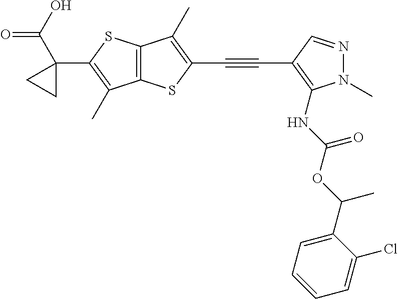 [SMILES: Cc1c(C#Cc2cnn(C)c2NC(=O)OC(C)c2ccccc2Cl)sc2c(C)c(C3(C(=O)O)CC3)sc12]

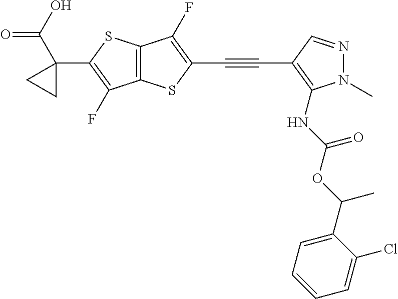 [SMILES: CC(OC(=O)Nc1c(C#Cc2sc3c(F)c(C4(C(=O)O)CC4)sc3c2F)cnn1C)c1ccccc1Cl]

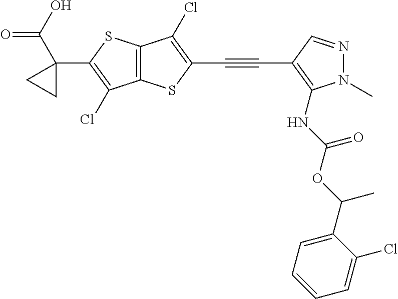 [SMILES: CC(OC(=O)Nc1c(C#Cc2sc3c(Cl)c(C4(C(=O)O)CC4)sc3c2Cl)cnn1C)c1ccccc1Cl]

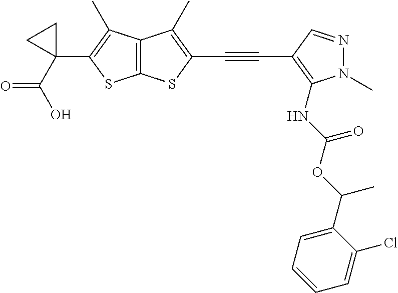 [SMILES: Cc1c(C#Cc2cnn(C)c2NC(=O)OC(C)c2ccccc2Cl)sc2sc(C3(C(=O)O)CC3)c(C)c12]

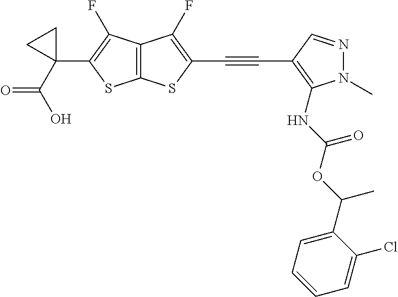 [SMILES: CC(OC(=O)Nc1c(C#Cc2sc3sc(C4(C(=O)O)CC4)c(F)c3c2F)cnn1C)c1ccccc1Cl]

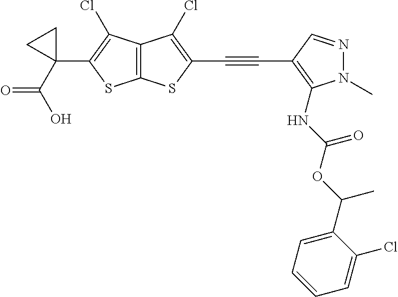 [SMILES: CC(OC(=O)Nc1c(C#Cc2sc3sc(C4(C(=O)O)CC4)c(Cl)c3c2Cl)cnn1C)c1ccccc1Cl]

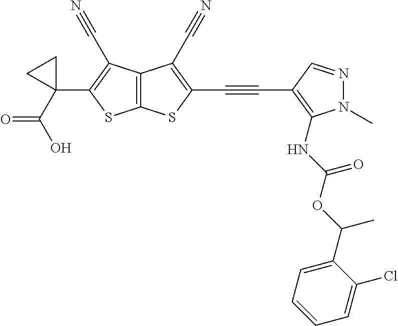 [SMILES: CC(OC(=O)Nc1c(C#Cc2sc3sc(C4(C(=O)O)CC4)c(C#N)c3c2C#N)cnn1C)c1ccccc1Cl]

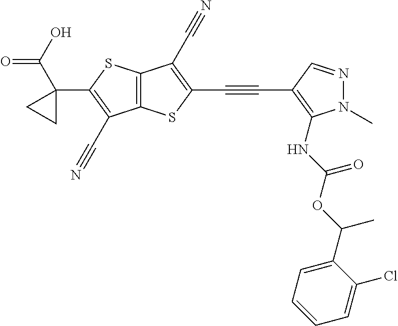 [SMILES: CC(OC(=O)Nc1c(C#Cc2sc3c(C#N)c(C4(C(=O)O)CC4)sc3c2C#N)cnn1C)c1ccccc1Cl]

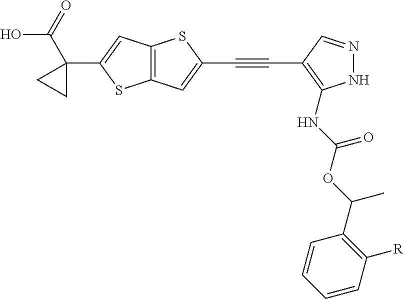

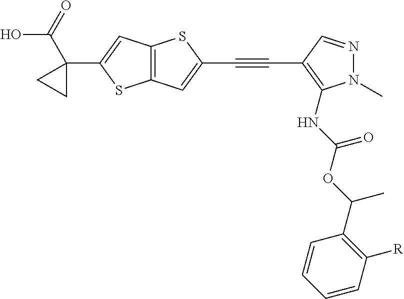

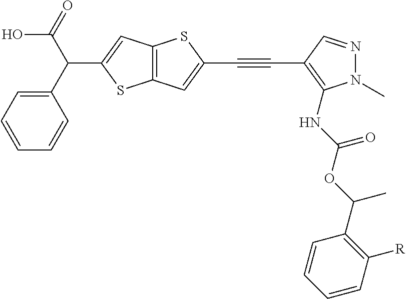

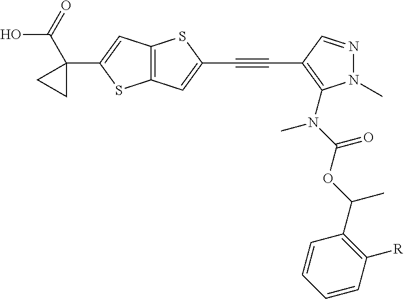

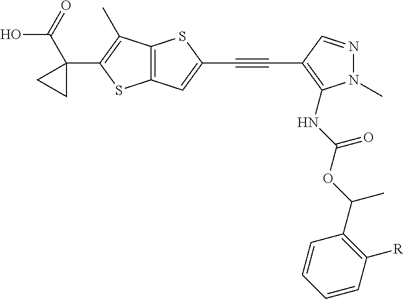

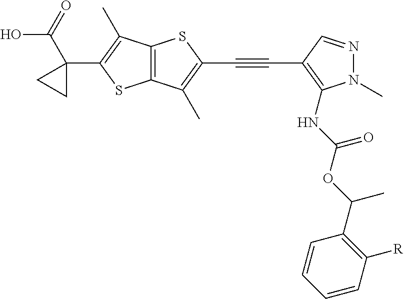

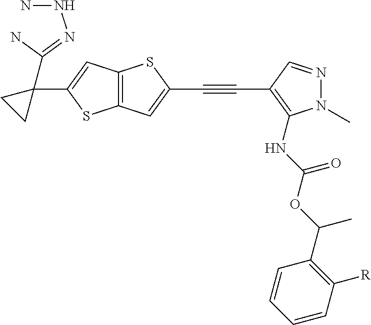

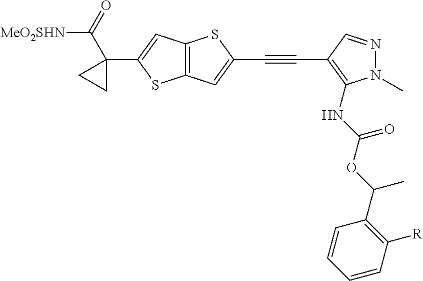

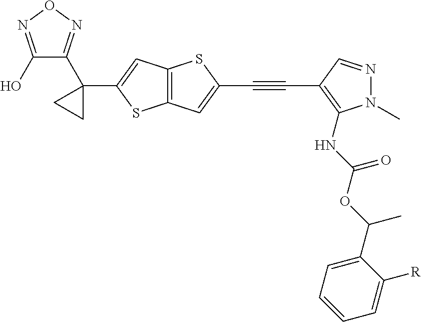

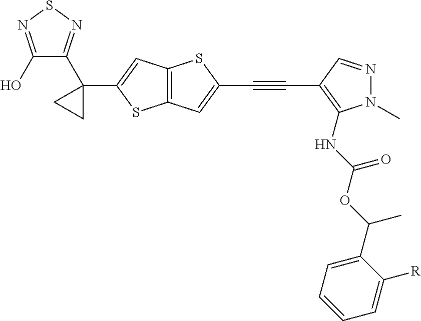

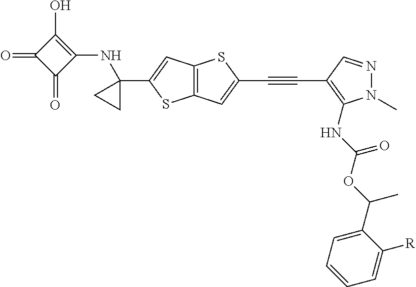

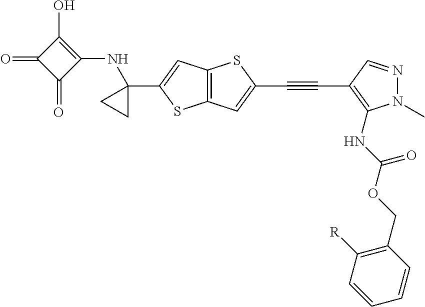

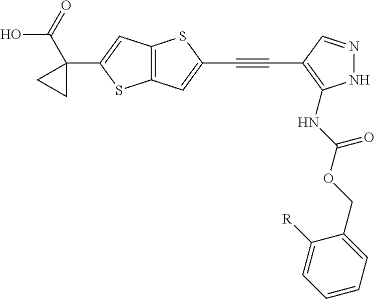

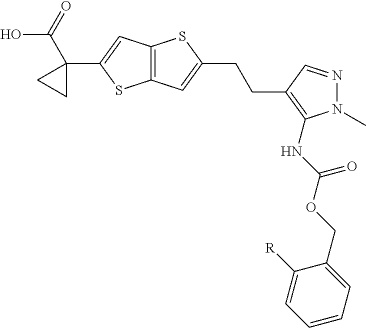

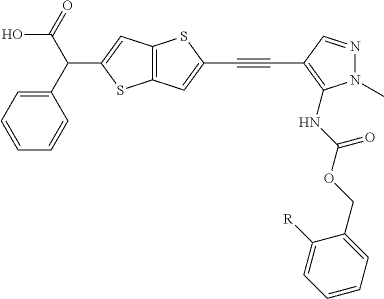

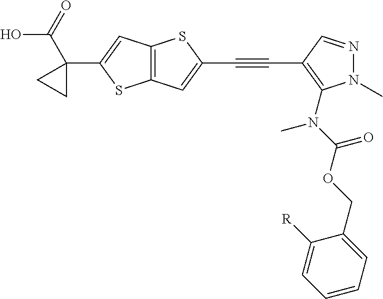

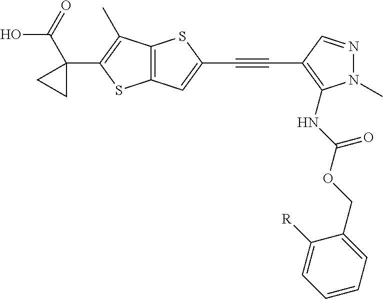

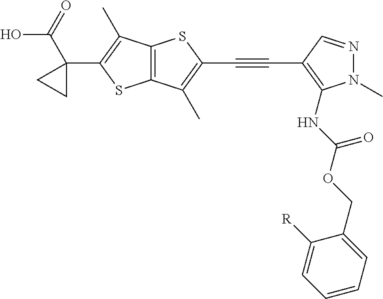

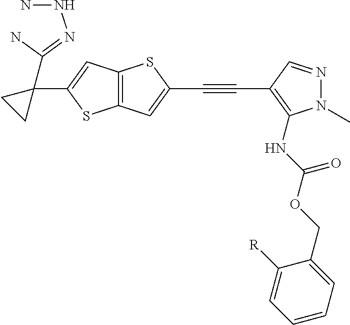

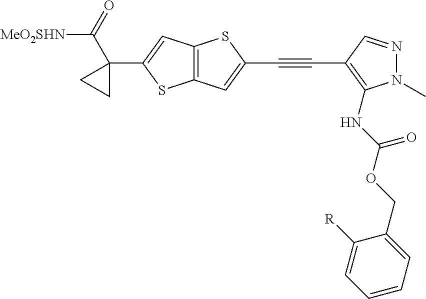

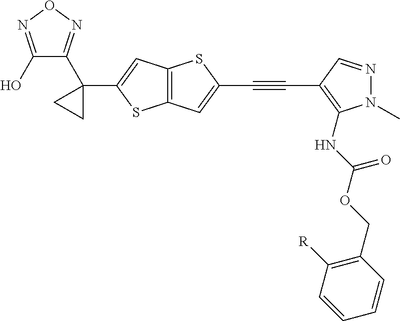

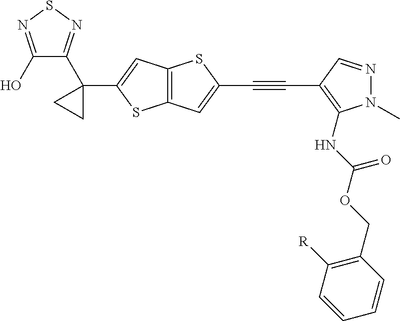

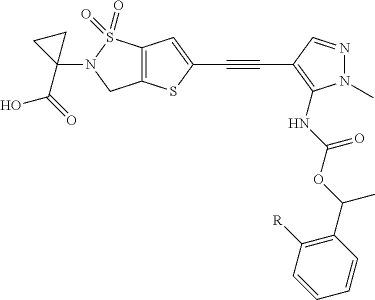

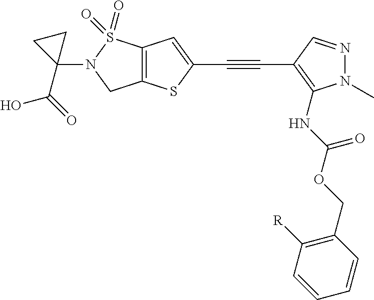

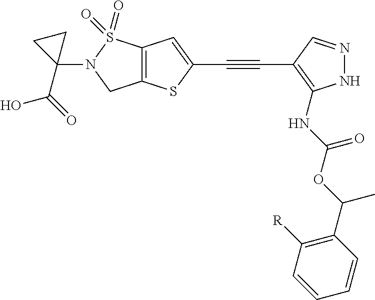

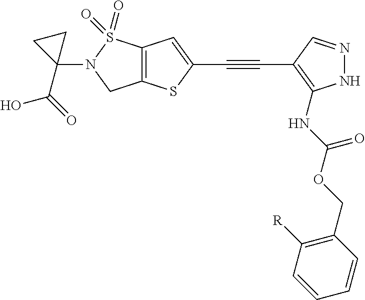

R = H, Me and Cl

In some embodiments, compounds of Formula (VIII) are selected from the following compounds as listed in Table 8.

TABLE 8

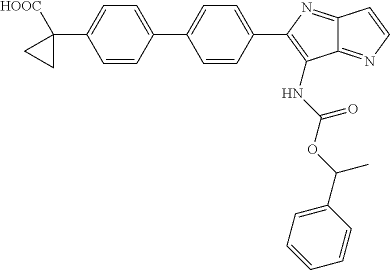 [SMILES: CC(OC(=O)NC1=C(c2ccc(-c3ccc(C4(C(=O)O)CC4)cc3)cc2)N=C2C=CN=C21)c1ccccc1]

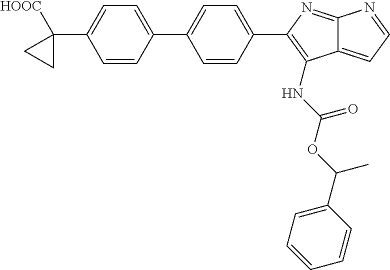 [SMILES: CC(OC(=O)NC1=C(c2ccc(-c3ccc(C4(C(=O)O)CC4)cc3)cc2)N=C2N=CC=C21)c1ccccc1]

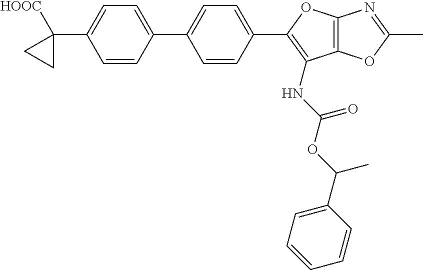 [SMILES: Cc1nc2oc(-c3ccc(-c4ccc(C5(C(=O)O)CC5)cc4)cc3)c(NC(=O)OC(C)c3ccccc3)c2o1]

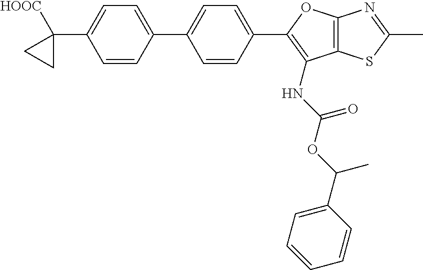 [SMILES: Cc1nc2oc(-c3ccc(-c4ccc(C5(C(=O)O)CC5)cc4)cc3)c(NC(=O)OC(C)c3ccccc3)c2s1]

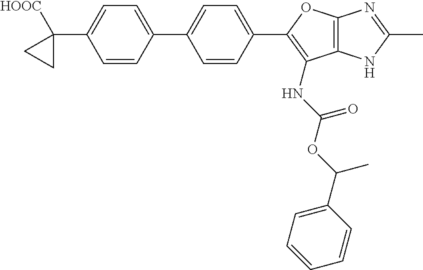 [SMILES: Cc1nc2oc(-c3ccc(-c4ccc(C5(C(=O)O)CC5)cc4)cc3)c(NC(=O)OC(C)c3ccccc3)c2[nH]1]

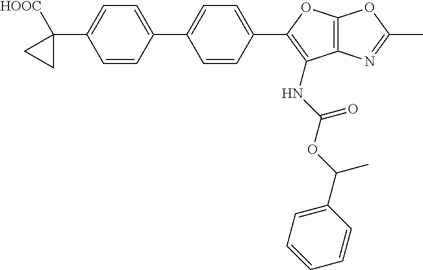 [SMILES: Cc1nc2c(NC(=O)OC(C)c3ccccc3)c(-c3ccc(-c4ccc(C5(C(=O)O)CC5)cc4)cc3)oc2o1]

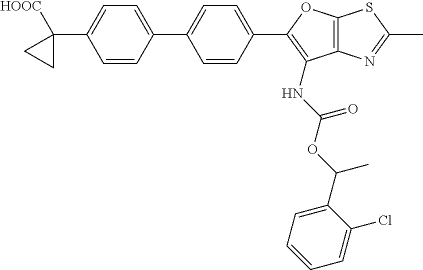 [SMILES: Cc1nc2c(NC(=O)OC(C)c3ccccc3Cl)c(-c3ccc(-c4ccc(C5(C(=O)O)CC5)cc4)cc3)oc2s1]

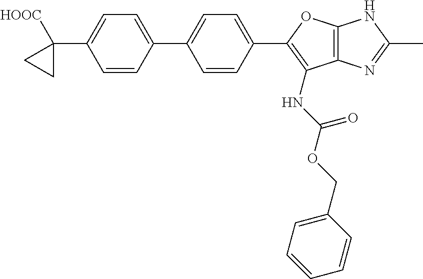 [SMILES: Cc1nc2c(NC(=O)OCc3ccccc3)c(-c3ccc(-c4ccc(C5(C(=O)O)CC5)cc4)cc3)oc2[nH]1]

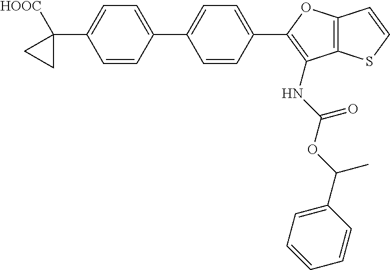 [SMILES: CC(OC(=O)Nc1c(-c2ccc(-c3ccc(C4(C(=O)O)CC4)cc3)cc2)oc2ccsc12)c1ccccc1]

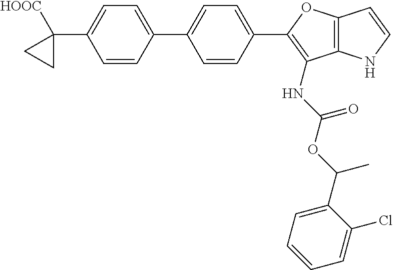 [SMILES: CC(OC(=O)Nc1c(-c2ccc(-c3ccc(C4(C(=O)O)CC4)cc3)cc2)oc2cc[nH]c12)c1ccccc1Cl]

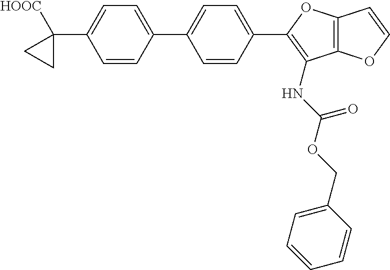 [SMILES: O=C(Nc1c(-c2ccc(-c3ccc(C4(C(=O)O)CC4)cc3)cc2)oc2ccoc12)OCc1ccccc1]

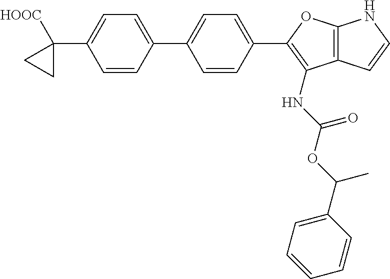 [SMILES: CC(OC(=O)Nc1c(-c2ccc(-c3ccc(C4(C(=O)O)CC4)cc3)cc2)oc2[nH]ccc12)c1ccccc1]

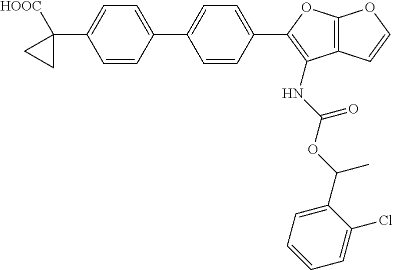 [SMILES: CC(OC(=O)Nc1c(-c2ccc(-c3ccc(C4(C(=O)O)CC4)cc3)cc2)oc2occc12)c1ccccc1Cl]

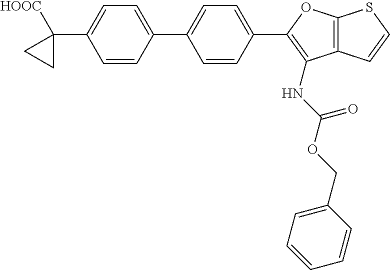 [SMILES: O=C(Nc1c(-c2ccc(-c3ccc(C4(C(=O)O)CC4)cc3)cc2)oc2sccc12)OCc1ccccc1]

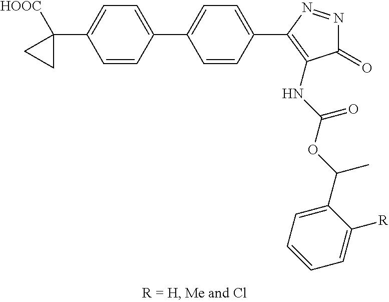

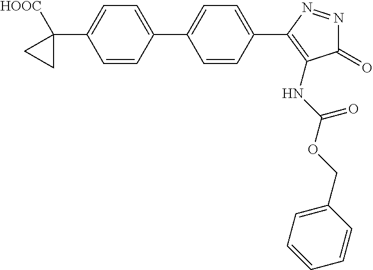 [SMILES: O=C(NC1=C(c2ccc(-c3ccc(C4(C(=O)O)CC4)cc3)cc2)N=NC1=O)OCc1ccccc1]

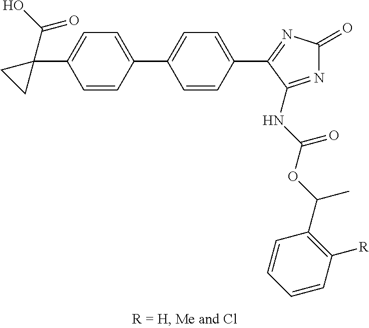

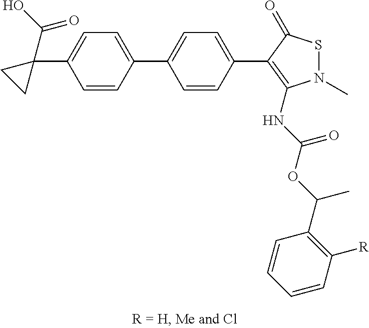

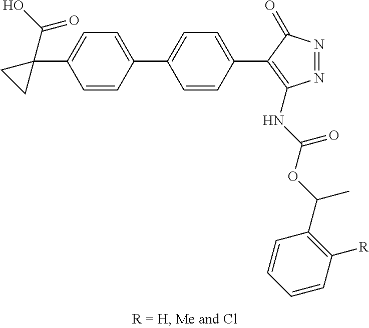

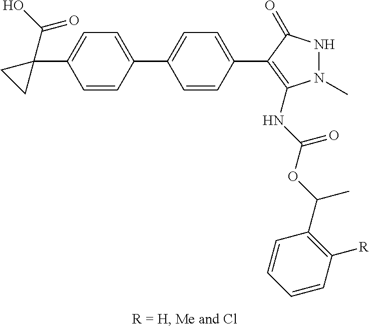

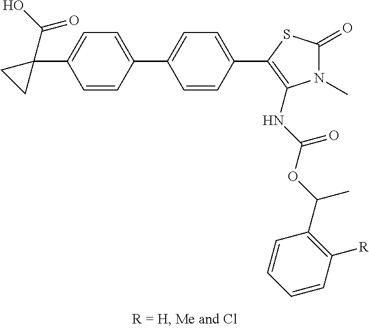

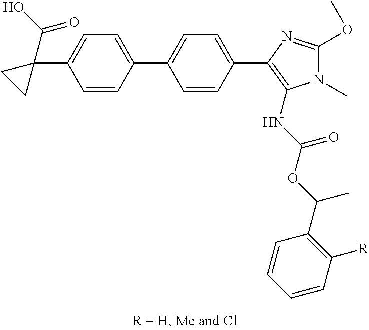

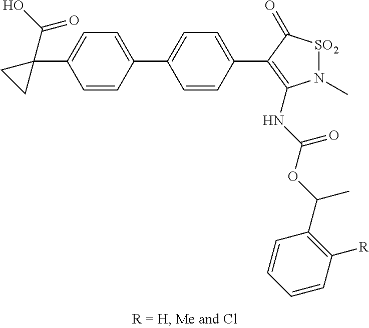

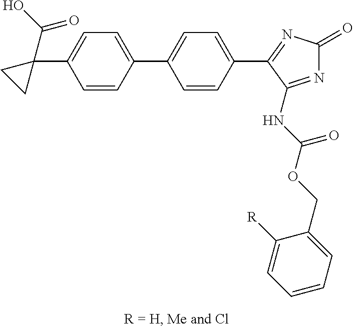

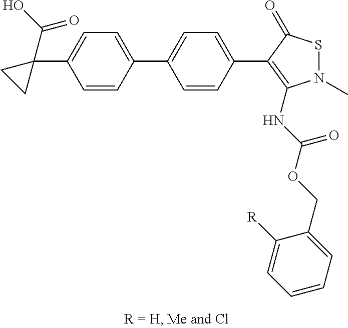

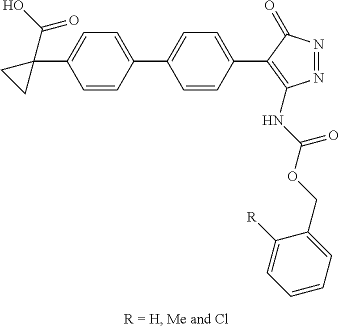

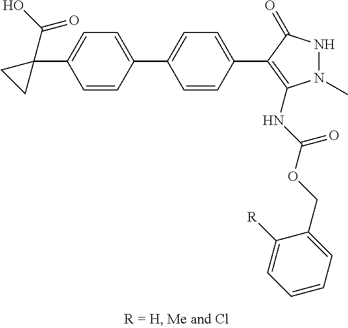

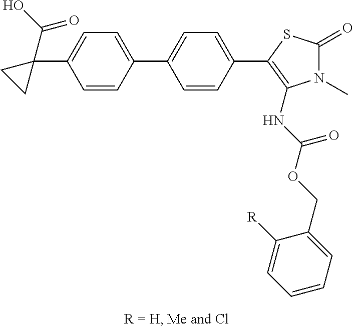

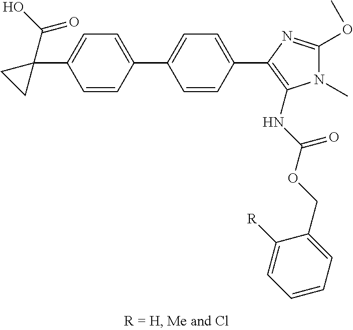

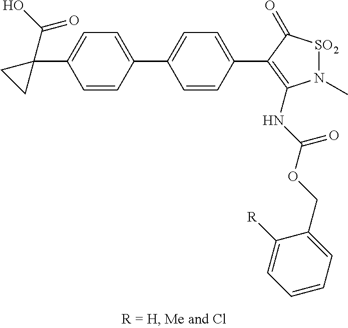

In some embodiments, compounds of Formula (IX) are selected from the following compounds as listed in Table 9.

TABLE 9

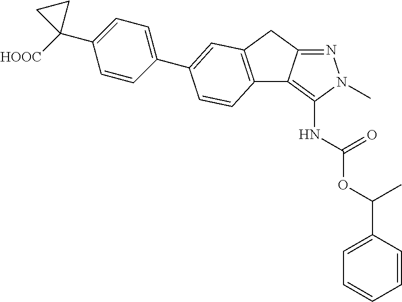 [SMILES: CC(OC(=O)Nc1c2c(nn1C)Cc1cc(-c3ccc(C4(C(=O)O)CC4)cc3)ccc1-2)c1ccccc1]

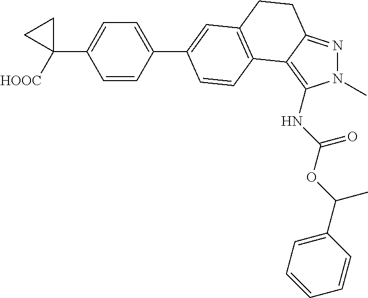 [SMILES: CC(OC(=O)Nc1c2c(nn1C)CCc1cc(-c3ccc(C4(C(=O)O)CC4)cc3)ccc1-2)c1ccccc1]

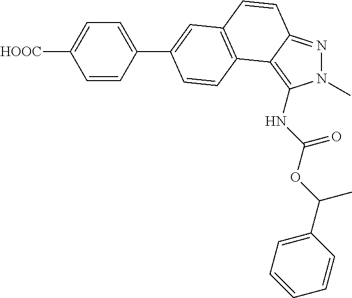 [SMILES: CC(OC(=O)Nc1c2c(ccc3cc(-c4ccc(C(=O)O)cc4)ccc32)nn1C)c1ccccc1]

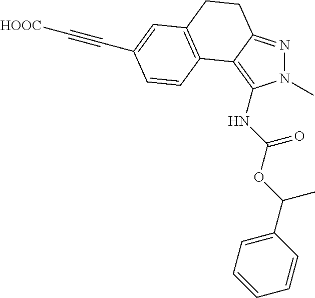 [SMILES: CC(OC(=O)Nc1c2c(nn1C)CCc1cc(C#CC(=O)O)ccc1-2)c1ccccc1]

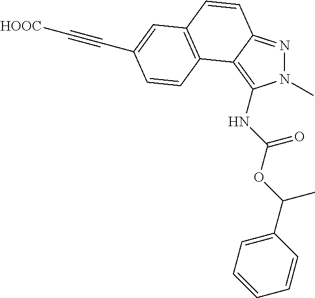 [SMILES: CC(OC(=O)Nc1c2c(ccc3cc(C#CC(=O)O)ccc32)nn1C)c1ccccc1]

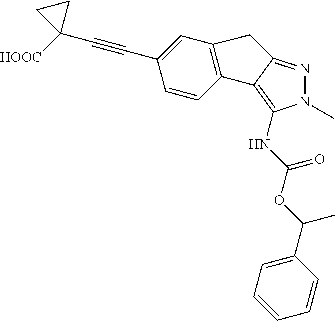 [SMILES: CC(OC(=O)Nc1c2c(nn1C)Cc1cc(C#CC3(C(=O)O)CC3)ccc1-2)c1ccccc1]

In some embodiments, compounds of Formula (XI) are selected from the following compounds as listed in Table 10A.

TABLE 10A

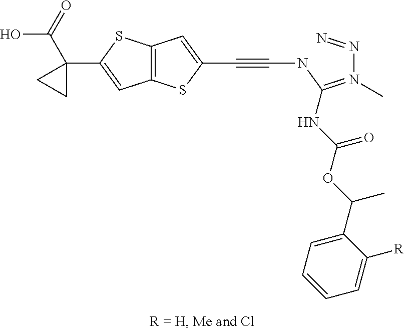

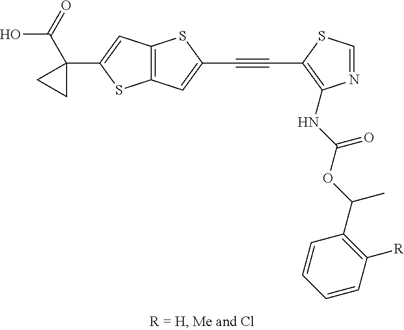

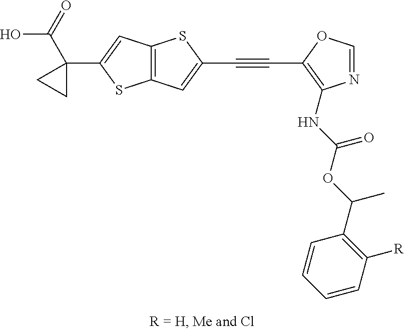

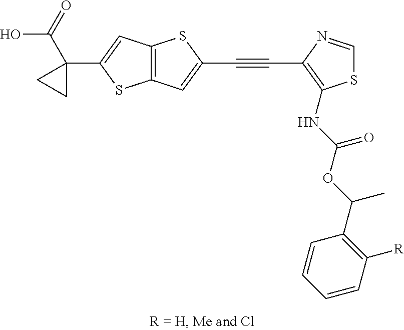

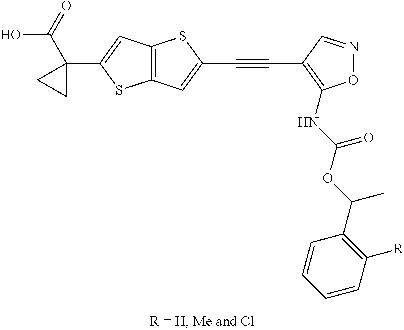

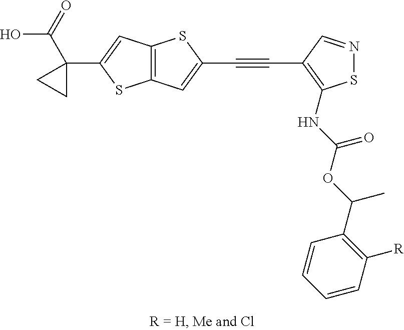

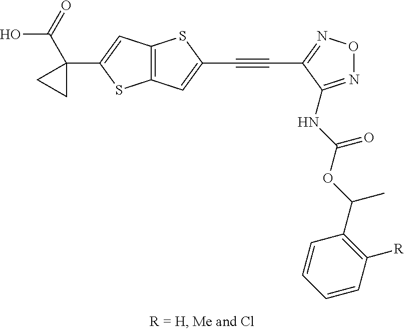

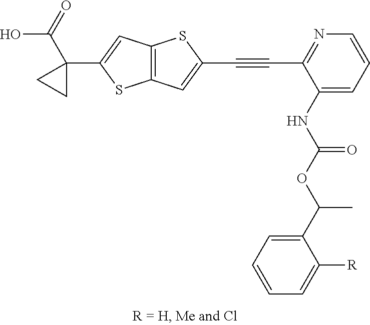

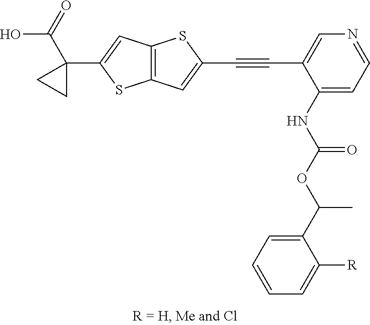

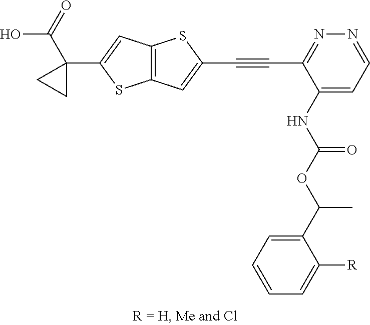

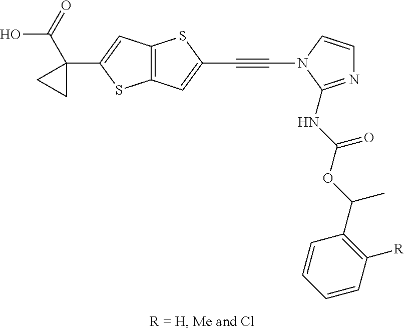

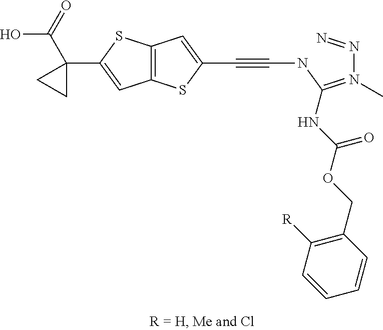

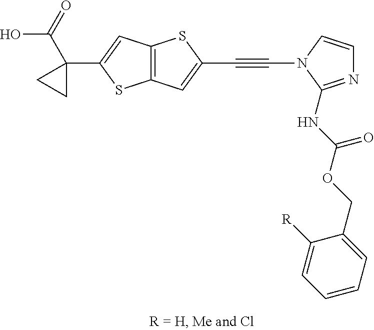

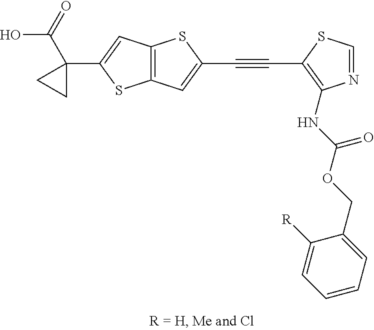

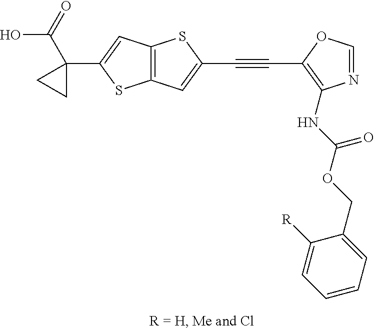

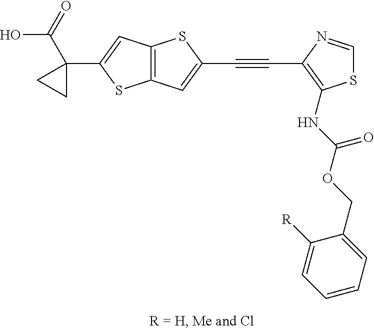

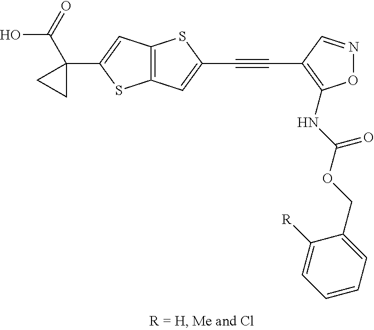

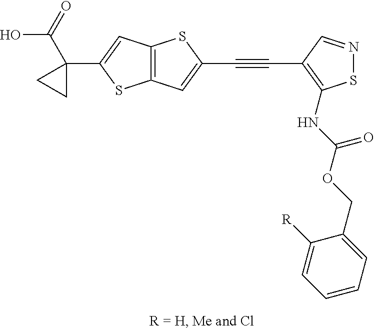

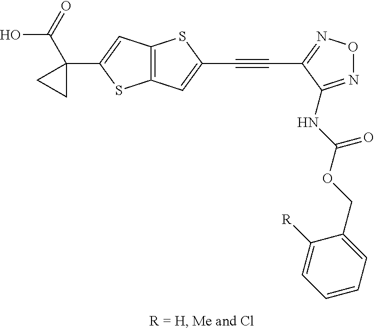

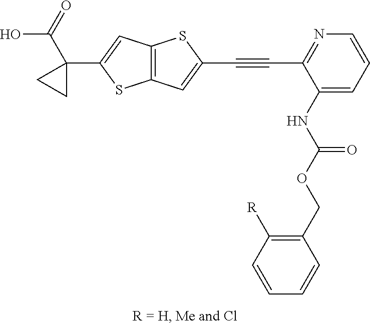

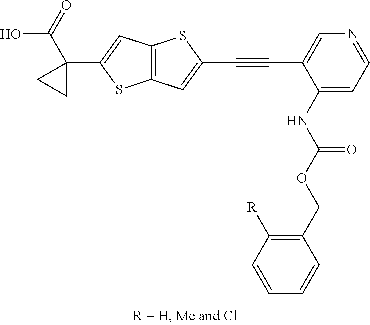

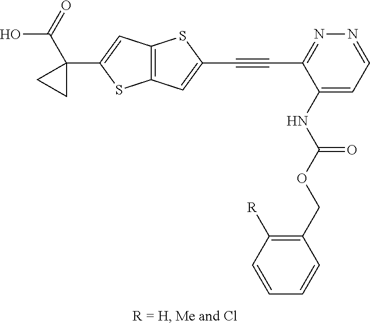

In some embodiments, compounds described herein are selected from the following compounds as listed in Table 10B.

TABLE 10B

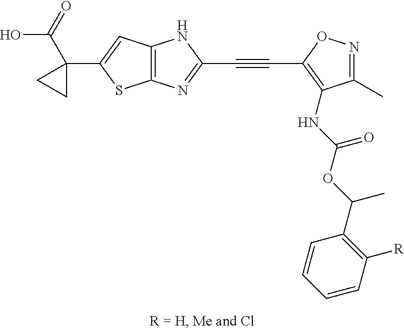

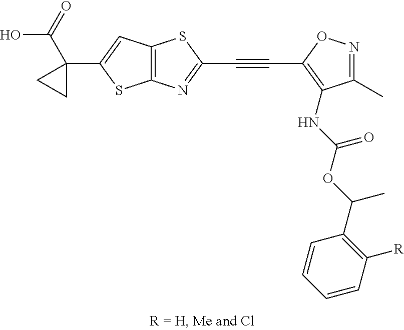

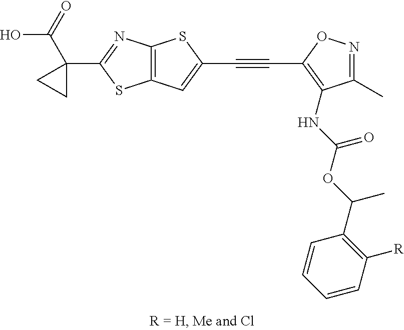

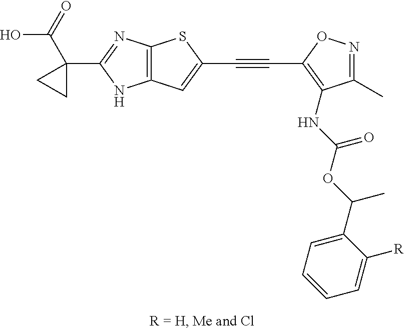

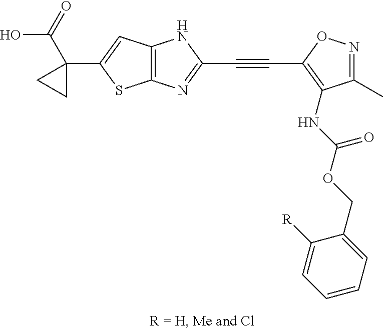

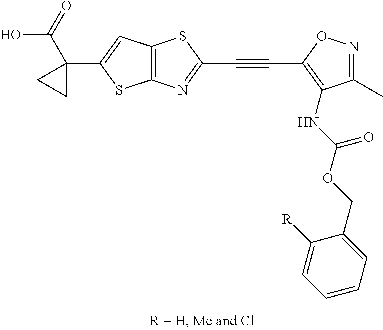

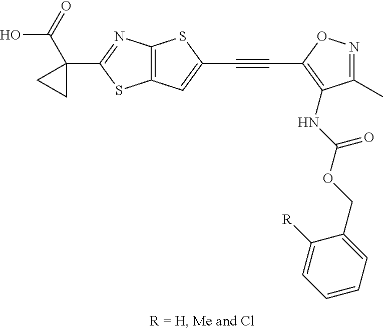

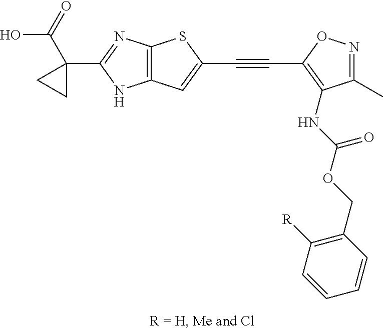

Some embodiments of compounds described herein are selected from the following compounds as listed in Table 11.

TABLE 11

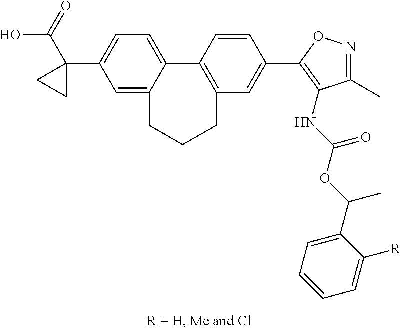

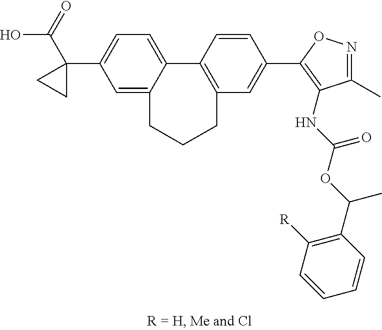

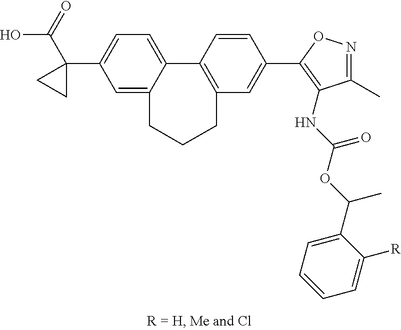

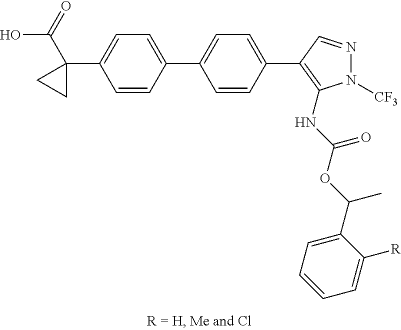

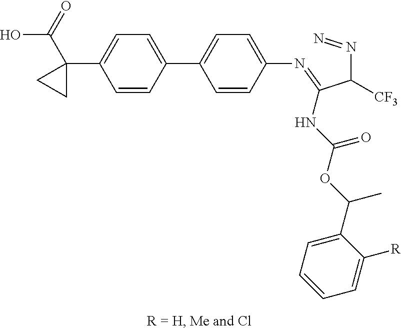

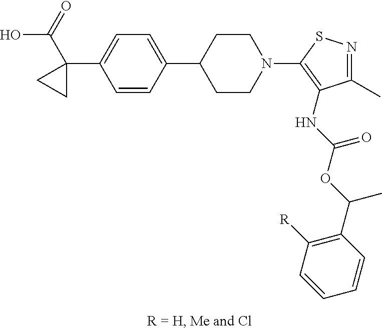

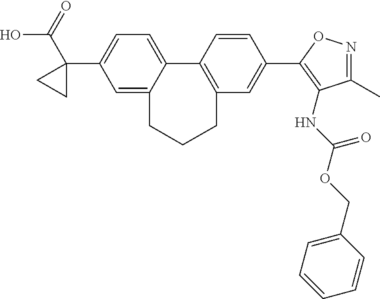 [SMILES: Cc1noc(-c2ccc3c(c2)CCCc2cc(C4(C(=O)O)CC4)ccc2-3)c1NC(=O)OCc1ccccc1]

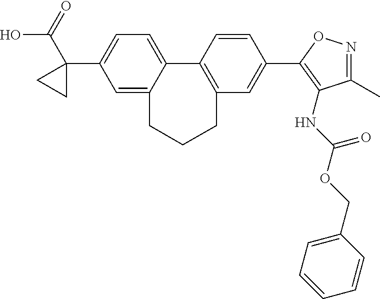 [SMILES: Cc1noc(-c2ccc3c(c2)CCCc2cc(C4(C(=O)O)CC4)ccc2-3)c1NC(=O)OCc1ccccc1]

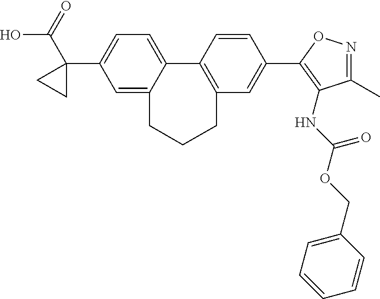 [SMILES: Cc1noc(-c2ccc3c(c2)CCCc2cc(C4(C(=O)O)CC4)ccc2-3)c1NC(=O)OCc1ccccc1]

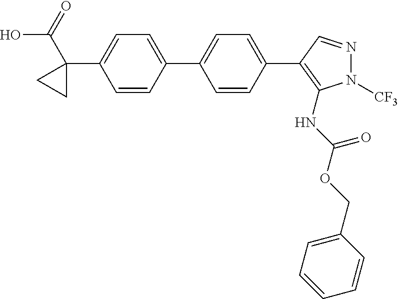 [SMILES: O=C(Nc1c(-c2ccc(-c3ccc(C4(C(=O)O)CC4)cc3)cc2)cnn1C(F)(F)F)OCc1ccccc1]

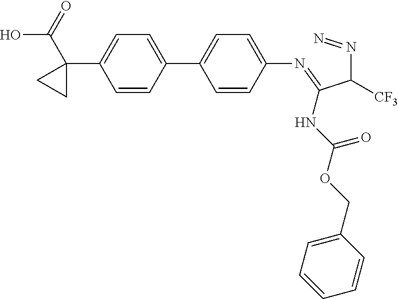 [SMILES: N=NC(/C(=N/c1ccc(-c2ccc(C3(C(=O)O)CC3)cc2)cc1)NC(=O)OCc1ccccc1)C(F)(F)F]

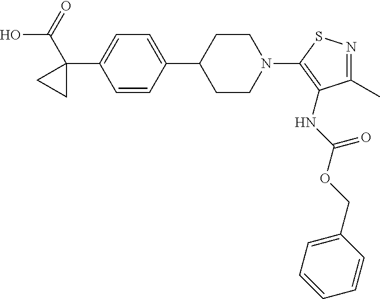 [SMILES: Cc1nsc(N2CCC(c3ccc(C4(C(=O)O)CC4)cc3)CC2)c1NC(=O)OCc1ccccc1]

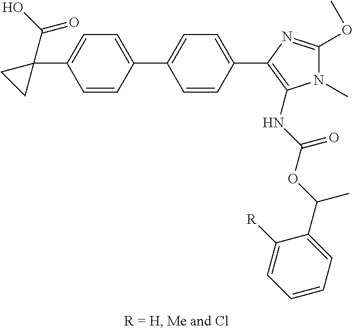

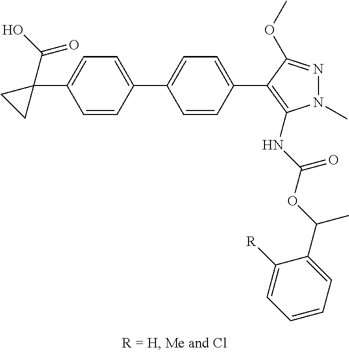

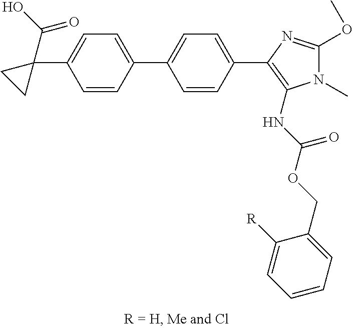

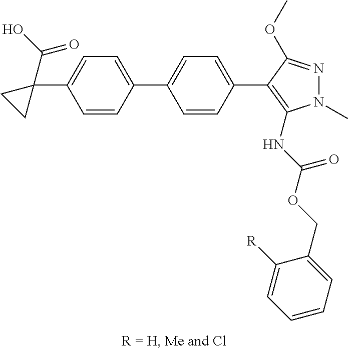

Some embodiments of compounds described herein are selected from the following compounds as listed in Table 12.

TABLE 12

| Compd. | Structure |
|---|---|
| IT001 | 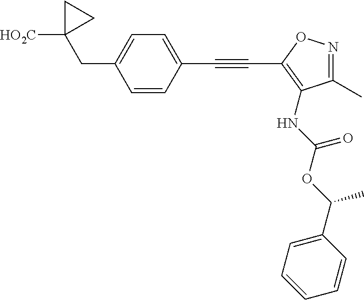 [SMILES: Cc1noc(C#Cc2ccc(CC3(C(=O)O)CC3)cc2)c1NC(=O)O[C@H](C)c1ccccc1] |
| IT002 | 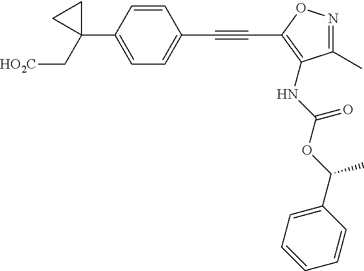 [SMILES: Cc1noc(C#Cc2ccc(C3(CC(=O)O)CC3)cc2)c1NC(=O)O[C@H](C)c1ccccc1] |
| IT003 | 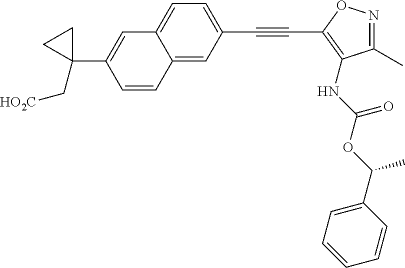 [SMILES: Cc1noc(C#Cc2ccc3cc(C4(CC(=O)O)CC4)ccc3c2)c1NC(=O)O[C@H](C)c1ccccc1] |
| IT004 | 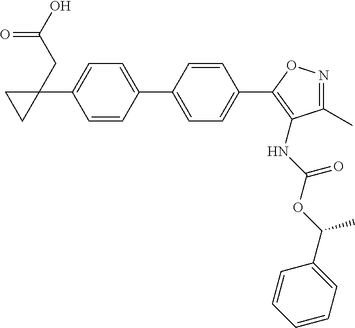 [SMILES: Cc1noc(-c2ccc(-c3ccc(C4(CC(=O)O)CC4)cc3)cc2)c1NC(=O)O[C@H](C)c1ccccc1] |
| IT005 | 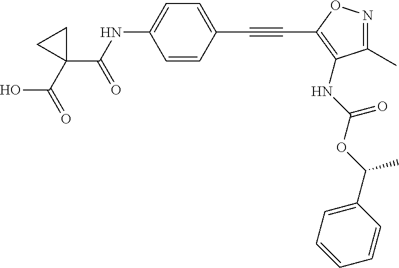 [SMILES: Cc1noc(C#Cc2ccc(NC(=O)C3(C(=O)O)CC3)cc2)c1NC(=O)O[C@H](C)c1ccccc1] |
| IT006 | 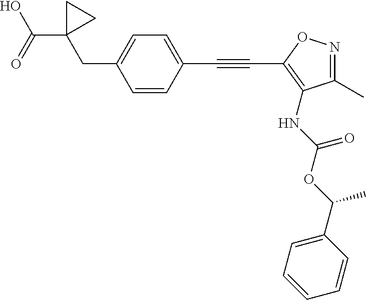 [SMILES: Cc1noc(C#Cc2ccc(CC3(C(=O)O)CC3)cc2)c1NC(=O)O[C@H](C)c1ccccc1] |
| IT007 Isomer 1 | 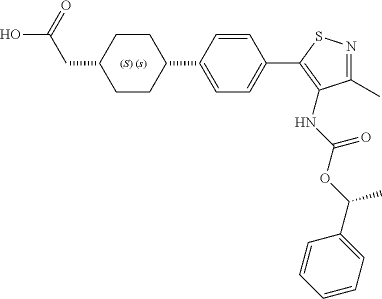 |
| IT008 Isomer 2 | 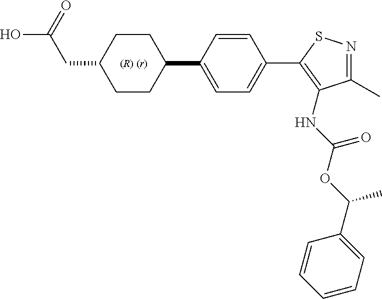 [SMILES: Cc1nsc(-c2ccc([C@H]3CC[C@H](CC(=O)O)CC3)cc2)c1NC(=O)O[C@H](C)c1ccccc1] |
| IT009 | 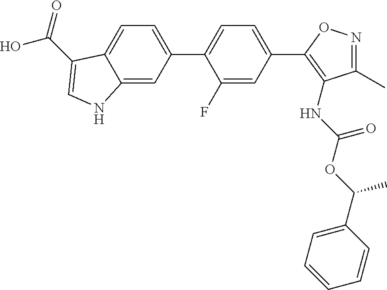 [SMILES: Cc1noc(-c2ccc(-c3ccc4c(C(=O)O)c[nH]c4c3)c(F)c2)c1NC(=O)O[C@H](C)c1ccccc1] |
| IT010 | 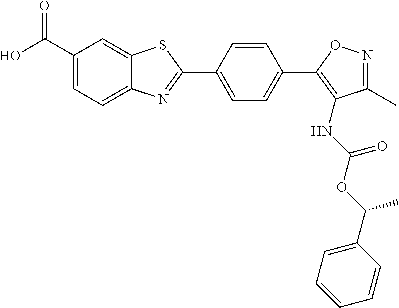 [SMILES: Cc1noc(-c2ccc(-c3nc4ccc(C(=O)O)cc4s3)cc2)c1NC(=O)O[C@H](C)c1ccccc1] |
| IT011 | 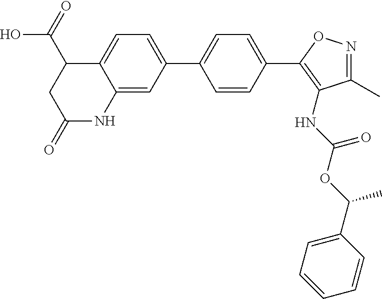 [SMILES: Cc1noc(-c2ccc(-c3ccc4c(c3)NC(=O)CC4C(=O)O)cc2)c1NC(=O)O[C@H](C)c1ccccc1] |
| IT012 | 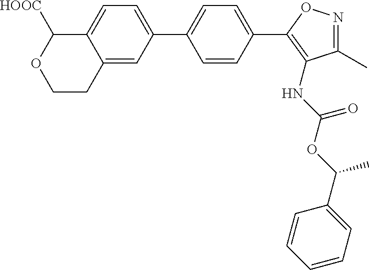 [SMILES: Cc1noc(-c2ccc(-c3ccc4c(c3)CCOC4C(=O)O)cc2)c1NC(=O)O[C@H](C)c1ccccc1] |
| IT013 | 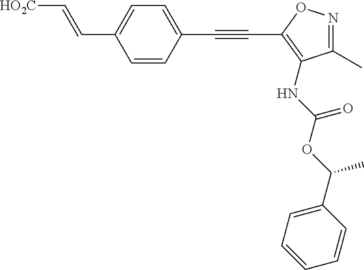 [SMILES: Cc1noc(C#Cc2ccc(/C=C/C(=O)O)cc2)c1NC(=O)O[C@H](C)c1ccccc1] |
| IT014 | 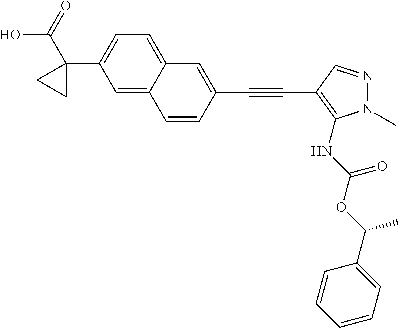 [SMILES: C[C@@H](OC(=O)Nc1c(C#Cc2ccc3cc(C4(C(=O)O)CC4)ccc3c2)cnn1C)c1ccccc1] |
| IT015 | 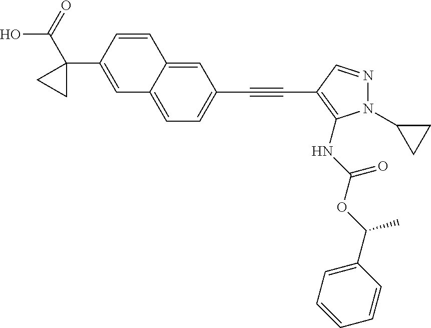 [SMILES: C[C@@H](OC(=O)Nc1c(C#Cc2ccc3cc(C4(C(=O)O)CC4)ccc3c2)cnn1C1CC1)c1ccccc1] |
| IT016 | 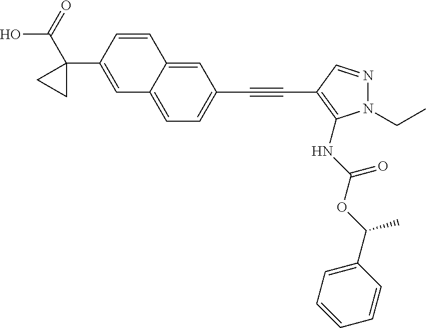 [SMILES: CCn1ncc(C#Cc2ccc3cc(C4(C(=O)O)CC4)ccc3c2)c1NC(=O)O[C@H](C)c1ccccc1] |
| IT017 | 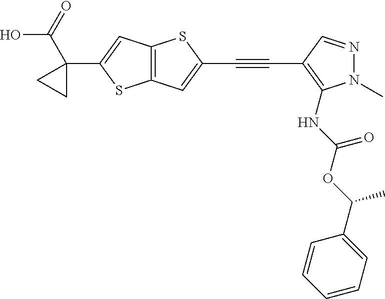 [SMILES: C[C@@H](OC(=O)Nc1c(C#Cc2cc3sc(C4(C(=O)O)CC4)cc3s2)cnn1C)c1ccccc1] |
| IT018 | 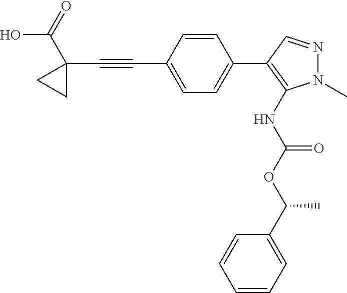 [SMILES: C[C@@H](OC(=O)Nc1c(-c2ccc(C#CC3(C(=O)O)CC3)cc2)cnn1C)c1ccccc1] |
| IT019 | 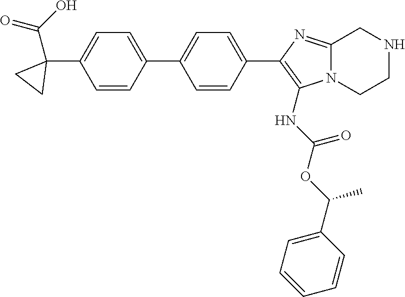 [SMILES: C[C@@H](OC(=O)Nc1c(-c2ccc(-c3ccc(C4(C(=O)O)CC4)cc3)cc2)nc2n1CCNC2)c1ccccc1] |
| IT020 | 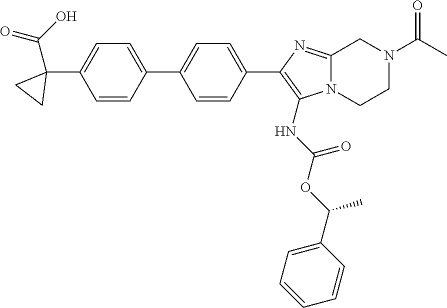 [SMILES: CC(=O)N1CCn2c(nc(-c3ccc(-c4ccc(C5(C(=O)O)CC5)cc4)cc3)c2NC(=O)O[C@H](C)c2ccccc2)C1] |
| IT021 | 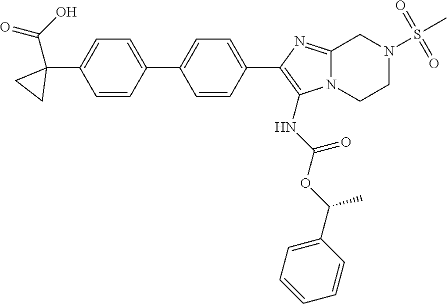 [SMILES: C[C@@H](OC(=O)Nc1c(-c2ccc(-c3ccc(C4(C(=O)O)CC4)cc3)cc2)nc2n1CCN(S(C)(=O)=O)C2)c1ccccc1] |
| IT022 | 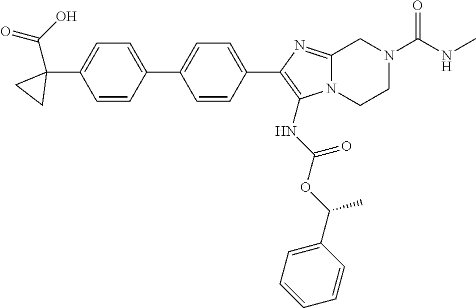 [SMILES: CNC(=O)N1CCn2c(nc(-c3ccc(-c4ccc(C5(C(=O)O)CC5)cc4)cc3)c2NC(=O)O[C@H](C)c2ccccc2)C1] |
| IT023 | 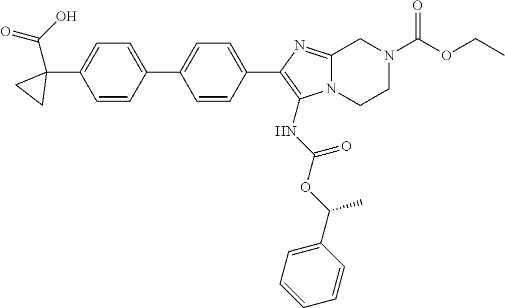 [SMILES: CCOC(=O)N1CCn2c(nc(-c3ccc(-c4ccc(C5(C(=O)O)CC5)cc4)cc3)c2NC(=O)O[C@H](C)c2ccccc2)C1] |
| IT024 | 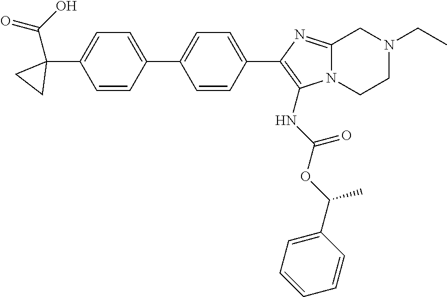 [SMILES: CCN1CCn2c(nc(-c3ccc(-c4ccc(C5(C(=O)O)CC5)cc4)cc3)c2NC(=O)O[C@H](C)c2ccccc2)C1] |
| IT025 | 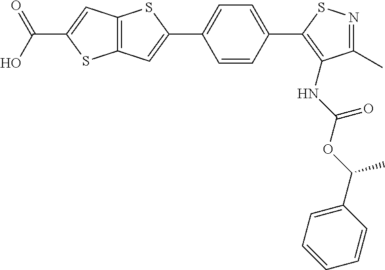 [SMILES: Cc1nsc(-c2ccc(-c3cc4sc(C(=O)O)cc4s3)cc2)c1NC(=O)O[C@H](C)c1ccccc1] |
| IT026 | 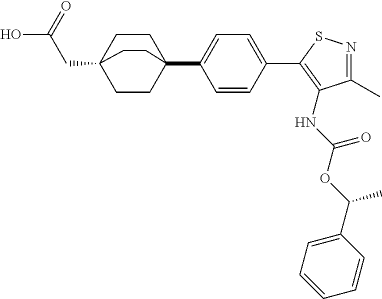 |
| IT027 | 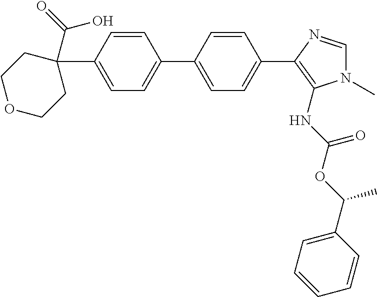 [SMILES: C[C@@H](OC(=O)Nc1c(-c2ccc(-c3ccc(C4(C(=O)O)CCOCC4)cc3)cc2)ncn1C)c1ccccc1] |
| IT028 | 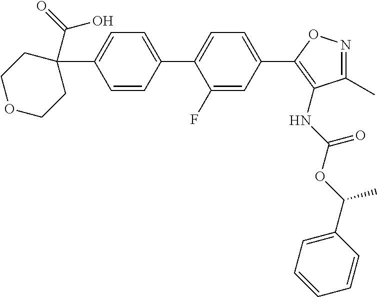 [SMILES: Cc1noc(-c2ccc(-c3ccc(C4(C(=O)O)CCOCC4)cc3)c(F)c2)c1NC(=O)O[C@H](C)c1ccccc1] |
| IT029 | 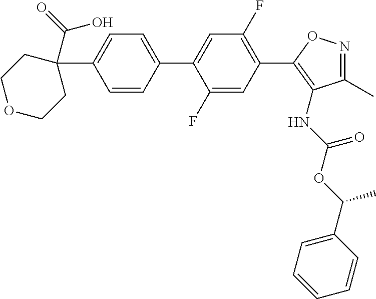 [SMILES: Cc1noc(-c2cc(F)c(-c3ccc(C4(C(=O)O)CCOCC4)cc3)cc2F)c1NC(=O)O[C@H](C)c1ccccc1] |
| IT030 | 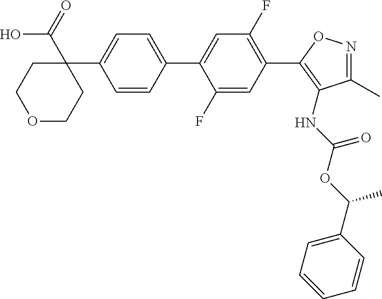 [SMILES: Cc1noc(-c2cc(F)c(-c3ccc(C4(C(=O)O)CCOCC4)cc3)cc2F)c1NC(=O)O[C@H](C)c1ccccc1] |
| IT031 | 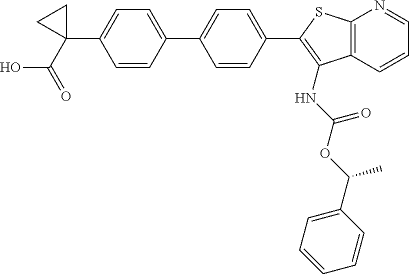 [SMILES: C[C@@H](OC(=O)Nc1c(-c2ccc(-c3ccc(C4(C(=O)O)CC4)cc3)cc2)sc2ncccc12)c1ccccc1] |
| IT032 | 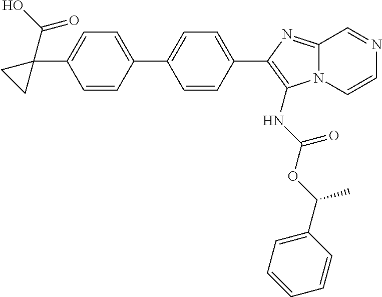 [SMILES: C[C@@H](OC(=O)Nc1c(-c2ccc(-c3ccc(C4(C(=O)O)CC4)cc3)cc2)nc2cnccn12)c1ccccc1] |
| IT033 | 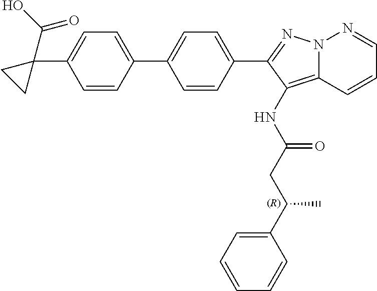 |
| IT034 | 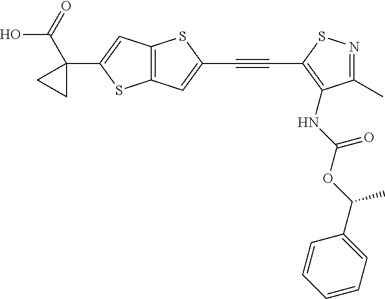 [SMILES: Cc1nsc(C#Cc2cc3sc(C4(C(=O)O)CC4)cc3s2)c1NC(=O)O[C@H](C)c1ccccc1] |
| IT035 | 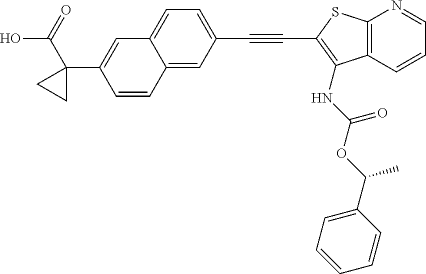 [SMILES: C[C@@H](OC(=O)Nc1c(C#Cc2ccc3cc(C4(C(=O)O)CC4)ccc3c2)sc2ncccc12)c1ccccc1] |
| IT036 | 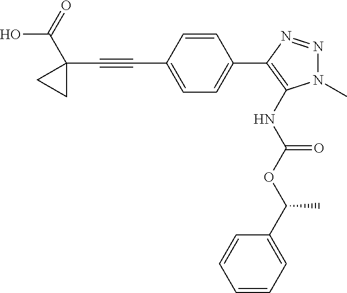 [SMILES: C[C@@H](OC(=O)Nc1c(-c2ccc(C#CC3(C(=O)O)CC3)cc2)nnn1C)c1ccccc1] |
| IT037 | 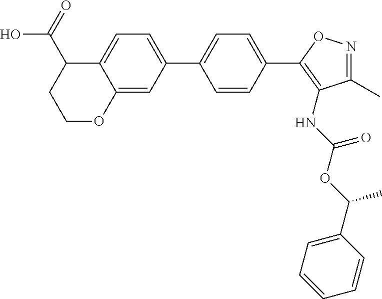 [SMILES: Cc1noc(-c2ccc(-c3ccc4c(c3)OCCC4C(=O)O)cc2)c1NC(=O)O[C@H](C)c1ccccc1] |
| IT038 Isomer 1 | 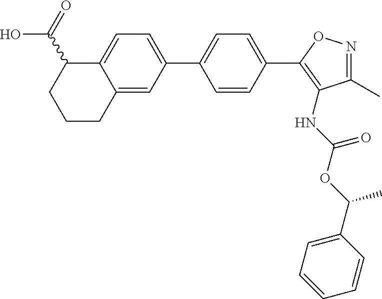 [SMILES: Cc1noc(-c2ccc(-c3ccc4c(c3)CCCC4C(=O)O)cc2)c1NC(=O)O[C@H](C)c1ccccc1] |
| IT039 Isomer 2 | 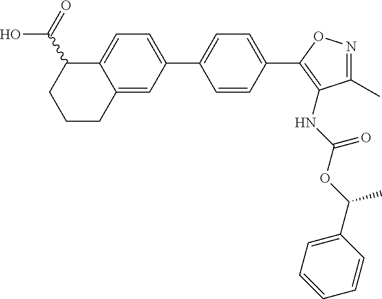 [SMILES: Cc1noc(-c2ccc(-c3ccc4c(c3)CCCC4C(=O)O)cc2)c1NC(=O)O[C@H](C)c1ccccc1] |
| IT040 | 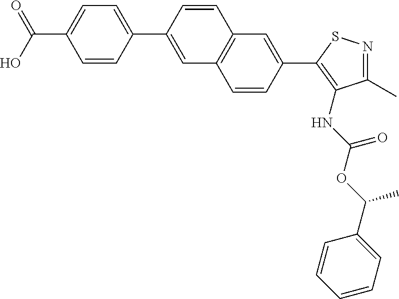 [SMILES: Cc1nsc(-c2ccc3cc(-c4ccc(C(=O)O)cc4)ccc3c2)c1NC(=O)O[C@H](C)c1ccccc1] |
| IT041 | 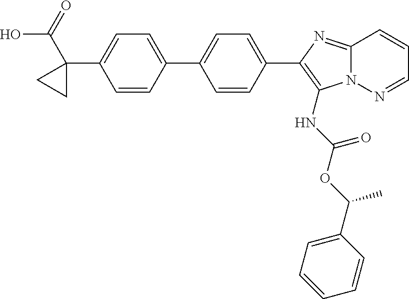 [SMILES: C[C@@H](OC(=O)Nc1c(-c2ccc(-c3ccc(C4(C(=O)O)CC4)cc3)cc2)nc2cccnn12)c1ccccc1] |
| IT042 | 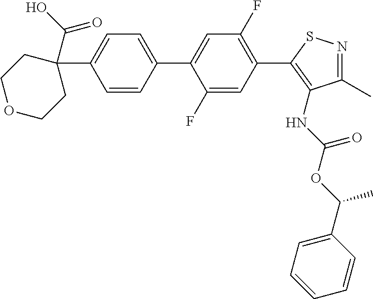 [SMILES: Cc1nsc(-c2cc(F)c(-c3ccc(C4(C(=O)O)CCOCC4)cc3)cc2F)c1NC(=O)O[C@H](C)c1ccccc1] |
| IT043 | 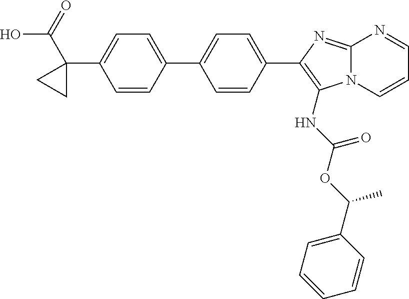 [SMILES: C[C@@H](OC(=O)Nc1c(-c2ccc(-c3ccc(C4(C(=O)O)CC4)cc3)cc2)nc2ncccn12)c1ccccc1] |
| IT044 | 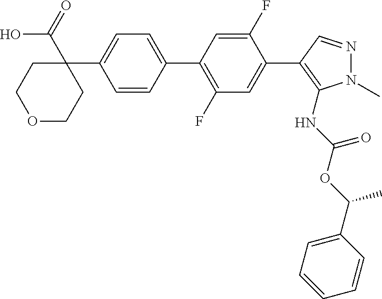 [SMILES: C[C@@H](OC(=O)Nc1c(-c2cc(F)c(-c3ccc(C4(C(=O)O)CCOCC4)cc3)cc2F)cnn1C)c1ccccc1] |
| IT045 | 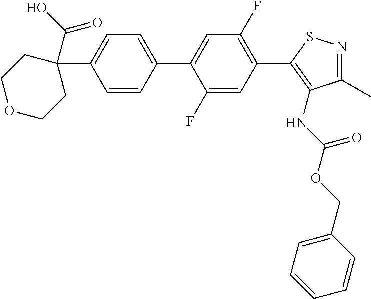 [SMILES: Cc1nsc(-c2cc(F)c(-c3ccc(C4(C(=O)O)CCOCC4)cc3)cc2F)c1NC(=O)OCc1ccccc1] |
| IT046 | 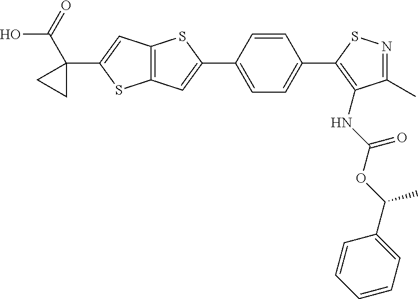 [SMILES: Cc1nsc(-c2ccc(-c3cc4sc(C5(C(=O)O)CC5)cc4s3)cc2)c1NC(=O)O[C@H](C)c1ccccc1] |
| IT047 | 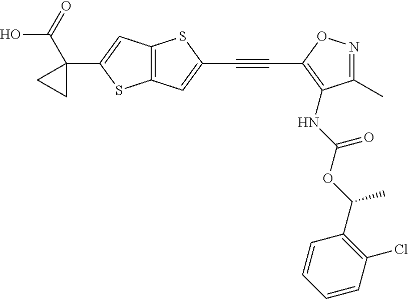 [SMILES: Cc1noc(C#Cc2cc3sc(C4(C(=O)O)CC4)cc3s2)c1NC(=O)O[C@H](C)c1ccccc1Cl] |
| IT048 | 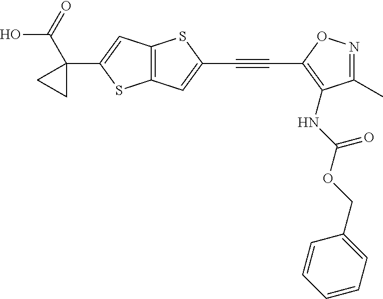 [SMILES: Cc1noc(C#Cc2cc3sc(C4(C(=O)O)CC4)cc3s2)c1NC(=O)OCc1ccccc1] |
| IT049 | 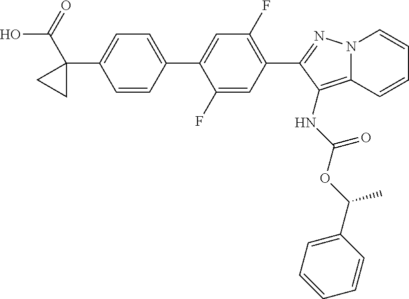 [SMILES: C[C@@H](OC(=O)Nc1c(-c2cc(F)c(-c3ccc(C4(C(=O)O)CC4)cc3)cc2F)nn2ccccc12)c1ccccc1] |
| IT050 | 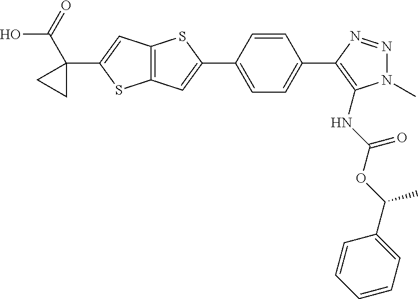 [SMILES: C[C@@H](OC(=O)Nc1c(-c2ccc(-c3cc4sc(C5(C(=O)O)CC5)cc4s3)cc2)nnn1C)c1ccccc1] |
| IT051 | 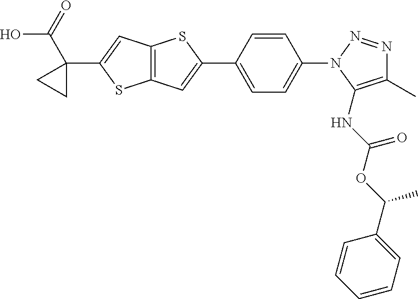 [SMILES: Cc1nnn(-c2ccc(-c3cc4sc(C5(C(=O)O)CC5)cc4s3)cc2)c1NC(=O)O[C@H](C)c1ccccc1] |
| IT052 | 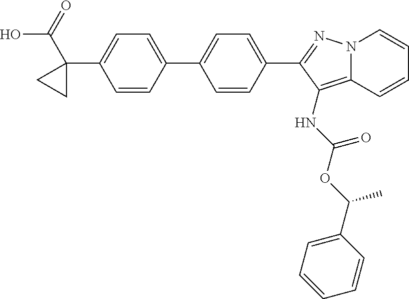 [SMILES: C[C@@H](OC(=O)Nc1c(-c2ccc(-c3ccc(C4(C(=O)O)CC4)cc3)cc2)nn2ccccc12)c1ccccc1] |
| IT053 | 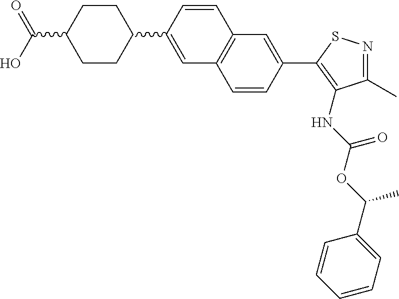 [SMILES: Cc1nsc(-c2ccc3cc(C4CCC(C(=O)O)CC4)ccc3c2)c1NC(=O)O[C@H](C)c1ccccc1] |
| IT054 | 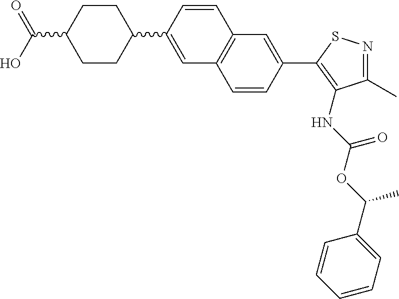 [SMILES: Cc1nsc(-c2ccc3cc(C4CCC(C(=O)O)CC4)ccc3c2)c1NC(=O)O[C@H](C)c1ccccc1] |
| IT055 | 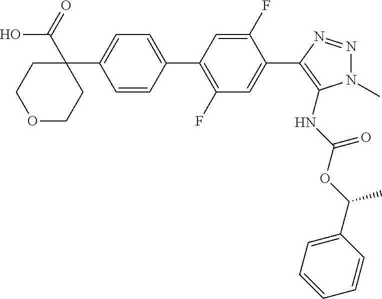 [SMILES: C[C@@H](OC(=O)Nc1c(-c2cc(F)c(-c3ccc(C4(C(=O)O)CCOCC4)cc3)cc2F)nnn1C)c1ccccc1] |
| IT056 | 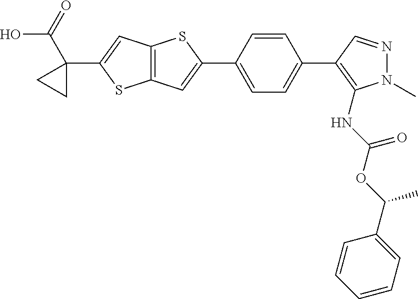 [SMILES: C[C@@H](OC(=O)Nc1c(-c2ccc(-c3cc4sc(C5(C(=O)O)CC5)cc4s3)cc2)cnn1C)c1ccccc1] |
| IT057 Isomer 1 | 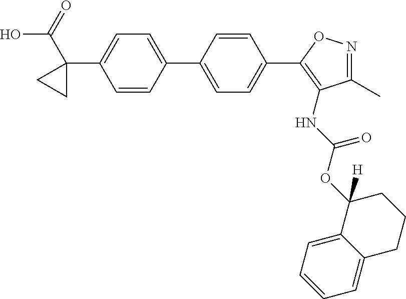 [SMILES: Cc1noc(-c2ccc(-c3ccc(C4(C(=O)O)CC4)cc3)cc2)c1NC(=O)O[C@@H]1CCCc2ccccc21] |
| IT058 Isomer 2 | 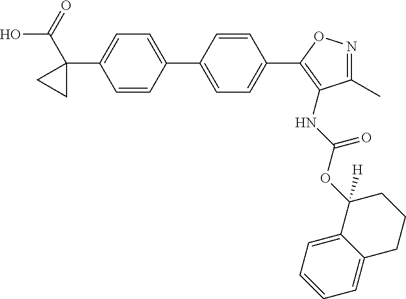 [SMILES: Cc1noc(-c2ccc(-c3ccc(C4(C(=O)O)CC4)cc3)cc2)c1NC(=O)O[C@H]1CCCc2ccccc21] |
| IT059 Isomer 1 | 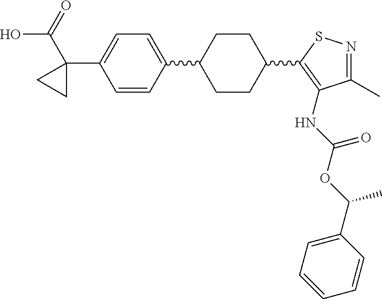 [SMILES: Cc1nsc(C2CCC(c3ccc(C4(C(=O)O)CC4)cc3)CC2)c1NC(=O)O[C@H](C)c1ccccc1] |
| IT060 Isomer 2 | 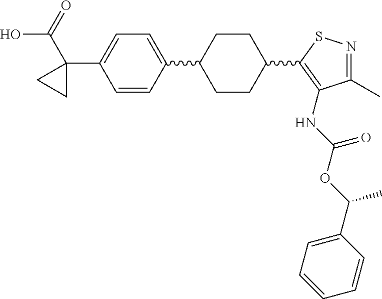 [SMILES: Cc1nsc(C2CCC(c3ccc(C4(C(=O)O)CC4)cc3)CC2)c1NC(=O)O[C@H](C)c1ccccc1] |
| IT061 | 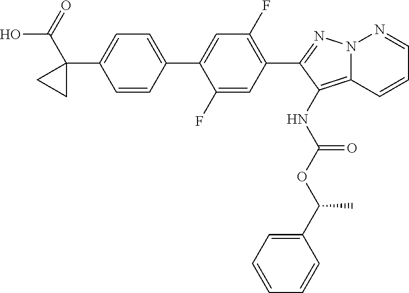 [SMILES: C[C@@H](OC(=O)Nc1c(-c2cc(F)c(-c3ccc(C4(C(=O)O)CC4)cc3)cc2F)nn2ncccc12)c1ccccc1] |
| IT062 Isomer 1 | 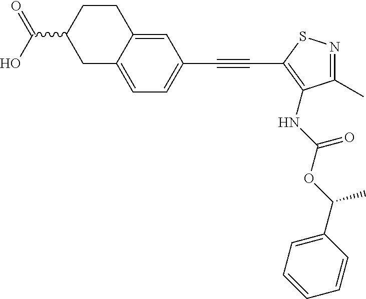 [SMILES: Cc1nsc(C#Cc2ccc3c(c2)CCC(C(=O)O)C3)c1NC(=O)O[C@H](C)c1ccccc1] |
| IT063 Isomer 2 | 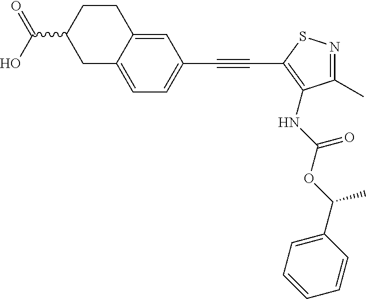 [SMILES: Cc1nsc(C#Cc2ccc3c(c2)CCC(C(=O)O)C3)c1NC(=O)O[C@H](C)c1ccccc1] |
| IT064 | 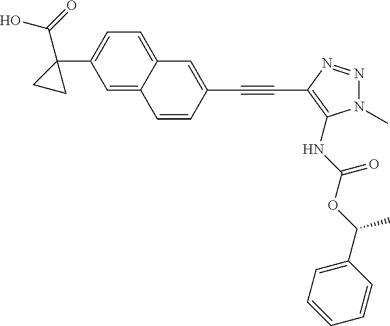 [SMILES: C[C@@H](OC(=O)Nc1c(C#Cc2ccc3cc(C4(C(=O)O)CC4)ccc3c2)nnn1C)c1ccccc1] |
| IT065 | 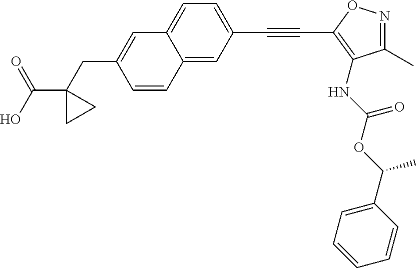 [SMILES: Cc1noc(C#Cc2ccc3cc(CC4(C(=O)O)CC4)ccc3c2)c1NC(=O)O[C@H](C)c1ccccc1] |
| IT066 | 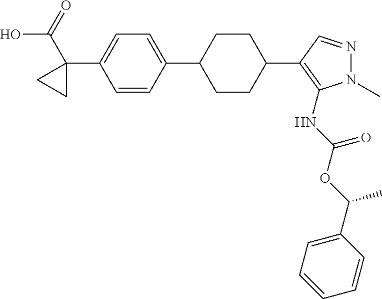 [SMILES: C[C@@H](OC(=O)Nc1c(C2CCC(c3ccc(C4(C(=O)O)CC4)cc3)CC2)cnn1C)c1ccccc1] |
| IT067 | 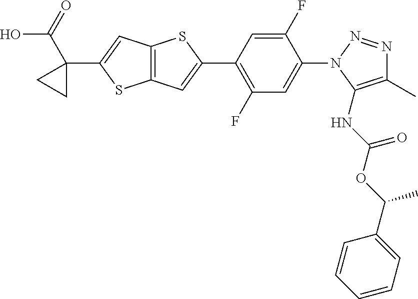 [SMILES: Cc1nnn(-c2cc(F)c(-c3cc4sc(C5(C(=O)O)CC5)cc4s3)cc2F)c1NC(=O)O[C@H](C)c1ccccc1] |
| IT068 | 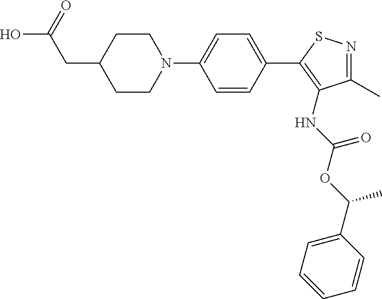 [SMILES: Cc1nsc(-c2ccc(N3CCC(CC(=O)O)CC3)cc2)c1NC(=O)O[C@H](C)c1ccccc1] |
| IT069 | 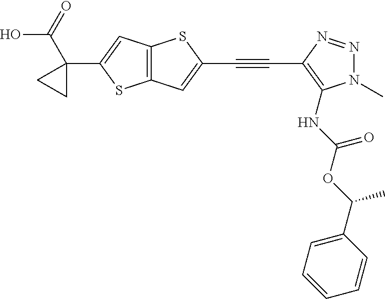 [SMILES: C[C@@H](OC(=O)Nc1c(C#Cc2cc3sc(C4(C(=O)O)CC4)cc3s2)nnn1C)c1ccccc1] |
| IT070 | 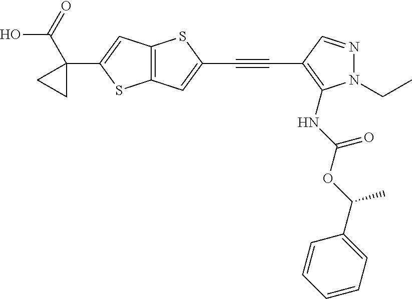 [SMILES: CCn1ncc(C#Cc2cc3sc(C4(C(=O)O)CC4)cc3s2)c1NC(=O)O[C@H](C)c1ccccc1] |
| IT071 | 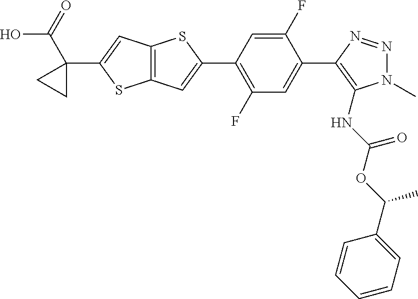 [SMILES: C[C@@H](OC(=O)Nc1c(-c2cc(F)c(-c3cc4sc(C5(C(=O)O)CC5)cc4s3)cc2F)nnn1C)c1ccccc1] |
| IT072 | 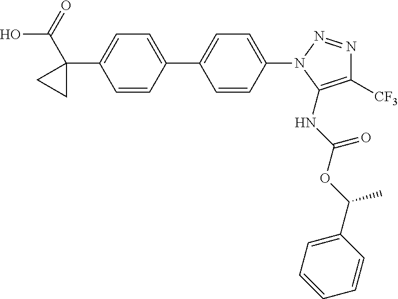 [SMILES: C[C@@H](OC(=O)Nc1c(C(F)(F)F)nnn1-c1ccc(-c2ccc(C3(C(=O)O)CC3)cc2)cc1)c1ccccc1] |
| IT073 | 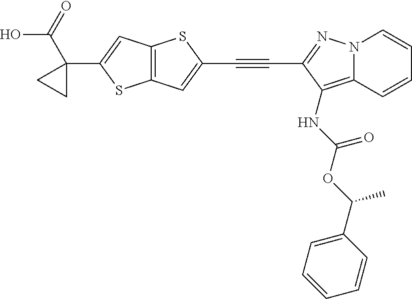 [SMILES: C[C@@H](OC(=O)Nc1c(C#Cc2cc3sc(C4(C(=O)O)CC4)cc3s2)nn2ccccc12)c1ccccc1] |
| IT074 | 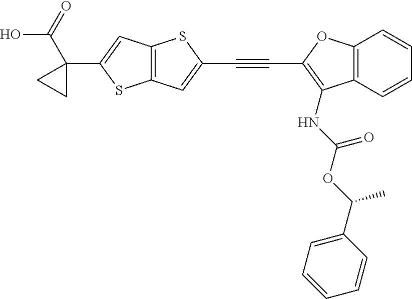 [SMILES: C[C@@H](OC(=O)Nc1c(C#Cc2cc3sc(C4(C(=O)O)CC4)cc3s2)oc2ccccc12)c1ccccc1] |
| IT075 | 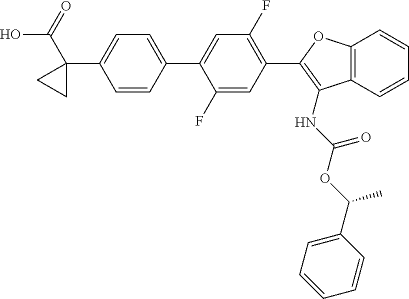 [SMILES: C[C@@H](OC(=O)Nc1c(-c2cc(F)c(-c3ccc(C4(C(=O)O)CC4)cc3)cc2F)oc2ccccc12)c1ccccc1] |
| IT076 Isomer 1 | 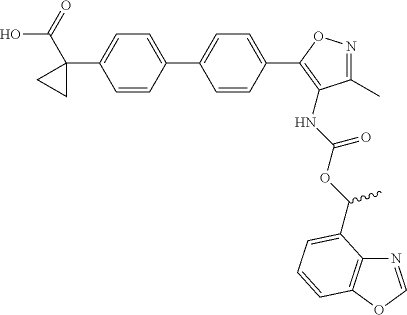 [SMILES: Cc1noc(-c2ccc(-c3ccc(C4(C(=O)O)CC4)cc3)cc2)c1NC(=O)OC(C)c1cccc2ocnc12] |
| IT077 Isomer 2 | 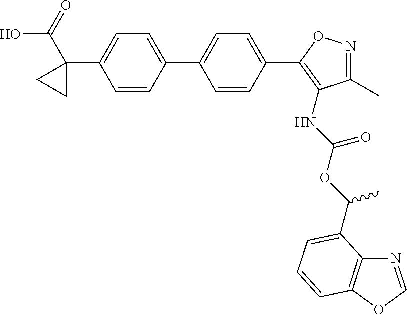 [SMILES: Cc1noc(-c2ccc(-c3ccc(C4(C(=O)O)CC4)cc3)cc2)c1NC(=O)OC(C)c1cccc2ocnc12] |
| IT078 Isomer 1 | 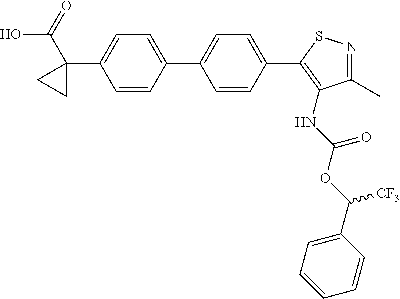 [SMILES: Cc1nsc(-c2ccc(-c3ccc(C4(C(=O)O)CC4)cc3)cc2)c1NC(=O)OC(c1ccccc1)C(F)(F)F] |
| IT079 Isomer 2 | 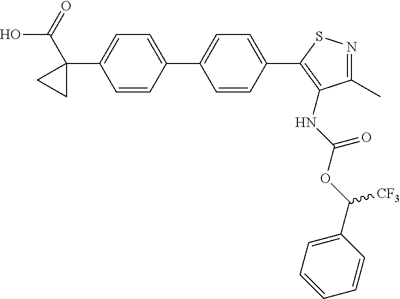 [SMILES: Cc1nsc(-c2ccc(-c3ccc(C4(C(=O)O)CC4)cc3)cc2)c1NC(=O)OC(c1ccccc1)C(F)(F)F] |
| IT080 | 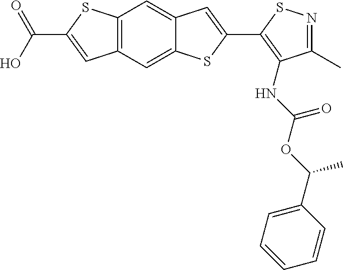 [SMILES: Cc1nsc(-c2cc3cc4sc(C(=O)O)cc4cc3s2)c1NC(=O)O[C@H](C)c1ccccc1] |
| IT081 | 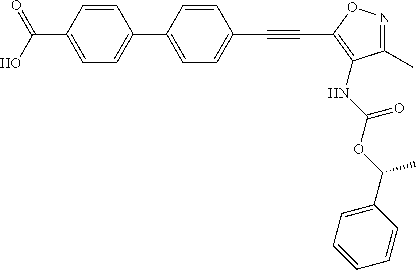 [SMILES: Cc1noc(C#Cc2ccc(-c3ccc(C(=O)O)cc3)cc2)c1NC(=O)O[C@H](C)c1ccccc1] |
| IT082 | 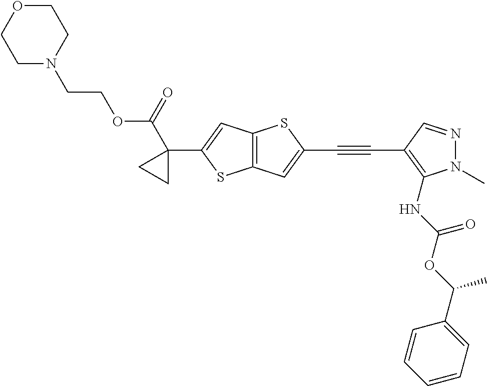 [SMILES: C[C@@H](OC(=O)Nc1c(C#Cc2cc3sc(C4(C(=O)OCCN5CCOCC5)CC4)cc3s2)cnn1C)c1ccccc1] |
| IT083 | 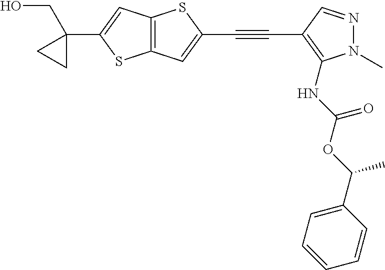 [SMILES: C[C@@H](OC(=O)Nc1c(C#Cc2cc3sc(C4(CO)CC4)cc3s2)cnn1C)c1ccccc1] |
| IT084 | 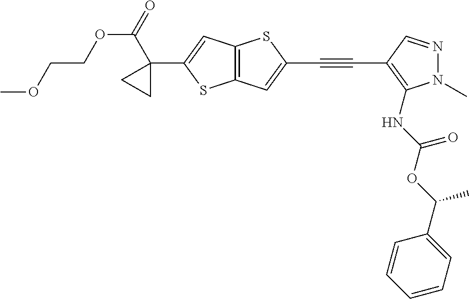 [SMILES: COCCOC(=O)C1(c2cc3sc(C#Cc4cnn(C)c4NC(=O)O[C@H](C)c4ccccc4)cc3s2)CC1] |
| IT085 | 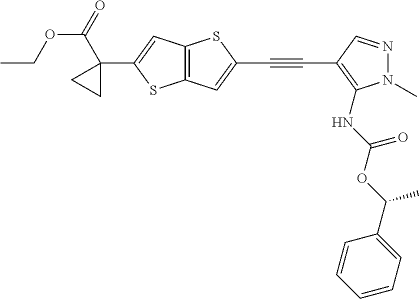 [SMILES: CCOC(=O)C1(c2cc3sc(C#Cc4cnn(C)c4NC(=O)O[C@H](C)c4ccccc4)cc3s2)CC1] |
| IT086 | 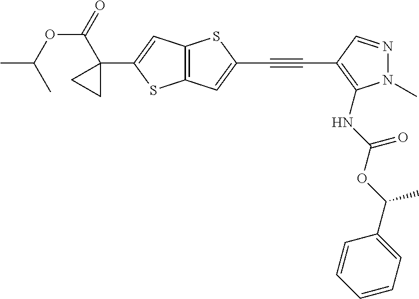 [SMILES: CC(C)OC(=O)C1(c2cc3sc(C#Cc4cnn(C)c4NC(=O)O[C@H](C)c4ccccc4)cc3s2)CC1] |
| IT087 | 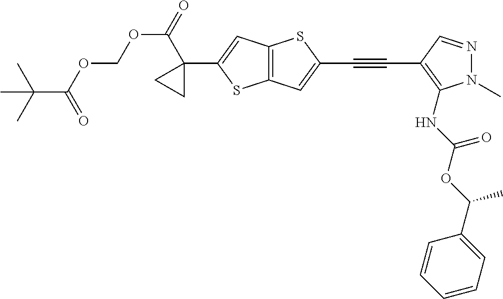 [SMILES: C[C@@H](OC(=O)Nc1c(C#Cc2cc3sc(C4(C(=O)OCOC(=O)C(C)(C)C)CC4)cc3s2)cnn1C)c1ccccc1] |
| IT088 | 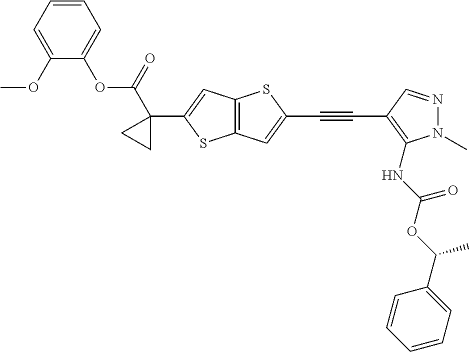 [SMILES: COc1ccccc1OC(=O)C1(c2cc3sc(C#Cc4cnn(C)c4NC(=O)O[C@H](C)c4ccccc4)cc3s2)CC1] |
| IT089 | 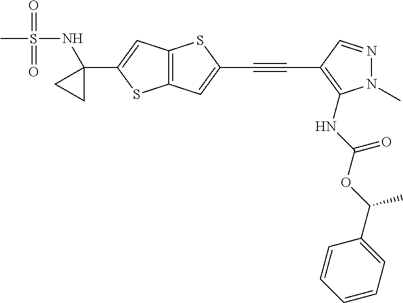 [SMILES: C[C@@H](OC(=O)Nc1c(C#Cc2cc3sc(C4(NS(C)(=O)=O)CC4)cc3s2)cnn1C)c1ccccc1] |
| IT090 | 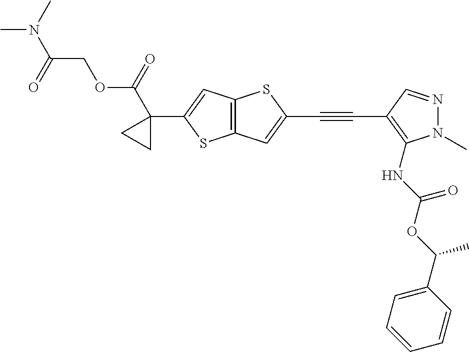 [SMILES: C[C@@H](OC(=O)Nc1c(C#Cc2cc3sc(C4(C(=O)OCC(=O)N(C)C)CC4)cc3s2)cnn1C)c1ccccc1] |
| IT091 | 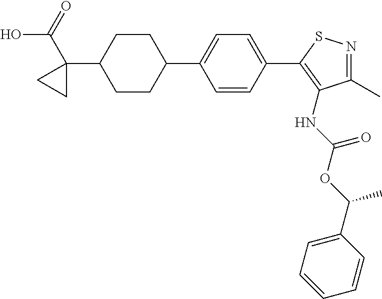 [SMILES: Cc1nsc(-c2ccc(C3CCC(C4(C(=O)O)CC4)CC3)cc2)c1NC(=O)O[C@H](C)c1ccccc1] |
| IT092 | 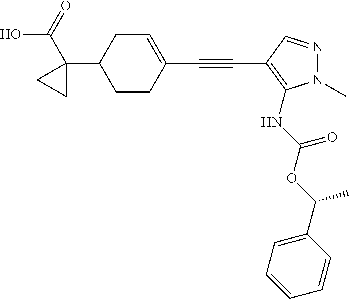 [SMILES: C[C@@H](OC(=O)Nc1c(C#CC2=CCC(C3(C(=O)O)CC3)CC2)cnn1C)c1ccccc1] |
| IT093 | 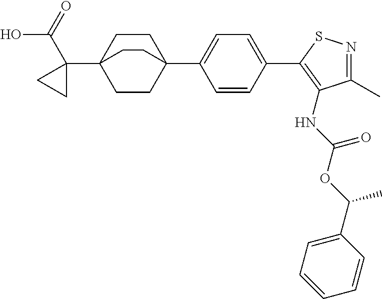 [SMILES: Cc1nsc(-c2ccc(C34CCC(C5(C(=O)O)CC5)(CC3)CC4)cc2)c1NC(=O)O[C@H](C)c1ccccc1] |
| IT094 | 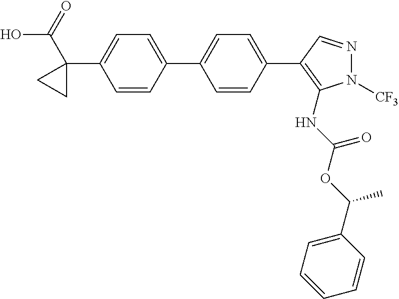 [SMILES: C[C@@H](OC(=O)Nc1c(-c2ccc(-c3ccc(C4(C(=O)O)CC4)cc3)cc2)cnn1C(F)(F)F)c1ccccc1] |
| IT095 | 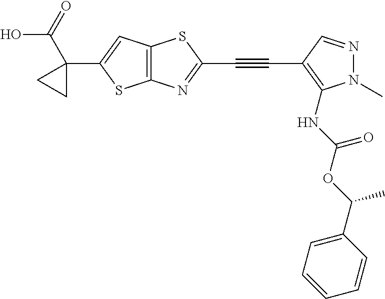 [SMILES: C[C@@H](OC(=O)Nc1c(C#Cc2nc3sc(C4(C(=O)O)CC4)cc3s2)cnn1C)c1ccccc1] |
| IT096 | 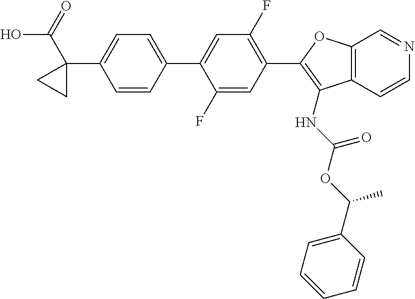 [SMILES: C[C@@H](OC(=O)Nc1c(-c2cc(F)c(-c3ccc(C4(C(=O)O)CC4)cc3)cc2F)oc2cnccc12)c1ccccc1] |
| IT097 | 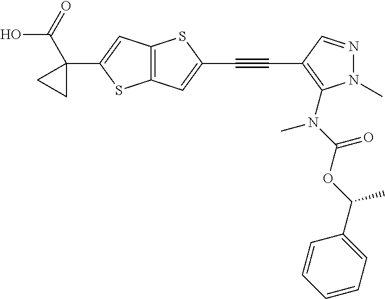 [SMILES: C[C@@H](OC(=O)N(C)c1c(C#Cc2cc3sc(C4(C(=O)O)CC4)cc3s2)cnn1C)c1ccccc1] |
| IT098 | 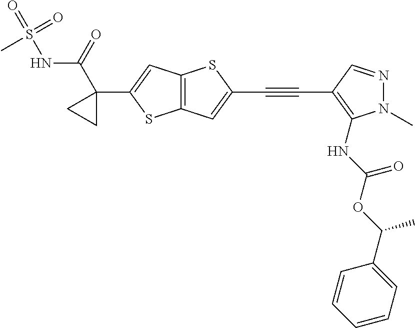 [SMILES: C[C@@H](OC(=O)Nc1c(C#Cc2cc3sc(C4(C(=O)NS(C)(=O)=O)CC4)cc3s2)cnn1C)c1ccccc1] |
| IT099 | 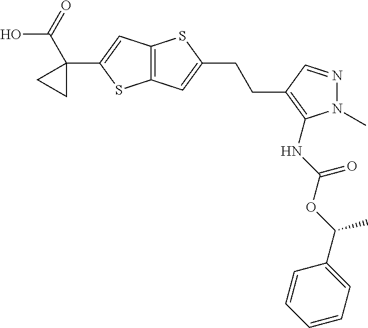 [SMILES: C[C@@H](OC(=O)Nc1c(CCc2cc3sc(C4(C(=O)O)CC4)cc3s2)cnn1C)c1ccccc1] |
| IT100 | 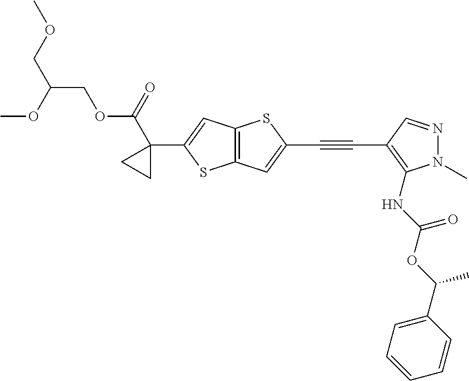 [SMILES: COCC(COC(=O)C1(c2cc3sc(C#Cc4cnn(C)c4NC(=O)O[C@H](C)c4ccccc4)cc3s2)CC1)OC] |
| IT101 | 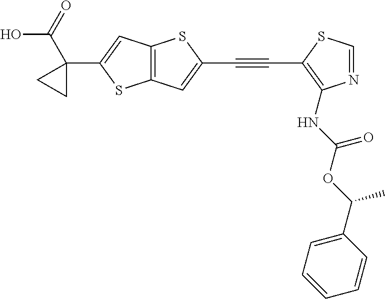 [SMILES: C[C@@H](OC(=O)Nc1ncsc1C#Cc1cc2sc(C3(C(=O)O)CC3)cc2s1)c1ccccc1] |
| IT102 | 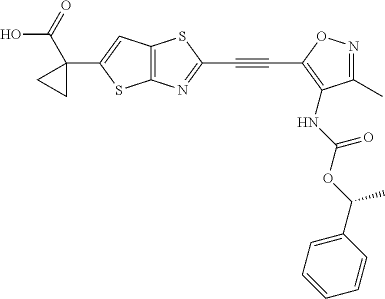 [SMILES: Cc1noc(C#Cc2nc3sc(C4(C(=O)O)CC4)cc3s2)c1NC(=O)O[C@H](C)c1ccccc1] |
| IT103 | 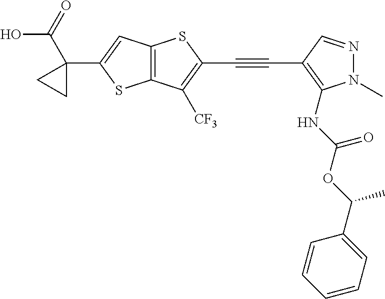 [SMILES: C[C@@H](OC(=O)Nc1c(C#Cc2sc3cc(C4(C(=O)O)CC4)sc3c2C(F)(F)F)cnn1C)c1ccccc1] |

Diseases, Disorders and Conditions Associated with LPA Activity

The compounds of preferred embodiments inhibit the physiological activity of LPA. As such the compounds of preferred embodiments are useful as agents for the treatment or prevention of diseases in which inhibition of the physiological activity of LPA is desirable, such as in the treatment of diseases in which an LPA receptor participates, or is involved in the etiology or pathology of the disease, or is otherwise associated with at least one symptom of the disease. The compounds of preferred embodiments can be employed for the treatment or prevention of side effects, complications, or adverse events associated with the use of a conventional therapeutic agent or therapeutic action (e.g., surgery, etc.) used in treating a disease or condition in which inhibition of LPA physiological activity is desirable. The compounds of preferred embodiments are antagonists of at least one of the LPA receptors, e.g., LPA₁, LPA₂, LPA₃, LPA₄, LPA₅, and/or LPA₆. Certain of the compounds of preferred embodiments are selective antagonists for one or more of the LPA receptors relative to the other LPA receptors.

The compounds of preferred embodiments are used in the treatment of diseases, disorders, or conditions in which activation of at least one LPA receptor by LPA contributes to the symptomology or progression of the disease, disorder, or condition. The compounds of preferred embodiments are antagonists of LPA receptor(s). Diseases, disorders, or conditions that the compounds of preferred embodiments can be used to treat include, but are not limited to, fibrosis, cancer, or respiratory disorders. For examples, the fibrosis can include pulmonary fibrosis, dermal fibrosis, kidney fibrosis, or liver fibrosis. In one embodiment, the fibrosis is idiopathic pulmonary fibrosis.

The terms “fibrosis” or “fibrosing disorder,” as used herein, are broad terms and refer without limitation to conditions that are associated with the abnormal accumulation of cells and/or fibronectin and/or collagen and/or increased fibroblast recruitment and include but are not limited to fibrosis of individual organs or tissues such as the lung. Exemplary diseases, disorders, or conditions that involve fibrosis include, but are not limited to, idiopathic pulmonary fibrosis.

LPA and LPA₁ play key pathogenic roles in pulmonary fibrosis. Fibroblast chemoattractant activity plays a role in the lungs in patients with pulmonary fibrosis. Profibrotic effects of LPA₁-receptor stimulation is explained by LPA₁-receptor-mediated vascular leakage and increased fibroblast recruitment, both profibrotic events. The LPA-LPA₁ pathway has a role in mediating fibroblast migration and vascular leakage in IPF. The end result is the aberrant healing process that characterizes this fibrotic condition. The LPA-LPA₂ pathway contributes to the activation of the TGF-β pathway in pulmonary fibrosis. Compounds that inhibit LPA₂ may show efficacy in the treatment of lung fibrosis. Compounds that inhibit both LPA₁ and LPA₂ may show improved efficacy in the treatment of lung fibrosis compared to compounds which inhibit only LPA₁ or LPA₂.

Some embodiments described herein relate to a method of treating a fibrotic condition, which can include administering a therapeutically effective amount of a compound disclosed herein, or a pharmaceutically acceptable salt thereof, to a subject. The methods include identifying a subject at risk for or having a fibrotic condition and administering a compound to the subject in an effective amount for therapeutic treatment or prophylactic treatment of the fibrotic condition.

A “fibrotic condition,” “fibroproliferative condition,” “fibrotic disease,” “fibroproliferative disease,” “fibrotic disorder,” and “fibroproliferative disorder” are used interchangeably to refer to a condition, disease or disorder that is characterized by dysregulated proliferation or activity of fibroblasts and/or abnormal accumulation of fibronectin and/or pathologic or excessive accumulation of collagenous tissue. Typically, any such disease, disorder or condition is amenable to treatment by administration of a compound having anti-fibrotic activity. Fibrotic disorders include, but are not limited to, pulmonary fibrosis, including idiopathic pulmonary fibrosis (IPF) and pulmonary fibrosis from a known etiology, dermal fibrosis, pancreatic fibrosis, liver fibrosis (e.g., hepatic fibrosis associated with chronic active hepatitis), and renal fibrosis.

In some embodiments, the subject is a human.

The terms “therapeutically effective amount,” as used herein, refer to an amount of a compound sufficient to cure, ameliorate, slow progression of, prevent, or reduce the likelihood of onset of the identified disease or condition, or to exhibit a detectable therapeutic, prophylactic, or inhibitory effect. The effect can be detected by, for example, the assays disclosed in the following examples. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

For any compound, the therapeutically or prophylactically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. However, pharmaceutical compositions that exhibit narrow therapeutic indices are also within the scope of the invention. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In one aspect, treating a condition described herein results in an increase in average survival time of a population of treated subjects in comparison to a population of untreated subjects. Preferably, the average survival time is increased by more than about 30 days; more preferably, by more than about 60 days; more preferably, by more than about 90 days; and even more preferably by more than about 120 days. An increase in survival time of a population may be measured by any reproducible means. In a preferred aspect, an increase in average survival time of a population may be measured, for example, by calculating for a population the average length of survival following initiation of treatment with an active compound. In an another preferred aspect, an increase in average survival time of a population may also be measured, for example, by calculating for a population the average length of survival following completion of a first round of treatment with an active compound.

In another aspect, treating a condition described herein results in a decrease in the mortality rate of a population of treated subjects in comparison to a population of subjects receiving carrier alone. In another aspect, treating a condition described herein results in a decrease in the mortality rate of a population of treated subjects in comparison to an untreated population. In a further aspect, treating a condition described herein results a decrease in the mortality rate of a population of treated subjects in comparison to a population receiving monotherapy with a drug that is not a compound of the embodiments, or a pharmaceutically acceptable salt, metabolite, analog or derivative thereof. Preferably, the mortality rate is decreased by more than about 2%; more preferably, by more than about 5%; more preferably, by more than about 10%; and most preferably, by more than about 25%. In a preferred aspect, a decrease in the mortality rate of a population of treated subjects may be measured by any reproducible means. In another preferred aspect, a decrease in the mortality rate of a population may be measured, for example, by calculating for a population the average number of disease-related deaths per unit time following initiation of treatment with an active compound. In another preferred aspect, a decrease in the mortality rate of a population may also be measured, for example, by calculating for a population the average number of disease related deaths per unit time following completion of a first round of treatment with an active compound.

In another aspect, treating a condition described herein results in a reduction in the rate of cellular proliferation. Preferably, after treatment, the rate of cellular proliferation is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The rate of cellular proliferation may be measured by any reproducible means of measurement. In a preferred aspect, the rate of cellular proliferation is measured, for example, by measuring the number of dividing cells in a tissue sample per unit time.

In another aspect, treating a condition described herein results in a reduction in the proportion of proliferating cells. Preferably, after treatment, the proportion of proliferating cells is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The proportion of proliferating cells may be measured by any reproducible means of measurement. In a preferred aspect, the proportion of proliferating cells is measured, for example, by quantifying the number of dividing cells relative to the number of nondividing cells in a tissue sample. In another preferred aspect, the proportion of proliferating cells is equivalent to the mitotic index.

In another aspect, treating a condition described herein results in a decrease in size of an area or zone of cellular proliferation. Preferably, after treatment, size of an area or zone of cellular proliferation is reduced by at least 5% relative to its size prior to treatment; more preferably, reduced by at least about 10%; more preferably, reduced by at least about 20%; more preferably, reduced by at least about 30%; more preferably, reduced by at least about 40%; more preferably, reduced by at least about 50%; even more preferably, reduced by at least about 60%; and most preferably, reduced by at least about 75%. Size of an area or zone of cellular proliferation may be measured by any reproducible means of measurement. In a preferred aspect, size of an area or zone of cellular proliferation may be measured as a diameter or width of an area or zone of cellular proliferation.

The methods described herein may include identifying a subject in need of treatment. In a preferred embodiment, the methods include identifying a mammal in need of treatment. In a highly preferred embodiment, the methods include identifying a human in need of treatment. Identifying a subject in need of treatment may be accomplished by any means that indicates a subject who may benefit from treatment. For example, identifying a subject in need of treatment may occur by clinical diagnosis, laboratory testing, or any other means known to one of skill in the art, including any combination of means for identification.

As described elsewhere herein, the compounds described herein may be formulated in pharmaceutical compositions, if desired, and can be administered by any route that permits treatment of the disease or condition. A preferred route of administration is oral administration. Administration may take the form of single dose administration, or the compound of the embodiments can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

### Pharmaceutical Compositions/Formulations, Routes of Administration, and Methods of Treatment

In some embodiments, the compounds described herein are prepared into pharmaceutical compositions. Pharmaceutical compositions suitable for administration to a patient in need thereof can be prepared using techniques known in the art. Pharmaceutically acceptable inactive ingredients that facilitate processing of the active compounds into pharmaceutical compositions can also be employed. Once a route of administration chosen, a pharmaceutical composition can be developed. Suitable pharmaceutical compositions include those described, e.g., in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), the contents of which are hereby expressly incorporated by reference herein.

Pharmaceutical compositions suitable for use in the methods of preferred embodiments include a mixture of one or more compounds of a preferred embodiment with other chemical components (e.g., pharmaceutically acceptable inactive or active ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to a patient in need thereof.

The pharmaceutical compositions of preferred embodiments can be systemically and/or locally administrable to a patent in need thereof in a variety of ways and by multiple administration routes, including, but not limited to, oral, parenteral (e.g., intravenous, subcutaneous, intramuscular, intramedullary injections, intrathecal, direct intraventricular, intraperitoneal, intralymphatic, intranasal injections), inhalation, injection (e.g., intramuscular, subcutaneous, or intravenous), rectal (e.g., enemas, rectal gels, rectal foams, rectal aerosols, suppositories, jelly suppositories, or retention enemas), intranasal, buccal, topical or transdermal administration routes. Such pharmaceutical compositions can be in a form of aqueous liquid dispersions, aqueous oral dispersions, emulsions, solutions, elixirs, gels, syrups, self-emulsifying dispersions, solid solutions, liposomal dispersions, aerosols, mists, solid dosage forms, powders, nasal sprays, nasal mists, eye drops immediate release formulations, controlled release formulations, fast melt formulations, tablets, lozenge, capsules, pills, delayed release formulations, extended release formulations, pulsatile release formulations, multiparticulate formulations, and mixed immediate and controlled release formulations. Topically administrable compositions include solutions, suspensions, lotions, gels, pastes, shampoos, scrubs, rubs, smears, medicated sticks, medicated bandages, balms, creams, or ointments.

The compounds of preferred embodiments and pharmaceutical compositions comprising the same can be used for treating, preventing, reversing, halting or slowing the progression of LPA-dependent or LPA-mediated diseases or conditions once it becomes clinically evident, or treating the symptoms associated with or related to LPA-dependent or LPA-mediated diseases or conditions, by administering the compound to a subject in need thereof, e.g., a subject that has a LPA-dependent or LPA-mediated disease or condition at the time of administration, or is at risk of developing a LPA-dependent or LPA-mediated disease or condition.

Also provided are methods that include the diagnosis or determination of whether or not a patient is suffering from a LPA-dependent or LPA-mediated disease or condition by administering to the subject a therapeutically effective amount of a compound of a preferred embodiment and determining whether or not the patient responds to the treatment.

The pharmaceutical compositions can be administered continuously or intermittently, e.g., in single administrations of an effective amount of the compound, or administrations twice, three times, or four times or more over the span of one day. The pharmaceutical compositions can be administered over a single day or multiple days, with a time between administrations of, e.g., 4, 5, 6, 7, 8, 9, 10, 11, 12 or 24 hours. For example, the compound of preferred embodiments can be administered continuously or intermittently as in a single dose; or in multiple doses with a dose administered every 6 hours, or 8 hours, or 12 hours, or 24 hours. Also contemplated are administration methods including a drug holiday, wherein the administration of the compound is temporarily suspended or the dose of the compound being administered is temporarily reduced; at the end of the drug holiday, dosing of the compound is resumed. The length of the drug holiday varies from 2 days, or 1 month, or two months, or 3 months, or 6 months, or 9 months, to 1 year or more. The pharmaceutical composition can be administered therapeutically or prophylactically for a fixed period of time indefinitely.

The compounds of preferred embodiments can be used in the preparation of medicaments for the treatment of LPA-dependent or LPA-mediated diseases or conditions. Treatment involves administration of pharmaceutical compositions that include at least one compound of preferred embodiments or a pharmaceutically acceptable salt, active metabolite, prodrug, or solvate thereof, in a therapeutically effective amount, to said patient. The compounds of preferred embodiments can be administered for prophylactic and/or therapeutic treatment. In certain therapeutic applications, the compositions are administered to a patient already suffering from a disease or condition, in an amount sufficient to cure or at least partially mitigate at least one of the symptoms of the disease or condition. Amounts effective for this use depend on the severity and course of the disease or condition, previous therapy, the patient's health status, weight, and response to the drugs, and the judgment of the treating physician. Therapeutically effective amounts can be determined by methods including, but not limited to, a dose escalation clinical trial. In prophylactic applications, the compounds of preferred embodiments are administered to a patient susceptible to or otherwise at risk of a particular disease, disorder, or condition. The dose of drug being administered may be temporarily reduced or temporarily suspended for a certain length of time (i.e., a “drug holiday”).

Doses employed for adult human treatment are typically in the range of 0.01 mg to 5000 mg per day, or from about 1 mg to about 1000 mg per day. The desired dose can be provided in a single dose or in divided doses.

In certain embodiments, patients in need of treatment can be identified by screening for LPA receptor gene SNPs. Patients can be further selected based on increased LPA receptor expression in the tissue of interest. LPA receptor expression are determined by methods including, but not limited to, northern blotting, western blotting, quantitative PCR (qPCR), flow cytometry, autoradiography (using a small molecule radioligand or PET ligand). In some embodiments, patients are selected based on the concentration of serum or tissue LPA measured by mass spectrometry. In some embodiments, patients are selected based on a combination of the above markers (increased LPA concentrations and increased LPA receptor expression).

In certain embodiments, the compounds of preferred embodiments are administered with another therapeutic treatment or another therapeutic agent, e.g., a second therapeutic agent that modulates different aspects of the disease, disorder or condition being treated, thereby providing a greater overall benefit than administration of either therapeutic agent alone. For combination therapies, the dosages of the co-administered compounds vary depending on the type or specific drug employed, on the disease or condition being treated, and other factors. When co-administered with one or more other therapeutic agents, the compounds of preferred embodiments can be administered either simultaneously with the one or more other therapeutic agents, or sequentially, and can be present in the same unit dosage form or in different unit dosage forms. If administration is simultaneous, the multiple therapeutic agents are, by way of example only, provided in a single, unified form, or in multiple forms. In the treatment of cancer, it is advantageous to administer a compound of a preferred embodiment in combination with one or more anti-cancer agents and/or radiation therapy. In the treatment of fibrosis, it is advantageous to administer a compound of a preferred embodiment in combination with one or more immunosuppressant and/or with corticosteroids. In treating LPA-dependent or LPA-mediated conditions or diseases, such as the therapy of respiratory disorders (e.g., pulmonary fibrosis, asthma, COPD, rhinitis), it is advantageous to administer a compound of a preferred embodiment in combination with one or more agents used in the treatment of respiratory conditions, e.g., anti-inflammatory agents or inhaled corticosteroids.

### Synthesis

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Some embodiments described herein relate to method of preparing compounds of formula (VIIb), comprising conducting a palladium catalyzed cross-coupling reaction between a compound of formula (VII-1) and a compound of formula (VII-3) as shown in Scheme 1 below. Alternatively, compounds of formula (VIIb) can be prepared by conducting a palladium catalyzed cross-coupling reaction between a compound of formula (VII-2) and a compound of formula (VII-4) as shown in Scheme 2 below:

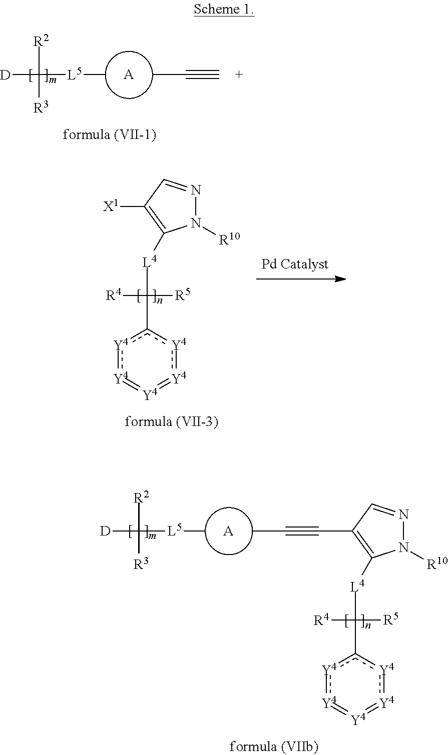

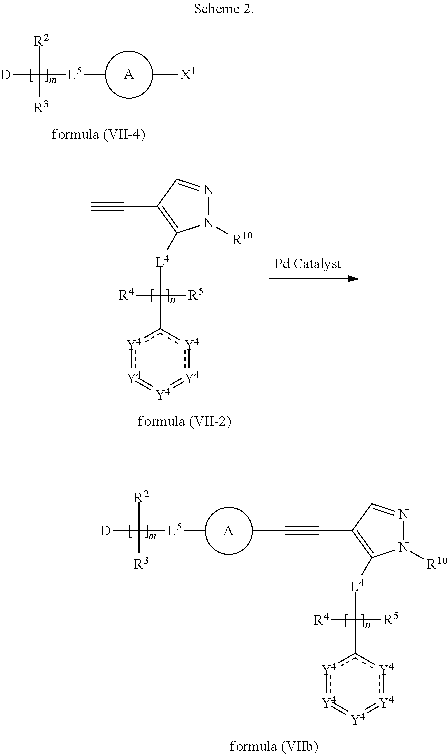

wherein X¹ is a halogen selected from Br or I;

A is a ring system selected from the group consisting of

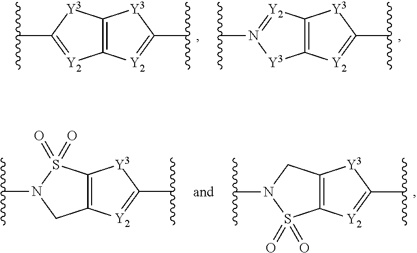

wherein A is unsubstituted or substituted with one or more substituents selected from alkyl, halogen, haloalkyl, hydroxy, alkoxy, haloalkoxy, cyano, or oxo;

D is selected from —OH,

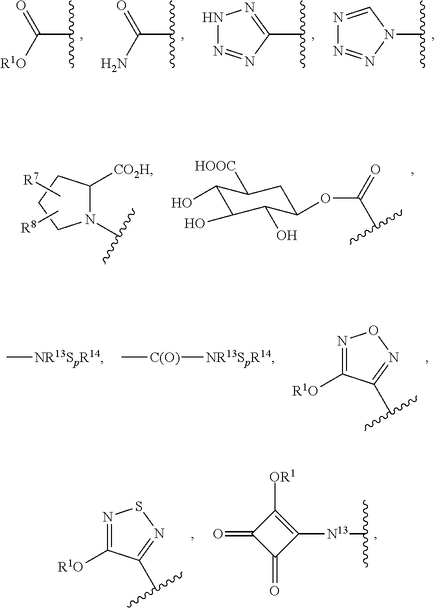

or carboxylic acid isosteres;

L⁵ is selected from a single bond, a —CH₂O— linker, a —CH═CH— linker, a —C≡C— linker, a

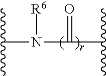

linker, or a 4-7 membered heterocyclyl;

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶ is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ is independently selected from hydrogen, alkyl, haloalkyl, aryl, or C₃₋₆ cycloalkyl;

each R¹⁴ is independently selected from hydrogen, alkyl, haloalkyl, aryl, or C₃₋₆ cycloalkyl;

L⁴ is

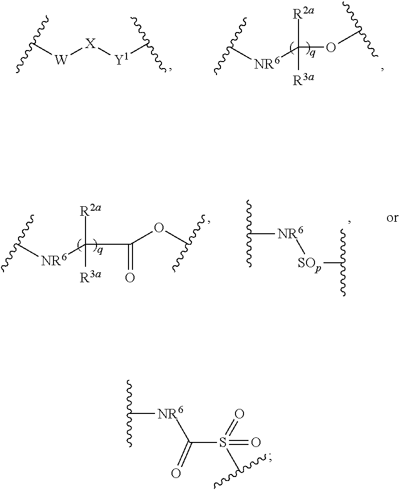

R^{2a} and R^{3a} are each independently selected from hydrogen, alkyl, aryl, or heteroaryl; or R^{2a} and R^{3a} are joined together with the atom to which they are attached to form an optionally substituted cycloalkyl or an optionally substituted heterocyclyl;

W is selected from C(R⁶)₂, NR⁶, or O;

X is selected from —C(O) or S(O)ₚ;

Y¹ is selected from C(R⁶)₂, NR⁶, or O;

Y² is selected from —CH═ or N;

Y³ is selected from C(R⁶)₂, NR⁶, O or S;

each Y⁴ is independently absent, CR⁹, C(R⁹)₂, N, or NH, provided that only one Y⁴ can be absent;

m is an integer from 0-3;

n is an integer from 0-3;

p is an integer from 1-2;

q is an integer from 1-6;

r is an integer of 0 or 1; and

[symbol] represents a single or double bond.

In some embodiments, the compound of formula (VII-3) is also represented by formula (VII-3A):

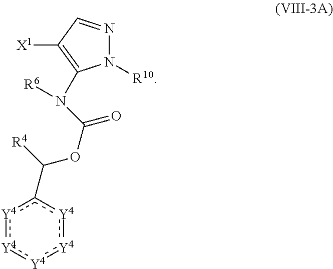

In some embodiments, the compound of formula (VII-4) is also represented by formula (VII-4A):

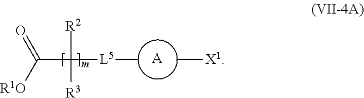

In some embodiments, A is selected from

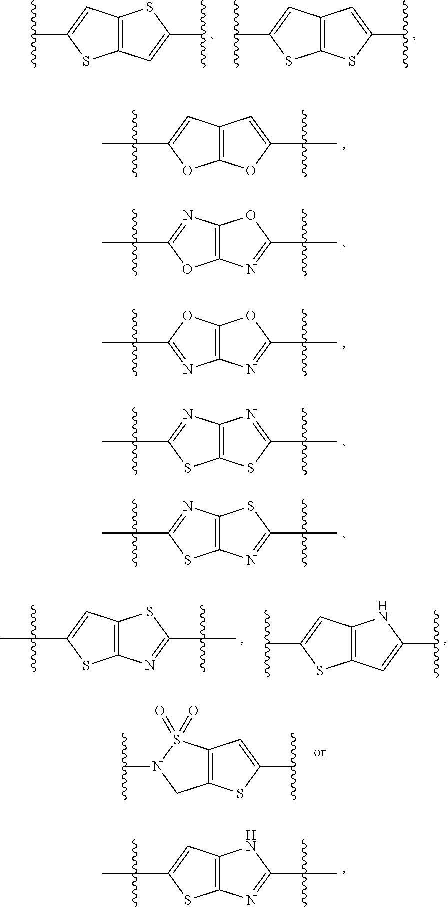

, and wherein the rings in A are unsubstituted or substituted with one or more substituents selected from alkyl, haloalkyl, halogen, hydroxy, alkoxy, haloalkoxy, cyano, or oxo. In some such embodiments, A is selected from

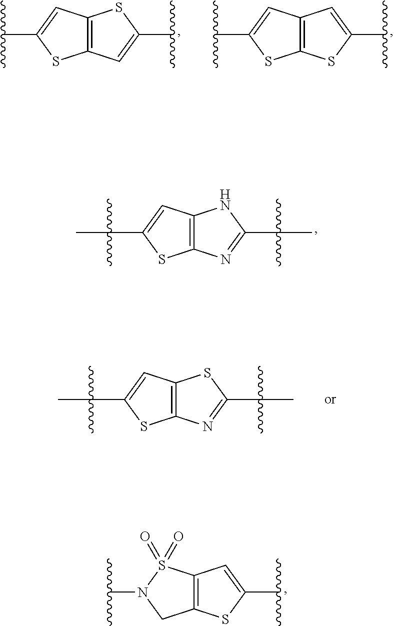

each

optionally substituted with one or more substituents selected from the group consisting of alkyl, alkoxy, halogen, haloalkyl and cyano.

In some embodiments, R¹ is hydrogen or unsubstituted alkyl. In some other embodiments, R¹ is alkyl substituted with one or more substituents selected from the group consisting of alkoxy, C-amido, O-carboxy, and 6 membered heterocyclyl. In still some other embodiments, R¹ is optionally substituted aryl.

In some embodiments, m is 0. In some other embodiments, m is 1. In still some other embodiments, m is 2.

In some embodiments, R² and R³ is hydrogen. In some other embodiments, one of R² and R³ is hydrogen and the other R² and R³ is aryl. In still some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond.

In some embodiments, R⁶ is hydrogen. In some other embodiments, R⁶ is C₁₋₃ alkyl.

In some embodiments, R¹⁰ is selected from C₁₋₃ alkyl or C₃₋₆ cycloalkyl.

In some embodiments, R⁴ is hydrogen. In some other embodiments, R⁴ is alkyl. In some further such embodiments, R⁴ is alkyl substituted with halogen.

In some embodiments,

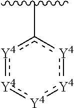

is also represented by

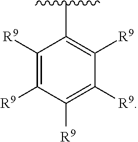

In some further embodiments, each of R⁹ is hydrogen. In some other embodiments, at least one R⁹ is selected from C₁₋₃ alkyl or halogen.

In some embodiments,

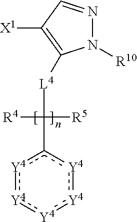

is selected from

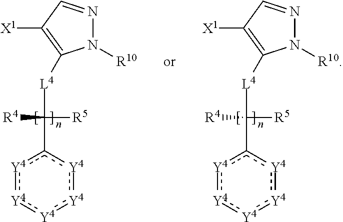

In some embodiments,

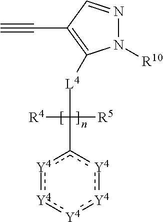

is selected from

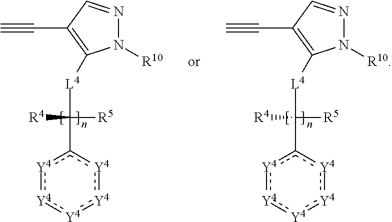

Some embodiments disclosed herein relate to compounds of formula (VII-1), wherein the structure of formula (VII-1) and the variables thereof including ring A, D, R¹, R², R³, R⁶, R⁷, R⁸, R¹³, R¹⁴, L⁵, Y², Y³, m, p and r are defined above in formula (VIIb).

In some embodiments, the compound of formula (VII-1) is also represented by formula (VII-1A):

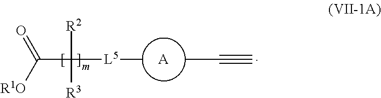

In some embodiments, ring A is selected from

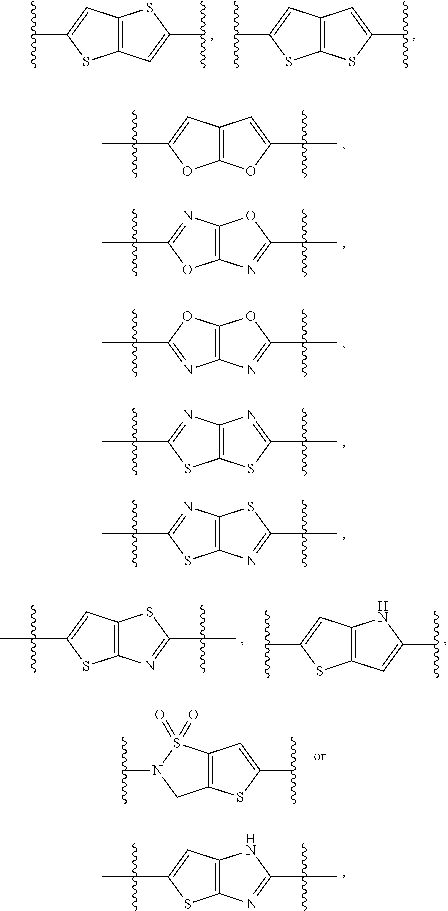

and wherein the rings in A are unsubstituted or substituted with one or more substituents selected from alkyl, haloalkyl, halogen, hydroxy, alkoxy, haloalkoxy, cyano, or oxo. In some further embodiments, ring A is selected from the group consisting of

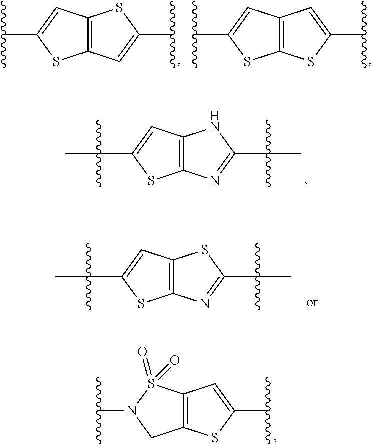

each optionally substituted with one or more substituents selected from the group consisting of alkyl, alkoxy, halogen, haloalkyl and cyano. In some embodiments, R¹ is hydrogen or unsubstituted alkyl. In some other embodiments, R¹ is alkyl substituted with one or more substituents selected from the group consisting of alkoxy, C-amido, O-carboxy, and 6 membered heterocyclyl. In still some other embodiments, R¹ is optionally substituted aryl.

In some embodiments, m is 0. In some other embodiments, m is 1. In still some other embodiments, m is 2.

In some embodiments, R² and R³ is hydrogen. In some other embodiments, one of R² and R³ is hydrogen and the other R² and R³ is aryl. In still some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond.

In some embodiments, the acetylene group of the compound of formula (VII-1) or (VII-1A) is first activated by reacting with a tin reagent. In one embodiment, the tin reagent is n-Bu₃SnCl.

Some embodiments disclosed herein relate to compounds of formula (VII-2), wherein the structure of formula (VII-2) and the variables thereof including R⁴, R⁵, R⁶, R⁹, R¹⁰, L⁴, Y¹, Y⁴, W, X, n, p and q are defined above in formula (VIIb); and wherein R² and R³ are each independently selected from hydrogen, alkyl, aryl, or heteroaryl; or R² and R³ are joined together with the atom to which they are attached to form an optionally substituted cycloalkyl or an optionally substituted heterocyclyl.

In some embodiments, the compound of formula (VII-2) is also represented by formula (VII-2A):

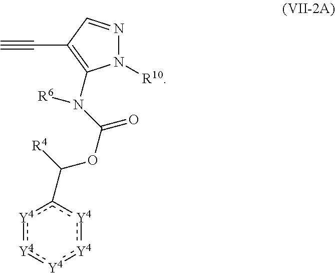

In some embodiments, R⁶ is R⁶ⁱ s hydrogen. In some other embodiments, R is C₁₋₃ alkyl.

In some embodiments, R¹⁰ is selected from C₁₋₃ alkyl or C₃₋₆ cycloalkyl.

In some embodiments, R⁴ is hydrogen. In some other embodiments, R⁴ is alkyl. In some further such embodiments, R⁴ is alkyl substituted with halogen.

In some embodiments,

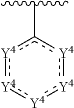

is also represented by

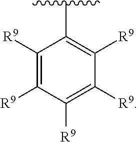

In some further embodiments, each of R⁹ is hydrogen. In some other embodiments, at least one R⁹ is selected from C₁₋₃ alkyl or halogen.

In some embodiments

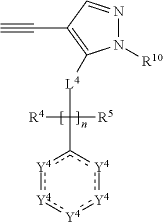

is selected from

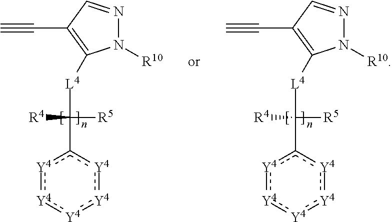

### EXAMPLES

Additional embodiments are disclosed in further detail in the following examples, which are not in any way intended to limit the scope of the claims.

### Example 1-A

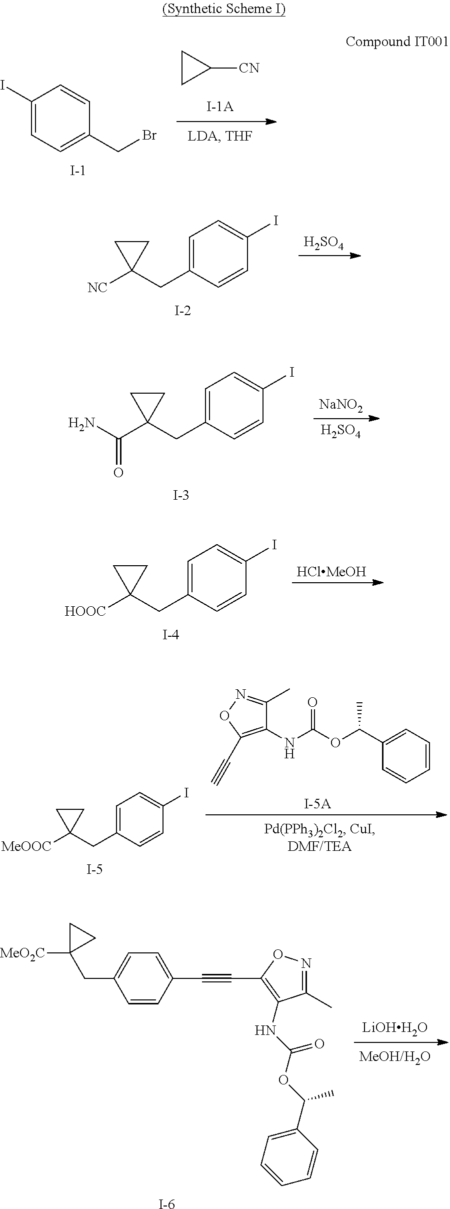

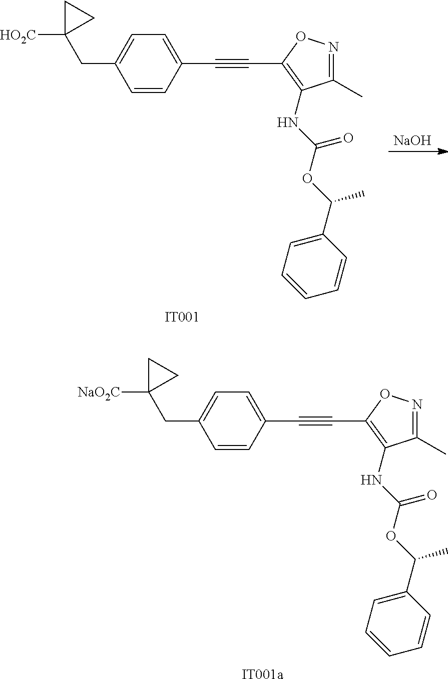

n-Butyl lithium (1.35 mL, 2.5M, 3.38 mmol) was added to a solution of diisopropylamine (0.45 mL, 3.24 mmol) in THF at −78° C. After 35 min, a solution of I-1A (189 mg, 2.82 mmol) in THF was added, and the bright yellow solution was stirred for 20 min. Then a solution of I-1 (1.0 g, 3.38 mmol) in THF (5 mL) was then added dropwise, and the reaction mixture was stirred with warming to rt over 1 h. The mixture was partitioned between water and EtOAc, dried over Na₂SO₄, and purified by column on chromatograph (PE:EA=100:3) to afford I-2 (250 mg, yield 31.3%).

The solution of I-2 (250 mg, 0.88 mmol) in con.H₂SO₄ (12 mL) was heated to 100° C. for 2 hrs. The mixture was poured into ice-water and extracted with EtOAc. The organic layer was washed with H₂O, dried and concentrated to give I-3 (250 mg, crude yield 100%), which was used to next step directly.

To a stirred solution of I-3 (250 mg, 0.83 mmol) in con.H₂SO₄ (12 mL) was added in portions NaNO₂ (573 mg, 8.3 mmol) at 0° C. After addition, the mixture was heated to 100° C. for 2 hrs. The mixture was poured into ice-water and extracted with EtOAc. The organic layer was washed with H₂O, dried and concentrated to give I-4 (250 mg, crude yield 100%), which was used to next step directly.

The solution of I-4 (250 mg, 0.83 mmol) in MeOH/HCl (10 mL) was heated to 60° C. overnight. After concentrated, the residue was extracted with EtOAc, washed with aq. NaHCO₃ and brine. The mixture was poured into ice-water to afford a white precipitate. The organic layer was dried and concentrated to give I-5 (120 mg, yield 45.8%). MS (ESI) m/z (M+H)⁺316.9.

To a stirred mixture of I-5 (50 mg, 0.165 mmol), I-5A (44.4 mg, 0.165 mmol) and CuI (1.6 mg, 0.008 mmol) in DMF (3 mL) and TEA (1 mL) was added Pd(PPh₃)₂Cl₂ (12 mg, 0.02 mmol). The reaction mixture was flushed with Ar and stirred at rt for overnight. The mixture was diluted with EtOAc (20 mL), washed with water and brine. The organic layer was dried over Na₂SO₄, filtered and concentrated. The residue was purified by prep-TLC (PE:EA=5:1) to give I-6 (20 mg, yield 28.7%). MS (ESI) m/z (M+Na)⁺ 481.1.

To a solution of I-6 (46 mg, 0.10 mmol) in MeOH (6.0 mL) was added water (2.0 mL) and lithium monohydrate (21.2 mg, 0.50 mmol). The reaction mixture was stirred at rt overnight. The mixture was adjust to pH=4.0 with 1N hydrochloride solution, and extracted with EtOAc. The combined organic phase was dried over MgSO₄ and concentrated. The residue was purified by prep-HPLC to give IT001 (45 mg, yield 100%). MS (ESI) m/z (M+H)⁺ 445.1.

To a solution of I-7 (46 mg, 0.104 mmol) in MeOH (2 mL) was added 0.05N NaOH solution (2.08 mL). The reaction mixture was stirred for 30 minutes. The mixture was lyophilized to give IT001a. ¹H NMR (400 MHz, Methanol-d₄): δ 7.31-7.41 (m, 9H), 5.84 (q, 1H), 3.02 (s, 2H), 2.20 (s, 3H), 1.60 (d, J=6.4 Hz, 3H), 1.15-1.17 (m, 2H), 0.61-0.63 (m, 2H). MS (ESI) m/z (M+H)⁺ 445.1.

### Example 1-B

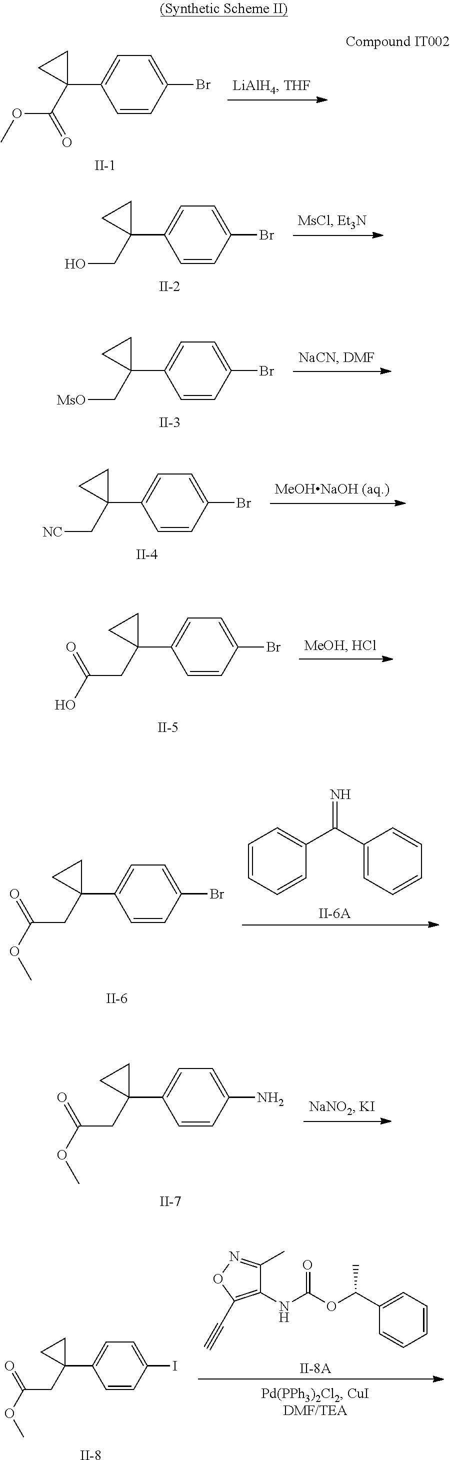

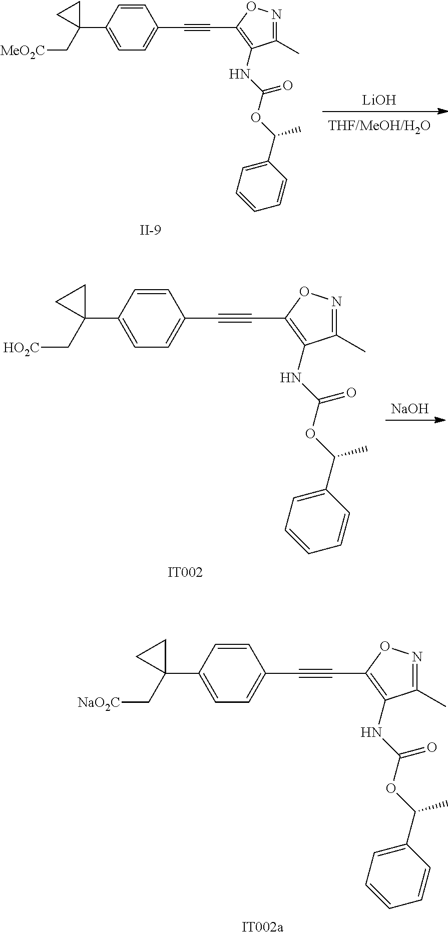

To a solution of II-1 (5 g, 19.7 mmol) in THF (150 mL) at −4° C. was LiAlH₄ (1.54 g, 39.4 mmol) portionwise over 30 min. The reaction was stirred for 30 min, and then water (20 mL) was added, followed by 4N NaOH (15 mL) and additional water (50 mL). The mixture was stirred for 15 minutes and filtered. The filtrate was extract with EtOAc, the combined organic layers were dried over Na₂SO₄, concentrated in vacuo. The residue was purified by column chromatography (PE:EA=5:1) to afford II-2 (3.1 g, yield 70%).

II-2 (5 g 22 mmol) in DCM (80 mL) at −78° C. was treated with Et₃N (4.45 g, 44 mmol), followed by MsCl (2.5 g, 22 mmol). The reaction was stirred for 1 hour at −78° C., and then warmed to 0° C. and stirred for 2 h. The mixture was diluted with 1 N aqueous HCl and extracted with DCM. The combined organic extracts were dried over anhydrous MgSO₄, filtered and concentrated in vacuo. II-3 was used directly without further purification.

The mixture of II-3 (9 g, 29.6 mmol) in DMF (80 mL) was added NaCN (2.78 g, 59.2 mmol), and the reaction mixture was stirred at 70° C. for 3 h. The mixture was diluted with EtOAc and water, and the organic layer was separated, dried and concentrated. The residue was purified by chromatography on silica gel (PE:EA=5:1) to afford II-4 (5.35 g, yield 77%).

The mixture of II-4 (6 g, 22.3 mmol) and NaOH (10 g, 0.25 mol) was dissolved in MeOH (50 mL) and H₂O (50 mL) then the reaction was heated to 60° C. for 16 h. After concentrated, the aqueous layer was adjust to pH=3 with 1N HCl, and extracted with EtOAc, the organic layer was separated, dried and concentrated to afford II-5, which was used in the next step without further purification.

The mixture of II-5 (4 g, 15.7 mmol) in HCl/MeOH (4N, 30 mL) was stirred at reflux for 18 hours. After evaporated of the solvent, the residue was diluted with water and extracted with DCM. The combined organic extracts were dried over anhydrous MgSO₄, filtered and concentrated in vacuo. The residue was purified by column chromatography (PE:EA=10:1) to afford II-6 (2.4 g, yield: 56.8%).

A mixture of II-6 (1 g, 3.72 mmol), II-6A (811 mg, 4.46 mmol), Pd(OAc)₂ (83 mg, 0.37 mmol), BINAP (18 mg, 0.03 mmol) and Cs₂CO₃ (2.4 g, 7.44 mmol) in toluene (120 mL) was vigorously stirred under nitrogen atmosphere at 110° C. for 18 h. After removal of the solvent, the residue was diluted with water and extracted with EtOAc. The combined organic layers were dried over MgSO₄ and evaporated. The residue was purified by column chromatography (PE:EA=10:1) to afford II-7 (0.75 g, yield 55%).

To a solution of p-TsOH (753 mg, 4.38 mmol) in CH₃CN (80 mL) was added II-7 (300 mg, 1.46 mmol). The reaction mixture was cooled to 5° C. and a solution of NaNO₂ (202 mg, 2.93 mmol) and KI (606 mg, 3.65 mmol) in H₂O (9 mL) was added dropwise. The mixture was stirred for 2 h at rt. After removal of the solvent, the residue was diluted with water and extracted with EtOAc. The combined organic layers were dried over MgSO₄ and evaporated. The residue was purified by column chromatography (PE:EA=10:1) to afford II-8 (0.116 g, yield: 25%). MS (ESI) m/z (M+H)⁺317.0.

II-9, IT002, and IT002a were prepared following the similar procedure described in the preparation of I-6, IT001 and IT001a. IT002: MS (ESI) m/z (M+H)⁺445.2. IT002a: ¹H NMR (DMSO-d₆ 300 MHz) δ 7.32-7.38 (m, 9H), 5.76-5.80 (m, 1H), 2.28 (s, 2H), 2.15 (s, 3H), 1.52 (d, J=6.0 Hz, 3H), 0.98 (br, 2H), 0.81 (br, 2H). MS (ESI) m/z (M+H)⁺445.2.

IT003 and IT003a were prepared following the similar synthetic scheme of IT002, using methyl 1-(6-bromonaphthalen-2-yl)cyclopropanecarboxylate in place of II-1. IT003: MS (ESI) m/z (M+H)⁺495.2. IT003a: ¹H NMR (DMSO-d₆, 400 MHz) δ 8.09 (s, 1H), 7.79-7.86 (m, 3H), 7.35-7.53 (m, 7H), 5.80-5.82 (q, 1H), 2.44 (s, 2H), 1.54 (d, J=6.4 Hz, 3H), 1.01 (br, 2H), 0.90 (br, 2H). MS (ESI) m/z (M+H)⁺495.2.

IT065 was prepared following the similar synthetic scheme of IT002, using 1-((6-bromonaphthalen-2-yl)methyl)cyclopropanecarbonitrile in place of II-4, which was obtained in two steps from bromination of (6-bromonaphthalen-2-yl)methanol to form 2-bromo-6-(bromomethyl)naphthalene, followed by reacting with cyclopropanecarbonitrile. IT065: MS (ESI) m/z (M+H)⁺495.1. Sodium salt IT065a: ¹H NMR (400 MHz, Methanol-d₄) δ8.03 (s, 1H), 7.76-7.83 (m, 3H), 7.57 (d, J=8.0 Hz, 1H), 7.27-7.45 (m, 6H), 5.84-5.87 (q, 1H), 3.18 (s, 2H), 2.22 (s, 3H), 1.61 (br, 3H), 1.21 (br, 2H), 0.70 (br, 2H). MS (ESI) m/z (M+H)⁺495.1.

### Example 1-C

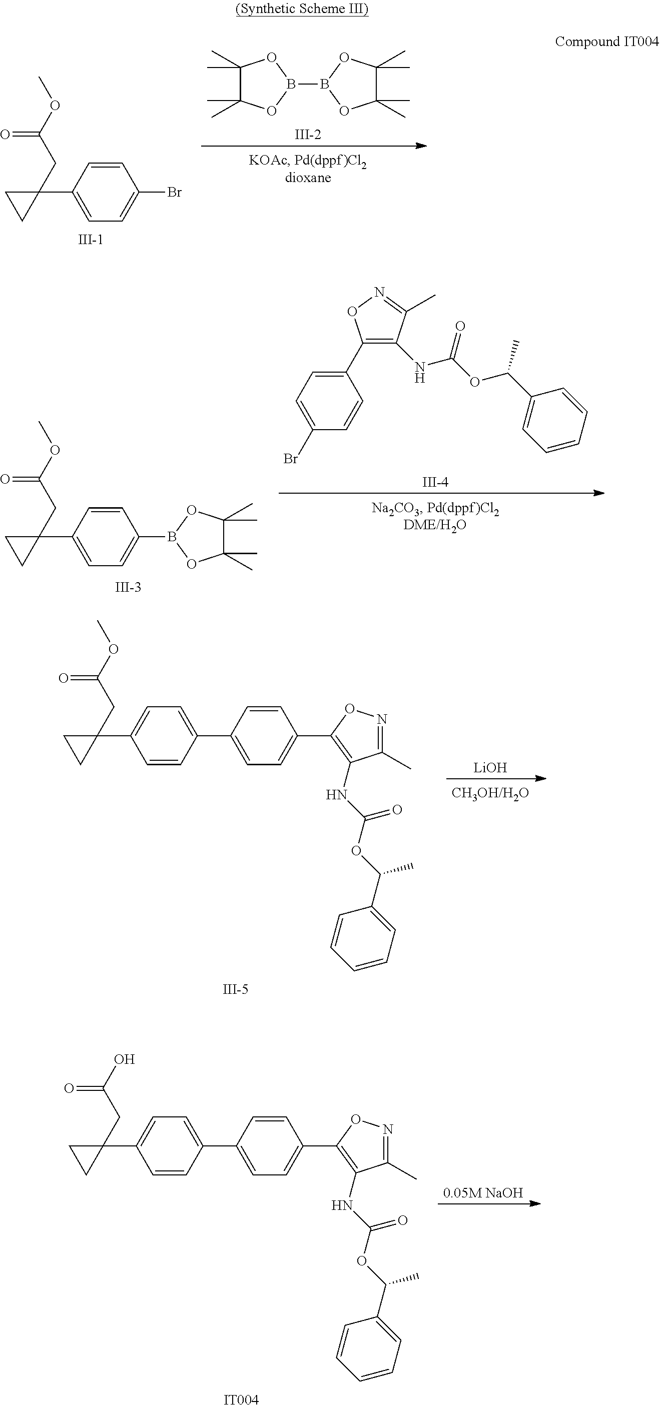

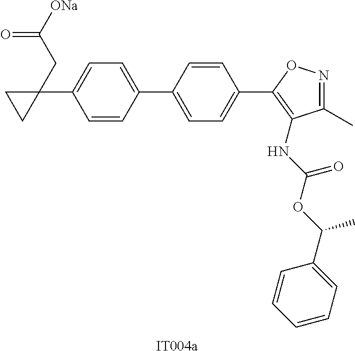

To a stirred solution of III-1 (240 mg, 0.94 mmol), III-2 (286.9 mg, 1.13 mmol), KOAc (184.5 mg, 1.88 mmol) in dioxane (15 mL) was added Pd(dppf)Cl₂ (103.3 mg, 0.14 mmol) The mixture was purged with nitrogen for 5 min and heated to reflux for overnight. After being cooled to rt, the mixture was diluted with water (8 mL) and extracted with EtOAc. The combined organic layers were washed with brine, and concentrated under vacuo. The residue was purified by column chromatography on silica gel (PE:EA=10:1) to give III-3 (190.9 mg, yield 64.2%).

To a stirred solution of III-3 (190.9 mg, 0.64 mmol), III-4 (290.7 mg, 0.72 mmol), Na₂CO₃ (128.1 mg, 1.21 mmol) in DME/H₂O (20 mL, v/v=3:1) was added Pd(dppf)Cl₂ (66.4 mg, 0.09 mmol) under nitrogen. Then the solution was heated to reflux for 4 hours. After concentrated, H₂O (5 mL) was added, and the mixture was extracted with EtOAc. The organic layer was combined and washed with brine, dried over Na₂SO₄, concentrated in vacuo. The residue was purified by column chromatography on silica gel (PE:EA=1:1) to afford III-5 (256 mg, yield: 26.9%).

IT004 and IT004a were prepared following the similar procedure described in the synthesis of IT001 and IT001a. IT004a: ¹H NMR (DMSO-d₆, 400 MHz): δ 7.74-7.77 (m, 4H), 7.54-7.56 (m, 2H), 7.36-7.42 (m, 7H), 5.74-5.75 (q, 1H), 2.32 (br, 2H), 2.12 (s, 3H), 1.54 (br, 2H), 0.96 (br, 2H), 0.79 (br, 2H). MS (ESI) m/z (M+H)⁺497.2.

### Example 2-A

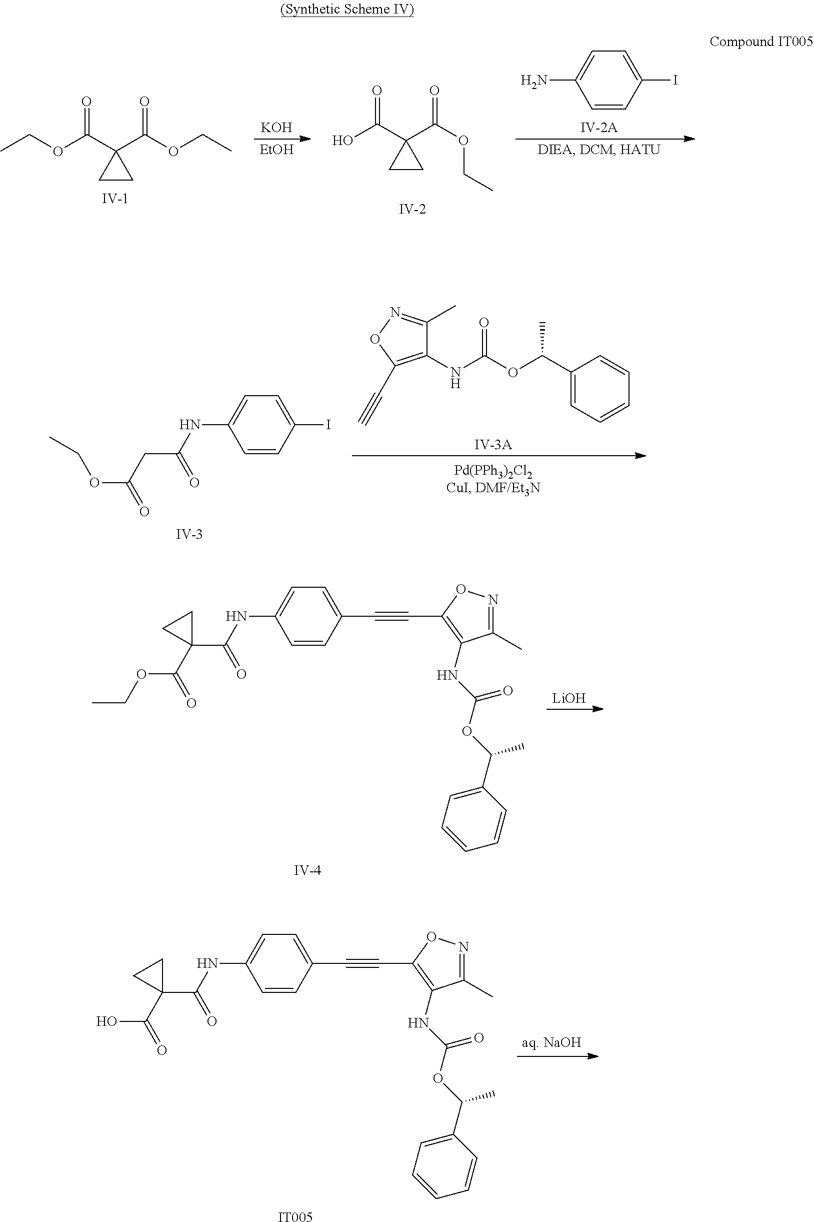

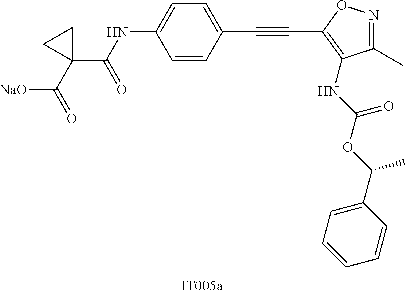

KOH (2.8 g, 50 mmol), was added to a solution of IV-1 (9.3 g, 50 mmol) in 200 mL EtOH. The reaction mixture was stirred at rt overnight. After concentrated under reduced pressure, the residue was re-dissolved in 50 mL of NaHCO₃ solution (w/w=5%) and extracted with DCM. The aqueous layer was separated, and adjusted pH to 2 with 1N HCl, and extracted with EtOAc. The combined organic layer was dried and concentrated to afford IV-2 (6.0 g, yield 79%), which was used to next step directly.

IV-2A (2.19 g, 10 mmol) was added to a mixture of IV-2 (1.58 g, 10 mmol) and HATU (4.56 g, 12 mmol) in 20 mL of DCM. The reaction mixture was stirred at rt overnight. Then water (15 mL) was added and extracted with DCM. The organic layer was separated, dried and concentrated. The residue was purified by column (PE/EA=10/1) to afford IV-3 (1.2 g, yield 33.4%).

IV-4, IT005, and IT005a were prepared following the similar procedure described in the preparation of I-6, IT001 and IT001a. IT005: MS (ESI) m/z (M+H)⁺474.1. IT005a: ¹H NMR (DMSO-d₆, 400 MHz): δ 7.23 (d, J=8.4 Hz, 2H), 7.28-7.41 (m, 7H), 5.81-5.83 (q, 1H), 2.18 (m, 3H), 1.57 (d, J=6.4 Hz, 3H), 1.47-1.49 (m, 2H), 1.40-1.43 (m, 2H). MS (ESI) m/z (M+H)⁺474.1.

### Example 2-B

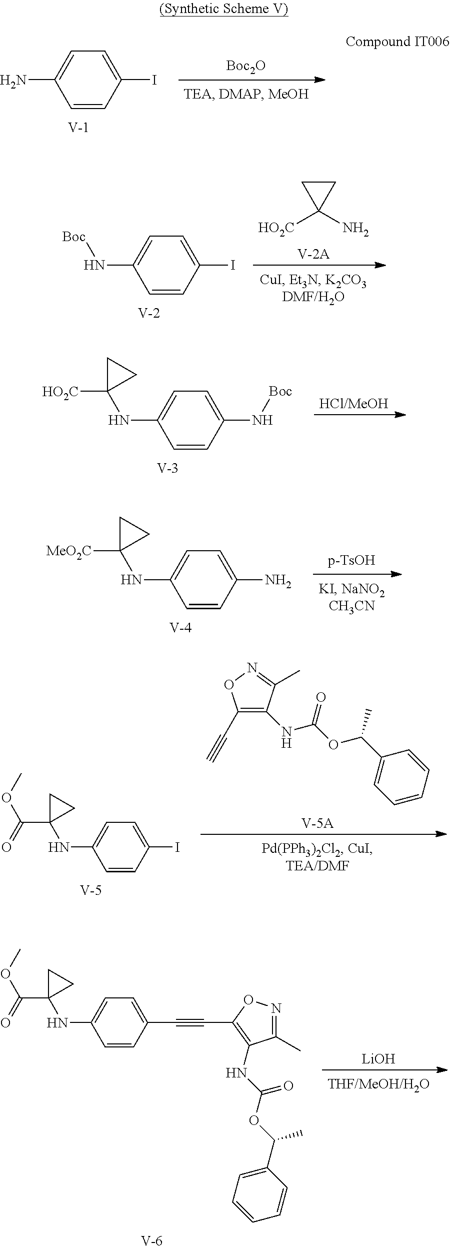

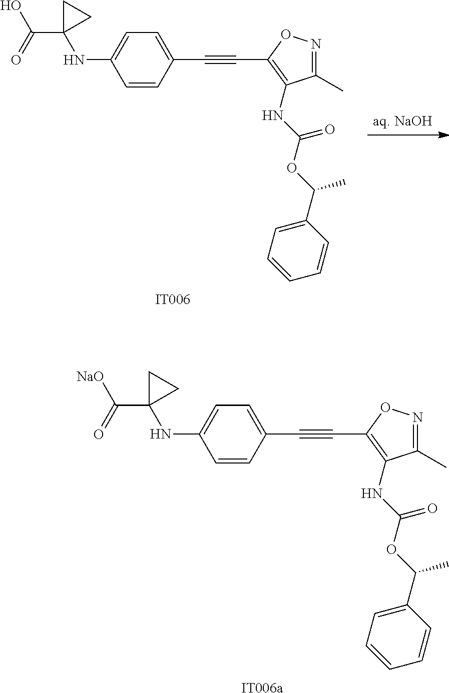

To a solution of V-1 (10 g, 45.66 mmol), TEA (9.22 g, 91.23 mmol) and DMAP (50 mg) in MeOH (100 mL) was added di-tert-butyl dicarbonate (19.8 g, 50.2 mmol). The mixture was heated to 50° C. overnight. After completion of the reaction, the mixture was concentrated, the residue was purified by column chromatography (PE/EA=10/1) to afford V-2 (8.47 g, yield 58.2%).

To a solution of V-2 (4 g, 12.53 mmol) and V-2A (1.9 g, 18.80 mmol) in DMF/H₂O (40 mL, v/v=3/1) was added K₂CO₃ (5.2 g, 37.6 mmol), Et₃N (0.18 mL, 1.25 mmol) and CuI (0.48 g, 2.51 mmol). The reaction mixture was heated to 110° C. and stirred overnight. After completion of the reaction, the mixture was diluted with H₂O, extracted with EtOAc, the combined organic layer was washed with brine, dried and concentrated to afford V-3 (3.9 g, crude), which was used to next step directly.

A mixture of crude V-3 (3.9 g) in 4 N HCl in methanol (60 mL) was heated to reflux for 4 hours. The mixture was concentrated. The residue was dissolved in ethyl acetate, washed with saturated NaHCO₃, dried and concentrated. The residue was purified by flash column chromatography on silica gel (PE:EA=2/1) to afford V-4 (0.8 g, yield 31% over two steps).

To a stirred solution of p-TsOH.H₂O (2.2 g, 11.64 mmoL) in CH₃CN (15 mL) was added V-4 (800 mg, 3.88 mmol. The resulting suspension of amine salt was cooled to 5° C. and a solution of NaNO₂ (535 mg, 7.76 mmol) and KI (1.61 mg, 9.70 mmol) in H₂O was added dropwise. The mixture was stirred overnight at rt. The mixture was concentrated in vacuum. The residue was partitioned between ethyl acetate and saturated NaHSO₃. The organic layer was washed with brine, dried and concentrated. The residue was purified by flash column chromatography on silica gel (PE:EA=8/1) to afford V-5 (300 mg, yield 25%).

V-6, IT006, and IT006a were prepared following the similar procedure described in the preparation of I-6, IT001 and IT001a. IT006a: ¹H NMR (400 MHz, DMSO-d₆): δ 9.30 (br, 1H), 7.31-7.38 (m, 5H), 7.14 (d, J=8.8 Hz, 2H), 6.82 (s, 1H), 6.58 (d, J=8.8 Hz, 2H), 5.77 (q, J=6.4 Hz, 1H), 2.12 (s, 3H), 1.52 (d, J=6.48 Hz, 3H), 1.21-1.24 (m, 2H), 0.58-0.59 (m, 2H). MS (ESI) ink (M+H)⁺446.1.

### Example 3-A

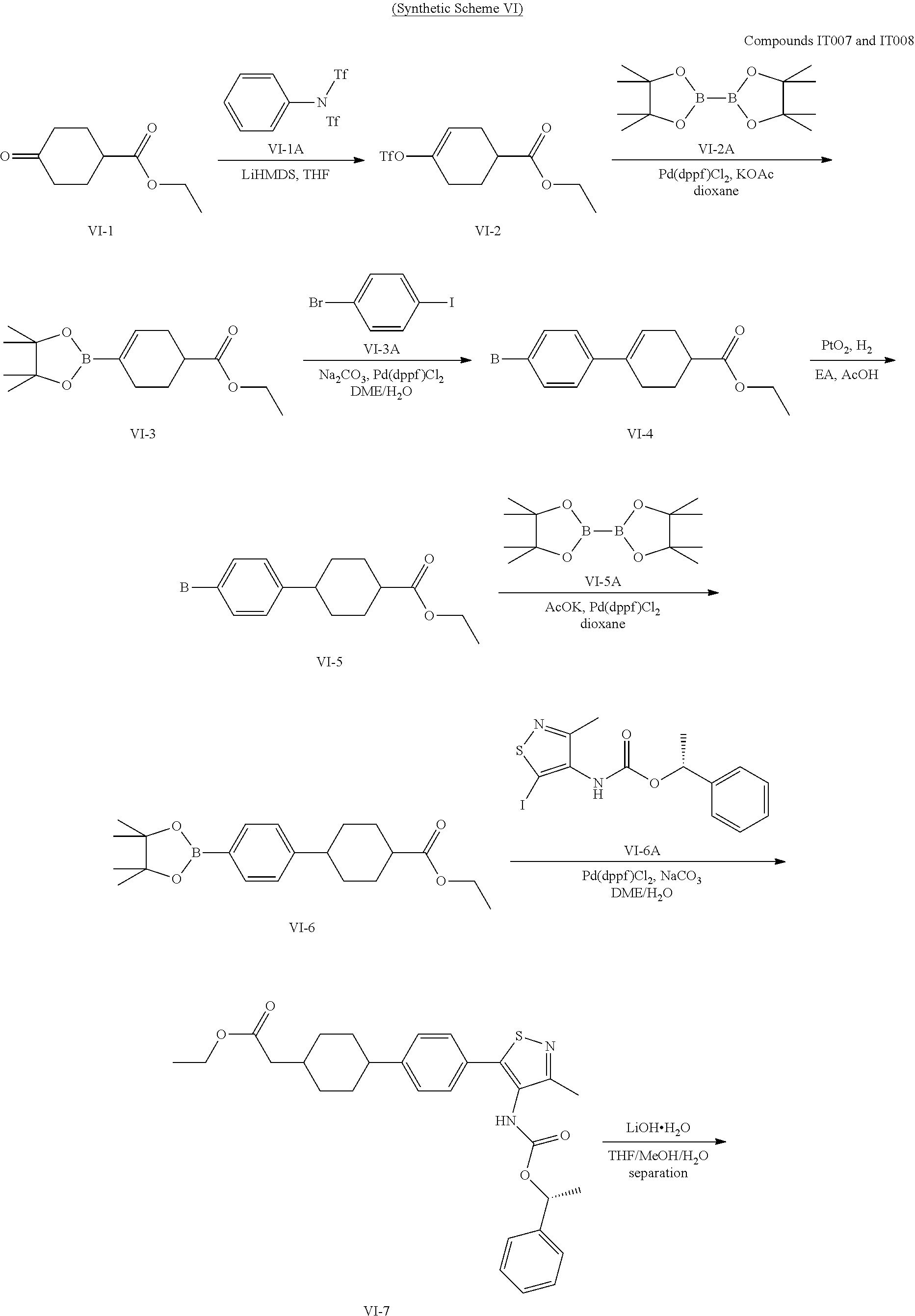

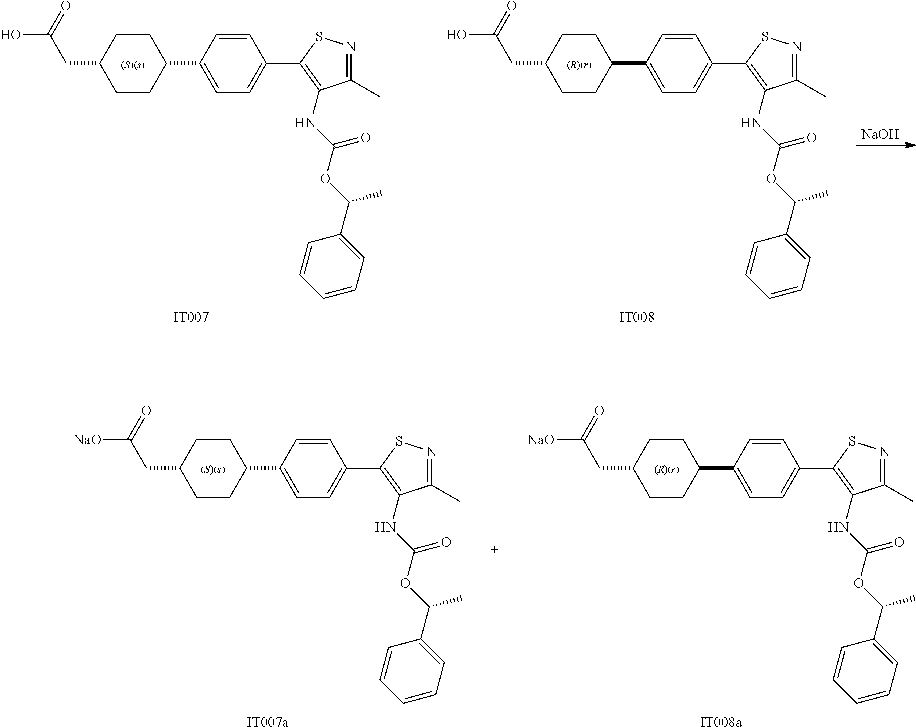

To a stirred solution LiHMDS (23.5 g, 141 mmol) in THF (400 mL) was added VI-1 (20 g, 128.2 mmol) at −78° C. After 30 min VI-1A (50 g, 141 mmol) was added to the dark brown solution. After stirred for 30 min at −78° C., the mixture was allowed to warm to rt. The mixture was diluted with EA (500 mL×3) washed with aq NaHCO₃ (300 mL), and the combined organic layer was washed with brine, dried over Na₂SO₄, and concentrated. The residue was purified by column chromatography on silica gel (PE:EA=5:1) to afford VI-2 (20 g, yield: 54.2%).

The mixture of VI-2 (3 g, 10.4 mmol), VI-2A (3.17 g 12.5 mmol), KOAc (2.0 g, 20.8 mmol) and Pd(dppf)Cl₂ (0.3 g) in dioxane (60 mL) was heated to reflux under nitrogen overnight. After concentrated under reduced pressure, the residue was partitioned between H₂O (60 mL) and DCM (60 mL), the aqueous phase was extracted with DCM, and the combined organic layer was washed with brine, dried over Na₂SO₄, and concentrated. The residue was purified by column chromatography on silica gel (PE:EA=5:1) to afford VI-3 (1.1 g, yield: 37.9%).

The mixture of VI-3A (3.0 g, 10.7 mmol), VI-3 (3 g, 10.7 mmol), Na₂CO₃ (2.7 g, 21.4 mmol) and Pd(dppf)Cl₂ in DME/H₂O (90 mL, v/v=3:1) was heated to reflux under nitrogen overnight. After concentrated under reduced pressure, the mixture was partitioned between H₂O (60 mL) and DCM (60 mL), the aqueous phase was extracted with DCM, and the combined organic layer was washed with brine, dried over Na₂SO₄, concentrated. The residue was purified by column chromatography on silica gel (PE:EA=10:1) to afford VI-4 (1.5 g, yield: 45.45%).

The mixture of VI-4 (1.8 g, 5.8 mmol), AcOH (40 mg, 0.58 mmol) and PtO₂ (180 mg) in EtOAc (20 mL) was stirred at rt under H₂ (45 psi) overnight. After concentrated, the residue was partitioned between H₂O (30 mL) and DCM (30 mL), the aqueous phase was extracted with DCM, and the combined organic layer was washed with aq. NaHCO₃, brine, dried over Na₂SO₄, and concentrated. The residue was purified by column chromatography on silica gel to (PE:EA=7:1) afford VI-5 (1.1 g, yield: 60.77%).

VI-6 was prepared following the similar procedure for the synthesis of VI-3.

VI-7, IT007, IT008, IT007a and IT008a were prepared following the similar procedure described in the preparation of I-6, IT001 and IT001a. IT007 and IT008 were obtained by chiral separation: MS (ESI) m/z (M+H)⁺465.2. IT007a: ¹H NMR (Methanol-d₄, 400 MHz) δ 57.27-7.40 (m, 9H), 5.69-5.79 (m, 1H), 2.54-2.61 (m, 1H), 2.52 (br, 1H), 2.27-2.31 (m, 5H), 1.71-1.85 (m, 2H), 1.57-1.69 (m, 7H). MS (ESI) m/z (M+H)⁺465.2. IT008a: ¹H NMR (Methanol-d₄, 400 MHz): δ7.26-7.41 (m, 9H), 5.69-5.80 (m, 1H), 2.55-2.61 (m, 1H), 2.32 (s, 3H), 2.20-2.25 (m, 1H), 2.07-2.10 (m, 2H), 1.91-1.94 (m, 2H), 1.51-1.65 (m, 7H). MS (ESI) m/z (M+H)⁺465.2.

### Example 3-B

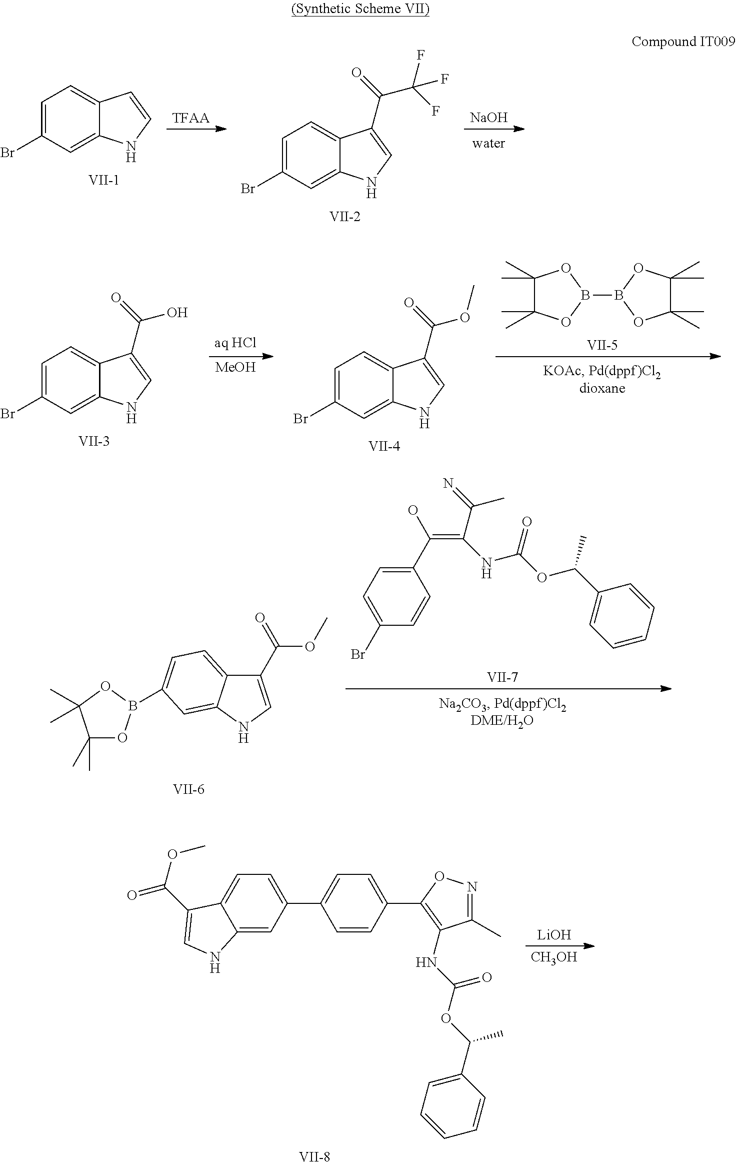

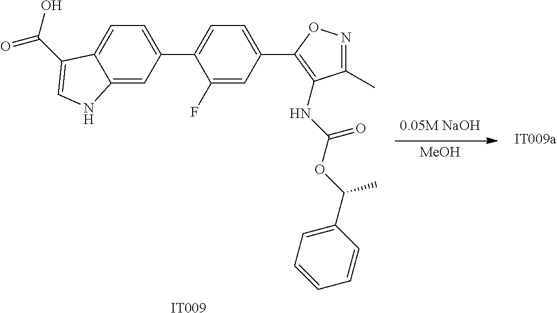 [SMILES: "MeOH" ; Cc1noc(-c2ccc(-c3ccc4c(C(=O)O)c[nH]c4c3)c(F)c2)c1NC(=O)O[C@H](C)c1ccccc1 ; "IT009" ; "IT009a"]

To the solution of VII-1 (3.9 g, 0.02 mol) in dry DMF (40 mL) was added TFAA (4.8 g, 0.028 mol) by dropwise at 0° C. and the reaction mixture was stirred for 3 hs at the same temperature. The solution was poured into water and the appeared solid was collected by filtration. The solid was washed with DCM to afford VII-2 (4.5 g, yield 77%) as a yellow solid.

To a stirred solution of VII-2 (1.7 g, 5.8 mmol) and NaOH (2.3 g, 58 mmol) in THF/water=1:1 (40 mL) was heated to reflux and stirred for 24 hours. The solvent was removed and the residue was added 2M HCl to adjust pH=2, the solid was collected and dried to give VII-3 (0.7 g, yield 50%) as a yellow solid.

To a stirred solution of VII-3 (0.86 g, 3.6 mmol) in MeOH (30 mL) was added aq. HCl (0.5 mL) under nitrogen. After the addition, the solution was heated to reflux under nitrogen for 2 hours. The solvent was removed by reduced pressure and the residue was added sat. NaHCO₃ to adjust to pH=9 and the solution was extracted with DCM, the combine organic layer was dried and concentrated in vacuum to afford VII-4 (0.71 g, 78%) as a yellow solid which was used for next step directly.

VII-6 and VII-8 were obtained following the similar procedure as described for the preparation of VI-6 and VI-7.

IT009 and IT009a were prepared following the similar procedure described in the preparation of IT001 and IT001a. IT009: MS (ESI) m/z (M+Na)⁺482.1. IT009a: ¹H NMR (Methanol-d₄, 400 MHz): δ8.29 (d, J=8.4 Hz, 1H), 7.65-7.81 (m, 6H), 7.32-7.44 (m, 6H), 5.80-5.82 (q, 1H), 2.18 (s, 3H), 1.56 (d, J=6.4 Hz, 3H). MS (ESI) m/z (M+Na)⁺482.1.

### Example 3-C

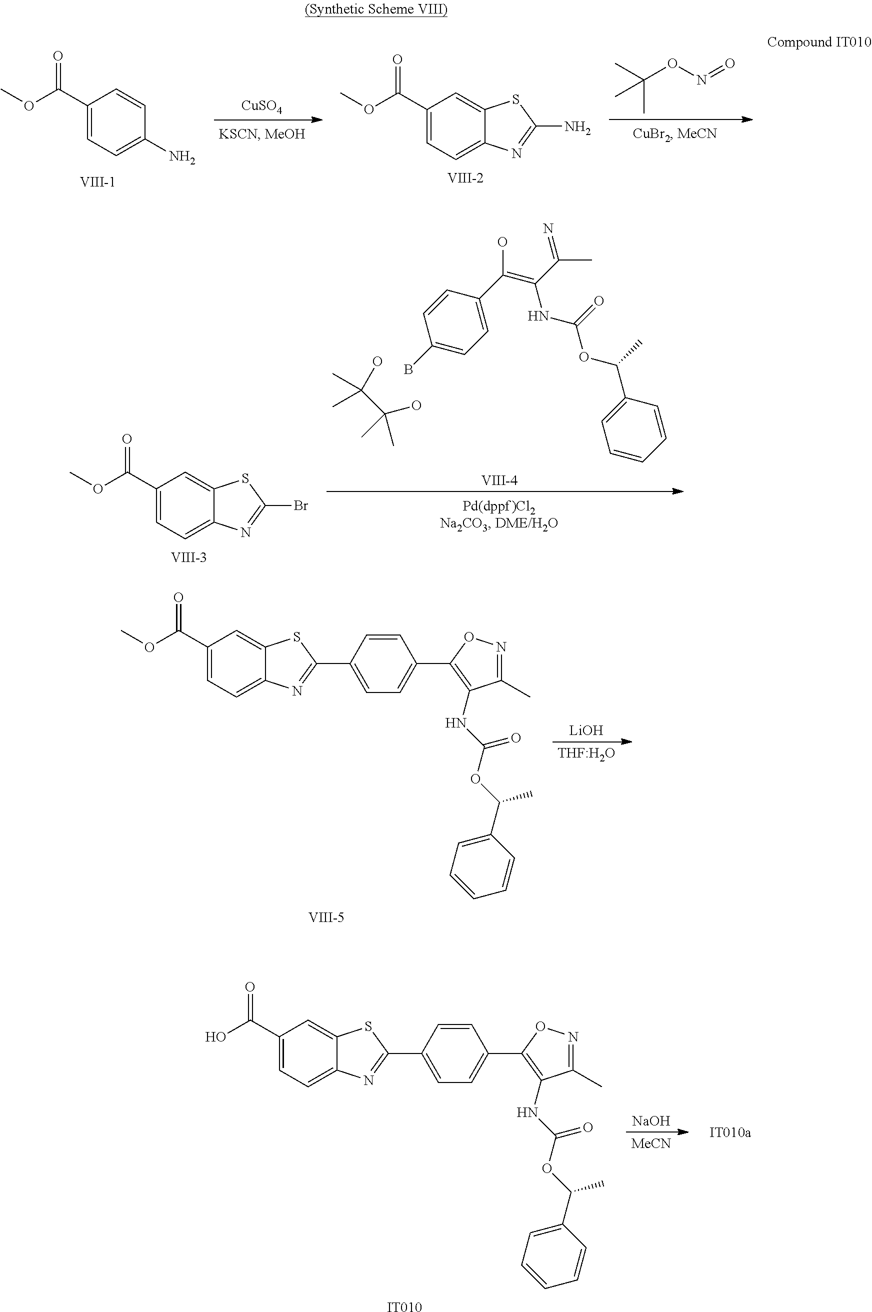

To a solution of VIII-1 (10 g, 66 mmol) in MeOH (100 mL) was added KSCN (51.2 g, 0.53 mol) and CuSO₄ (38.4 g, 0.24 mol). The reaction mixture was heated to 80° C. overnight. The mixture was diluted with EtOAc, washed with brine, dried over Na₂SO₄, filtered and concentrated. The residue was purified by column chromatography (PE:EA=2:1) to give VIII-2 (5 g, yield: 36%).

To a stirred mixture of VIII-2 (600 mg, 2.28 mmol) and CuBr₂ (775 mg, 3.46 mmol) in MeCN (9 mL) was added tert-butyl nitrite (445 mg, 4.32 mmol). The reaction mixture was stirred at rt overnight. The mixture was diluted with EtOAc (40 mL), washed with water and brine, dried over Na₂SO₄, filtered and concentrated. The residue was purified by column (PE:EA=5:1) to give VIII-3 (140 mg, yield: 18%).

To a stirred mixture of VIII-3 (200 mg, 0.738 mmol), VIII-4 (400 mg, 0.88 mmol), and Na₂CO₃ (233 mg, 2.198 mmol) in DME (6 mL) and H₂O (2 mL) was added Pd(dppf)Cl₂ (53.9 mg, 0.0738 mmol). The reaction mixture was flushed with nitrogen and heated to 80° C. overnight. The mixture was diluted with EtOAc (40 mL), washed with water and brine, dried over Na₂SO₄, filtered and concentrated. The residue was purified by column chromatography (PE:EA=10:1) to give VIII-5 (50 mg, yield: 13.15%). MS (ESI) m/z (M+H)⁺514.1.

IT010 and IT010a were prepared following the similar procedure described in the preparation of IT001 and IT001a. IT010: MS (ESI) m/z (M+H)⁺500.1. IT010a: ¹HNMR (DMSO-d₆, 400 MHz) δ 8.58 (s, 1H), 8.12-8.17 (m, 3H), 8.01 (d, J=8.8 Hz, 1H), 7.88 (d, J=7.6 Hz, 2H), 7.36-7.45 (m, 5H), 5.81-5.83 (m, 1H), 2.20 (s, 3H), 1.63 (d, J=6.0 Hz, 3H), MS (ESI) ink (M+H)⁺500.1.

### Example 4-A

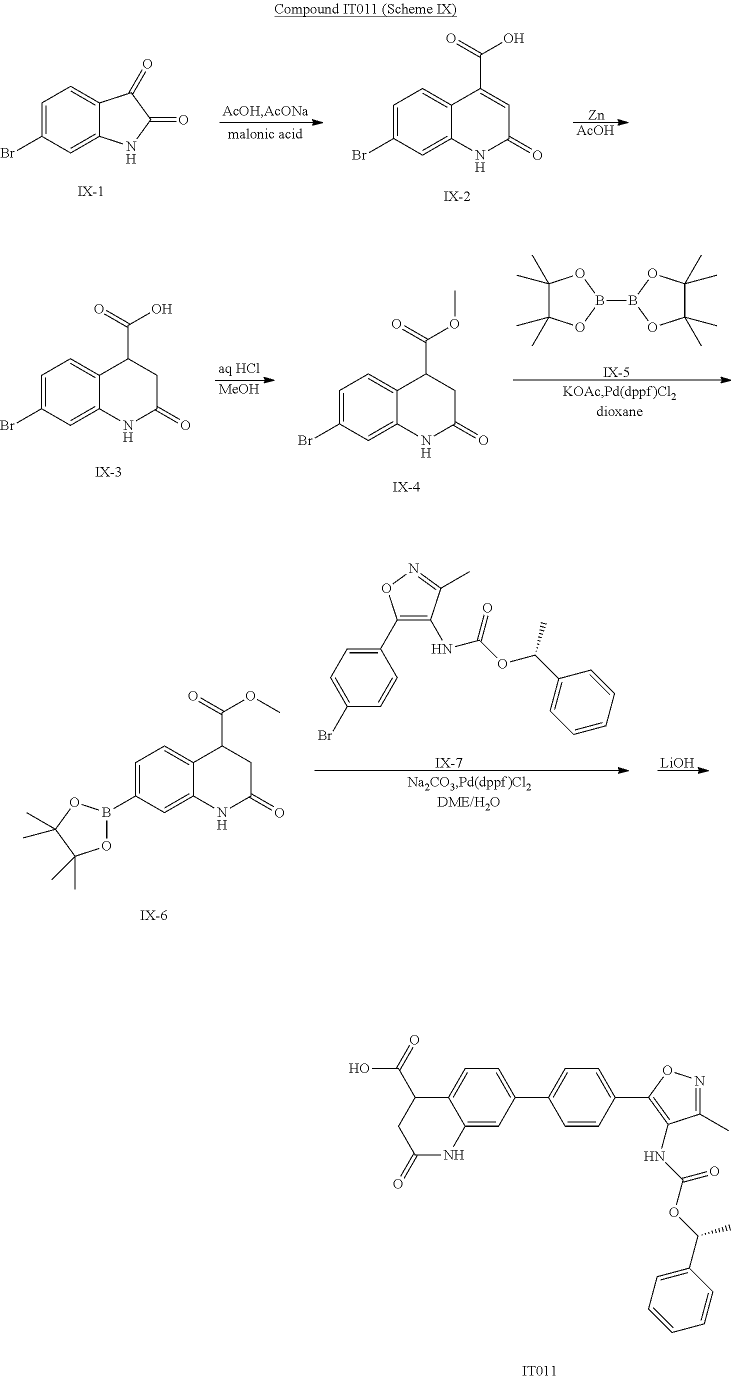

The mixture of IX-1 (6.5 g, 28.7 mmol), malonic acid (3.3 g, 31.7 mmol), NaOAc (2.95 g, 36 mmol) in AcOH (60 mL) were stirred at rt. After 6 hrs, NaOAc (2.95 g, 36 mmol) was added additional, then refluxed overnight. After cooling, the mixture was filtered and the filtrate was washed with water and EtOAc, then dried under reduced pressure to afford IX-2 (5 g, yield 66%) as a brown oil, which was used for next step directly.

The solution of IX-2 (3 g, 11 mmol) and Zn (6 g, 88 mmol) in AcOH (40 mL) was heated to reflux and stirred for 24 hrs. The reaction was filtered and the filtrate was concentrated, the residue was added Sat.NaHCO₃ to adjust pH=9 and extracted with DCM, the aqueous layer was added aq. HCl to adjust pH=5. The solid was collected to afford IX-3 (1 g, yield 33%) as a brown solid.

To a stirred solution of IX-3 (1.05 g, 3.7 mmol) in MeOH (30 mL) was added aq HCl (0.5 mL) under nitrogen. After the addition, the solution was heated to reflux under nitrogen for 2 hrs. The solvent was removed by reduced pressure. The residue was added Sat.NaHCO₃ (10 mL) to adjust pH=9, extracted with EtOAc, the combine organic layers was dried over NaSO₄, concentrated in vacuum to afford crude IX-4 (0.9 g, yield 81%) as a yellow solid, which was used for next step directly.

IX-6 was prepared following the similar procedure described in the preparation of VI-3 as a brown solid. IT011 was prepared following the similar procedure described in the preparation of VIII-5. ¹H NMR (Methanol-d₄, 400 MHz): δ7.82 (d, J=8.0 Hz 2H), 7.68 (d, J=7.6 Hz, 2H), 7.13-7.47 (m, 8H), 5.85 (m, 1H), 4.04 (t, 1H), 2.92 (m, 1H), 2.80-2.89 (m, 2H), 2.20 (s, 3H), 1.63 (d, J=5.6 Hz, 3H). The sodium salt IT011a was prepared following the similar procedure described in the preparation of IT001a. ¹H NMR (Methanol-d₄, 400 MHz): δ 7.78 (d, J=7.6 Hz, 2H), 7.64 (d, J=7.8 Hz, 2H), 7.13-7.47 (m, 8H), 5.81 (m, 1H), 3.79 (t, J=6.4 Hz, 1H), 2.92 (m, 1H), 2.65-2.73 (m, 1H), 2.18 (s, 3H), 1.61 (d, J=5.6 Hz, 3H). MS (ESI) m/z (M+Na)⁺512.1.

### Example 4-B

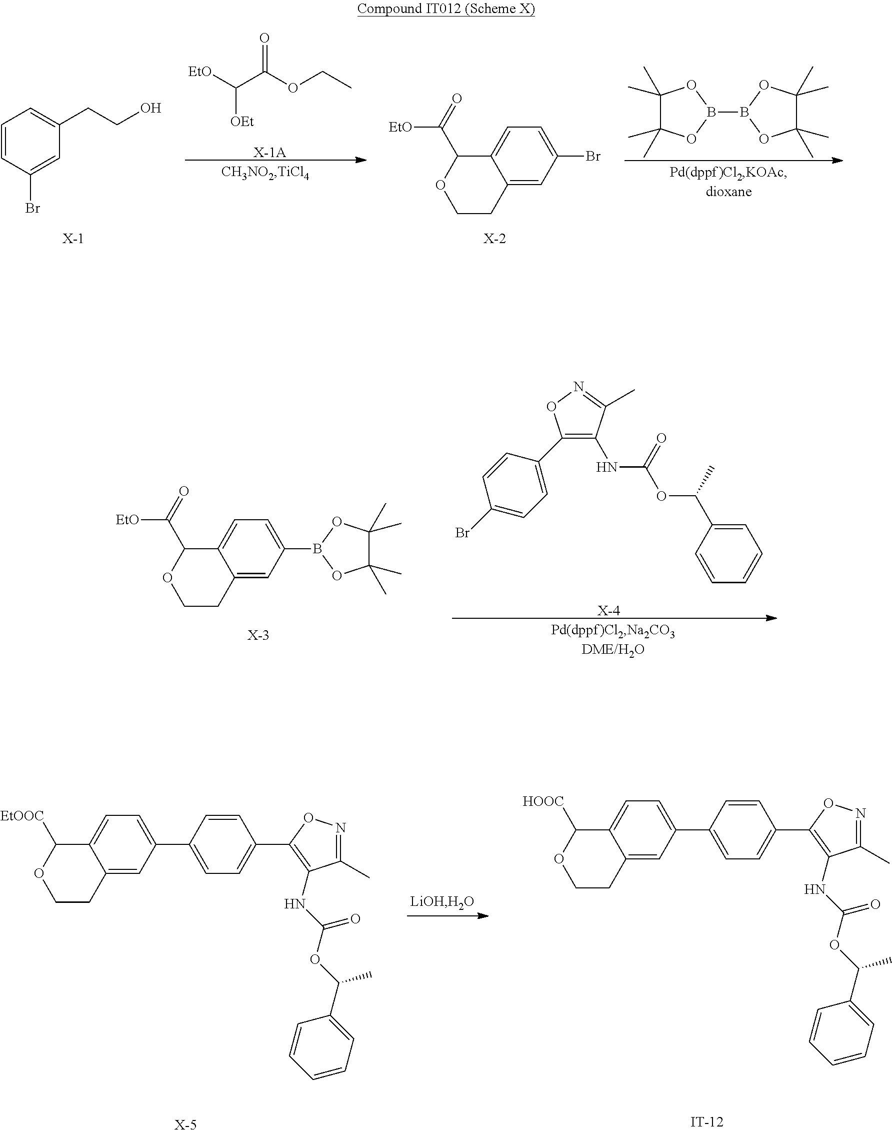

TiCl₄ (7.48 g, 40 mmol) was added over a period of 10 min to an ice-cooled mixture of X-1 (2 g, 10 mmol) and X-1A (2.12 g, 12 mmol) in CH₃NO₂ (20 mL). The solution was allowed to stir at rt for 12 hrs. Then the mixture was poured into the HCl (aq. 1N) and extracted with DCM, dried over Na₂SO₄, concentrated in vacuo. The residue was purified by column chromatography to afford X-2 (0.5 g, yield 95%).

X-3 was prepared following the similar procedure described in the preparation of VI-3 with 67% yield. X-5 was prepared following the similar procedure described in the preparation of VIII-5 with 32% yield.

IT012 and its sodium salt IT012a were prepared following the similar procedure described in the preparation of IT001 and IT001a. IT012: MS (ESI) m/z (M+H)⁺499.2. IT012a: ¹H NMR (DMSO-d₆, 400 MHz): δ 7.75-7.78 (m, 2H), 7.73-7.74 (m, 3H), 7.38-7.42 (m, 4H), 7.31 (s, 1H), 5.74-5.76 (m, 1H,), 4.78 (s, 1H), 4.38-4.41 (m, 1H), 3.71-3.74 (m, 1H), 2.76-2.79 (m, 2H), 2.11 (s, 3H), 1.50 (s, 3H). MS (ESI) m/z (M+H)⁺499.2.

### Example 5

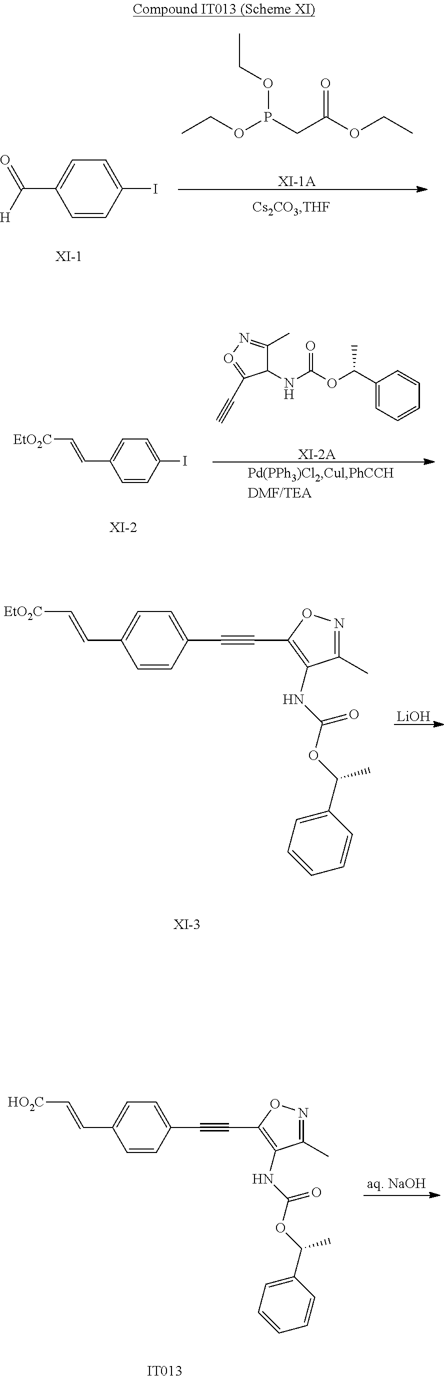

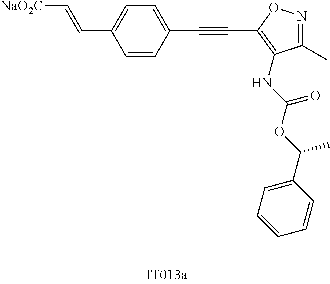

A solution of XI-1 (100 mg, 0.43 mmol), XI-1A (97 mg, 0.43 mmol) and Cs₂CO₃ (210 mg, 0.64 mmol) in 2 mL of THF was stirred overnight at rt. The mixture was treated with H₂O, and extracted with EtOAc. The combined organic layer was washed with brine, dried and concentrated. The residue was purified by prep-TLC (PE) to afford XI-2 (120 mg, yield 93.0%).

To a solution of XI-2 (130 mg, 0.43 mmol) in 2 mL of DMF/TEA (v/v=3/1), which was degassed by argon, was added Pd(PPh₃)₂Cl₂ (13 mg, 0.019 mmol) and phenyl acetylene (8 uL, 0.071 mmol). Then a solution of XI-2A (85 mg, 0.31 mmol) in 6 mL of DMF/TEA (v/v=3/1) was added dropwise. After stirred for 30 minutes, the mixture was diluted with H₂O, and extracted with EtOAc. The combined organic layer was washed with brine, dried and concentrated. The residue was purified by flash column chromatography over silica gel (PE/EA=6/1) to afford XI-3 (91 mg, yield 66%).

IT013 and its sodium salt IT013a were prepared following the similar procedure described in the preparation of IT001 and IT001a. IT013: ¹H NMR (400 MHz, CDCl₃): δ 7.66-7.72 (m, 3H), 7.51 (d, J=8.0 Hz, 2H), 7.30-7.42 (m, 5H), 6.58 (d, J=16 Hz, 1H), 5.85 (q, 1H), 2.22 (s, 3H), 1.59 (d, J=6.4 Hz, 3H). MS (ESI) m/z (M+H)⁺417.1. IT013a: ¹H NMR (DMSO-d₆, 400 MHz): δ 9.55 (br, 1H), 7.53 (d, J=8.4 Hz, 2H), 7.30-7.41 (m, 7H), 7.05 (d, J=16 Hz, 1H), 6.44 (d, J=16 Hz, 1H), 5.77 (q, 1H), 2.151 (s, 3H), 1.51 (d, J=6.4 Hz, 3H). MS (ESI) m/z (M+H)⁺ 417.1.

### Example 6-A

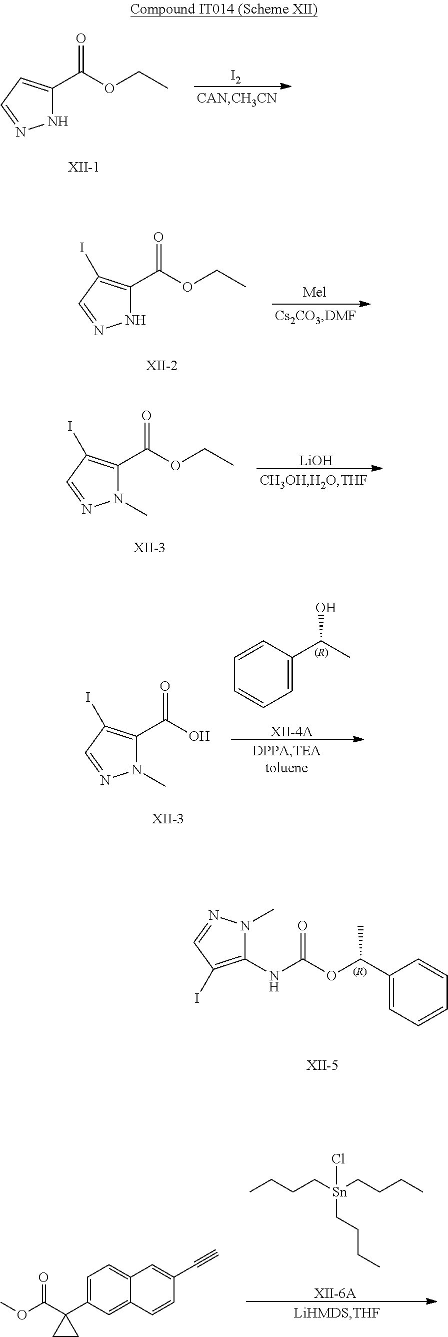

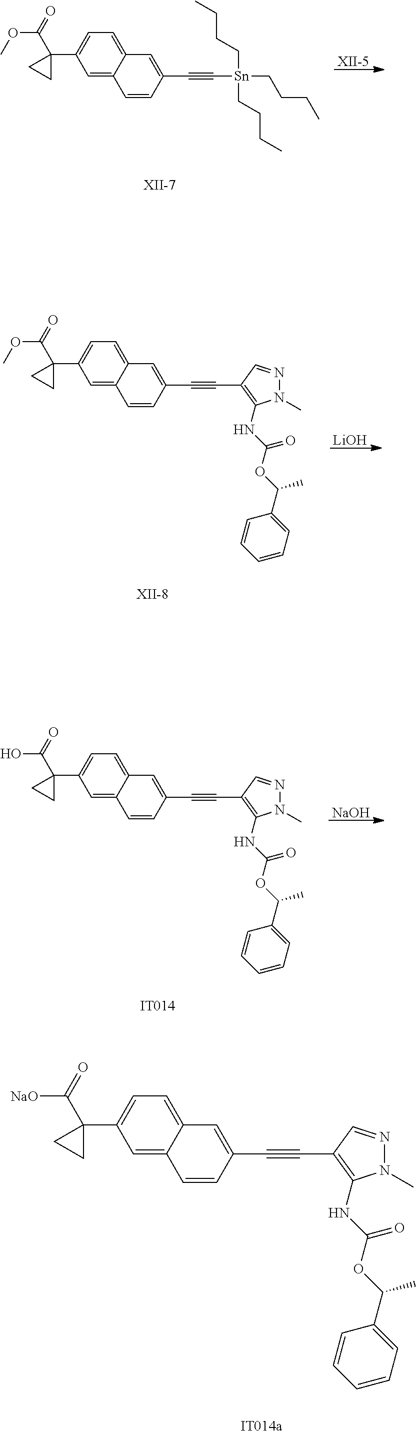

XII-6 was prepared from 2-(6-bromonaphthalen-2-yl)acetonitrile in five-step reactions.

To a solution of XII-1 (10 g, 71.4 mmol) in CH₃CN (182 mL) was added CAN (39.1 g, 71.4 mmol). The mixture was stirred at 25° C. for 15 min. Then I₂ (18 g, 71.4 mmol) was added. The mixture was stirred at 25° C. for 12 h. Then the mixture was quenched with 5% cold aq. NaHSO₃, until the solution turned into light yellow. The solid was filtered. The filtrate was extracted with EtOAc. The organics were collected, dried with Na₂SO₄, filtered, and concentrated. The residue was purified by column (PE:EA=3:1) to afford XII-2 (7.8 g, yield: 41%).

To a solution of XII-2 (8 g, 30.07 mmol) in DMF (150 mL) was added Cs₂CO₃ (29.3 g, 90 mmol) and CH₃I (10.6 g, 75.2 mmol). The mixture was stirred at 25° C. for 12 h. Then the mixture was washed with H₂O, and extracted with EtOAc. The organics were combined, dried with Na₂SO₄, filtered, and concentrated under reduced pressure. The residue was purified by column chromatography (PE:EA=10:1) to afford XII-3 (4 g, yield: 47.5%).

To a stirred solution of XII-3 (700 mg, 2.5 mmol) in 15 mL of MeOH/H₂O/THF (v/v/v=1/1/1) was added lithium hydroxide monohydrate (1.05 mg, 25 mmol). After the addition, the solution was stirred at 25° C. for 2 h. The mixture was concentrated in vacuo and adjusted pH to 4 with HCl (1N). The aqueous phase was extracted with EtOAc. The combined organic layer was washed with brine, dried over Na₂SO₄, and concentrated to afford crude XII-4 (623.0 mg, crude yield 98%), which was used to next step directly.

The mixture of XII-4 (2.1 g, 8.3 mmol), XII-4A (1.2 g, 9.8 mmol), DPPA (2.7 g, 9.8 mmol) and TEA (1.68 g, 16.6 mmol) in toluene (20 mL) was stirred at 80° C. under nitrogen for 2 hrs. Then the mixture was washed with H₂O, and extracted with EtOAc. The organics were combined, dried with Na₂SO₄, filtered, and concentrated under reduced pressure. The residue was purified by column chromatography on silica gel (PE: EA=3:1) to afford XII-5 (2.3 g, yield: 74.4%).

To a mixture of XII-6 (1.0 g, 4.0 mmol) in THF (10 mL) was added LiHMDS (4.8 mL, 4.8 mmol) at −78° C. The reaction mixture was stirred for 1 h at −78° C. and then XII-6A (2.60 g, 4.8 mmol) was added. The reaction mixture was stirred overnight and quenched with satur. NH4Cl (10 mL). The mixture was extracted with EtOAc, and the combined organic layer was washed with brine, dried over Na2SO4, and concentrated. The residue was purified by chromatography on silica gel (PE/EA=4:1) to afford XII-7 (410 mg, yield: 18.6%).

To a mixture of XII-7 (335.6 mg, 0.622 mmol), PPh₃ (18.7 mg, 0.072 mmol) and XII-5 (210 mg, 0.566 mmol) in THF (10 mL) was added Pd(OAc)₂ (7.9 mg, 0.036 mmol) under Ar at rt. The reaction mixture was heated at 50° C. for 2 hrs and then diluted with water. The mixture was extracted with EtOAc, and the combined organic layer was washed with brine, dried over Na₂SO₄, concentrated to get XII-8 (150 mg, crude yield: 49.0%), which was used directly without further purification.

IT014 and its sodium salt IT014a were prepared following the similar procedure described in the preparation of IT001 and IT001a. IT014: MS (ESI) m/z (M+H)⁺480.0. IT014a: ¹H NMR (DMSO-d₆, 400 MHz) δ7.85 (s, 1H), 7.63-7.75 (m, 4H), 7.28-7.38 (m, 7H), 5.78-5.80 (q, 1H), 3.61 (s, 3H), 1.50-1.52 (d, J=6.0 Hz, 3H), 1.21 (brs, 2H), 0.77 (brs, 2H). MS (ESI) m/z (M+H)⁺480.0.

IT015 was prepared following the similar synthetic route for the preparation of IT014 using ethyl 1-cyclopropyl-4-iodo-1H-pyrazole-5-carboxylate (XII-3A) in place of XII-3. Preparation of XII-3A: To a solution of XII-2 (6.8 g, 25.5 mmol) in 1,4-dioxane (200 mL) was added Cu(OAc)₂ (3.9 g, 21.4 mmol), Cs₂CO₃ (20.7 g, 63.5 mmol), DMAP (12.5 g, 102.5 mmol) and cyclopropylboronic acid (4.39 g, 51.04 mmol). The mixture was stirred at 50° C. for 12 h. The solvent was removed under reduced pressure. Then the mixture was washed with H₂O, extracted with EtOAc. The organics were combined, dried with Na₂SO₄, filtered, and concentrated. The residue was purified by column (PE:EA=30:1) to afford XII-3A (2.2 g, yield: 28.2%). IT015: MS (ESI) m/z (M+H)⁺506.2.

Sodium salt IT015a was prepared following the similar procedure described in the preparation of IT001a. ¹H NMR (DMSO-d₆, 400 MHz): δ7.86 (s, 1H), 7.76 (d, J=8.4 Hz, 1H), 7.65-7.76 (m, 3H), 7.52 (d, J=8.4 Hz, 1H), 7.29-7.39 (m, 6H), 5.79-5.83 (m, 1H), 3.39-3.41 (m, 1H), 1.52 (d, J=5.2 Hz, 3H), 1.20-1.21 (br, 2H), 0.89-0.90 (m, 4H), 0.75 (br, 2H). MS (ESI) m/z (M+H)⁺506.2.

IT016 was prepared following the similar synthetic route for the preparation of IT014 using ethyl 1-ethyl-4-iodo-1H-pyrazole-5-carboxylate (XII-3B) in place of XII-3. XII-3B was prepared following the similar procedure for the synthesis of XII-3 using C₂H₅₁ in place of CH₃I. IT016: MS (ESI) m/z (M+H)⁺494.2.

Sodium salt IT016a was prepared following the similar procedure described in the preparation of IT001a. ¹H NMR (DMSO-d₆, 400 MHz) δ: 7.84 (s, 1H), 7.76 (d, J=8.4 Hz, 1H), 7.63-7.67 (m, 3H), 7.53 (d, J=8.4 Hz, 1H), 7.21-7.37 (m, 6H), 5.78-5.80 (q, 1H), 3.91-3.96 (q, 2H), 1.50 (d, J=6.0 Hz, 3H), 1.23-1.27 (m, 5H), 0.77 (br, 2H). MS (ESI) m/z (M+H)⁺494.2.

### Example 6-B

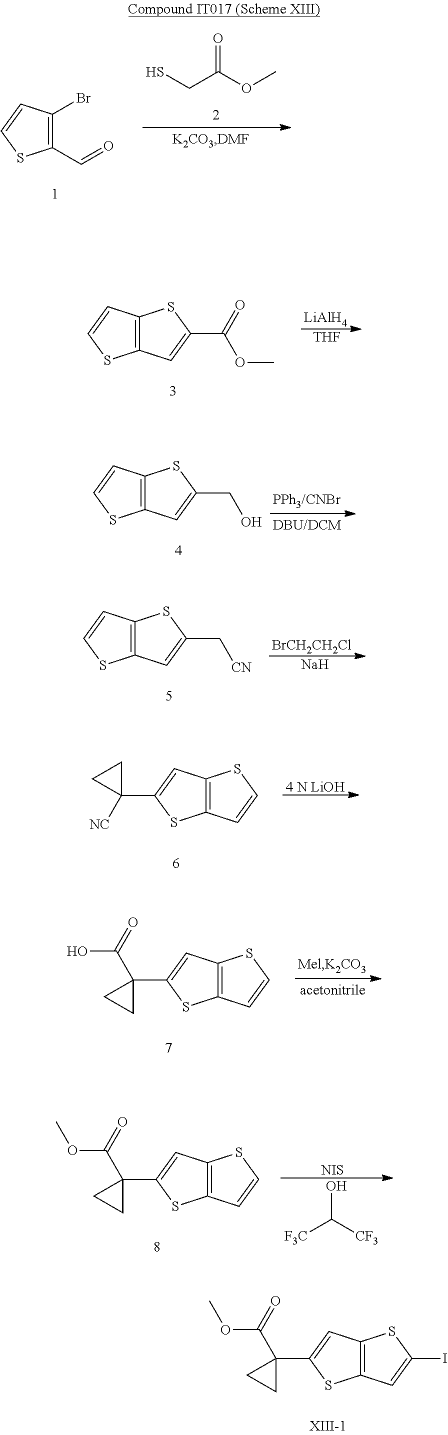

To a mixture of compound 1 (350 g, 1.83 mol) and K₂CO₃ (1000 g, 7.33 mol) in DMF (4000 mL) was added compound 2 (195 g, 1.83 mol) at rt. The resultant mixture was stirred at 70° C. for 5 hs. After cooled to rt, the mixture was poured into ice-water and solids were precipitated out which was obtained by filtration and dried in vacuo at 50° C. to give compound 3 (300 g, 82.6%) as a white solid.

Under nitrogen, compound 3 (300 g, 1.52 mol) dissolved in anhydrous THF (2500 mL) was added dropwise to a mixture of LiAlH₄ (75 g, 1.98 mol) in anhydrous THF (1500 mL) at 0° C. After the addition, the mixture was stirred at rt for 2 hs and compound 3 was consumed completely. Cooled to 0° C., water (75 mL) was added dropwise followed by the addition of 10% NaOH aq. (125 mL) dropwise. The mixture was filtered and the cake was washed with DCM several times. The filtration was concentrated under reduced pressure to give compound 4 (258 g, 77.6%) as a white solid.

Under nitrogen, PPh₃ (415.2 g, 1.58 mol) dissolved in anhydrous DCM (1000 mL) was added to BrCN (183 g, 1.73 mol) at 0° C., followed by the addition of compound 4 (245 g, 1.44 mol) dissolved in anhydrous DCM (3000 mL). The resultant solution was stirred at rt until compound 4 was consumed completely and then the solution was cooled to 0° C. and DBU (285 g, 1.87 mol) was added dropwise. After the addition, the solution was stirred at rt for 16 hs. The solvent was removed under reduced pressure to give the residue which was purified by silica gel column chromatography (PE/EA=20:1) to afford compound 5 (150 g, 58.15%) as a yellow solid.

Under nitrogen, to a mixture of NaH (60%, 56 g, 1.4 mol) in anhydrous THF (500 mL) was added a solution of compound 5 (100 g, 0.56 mol) in anhydrous THF (500 mL) dropwise at 0° C. The mixture was stirred at 0° C. for 1 h, followed by the addition of 1-bromo-2-chloroethane (120 g, 0.84 mol) at 0° C. and the mixture was stirred at rt for 5 hs. Quenched with water, the mixture was diluted with water, extracted with EA, dried over Na2SO4, filtered and concentrated to give the residue which was purified by silica gel column chromatography (PE/EA=20:1) to afford compound 6 (203 g, 89.5%) as a yellow solid.

To a solution of LiOH in water (4N, 300 mL) was added compound 6 (60 g, 0.292 mmol) and the mixture was heated to reflux for 16 hs. After cooled to rt, the solution was extracted with DCM twice and the aqueous phase was acidified to pH˜2 with conc. HCl. The precipitate was collected by filtration, washed with water and dried in vacuo to give compound 7 (57 g, 82%) as a white solid.

To a mixture of compound 7 (400 g, 1.78 mol) and K₂CO₃ (493 g, 3.57 mol) in acetonitrile (4000 mL) was added CH₃I (304 g, 2.15 mol). The resultant mixture was heated to reflux for 16 hs. After cooled to rt, the mixture was filtered and the filtration was concentrated to give compound 8 (370 g, 87%).

To a solution of compound 8 (220 g, 0.923 mol) and 2,6-dimethylpyridine (99 g, 0.923 mol) in 1,1,1,3,3,3-hexafluoropropan-2-ol (2000 g) was added NIS (229 g, 1.02 mol) at rt. The reaction mixture was stirred at rt overnight. LCMS showed the reaction was completed, and then the mixture was diluted with water and extracted with EtOAc. The combined organic layers were washed with brine, and concentrated under reduced pressure. The residue was triturated with EA to give XIII-1 (310 g, 92%) as a pale solid.

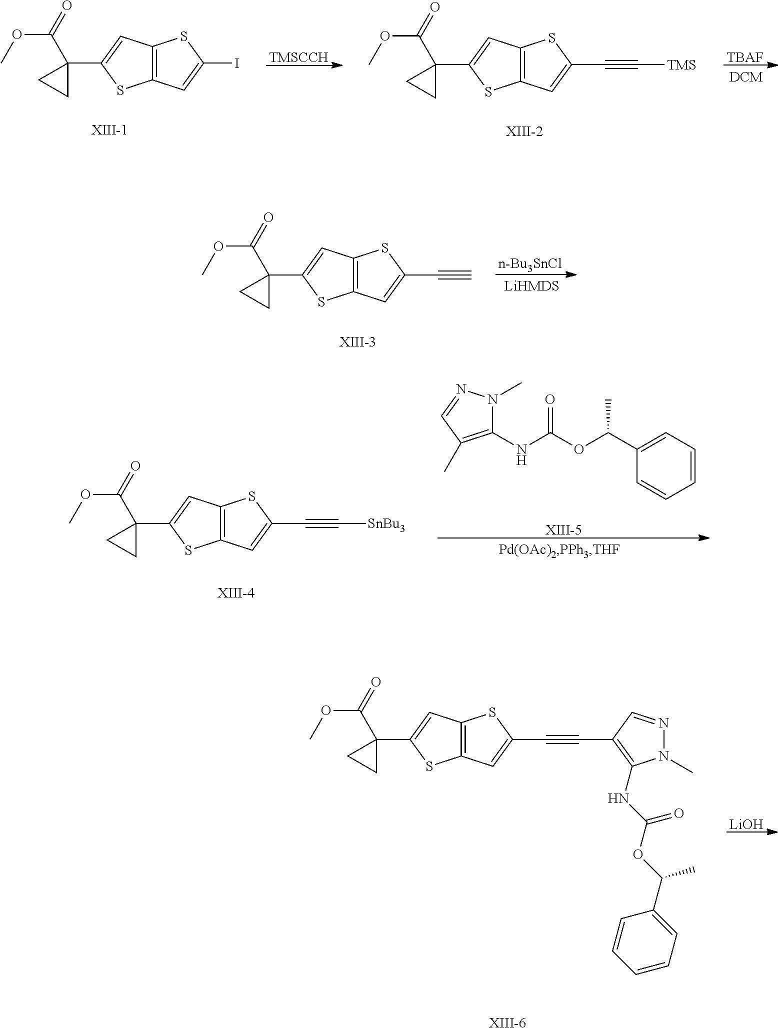

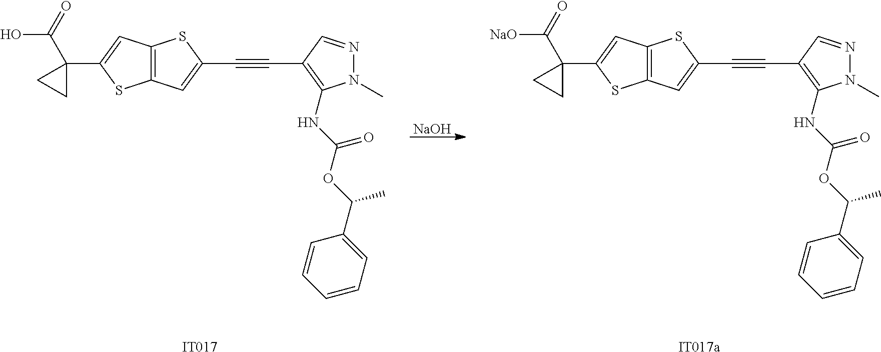

To a mixture of XIII-1 (2.0 g, 5.49 mmol), CuI (25.3 mg, 0.27 mmol) and Pd(PPh)₂Cl₂ (192 mg, 0.27 mmol) in DME/TEA (50 mL, v/v=3:1) was added TMSCCH (1.62 g, 16.48 mmol). The reaction mixture was stirred for 2 h and diluted with water (50 mL). The mixture was extracted with EA, and the combined organic layer was washed with brine, dried over Na₂SO₄, concentrated to afford crude XIII-2 (1.50 g, yield: 81.8%), which was used directly without further purification.

To a mixture of XIII-2 (1.50 g, 4.5 mmol) in DCM (30 mL) was added TBAF (2.70 g, 11.25 mmol). The reaction mixture was stirred for 2 h and diluted with water. The mixture was extracted with DCM, and the combined organic layer was washed with brine, dried over Na₂SO₄, concentrated. The residue was purified by chromatography on silica gel (PE:EA=10:1) to afford XIII-3 (780 mg, yield: 66.1%).

To a mixture of XIII-3 (780 mg, 2.96 mmol) in THF (10 mL) was added LiHMDS (8.8 mL, 8.8 mmol) at −78° C. The reaction mixture was stirred for 1 h at −78° C. and n-Bu₃SnCl (3.0 g, 9.23 mmol) was added. The reaction mixture was stirred for overnight and quenched with sat. NH₄Cl (10 mL). The mixture was extracted with EA, and the combined organic layer was washed with brine, dried over Na₂SO₄, and concentrated to afford crude XIII-4 (1.42 g, yield: 89.3%), which was used directly without further purification.

To a mixture of XIII-4 (334 mg, 0.61 mmol), PPh₃ (17.3 mg, 0.061 mmol) and XIII-5 (225 mg, 0.61 mmol) in THF (10 mL) was added Pd(OAc)₂ (7.3 mg, 0.03 mmol) under Argon at rt. The reaction mixture was heated at 50° C. for 2 h and then diluted with water (20 mL). The mixture was extracted with EtOAc, and the combined organic layer was washed with brine, dried over Na₂SO₄, and concentrated to get crude product, which was purified by prep-HPLC to afford XIII-6 (98 mg, yield: 32.5%).

IT017 and its sodium salt IT017a were prepared following the similar procedure described in the preparation of IT001 and IT001a. IT017a: ¹H NMR (DMSO-d₆, 400 MHz): δ 7.60 (s, 1H), 7.26-7.37 (m, 6H), 6.81 (s, 1H), 5.74-5.76 (q, 1H), 3.57 (s, 3H), 1.48-1.50 (d, J=6.4 Hz, 3H), 1.43 (br, 2H), 0.94 (br, 2H) MS (ESI) m/z (M+H)⁺492.1.

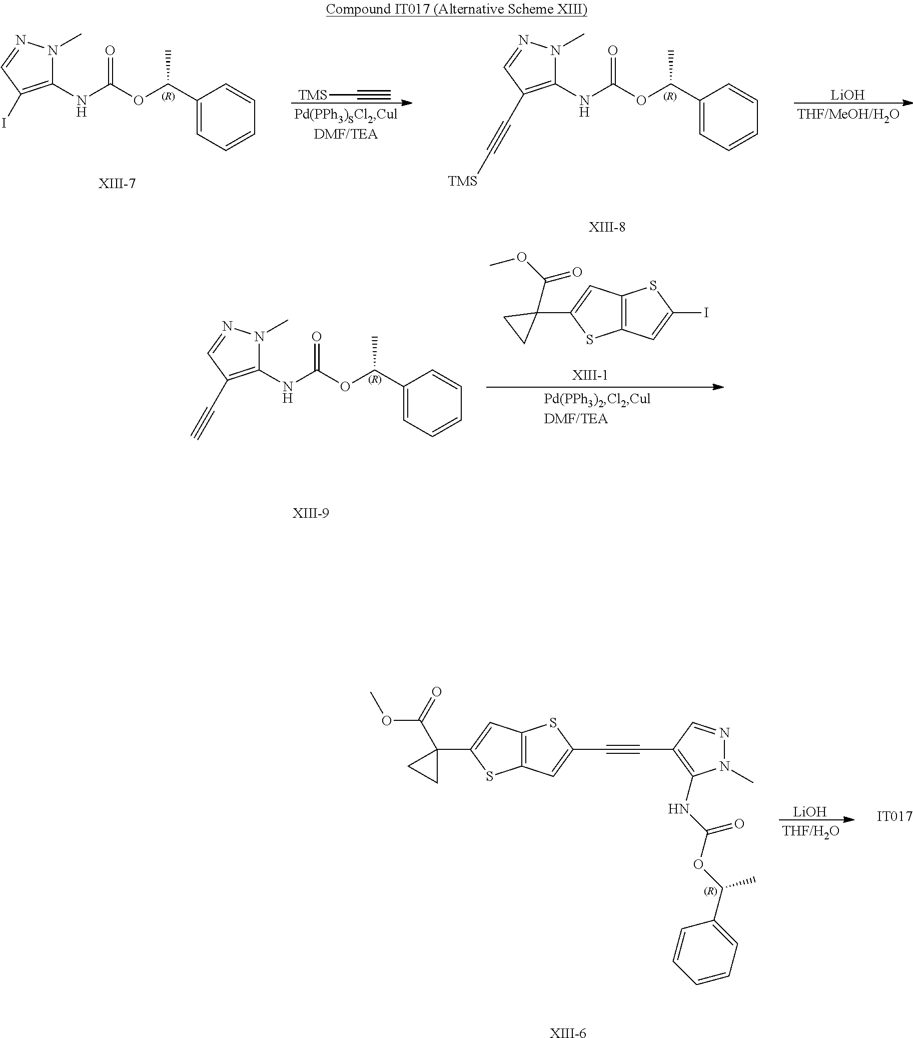

XIII-7 was prepared following the same procedure for the synthesis of XII-5.

The mixture of XIII-7 (500 mg, 1.35 mmol), ethynyltrimethylsilane (264 mg, 2.7 mmol), Pd(PPh₃)₂Cl₂ (94.45 mg, 0.135 mmol) and CuI (25.65 mg, 0.135 mmol) in DMF/Et₃N (20 mL, v/v=3:1) was stirred at rt under nitrogen for 2 h. After concentrated, the residue was partitioned between H₂O and DCM. The aqueous phase was extracted with DCM, and the combined organic layer was washed with brine, dried over Na₂SO₄, concentrated. The residue was purified by chromatography on silica gel (PE:EA=4:1) to afford XIII-8 (400 mg, yield 86.96%).

To a solution of XIII-8 (400 mg, 1.17 mmol) in MeOH (2 mL), THF (2 mL) and H₂O (3 mL), was added LiOH.H₂O (245.6 mg, 5.85 mmol). The reaction mixture was stirred at rt for 2 h. After concentrated, the residue was partitioned between H₂O and EA, the aqueous phase was extracted with EA, and the combined organic layer was washed with brine, dried over Na₂SO₄, concentrated. The residue was purified by chromatography on silica gel (PE:EA=4:1) to afford XIII-9 (220 mg, yield: 70.29%).

To a mixture of XIII-1 (379 mg, 1.04 mmol), CuI (14.06 mg, 0.074 mmol) and Pd (PPh₃)₂Cl₂ (52.11 mg, 0.074 mmol) in DMF/TEA (4 mL, v/v=1/3) was added PhCCH (1.02 mg, 0.01 mmol). The reaction mixture was stirred for 2 min and then XIII-9 (200 mg, 0.74 mmol, in DMF/TEA) was added. The reaction mixture was stirred for 2 h and diluted with water. The mixture was extracted with DCM, and the combined organic layer was washed with brine, dried over Na₂SO₄, concentrated. The residue was purified by chromatography on silica gel (PE:EA=4:1) to afford XIII-6 (330 mg, yield: 88%).

IT017 and its sodium salt IT017a were prepared following the similar procedure described in the preparation of IT001 and IT001a. IT017: MS (ESI) m/z (M+H)⁺492.2. IT017a: ¹H NMR (DMSO-d₆, 400 MHz) δ9.83 (s, 1H), 7.65 (s, 1H), 7.33-7.35 (m, 6H), 6.84 (s, 1H), 5.75-5.80 (q, 1H), 3.60 (s, 2H), 1.51 (d, J=6.4 Hz, 3H), 1.50 (br, 2H), 0.98 (br, 2H). MS (ESI) m/z (M+H)⁺492.2.

Preparation of potassium salt IT017b: To a solution of IT017 (120 mg, 0.244 mmol) in MeOH (10 mL) was added drop wise a solution of aq. KOH (13.65 mg, 0.244 mmol). The mixture was stirred at rt for 30 min. Then the mixture was concentrated and freeze-dried under vacuum. The product was obtained as potassium salt without further purification. MS (ESI) m/z (M+H)⁺492.2. ¹H NMR (DMSO-d₆, 400 MHz): δ 9.80 (s, 1H), 7.66 (s, 1H), 7.27-7.35 (m, 6H), 6.83 (s, 1H), 5.74-5.77 (m, 1H), 3.60 (s, 3H), 1.50 (d, J=6.4 Hz, 3H), 1.41 (br, 2H), 1.06 (br, 2H).

Preparation of calcium salt IT017c: To a solution of IT017 (200 mg, 0.41 mmol) in MeOH (10 mL) and water (2 mL) was added Ca(OH)₂ (15 mg, 0.205 mmol) portion wise. The mixture was heated at 60° C. for 1 h. Then the mixture was concentrated and freeze-dried under vacuum. The product was obtained as calcium salt without further purification. MS (ESI) m/z (M+H)⁺ 492.2. ¹H NMR (DMSO-d₆, 400 MHz): δ 9.80 (s, 1H), 7.66 (s, 1H), 7.27-7.35 (m, 6H), 6.83 (s, 1H), 5.73-5.76 (m, 1H), 3.59 (s, 3H), 1.49-1.51 (m, 5H), 1.06 (br, 2H).

Preparation of trisamine salt IT017d: To a solution of IT017 (200 mg, 0.407 mmol) in MeOH (10 mL) and water (2 mL) was added trisamine (2-Amino-2-hydroxymethyl-propane-1,3-diol) (49.18 mg, 0.407 mmol) portion wise. The mixture was heated at 60° C. for 1 h. Then the mixture was concentrated and freeze-dried under vacuum. The product was obtained as trisamine salt without further purification. MS (ESI) m/z (M+H)⁺492.2. ¹H NMR (DMSO-d₆, 400 MHz): δ 7.60 (s, 1H), 7.30-7.33 (m, 6H), 7.04 (s, 1H), 5.84-5.89 (m, 1H), 3.69 (s, 3H), 3.65 (s, 6H), 1.59-1.61 (m, 5H), 1.19-1.20 (m, 2H).

IT047 was prepared by reacting XIII-1 with the corresponding acetylene (R)-1-(2-chlorophenyl)ethyl (5-ethynyl-3-methylisoxazol-4-yl)carbamate following the similar procedure in the preparation of I-6, followed by the standard LiOH hydrolysis and NaOH basification. IT047: MS (ESI) m/z (M+H)⁺527.2. IT047a: ¹H NMR (400 MHz, DMSO-d₆): δ9.64 (s, 1H), 7.63 (s, 1H), 7.39-7.52 (m, 2H), 7.32-7.37 (m, 2H), 6.89 (s, 1H), 5.99-6.04 (q, 1H), 2.14 (s, 3H), 1.50-1.52 (m, 5H), 1.01 (br, 2H). MS (ESI) m/z (M+H)⁺527.0.

IT048 was prepared by reacting XIII-1 with the corresponding acetylene benzyl (5-ethynyl-3-methylisoxazol-4-yl)carbamate following the similar procedure in the preparation of 1-6, followed by the standard LiOH hydrolysis and NaOH basification. Sodium salt IT048a: ¹H NMR (400 MHz, DMSO-d₆): δ7.75 (s, 1H), 7.33-7.41 (m, 5H), 6.86 (s, 1H), 5.15 (s, 2H), 2.11 (s, 3H), 1.47-1.52 (m, 2H), 0.93-1.04 (m, 2H). MS (ESI) m/z (M+H)⁺479.1.

IT070 was prepared by following the similar alternative synthetic scheme XIII of IT017 using (R)-1-phenylethyl (1-ethyl-4-ethynyl-1H-pyrazol-5-yl)carbamate in place of XIII-9. MS (ESI) m/z (M+H)⁺506.0. Sodium salt IT070a: ¹H NMR (DMSO-d₆, 400 MHz): δ 9.70 (s, 1H), 7.69 (s, 1H), 7.26-7.34 (m, 6H), 6.86 (s, 1H), 5.78 (q, J=6.4 Hz, 1H), 3.94 (q, J=6.8 Hz, 2H), 1.46-1.51 (m, 5H), 1.24 (t, J=7.6 Hz, 3H), 1.00 (br, 2H). MS (ESI) m/z (M+H)⁺506.0.

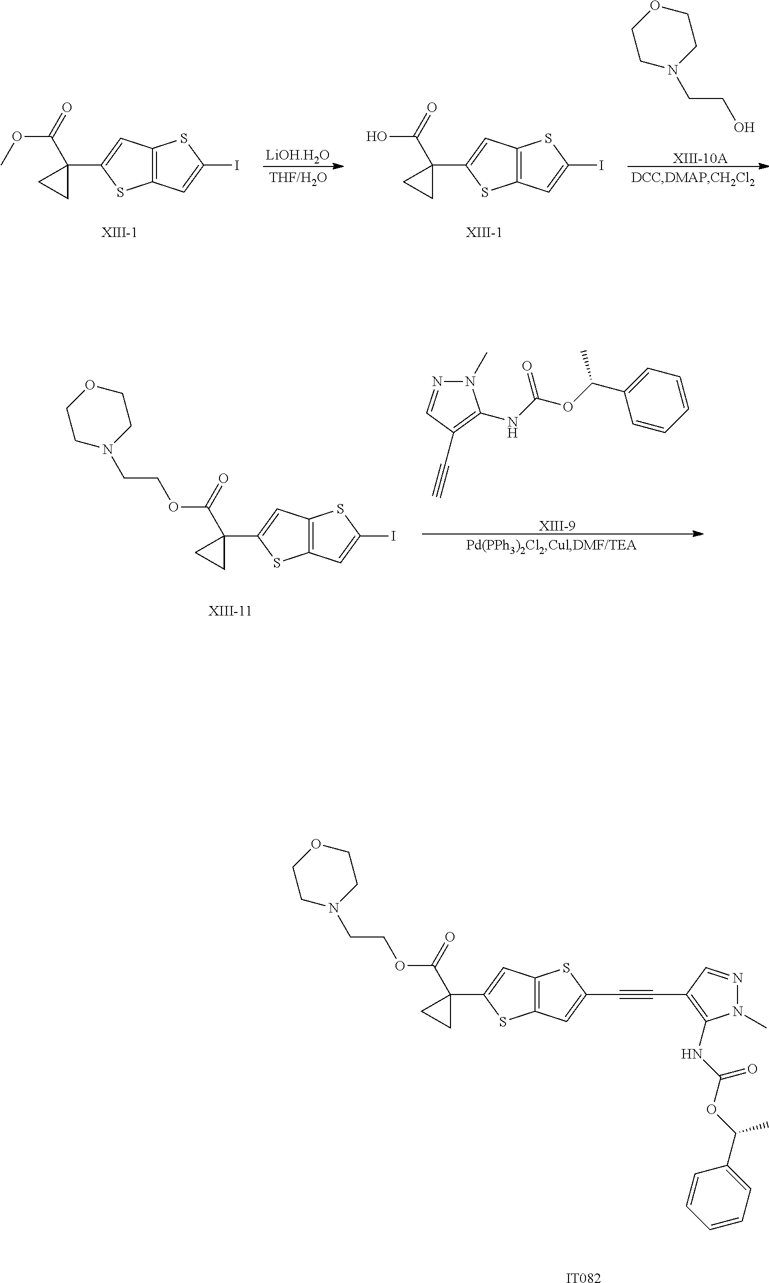

XIII-10 was obtained from XIII-1 by LiOH hydrolysis. To a solution of XIII-10 (100 mg, 0.286 mmol) in CH₂Cl₂ (3 mL) was added DCC (53 mg, 0.257 mmol) and DMAP (3.49 mg, 0.03 mmol). After 30 min, XIII-10A (37.4 mg, 0.286 mmol) was added. Then the mixture was stirred at 25° C. for 3 hrs. After concentrated, the mixture was diluted with EtOAc, washed with brine, dried over Na₂SO₄, filtered and concentrated. The residue was purified by column over silica gel (PE/EA=2/1) to afford XIII-11 (55 mg, yield 41.7%).

XIII-11 (55 mg, 0.12 mmol), Pd(PPh₃)₂Cl₂ (8.4 mg, 0.012 mmol), and CuI (2.3 mg, 0.012 mmol) were mixed with DMF (3 mL) and Et₃N (1 mL) under argon atmosphere. Then a solution of XIII-9 (35 mg, 0.13 mmol) in DMF (1.5 mL) and Et₃N (0.5 mL) was added slowly at rt. The mixture was stirred at rt for 2 hrs. Then the mixture was diluted with EtOAc, washed with brine, dried over Na₂SO₄, filtered and concentrated. The residue was purified by column over silica gel (DCM/MeOH=10/1) to afford IT082 (50 mg, yield 69.4%). ¹H NMR (400 MHz, DMSO-d₄): δ 9.79 (br, 1H), 7.68 (s, 1H), 7.46 (s, 1H), 7.27-7.36 (m, 6H), 5.77 (q, J=6.0 Hz, 1H), 4.14 (t, J=5.2 Hz, 2H), 3.61 (s, 1H), 3.43-3.46 (m, 4H), 2.46 (t, J=5.2 Hz, 2H), 2.28 (br, 4H), 1.63-1.66 (m, 2H), 1.50-1.52 (d, J=6.0 Hz, 3H), 1.44-1.45 (m, 2H). MS (ESI) m/z (M+H)⁺605.0.

IT083 was prepared by first hydrolyzing XIII-1 with NaBH₄ and CaCl₂ in EtOH to afford an intermediate alcohol, followed by Suzuki coupling with XIII-9 as described above in the synthesis of IT082. ¹H NMR (400 MHz, Methanol-d₄): δ 7.58 (s, 1H), 7.20-7.37 (m, 6H), 7.12 (s, 1H), 5.84 (q, J=6.0 Hz, 1H), 3.65-3.69 (m, 5H), 1.57 (d, J=6.0 Hz, 3H), 1.02 (brs, 4H). MS (ESI) m/z (M+H)⁺478.0.

IT084 was prepared by DCC coupling of XIII-10 with 2-methoxyethanol following the similar procedure described in the synthesis of XIII-11, followed by Suzuki Coupling with XIII-9 as described above in the synthesis of IT082. ¹H NMR (DMSO-d₆, 400 MHz): δ 9.81 (br, 1H), 7.70 (s, 1H), 7.48 (s, 1H), 7.29-7.38 (m, 6H), 5.80 (q, J=6.4 Hz, 1H), 4.19 (t, J=4.8 Hz, 2H), 3.63 (s, 1H), 3.50 (t, J=4.8 Hz, 2H), 3.22 (s, 3H), 1.67-1.70 (m, 2H), 1.53 (d, J=6.4 Hz, 3H), 1.47-1.49 (m, 2H). MS (ESI) m/z (M+H)⁺550.0.

IT085 was prepared by reacting XIII-10 with ethyl iodide to form the corresponding ethyl ester, then Suzuki Coupling with XIII-9 as described above in the synthesis of IT082. ¹H NMR (CDCl₃, 400 MHz): δ7.60 (s, 1H), 7.31-7.35 (m, 5H), 7.24 (s, 1H), 7.06 (s, 1H), 6.43 (s, 1H), 5.90 (q, J=6.8 Hz, 1H), 4.18 (q, J=7.2 Hz, 2H), 3.73 (s, 3H), 1.74-1.77 (m, 2H), 1.59-1.62 (m, 5H), 1.39-1.40 (m, 2H), 1.24 (t, J=7.2 Hz, 3H). MS (ESI) m/z (M+H)⁺520.0.

IT086 was prepared by reacting XIII-10 with isopropyl iodide to form the corresponding isopropyl ester, then Suzuki Coupling with XIII-9 as described above in the synthesis of IT082. ¹H NMR (CDCl₃, 400 MHz): δ7.61 (s, 1H), 7.30-7.38 (m, 5H), 7.24 (s, 1H), 7.04 (s, 1H), 6.44 (br, 1H), 5.90 (q, J=6.8 Hz, 1H), 4.99-5.06 (m, 1H), 3.73 (s, 3H), 1.72-1.75 (m, 2H), 1.59-1.62 (m, 3H), 1.37-1.39 (m, 2H), 1.21-1.23 (m, 6H). MS (ESI) m/z (M+H)⁺534.0.

IT087 was prepared by reacting XIII-10 with chloromethyl pivalate in THF in the presence of Cs₂CO₃, then Suzuki Coupling with XIII-9 as described above in the synthesis of IT082. ¹H NMR (DMSO-d₆ 400 MHz): δ9.78 (s, 1H), 7.68 (s, 1H), 7.46 (s, 1H), 7.27-7.36 (m, 6H), 5.79 (q, J=6.8 Hz, 1H), 5.70 (s, 2H), 3.62 (s, 3H), 1.66 (s, 2H), 1.52 (br, 5H), 1.12 (s, 9H). MS (ESI) m/z (M+H)⁺606.0.

IT088 was prepared by first reacting XIII-10 with 2-methoxyphenol in DCM in the presence of DIEA and HATU to form the corresponding aryl ester, then Suzuki Coupling with XIII-9 as described above in the synthesis of IT082. ¹H NMR (400 MHz, Methanol-d₄): δ 7.60 (s, 1H), 7.30-7.34 (m, 2H), 7.23-7.25 (m, 3H), 7.07-7.09 (m, 3H), 7.01-7.02 (m, 2H), 6.92-6.97 (s, 1H), 5.83-5.88 (q, J=6.4 Hz, 1H), 3.85 (s, 3H), 3.69 (s, 3H), 1.95-1.98 (m, 2H), 1.58-1.63 (m, 5H). MS (ESI) m/z (M+H)⁺598.0.

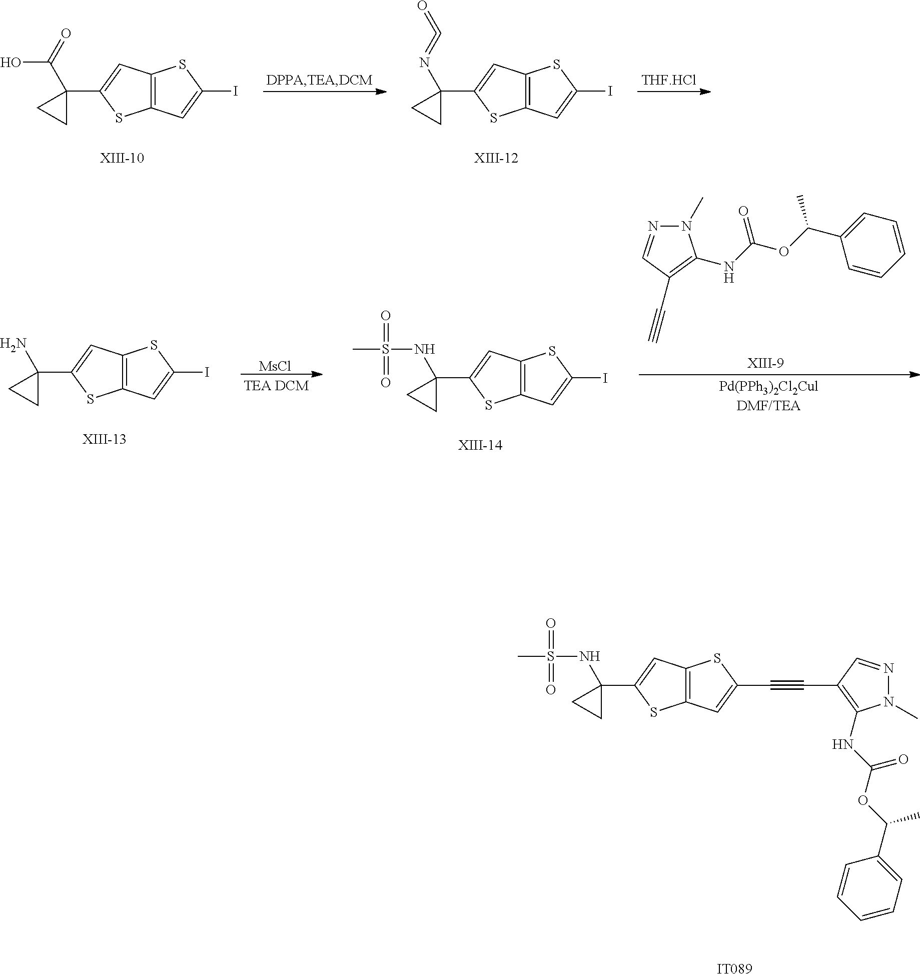

To a solution of XIII-10 (500 mg, 1.43 mmol) in DCM (12 mL) was added DPPA (470 mg, 1.7 mmol) and TEA (286 mg, 2.86 mmol). The reaction mixture was stirred at rt overnight. The mixture was diluted with DCM, washed with brine, and concentrated. The residue was purified by column (PE/EA=10/1) to give XIII-12 (400 mg, yield: 81%).

To a solution of XIII-12 (700 mg, 1.6 mmol) in THF (10 mL) was added 6N HCl (10 mL). The reaction mixture was heated to 70° C. and stirred for 6 h. The mixture was diluted with EtOAc, washed with brine, dried over Na₂SO₄, filtered and concentrated to give XIII-13 (600 mg, yield: 92%).

To a stirred mixture of XIII-13 (600 mg, 1.87 mmol), TEA (374 mg, 3.74 mmol) in DCM (10 mL) was added MsCl (234 mg, 2.06 mmol). The reaction mixture was flushed with nitrogen and stirred for 1 h at 25° C. The mixture was concentrated and diluted with EtOAc, washed with water and brine, dried over Na₂SO₄, filtered and concentrated. The mixture was concentrated and purified by column (PE/EA=5/1) to give XIII-14 (600 mg, yield: 80%).

IT089 was prepared by Suzuki Coupling of XIII-14 with XIII-9 as described above in the synthesis of IT082. ¹H NMR (400 MHz, DMSO-d₆): δ9.79 (s, 1H), 8.43 (s, 1H), 7.70 (s, 1H), 7.48 (s, 1H), 7.33-7.38 (m, 4H), 7.26-7.30 (m, 2H), 5.77-5.82 (q, 1H), 3.63 (s, 3H), 2.74 (s, 3H), 1.52-1.54 (d, J=6.4 Hz, 3H), 1.42-1.43 (m, 2H), 1.26-1.29 (m, 2H). MS (ESI) m/z (M+H)⁺ 541.0.

IT090 was prepared by reacting XIII-10 with 2-chloro-N,N-dimethylacetamide in DMF in the presence of Cs₂CO₃, then Suzuki Coupling with XIII-9 as described above in the synthesis of IT082. ¹H NMR (CDCl₃, 400 MHz) δ 7.59 (s, 1H), 7.28-7.36 (m, 5H), 7.22 (s, 1H), 7.16 (s, 1H), 6.52 (br, 1H), 5.97 (q, J=6.4 Hz, 1H), 4.43 (s, 2H), 3.74 (s, 3H), 2.95 (s, 3H), 2.93 (s, 3H), 1.89-1.92 (m, 2H), 1.62 (s, 3H), 1.45-1.48 (m, 2H). MS (ESI) m/z (M+H)⁺577.0.

IT097 was prepared following the alternative synthesis of IT017 using (R)-1-phenylethyl (4-iodo-1-methyl-1H-pyrazol-5-yl)(methyl)carbamate in place of XIII-7. MS (ESI) m/z (M+H)⁺505.9. Sodium salt IT097a: ¹H NMR (DMSO-d₆, 400 MHz): δ 7.66 (s, 1H), 7.36 (s, 1H), 7.26-7.31 (m, 5H), 6.83 (s, 1H), 5.78-5.83 (q, J=6.4 Hz, 1H), 3.64 (s, 3H), 3.25 (s, 3H), 1.49 (d, J=6.4 Hz, 3H), 1.46-1.47 (m, 2H), 0.90-0.93 (m, 2H). MS (ESI) m/z (M+H)⁺506.0.

IT098 was prepared by reacting XIII-10 with methanesulfonamide in the presence of HATU and DIEA in DCM, followed by Suzuki coupling with XIII-9 using the same procedure described above. ¹H NMR (400 MHz, DMSO-d₆): δ 9.80 (brs, 1H), 7.60 (s, 1H), 7.28-7.41 (m, 7H), 5.77-5.78 (q, 1H), 3.61 (s, 3H), 2.99 (s, 3H), 1.51-1.52 (m, 5H), 1.23 (brs, 2H). MS (ESI) m/z (M+H)⁺568.9.

IT099 was prepared by two-step reduction reactions of IT017. First, a mixture of IT017 (0.2 g, 0.406 mol) and PtO₂ (20 mg) in MeOH (10 mL) was hydrogenated under 45 Psi of hydrogen pressure for 2 h at rt. The suspension was filtered through a pad of silica gel and the filter cake was washed with MeOH. The combined filter was concentrated to give an intermediate (160 mg, yield: 79.68%), which was mixed with and Pd/C (20 mg) in MeOH (10 mL) and hydrogenated under 45 Psi of hydrogen pressure for 2 h at rt. The suspension was filtered through a pad of silica gel and the filter cake was washed with MeOH. The organic layers was washed with brine, dried over Na₂SO₄, and concentrated. The residue was purified by prep-HPLC to afford IT099 (100 mg, yield: 62.66%). MS (ESI) m/z (M+H)⁺ 495.9. Sodium salt IT099a: ¹H NMR (DMSO-d₆, 400 MHz): δ9.54 (s, 1H), 7.28-7.29 (m, 5H), 7.21 (s, 1H), 6.86 (s, 1H), 6.83 (s, 1H), 5.75 (d, J=6.4 Hz, 1H), 3.53 (s, 3H), 2.93 (t, J=7.6 Hz, 2H), 2.25 (t, J=7.6 Hz, 2H), 1.51 (br, 2H), 1.39 (br, 3H), 0.95 (br, 2H). MS (ESI) m/z (M+H)⁺495.9.

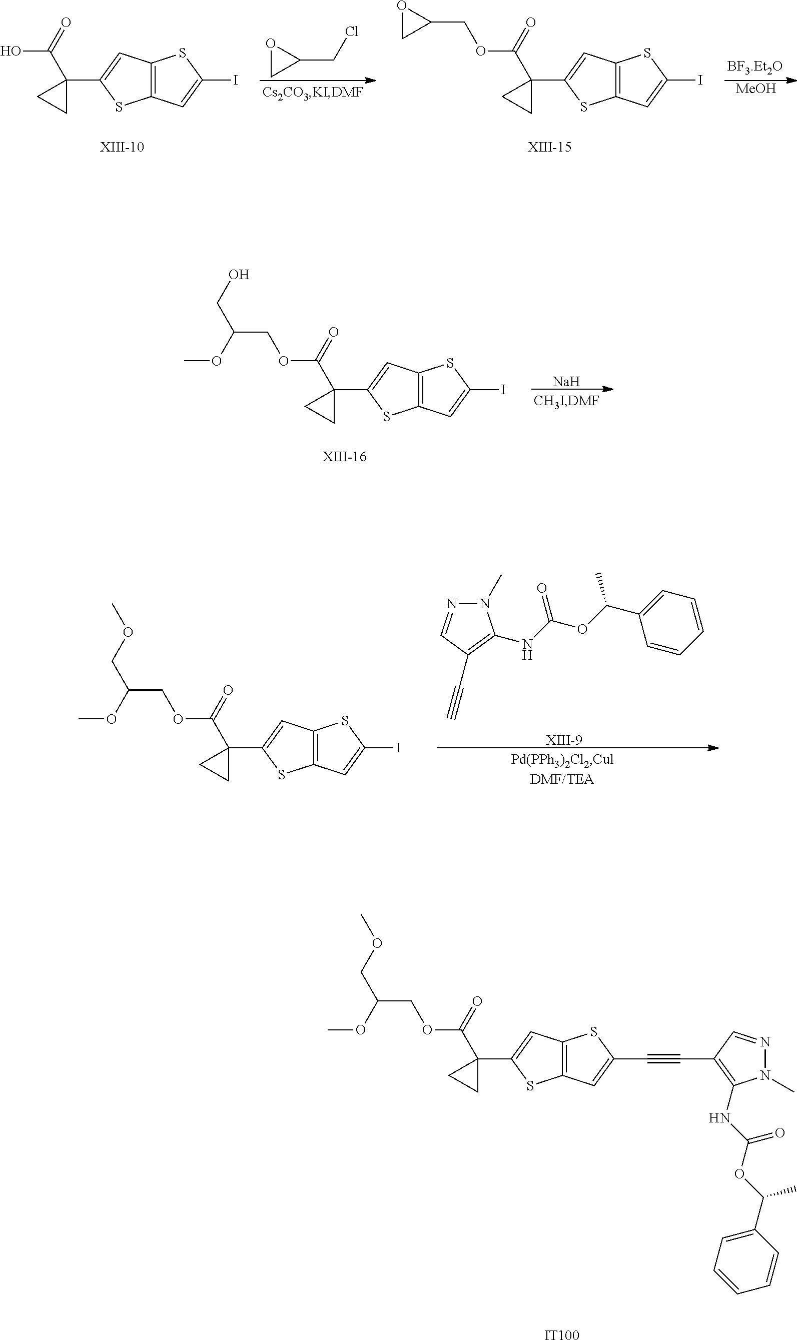

To a solution of XIII-10 (500 mg, 1.43 mmol) in DMF (10 mL) was added Cs₂CO₃ (930 mg, 2.86 mmol), KI (23 mg, 0.143 mmol), and 2-(chloromethyl)oxirane (160 mg, 1.74 mmol). The reaction mixture was heated at 70° C. for 12 h. The mixture was washed with water, extracted with EtOAc. The organics were combined, washed with saturated NaHCO₃, brine, dried with Na₂SO₄, filtered and concentrated to afford XIII-15 (165 mg, yield: 28.4%).

To a solution of XIII-15 (83 mg, 0.2 mmol) in MeOH (10 mL) was added BF₃.Et₂O (15 mg, 0.1 mmol) at −34° C. Then the reaction mixture was stirred at 4° C. for 12 h. The mixture was diluted with EtOAc, washed with H₂O. The organics were combined, washed brine, dried with Na₂SO₄, filtered and concentrated. The residue was purified by prep-TLC (PE/EA=3/1) to give XIII-16 (50 mg, yield: 55.8%).

To a solution of XIII-16 (25 mg, 0.057 mmol) and CH₃I (12 mg, 0.085 mmol) in DMF (2.5 mL) was added NaH (3 mg, 0.075 mmol, 60%) at −20° C. The mixture was stirred at 4° C. for 12 h. Then the mixture was quenched with H₂O, and extracted with EtOAc. The organics were combined, washed brine, dried with Na₂SO₄, filtered and concentrated. The residue was purified by prep-TLC (PE/EA=3/1) to give XIII-17 (8 mg, yield: 31%).

IT100 was obtained by Suzuki Coupling of XIII-17 and XIII-9 using the procedure described above. ¹HNMR (Methanol-d₄, 400 MHz) δ 7.61 (s, 1H), 7.22-7.41 (m, 7H), 5.84-5.89 (q, 1H), 4.28-4.30 (m, 1H), 4.07-4.12 (m, 1H), 3.70 (s, 3H), 3.51-3.53 (m, 1H), 3.39-3.40 (m, 2H), 3.38 (s, 3H), 3.30 (s, 3H), 1.73-1.76 (m, 2H), 1.60 (d, J=6.4 Hz, 3H), 1.47-1.49 (m, 2H). MS (ESI) m/z (M+H)⁺594.0.

IT101 was prepared following the similar procedure described in the alternative synthesis of IT017 using (R)-1-phenylethyl (5-ethynylthiazol-4-yl)carbamate in place of XIII-9. MS (ESI) m/z (M+H)⁺495.1. ¹H NMR (Methanol-d₄, 400 MHz): δ 8.83 (s, 1H), 7.40-7.42 (d, J=7.2 Hz, 2H), 7.30-7.34 (m, 2H), 7.25-7.27 (m, 2H), 7.19 (s, 1H), 5.87 (q, J=6.4 Hz, 1H), 1.74-1.76 (m, 2H), 1.59 (d, J=6.4 Hz, 3H), 1.44-1.46 (m, 2H).

Preparation of IT103: t-BuOOH (185 mg, 2.04 mmol) was added to a solution of IT017 (200 mg, 0.407 mmol), NaSO₂CF₃ (190 mg, 1.22 mmol) and CuSO₄ (6.4 mg, 0.04 mmol) in DMSO (10 mL). The reaction mixture was stirred at 30° C. for 24 h. Then additional t-BuOOH (185 mg, 2.04 mmol) and NaSO₂CF₃ (190 mg, 1.22 mmol) was added to the reaction mixture. The reaction mixture was stirred at 30° C. for additional 24 h. The reaction mixture was diluted with EtOAc and water. The aqueous layer was separated and extracted with EtOAc. Following standard work-up procedure, the filtrate was evaporated in vacuum and the residue was purified by prep-HPLC (containing 0.1% HCl) to afford IT103 (21 mg, yield 9.2%,). ¹H NMR (DMSO-d₆, 400 MHz): δ 12.9 (br, 1H), 9.84 (br, 1H), 7.75 (s, 1H), 7.32-7.40 (m, 5H), 7.27-7.29 (m, 1H), 5.79 (t, J=6.0 Hz, 1H), 3.65 (s, 3H), 1.68-1.71 (m, 2H), 1.53 (d, J=6.0 Hz, 3H), 1.45-1.47 (m, 2H). MS (ESI) m/z (M+H)⁺559.9.

### Example 6-C

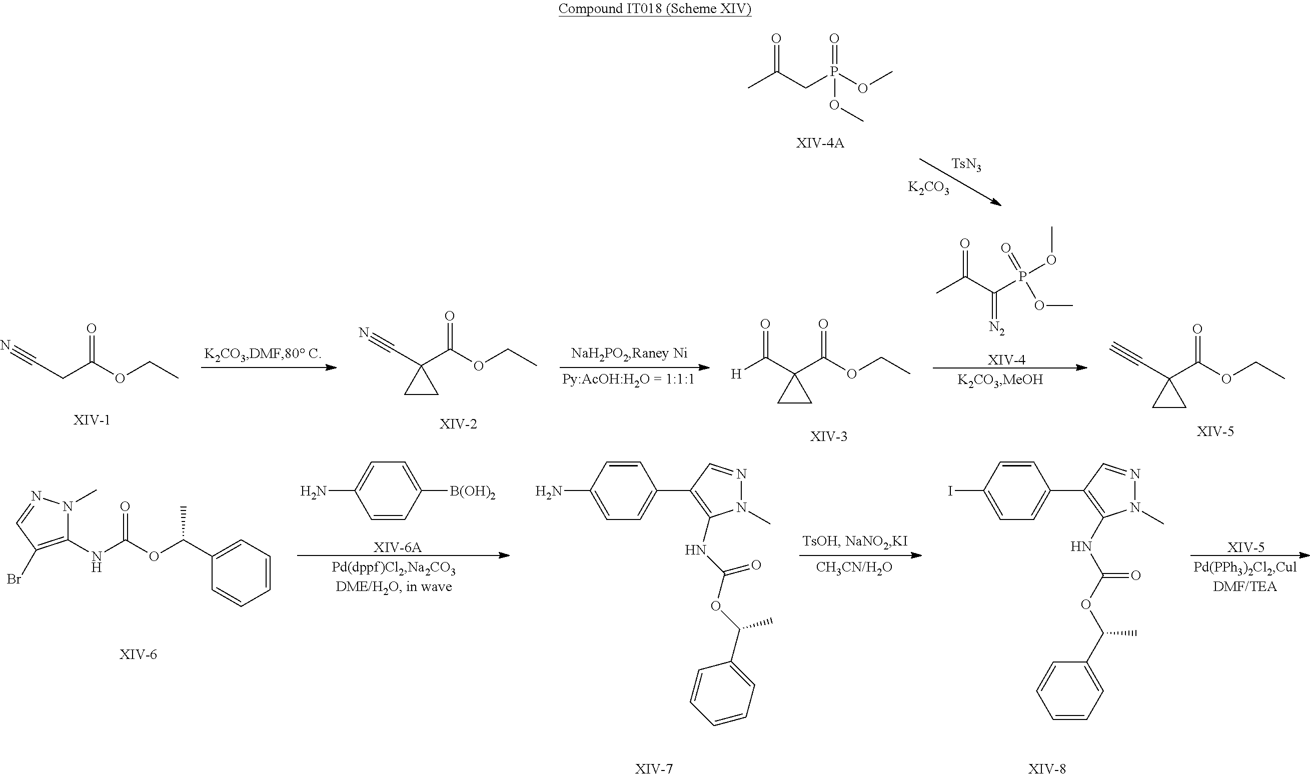

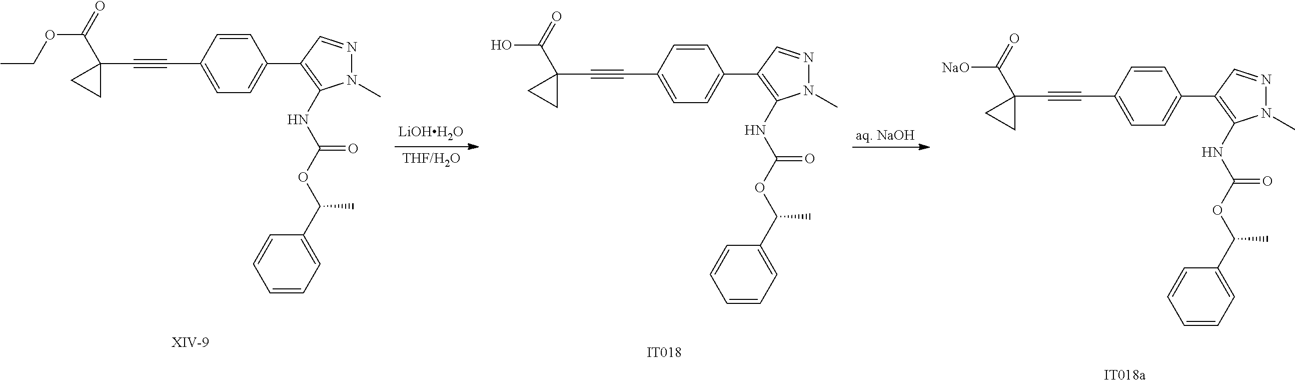

XIV-5 was prepared from ethyl 2-cyanoacetate in three steps reactions.

To a solution of XIV-6 (500 mg, 1.55 mmol) in DME/H₂O (v/v=3/1, 8 mL), Na₂CO₃ (821 mg, 7.75 mmol) and XIV-6A (592 mg, 2.32 mmol) were added, the resulting mixture was purged with nitrogen, then Pd (dppf)Cl₂ (113 mg, 0.16 mmol) was added. The reaction mixture was heated to 110° C. for 60 min. under nitrogen protection. After completion of the reaction, the mixture was poured into water, extract with EtOAc (10 mL×3). The combined organic layers were dried over Na₂SO₄, concentrated in vacuo. The residue was purified by chromatography (PE:EA=1:1) to afford XIV-7 (300 mg, yield: 57.7%).

To a solution of p-TsOH (700 mg, 3.57 mmol) in CH₃CN (1 mL) was added dropwise XIV-7 (400 mg, 1.19 mmol) in CH₃CN (2 mL), then the stirred mixture was cooled to 10-15° C. KI (492 mg, 2.98 mmol) and NaNO₂ (164 mg, 2.38 mmol) in H₂O (1.5 mL) was added to the reaction mixture. After addition, the mixture was stirred at rt for 3 hrs. After completion of the reaction, the mixture was poured into water, extract with EtOAc. The combined organic layers were washed with aq. Na₂SO₃, brine and dried over Na₂SO₄, concentrated in vacuo. The residue was purified by chromatography (PE:EA=2:1) to afford XIV-8 (350 mg, yield: 66%).

To a stirred mixture of XIV-8 (344 mg, 0.77 mmol), XVI-5 (crude) and CuI (49 mg, 0.26 mmol) in DMF (5 mL) and TEA (1 mL) was added Pd(PPh₃)₂Cl₂ (54 mg, 0.08 mmol). The reaction mixture was flushed with N₂ and stirred at rt overnight. The mixture was diluted with EA, washed with water and brine, dried over Na₂SO₄, filtered and concentrated. The residue was purified by chromatography (PE:EA=2:1) to afford XIV-9 (380 mg, crude yield: 100%).

IT018 and its sodium salt IT018a were prepared following the similar procedure described in the preparation of IT001 and IT001a. IT018: MS (ESI) m/z (M+H)⁺430.1. IT018a: ¹HNMR (Methanol-d₄, 400 MHz): δ7.70 (s, 1H), 7.19-7.40 (m, 9H), 5.81 (br, 1H), 3.68 (s, 3H), 1.59 (br, 3H), 1.45 (d, J=3.6 Hz, 2H), 1.14 (d, J=3.2 Hz, 2H). MS (ESI) m/z (M+H)⁺430.1.

### Example 7

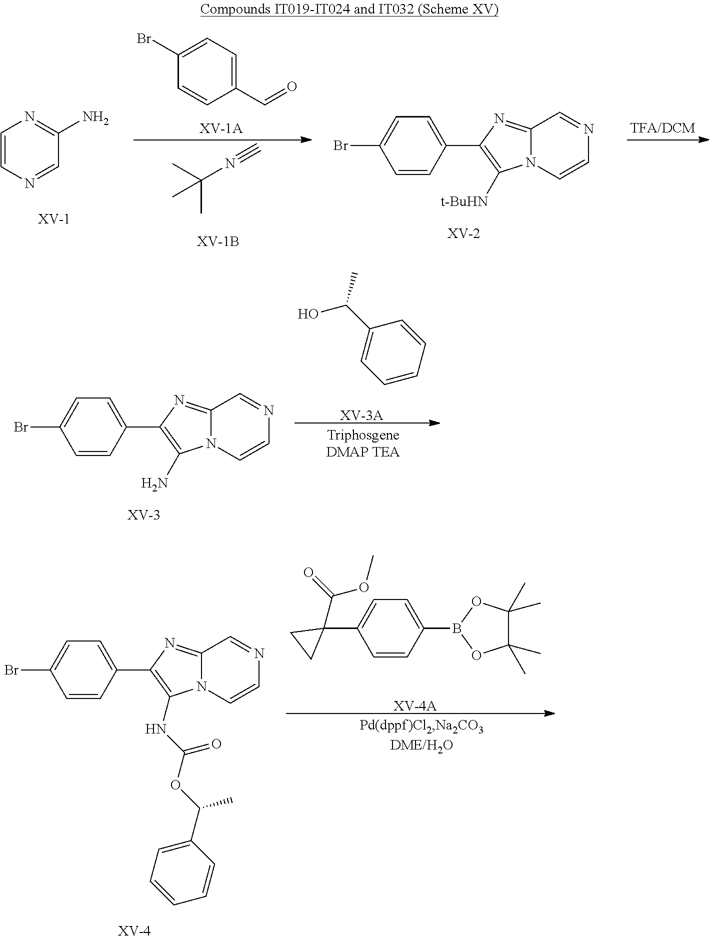

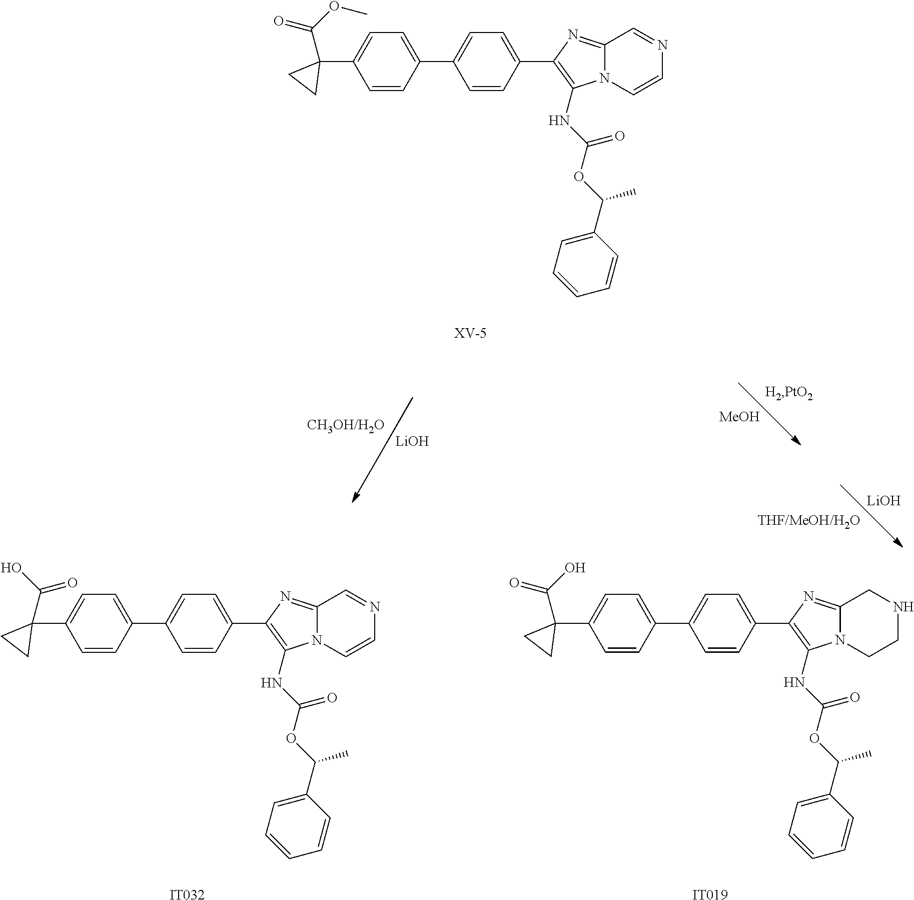

To XV-1 (1.08 g, 11.39 mmol) in 20 mL of MeOH were added XV-1A (3.39 g, 17 mmol), XV-1B (1.1 g, 13.1 mmol) and 1.0 N HClO₄ in MeOH (1.14 mL, 1.14 mmol). The reaction mixture was stirred at rt for 8 h. Solvent was removed and the residue was purified by flash chromatography (PE:EA=1:1) to give XV-2 (2.5 g, yield: 64.1%).

A solution of XV-2 (2.5 g, 7.27 mmol) in 30 mL of DCM/TFA (v/v=4/1) was stirred at rt for 2 h. Solvent was removed, after neutralization with aqueous NaHCO₃, XV-3 (3.5 g, crude) was obtained and directly used in the next step.

To a solution of XV-3 (400 mg, 1.384 mmol) in 1,2-dichloroethane (10 mL) was added (R)-1-phenylethanol (422 mg, 6.92 mmol), TEA (699 mg, 6.92 mmol) and DMAP (168 mg, 0.692 mmol). The reaction mixture was stirred at rt for 6 h. The mixture was diluted with EtOAc, washed with brine, dried over Na₂SO₄, filtered and concentrated. The residue was purified by column chromatography (PE:EA=10:1) to give XV-4 (250 mg, yield: 41%).

XV-5 was prepared following the similar procedure as describe in the synthesis of III-5. MS (ESI) m/z (M+H)⁺532.2.

IT032 and its sodium salt IT032a were prepared following the similar procedure described in the synthesis of IT001 and IT001a. IT032: MS (ESI) m/z (M+H)⁺518.2. IT032a: ¹H NMR (DMSO-d₆ 400 MHz) δ9.09 (s, 1H), 8.20 (s, 1H), 8.02 (d, J=7.6 Hz, 2H), 7.95 (d, J=4.4 Hz, 1H), 7.74 (d, J=6.4 Hz, 2H), 7.64 (d, J=8.0 Hz, 3H), 7.36-7.48 (m, 7H), 5.81 (q, 1H), 1.61 (br, 3H), 1.44 (br, 2H), 1.09 (br, 2H). MS (ESI) m/z (M+H)⁺518.2.

To a solution of XV-5 (200 mg, 0.376 mmol) in 4 mL of MeOH was added PtO₂ (20 mg). The reaction mixture was evacuated and back-filled with H₂ for 2 h at 40° C. LCMS showed that that the reaction was completed. The suspension was filtered through a pad of Celite and washed with MeOH (10 mL). The combined filtrates were concentrated and dissolved in MeOH:THF:H₂O=1:1:1 (12 mL). After hydrolysis with LiOH (78 mg, 1.86 mmol) overnight at rt, the solution was concentrated in vacuo, acidified, and extracted with EtOAc. The organic layer was isolated, concentrated, and purified to afford IT019 (120 mg, yield: 61.6%). MS (ESI) m/z (M+H)⁺ 523.2. Sodium salt IT019a: ¹H NMR (DMSO-d₆, 400 MHz): δ7.74 (d, J=8.0 Hz, 2H), 7.28-7.49 (m, 11H), 5.73-5.81 (q, 1H), 3.80 (d, J=5.2 Hz, 2H), 3.55 (br, 2H), 2.98 (br, 2H), 2.66 (br, 1H), 1.48 (br, 3H), 1.17 (br, 2H), 0.67 (br, 2H). MS (ESI) m/z (M+H)⁺523.2.

IT020 was prepared following the similar procedure for the preparation of IT019 by reacting XV-5 with acetyl chloride in DCM and TEA, followed by LiOH hydrolysis. MS (ESI) m/z (M+H)⁺565.2. Sodium salt IT020a: ¹H NMR (DMSO-d₆, 400 MHz): δ:9.47 (s, 1H), 7.71 (s, 2H), 7.55 (m, 2H), 7.43-7.48 (m, 6H), 7.29-7.32 (m, 2H), 5.77 (q, 1H), 4.74 (br, 1H), 4.64 (br, 1H), 3.78-3.87 (m, 2H), 3.28 (s, 2H), 2.11 (s, 3H), 1.54-1.55 (m, 2H), 1.20 (br, 2H), 0.72 (br, 2H). MS (ESI) m/z (M+H)⁺565.2.

IT021 was prepared following the similar procedure for the preparation of IT019 by reacting XV-5 with MsCl in DCM and TEA, followed by LiOH hydrolysis. MS (ESI) m/z (M+H)⁺601.2. Sodium salt IT021a: ¹H NMR (DMSO-d₆, 400 MHz): δ 9.61 (s, 1H), 7.36-7.75 (m, 13H), 5.79 (br, 1H), 4.46 (br, 2H), 3.82 (br, 4H), 3.07 (s, 3H), 1.58 (br, 3H), 1.35 (br, 2H), 0.96 (br, 2H). MS (ESI) m/z (M+H)⁺601.2.

IT022 was prepared following the similar procedure for the preparation of IT019 by reacting XV-5 with methylcarbamic chloride in DCM and TEA, followed by LiOH hydrolysis. MS (ESI) m/z (M+H)⁺594.2. Sodium salt IT022a: ¹H NMR (DMSO-d₆, 400 MHz): δ 9.45 (s, 1H), 7.73-7.75 (m, 2H), 7.37-7.59 (m, 11H), 6.82 (d, J=4.0 Hz, 1H), 5.78-5.79 (m, 1H), 4.55 (s, 2H), 4.74 (s, 2H), 3.67-3.77 (m, 4H), 2.61-2.65 (d, J=4.0 Hz, 3H), 1.56-1.57 (d, J=6.4 Hz, 3H), 1.38 (br, 2H), 1.01 (br, 2H). MS (ESI) m/z (M+H)⁺594.2.

IT023 was prepared following the similar procedure for the preparation of IT019 by reacting XV-5 with ethyl carbonochloridate in DCM and TEA, followed by LiOH hydrolysis. MS (ESI) m/z (M+H)⁺595.2. Sodium salt IT023a: ¹H NMR (DMSO-d₆, 400 MHz): δ9.53 (s, 1H), 7.73 (d, J=6.8 Hz, 2H), 7.33-7.58 (m, 11H), 5.78-5.79 (m, 1H), 4.62 (br, 2H), 4.10-4.15 (q, J=7.2 Hz, 1H), 3.82 (br, 2H), 3.72 (br, 2H), 1.56-1.57 (d, J=5.2 Hz, 3H), 1.21-1.25 (m, 5H), 0.77 (br, 2H). MS (ESI) m/z (M+H)⁺595.2.

IT024 was prepared following the similar procedure for the preparation of IT019 by reacting XV-5 with ethyl iodide in DMF and TEA, followed by LiOH hydrolysis. MS (ESI) m/z (M+H)⁺551.2. Sodium salt IT024a: ¹H NMR (DMSO-d₆, 400 MHz): δ7.59-7.66 (m, 8H), 7.39-7.47 (m, 5H), 5.84-5.85 (m, 1H), 4.21 (s, 2H), 4.05 (br, 2H), 3.40 (br, 2H), 3.06 (br, 2H), 1.61-1.62 (m, 5H), 1.32 (d, J=6.8 Hz, 3H), 1.23-1.25 (m, 2H). MS (ESI) m/z (M+H)⁺ 551.2.

### Example 8-A

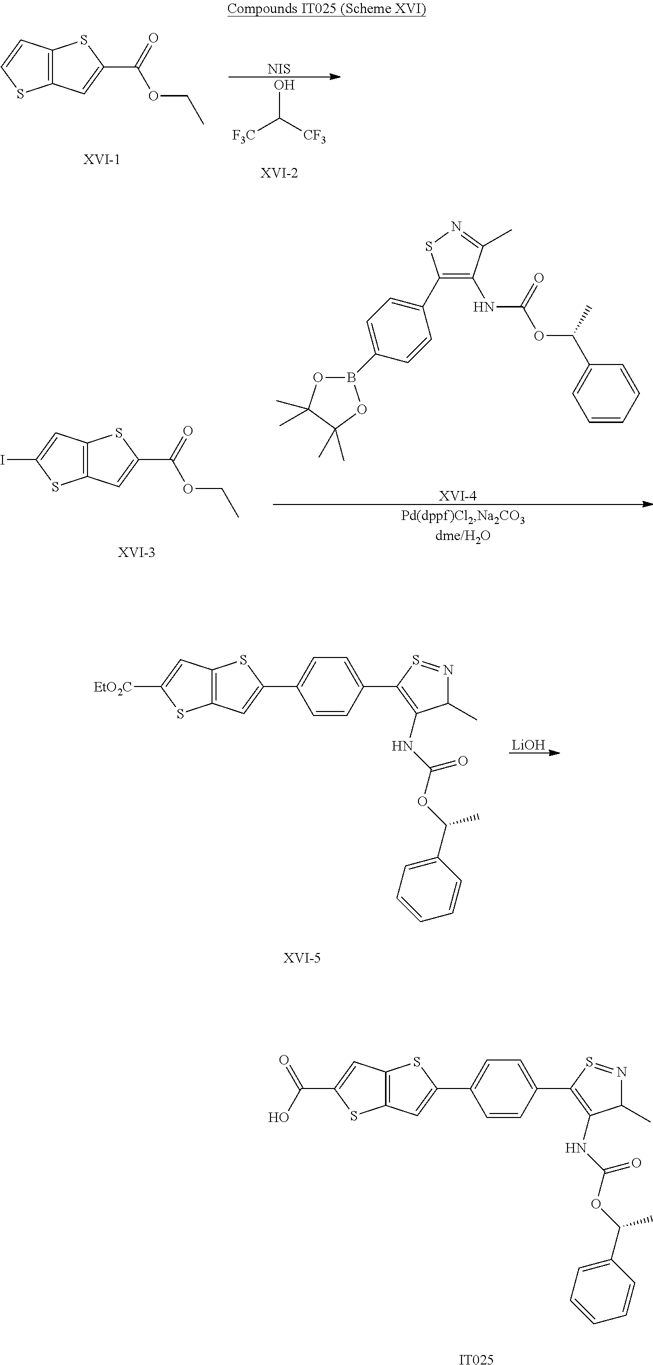

XVI-1 was prepared by reacting 3-bromothiophene-2-carbaldehyde with ethyl 2-mercaptoacetate and K₂CO₃ in DMF at 60° C. overnight under N₂ protection.

To a solution of XVI-1 (2.12 g, 10 mmol) in XVI-2 (10 mL) was added NIS (2.36 g, 10.5 mmol) at rt. The reaction mixture was stirred for overnight, and then the mixture was diluted with water and extracted with EtOAc. The combined organic layers were washed, concentrated under reduced pressure, and purified by column chromatography on silica gel (PE:EA=10:1) to give XVI-3 (2.14 g, yield: 63.3%). ¹H NMR (CDCl₃, 400 MHz): δ7.88 (s, 1H), 7.45 (s, 1H), 4.38 (q, J=7.2 Hz, 2H), 1.40 (t, J=7.2 Hz, 3H).

XVI-5 was prepared by reacting XVI-3 and XVI-4 following the similar procedure as describe in the synthesis of III-5. MS (ESI) m/z (M+H)⁺532.2.

IT025 and its sodium salt IT025a were prepared following the similar procedure described in the synthesis of IT001 and IT001a. IT025a: ¹HNMR (DMSO-d₆, 400 MHz): δ 9.31 (s, 1H), 7.94 (s, 1H), 7.68-7.74 (m, 3H), 7.53-7.54 (m, 2H), 7.33-7.39 (m, 5H), 5.73 (br, 1H), 2.19 (s, 3H), 1.53 (br, 3H). MS (ESI) m/z (M+H)⁺521.0.

### Example 8-B

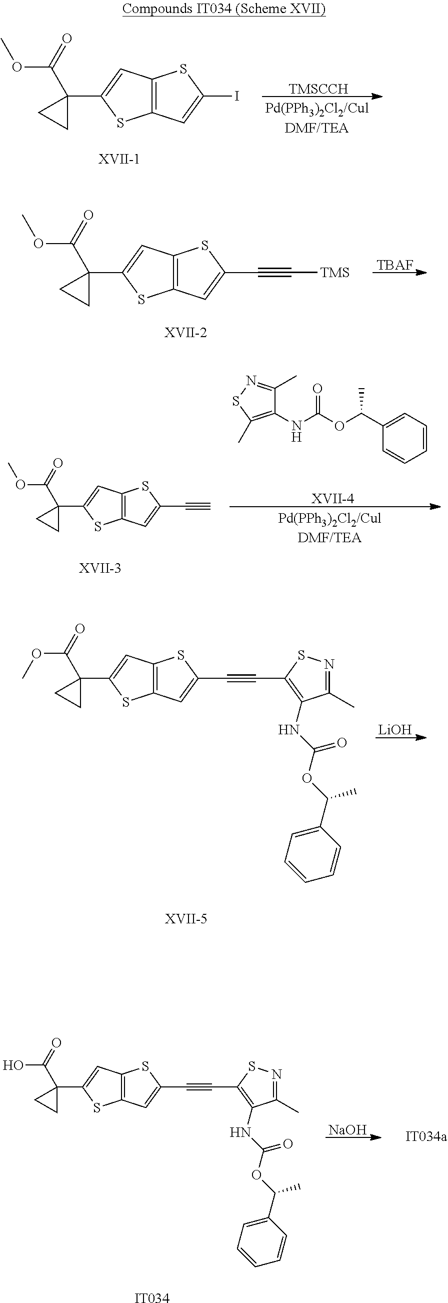

XVII-1 was prepared from XVI-1 by hydrolyzing the ethyl ester into hydroxy with LiAlH₄, converting the hydroxy group into nitrile, cyclization with 1-bromo-2-chloroethane, converting nitrile into methyl ester, and adding the iodo substituent with NIS in five steps.

To a mixture of XVII-1 (1.0 g, 2.75 mmol), CuI (27.7 mg, 0.14 mmol) and Pd(dppf)Cl₂ (96 mg, 0.14 mmol) in DMF/TEA (25 mL, v/v=3:1) was added TMSCCH (0.81 g, 8.24 mmol). The reaction mixture was stirred for 2 h and diluted with water, extracted with EtOAc, and the combined organic layer was washed with brine, dried over Na₂SO₄, concentrated to afford crude XVII-2 (810 mg, crude yield: 88.8%).

To a mixture of compound XVII-2 (810 mg, 242 mmol) in DCM (30 mL) was added TBAF (1.45 g, 6.05 mmol). The reaction mixture was stirred for 2 h and diluted with water, extracted with DCM, and the combined organic layer was washed with brine, dried over Na₂SO₄, concentrated. The residue was purified by chromatography on silica gel (PE:EA=10:1) to afford XVII-3 (390 mg, yield: 61.0%).

XVII-5 was prepared by reacting XVII-3 and XVII-4 following the similar procedure described in the preparation of I-6. MS (ESI) m/z (M+H)⁺523.1.

IT034 and its sodium salt IT034a were prepared following the similar procedure described in the synthesis of IT001 and IT001a. IT034: MS (ESI) m/z (M+H)⁺509.0. IT034a: ¹H NMR (DMSO-d₆, 400 MHz): δ7.49 (s, 1H), 7.30-7.40 (m, 5H), 6.87 (s, 1H), 5.79-5.81 (q, 1H), 2.28 (s, 3H), 1.55 (d, J=6.4 Hz, 3H), 1.48-1.49 (m, 2H), 0.99-1.00 (m, 2H). MS (ESI) m/z (M+H)⁺509.0.

IT074 was prepared following the general synthetic scheme of IT034 replacing XVII-4 with the corresponding carbamate (R)-1-phenylethyl (2-iodobenzofuran-3-yl)carbamate. MS (ESI) m/z (M+H)⁺528.0. Sodium salt IT074a: MS (ESI) m/z (M+H)⁺528.0. ¹H NMR (DMSO-d₆, 400 MHz): δ 9.83 (br s, 1H), 7.52-7.58 (m, 3H), 7.27-7.42 (m, 7H), 6.93 (s, 1H), 5.79-5.84 (q, 1H), 1.55 (d, J=6.4 Hz, 3H), 1.48 (br, 2H), 1.05 (br, 2H).

### Example 9

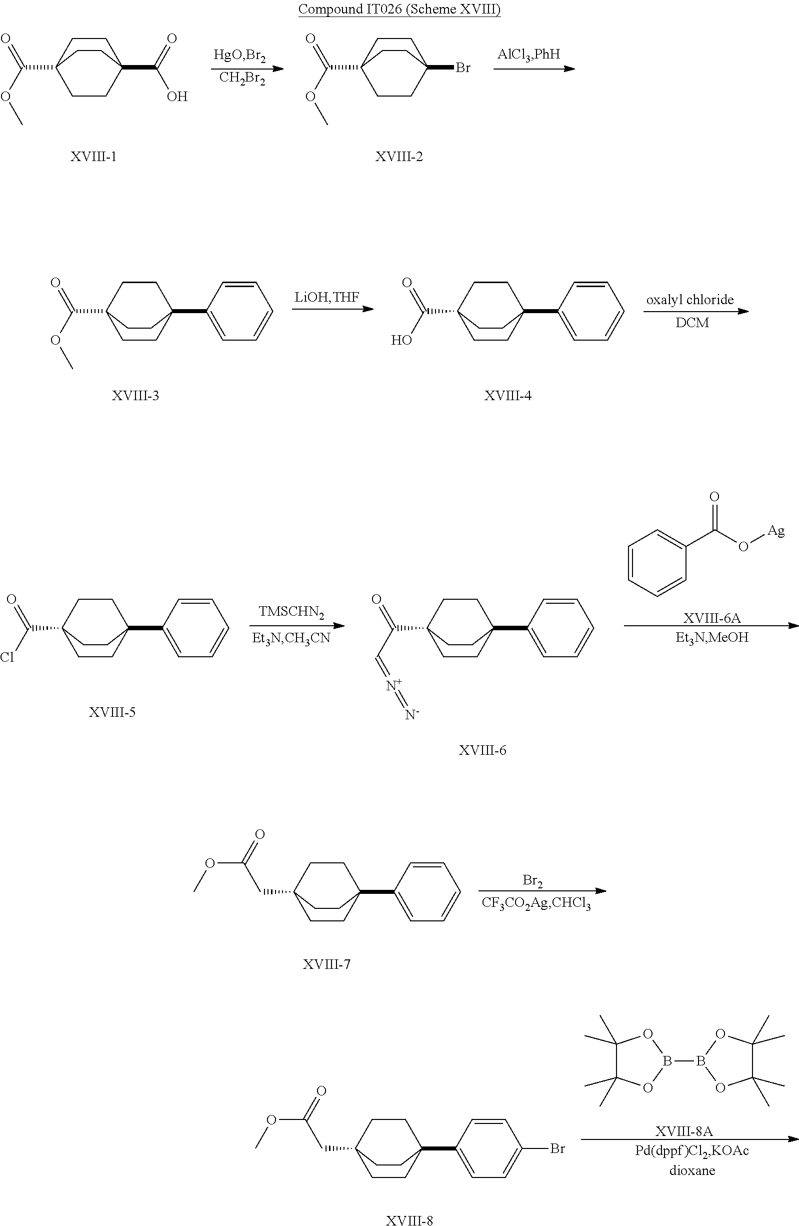

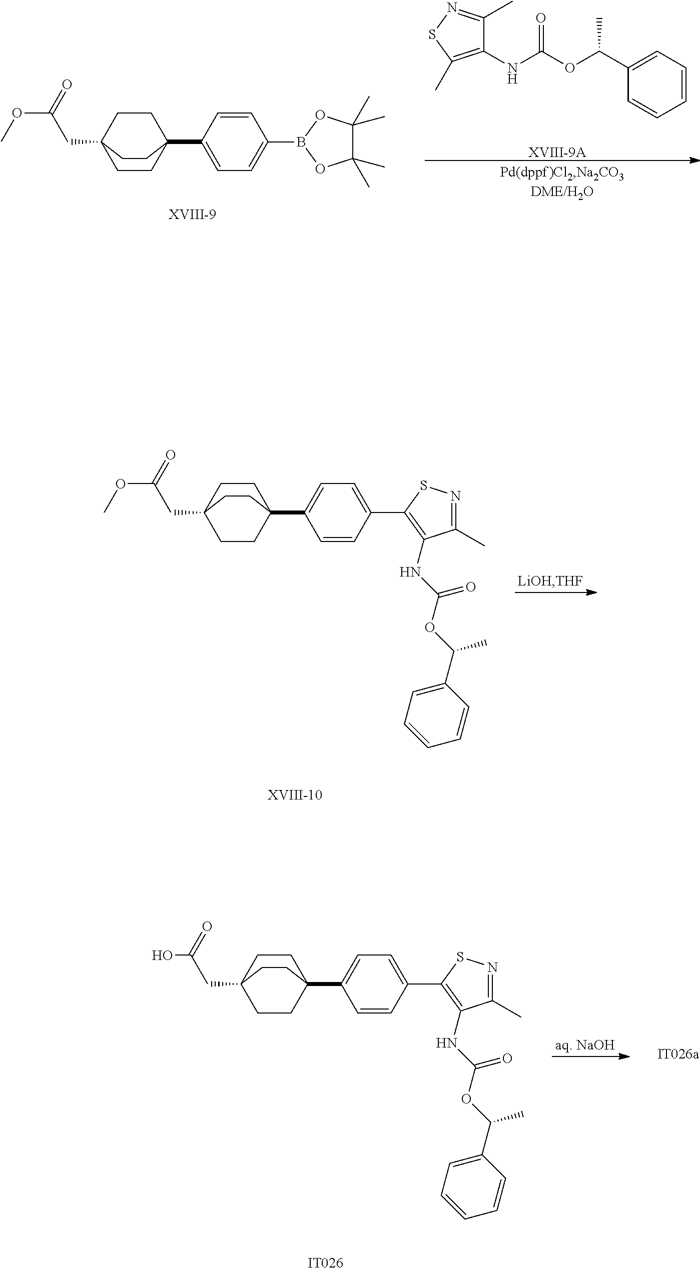

To a stirred mixture of XVIII-1 (10 g, 47.2 mmol) in CH₂Br₂ (100 mL) was added HgO (17.5 g, 80.3 mmol) at rt. The mixture was heated to 80° C. and Br₂ (3.6 mL, 47.2 mmol) was added dropwise during 40 min. After addition, the mixture was stirred at 80° C. for 3 h. Then the mixture was cooled to rt, and filtered. The filtrate was treated with MgSO₄, filtered and concentrated in vacuo. The residue XVIII-2 (11 g, yield 94.8%) was used in next step directly.

A solution of XVIII-2 (6 g, 24.2 mmol) in anhydrous benzene (60.15 g) was added dropwise to an ice-water cooled suspension of AlCl₃ (5.95 g, 45.1 mmol) in benzene (60.15 g) under nitrogen. The resulting reaction mixture was allowed to stirred in the ice bath for 30 min and then at rt overnight. The mixture was heated to 60° C. for 4 h and then allowed to cool to rt and poured into ice and concentrated HCl. The mixture was extracted with EtOAc, washed with brine, separated, and dried over Na₂SO₄ to leave an orange-brown solid, which was purified by column chromatography (PE:EA=10:1) to afford XVIII-3 (2.3 g, yield: 38.6%).

To a solution of XVIII-4 (1.5 g, 6.52 mmol) in DCM (25 mL) was added DMF (2 drops) followed by oxalyl chloride (1.23 g, 9.78 mmol). The reaction mixture was allowed to stir at rt overnight. The solvent was evaporated under reduced pressure to leave crude XVIII-5 (1.5 g, yield: 92.6%), which was used directly in the next step.

XVIII-5 (1.5 g, 6.05 mmol) was dissolved in a solution of MeCN/THF (v/v=1/1, mL) and added dropwise to an ice water cooled solution of TMSCHN₂ (4.84 mL, 9.68 mmol) and TEA (1.22 g, 12.1 mmol) in a mixture of MeCN and THF (v/v=1/1, 15 mL). The reaction mixture was allowed to stir at 0° C. for 1 h and then for 5 h at rt. The solvent was removed under vacuum and the mixture was diluted with EtOAc and water, and the organic layer was separated, dried and concentrated. The residue was purified by column chromatography (PE:EA=10:1) to afford XVIII-6 (0.4 g, yield: 29.2%). MS (ESI) m/z (M+H)⁺255.2.

XVIII-6 (0.6 g, 2.36 mmol) in methanol (20 mL) and placed in an ultrasound bath, a solution of XVIII-6A (108 mg, 0.47 mmol) in TEA (953 mg, 9.44 mmol) was added dropwise, and the mixture was sonicated for 5 h at rt. The mixture was diluted with EtOAc, washed with brine, dried over Na₂SO₄, filtered and concentrated. The residue was purified by column chromatography (PE:EA=10:1) to afford XVIII-7 (350 mg, yield: 57%).

A chloroform (10 mL) solution of bromine (186 mg, 1.16 mmol) was added dropwise to a vigorously stirred mixture of XVIII-7 (300 mg, 1.16 mmol) and CF₃CO₂Ag (308 mg, 1.39 mmol) in chloroform (10 mL). After stifling for 3 h, the mixture was diluted with EtOAc, washed with brine, dried over Na₂SO₄, filtered and concentrated. The residue was purified by column chromatography (PE:EA=10:1) to afford XVIII-8 (220 mg, yield: 56%).

XVIII-9 and XVIII-10 were prepared following the similar procedures described in the synthesis of III-3 and III-5.

IT026 and its sodium salt IT026a were prepared following the similar procedure described in the synthesis of IT001 and IT001a. IT026: MS (ESI) m/z (M+H)⁺505.1. IT026a: ¹HNMR (DMSO-d₆ 400 MHz) δ7.38-7.51 (m, 9H), 5.72-5.73 (m, 1H), 2.23 (s, 3H), 1.78 (s, 2H), 1.71-1.72 (m, 6H), 1.52-1.65 (m, 6H), 1.51-1.52 (m, 3H). MS (ESI) m/z (M+H)⁺505.2.

IT093 was prepared following the similar synthetic scheme of IT026 using methyl 1-(4-phenylbicyclo[2.2.2]octan-1-yl)cyclopropanecarboxylate in place of XVIII-7. ¹H NMR (CDCl₃ 400 MHz): δ 7.31-7.38 (m, 9H), 6.02 (s, 1H), 5.85 (br, 1H), 2.37 (s, 2H), 1.81-1.83 (m, 6H), 1.71-1.83 (m, 6H), 1.57 (br, 3H), 0.97 (br, 2H), 0.79 (br, 2H). MS (ESI) m/z (M+H)⁺531.2.

### Example 10

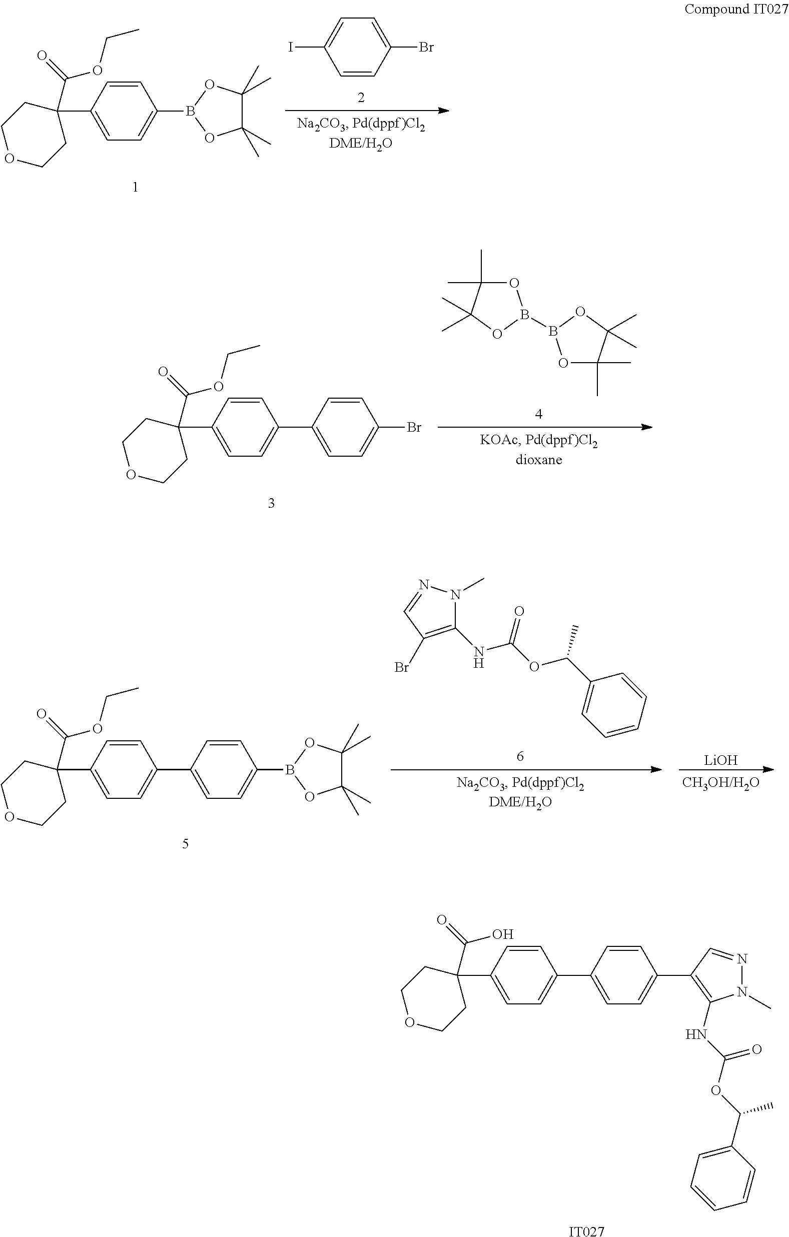

IT027 was prepared from compound 1 as described in the scheme above and followed the similar procedures as described in the synthesis of III-3, 111-5 and IT004. Sodium salt IT027a: ¹H NMR (Methanol-d₄, 400 MHz) δ 7.36-7.73 (m, 14H), 5.8 (m, 1H), 3.88 (d, J=11.8 Hz, 2H), 3.70-3.77 (m, 5H), 2.53 (d, J=13.2 Hz, 2H), 1.89 (t, J=10.0 Hz, 2H), 1.60 (s, 3H). MS (ESI) m/z (M+Na)⁺526.2.

IT042 was prepared following the similar synthetic scheme as IT027, using 1-bromo-2,5-difluoro-4-iodobenzene in place of compound 2 and VI-6A in place of compound 6. Sodium salt IT042a: ¹H NMR (400 MHz, DMSO-d₆): δ7.47-7.57 (m, 7H), 7.26-7.45 (m, 4H), 5.69 (br, 1H), 3.71-3.73 (m, 2H), 3.51-3.56 (m, 2H), 2.43-2.46 (m, 2H), 2.29 (s, 3H), 1.48-1.59 (m, 5H). MS (ESI) m/z (M+H)⁺579.1.

IT044 was prepared following the similar synthetic scheme as IT027, using 1-bromo-2,5-difluoro-4-iodobenzene in place of compound 2. IT044: MS (ESI) m/z (M+H)⁺562.2. Sodium salt IT044a: ¹H NMR (DMSO-d₆, 400 MHz): δ9.76 (s, 1H), 7.73 (s, 1H), 7.51-7.74 (m, 11H), 5.76 (br, 1H), 3.52-3.68 (m, 7H), 2.45-2.46 (m, 2H), 1.67-1.78 (m, 2H), 1.50 (br, 3H). MS (ESI) m/z (M+H)⁺562.2.

IT045 was prepared following the similar procedure for the synthesis of IT042. IT045: MS (ESI) m/z (M+H)⁺565.1. IT045a: ¹H NMR (400 MHz, Methanol-d₄): δ7.61 (d, J=8.0 Hz, 2H), 7.53 (d, J=8.0 Hz, 2H), 7.32-7.40 (m, 7H), 5.16 (s, 2H), 3.87-3.90 (m, 2H), 3.72-3.77 (m, 2H), 2.53-2.57 (m, 2H), 2.38 (s, 3H), 1.86-1.92 (m, 2H). MS (ESI) m/z (M+H)⁺565.1.

### Example 11

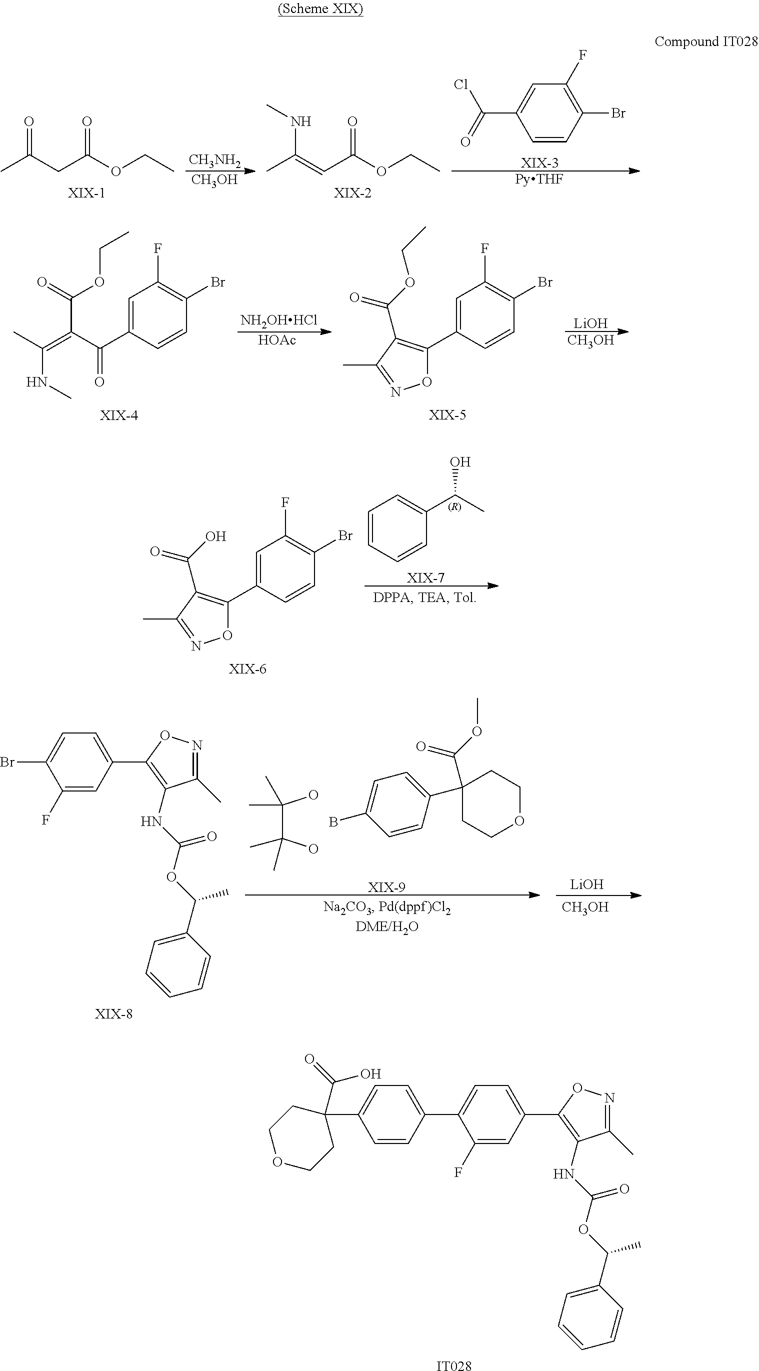

Methylamine solution in MeOH (90.3 g, 768 mmol, 27% w/w) was added into XIX-1 (50 g, 384 mmol) at rt, then the mixture was heated to 45° C. for 18 h. After being cooled to rt., the mixture was extracted with DCM, and the combined organic layer was washed with water, dried over Na₂SO₄, and concentrated in vacuum to give XIX-2 (49 g, yield 89%) without purification.

To a stirred solution of XIX-2 (2.15 g, 13.7 mmol) and pyridine (1.08 g, 13.7 mmol) in THF was added dropwise XIX-3 (3.17 g, 13.7 mmol) at 0° C. under nitrogen. The solution was stirred for 0.5 h, then warmed slowly to rt and stirred overnight. H₂O (20 mL) was added, and the mixture was extracted with EtOAc. The organic layer was combined and washed with brine, dried over Na₂SO₄, concentrated in vacuum to afford XIX-4 (4.5 g, crude yield 95.7%) as a yellow solid, and used in nest step directly.

To a stirred solution of crude XIX-4 (4.5 g, 13.7 mmol) in HOAc (30 mL) was added hydroxylamine hydrochloride (0.95 g, 13.7 mmol) under nitrogen. After the addition, the solution was heated to reflux under nitrogen for 2 h. The solvent was removed under vacuum and the residue was purified by column chromatography on silica gel (PE:EA=5:1) to afford XIX-5 (2.5 g, yield 58%) as a white solid, followed by LiOH hydrolysis in MeOH/H₂O (v/v=5:1) refluxing under nitrogen for 1 h. MeOH was removed in vacuo and the residue was adjusted to pH=2. After standard work-up procedure and purification, XIX-6 (2.0 g, yield 85%) was obtained as a white solid.

The mixture of XIX-6 (1 g, 3.3 mmol), XIX-7 (0.49 g, 4 mmol), DPPA (1.1 g, 4.0 mmol) and Et₃N (0.7 g, 2.6 mmol) in toluene (30 mL) was heated to reflux under nitrogen for 1 h. The mixture was concentrated, and the residue was partitioned between H₂O and DCM. The organic layer was washed with brine, dried over Na₂SO₄, and concentrated. The residue was purified by column chromatography on silica gel (PE:EA=3:1) to afford XIX-8 (0.9 g, yield 65%) as a white solid.

IT028 and its sodium salt IT028a were prepared following the similar procedures for the preparation of III-5, IT001 and IT001a. IT028: MS (ESI) m/z (M+Na)⁺545.2. IT028a: ¹H NMR (Methanol-d₄, 400 MHz): δ7.29-7.61 (m, 12H), 5.80-5.82 (q, 1H), 3.87-3.90 (m, 2H), 3.71-3.77 (m, 2H), 2.54 (d, J=12.4 Hz, 2H), 2.17 (s, 3H), 1.85-1.93 (m, 2H), 1.56 (d, J=5.6 Hz, 3H). MS (ESI) m/z (M+Na)⁺545.2.

IT029 was prepared following the similar procedure for the synthesis of IT028 using 4-chloro-2,5-difluorobenzoyl chloride to replace XIX-3 to afford a yellow solid. Sodium salt IT029a: MS (ESI) m/z (M+H)⁺563.2. ¹H NMR (Methanol-d₄, 400 MHz): δ7.31-7.64 (m, 11H), 5.76-5.78 (q, 1H), 3.89-3.92 (m, 2H), 3.74-3.79 (m, 2H), 2.57 (d, J=12.8 Hz, 2H), 2.25 (s, 3H), 1.88-1.93 (m, 2H), 1.56 (d, J=12.8 Hz, 3H).

### Example 12

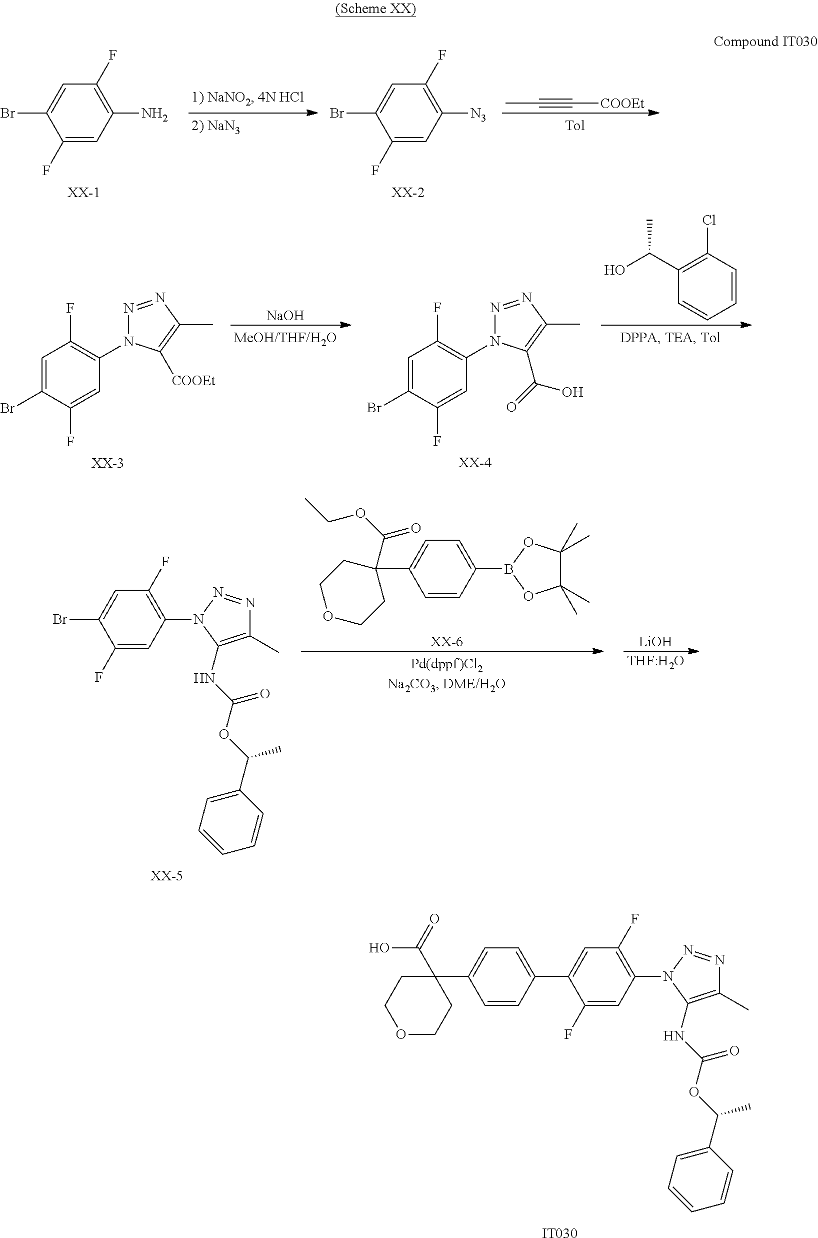

To a solution of XX-1 (5 g, 24 mmol) in 4N hydrochloride solution (36 mL) was added dropwise of NaNO₂ (1.84 g, 26.7 mmol) in water (10 mL) at 0° C. After addition, the mixture was stirred for 30 minutes, then NaN₃ (1.89 g, 29.3 mmol) was added. The reaction mixture was slowly warmed to rt and stirred for 1 h. The reaction mixture was extracted with MTBE. The combined organic phase was dried over Na₂SO₄, filtered and concentrated to give crude XX-2 (5.63 g, crude yield: 100%), which was used to next step directly.

To a solution of XX-2 (5.63 g, 24.27 mmol) in toluene (50 mL) was added But-2-ynoic acid ethyl ester (3.36 mL, 29.1 mmol). The reaction mixture was flushed with nitrogen and heated to reflux overnight. The reaction mixture was concentrated, and the residue was purified by column chromatography (PE:EA=5:1) to give XX-3 (6 g, yield: 71.5%).

To a solution of XX-3 (1 g, 2.89 mmol) in MeOH/THF/H₂O (10 mL/10 mL/10 mL) was added NaOH (578 mg, 14.45 mmol). The reaction mixture was stirred at rt overnight. The mixture was cooled down to 0° C. and neutralized to pH=4.0 with 3N HCl. The mixture was extracted with EtOAc, dried over Na₂SO₄ and concentrated to give crude XX-4 (659 mg, yield: 71.7%), which was used to next step directly.

To a solution of XX-4 (459 mg, 1.627 mmol) in dry toluene (5 mL) was added (R)-1-phenylethanol (535 mg, 1.95 mmol), TEA (238 mg, 3.25 mmol) and DPPA (451 mg, 1.95 mmol). The reaction mixture was heated to 80° C. for 3 h. The mixture was diluted with EtOAc, washed with brine, dried over Na₂SO₄, filtered and concentrated. The residue was purified by column chromatography (PE:EA=10:1) to give XX-5 (490 mg, yield: 97%). MS (ESI) m/z (M+H)⁺438.0.

IT030 and its sodium salt IT030a were prepared following the similar procedures for the preparation of III-5, IT001 and IT001a. IT030: MS (ESI) m/z (M+H)⁺563.2. IT030a: ¹H NMR (DMSO-d₆, 400 MHz): δ7.65 (d, J=8.4 Hz, 2H), 7.37-7.54 (m, 3H), 7.32-7.36 (m, 1H), 7.34-7.19 (m, 5H), 5.72-5.67 (q, 1H), 3.81-3.91 (m, 2H), 3.73-3.77 (m, 2H), 2.56 (d, J=12.4 Hz 2H), 2.26 (s, 3H), 1.87-1.93 (m, 2H), 1.47 (br, 3H). MS (ESI) m/z (M+H)⁺563.2.

IT072 was prepared following the general synthetic scheme for the synthesis of IT030, using 4-bromoaniline in place of XX-1, F₃C≡COOEt in place of But-2-ynoic acid ethyl ester, and

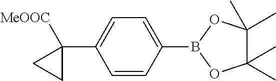 [SMILES: COC(=O)C1(c2ccc(B3OC(C)(C)C(C)(C)O3)cc2)CC1]

in place of XX-6. MS (ESI) m/z (M+H)⁺537.2. ¹H NMR (Methanol-d₄, 400 MHz): δ7.75 (d, J=8.0 Hz, 2H), 7.63 (d, J=8.0 Hz, 2H), 7.49-7.55 (m, 4H), 7.21-7.26 (m, 5H), 5.69 (q, J=6.0 Hz, 1H), 1.63-1.65 (m, 2H), 1.47 (br, 3H), 1.26-1.28 (m, 2H).

IT075 was prepared following the general synthetic scheme for the synthesis of IT030, using

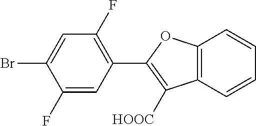 [SMILES: O=C(O)c1c(-c2cc(F)c(Br)cc2F)oc2ccccc12]

in place of XX-4 and

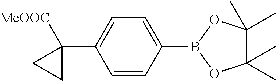 [SMILES: COC(=O)C1(c2ccc(B3OC(C)(C)C(C)(C)O3)cc2)CC1]

in place of XX-6. MS (ESI) m/z (M+Na)⁺576.0. Sodium salt IT075a: ¹H NMR (DMSO-d₆, 400 MHz): δ 9.73 (brs, 1H), 7.32-7.65 (m, 15H), 5.74-5.76 (m, 1H), 1.55 (br, 3H), 1.26 (br, 2H), 0.79 (br, 2H). MS (ESI) m/z (M+Na)⁺576.0.

### Example 13

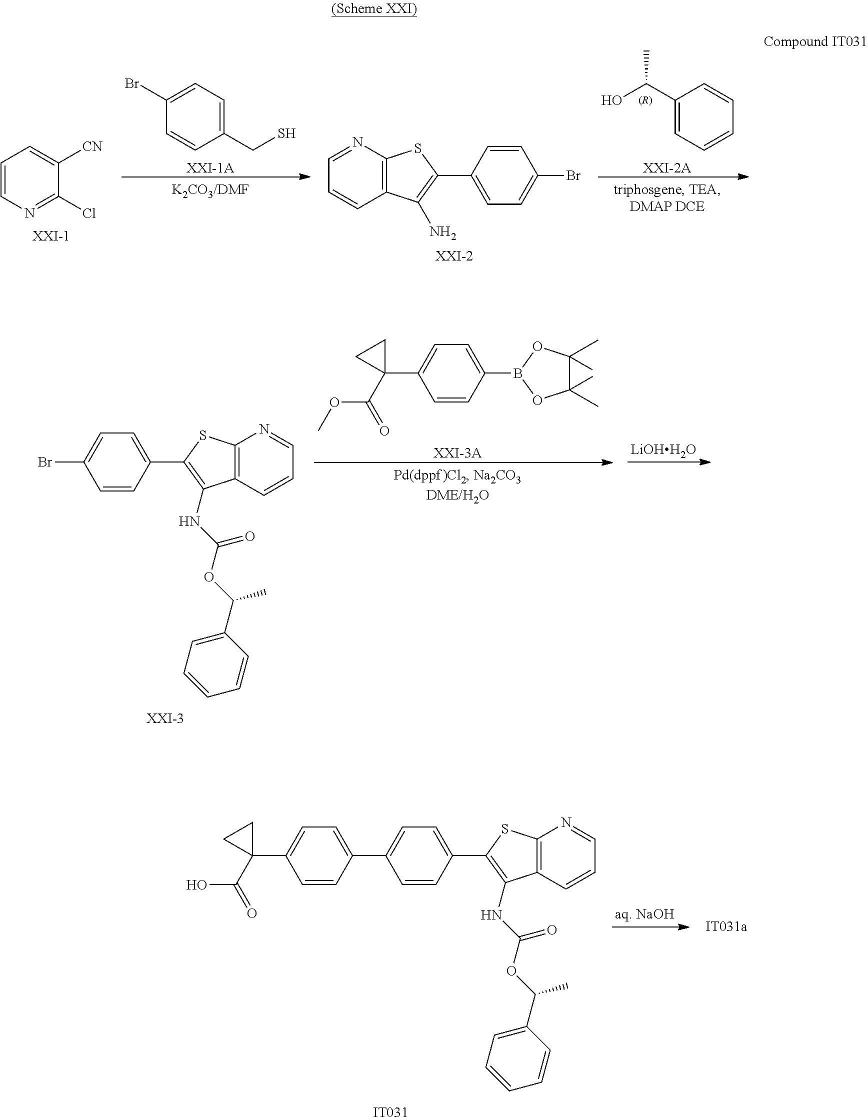

A mixture of XXI-1 (2.5 g, 18.04 mmol), XXI-1A (3.66 g, 18.04 mmol) and K₂CO₃ (9.98 g, 72.16 mmol) in 40 mL DMF was heated to 80° C. and stirred overnight. Then the reaction mixture was heated at 130° C. and stirred for additional 18 h. After cooled to rt., the mixture was diluted with water, extracted with EtOAc. The combined organic layer was washed with brine, dried and concentrated. The resulting solid was washed with tert-butylmethylether to afford XXI-2 (3.5 g, yield 64%).

To a solution of XXI-2 (1 g, 3.28 mmol), TEA (2.3 mL, 16.4 mmol) and DMAP (1.49 g, 3.28 mmol) in 50 mL of dichloroethane was added triphosgene (0.97 g, 3.28 mmol) at 0° C. Then XXI-2A (2 g, 16.38 mmol) was added. The reaction mixture was stirred for 1 hour. The mixture was diluted with DCM, washed with H₂O, brine, dried and concentrated. The residue was purified by flash column chromatography over silica gel (PE:EA=4/1) to afford XXI-3 (1.1 g, yield 73%).

IT031 and its sodium salt IT031a were prepared following the similar procedures for the preparation of III-5, IT001 and IT001a. IT031a: ¹HNMR (400 MHz, DMSO-d₆) δ 9.61 (br, 1H), 8.60 (d, J=3.6 Hz, 1H), 7.91-7.92 (m, 1H), 7.68-7.72 (m, 4H), 7.36-7.56 (m, 10H), 5.75-7.56 (m, 1H), 1.55-1.56 (m, 3H), 1.22-1.23 (m, 2H), 0.72-0.73 (m, 2H). MS (ESI) m/z (M+H)⁺ 535.3.

### Example 14

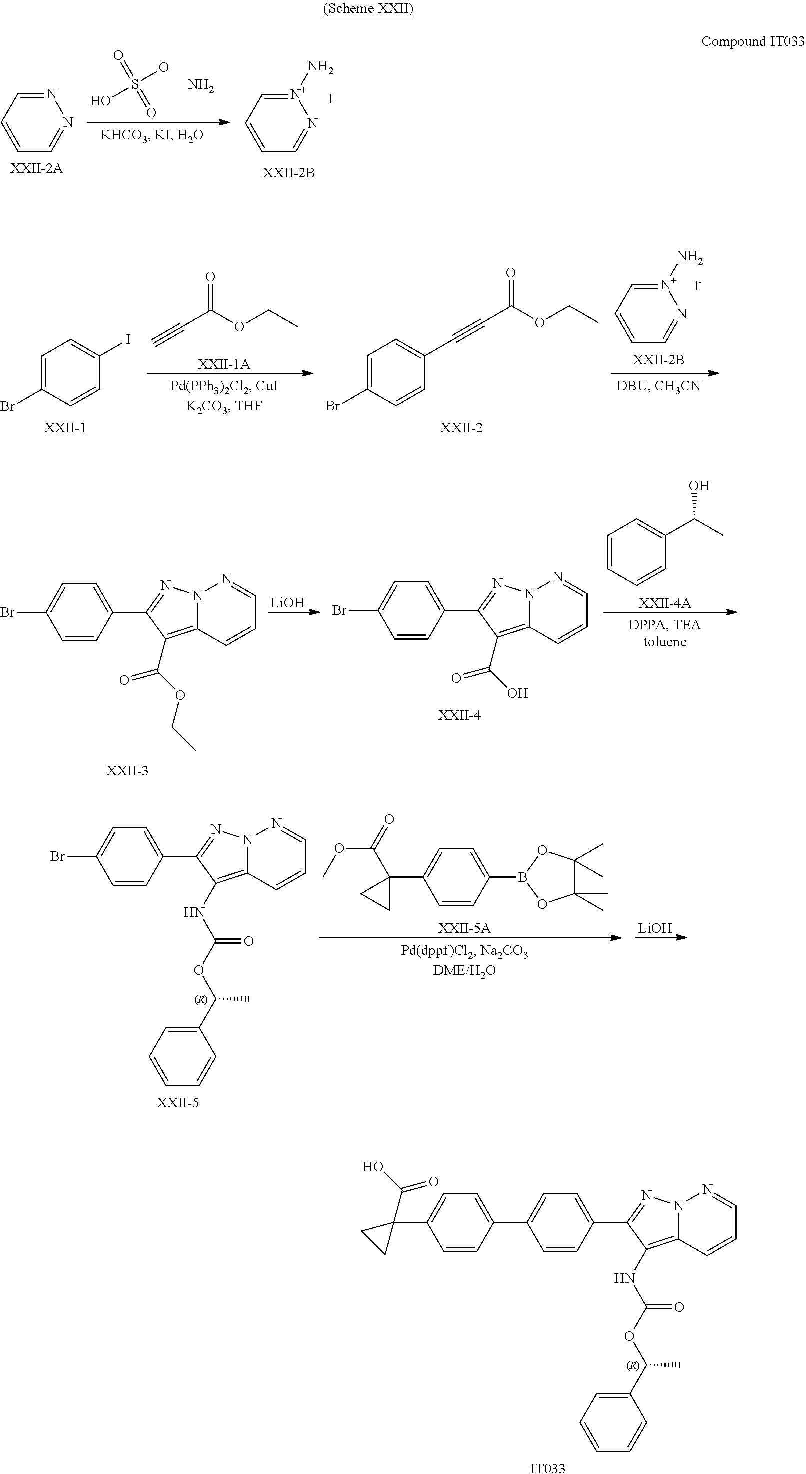

Aqueous KHCO₃ (2.4 mmol/mL) was added to a solution of Hydroxylamine-O-sulfonic acid (4.28 g, 37.9 mmol) in H₂O (8 mL) was cooled to 10° C. until pH to 5.0. Then XXII-2A (2 g, 25 mmol) was added in one portion and the reaction mixture was heated to 70° C. for 1 h. The pH was adjusted to 7.0 by the addition of aq. KHCO₃. The reaction was cooled to 40° C. and the mixture was allowed to stir for 1 h. Then KI (4.12 g, 25 mmol) in H₂O (8 mL) was added, and the solvent was removed in vacuo, followed by the addition of 5% methanol in ethanol (20 mL). The solids were collected by filtration and dried in vacuo to give crude XXII-2B (3.5 g, yield: 63.6%), which was used to next step directly.

To a solution of XXII-1 (10 g, 35.5 mmol) in THF (200 mL) was added K₂CO₃ (9.8 g, 71.0 mmol), CuI (270 mg, 1.42 mmol), Pd(PPh₃)₂Cl₂ (496 mg, 0.708 mmol) and XXII-1A (13.8 g, 140.8 mmol). The mixture was heated at 70° C. under N₂ for 12 h. After cooled to rt, water (50 mL) was added, and extracted with EtOAc. The organic layer was separated, dried, and concentrated, and the residue was purified by column chromatography (PE:EA=30:1) to afford XXII-2 (2.8 g, yield: 31.2%).

DBU (0.59 mL, 7.9 mmol) was added dropwise to a solution of XXII-2 (1 g, 3.95 mmol) and XXII-2B (1.76 g, 7.9 mmol) in CH₃CN (20 mL). The resulting mixture was stirred at 25° C. for 12 h. The solvent was removed under reduced pressure. The residue was dissolved in EtOAc and washed with H₂O. The organics were collected, dried with Na₂SO₄, filtered, and concentrated. The residue was purified by column chromatography (PE:EA=3:1) to give XXII-3 (148 mg, yield: 10.8%).

To a stirred solution of XXII-3 (148.0 mg, 0.4 mmol) in 15 mL of MeOH/H₂O/THF (v/v/v=1/1/1) was added LiOH.H₂O (90 mg, 2.2 mmol). After the addition, the solution was stirred at rt for 12 h. The mixture was concentrated in vacuo and adjusted pH to 4 with HCl (1N). The aqueous phase was extracted with EtOAc, washed with brine, dried over Na₂SO₄, and concentrated to afford crude XXII-4 (120.0 mg, yield 87.5%), which was used to next step directly.

The mixture of XXII-4 (220 mg, 0.69 mmol), XXII-4A (101 mg, 0.83 mmol), DPPA (228 mg, 0.83 mmol) and TEA (139 mg, 1.38 mmol) in toluene (10 mL) was stirred at 80° C. under nitrogen for 12 h. After cooled to rt, water was added. The organic layer was extracted with EtOAc, separated, dried, and concentrated. The residue was purified by chromatography on silica gel (PE: EA=1:1) to afford XXII-5 (156 mg, yield: 53.1%).

IT033 and its sodium salt IT033a were prepared following the similar procedures for the preparation of III-5, IT001 and IT001a. IT033: MS (ESI) m/z (M+H)⁺519.1. IT033a: ¹HNMR (DMSO-d₆, 400 MHz): δ 9.37 (s, 1H), 8.47 (s, 1H), 7.94-8.00 (m, 3H), 7.69-7.71 (m, 2H), 7.22-7.55 (m, 10H), 5.78 (q, 1H), 1.56 (d, J=5.2 Hz, 3H), 1.21 (br, 2H), 0.71 (br, 2H). MS (ESI) m/z (M+H)⁺519.1.

IT049 was prepared following the similar synthetic scheme of IT033 using 1-bromo-2,5-difluoro-4-iodobenzene in place of XXII-1 and ethyl 2-(4-bromo-2,5-difluorophenyl)pyrazolo[1,5-a]pyridine-3-carboxylate in place of XXII-3. In the last step Suzuki-coupling reaction, x-Phos and Pd₂(dba)₃ in dioxane were used instead of Pd(dppf)Cl₂ in DME/H₂O. IT049: MS (ESI) m/z (M+H)⁺554.18. Sodium salt IT049a: ¹H NMR (DMSO-d₆, 400 MHz): δ 9.15 (s, 1H), 8.70 (d, J=6.8 Hz, 1H), 7.27-7.53 (m, 13H), 0.97-0.99 (m, 1H), 5.72-5.74 (m, 1H), 1.51 (d, J=5.6 Hz, 2H), 1.17 (br, 3H), 0.71 (br, 2H). MS (ESI) m/z (M+H)⁺554.1.

IT061 was prepared following the similar synthetic scheme of IT033 using 1-bromo-2,5-difluoro-4-iodobenzene in place of XXII-1. IT061: MS (ESI) m/z (M+H)⁺555.1. Sodium salt IT061a: ¹HNMR (DMSO-d₆, 400 MHz): δ 9.40 (s, 1H), 8.52 (s, 1H), 8.11 (br, 1H), 7.25-7.58 (m, 12H), 5.74-5.75 (m, 1H), 1.52 (d, J=6.4 Hz, 3H), 1.26 (br, 2H), 0.80 (br, 2H). MS (ESI) m/z (M+H)⁺555.1.

### Example 15

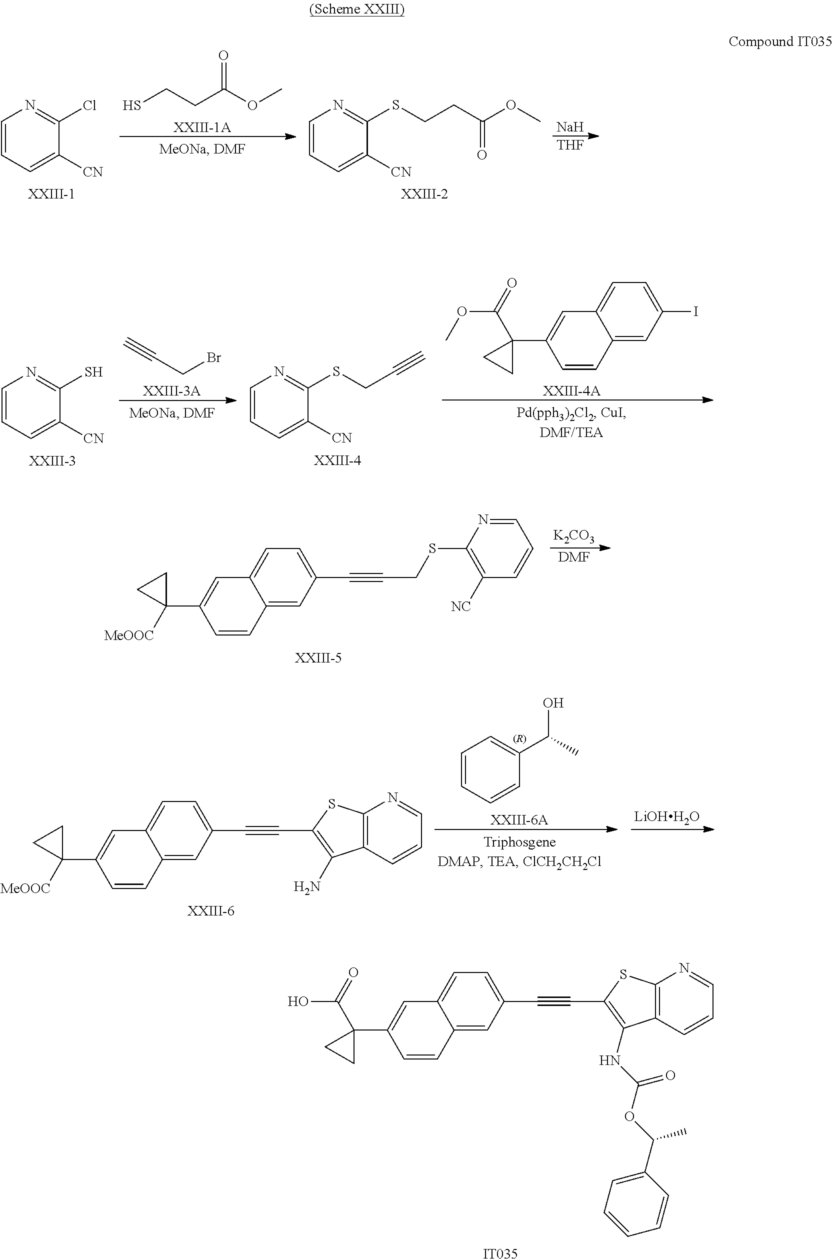

A mixture of sodium methoxide (3.48 g, 0.065 mol), XXIII-1A (18 g, 0.15 mol) and XXIII-1 (20.7 g, 0.15 mol) in dry DMF (30 mL) was stirred at rt for 24 hs, The mixture was poured into water and extracted with EA. The organic layer was washed with water, dried over Na₂SO₄, filtered, and evaporated to dryness. The residue was purified to afford XXIII-2 (9.0 g, yield 27%).

A mixture of XXIII-2 (3.0 g, 13.514 mmol), NaH (60%, 1.622 g) in THF (60 mL) was stirred at refluxed for 5 hs. After being cooled to rt, the excess hydride was destroyed by the addition of ice/water (5 mL). The solvent was removed in vacuo, and neutralized to pH=6.0 with 1N HCl. The precipitated solids was filtered and purified by prep-HPLC to afford XXIII-3 (1.2 g, yield 65.3%).

A solution of XXIII-3 (420 mg, 3.088 mmol) in dry DMF (5 mL) was treated with fresh sodium methoxide (183 mg, 3.397 mmol) at 0° C. Then XXIII-3A (400.8 mg, 3.397 mmol) in dry DMF (0.5 mL) was added dropwise to the mixture. The resulting mixture was stirred at rt overnight. The mixture were poured into water and extracted with EA. The organic layer was washed with water, dried over Na₂SO₄, filtered, and evaporated to dryness. The residue was purified by column chromatography (PE:EA=30:1) to give XXIII-4 (470 mg; yield: 87.5%).

XXIII-4A (811 mg, 2.241 mmol), Pd(PPh₃)₂Cl₂ (60.4 mg, 0.086 mmol), and CuI (32.8 mg, 0.172 mmol) were mixed with DMF (3 mL) and freshly distilled TEA (9 mL). Then a solution of the XXIII-4 (300 mg, 1.724 mmol) in DMF/TEA (3 mL/9 mL) was added slowly over the course of 1 h at rt. Once the addition is completed, TLC showed complete reaction. The mixture was poured into water, and extracted with EA. The extraction was washed with brine, dried over Na₂SO₄, filtered, and evaporated to dryness. The residue was purified by column chromatography (PE:EA=15:1) to give XXIII-5 (350 mg, yield: 51%).

To a solution of XXIII-5 (350 mg, 0.879 mmol) in dry DMF (4 mL) was added K₂CO₃ (485 mg, 3.518 mmol) at rt. The mixture was heated to 60° C. for 8 hs. The mixture was poured into water and extracted with EA. The extraction was washed with brine, dried over Na₂SO₄, filtered, and evaporated to dryness. The residue purified by column chromatography (PE:EA=15:1) to afford XXIII-6 (180 mg, yield: 51.4%).

To a solution of XXIII-6 (90 mg, 0.226 mmol) in 1,2-dichloroethane (2 mL) was added DMAP (27.6 mg, 0.226 mmol) and TEA (114 mg, 1.130 mmol). The mixture was stirred at 0° C. for 15 min., and then triphosgene (67 mg, 0.226 mmol) was added to the brown solution at 0° C. The mixture was stirred for 10 min, XXIII-6A (27.6 mg, 0.226 mmol) in 1,2-dichloroethane (1 mL) was added, and the reaction mixtures was stirred at rt for 2 h under N₂. The mixture was poured into water and extracted with EA. The organic layer was washed water, dried over Na₂SO₄, filtered, and evaporated to dryness. The residue was purified and further subject to hydrolysis by LiOH.H₂O (22.6 mg, 0.94 mmol at rt overnight. The mixture was poured into water, neutralized to pH=6.0, then extracted with EA. The organic layer was washed water, dried over Na₂SO₄, filtered, and evaporated to dryness. The residue was purified by preparative HPLC to afford IT035 (56 mg, yield 56.8%).

To a solution of IT035 (58.6 mg, 0.104 mmol) in MeOH/H₂O (v/v=3/1, 5 mL) was added aq. NaOH (2.48 mL, 0.05N, 0.104 mmol) at 0° C. The reaction mixture was stirred for 30 minutes. The reaction mixture was lyophilized to give IT035a. ¹H NMR (400 MHz, DMSO-d₆): 9.97 (s, 1H), 8.66 (dd, J=4.4 Hz, J=1.2 Hz, 1H), 8.01-8.08 (m, 2H), 7.85 (d, J=8.4 Hz, 1H), 7.67-7.77 (m, 2H), 7.57 (d, J=8.4 Hz, 1H), 7.34-7.48 (m, 6H), 7.27-7.33 (m, 1H), 5.87 (q, J=6.53 Hz, 1H), 1.58 (d, J=6.4 Hz, 3H), 1.22-1.27 (m, 2H), 0.77-0.82 (m, 2H). MS (ESI) m/z (M+H)⁺533.3.

### Example 16

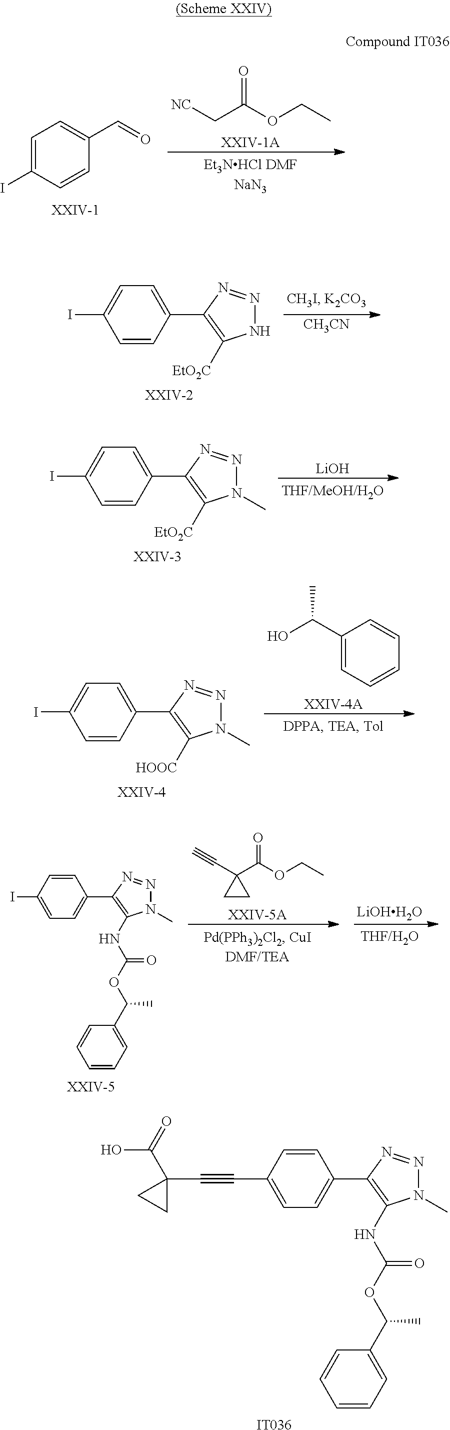

To a solution of XXIV-1 (12 g, 51.7 mmol) in DMF (180 mL) were added Et₃N.HCl (21.3 g, 155.1 mmol), NaN₃ (10.3 g, 163.5 mmol) and XXIV-1A (5.84 g, 51.7 mmol). The reaction mixture was heated at 70° C. for 18 hours under nitrogen protection. After completion of the reaction, the mixture was poured into water and extracted with EtOAc. The organic layers were dried over MgSO₄ and concentrated. The residue was purified by chromatography on silica gel (PE:EA=5:1) to afford XXIV-2 (6 g, yield: 33.8%).

To a solution of XXIV-2 (3 g, 8.752 mmol) in CH₃CN (50 mL), K₂CO₃ (2.41 g, 17.5 mmol), was added MeI (2.5 g, 17.5 mmol). The reaction mixture was stirred at rt overnight under nitrogen protection. Then CH₂Cl₂ and water was added, the organic layers were separated, dried over MgSO₄ and concentrated. The residue was purified by prep-HPLC to afford XXIV-3 (0.5 g, yield: 16.1%).

To a stirred solution of XXIV-3 (4.2 g, 14.2 mmol) in MeOH/THF/H₂O (v/v/v=1/2/1, 16 mL) was added LiOH (3 g, 71 mmol). After the addition, the solution was stirred overnight at rt. The solution was concentrated in vacuo, the aqueous layer was adjusted pH to 2, and extracted with EtOAc. The organic layer was separated, dried and concentrated to afford XXIV-4 (0.7 g, yield? 6%).

To a solution of XXIV-4A (236 mg, 1.93 mmol) in dry toluene (8 mL) was added XXIV-4 (530 mg, 1.61 mmol), TEA (0.447 mL, 3.22 mmol) and DPPA (0.414 mL, 1.93 mmol). The reaction mixture was heated to 80° C. for 3 hours. The mixture was diluted with EtOAc, washed with brine, dried over Na₂SO₄, filtered and concentrated. The residue was purified by TLC (PE:EA=2:1) to give XXIV-5 (550 mg, yield: 76.1%).

IT036 was prepared from XXIV-5 in two steps following the similar procedure described the synthesis of IT001. MS (ESI) m/z (M+H)⁺431.1. Sodium salt IT036a: ¹HNMR (Methanol-d₄, 400 MHz) δ7.66 (d, J=7.2 Hz, 2H), 7.36-7.45 (m, 9H), 5.84 (q, 1H), 3.91 (s, 3H), 1.57-1.64 (m, 5H), 1.43 (q, 2H). MS (ESI) m/z (M+H)⁺ 431.1.

### Example 17

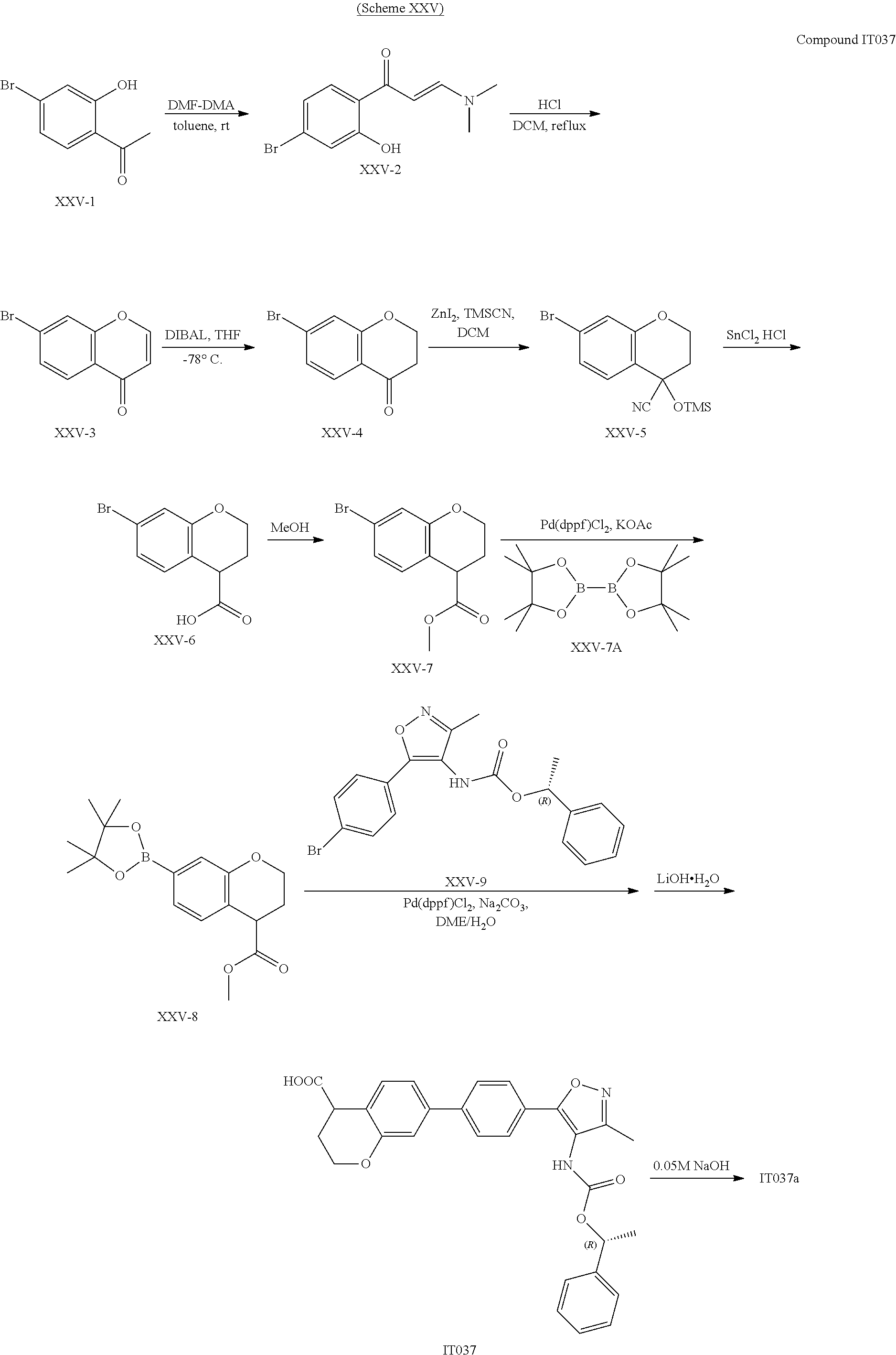

A solution of XXV-1 (5 g, 26 mmol) and DMF-DMA (12 mL, 52 mmol) in toluene (120 mL) was stirred at reflux for 4 hs. The mixture was concentrated and the residue was purified by flash chromatography (PE/EA=10/1) to give XXV-2 (2.7 g, yield: 40%) as a white solid. A solution of XXV-2 (4.5 g, 16.7 mmol) and conc. HCl (5 mL) in DCM (20 mL) was stirred at refluxed for 40 min. The organic layer was separated and the aqueous layer was extracted with DCM, the combined organic layer was washed with NaHCO₃ and brine, dried over Na₂SO₄ and concentrated to give XXV-3 (3.2 g, yield: 86%) as a white solid.

A solution of XXV-3 (2.9 g, 12.9 mmol) in THF (10 mL) was cooled to −78° C., DIBAl-H (24 mL, 24 mmol) was added and the resulting solution was stirred at −78° C. for 30 min., Then sat.NH₄Cl was added to quench the reaction, extracted with EA, washed with brine, dried over Na₂SO₄ and concentrated, the residue was purified by flash chromatography (PE/EA=1/1) to give XXV-4 (1.5 g, yield: 51.7%) as a yellow solid.

To a solution of XXV-4 (2.27 g, 10 mmol) was added the iodine (76.2 mg, 0.3 mmol), TMSCN (1.5 g, 15 mmol) in DCM (20 mL). The resulting solution was stirred at rt for another 24 hs. Then the NaHSO₃ (aq.) was added and extracted with DCM. The organic lays was evaporated in vacuum to afford the crude XXV-5, which was used to next step without purification.

To a solution of XXV-5 (3.27 g, 10 mmol) in HCl/HOAc (v/v=10 mL: 10 mL) was added SnCl₂ (6.6 g, 35 mmol) and the resulting solution was stirred at 80° C. for 18 hs. Then 10 mL of water was added and extracted with DCM. The combined organic layer was washed with 2N NaOH (aq.) and combined the aqueous lays. The aqueous lays was adjusted to pH (<3) with 5N HCl and extracted with DCM, and the combined organic layer was evaporated in vacuum to afford the crude XXV-6 without purification for next step.

To a solution of XXV-6 (1.88 g) in MeOH (20 mL) was added HCl (200 mg) and the resulting solution was stirred at 80° C. for another 4 hs. Then the solvent was evaporated and 50 mL of EA was added and washed with brine. The organic phase was dried over Na₂SO₄ and evaporated. The residue was purified by column chromatography to afford XXV-7 (1.5 g, yield: 76%).

XXV-8 was prepared from XXV-7 and XXV-7A following the similar procedure described in the synthesis of III-3.

IT037 was prepared from XXV-8 and XXV-9 in two steps following the similar procedure described the synthesis of III-5 and IT001. ¹H NMR (DMSO-d₆, 400 MHz): δ 12.62-12.91 (m, 1H), 9.11-9.51 (m, 1H), 7.71-7.88 (m, 5H), 7.38-7.52 (m, 4H), 7.31-7.37 (m, 2H), 7.21-7.29 (m, 1H), 7.11-7.17 (m, 1H), 5.49-5.94 (m, 1H), 4.07-4.45 (m, 3H), 4.07-4.45 (m, 3H), 3.76-3.93 (m, 1H), 2.01-2.29 (m, 7H), 1.45-1.70 (m, 3H). MS (ESI) m/z (M+H)⁺499.1. IT037a: ¹H NMR (Methanol-d₄, 400 MHz): δ 7.58-7.95 (m, 4H), 7.32-7.50 (m, 5H), 7.02-7.23 (m, 3H), 5.73-6.03 (m, 1H), 4.35-4.56 (m, 1H), 4.09-4.32 (m, 1H), 3.62-3.82 (m, 1H), 2.20 (s, 5H), 1.26-1.76 (m, 3H). MS (ESI) m/z (M+H)⁺499.1.

### Example 18

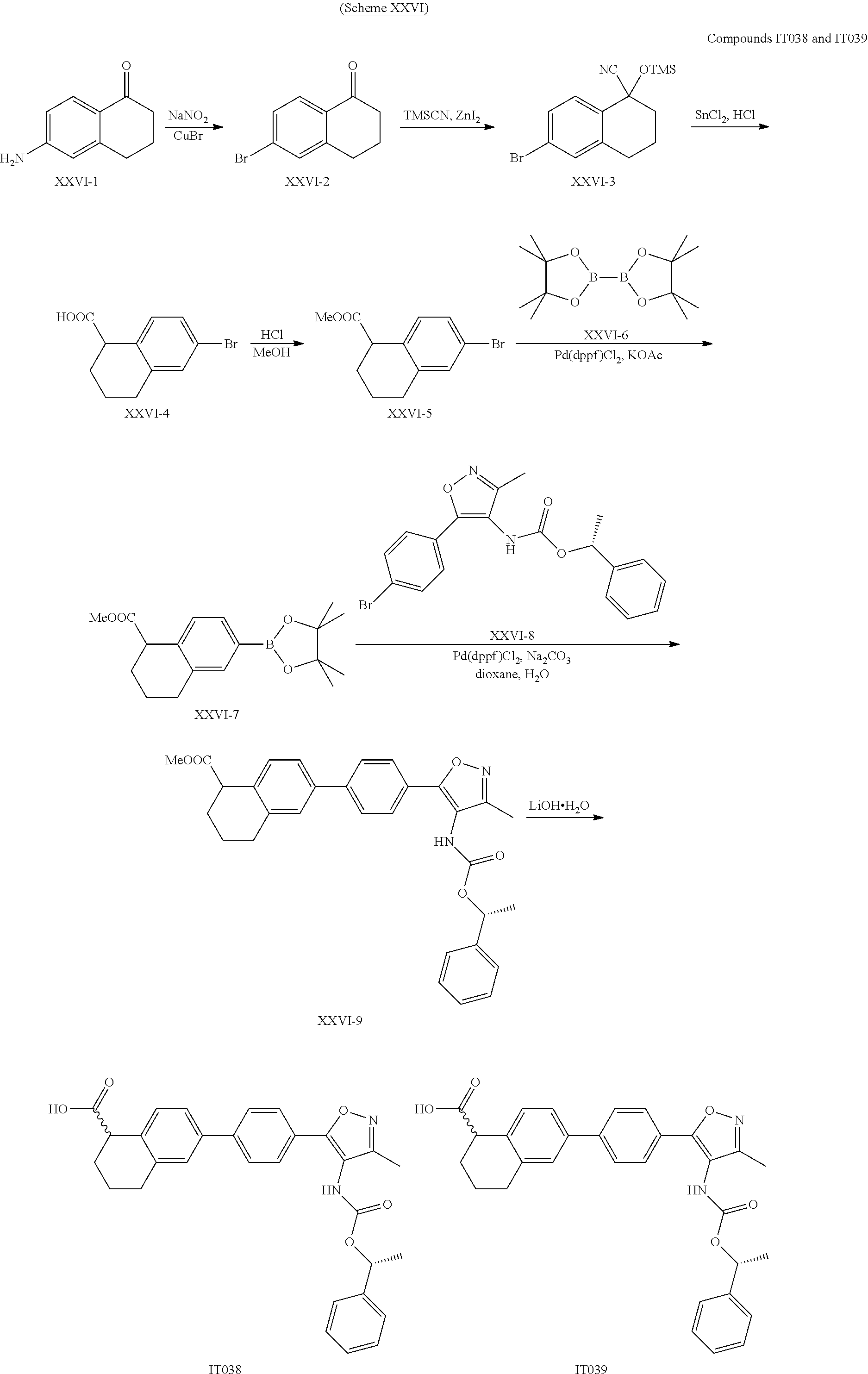

To a stirred solution of XXVI-1 (5.00 g, 0.03 mmol) in 15 mL of 40% aqueous HBr was added a solution of NaNO₂ (2.35 g, 0.034 mmol) in H₂O, maintaining the temperature at −5° C. under nitrogen. After the addition, the solution was stirred for another 0.5 hour. Then the resulting solution was warmed slowly to the rt and stirred for another 3 hours. Then the solution was concentrated and the mixture was extracted with EtOAc. The organic layer was combined and washed with brine, dried over Na₂SO₄, concentrated in vacuo. The residue was purified by column chromatography on silica gel to afford XXVI-2 (3.5 g, yield: 52%).

The solution of XXVI-2 (3.50 g, 15.56 mmol), TMSCN (2.33 g, 23.34 mmol) and I₂ (0.40 g, 1.56 mmol) in DCM (30 mL) was stirred overnight at 25° C. under nitrogen. 20 mL of aqueous Na₂SO₃ was added, and the mixture was extracted with DCM. The organic layer was combined and washed with brine, dried over Na₂SO₄, concentrated in vacuo to afford 3.4 g of crude XXVI-3, which was used for next step without further purification.

XXVI-3 (3.4 g, 15.1 mmol) and SnCl₂ (10.0 g, 52.8 mmol) were added to a solution of HOAc and HCl (10 mL, V/V=1/1) under nitrogen. After the addition, the solution was heated to 90° C. under nitrogen for 24 hours. The mixture was extracted with DCM. The combined aqueous layers were washed with 2M NaOH. The combined aqueous layers were adjusted to pH=2 with 5 M HCl solution (10 mL). The acidic aqueous phase was extracted with EtOAc. The combined organic layer was washed with brine, dried over Na₂SO₄, concentrated to afford XXVI-4 (0.85 g, yield: 22%).

The solution of XXVI-4 (1.13 g, 5.02 mmol) and HCl (13.9 mg, catalyzed amount) in 10 mL of MeOH was heated to reflux under nitrogen for overnight. MeOH was removed in vacuo and the residue was partitioned between H₂O (20 mL) and EtOAc. The organic layer was washed with brine, dried over Na₂SO₄, and concentrated. The residue was purified by column chromatography on silica gel to afford XXVI-5 (0.80 g, yield: 55%).

XXVI-7 was prepared from XXVI-5 and XXVI-6 following the similar procedure described in the synthesis of III-3.

XXVI-7 was prepared from XXVI-7 and XXVI-8 following the similar procedure described the synthesis of III-5.

IT038 and IT039 racemic mixture: MS (ESI) m/z (M+H)⁺498.1. Their sodium salts IT038a and IT039a were obtained from SFC separation. IT038a: ¹H NMR (DMSO-d₆, 400 MHz): δ 9.39 (s, 1H), 7.74-7.79 (m, 4H), 7.33-7.44 (m, 8H), 5.76 (br, 1H), 2.73-2.76 (m, 2H), 2.08-2.13 (m, 4H), 1.96 (s, 1H), 1.76 (s, 1H), 1.56 (s, 3H). MS (ESI) m/z (M+H)⁺498.1. IT039a: ¹H NMR (Methanol-d₄, 400 MHz): δ7.60-7.63 (m, 4H), 7.22-7.33 (m, 8H), 5.71 (br, 1H), 3.58-3.62 (m, 1H), 2.70-2.83 (m, 2H), 1.93-2.07 (m, 6H), 1.61-1.64 (m, 1H), 1.51 (s, 1H). MS (ESI) m/z (M+H)⁺498.1.

### Example 19

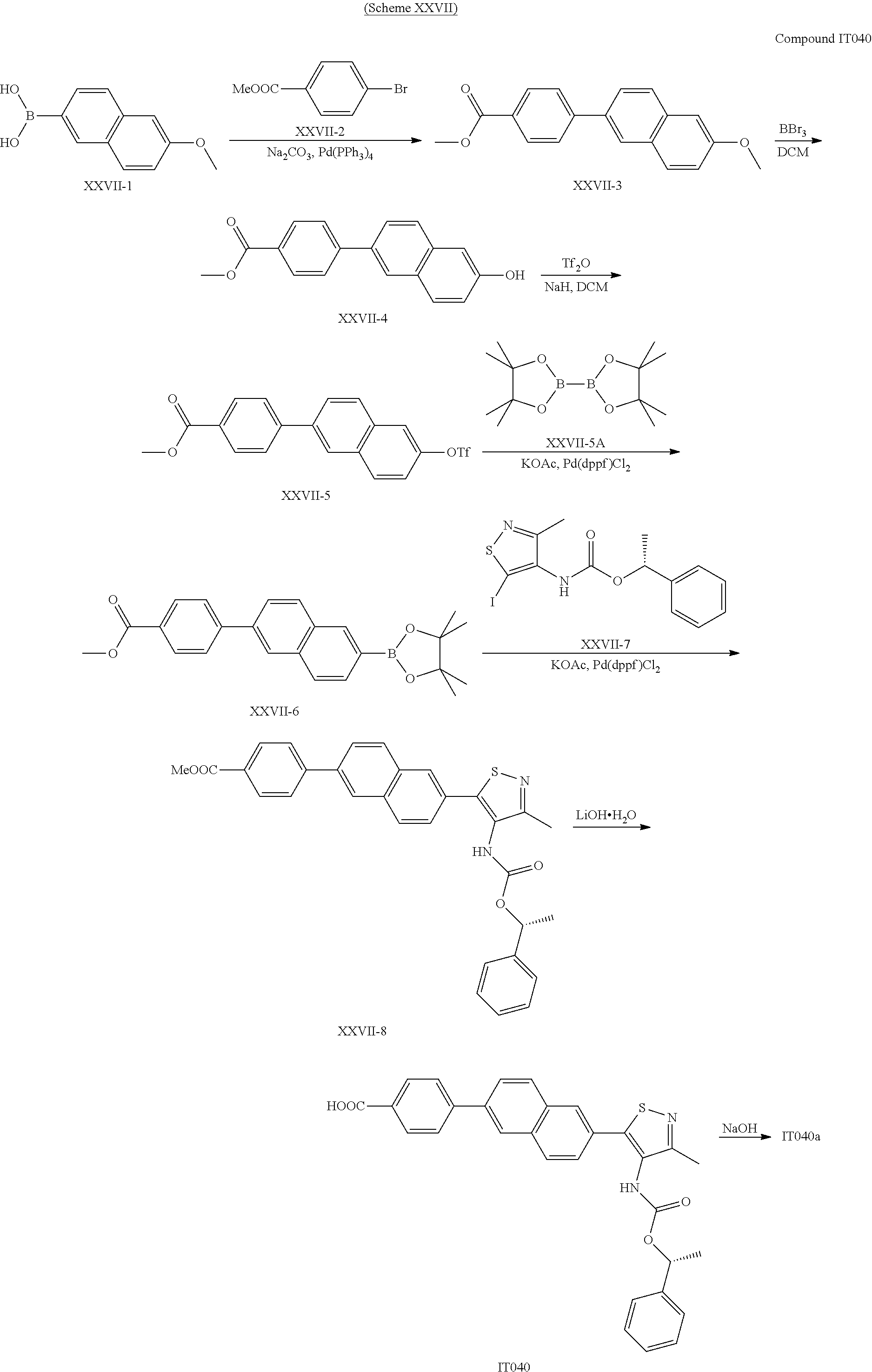

To a stirred solution of XXVII-1 (5.3 g, 26 mmol), XXVII-2 (5 g, 22 mmol), Na₂CO₃ (5.8 g, 55 mmol) in DME/H₂O (60 mL, v/v=5/1) was added Pd(PPh₃)₄ (1.27 g, 1.1 mmol) under nitrogen. Then the solution was heated to 110° C. for overnight. The solid formed was filtered and washed with water and dried in vacuo to obtain XXVII-3 (10 g, crude yield: 100%) as a brown solid.

To a stirred solution of XXVII-3 (300 mg, 1.03 mmol) in DCM (5 mL) was added BBr₃ (1 g, 4.1 mmol) dropwise at −78° C. Then it was stirred at rt for 6 hours. The mixture was quenched with H₂O. The organic layers were washed with brine, and concentrated under vacuo to give XXVII-4 (80 mg, yield: 28%).

To a stirred solution of XXVII-4 (300 mg, 1.08 mmol) in DCM (10 mL) was added NaH (129.6 mg, 3.24 mmol) under nitrogen at 0° C. Then the solution was warmed to rt. After 2 hours, Tf₂O (338 mg, 1.18 mmol) was added, and the mixture was stirred overnight. A saturated solution of NH₄Cl was added. The aqueous phase was extracted with DCM. The organic layer was combined and washed with brine, dried over Na₂SO₄, concentrated in vacuo to afford XXVII-5 (700 mg, crude).

XXVII-6 and XXVII-8 were prepared following the similar procedure in the synthesis of III-3 and III-5.

IT040 and IT040a were prepared following the similar procedure in the synthesis of IT001 and IT001a. IT040: MS (ESI) m/z (M+H)⁺509.1. ¹H NMR (Methanol-d₄, 400 MHz): δ 8.15-8.23 (m, 3H), 7.96-8.06 (m, 2H), 7.91 (d, J=7.6 Hz, 3H), 7.61 (br, 2H), 7.28-7.36 (m, 4H), 6.96 (br, 1H), 5.77 (br, 1H), 2.36 (s, 3H), 1.55 (s, 3H). IT040a: MS (ESI) m/z (M+H)⁺509.1. ¹H NMR (Methanol-d₄, 400 MHz): δ 9.26-9.55 (m, 1H), 8.31 (s, 1H), 7.92-8.24 (m, 6H), 7.76 (d, J=7.03 Hz, 2H), 7.66 (s, 1H), 7.37 (s, 4H), 5.74 (s, 1H), 2.31 (s, 3H), 1.51 (s, 3H).

### Example 20

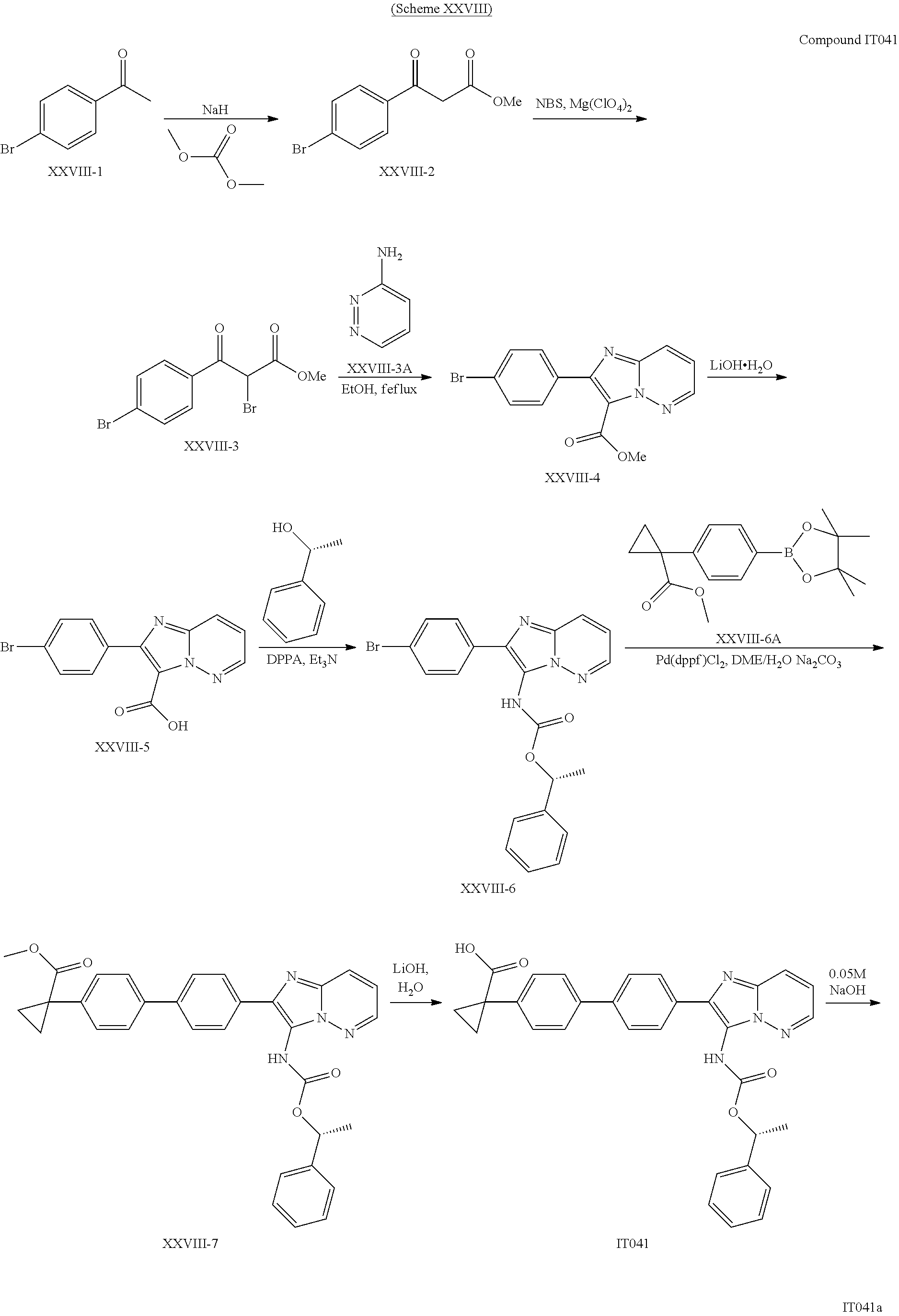

To a solution of XXVIII-1 (19.8 g, 0.1 mol) in THF (200 mL) was added NaH (8 g, 0.2 mol) at 0° C. The mixture was stirred at for 30 min. then added dimethyl carbonate (20 g, 0.3 mol). The solution was stirred at rt for 4 hour. Then NH₄Cl (aq.) was added to quench the solution and the resulting mixture was concentrated, washed and extracted with EA. The combined organic layers were washed with brine, dried over Na₂SO₄ and concentrated under vacuo. The crude was purified by column to afford XXVIII-2 (20.9 g, yield: 80.6%).

To a solution of XXVIII-2 (11.78 g, 45.25 mmol) in MeCN (120 mL) was added NBS (8.86 g, 49.78 mmol) and Mg(ClO₄)₂ (3.08 g, 13.57 mmol) and the resulting mixture was stirred at rt for 1 hour. After the reaction was complete, most of MeCN was removed under reduced pressure. Then 50 mL of H₂O was added and extracted with EA. The combined organic layers were washed with brine, dried over Na₂SO₄ and concentrated under reduced pressure. The crude was purified by column (PE/EA=10/1) to afford XXVIII-3 (9.2 g, yield: 60.33%).

To a solution of XXVIII-3 (4.6 g, 13.65 mmol) in EtOH (40 mL) was added XXVIII-3A (1.36 g, 14.33 mmol). Then the mixture was heated to reflux and stirred at the temperature for 48 hours. After removing most of EtOH under reduced pressure, 30 mL of water was added and extracted with EA. The combined organic layers were washed with brine, dried over Na₂SO₄ and concentrated. The crude was purified by column (PE/EA=10/1) to afford XXVIII-4 (1.26 g, yield: 27.8%).

To a solution of XXVIII-4 (1.26 g, 3.79 mmol) in 10 mL of MeOH/H₂O (v/v=5/1) was added LiOH.H₂O (0.96 g, 22.77 mmol). Then the mixture was heated to 60° C. overnight. MeOH was evaporated and another 10 mL of H₂O was added and extracted with EA. The combined organic layers were washed with brine, dried over Na₂SO₄ and concentrated. The crude product XXVIII-5 (1.1 g, yield: 91.7%) was used to next step directly.

To a solution of XXVIII-5 (900 mg, 2.84 mmol) in toluene (9 mL) was added (R)-1-phenylethanol (416 mg, 3.14 mmol), DPPA (937.8 mg, 3.41 mmol), Et₃N (574 mg, 5.68 mmol) under N₂ atmosphere. Then the mixture was heated to reflux for 2 hours. Then most of toluene was evaporated from the mixture and 10 mL of water was added and extracted with EA. The combined organic layers were washed with brine, dried over Na₂SO₄ and concentrated. The crude product was purified by column (PE/EA=10/1) to afford XXVIII-6 (880 mg, yield: 70%).

XXVIII-7 was prepared by reacting XXVIII-6 and XXVIII-6A following the similar procedure in the synthesis of III-5.

IT041 and IT041a were prepared following the similar procedure in the synthesis of IT001 and IT001a. IT041: MS (ESI) m/z (M+H)⁺519.2. IT041a: ¹H NMR (Methanol-d₄, 400 MHz): δ8.46 (s, 1H), 7.95-8.01 (m, 2H), 7.56-7.66 (m, 4H), 7.40-7.46 (m, 5H), 7.28-7.29 (m, 2H), 5.86-5.87 (d, 1H), 1.65 (br, 3H), 1.50 (s, 2H), 1.05 (s, 2H). MS (ESI) m/z (M+H)⁺519.2.

IT043 was prepared following the similar synthetic scheme for the preparation of IT041 using pyrimidin-2-amine to replace XXVIII-3A. IT043: MS (ESI) m/z (M+H)⁺519.2. Sodium salt IT043a: MS (ESI) m/z (M+H)⁺519.2. ¹H NMR (DMSO-d₆, 400 MHz): δ 8.56 (br, 1H), 8.41 (br, 1H), 7.93 (br, 2H), 7.32-7.63 (m, 11H), 7.04-7.06 (m, 1H), 5.85 (br, 1H), 1.63 (br, 1H), 1.42-1.44 (m, 2H), 0.95-0.96 (m, 2H).

### Example 21

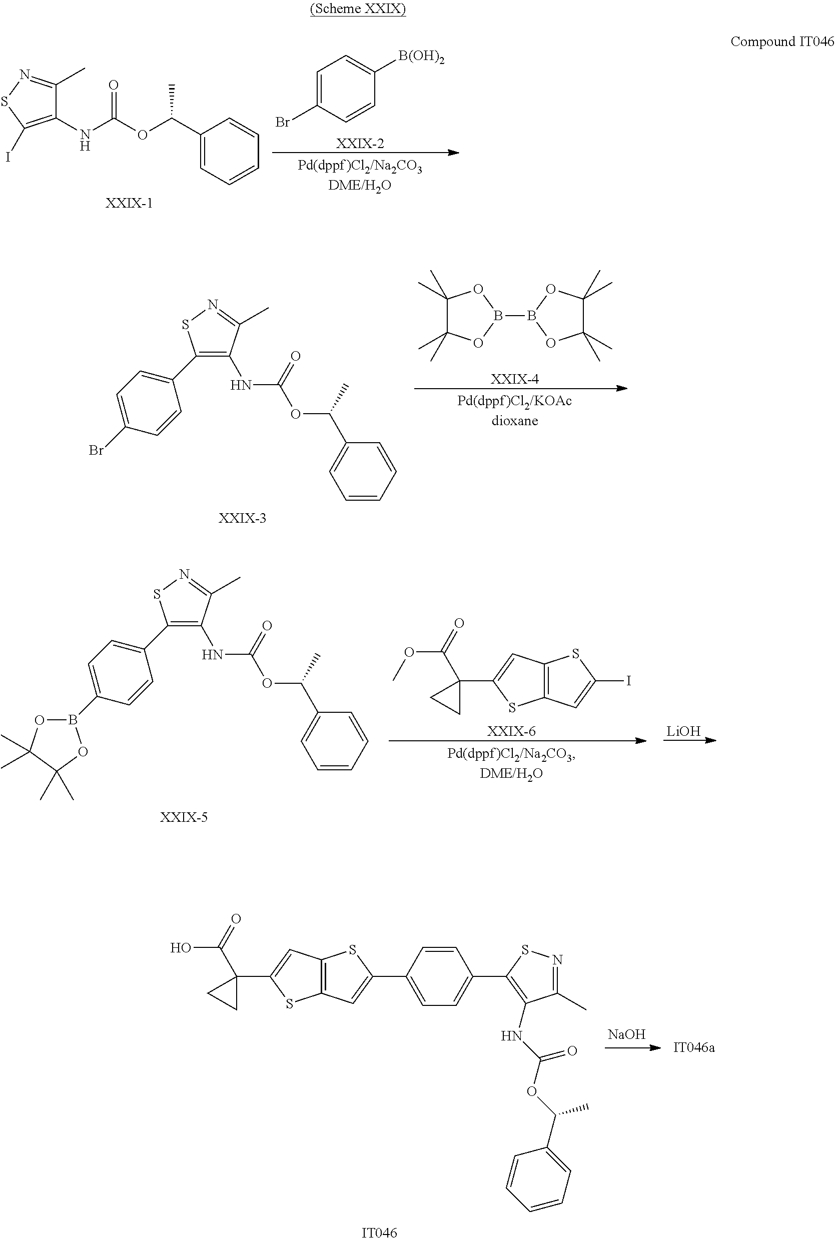

The mixture of XXIX-1 (6.60 g, 17.0 mmol), XXIX-2 (3.62 g, 17.8 mmol), Na₂CO₃ (4.5 g, 42.5 mmol) and Pd(dppf)Cl₂ (124 mg, 0.17 mmol) in DME/H₂O (150 mL, v/v=3/1) was heated to reflux under nitrogen for 12 hours. After concentrated, the residue was partitioned between H₂O and EA, the aqueous phase was extracted with DCM, and the combined organic layer was washed with brine, dried over MgSO₄, concentrated. The residue was purified by column chromatography on silica gel (PE:EA=10:1) to afford XXIX-3 (4.5 g, yield: 63.5%).

The mixture of XXIX-3 (4.5 g, 11 mmol), XXIX-4 (2.93 g, 11.6 mmol), KOAc (2.97 g, 27.5 mmol) and Pd(dppf)Cl₂ (80.4 mg, 0.11 mmol) in dioxane (150 mL, v/v=3/1) was heated to reflux under nitrogen for 12 hours. After concentrated, the residue was partitioned between H₂O and EA, the aqueous phase was extracted with EA, and the combined organic layer was washed with brine, dried over MgSO₄, concentrated. The residue was purified by column chromatography on silica gel (PE:EA=10:1) to afford XXIX-5 (3.8 g, yield: 76.1%).

IT046 was prepared by reacting XXIX-5 and XXIX-6 following the similar procedure for the preparation of XXIX-3 followed by LiOH hydrolysis. Sodium salt IT046a: ¹H NMR (DMSO-d₆, 400 MHz): δ 9.29 (s, 1H), 7.84 (s, 1H), 7.68 (d, J=8.0 Hz, 2H), 7.49 (d, J=8.0 Hz, 2H), 7.31-7.39 (m, 5H), 6.91 (s, 1H), 5.71-5.72 (m, 1H), 2.23 (s, 3H), 1.51 (d, J=6.4 Hz, 3H), 1.45 (br, 2H), 1.01 (br, 2H). MS (ESI) m/z (M+H)⁺561.0.

IT050 was prepared following the synthetic scheme of IT046 using the corresponding carbamate (R)-1-phenylethyl (4-(4-bromophenyl)-1-methyl-1H-1,2,3-triazol-5-yl)carbamate in place of XXIX-3. IT050: MS (ESI) m/z (M+H)⁺545.0. Sodium salt IT050a: ¹HNMR (DMSO-d₆, 400 MHz): δ 10.04 (br, 1H), 7.77 (s, 1H), 7.74 (d, J=8.4 Hz, 2H), 7.66 (d, J=8.4 Hz, 2H), 7.36 (m, 5H), 6.88 (s, 1H), 5.77 (q, J=6.4 Hz, 1H), 3.84 (s, 3H), 1.53 (d, J=6.4 Hz, 3H), 1.44-1.45 (m, 2H), 0.97-0.98 (m, 2H). MS (ESI) m/z (M+H)⁺545.1.

IT051 was prepared following the synthetic scheme of IT046 using the corresponding carbamate (R)-1-phenylethyl (1-(4-bromophenyl)-4-methyl-1H-1,2,3-triazol-5-yl)carbamate in place of XXIX-3. Sodium salt IT051a: ¹H NMR (400 MHz, DMSO-d₆): δ7.83 (s, 1H), 7.75 (d, J=8.4 Hz, 2H), 7.62 (d, J=8.8 Hz, 2H), 7.25-7.34 (m, 5H), 6.91 (s, 1H), 5.69-5.64 (q, 1H), 2.09 (s, 3H), 1.47-1.48 (m, 2H), 1.40 (d, J=6.0 Hz, 3H), 1.00-1.01 (m, 2H). MS (ESI) m/z (M+H)⁺545.1.

IT056 was prepared following a modified synthetic scheme of IT046 by reacting the corresponding (R)-1-phenylethyl (4-(4-aminophenyl)-1-methyl-1H-pyrazol-5-yl)carbamate in place of XXIX-3 in the presence of benzoyl peroxide (BPO), tert-butyl nitrite and acetonitrile. IT056: MS (ESI) m/z (M+H)⁺544.0. Sodium salt IT056a: ¹H NMR (Methanol-d₄, 400 MHz): δ7.74 (s, 1H), 7.52-7.56 (m, 3H), 7.33-7.42 (m, 6H), 7.09-7.16 (m, 2H), 5.84 (d, J=5.6 Hz, 1H), 3.71 (s, 3H), 1.59-1.62 (m, 5H), 1.21-1.23 (m, 2H). MS (ESI) m/z (M+H)⁺544.1.

IT067 was prepared following a modified synthetic scheme of IT046 using (R)-1-phenylethyl (1-(4-bromo-2,5-difluorophenyl)-4-methyl-1H-1,2,3-triazol-5-yl)carbamate (XX-5) in place of XXIX-3. The preparation of XX-5 was described in the synthesis of IT030. IT067: MS (ESI) m/z (M+H)⁺581.0. Sodium salt IT067a: ¹H NMR (DMSO-d₆, 400 MHz): δ 7.85 (s, 1H), 7.71 (br, 1H), 7.37-7.38 (m, 1H), 7.24-7.25 (m, 4H), 7.19-7.21 (m, 1H), 5.67-5.72 (q, 1H), 2.28 (s, 3H), 1.64-1.68 (m, 2H), 1.49 (br, 3H), 1.25-1.28 (m, 2H). MS (ESI) m/z (M+H)⁺581.0.

IT071 was prepared following the synthetic scheme of IT046 using the corresponding (R)-1-phenylethyl (4-(4-bromo-2,5-difluorophenyl)-1-methyl-1H-1,2,3-triazol-5-yl)carbamate in place of XXIX-3. IT071: ¹H NMR (Methanol-d₄, 400 MHz): δ 7.77 (s, 1H), 7.25-7.48 (m, 8H), 5.78 (s, 1H), 3.94 (s, 3H), 1.74-1.75 (m, 2H), 1.59 (s, 3H), 1.45-1.46 (m, 2H). MS (ESI) m/z (M+H)⁺580.9. IT071a: ¹H NMR (DMSO-d₆, T=80, 400 MHz): δ7.89 (s, 1H), 7.54-7.61 (m, 1H), 7.51-7.52 (m, 1H), 7.29-7.35 (m, 5H), 7.22 (s, 1H), 5.77 (q, J=6.4 Hz, 1H), 3.91 (s, 3H), 1.61-1.64 (m, 2H), 1.51 (d, J=6.4 Hz, 3H), 1.26-1.27 (m, 2H). MS (ESI) m/z (M+H)⁺581.0.

### Example 22

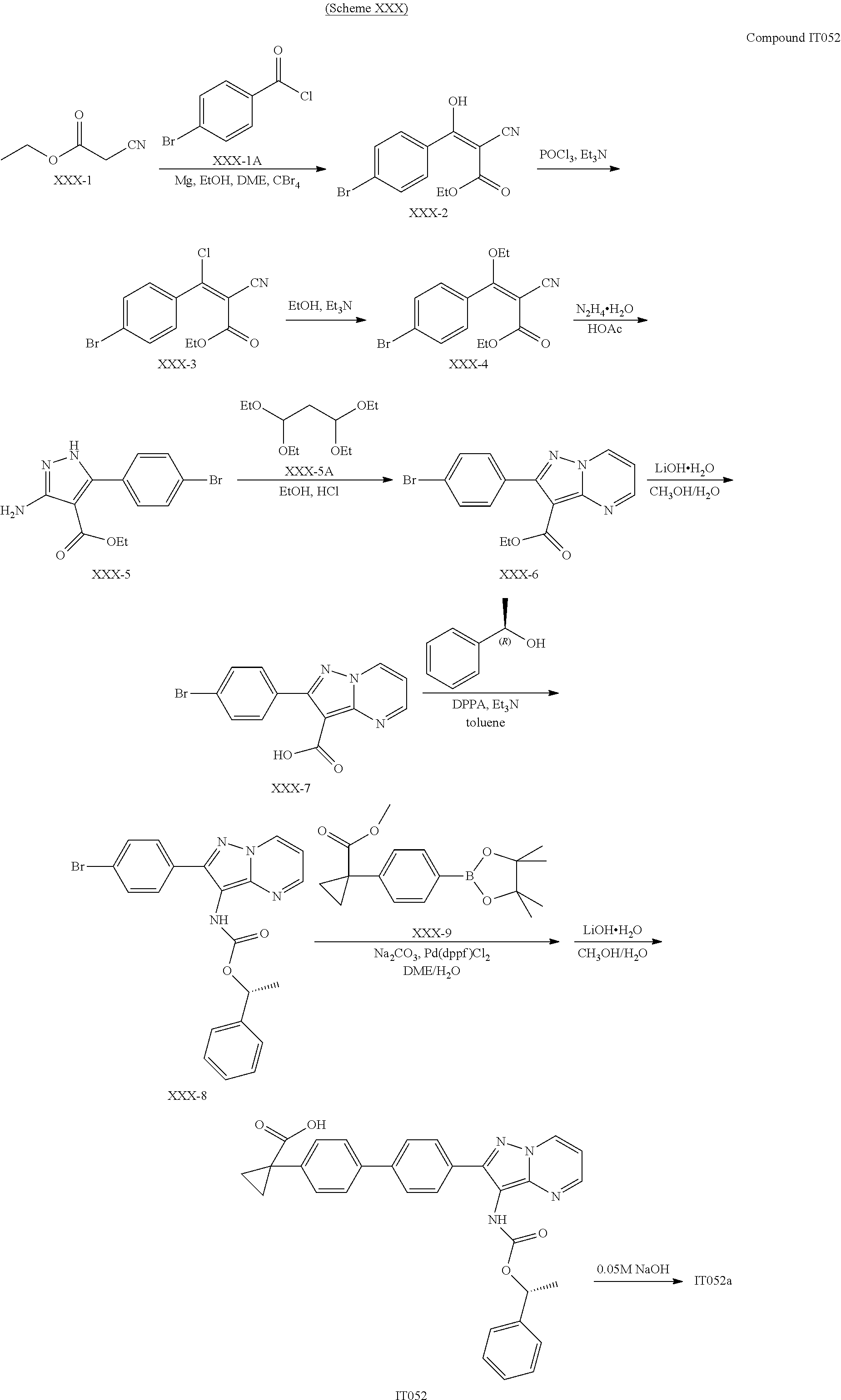

To a stirred solution of Mg (2.1 g, 0.09 mol) in dry EtOH (50 mL) and DME (50 mL) was added CBr₄ (176.1 mg, 0.53 mol). The mixture was heated to 90° C. for overnight. After being cooled to rt, the mixture was evaporated. The magnesium ethoxide formed was dissolved in DME (50 mL) and XXX-1 (10 g, 0.09 mol) was added at 20° C. The solution was cooled to 0° C. and p-bromobenzoyl chloride (19.4 g, 0.09 mol) was added below 40° C. The solution was stirred for 15 hs at rt. The solvent was evaporated and aq. HCl (5 M, 30 mL) was added. The mixture was extracted with DCM. The combined organic layers were washed with water, dried, and concentrated under vacuo. The residue was purified by column chromatography on silica gel (PE:EA=1:1) to give XXX-2 (16 g, yield: 61%).

To a stirred solution of XXX-2 (32.5 g, 0.11 mol), POCl₃ (290.7 mg, 0.72 mmol) in DCM (100 mL) was added dropwise Et₃N (37 g, 0.24 mol). Then the solution was heated to reflux for 15 hs. The solution was extracted with aq.HCl (5 M, 100 mL). The solvent was evaporated and the reminder was dissolved in EtOAc and washed with aq.HCl (5 M) and sodium bicarbonate solution. The organic layers were dried and concentrated under vacuo. The residue was purified by column chromatography on silica gel (PE:EA=10:1) to afford XXX-3 (20 g, yield: 58%).

To a stirred solution of XXX-3 (5 g, 0.016 mol) in EtOH (50 mL) was added Et₃N (8.03 g, 0.08 mol). The mixture was heated to 50° C. for 4 hs. EtOH was removed in vacuo and the residue was purified by column chromatography on silica gel (PE:EA=5:1) to afford XXX-4 (1.7 g, yield: 33%).

To a stirred solution of XXX-4 (1.7 g, 5.25 mmol) in HOAc (50 mL) was added N₂H₄H₂O (0.5 g, 10.5 mmol). The solution was heated to reflux for 1.5 hours. EtOH was removed in vacuo. Brine was added to the residue and extracted with DCM. The combined organic layers were dried and concentrated under vacuo. The residue was purified by column chromatography on silica gel (PE:EA=1:1) to give XXX-5 (680 mg, yield: 42.5%).

To a solution of XXX-5 (680 mg, 2.19 mmol) in 30 mL MeOH was treated with 1,1,3,3-tetraethoxypropane (723.9 mg, 3.29 mmol) and 1 mL HCl. The solution was heated to 60-80° C. for 3 hs. The solvent was removed in vacuo and the residue was purified by column chromatography on silica gel (PE:EA=1:1) to give XXX-6 (314 mg, yield: 41.3%).

XXX-7, XXX-8, IT052 and IT052a were prepared following the similar procedure described in the synthesis of XXII-4, XXII-5, IT033 and IT033a. IT052: MS (ESI) m/z (M+H)⁺519.2. IT052a: ¹HNMR (Methanol-d₄, 400 MHz): δ 8.88-8.89 (br, 1H), 8.53 (br, 1H), 7.94 (d, J=8.0 Hz, 2H), 7.60-7.65 (m, 4H), 7.47-7.48 (m, 4H), 7.27-7.41 (m, 3H), 7.04 (br, 2H), 5.84 (br, 1H), 1.62 (br, 5H), 1.25 (br, 2H). MS (ESI) m/z (M+H)⁺519.2.

### Example 23

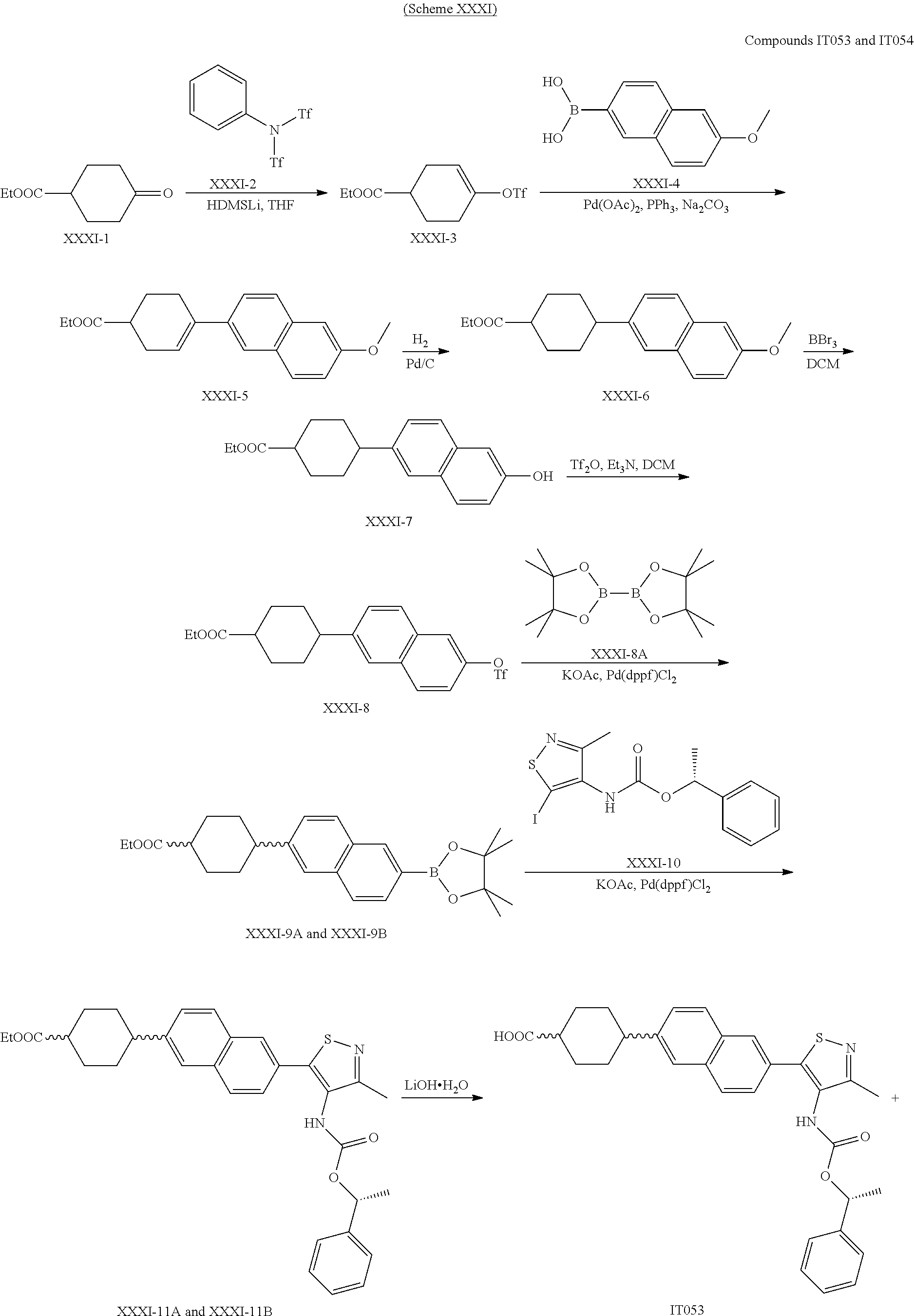

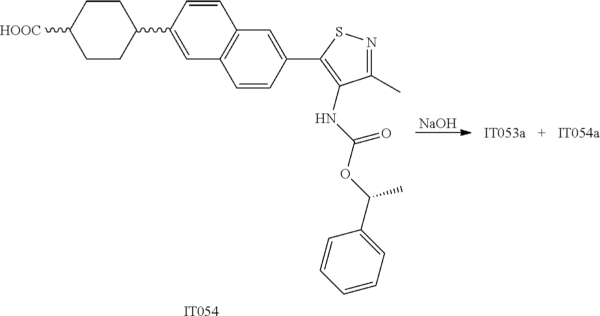

To a solution of XXXI-1 (5 g, 29.4 mmol) in THF (50 mL) was added LiHMDS (30.9 mL, 30.9 mmol) at −78° C. The solution was stirred at −78° C. for 1 h, then XXXI-2 (11 g, 30.9 mmol) in THF (50 mL) was added. The cooling bath was removed after stirring for 30 mins, the solution was stirred at rt overnight. The reaction was quenched with 1N NaHSO₃ and the solvent was evaporated. The residue was partioned between EA and water. The organic layer was washed with 0.5 N NaOH, NH₄Cl and brine, dried over Na₂SO₄ and concentrated to afford XXXI-3 (10 g, crude yield: 100%).

To a stirred solution of XXXI-3 (10 g, 33.1 mmol), XXXI-4 (6.69 g, 33.1 mmol), Na₂CO₃ (7.02 g, 66.2 mmol) and PPh₃ (0.74 g, 3.31 mmol) in EtOH/toluene (120 mL, V/V=1/3) was added Pd(OAc)₂ (0.87 g, 3.31 mmol) under N₂. The mixture was purged with nitrogen for 5 minutes and heated to reflux for 2 hs. After being cooled to rt, the mixture was diluted with water and extracted with EtOAc. The combined organic layers were washed with brine, and concentrated under vacuo. The residue was purified by column chromatography on silica gel (PE:EA=10:1) to give XXXI-5 (5 g, yield: 50%).

To a stirred solution of XXXI-5 (4 g, 12.9 mmol) in MeOH (80 mL) was added Pd/C (2 g, 50%). Then the suspension was degassed under vacuum and purged with H₂ (50 Psi) at rt for 3 hs. Then the solution was filtered and evaporated in vacuo to give XXXI-6 (3.5 g, yield: 88%).

To a stirred solution of XXXI-6 (1 g, 3.2 mmol) in DCM (10 mL) was added BBr₃ (3.1 g, 12.8 mmol) dropwise at −78° C. Then it was stirred at rt for 4 hs. The mixture was quenched with H₂O. The organic layers were washed with brine, and concentrated under reduced pressure to give XXXI-7 (0.94 g, yield: 100%).

To a stirred solution of XXXI-7 (0.94 g, 3.15 mmol) and Et₃N (0.96 g, 9.46 mmol) in DCM (10 mL) was added Tf₂O (1.08 g, 3.8 mmol) under nitrogen at 0° C. and the mixture was stirred overnight. 10 mL of H₂O was added and the aqueous phase was extracted with DCM. The organic layer was combined and washed with brine, dried over Na₂SO₄, concentrated in vacuo to afford XXXI-8 (1.35 g, crude yield: 100%).

XXXI-9A, XXXI-9B, XXXI-11A and XXXI-11B were prepared following the similar procedure described in the synthesis of III-3.

IT053, IT054 and their sodium salts IT053a, IT054a were prepared following the similar procedure described in the synthesis of IT001 and IT001a. IT053 and IT054: MS (ESI) m/z (M+H)⁺515.2.

IT053a: MS (ESI) m/z (M+H)⁺515.1. ¹HNMR (DMSO-d₆, 400 MHz) δ 9.35 (br, 1H), 8.02 (s, 1H), 7.91 (d, J=8.4 Hz, 1H), 7.79 (d, J=8.0 Hz, 1H), 7.69 (s, 1H), 7.55 (d, J=8.4 Hz, 1H), 7.24-7.47 (m, 5H), 7.09 (br, 1H), 5.72 (br, 1H), 2.61-2.69 (m, 1H), 2.16-2.29 (m, 6H), 1.75-1.82 (m, 2H), 1.61-1.64 (m, 2H), 1.44-1.52 (m, 5H).

IT054a: MS (ESI) m/z (M+H)⁺515.1. ¹HNMR (Methanol-d₄, 400 MHz) δ 7.94 (s, 1H), 7.82 (d, J=7.6 Hz, 1H), 7.68-7.70 (m, 2H), 7.42-7.49 (m, 2H), 7.25-7.32 (m, 4H), 7.00 (s, 1H), 5.75 (br, 1H), 2.71 (s, 1H), 2.23-2.32 (m, 4H), 1.99-2.25 (m, 4H), 1.52-1.66 (m, 7H).

### Example 24

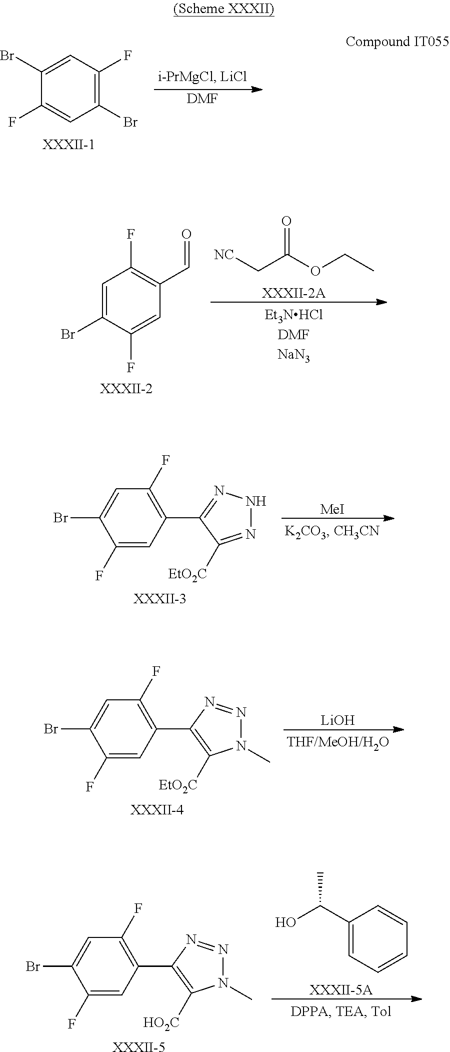

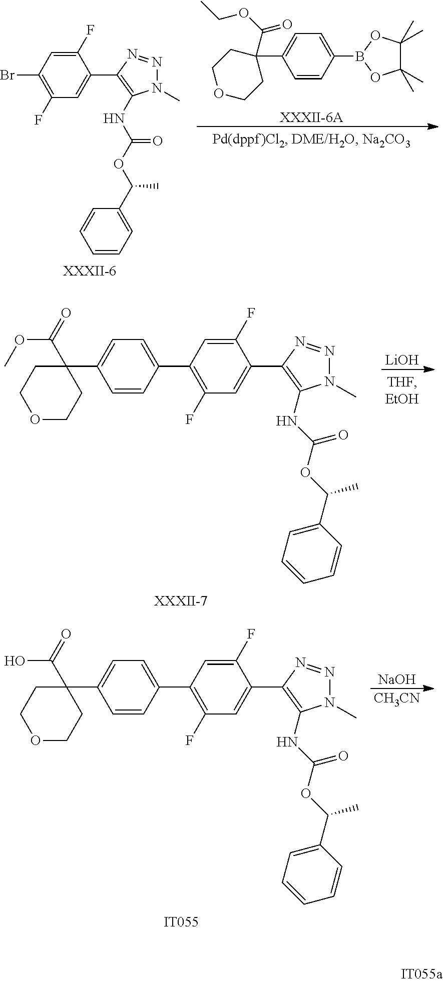

To a stirred solution of XXXII-1 (12 g, 44.1 mmol) in THF (150 mL) was added dropwise of XXXII-1A (44.1 mmol, 34 mL, 1.3 M) at −40° C. After stirred 1 h at −40° C., DMF (64 g, 882 mmol) was added and the mixture was stirred overnight. NH₄Cl (aq., 2M) was added and the mixture was extracted with EtOAc. The organic phase was dried with Na₂SO₄. The solvent was removed in vacuo and the residue was purified by column chromatography (PE/EA=10/1) to afford XXXII-2 (6.5 g, yield: 66.7%).

To a solution of XXXII-2 (3 g, 13.6 mmol) in DMF (30 mL), Et₃N.HCl (4.66 g, 34 mmol) were added NaN₃ (2.4 g, 40.8 mmol) and XXXII-2A (1.53 g, 13.6 mmol). The reaction mixture was heated at 70° C. and stirred overnight under nitrogen protection. After completion of the reaction, the mixture was poured into water and extracted with EtOAc. The organic phase was dried with Na₂SO₄. The solvent was removed in vacuo and the residue was purified by column chromatography (PE:EA=3:1) to afford XXXII-3 (0.5 g, yield: 11%).

XXXII-4, XXXII-5 and XXXII-6 were prepared following the similar procedure described in the synthesis of XXIV-3, XXIV-4 and XXIV-5.

XXXII-7, IT055, and IT055a were prepared following the similar procedure described in the synthesis of III-5, IT001 and IT001a. IT055: MS (ESI) m/z (M+H)⁺562.5. IT055a: ¹HNMR (DMSO-d₆ 400 MHz) δ7.55-7.49 (m, 5H), 7.35-7.28 (m, 6H), 5.78 (q, 1H), 3.90 (s, 3H), 3.82-3.78 (m, 2H), 3.60-3.55 (m, 2H), 2.51-2.43 (m, 2H), 1.87-1.80 (m, 2H), 1.49 (d, J=6.0 Hz, 3H). MS (ESI) m/z (M+H)⁺563.1.

### Example 25

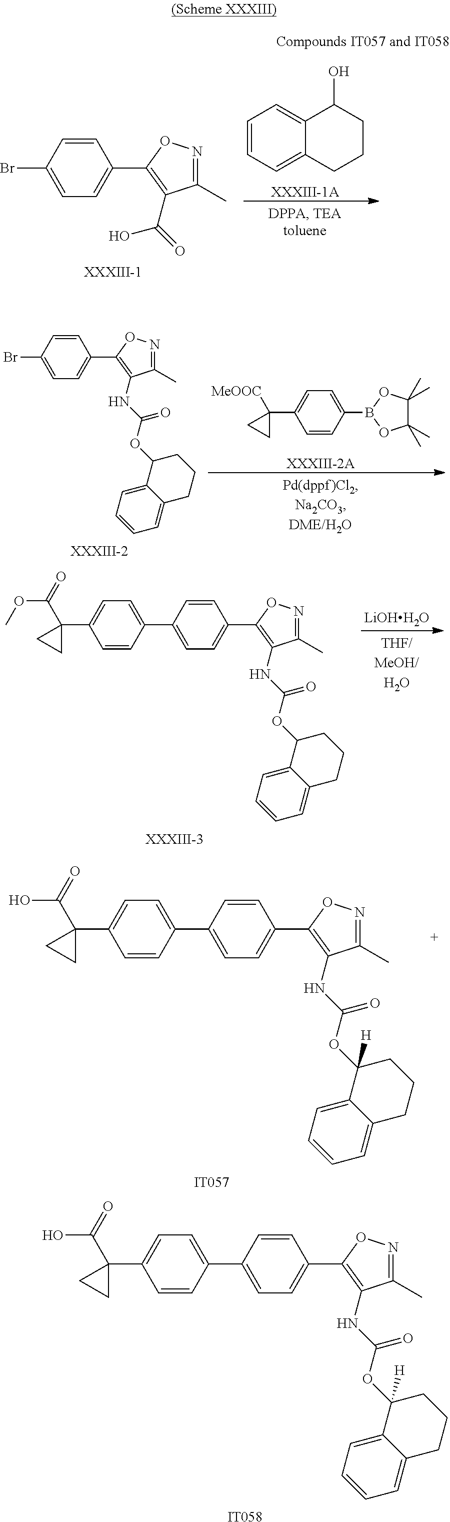

To a solution of XXXIII-1 (1 g, 3.6 mmol) in dry toluene (10 mL) was added XXXIII-1A (0.639 g, 4.3 mmol), TEA (0.763 g, 7.2 mmol) and DPPA (1.18 g, 4.3 mmol). The reaction mixture was heated to 80° C. for 6 h. The mixture was diluted with EtOAc, washed with brine, dried over Na₂SO₄, filtered and concentrated. The residue was purified by column chromatography (PE:EA=5:1) to give XXXIII-2 (1.3 g, yield 84.9%).

XXXIII-3 prepared by reacting XXXIII-2 with XXXIII-2A following the similar procedure described in the synthesis of III-5,

IT057 and IT058 were prepared following the similar procedure described in the synthesis of IT001, followed by chiral separation by SFC. MS (ESI) m/z (M+H)⁺ 509.1.

Sodium salt IT057a: ¹HNMR (400 MHz, DMSO-d₆) δ9.25 (s, 1H), 7.80-7.87 (m, 4H), 7.57 (d, J=8.0 Hz, 2H), 7.36 (d, J=8.0 Hz, 2H), 7.05-7.24 (m, 4H), 5.85 (br, 1H), 2.77-2.89 (m, 2H), 2.21 (s, 3H), 1.85-2.09 (m, 4H), 1.22 (br, 2H), 0.73 (br, 2H). MS (ESI) m/z (M+H)⁺509.2.

Sodium salt IT058a: ¹H NMR (400 MHz, DMSO-d₆) δ 9.26 (s, 1H), 7.79-7.87 (m, 4H), 7.56 (d, J=8.0 Hz, 2H), 7.25 (d, J=8.0 Hz, 2H), 7.06-7.19 (m, 4H), 5.85 (s, 1H), 2.76-2.88 (m, 3H), 2.21 (s, 3H), 1.85-2.03 (m, 4H), 1.22 (br, 2H), 0.73 (br, 2H). MS (ESI) m/z (M+H)⁺509.2.

### Example 26

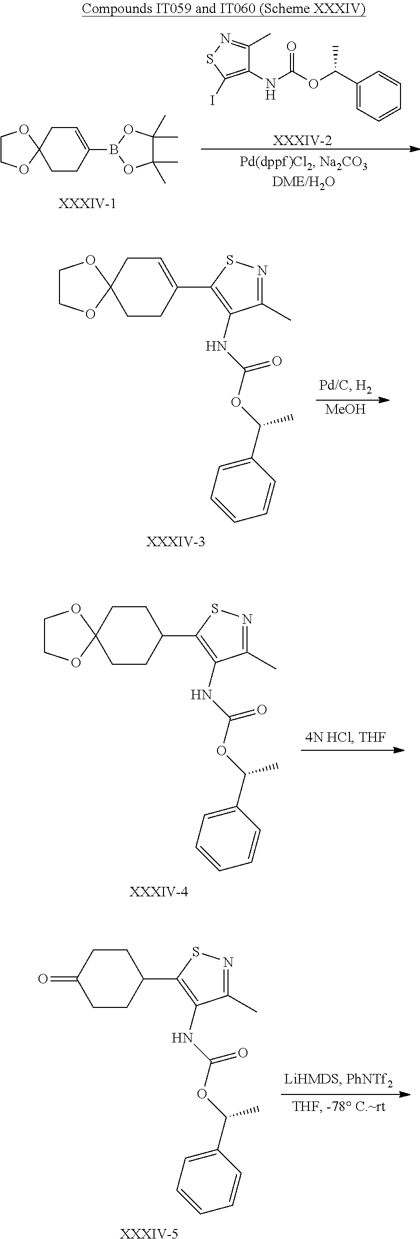

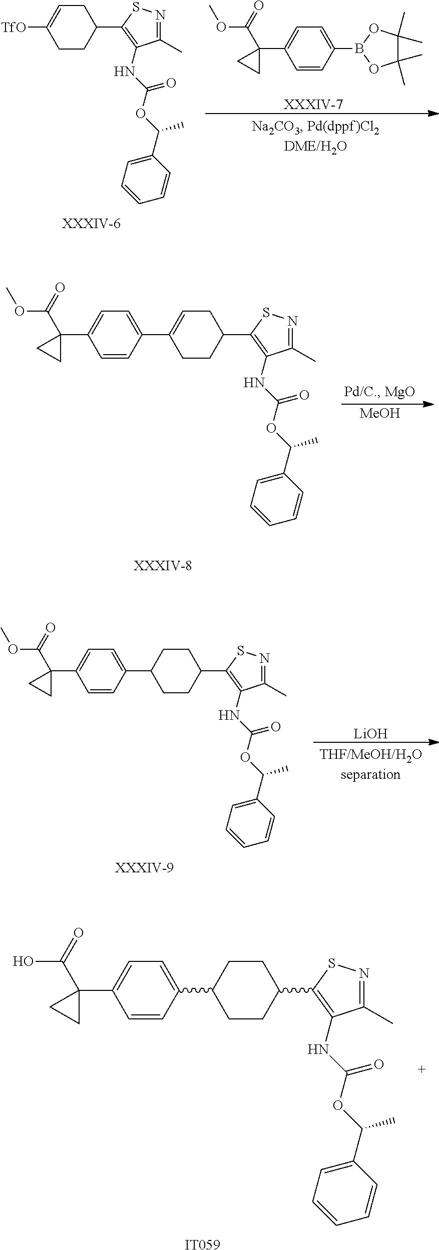

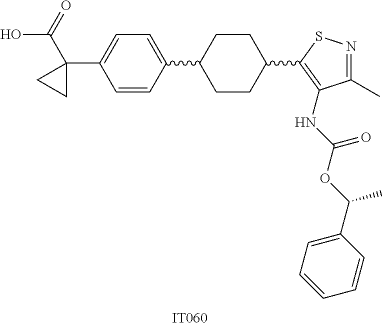 [SMILES: Cc1nsc(C2CCC(c3ccc(C4(C(=O)O)CC4)cc3)CC2)c1NC(=O)O[C@H](C)c1ccccc1 ; "IT060"]

Argon gas was bubbled through a mixture of XXXIV-1 (2.0 g, 7.52 mmol) and XXXIV-2 (2.92 g, 7.52 mmol) in 30 mL of DME/H₂O (v/v=3/1). The Na₂CO₃ (2.39 g, 22.56 mmol) and Pd(dppf)Cl₂ (275 mg, 0.38 mmol) was added. The mixture was heated to 80° C. and stirred overnight. After cooled, the mixture was filtered through Celite and the filtrate was washed with brine, dried over MgSO₄ and concentrated. The residue was purified by flash column chromatography over silica gel (PE:EA=2/1) to afford XXXIV-3 (2.3 g, yield 77%).

A mixture of XXXIV-3 (2 g, 4.99 mmol) and 3 g of Pd/C (w %=10%) in 100 mL of methanol was hydrogenated under hydrogen atmosphere (40 psi) for 20 hours at rt. The mixture was filtered through Celite and the filtrate was concentrated in vacuum to afford XXXIV-4 (1.7 g, yield 85%).

4N aqueous HCl solution (17 mL, 68 mmol) was added slowly to a solution of XXXIV-4 (1.7 g, 4.23 mmol) in 34 mL of THF at 0° C. The mixture was stirred for 5 hs at rt. The mixture was diluted with H₂O, extracted with EA. The combined organic layer was washed with saturated NaHCO₃ solution, brine, dried over MgSO₄ and concentrated. The residue was purified by flash column chromatography over silica gel (PE:EA=5/1) to afford XXXIV-5 (1.2 g, yield 80%).

To a stirred solution of XXXIV-5 (800 mg, 2.23 mmoL) in dry THF (10 mL) was added LiHMDS (1.0N solution in THF, 11.2 mmol) dropwise at −78° C. After addition, the reaction temperature was allowed to rise to rt slowly and the mixture was stirred for 1 h at rt. Then the mixture was re-cooled to −78° C. and a solution of PhNTf₂ (1.6 g, 4.46 mmol) in 2 mL of THF was added slowly. After addition, the reaction temperature was allowed to rise to rt slowly and the mixture was stirred overnight at rt. The reaction mixture was quenched with saturated NH₄Cl aqueous solution, extracted with EtOAc. The combined organic layer was washed with brine, dried and concentrated. The residue was purified by flash column chromatography over silica gel (PE:EA=7/1) to afford XXXIV-6 (300 mg, yield 27.3%).

XXXIV-8 was prepared by reacting XXXIV-6 (120 mg, 0.24 mmol) with XXXIV-7 (148 mg, 0.49 mmol) using the same reaction for the preparation of XXXIV-3 as colourless oil.

A mixture of XXXIV-8 (140 mg, 0.27 mmol), MgO (22 mg, 0.54 mmol) and 210 mg of Pd/C (w %=10%) in 10 mL of MeOH was stirred for 5 h under hydrogen atmosphere at rt. The insoluble substance was filtered off and the filtrate was concentrated in vacuum to afford XXXIV-9 (115 mg, yield 82%) as white solid.

IT059 and IT060 were obtained from LiOH hydrolysis of XXXIV-9 followed by separation. Sodium salt IT059a: ¹H NMR (Methanol-d₄, 400 MHz) δ 7.45-7.46 (m, 2H), 7.39-7.41 (m, 2H), 7.29-7.33 (m, 3H), 7.12 (d, J=8.0 Hz, 2H), 5.82 (q, J=6.4 Hz, 1H), 2.88-2.91 (m, 1H), 2.53-2.55 (m, 1H), 2.27 (s, 3H), 2.05-2.11 (m, 2H), 1.88-1.91 (m, 2H), 1.49-1.62 (m, 7H), 1.41-1.42 (m, 2H), 0.92-0.93 (m, 2H). MS (ESI) m/z (M+H)⁺505.2.

Sodium salt IT060: ¹H NMR (Methanol-d₄, 400 MHz): δ 7.37-7.43 (m, 4H), 7.28-7.30 (m, 3H), 7.16 (d, J=8.0 Hz, 2H), 5.80 (q, J=6.4 Hz, 1H), 3.41 (br, 1H), 2.68 (br, 1H), 2.27 (s, 3H), 1.78-1.90 (m, 8H), 1.60 (d, J=6.4 Hz, 3H), 1.46 (br, 2H), 1.00 (br, 2H). MS (ESI) m/z (M+Na)⁺505.02.

### Example 27

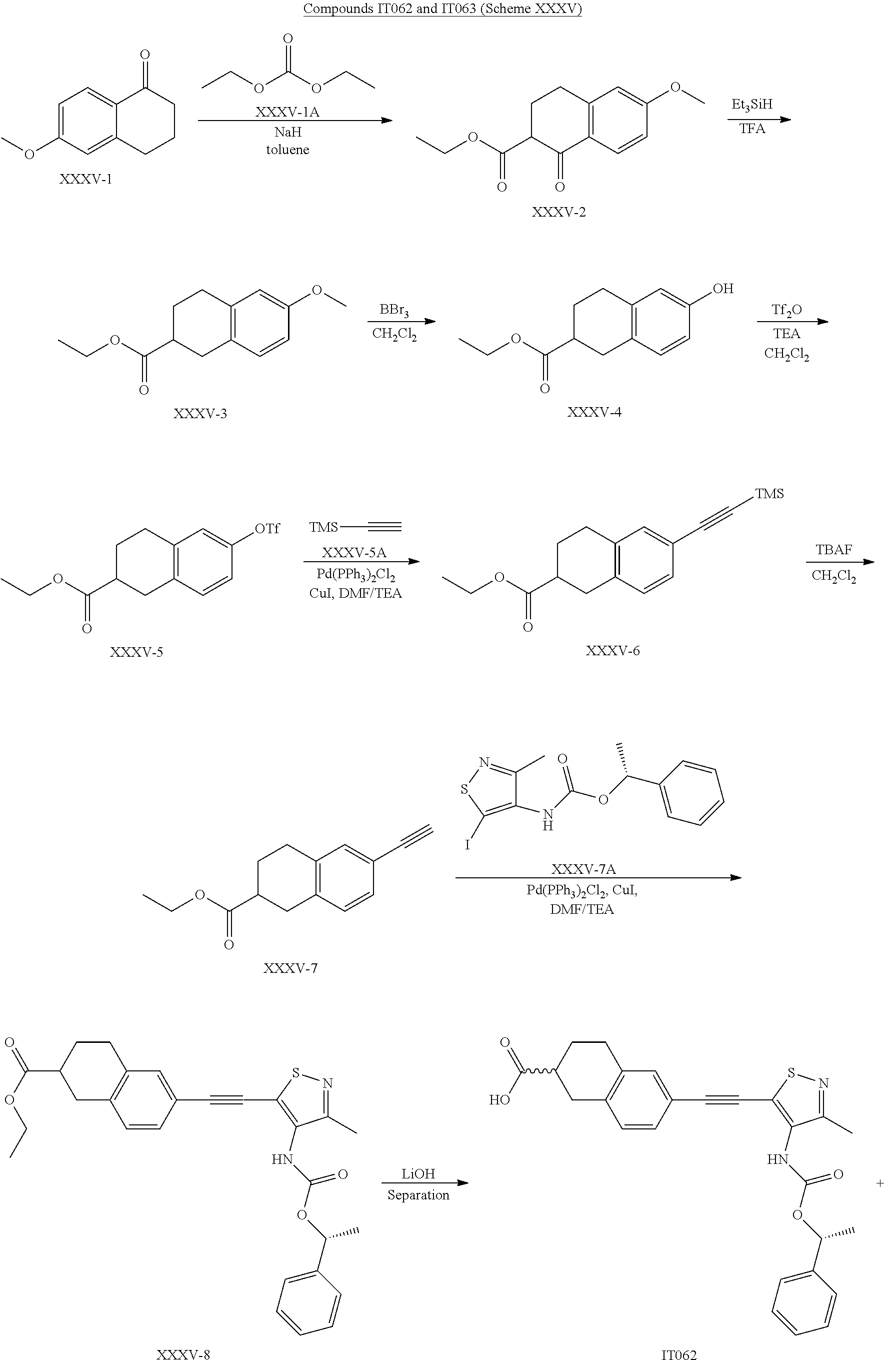

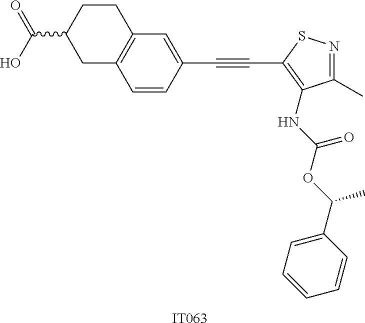 [SMILES: Cc1nsc(C#Cc2ccc3c(c2)CCC(C(=O)O)C3)c1NC(=O)O[C@H](C)c1ccccc1 ; "IT063"]

To a solution of XXXV-1A (7.4 g, 62.7 mmol) in toluene (100 mL) was added portion wise NaH (3.7 g, 92.5 mmol) at 25° C. and the mixture was heated at 120° C. for 30 min. Then to the mixture was added a solution of XXXV-1 (5.1 g, 18.5 mmol) in toluene (50 mL). The resulting mixture was stirred at 120° C. for 12 h. After being cooled to rt, aq. HCl (1M, 20 mL) was added to the mixture, and the mixture was extracted with EtOAc. The organics were combined, dried with Na₂SO₄, and concentrated to afford crude XXXV-2 (5.0 g, yield: 78.1%), which was used to next step directly.

To a solution of XXXV-2 (5 g, 20.16 mmol) in TFA (50 mL) was added Et₃SiH (9.5 mL) dropwise, and the resulting mixture was stirred at 25° C. for 12 h. Removed the solvent in vacuo gave an oily residue, which was washed with H₂O, extracted with EtOAc, washed with saturated NaHCO₃. The organics were combined, dried with Na₂SO₄, filtered and concentrated. The residue was purified by column chromatography (PE/EA=30/1) to give XXXV-3 (4 g, yield: 85%).

To a solution of XXXV-3 (1.5 g, 6.4 mmol) in CH₂Cl₂ (30 mL) was added BBr₃ (3.2 g, 12.8 mmol) at −68° C. dropwise. After addition, the mixture was stirred at 25° C. for 2 h. The reaction was poured into ice-water, extracted with CH₂Cl₂. The organic layer was washed with brine, dried over Na₂SO₄, filtered and concentrated. The residue was purified by column chromatography (PE/EA=3/1) to give XXXV-4 (325 mg, yield: 23%).

To a stirred solution of XXXV-4 (625 mg, 2.84 mmol) and TEA (573 mg, 5.68 mmol) in CH₂Cl₂ (20 mL) was added Tf₂O (941 mg, 3.4 mmol) dropwise at −40° C. The mixture was stirred at 18° C. for 2 h. Then H₂O (20 mL) was added, the organic layer were separated, dried with Na₂SO₄, and concentrated to afford crude XXXV-5 (960 mg, yield: 96%), which was used to next step directly.

XXXV-6, XXXV-7 and XXXV-8 were prepared following the similar procedure described in the synthesis of XVII-2, XVII-3, and XVII-5.

IT062 and IT063 were obtained from LiOH hydrolysis of XXXV-8 followed by SFC separation. MS (ESI) m/z (M+H)⁺461.1.

Sodium salt IT062a: ¹HNMR (DMSO-d₆, 400 MHz) δ 9.59 (brs, NH), 7.29-7.39 (m, 5H), 7.11-7.15 (m, 3H), 5.77-5.82 (m, 1H), 2.67-2.85 (m, 4H), 2.28 (s, 3H), 2.21-2.23 (m, 1H), 1.98-2.01 (m, 1H), 1.61-1.64 (m, 1H), 1.53 (d, J=6.0 Hz, 3H). MS (ESI) m/z (M+H)⁺461.1.

Sodium salt IT063a: ¹HNMR (DMSO-d₆, 400 MHz) δ9.56 (brs, 1H), 7.31-7.39 (m, 5H), 7.12-7.16 (m, 3H), 5.77-5.82 (q, 1H), 2.65-2.87 (m, 4H), 2.36-2.37 (m, 1H), 2.28 (s, 3H), 2.01-2.04 (m, 1H), 1.64-1.65 (m, 1H), 1.52-1.53 (d, J=6.0 Hz, 3H). MS (ESI) m/z (M+H)⁺461.1.

### Example 28

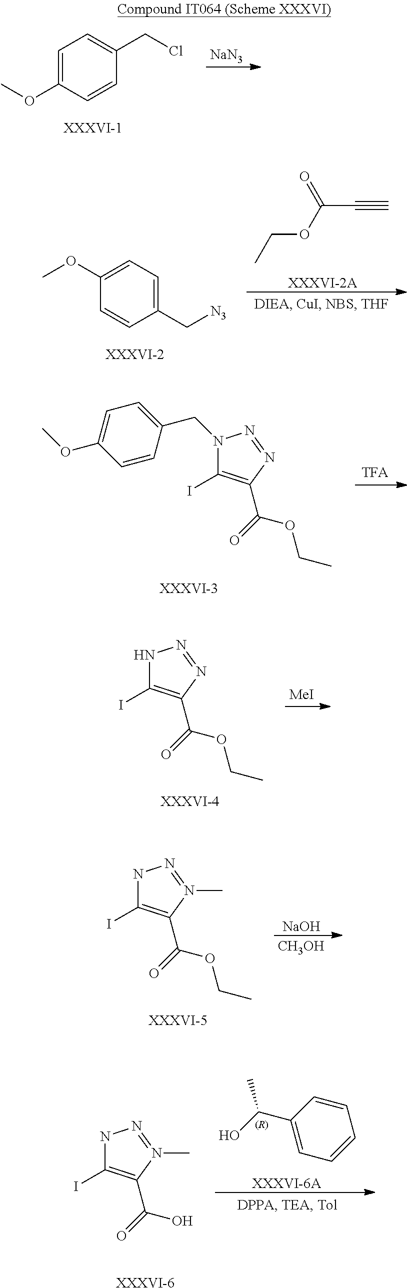

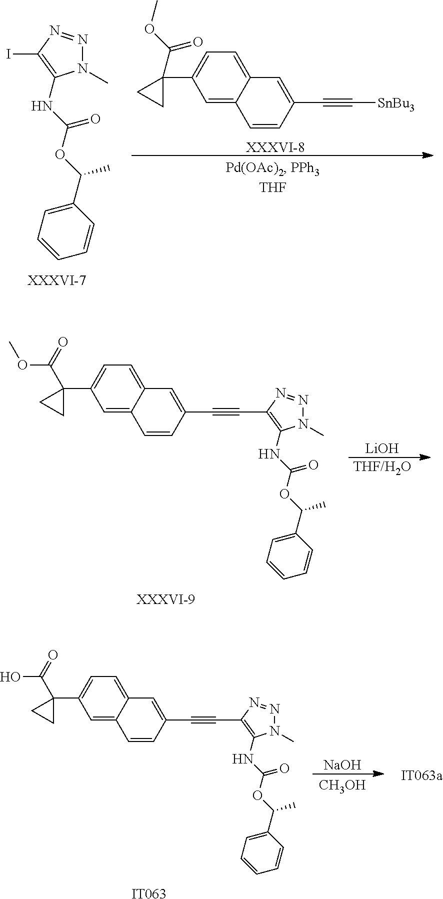

To a solution of XXXVI-1 (20 g, 0.127 mol) in DMF (150 mL) was added NaN₃ (8.2 g, 0.127 mol). After addition, the mixture was stirred for 24 h at 25° C. The reaction mixture was extracted with MTBE. The combined organic phase was washed with brine, dried over Na₂SO₄, filtered and concentrated to give crude XXXVI-2 (20.8 g, crude yield: 100%), which was used to next step directly.

To a solution of XXXVI-2 (20.8 g, 0.127 mol) in THF (200 mL) was added ethyl propiolate XXXVI-2A (12.5 g, 0.127 mol), CuI (24.2 g, 0.127 mol), DIEA (16.4 g, 0.127 mol) and NBS (25 g, 0.25 mol). The reaction mixture was flushed with nitrogen and stirred for 3 h. Water was added and extracted with EtOAc. The organic layer was combined, dried over Na₂SO₄, and concentrated. The residue was purified by column chromatography (PE:EA=5:1) to give XXXVI-3 (20 g, yield: 40.8%).

A mixture of XXXVI-3 (20 g, 51.7 mmol) in TFA (200 mL) was stirred at 65° C. for 3 h. The reaction mixture was concentrated, and the residue was purified by column chromatography (PE:EA=5:1) to give XXXVI-4 (12 g, yield: 87.6%).

To a solution of XXXVI-4 (12 g, 45 mmol) in CH₃CN (100 mL) was added MeI (12.7 g, 90 mmol), K₂CO₃ (12.4 g, 90 mmol). The reaction mixture was stirred for 3 hs at 25° C. The mixture was diluted with EtOAc, washed with brine, dried over Na₂SO₄, filtered and concentrated. The residue was purified and separated by prep-HPLC to give XXXVI-5 (2.1 g, yield: 13.3%). The structure was confirmed by HMBC.

XXXVI-6 was prepared from XXXVI-5 following the similar procedure described in the synthesis of XII-4 using NaOH in place of LiOH.

XXXVI-7 was prepared from reacting XXXVI-6 with XXXVI-6A following the similar procedure described in the synthesis of XII-5.

XXXVI-9 was prepared from reacting XXXVI-7 with XXXVI-8 following the similar procedure described in the synthesis of XII-8.

IT064 and sodium salt IT064a were prepared following the similar procedure described in the synthesis of IT001 and IT001a. IT064a: ¹H NMR (DMSO-d₆ 400 MHz) δ 7.95 (s, 1H), 7.71-7.82 (m, 3H), 7.68 (s, 1H), −7.92 (m, 3H), 7.42 (d, J=7.6 Hz, 1H), 7.33-7.37 (m, 6H), 5.77-5.81 (q, 1H), 3.78 (s, 3H), 1.51 (d, J=6.4 Hz, 3H), 1.28 (d, J=2.4 Hz, 2H), 0.85 (br, 2H). MS (ESI) m/z (M+H)⁺481.1.

IT070 was prepared following the general synthetic scheme of IT064 replacing XXXVI-8 with

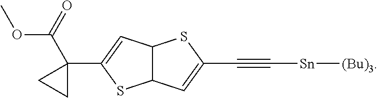

MS (ESI) m/z (M+H)⁺493.0. Sodium salt IT070a: ¹H NMR (DMSO-d₆ 400 MHz): δ 7.7.34-7.40 (m, 2H), 7.25-7.34 (m, 4H), 7.02 (s, 1H), 5.82-5.87 (m, 1H), 3.87 (s, 3H), 1.58-1.59 (m, 5H), 1.17 (br, 2H). MS (ESI) m/z (M+H)⁺493.0.

### Example 29

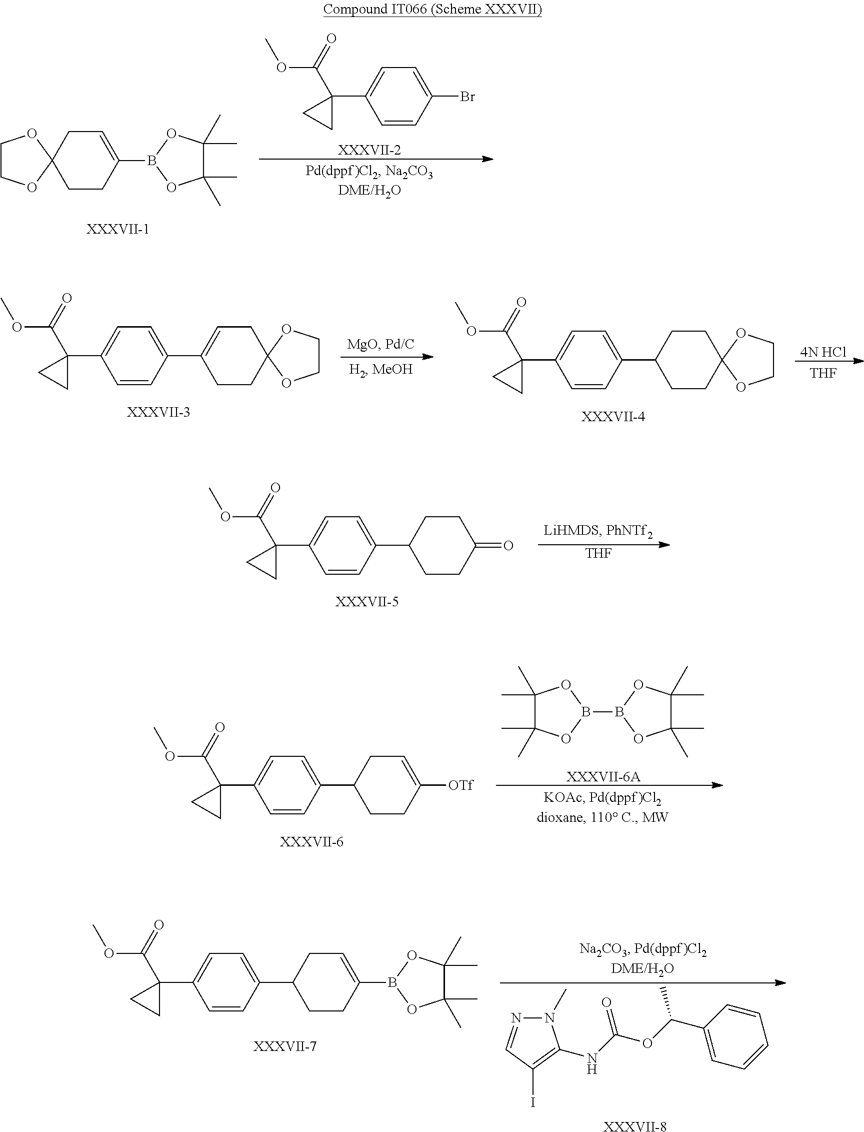

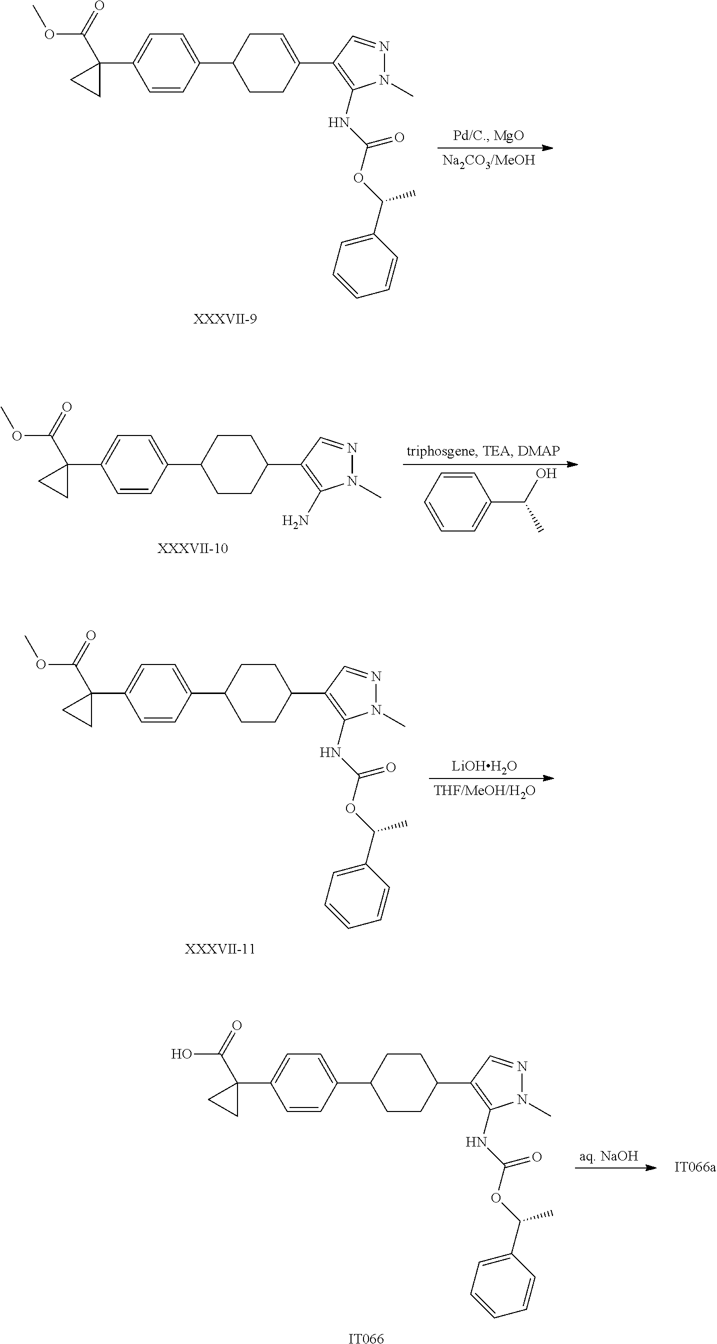

XXXVII-3 was prepared by reacting XXXVII-1 with XXXVII-2 following the similar procedure described in the synthesis of III-5.

A mixture of XXXVII-3 (2.86 g, 9.10 mmol) and 430 mg of Pd/C (w %=5%) in 100 mL of methanol was hydrogenated under hydrogen atmosphere (35 psi) for 20 hours. The mixture was filtered through Celite and the filtrate was concentrated in vacuum to afford XXXVII-4 (2.7 g, yield 94%).

4N aqueous HCl solution (20 mL, 80 mmol) was added slowly to a solution of XXXVII-4 (2.7 g, 8.53 mmol) in 40 mL of THF at 0° C. The mixture was stirred for 2 hs at rt. The mixture was diluted with H₂O, extracted with EtOAc. The combined organic layer was washed with saturated NaHCO₃ solution, brine, dried over MgSO₄ and concentrated. The residue was purified by flash column chromatography over silica gel (PE:EA=5/1) to afford XXXVII-5 (2.3 g, yield 99%).

XXXVII-6 was prepared from XXXVII-5 following the similar procedure described in the synthesis of XXXIV-6. XXXVII-7 was prepared from reacting XXXVII-6 with XXXVII-6A following the similar procedure described in the synthesis of III-3.

XXXVII-9 was prepared following the similar procedure described in the synthesis of III-5.

A mixture of XXXVII-9 (110 mg, 0.22 mmol), MgO (18 mg, 0.44 mmol), Na₂CO₃ (46 mg, 0.44 mmol) and 22 mg of Pd/C (w %=5%) in 10 mL of MeOH was hydrogenated under hydrogen atmosphere (35 psi) at rt. The insoluble substance was filtered off and the filtrate was concentrated. The residue was treated with EtOAc and H₂O. The organic layer was separated and the aqueous layer was extracted with EtOAc. The combined organic layer was washed with brine, dried and concentrated to afford XXXVII-10 (50 mg, yield 64%).

Triphosgene (84 mg, 0.28 mmol) was added to a solution of XXXVII-10 (100 mg, 0.28 mmol), TEA (143 mg, 1.41 mmol) and DMAP (35 mg, 0.28 mmol) in 5 mL of dry dichloromethane at 5° C. Then (R)-1-phenylethanol (172 mg, 1.41 mmol) was added. The mixture was stirred overnight at rt. The mixture was diluted with dichloromethane, washed with H₂O, saturated NaHCO₃ aqueous solution, brine, dried and concentrated to afford XXXVII-11 (150 mg, crude), which was used directly without further purification.

IT066 and sodium salt IT066a were prepared following the similar procedure described in the synthesis of IT001 and IT001a. IT066: MS (ESI) m/z (M+H)⁺488.1. IT066a: ¹HNMR (400 MHz, Methanol-d₄) δ7.25-7.44 (m, 8H), 7.09-7.13 (m, 2H), 5.79-5.87 (m, 1H), 3.64 (s, 3H), 2.63-2.87 (m, 1H), 2.50-2.57 (m, 1H), 1.61-1.92 (m, 6H), 1.49-1.54 (m, 5H), 1.40-1.41 (m, 2H), 0.91-0.93 (m, 2H). MS (ESI) m/z (M+H)⁺488.2.

### Example 30

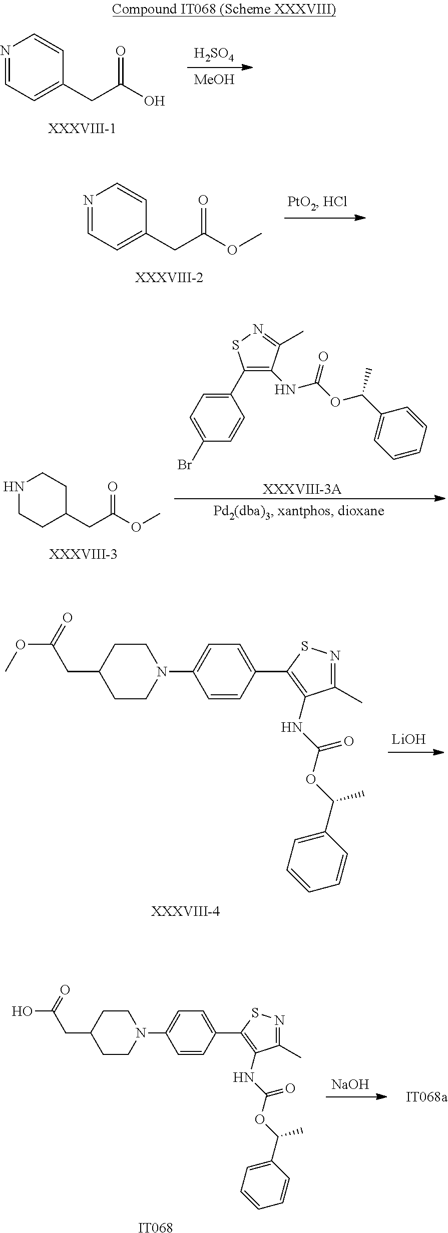

To a solution of XXXVIII-1 (4 g, 29.2 mmol) in MeOH (40 mL) was dropwise H₂SO₄ (1 g). Then the mixture was heated to reflux for about 2 hs. Then the MeOH was evaporated in vacuo. Water was added and extracted with EtOAc. The combined organic layers were washed with brine, dried over Na₂SO₄ and concentrated under vacuo. The crude product was purified by column chromatography (PE:EA=10/1) to afford XXXVIII-2 (3.5 g, yield: 79.55%).

To a solution of XXXVIII-2 (2 g, 13.25 mmol) in MeOH (20 mL) was added PtO₂ (200 mg) and HCl (6N, 2 mL) under H₂ atmosphere (30 Psi) at rt. Then the mixture was stirred at this atmosphere for about 2 hs. Then the solution was filtered and the liquid was concentrated. The crude XXXVIII-3 (1.8 g, yield: 86.5%) was used to next step directly.

To a solution of XXXVIII-3 (136 mg, 0.866 mmol) in dioxane (4 mL) was added compound XXXVIII-3A (300 mg, 0.72 mmol) and Xantphos (117 mg, 0.17 mmol) and Cs₂CO₃ (468 mg, 1.732 mmol) and Pd₂(dba)₃ (119 mg, 0.17 mmol) under N₂ atmosphere. Then the mixture was heated to reflux and stirred for 4 hs. Then dioxane was removed under vacuo, water (2 mL) was added and extracted with EtOAc. The combined organic layers were washed with brine, dried over Na₂SO₄ and concentrated under vacuo. The residue was purified by column chromatography (PE:EA=5/1) to afford XXXVIII-4 (120 mg, yield: 28.98%).

IT068 and sodium salt IT068a were prepared following the similar procedure described in the synthesis of IT001 and IT001a. IT068: MS (ESI) m/z (M+H)⁺480.1. IT068a: ¹H NMR (DMSO-d₆, 400 MHz) δ 8.69 (s, 1H), 7.27-7.32 (m, 7H), 6.86-6.88 (m, 2H), 5.68-5.73 (m, 1H), 3.71-3.73 (m, 2H), 2.73-2.79 (m, 2H), 2.18 (s, 3H), 2.04 (br, 2H), 1.74-1.85 (m, 1H), 1.45 (br, 3H), 1.23-1.26 (m, 2H). MS (ESI) m/z (M+H)⁺480.1.

### Example 31

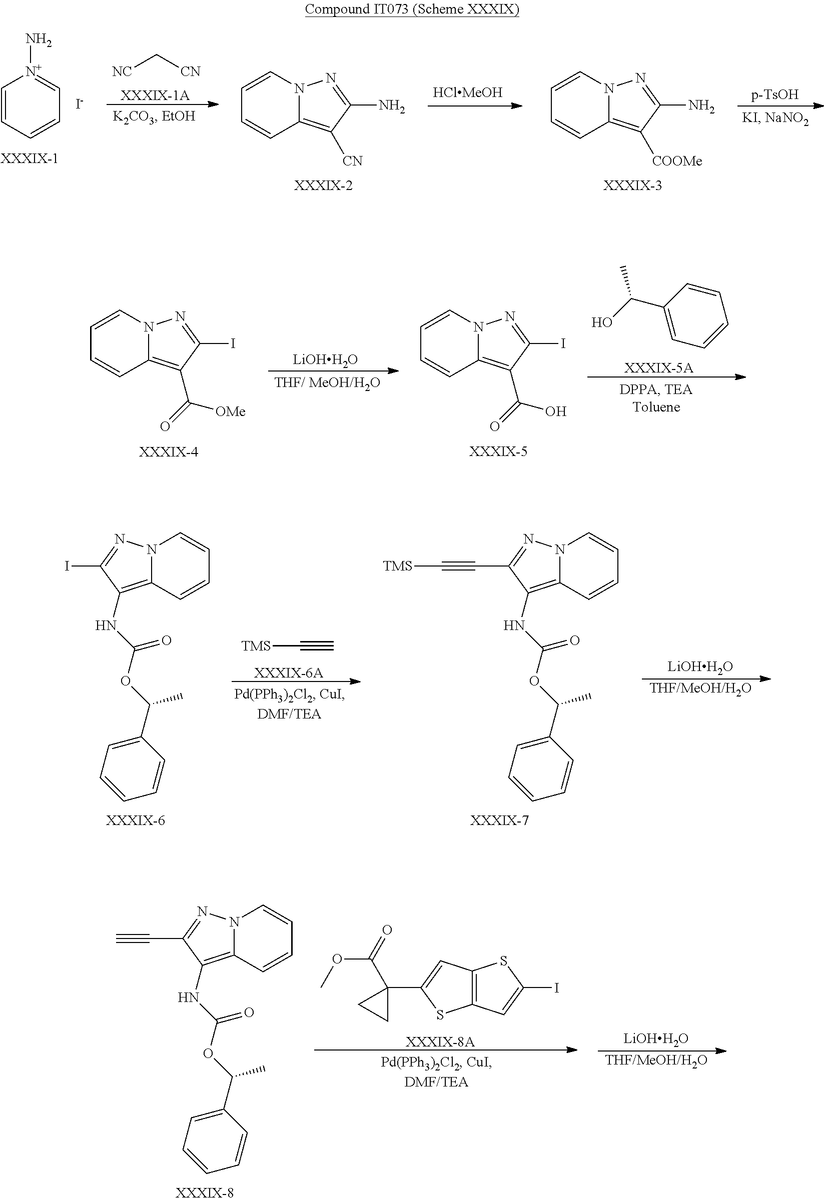

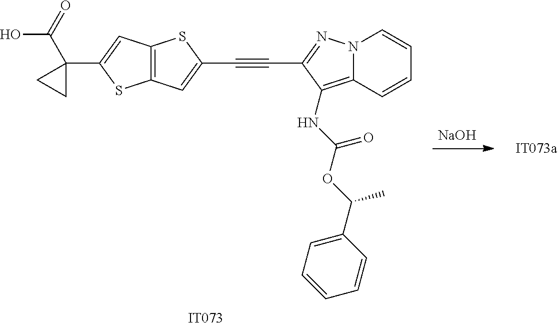 [SMILES: C[C@@H](OC(=O)Nc1c(C#Cc2cc3sc(C4(C(=O)O)CC4)cc3s2)nn2ccccc12)c1ccccc1 ; "IT073" ; "NaOH"]

To a solution of XXXIX-1 (10 g, 45 mmol) in EtOH (150 mL) was added XXXIX-1A (2.85 mL, 45 mmol) and K₂CO₃ (12.4 g, 90 mmol). The mixture was stirred at 90° C. for 24 h. After concentrated, the mixture was diluted with EtOAc, washed with brine, dried over Na₂SO₄, filtered and concentrated as XXXIX-2 (3 g, yield 42%).

A mixture of XXXIX-2 (1.1 g, 6.96 mmol) in HCl/MeOH (4N, 20 mL) was stirred at 80° C. for 24 h. After concentrated, the mixture was diluted with EtOAc, washed with brine, dried over Na₂SO₄, filtered and concentrated. The residue was purified by column (PE/EA=2/1) to afford XXXIX-3 (600 mg, yield 45.1%).

To a solution of p-TsOH.H₂O (1.79 g, 9.42 mmol) in MeCN (10 mL) was added XXXIX-3 (600 mg, 3.14 mmol). Then a solution of NaNO₂ (433 mg, 6.28 mmol) and KI (1.29 g, 7.85 mmol) in H₂O (2 mL) was gradually added. The reaction mixture was stirred for 3 h. Then the reaction mixture was then added H₂O, NaHCO₃ and Na₂S₂O₃. The precipitated aromatic iodide was filtered and by flash chromatography on silica gel (PE/EA=5/1) to afford XXXIX-4 (520 mg, yield 54.8%).

To a stirred solution of XXXIX-4 (600 mg, 2 mmol) in THF/MeOH/H₂O=1/1/1 (6 mL) was added LiOH.H₂O (420 mg, 10 mmol). After the addition, the solution was stirred overnight at rt. The solution was concentrated in vacuo, the aqueous layer was adjust pH to 2 with 1N HCl, and extracted with EtOAc. The organic layer was separated, dried and concentrated to afford crude XXXIX-5 (530 mg, crude), which was used to next step directly.

XXXIX-6, XXXIX-7, XXXIX-8, IT073 and its sodium salt IT073a were prepared following the similar procedure described in the synthesis of XII-5 and the alternative synthetic scheme XIII of IT017. IT073: MS (ESI) m/z (M+H)⁺ 527.9. IT073a: ¹H NMR (400 MHz, Methanol-d₄): δ8.42 (d, J=6.4 Hz, 1H), 7.23-7.47 (m, 8H), 7.07 (s, 1H), 6.94-6.97 (m, 1H), 5.87-5.89 (m, 1H), 1.62-1.65 (m, 5H), 1.21-1.23 (m, 2H). MS (ESI) m/z (M+H)⁺528.0.

### Example 32

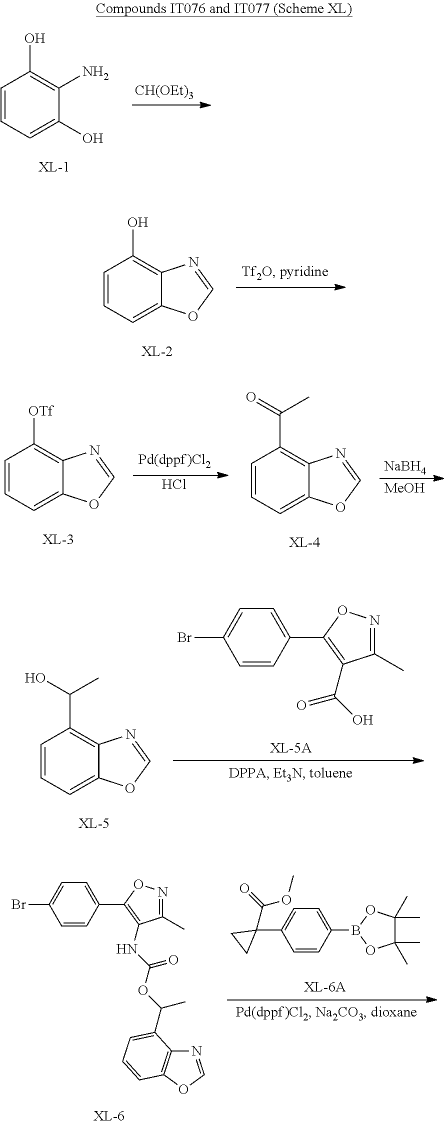

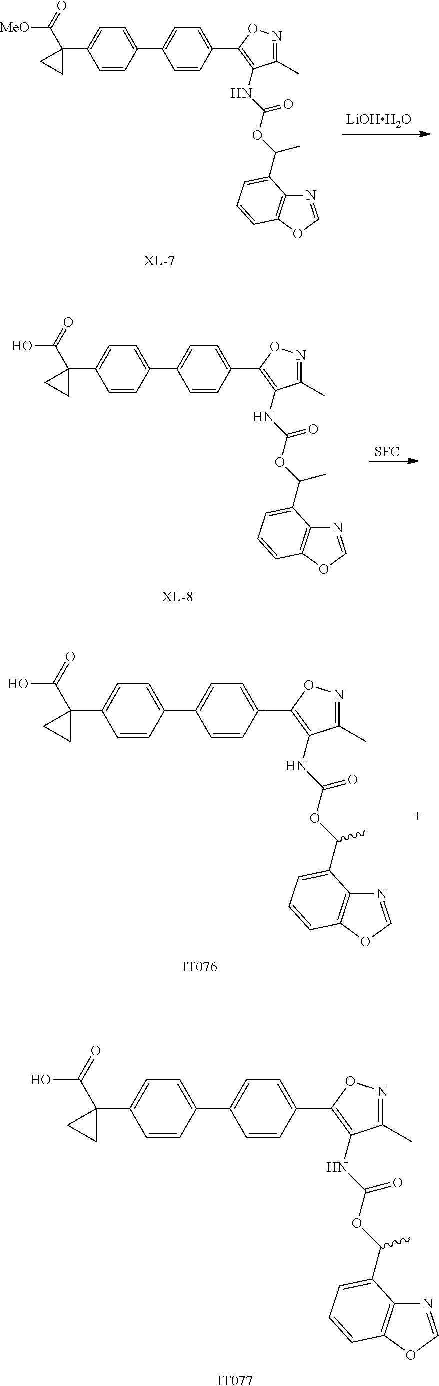

The solution of XL-1 (1 g, 8 mmol) in triethyl orthoformate (10 mL) was stirred at 130° C. for 2 hrs. Then the excess triethyl orthoformate was removed by evaporation. The residue was purified by column over silica gel (PE:EA=10/1) to afford XL-2 (0.68 g, yield 62%).

To a solution of XL-2 (500 mg, 3.7 mmol) in DCM (10 mL) was added trifluoromethanesulfonic anhydride (1.6 g, 5.6 mmol) and pyridine (585 mg, 7.4 mmol) at 0° C. The mixture was stirred at rt for 5 hrs. The mixture was diluted with water and extracted with EA. The organic layer was dried over Na₂SO₄, concentrated and purified by column over silica gel (PE:EA=10/1) to provide XL-3 (450 mg, yield: 46%).

A mixture of XL-3 (1.5 g, 5.6 mmol), tributyl (1-ethoxyvinyl)tin (2.3 g, 6.2 mmol), LiCl (24 mg, 0.56 mmol) and Pd(dppf)Cl₂ (0.3 g, 0.28 mmol) in dioxane (25 mL) was stirred at 100° C. for 4 hrs. The mixture was cooled to rt, then HCl (30 mL, 3N) and DCM (30 mL) was added. After stirred for 30 mins, the organic layer was separated, dried over Na₂SO₄, concentrated and purified by column over silica gel (PE:EA=3/1) to provide XL-4 (600 mg, yield 67%).

To a solution of XL-4 (200 mg, 1.24 mmol) in 5 mL of MeOH/H₂O (v/v=5/1) was added NaBH₄ (94 mg, 2.48 mmol) at 0° C. Then the mixture was stirred at 0° C. for 30 mins. Then NH₄Cl (aq, 2 mL) was added and most of MeOH was evaporated and the mixture was extracted with DCM. Then 5 mL of toluene was added and the volatile solvent DCM was concentrated at rt to afford XL-5 (1.24 mmol) which was used for next step directly.

To a solution of XL-5 (200 mg, 1.23 mmol) in toluene (10 mL) was added XL-5A (413 mg, 1.47 mmol), DPPA (404 mg, 1.47 mmol) and Et₃N (248 mg, 2.46 mmol) under nitrogen atmosphere. Then the mixture was heated to reflux for 2 hrs. Then most of toluene was evaporated. The residue was diluted with 3 mL of water and extracted with EA. The combined organic layers were washed with brine, dried over Na₂SO₄ and concentrated under vacuo. The crude product was purified by silica gel (PE:EA=1/1) to afford XL-6 (150 mg, yield 27.6%).

XL-7 and XL-8 were prepared following the similar procedure described in the synthesis of IT031. Enantiomers IT076 and IT077 were obtained from SFC separation of XL-8. IT076: ¹H NMR (Methanol-d₄, 400 MHz): δ 8.49 (s, 1H), 7.45-7.81 (m, 11H), 6.37 (br, 1H), 2.19 (s, 3H), 1.74 (d, J=6.0 Hz, 3H), 1.49 (br, 2H), 1.02 (br, 2H). MS (ESI) m/z (M+H)⁺524.2. IT077: ¹H NMR (Methanol-d₄, 400 MHz): δ 8.49 (s, 1H), 7.44-7.82 (m, 11H), 6.39 (br, 1H), 2.19 (s, 3H), 1.74 (d, J=6.0 Hz, 3H), 1.46 (br, 2H), 0.97 (br, 2H). MS (ESI) m/z (M+H)⁺524.2.

### Example 33

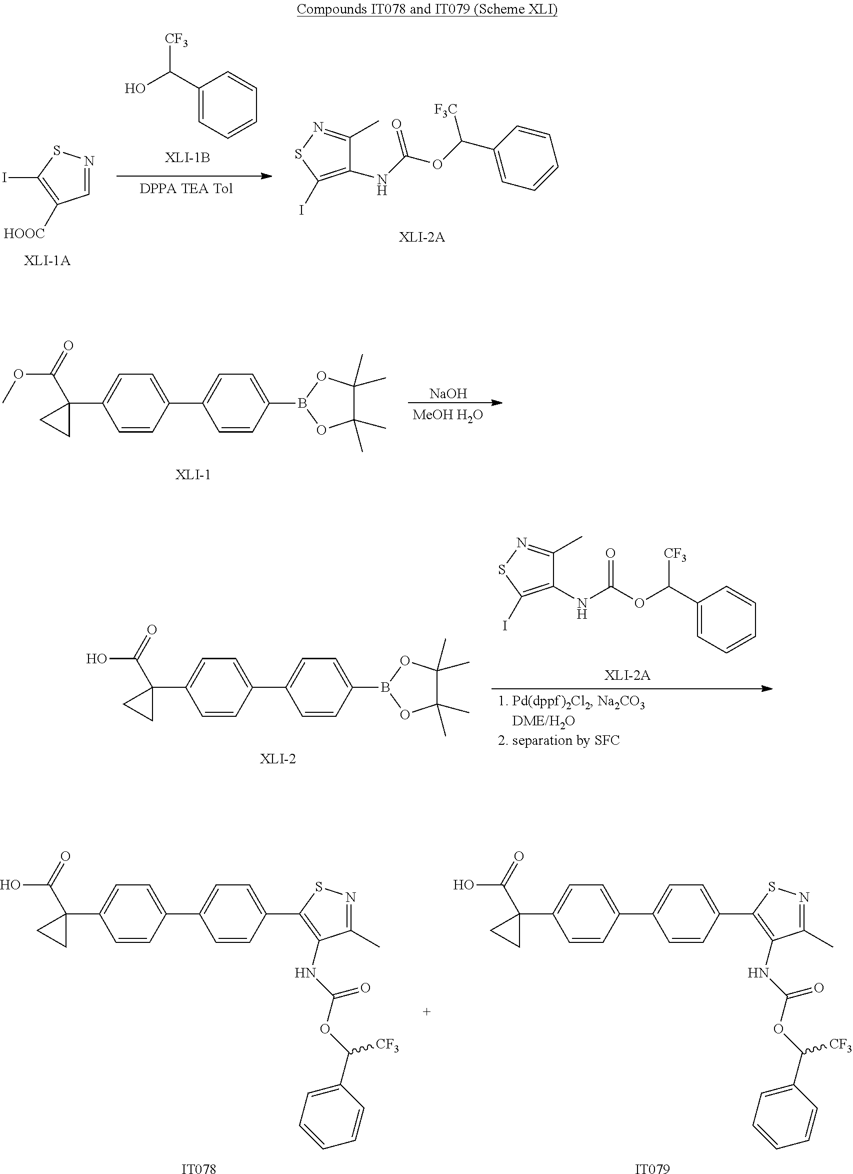

To a solution of XLI-1A (500 mg, 1.86 mmol) in dry toluene (10 mL) was added XLI-1B (393 mg, 2.23 mmol), triethylamine (373 mg, 3.72 mmol) and DPPA (611 mg, 2.23 mmol). The reaction mixture was heated to 80° C. for 3 h. The mixture was diluted with EtOAc, washed with brine, dried over Na₂SO₄, filtered and concentrated. The residue was purified by column (PE/EA=5/1) to give XLI-2A (800 mg, yield: 97%).

Enantiomers IT078 and IT079 were obtained by deprotection of XLI-1 with NaOH and subsequent Suzuki coupling with XLI-2A following the similar procedure described in the synthesis of III-5 followed by SFC separation. IT078: ¹H NMR (Methanol-d₄, 400 MHz): δ7.51-7.63 (m, 8H), 7.44-7.48 (m, 5H), 6.15-6.20 (m, 1H), 2.33 (s, 1H), 1.61-1.63 (m, 2H), 1.23-1.26 (m, 2H). MS (ESI) m/z (M+H)⁺553.1. IT079: ¹H NMR (Methanol-d₄, 400 MHz): δ7.51-7.62 (m, 8H), 7.44-7.48 (m, 5H), 6.15-6.20 (m, 1H), 2.33 (s, 1H), 1.60-1.63 (m, 2H), 1.23-1.26 (m, 2H). MS (ESI) m/z (M+H)⁺553.1.

### Example 34

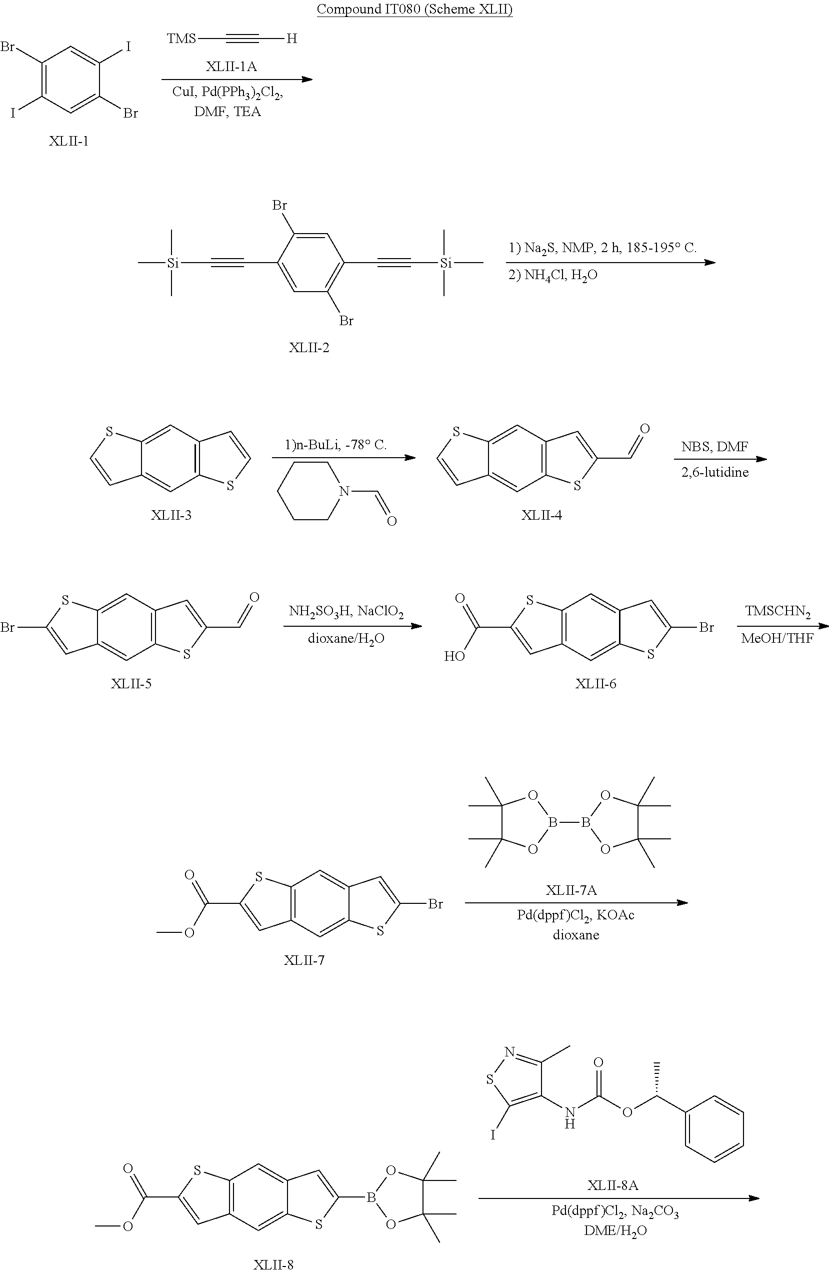

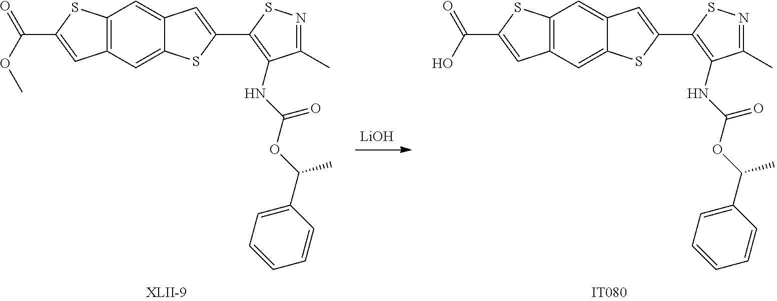

To a solution of XLII-1 (2 g, 4.1 mmol), CuI (78 mg, 0.41 mmol), and Pd(PPh₃)₂Cl₂ (287 mg, 0.41 mmol) in DMF (60 mL) and TEA (20 mL) (DMF was degassed through the solvent by bubbling N₂ for 15 min prior to use) was added XLII-1A (0.8 g, 8.2 mmol) dropwise at 0° C. After addition, the mixture was stirred at 4° C. for 12 h. The mixture was washed with H₂O, extracted with EtOAc. The organics were combined, dried with Na₂SO₄, filtered and concentrated. The residue was purified by column (PE) to afford XLII-2 (1.2 g, yield 68.6%).

To a stirred solution of Na₂S (2.7 g, 11.2 mmol) in NMP (72 mL) was added XLII-2 (1.2 g, 2.8 mmol). The mixture was heated at 185° C. for 2 h. The mixture was quenched with saturated NH₄Cl, extracted with EtOAc. The organics were combined, dried with Na₂SO₄, filtered and concentrated. The residue was purified by column (PE) to give XLII-3 (300 mg, yield 56%).

A solution of n-BuLi (2.5 M in hexane, 2.3 mL, 5.78 mmol) was added dropwise to suspension of XLII-3 (1.0 g, 5.26 mmol) in 25 mL of dry THF at −78° C. The mixture was stirred for 1.5 hours at −78° C. Then a solution of N-carbaldehyde (1.2 mL, 10.51 mmpl) in 2 mL of THF was added slowly. The mixture was stirred at −78° C. for 3 h then the temperature was slowly raise to rt and mixture was stirred overnight. The reaction mixture was quenched by addition of saturated NH₄Cl aqueous solution. The mixture was diluted with H₂O and extracted with EA. The combined organic layer was washed with brine, dried and concentrated. The residue was washed with TBME to afford—XLII-4 (0.9 g, yield 78%), which was used for next step directly.

N-bromosuccinimide (1.4 g, 7.87 mmol) was added in portions to a solution of XLII-4 (800 mg, 3.66 mmol) and 2,6-lutidine (400 mg, 3.73 mmol) in 30 mL of DMF. The mixture was heated to 60° C. and stirred overnight. The mixture was poured into 100 mL of H₂O. The precipitate was collected and dried in vacuum to afford XLII-5 (1.1 g, crude yield 100%) as a yellow solid, which was used for next step directly.

NH₂SO₃H (1.57 g, 14.13 mmol) was added to suspension of XLII-5 (700 mg, 2.36 mmol) in 24 mL of dioxane/H₂O (v/v=7/3). Then NaClO₂ (278 mg, 3.07 mmol) was added. The mixture was stirred for 3 hrs at rt. The mixture was poured in 30 mL of water. The precipitate was collected and purified by prep-HPLC to afford XLII-6 (90 mg, yield 12%).

A solution of (trimethylsilyl)diazomethane in hexane (2 N, 0.17 mL, 0.33 mmol) was added to a suspension of XLII-6 (70 mg, 0.22 mmol) in 1 mL of MeOH and 2 mL of THF. The mixture was stirred overnight at rt. Additional (trimethylsilyl)diazomethane (2 N in hexane, 0.17 mL, 0.33 mmol) was added and the mixture was further stirred for 5 hrs at rt. The mixture was concentrated to afford XLII-7 (70 mg, crude yield), which was used for next step directly.

XLII-8, XLII-9 and IT080 were prepared following the similar procedure described in the preparation of VI-6, VI-7 and IT001. IT080: ¹H NMR (400 MHz, Methanol-d₄): δ 8.57 (s, 1H), 8.28 (s, 1H), 8.16 (s, 1H), 7.84 (s, 1H), 7.17 (br, 5H), 5.67 (q, J=6.4 Hz, 1H), 2.42 (s, 3H), 1.38 (d, J=6.4 Hz, 3H). MS (ESI) m/z (M+H)⁺494.9.

### Example 35

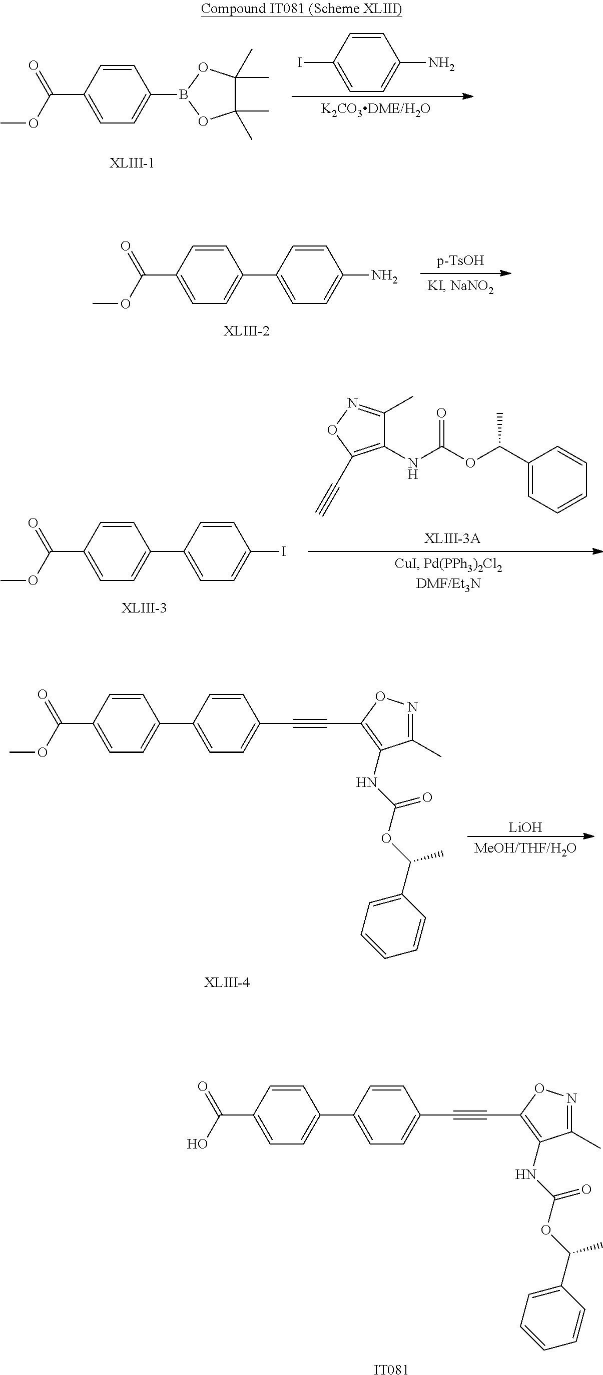

The mixture of XLIII-1 (3 g, 11.44 mmol), 4-iodoaniline (2.76 g, 12.59 mmol), Na₂CO₃ (2.46 g, 22.89 mmol) and Pd(dppf)Cl₂ in DME/H₂O (80 mL, v/v=3/1) was heated to reflux under nitrogen for overnight. After concentrated, the residue was partitioned between H₂O and DCM, and the aqueous phase was extracted with DCM. The combined organic layer was washed with brine, dried over Na₂SO₄, concentrated. The residue was purified by column (PE/EA=5/1) on silica gel to afford XLIII-2 (3 g, yield: 42.3%).

To a solution of p-TsOH.H₂O (2.76 g, 14.5 mmol) in MeCN (60 mL) was added XLIII-2. The resulting suspension of XLIII-2 (1.1 g, 4.84 mmol) salt was cooled to 10-15° C. and to the mixture was added, gradually a solution of NaNO₂ (0.84 g, 12.1 mmol) and KI (1.6 g, 9.69 mmol) in H₂O. The reaction mixture was stirred for 10 min then allowed to come 20° C. and stirred for 3 hrs. The reaction mixture was quenched with H₂O, NaHCO₃ and Na₂S₂O₃. The precipitated aromatic iodide was filtered and by flash chromatography (PE/EA=10/1) to afford XLIII-3 (500 mg, yield: 31.25%).

XLIII-4 and IT081 were prepared following the similar procedure described in the preparation of I-6 and IT001. IT081: MS (ESI) m/z (M+H)⁺466.9. Sodium salt IT081a: ¹H NMR (DMSO-d₆, 400 MHz): δ9.56 (s, 1H), 7.96 (d, J=7.6 Hz, 2H), 7.78 (d, J=8.4 Hz, 2H), 7.66 (d, J=7.6 Hz, 2H), 7.55 (d, J=8.0 Hz, 2H), 7.32-7.38 (m, 5H), 5.77 (q, J=6.0 Hz, 1H), 2.16 (s, 3H), 1.51-1.53 (d, J=6.0 Hz, 3H). MS (ESI) m/z (M+H)⁺466.9.

### Example 36

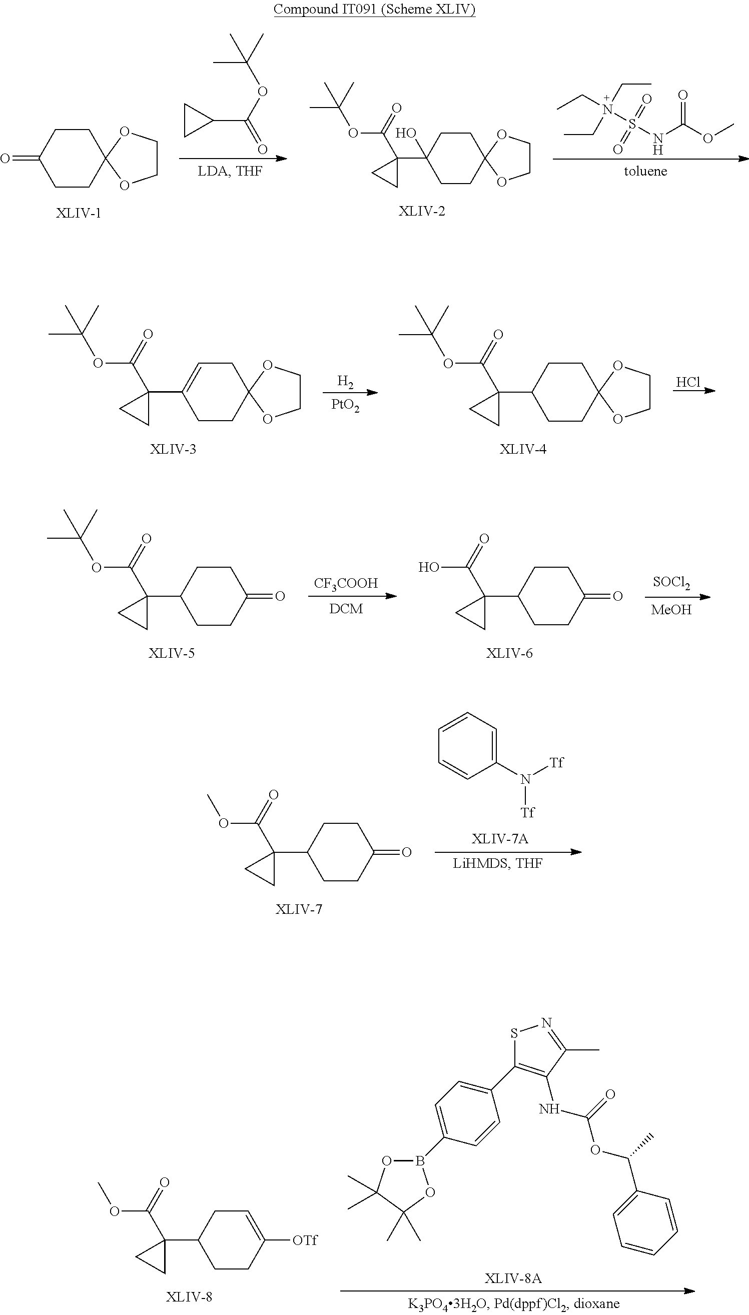

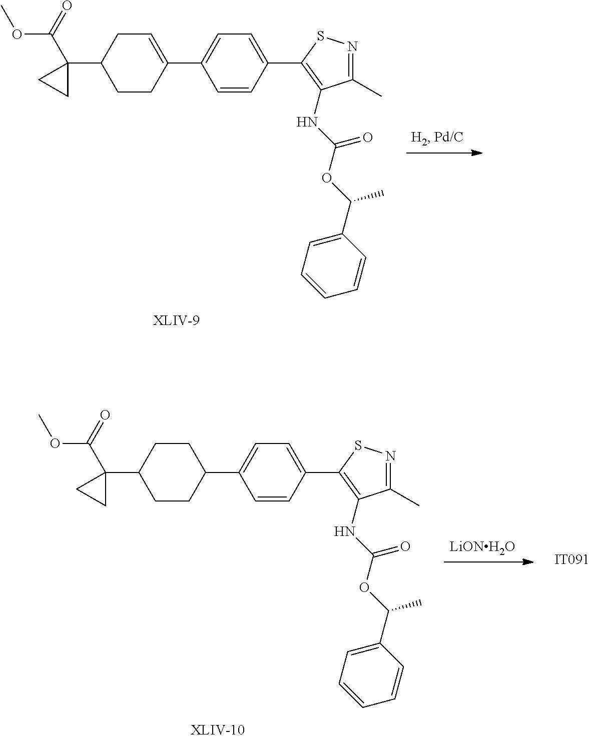

To a cooled (−78° C.) solution of 2M LDA in THF (1.4 mL, 2.8 mmol) was added tert-butyl cyclopropanecarboxylate (0.4 g, 2.8 mmol) in THF (5 mL). The mixture was stirred at −78° C. for 1 h. Then a solution of compound 1 (0.44 g, 2.8 mmol) in THF (5 mL) was added. The cooling bath was removed after stifling for 30 mins, the solution was stirred at rt for 3 h. The reaction was quenched with saturated NH₄Cl and the mixture was extracted with EA. The combined organic layers were washed and concentrated under vacuo. The residue was purified by column over silica gel (PE:EA=5/1) to give XLIV-2 (400 mg, yield 50%).

To a stirred solution of XLIV-2 (600 mg, 2 mmol) in toluene (10 mL) was added XLIV-2A (564 mg, 2.4 mmol) under N₂. The mixture was heated to reflux for 2 h. After being cooled to rt, the mixture was diluted with water and extracted with EA. The combined organic layers were washed and concentrated under vacuo. The residue was purified by column on silica gel (PE:EA=10/1) to give XLIV-3 (400 mg, yield 71%).

To a stirred solution of XLIV-3 (150 mg, 0.54 mmol) in EA (10 mL) was added PtO₂ (50 mg, 33%). Then the suspension was degassed under vacuum and purged with H₂ (50 psi) at 30° C. for 1 h. Then the solution was filtered and evaporated in vacuo to give XLIV-4 (100 mg, yield 67%).

To a solution of XLIV-4 (500 mg, 1.8 mmol) in MeOH (10 mL) was added HCl (5 mL, 6 N). Then it was stirred at rt for 2 h. The mixture was diluted with water and extracted with DCM. The organic layers were washed with brine, and concentrated under vacuo to give XLIV-5 (400 mg, yield 95.6%).

To a solution of XLIV-5 (350 mg, 1.48 mmol) in DCM (4 mL) was added CF₃COOH (4 mL). Then it was stirred at rt for 2 h. The mixture was diluted with water and extracted with DCM. The organic layers were washed with brine, and concentrated under vacuo to give XLIV-6 (250 mg, yield 88%).

To a solution of XLIV-6 (300 mg, 1.56 mmol) in MeOH (10 mL) was added SOCl₂ (187 mg, 1.56 mmol). Then it was stirred at 30° C. overnight. The mixture was diluted with water and extracted with DCM. The organic layers were washed with brine, and concentrated under vacuo to give XLIV-7 (150 mg, yield 47%).

To a solution of XLIV-7 (150 mg, 0.72 mmol) in THF (5 mL) was added LiHMDS (0.81 mL, 0.81 mmol) at −78° C. The solution was stirred at −78° C. for 1 h. Then XLIV-7A (293 mg, 0.81 mmol) in THF (5 mL) was added. The cooling bath was removed after stirring for 30 mins, the solution was stirred at rt overnight. The reaction was quenched with saturated aq. NH₄Cl and the mixture was extracted with EA. The combined organic layers were washed with brine, and concentrated under vacuo. The residue was purified by column on silica gel (PE:EA=20/1) to give XLIV-8 (200 mg, yield 84%).

To a stirred solution of XLIV-8 (160 mg, 0.47 mmol), XLIV-8A (241 mg, 0.52 mmol), K₃PO₄.3H₂O (250 mg, 0.94 mmol) in dioxane (10 mL) was added Pd(dppf)Cl₂ (34.4 mg, 0.047 mmol) under nitrogen atmosphere. The mixture was purged with N₂ for 5 mins and heated to reflux for 4 h. After cooled, the mixture was diluted with water and extracted with DCM. The combined organic layers were washed with brine, and concentrated under vacuo. The residue was purified by column on silica gel (PE:EA=5/1) to give XLIV-9 (140 mg, yield 57%).

To a stirred solution of XLIV-9 (130 mg, 0.25 mmol) in EA (10 mL) was added Pd/C (65 mg, 50%). Then the suspension was degassed under vacuum and purged with H₂ (50 psi) at rt for 2 h. Then the solution was filtered and evaporated in vacuo to give XLIV-10 (110 mg, yield: 67%).

IT091 was obtained by LiOH hydrolysis of XLIV-10 (7.5 mg, yield: 7.7%). MS (ESI) m/z (M+H)⁺505.2. ¹H NMR (CDCl₃, 400 MHz): δ 7.20-7.39 (m, 7H), 7.12-7.14 (d, J=8.0 Hz, 2H), 6.06 (br, 1H), 5.78 (br, 1H), 2.99 (br, 1H), 2.30 (s, 3H), 2.12-2.15 (m, 2H), 1.71-1.84 (m, 3H), 1.32-1.49 (m, 7H), 1.09-1.19 (m, 2H), 0.68-0.76 (m, 2H).

IT092 was prepared by Suzuki Coupling of XLIV-8 with XIII-9 using the similar procedure described in the alternative synthesis of XIII-6, followed by standard LiOH hydrolysis. ¹H NMR (Methanol-d₄, 400 MHz): δ 7.47 (s, 1H), 7.32-7.38 (m, 5H), 6.00 (s, 1H), 5.83-5.85 (m, 1H), 3.66 (s, 3H), 2.12-2.23 (m, 4H), 1.70-1.77 (m, 1H), 1.51-1.59 (m, 3H), 1.31-1.33 (m, 2H), 1.16 (m, 2H), 0.80 (m, 2H). MS (ESI) m/z (M+H)⁺434.2.

### Example 37

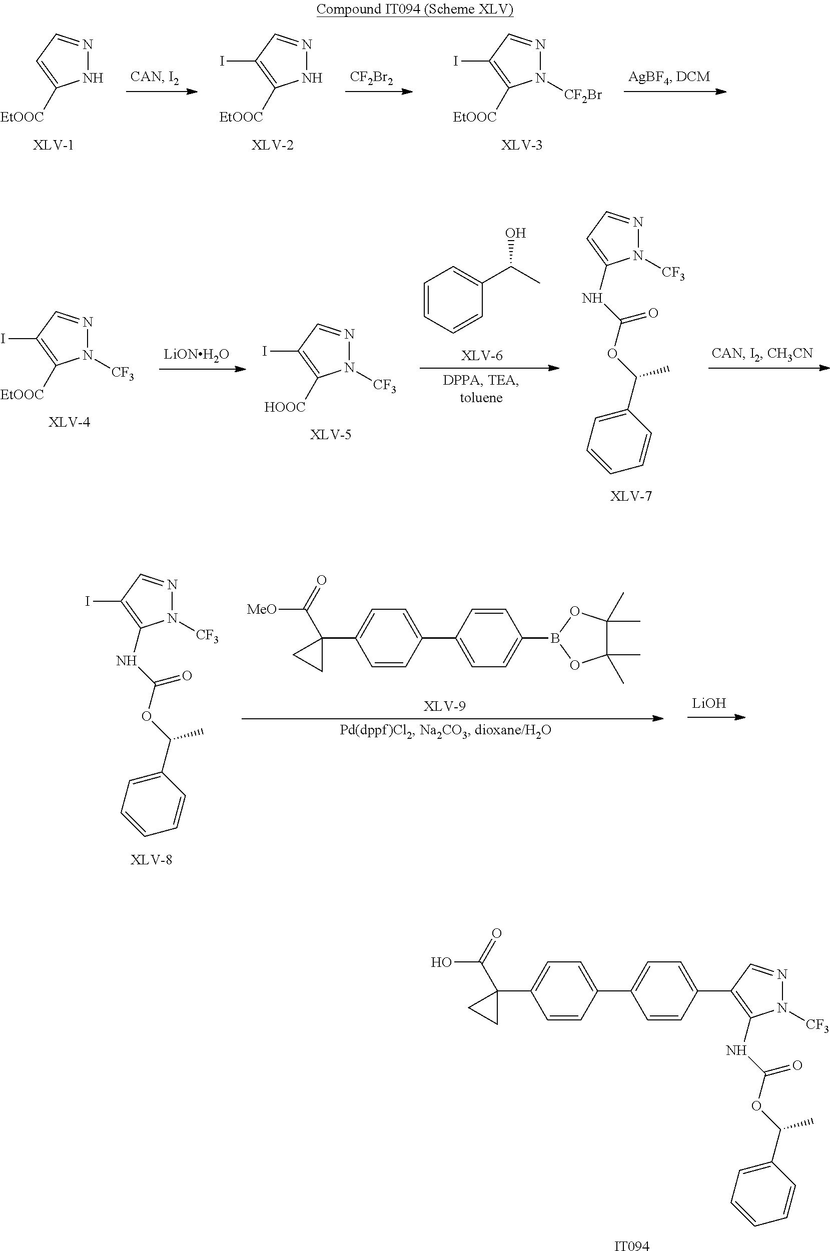

The solution of XLV-1 (10 g, 71.4 mmol), CAN (39.1 g, 71.4 mmol), I₂ (18 g, 71.4 mmol) in CH₃CN (182 mL) was stirred at 25° C. for 15 h. Then the solution of NaHSO₃ was added until the mixture was light yellow, then extracted with EtOAc, dried over Na₂SO₄, concentrated and purified by column (PE/EA=10/1) to provide XLV-2 (16 g, yield: 84%) as a white solid.

To a stirred solution of XLV-2 (20 g, 75.2 mmol) in DMF (250 mL) was added NaH (4.5 g, 112.8 mmol) at 0° C. After 1 h, CF₂Br₂ (31.3 g, 150.4 mmol) was added, then the mixture was stirred at 25° C. overnight. The mixture was quenched with water and extracted with EtOAc. The organic layer was dried over Na₂SO₄, concentrated under vacuo and purified by column (PE/EA=50/1) to provide XLV-3 (2 g, yield: 6.8%).

To a stirred solution of XLV-3 (2 g, 5.07 mmol) in DCM (30 mL) was added AgBF₄ (1.8 g, 10.14 mmol) at −78° C. Then the solution was stirred at rt for 10 h. The mixture was diluted with DCM, filtered, concentrated and purified by flash column (PE/EA=3/1) to provide XLV-4 (1.9 g, yield: 97%).

To a stirred solution of XLV-4 (1.9 g, 5.69 mmol) in MeOH/H₂O (24 mL/4 mL) was added LiOH.H₂O (1.4 g, 34.13 mmol). The mixture was stirred at rt for 30 mins. Then MeOH was removed, HCl (6N) was added to adjust pH <3, and extracted with EtOAc. The organic layer was separated, dried and concentrated to provide XLV-5 (1.5 g, yield: 88%).

A mixture of XLV-5 (100 mg, 0.33 mmol), XLV-6 (48 mg, 0.39 mmol), DPPA (107 mg, 0.39 mmol) and TEA (67 mg, 0.66 mmol) in toluene (5 mL) was stirred at 90° C. for 3 h. The toluene was removed and diluted with EtOAc and washed with water. The organic layer was dried over Na₂SO₄, concentrated and purified by column (PE/EA=3/1) to provide XLV-7 (80 mg, yield: 81%).

To a stirred solution of XLV-7 (25 mg, 0.14 mmol) in CH₃CN (5 mL) was added CAN (74 mg, 0.14 mmol) and I₂ (35 mg, 0.14 mmol). The mixture was stirred at rt for 5 h. NaHSO₃ (aq.) was added to quench the solution until the solution turned light yellow, extracted with EtOAc. The organic layer was dried over Na₂SO₄, concentrated and purified by column (PE/EA=5/1) to provide XLV-8 (25 mg, yield: 40%).

IT094 was obtained by Suzuki-Coupling of XLV-8 with XLV-9, followed by LiOH hydrolysis. IT094: MS (ESI) m/z (M+H)⁺536.2. Sodium salt IT094a: MS (ESI) m/z (M+H)⁺536.2. ¹H NMR (DMSO-d₆, 400 MHz): δ 8.29 (s, 1H), 7.59 (br, 4H), 7.50-7.52 (m, 2H), 7.34-7.36 (m, 7H), 5.74 (br, 1H), 1.46 (br, 3H), 1.24 (br, 2H), 0.78 (br, 2H).

### Example 38

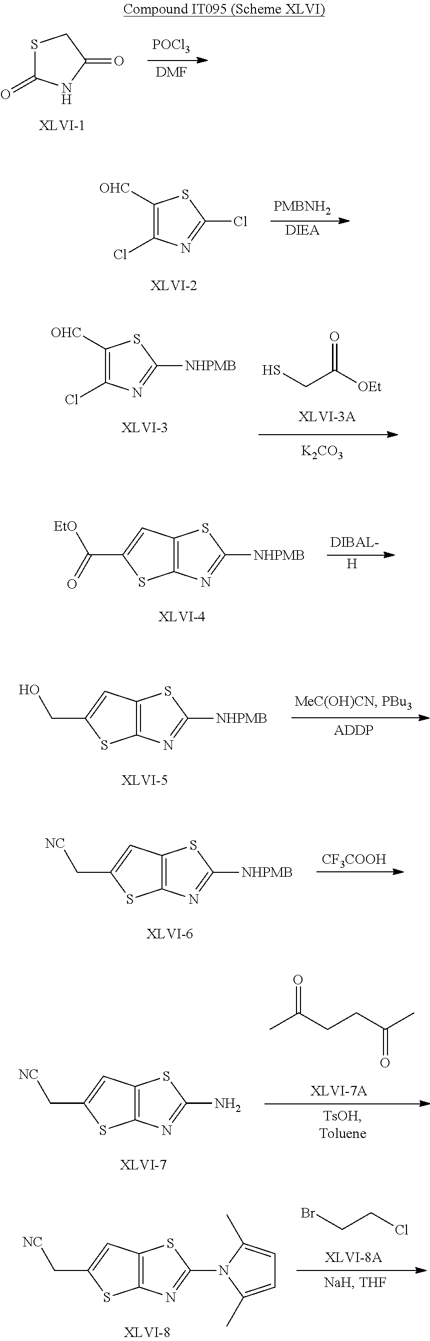

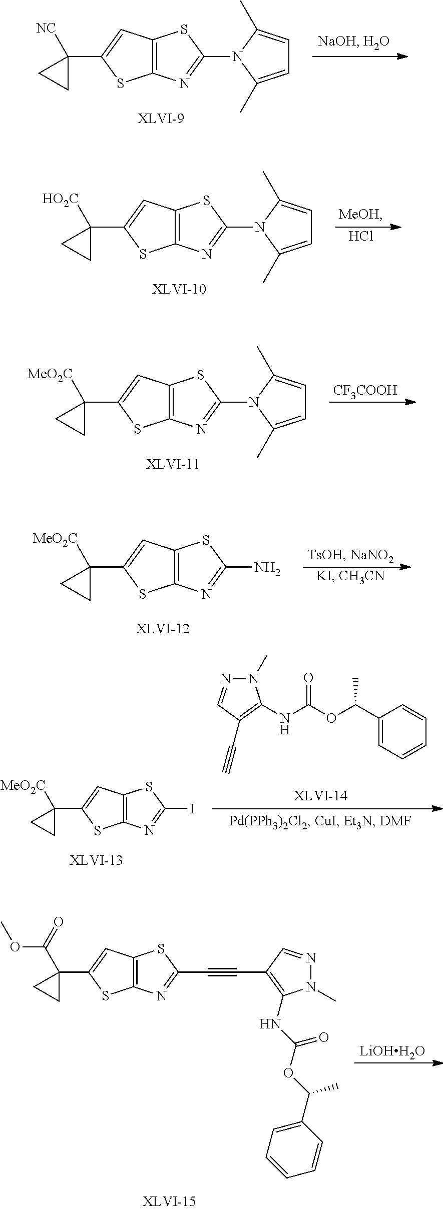

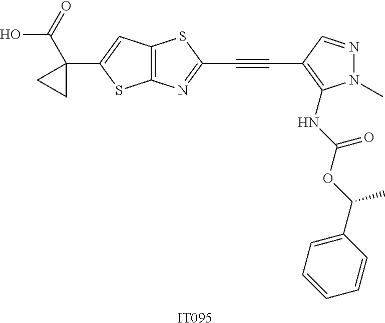 [SMILES: C[C@@H](OC(=O)Nc1c(C#Cc2nc3sc(C4(C(=O)O)CC4)cc3s2)cnn1C)c1ccccc1 ; "IT095"]

DMF (68.4 g, 940.2 mmol) was added dropwise to a suspension of XLVI-1 (100 g, 854.7 mmol) in POCl₃ (476 mL, 780 g, 1.71 mol) at 0° C. Then the mixture was stirred for 1 h at rt, then for 1 h at 85° C., after which the mixture was refluxed for 2 h. POCl₃ was removed in vacuum and the mixture was poured onto water, then extracted with DCM. The organic layer was washed with brine, dried over Na₂SO₄, concentrated in vacuum. The residue was purified by column on silica gel (PE/EA=50/1) to afford XLVI-2 (47 g, yield: 30%).

The solution of PMBNH₂ (88.5 g, 650 mmol), DIEA (226.5 mL) in THF (800 mL) was slowly added XLVI-2 (117.5 g, 650 mmol) in THF (400 mL) at rt. The mixture was stirred overnight at rt under nitrogen. THF was removed in vacuum and the mixture was washed with water and EtOAc, then filtered through a Celite pad to afford XLVI-3 (130 g, yield: 71%) without further purification.

The solution of XLVI-3 (100 g, 354.6 mmol) and K₂CO₃ (146.8 g, 1.06 mol) in DMF (1000 mL) was added XLVI-3A (51.1 g, 425.5 mmol) at rt. The mixture was heated to 120° C. and stirred for 4 hs at that temperature under nitrogen. DMF was removed in vacuum and the mixture was washed with water and EtOAc. The combined organic layers were dried over Na₂SO₄, and concentrated under reduced pressure and purified by column chromatography on silica gel (PE/EA=30/1-5/1) to afford XLVI-4 (77 g, yield: 62%).

The solution of XLVI-4 (30 g, 86.2 mmol) in 1500 mL of DCM was added DIBAL-H (431 mL, 43 μmol) at −78° C., and stirred at that temperature for 1 h under nitrogen. The mixture was quenched with NaHCO₃ (aq, 500 mL) and was diluted with EtOAc. The solution was stirred at rt for 20 mins and the resulting mixture was filtered through a Celite pad to afford XLVI-5 (23.5 g, crude yield: 90%), which used for next step without further purification.

To a solution of XLVI-5 (90 g, 294.1 mmol) and ADDP (81.5 g, 323.5 mmol) in THF (2700 mL) were added MeC(OH)CN (38.2 mL) and PBu₃ (132.5 mL) at 0° C. The mixture was stirred for 1 h at rt. The mixture was quenched with water and EtOAc. The combined organic layers were dried over Na₂SO₄, and concentrated under vacuo. The residue was purified by column chromatography on silica gel (PE/DCM=3/1-1/1) to afford XLVI-6 (30 g, yield: 32.4%).

To a solution of XLVI-6 (20 g, 63.5 mmol) in TFA (200 mL) was heated to 55° C. and stirred at that temperature for 5 h. The mixture was adjusted to pH=7-8 with NaHCO₃ (aq.) and extracted with DCM. The organic layer were washed with brine, dried over Na₂SO₄, concentrated in vacuum to afford XLVI-7 (10 g, yield: 81%), which used for next step without further purification.

To a stirred solution of XLVI-7 (11 g, 57 mmol) in toluene (400 mL) was added XLVI-7B (6.5 g, 7.81 mL, 57 mmol) and p-TsOH (541.5 mg, 2.85 mmol). After the addition, the solution was heated to 130° C. for 5 hours. Toluene was removed in vacuum and the residue was purified by column chromatography on silica gel (PE/DCM=3/1) to afford XLVI-8 (9.8 g, yield: 63%).

The solution of XLVI-8 (4.9 g, 18 mmol) in THF (60 mL) was slowly added NaH (1.4 g, 35.9 mmol) at 0° C. The mixture was stirred at rt for 1.5 h under nitrogen. Then XLVI-8A (3.4 g, 23.3 mmol) was added at 0° C. The mixture was stirred at rt for 2 h under nitrogen. The mixture was quenched with NH₄Cl (aq.), and extracted with EtOAc. The combined organic layers were dried over Na₂SO₄, and concentrated under vacuo. The residue was purified by column chromatography on silica gel (PE/EA=9/1) to afford XLVI-9 (4.5 g, yield: 83%).

To a stirred solution of XLVI-9 (2.2 g, 7.4 mmol) in MeOH (60 mL) was added NaOH (120 mL, 35% w). After the addition, the solution was heated to 85° C. overnight. MeOH was removed in vacuum and the mixture was adjusted to pH=4-5 with 4M HCl, and was extracted DCM. The organic layer was washed with brine, dried over Na₂SO₄, concentrated in vacuum to afford XLVI-10 (2.8 g, crude yield: 116%), which used for next step without further purification.

To a stirred solution of XLVI-10 (2.8 g, 8.8 mmol) in MeOH (50 mL) was added HCl (300 mg, 12M). After the addition, the solution was heated to 80° C. overnight. MeOH and HCl was removed in vacuum and the residue was purified by column chromatography on silica gel (PE/EA=9/1) to afford c XLVI-11 (1.7 g, yield: 58%).

To a solution of XLVI-11 (3.4 g, 10.2 mmol) in H₂O (40 mL) was slowly added TFA (40 mL) at 0° C. The mixture was heated to 60° C. and stirred at that temperature for 3 h. The mixture was adjusted to pH=8 with NaHCO₃ (aq.) and was extracted DCM. The organic layers were washed with brine, dried over Na₂SO₄, concentrated in vacuum to afford XLVI-12 (4.0 g, crude yield: 153%).

To a stirred solution of XLVI-12 (4.0 g, 15.7 mmol) in MeCN (50 mL) was added TsOH (9.0 g, 47.2 mmol). Then NaNO₂ (2.1 g, 31.4 mmol), KI (6.5 g, 39.3 mmol) dissolved in H₂O (30 mL) was added dropwise at 0° C. After the addition, the solution was stirred at rt for 4 h. MeCN was removed in vacuum and the reaction mixture was extracted EtOAc. The organic layers were washed with brine, dried over Na₂SO₄, concentrated in vacuum. The residue was purified by column chromatography on silica gel (PE/EA=10/1) to afford XLVI-13 (1.7 g, yield: 30%).

XLVI-15 and IT095 were prepared following the similar procedure described in the preparation of I-6 and IT001. IT095: MS (ESI) m/z (M+H)⁺493.1. ¹H NMR (Methanol-d₄, 400 MHz): δ 7.66 (s, 1H), 7.35-7.37 (m, 2H), 7.26-7.29 (m, 2H), 7.19-7.21 (m, 2H), 5.80-5.85 (q, J=6.4 Hz, 1H), 3.67 (s, 1H), 1.73-1.76 (m, 2H), 1.56-1.58 (d, J=6.4 Hz, 3H), 1.44-1.46 (m, 2H).

IT102 was prepared by following the similar procedure described in the synthesis of IT095 using (R)-1-phenylethyl (5-ethynyl-3-methylisoxazol-4-yl)carbamate in place XLVI-14 in the Suzuki-Coupling with XLVI-13. ¹H NMR (Methanol-d₄, 400 MHz): δ 7.25-7.42 (m, 6H), 5.86 (d, J=6.4 Hz, 1H), 2.24 (s, 3H), 1.81 (brs, 2H), 1.59-1.61 (d, J=6.4 Hz, 3H), 1.51 (brs, 2H). MS (ESI) m/z (M+H)⁺ 494.2.

### Example 39

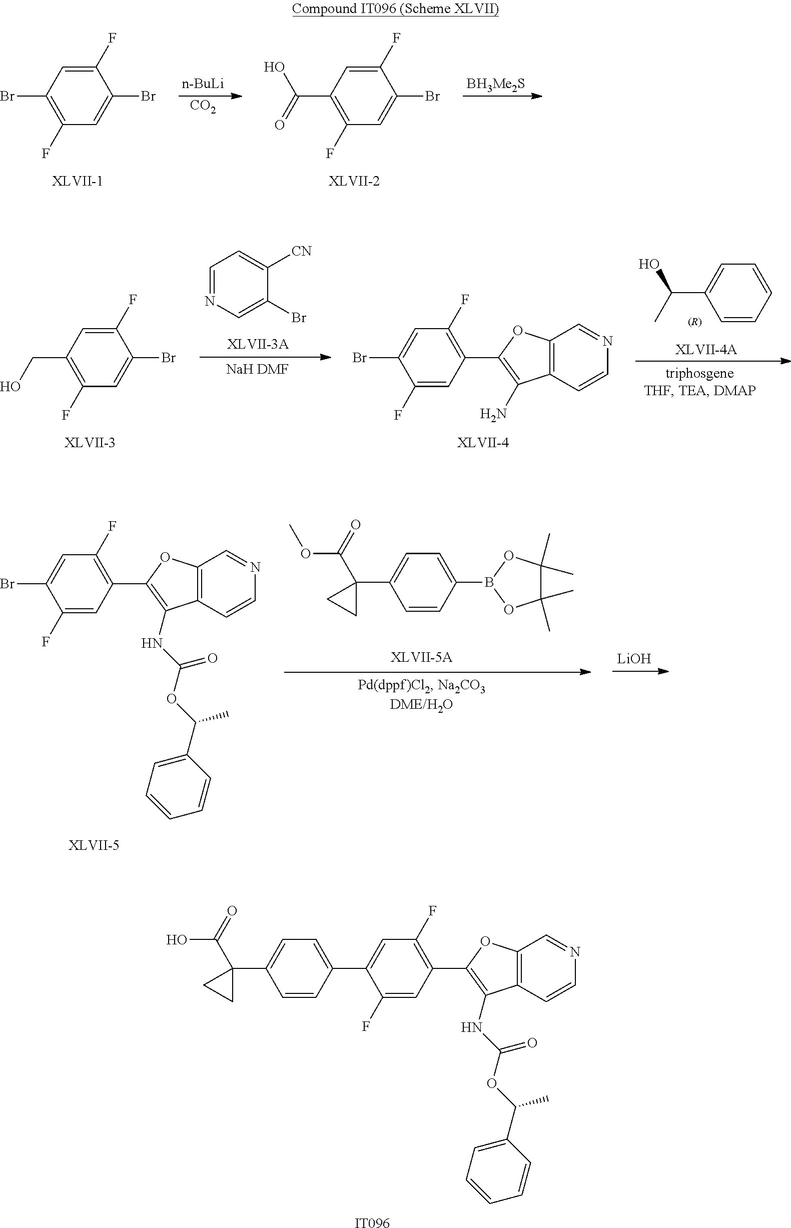

To the solution of XLVII-1 (13.55 g, 50 mmol), in Et₂O (150 mL) was added n-BuLi (2.5 N, 20 mL) at −78° C. The reaction mixture was stirred at −78° C. under Ar for 30 min and CO₂ was bubbled into the solution. The mixture was warmed up to rt. The precipitate was collected by filtration and washed with Et₂O. The obtained solid was treated with water and HCl (1N) to pH=2. The mixture was extracted with t-BuOMe. The combined organic layers were washed with brine, dried over MgSO₄, and concentrated to afford XLVII-2 (10.0 g, yield 84.4%), which was used next step without purification.

The mixture of XLVII-2 (2.37 g, 10 mmol) in THF (25 mL) was added BH₃.Me₂S (10 N, 2.5 mL) at rt under N₂, The mixture was heated to reflux for 2 h and quenched with adding MeOH and diluted with EtOAc. The organic layer was washed with brine, dried over MgSO₄ and concentrated under vacuo then purified by chromatography on silica gel (PE/EA=3/1) to afford XLVII-3 (1.50 g, yield 67.3%).

NaH (210 mg, 8.8 mmol) was added to a solution of XLVII-3 (446 m g, 2 mmol) in DMF (10 mL) at 0° C. The reaction mixture was stirred at 0° C. for 30 mins. A solution of XLVII-3A (366 mg, 2 mmol) in DMF (5 mL) was added dropwise. The reaction mixture was stirred at 0° C. for 4 h. Water (5 mL) was added. The reaction mixture was diluted with brine and EtOAc. The aqueous layer was extracted with EtOAc. The combined organic layer was washed with brine, dried over MgSO₄ and concentrated. The crude product was purified by column (PE/EA=2/1) to afford XLVII-4 (200 mg, yield 30.7%).

XLVII-5 and IT096 were prepared following the similar procedure described in the preparation of XXI-3 and IT031. IT096: ¹H NMR (Methanol-d₄, 400 MHz): δ 9.16 (s, 1H), 8.51 (s, 1H), 7.95 (s, 1H), 7.70-7.73 (m, 1H), 7.60 (d, J=7.2 Hz, 2H), 7.53 (d, J=8.4 Hz, 2H), 7.40-7.44 (m, 4H), 7.31 (br, 2H), 5.83-5.84 (m, 2H), 1.65-1.67 (m, 5H), 1.27-1.30 (m, 2H). MS (ESI) m/z (M+H)⁺555.1.

### In Vitro Assays

Establishment of a CHO Cell Line Stably Expressing Human LPA₁

A 1.1 kb cDNA encoding the human LPA₁ receptor is cloned from human lung. Human lung RNA (Clontech Laboratories, Inc. USA) is reverse transcribed using the RETROscript kit (Ambion, Inc.) and the full-length cDNA for human LPA₁ is obtained by PCR of the reverse transcription reaction. The nucleotide sequence of the cloned human LPA₁ is determined by sequencing and is confirmed to be identical to the published human LPA₁ sequence (An et al. Biochem. Biophys. Res. Commun. 231:619 (1997). The cDNA is cloned into the pCDNA5 pcDNA5/FRT expression plasmid and is transfected in CHO cells using lipofectamine 2000 (Invitrogen Corp., USA). Clones stably expressing human LPA₁ are selected using hygromycin and identified as cells that show Ca-influx in response to LPA.

Generation of Cells Transiently Expressing Human LPA₂

A vector containing the human LPA₂ receptor cDNA is obtained from the Missouri S&T cDNA Resource Center (www.cdna.org). The full-length cDNA fragment for human LPA₂ is obtained by PCR from the vector. The nucleotide sequence of the cloned human LPA₂ is determined by sequencing and is confirmed to be identical to the published human LPA₂ sequence (NCBI accession number NM_{—}004720). The cDNA is cloned into the pCDNA3 pcDNA3.1 expression plasmid and is transfected into B103 cells (Invitrogen Corp., USA) by seeding cells in a 96-well poly-D-lysine coated plate at 30,000-35,000 cells per well together with 0.2 μl lipofectamine 2000 and 0.2 μg of the LPA₂ expression vector. Cells are cultured overnight in complete media before being assayed for LPA-induced Ca-influx.

Establishment of a CHO Cell Line Stably Expressing Human LPA₃

A vector containing the human LPA₃ receptor cDNA is obtained from the Missouri S&T cDNA Resource Center (www.cdna.org). The full-length cDNA fragment for human LPA₃ is obtained by PCR from the vector. The nucleotide sequence of the cloned human LPA₃ is determined by sequencing and is confirmed to be identical to the published human LPA₃ sequence (NCBI accession number NM_{—}012152). The cDNA is cloned into the pCDNA5 pcDNA5/FRT expression plasmid and is transfected in CHO cells using lipofectamine 2000 (Invitrogen Corp., USA). Clones stably expressing human LPA₃ are selected using hygromycin and identified as cells that show Ca-influx in response to LPA.

LPA1 and LPA3 Calcium Flux Assays

Human LPA₁ or LPA₃ expressing CHO cells were seeded at 20,000-45,000 cells per well in a 96-well poly-D-lysine coated plate one or two days before the assay. Prior to the assay, the cells were washed once with PBS and then cultured in serum-free media overnight. On the day of the assay, a calcium indicator dye (Calcium 4, Molecular Devices) in assay buffer (HBSS with Ca²⁺ and Mg²⁺ and containing 20 mM Hepes and 0.3% fatty-acid free human serum albumin) was added to each well and incubation continued for 1 hour at 37° C. 10 μl of test compounds in 2.5% DMSO were added to the cells and incubation continued at room temperature for 30 minutes. Cells were then stimulated by the addition of 10 nM LPA and intracellular Ca²⁺ measured using the Flexstation 3 (Molecular Devices). IC₅₀, were determined using Graphpad prism analysis of drug titration curves.

LPA2 Calcium Flux Assay

Following an overnight culture with lipofectamine 2000 and the LPA₂ expression vector, the B103 cells are washed once with PBS then serum starved for 4 hours. A calcium indicator dye (Calcium 4, Molecular Devices) in assay buffer (HBSS with Ca²⁺ and Mg²⁺ and containing 20 mM Hepes and 0.3% fatty-acid free human serum albumin) is added to each well and incubation continued for 1 hour at 37° C. 10 μl of test compounds in 2.5% DMSO are added to the cells and incubation continued at room temperature for 30 minutes. Cells are the stimulated by the addition of 10 nM LPA and intracellular Ca²⁺ measured using the Flexstation 3 (Molecular Devices). IC₅₀, are determined using Graphpad prism analysis of drug titration curves.

GTPγS Binding Assay

The ability of a compound to inhibit binding of GTP to LPA₁ is assessed via a membrane GTPγS assay. CHO cells stably expressing the recombinant human LPA₁ receptor are resuspended in 10 mM Hepes, 7.4 containing 1 mM DTT, lysed and centrifuged at 75,000×g to pellet the membranes. The membranes are resuspended in 10 mM Hepes, 7.4 containing 1 mM DTT and 10% glycerol. Membranes (˜(−25 μg per well) are incubated in 96-well plates with 0.1 nM [³⁵S]-GTPγS, 900 nM LPA, 5 μM GDP, and test compound in Assay Buffer (50 mM Hepes, pH 7.4, 100 mM NaCl, 10 mM MgCl₂, 50 μg/ml saponin and 0.2% fatty-acid free human serum albumin) for 30 minutes at 30° C. The reactions are terminated by rapid filtration through Whatman GF/B glass fiber filter plates. The filter plates are washed 3 times with 1 ml cold Wash Buffer (50 mM Hepes, 7.5, 100 mM NaCl and 10 mM MgCl₂) and dried. Scintillant is then added to the plates and the radioactivity retained on the filters is determined on a Packard TopCount (Perkin Elmer). Specific binding is determined as total radioactive binding minus non-specific binding in the absence of the ligand (900 nM LPA). IC₅₀, are determined using Graphpad prism analysis of drug titration curves.

Beta-Arrestin Based Assays for Human LPA1R Antagonists and Agonists

A CHO cell line stably expressing the ProLink™ tagged human LPA1R was obtained from DiscoverX Inc, Fremont, Calif. In this system, β-Arrestin was fused to an N-terminal deletion mutant of β-galactosidase (termed the enzyme acceptor or EA), the human LPA1R was fused to a smaller (42 amino acids) weakly complementing fragment termed ProLink™. In cells that stably express these fusion proteins, agonist/ligand stimulation resulted in the interaction of β-Arrestin and the ProLink-tagged GPCR, forcing the complementation of the two β-galactosidase fragments and resulting in the formation of a functional enzyme that converted substrate to detectable signal. Cell handling and assays were performed according to protocols specified in the PathHunter® assays kits (DiscoverX, Fremont, Calif.). Assays were performed in quadruplicate in white 384 well plates. End point luminescence data were plotted and fit to a 4 parameter logistic function to obtain IC₅₀ values. For antagonist assays, an IC₈₀ concentration of agonist (LPA) equal to 0.5 micromolar was used.

Beta-Arrestin Based Assays for LPA and S1P Receptor Antagonists and Agonists (Human and Species Orthologs) Using Transiently Transfected Cells

CMV promoter based DNA constructs expressing a fusion of the LPA/S1P GPCR of interest and ProLink™ tag were used to transfect EA Parental™ CHO cells (DiscoverX, Fremont, Calif.) using a FuGENE® transfection kit (Roche). Beta-Arrestin based assays were conducted 24-48 hrs post transfection using PathHunter® assay kits (DiscoverX, Fremont, Calif.). Agonist and antagonist assays were performed in quadruplicate in white 384 well plates. End point luminescence data were plotted and fit to a 4 parameter logistic function to obtain IC₅₀ values. For antagonist assays, an IC₈₀ concentration of agonist (LPA) equal to 0.5 micromolar was used.

cAMP Based Assays for Human LPA1R Antagonists and Agonists

A CHO cell line stably expressing the human LPA1R (DiscoverX Inc, Fremont, Calif.) was used according to manufacturer's protocol. HitHunter® assay kits (DiscoverX, Fremont, Calif.) were used to measure cAMP levels. HitHunter® cAMP assays are competitive immunoassays. Free cAMP from cell lysates competed for antibody binding against labeled cAMP (ED-cAMP conjugate). Unbound ED-cAMP was free to complement EA to form active enzyme, which subsequently hydrolyzed substrate to produce signal. A positive signal generated was directly proportional to the amount of free cAMP bound by the binding protein. Forskolin (15 micromolar) was used to elevate cAMP levels. Increased LPA (agonist) activity was measured as a decrease in cAMP levels. For antagonist assays, an IC₈₀ of LPA (agonist) equal to 50 micromolar was used, and increased antagonist activity of the test compound was recorded as an increase in cAMP levels. All assays were performed in quadruplicate in white 384 well plates. End point luminescence data were plotted and fit to a 4 parameter logistic function to obtain IC₅₀ values.

LPA1 Chemotaxis Assay

Chemotaxis of the A2058 human melanoma cells is measured using the Neuroprobe ChemoTx® System plates (8 μm pore size, 5.7 mm diameter sites). The filter sites are coated with 0.001% fibronectin (Sigma) in 20 mM Hepes, pH 7.4 and allowed to dry. A2058 cells are serum-starved for 24 hours, then are harvested with Cell Stripper and are resuspended in DMEM containing 0.1% fatty-acid-free bovine serum albumin (BSA) to a concentration of 1.times.10.sup.6/ml. Cells are mixed with an equal volume of test compound (2×) in DMEM containing 0.1% fatty-acid-free BSA and incubated at 37° C. for 15 minutes. LPA (100 nM in DMEM containing 0.1% fatty-acid-free BSA) or vehicle is added to each well of the lower chamber and 50 μl of the cell suspension/test compound mix is applied to the upper portion of the ChemoTx® plate. Plates are incubated at 37° C. for three hours and then the cells are removed from the upper portion by rinsing with PBS and scraping. The filter is dried then stained with HEMA 3 Staining System (Fisher Scientific). The absorbance of the filter is read at 590 nM and IC₅₀, are determined using Symyx Assay Explorer.

LPA1 Migration Assay

Migration of primary fibroblasts (including lung, dermal), HFL-1, 3T3 and CHO cells expressing LPA1R were monitored using the Oris™ assay (Platypus Technologies, Madison, Wis.). These cells were dye (Cell Tracker Green™) loaded and serum starved for 12-24 hrs. In response to chemoattractants such as LPA and serum, the cells migrated inward in to the exclusion (detection) zone. After fixing, fluorescent cells in the detection zone were counted using a high content reader. The ability of LPA1 antagonists to inhibit cell migration is quantified by plotting cell number vs. compound concentration and curve fitting the resulting dose-response curve to a 4 parameter logistic function.

Assay of Inhibitory Effect on Cell Proliferation ([³H] Thymidine Incorporation)

Fibroblasts (primary human lung and dermal, HFL-1, 3T3 etc) are plated on a 96-well plate and serum starved for 24-48 hours. The media are then exchanged for media containing stimulants (LPA, TGFb, serum etc) and cultured further for 16-24 hours before [³H] thymidine addition. After culturing for another 8 hours, cells are washed with PBS and the amount of [³H] thymidine incorporated into the cells are assayed by Betaplate filter counter system (Amersham Pharmacia Biotech). The difference between the amount of [³H] thymidine incorporated in the stimulant-added well and the amount of [³H] thymidine incorporated in the well containing no stimulant represents the amount of [³H] thymidine incorporation accelerated by stimulant. The increase of [³H] thymidine incorporation without the addition of test compounds is set as 100% and the concentration of compound with 50% inhibition in the increase of [³H] thymidine incorporation (IC₅₀ value) is determined. The test compounds are added 0-30 min before stimulant addition.

Assay of Inhibitory Effect on Cell Proliferation (BrdU Incorporation)

Fibroblasts (primary human lung and dermal, HFL-1, 3T3 etc) were plated on a 96-well plate and serum starved for 24-48 hours. The media were then exchanged for media containing stimulants (LPA, TGFb, serum etc) and cultured further for 16-24 hours before BrdU addition. After culturing for another 8 hours, cells were washed with PBS and the amount of BrdU incorporated into the cells was assayed by absorbance at 450 nm using the Cell proliferation ELISA system (RPN250, Amersham LIFE SCIENCE). The difference between the amount of BrdU incorporated in the stimulant-added well and the amount of BrdU incorporated in the well containing no stimulant represented the amount of BrdU incorporation accelerated by stimulant. The increase of BrdU incorporation without the addition of test compounds was set as 100% and the concentration of compound with 50% inhibition in the increase of BrdU incorporation (IC₅₀ value) was determined. The test compounds were added 0-30 min before stimulant addition.

Myofibroblast Differentiation

Fibroblasts (primary human lung and dermal, HFL-1, 3T3 etc) are plated on a 96-well plate and serum starved for 24-48 hours. The media are then exchanged for media containing stimulants (LPA, TGFb, etc) and cultured further for 24-48 hours. The amount of alpha smooth muscle actin (aSMA) is quantitated using an ELISA kit (Thermo Scientific, USA). Alternatively after fixing and permeabilization, aSMA is also quantitated using immunohistochemical methods (FITC conjugated anti-aSMA, Sigma).

Assay for Effect of Compounds on Collagen Production

HFL-1 Cells (ATCC, Rockville, Md.) are grown under regular tissue culture conditions in complete media containing 10% fetal bovine serum (FBS; Mediatech, Inc. Herndon, Va.). Cells in early passage are plated in 6 well plates. When the cells reach confluence, the media is removed, cells are washed with PBS, and the cells are kept overnight in complete media containing 0.1% FBS. The media is then replaced with fresh media plus 0.1% FCS, 10 flM L-Proline (EMD Chemicals, Gibbstown, N.J.), 20 μg/mL ascorbic acid (EMD Chemicals, Gibbstown, N.J.). Compounds are added to triplicate wells to a final concentration of 1 mM from 100× stock solutions in DMSO. One hour after the addition of compound, the cells are treated with TGFb (Sigma-Aldrich, St. Louis, Mo.) to a final concentration of 10 ng/mL (25 ng total). Three days after addition of TGFb the media is removed, cells are washed with PBS and then lysed. The total collagen content of lysed cells is assessed with a dye-based collagen assay (Sircol Collagen Assay, Newtownabbey, Northern Ireland) and an flQuant plate-based spectrophotometer (BioTek Instruments, Inc., Winooski, Vt.) with appropriate standard curves. The dynamic range of the assay is defined by cells that were mock treated (1% DMSO without compound) in the presence and absence of TGFb.

Bleomycin-Induced Lung Fibrosis Model in Mice or Rats

Female C57Bl/6CD-1 mice (Harlan, 25-30 g) or Wistar rats (Harlan, 200-250 g) are housed 4 per cage, given free access to food and water and allowed to acclimate for at least 7 days prior to test initiation. After the habituation phase, animals are lightly anesthetized with isoflurane (5% in 100% O₂) and administered with bleomycin sulfate (Henry Schein) via intratracheal instillation (Cuzzocrea S et al. Am J Physiol Lung Cell Mol. Physiol. 2007 May; 292(5):L1095-104. Epub 2007 Jan. 12.). Animals are returned to their cages and monitored daily for the duration of the experiment. Test compound or vehicle is delivered po, ip, or sc daily. The route and frequency of dosing is based on previously determined pharmacokinetic properties. All animals are sacrificed using inhaled isoflurane 3, 7, 14, 21 or 28 days after bleomycin instillation. Following sacrifice, animals are intubated with a 20 gauge angiocatheter attached to a 1 ml syringe. Lungs are lavaged with saline to obtain bronchoalveolar lavage fluid (BALF) and then removed and fixed in 10% neutral buffered formalin for subsequent histopathological analysis. BALF is centrifuged for 10 min at 800×g to pellet the cells and the cell supernatant removed and frozen at −80° C. for subsequent protein analysis using the DC protein assay kit (Biorad, Hercules, Calif.) and soluble collagen analysis using Sircol (Biocolor Ltd, UK). BALF is analyzed for concentrations of inflammatory, pro-fibrotic and tissue injury biomarkers including transforming growth factor β1, hyaluronic acid, tissue inhibitor of metalloproteinase-1, matrix matelloproteinase-7, connective tissue growth factor and lactate dehydrogenase activity, using commercially available ELISA. The cell pellet is re-suspended in PBS. Total cell counts are then obtained using a Hemavet hematology system (Drew Scientific, Wayne, Pa.) and differential cells counts are determined using Shandon cytospin (Thermo Scientific, Waltham, Mass.). Lung tissue is stained using hematoxylin and eosin (H&E) and trichrome and lung fibrosis is determined by semiquantitative histopathological scoring (Ashcroft T. et al. J. Clin. Path. 1988; 41; 4, 467-470) using light microscopy (10× magnification) and quantitative, computer-assisted densitometry of collagen in lung tissue sections using light microscopy. The data are plotted using Graphpad prism and statistical differences between groups determined.

Mouse Carbon Tetrachloride (CCl4)-Induced Liver Fibrosis Model

Female C57BL/6 mice (Harlan, 20-25 g) housed 4/cage are given free access to food and water and allowed to acclimate for at least 7 days prior to test initiation. After the habituation phase, mice receive CCl.sub.4 (0.5-1.0 ml/kg body weight) diluted in corn oil vehicle (100 μL volume) via i.p. injection twice a week for 84-6 weeks. (Higazi, A. A. et al., Clin Exp Immunol. 2008 April; 152(1):163-73. Epub 2008 Feb. 14.). Control mice receive an equivalent volume of corn oil vehicle only. Test compound or vehicle is delivered po, ip, or sc daily. At the end of the study (8 weeks after first i.p. injection of CCl₄), mice are sacrificed using inhaled isoflurane and blood is drawn via cardiac puncture for subsequent analysis of ALT/AST levels. The liver is harvested, and one half of the liver is frozen at −80° C. and the other half is fixed in 10% neutral buffered formalin for histological assessment of liver fibrosis using light microscopy (10× magnification). Liver tissue homogenates are analyzed for collagen levels using Sircol (Biocolor Ltd, UK). Fixed Liver tissue is stained using hematoxylin and eosin (H&E) and trichrome and liver fibrosis is determined by quantitative, computer-assisted densitometry of collagen in liver tissue sections using light microscopy. Plasma and liver tissue lysates are also analyzed for concentrations of inflammatory, pro-fibrotic and tissue injury biomarkers including transforming growth factor β1, hyaluronic acid, tissue inhibitor of metalloproteinase-1, matrix matelloproteinase-7, connective tissue growth factor, and lactate dehydrogenase activity, using commercially available ELISA. The resulting data are plotted using Graphpad prism and statistical differences between groups determined.

Mouse Intravenous LPA-Induced Histamine Release

A mouse intravenous LPA-induced histamine release model is utilized to determine the in vivo potency of LPA₁ and LPA₃ receptor antagonists. Female CD-1 mice (weighing 25-35 grams) are administered compound (i.p., s.c. or p.o.) in a volume of 10 ml/kg 30 minutes to 24 hours prior to intravenous LPA challenge (300 μg/mouse in 0.1% FAF BSA). Immediately following LPA challenge mice are placed into an enclosed Plexiglas chamber and exposed to an isoflurane for a period of 2-10 minutes. They are removed, and blood collected into tubes containing EDTA. Blood is then centrifuged at 10,000×g for 10 minutes at 4° C. Histamine concentrations in the plasma are determined by EIA. Drug concentrations in plasma are determined by mass spectrometry. The dose to achieve 50% inhibition of blood histamine release is calculated by nonlinear regression (Graphpad Prism) and plotted as the ED₅₀. The plasma concentration associated with this dose is plotted as the EC₅₀.

Mouse Unilateral Ureteral Obstruction Kidney Fibrosis Model

Female C57BL/6 mice (Harlan, 20-25 g) housed 4/cage will be given free access to food and water and allowed to acclimate for at least 7 days prior to test initiation. After the habituation phase, mice undergo unilateral ureteral obstruction (UUO) surgery or sham to left kidney. Briefly, a longitudinal, upper left incision is performed to expose the left kidney. The renal artery is located and 6/0 silk thread is passed between the artery and the ureter. The thread is looped around the ureter and knotted 3 times insuring full ligation of ureter. The kidney is returned to abdomen, the abdominal muscle is sutured and the skin is stapled closed. Mice are returned to their cages and monitored daily for the duration of the experiment. Test compound or vehicle is delivered po, ip, or sc daily. The route and frequency of dosing is based on previously determined pharmacokinetic properties. All animals are sacrificed using inhaled isoflurane 4, 8, 14, 21, or 28 days after UUO surgery. Following sacrifice blood is drawn via cardiac puncture, the kidneys are harvested and one half of the kidney is frozen at −80° C. and the other half is fixed in 10% neutral buffered formalin for histological assessment of kidney fibrosis using light microscopy (10× magnification). Kidney tissue homogenates are analyzed for collagen levels using Sircol (Biocolor Ltd, UK). Fixed kidney tissue is also stained using hematoxylin and eosin (H&E) and trichrome and kidney fibrosis is determined by quantitative, computer-assisted densitometry of collagen in liver tissue sections using light microscopy and collagen content in kidney lysate. Plasma and kidney tissue lysates are also analyzed for concentrations of inflammatory, pro-fibrotic and tissue injury biomarkers including transforming growth factor β1, hyaluronic acid, tissue inhibitor of metalloproteinase-1, matrix matelloproteinase-7, connective tissue growth factor and plasminogen activator inhibitor-1 lactate dehydrogenase activity, using commercially available ELISA. The resulting data are plotted using Graphpad prism and statistical differences between groups determined.

Mouse Dermal Vascular Leak Assay

Female BALB/c mice (Harlan) weighing 20-25 grams are given free access to standard mouse chow and water and are allowed to acclimate for two weeks prior to study initiation. Compounds are prepared in at a range of concentrations and delivered by oral gavage. Three hours following dose, mice are placed into a restraining device and given Evan's blue dye intravenously by tail vein injection (0.2 ml of a 0.5% solution). Mice are then anesthetized using 3% isoflurane anesthesia to allow for intradermal injection of LPA (30 μg in 20 μll 0.1% fatty acid free BSA). Thirty minutes after LPA injection mice are sacrificed by CO₂ inhalation and the skin is removed from the challenge site and placed into 2 ml formamide for overnight extraction of Evan's blue dye. Following extraction, a 150 μl aliquot of formamide for each tissue sample is placed into a 96 well plate and read at 610 nm using a photospectrometer. The resulting data (OD units) are plotted using GraphPad Prizm.

Bleomycin Dermal Fibrosis Model

Bleomycin is dissolved in phosphate buffered saline (PBS) at 10 ug/ml, and sterilized by filtration. Bleomycin or PBS control (100 ul) is injected subcutaneously into two locations on the shaved back of C57/BL6 or S 129 mice (Charles River/Harlan Labs, 20-25 g) once daily for 28 days while under isoflourane anesthesia (5% in 100% O₂). Test compounds or controls are administered throughout the study via subcutaneous or intraperitoneal injection, or via oral gavage. After 28 days, mice are euthanized and 6 mm-full thickness punch biopsies are obtained from each injection site. Dermal fibrosis is assessed by histopathology and hydroxyproline biochemical assays.

Rat Dermal Wound Healing

Female rats (Harlan Labs, 200-250 g) are given a single 1 cm-full thickness incisional wound on the back while under isoflourane anesthesia. The incision is placed parallel to the midline along the dorsal skin, using a surgical scalpel. For excisional wounds, an 8 mm-full thickness skin biopsy punch is made on the back of each animal opposite to the site of the incision. Test compounds are administered prior to wounding, and dosed for 14 days. Wounds are allowed to heal, and photographs are taken and analyzed digitally to measure wound healing throughout the study. At the end of the study animals are euthanized and wound closure determined.

### Assay Data for Compounds

Compounds of the preferred embodiments were prepared according to the methods described herein and assay data obtained for Beta Arrestin EC₅₀ assay, cell migration EC₅₀ assay, and Ca Flux LPA1 IC₅₀ assay. Control compounds were also prepared and assay data obtained. The assay data obtained for Beta Arrestin EC₅₀ assay, cell migration EC₅₀ assay and Ca Flux LPA1 IC₅₀ assay are presented in Table 13, 14 and 15 respectively, in which A=greater than 500 nM, B=greater than or equal to 50 nM and less than or equal to 500 nM; and C=less than 50 nM.

TABLE 13

| Compd. | Beta Arrestin EC₅₀ |
|---|---|
| IT001 | B |
| IT002 | B |
| IT003 | C |
| IT004 | C |
| IT005 | A |
| IT006 | B |
| IT007 | A |
| IT008 | A |
| IT009 | A |
| IT010 | A |
| IT011 | B |
| IT012 | A |
| IT013 | A |
| IT014 | C |
| IT015 | C |
| IT016 | C |
| IT017 | C |
| IT018 | A |
| IT019 | A |
| IT020 | A |
| IT021 | A |
| IT022 | A |
| IT023 | A |
| IT024 | A |
| IT025 | A |
| IT026 | A |
| IT027 | B |
| IT028 | C |
| IT029 | C |
| IT030 | A |
| IT031 | C |
| IT032 | C |
| IT033 | C |
| IT034 | C |
| IT035 | C |
| IT036 | A |
| IT037 | B |
| IT038 | B |
| IT039 | B |
| IT040 | A |
| IT041 | A |
| IT042 | C |
| IT043 | C |
| IT044 | C |
| IT045 | A |
| IT046 | C |
| IT047 | C |
| IT048 | C |
| IT049 | C |
| IT050 | C |
| IT051 | B |
| IT053 | A |
| IT054 | A |
| IT055 | B |
| IT056 | C |
| IT057 | A |
| IT058 | A |
| IT059 | B |
| IT060 | A |
| IT061 | C |
| IT062 | A |
| IT063 | A |
| IT064 | C |
| IT065 | B |
| IT066 | B |
| IT067 | B |
| IT068 | A |
| IT069 | C |
| IT070 | C |
| IT071 | C |
| IT072 | C |
| IT073 | B |
| IT074 | B |
| IT075 | C |
| IT076 | A |
| IT077 | B |
| IT078 | C |
| IT079 | A |
| IT080 | A |
| IT081 | A |
| IT082 | B |
| IT083 | A |
| IT084 | C |
| IT085 | C |
| IT086 | B |
| IT087 | C |
| IT088 | C |
| IT089 | A |
| IT090 | C |
| IT091 | B |
| IT092 | A |
| IT093 | A |
| IT094 | C |
| IT095 | C |
| IT096 | C |
| IT097 | B |
| IT098 | C |
| IT099 | B |
| IT 100 | C |
| IT101 | A |
| IT 102 | C |
| IT 103 | C |

TABLE 14

| Compd. | Cell Migration EC₅₀ |
|---|---|
| IT001 | B |
| IT002 | B |
| IT003 | B |
| IT004 | C |
| IT005 | A |
| IT006 | B |
| IT007 | A |
| IT008 | A |
| IT009 | A |
| IT010 | A |
| IT011 | A |
| IT012 | A |
| IT013 | A |
| IT014 | B |
| IT015 | B |
| IT016 | B |
| IT017 | C |
| IT018 | A |
| IT019 | A |
| IT020 | A |
| IT021 | A |
| IT022 | A |
| IT023 | A |
| IT024 | A |
| IT025 | A |
| IT026 | A |
| IT027 | A |
| IT028 | C |
| IT029 | B |
| IT030 | A |
| IT031 | B |
| IT032 | B |
| IT033 | C |
| IT034 | B |
| IT035 | B |
| IT036 | A |
| IT038 | A |
| IT039 | A |
| IT040 | A |
| IT041 | A |
| IT042 | B |
| IT043 | B |
| IT044 | B |
| IT045 | A |
| IT046 | B |
| IT047 | C |
| IT048 | B |
| IT049 | B |
| IT050 | B |
| IT051 | B |
| IT052 | A |
| IT053 | A |
| IT054 | A |
| IT055 | B |
| IT056 | B |
| IT057 | A |
| IT058 | A |
| IT059 | A |
| IT060 | A |
| IT061 | B |
| IT062 | A |
| IT063 | A |
| IT064 | B |
| IT065 | A |
| IT066 | A |
| IT067 | A |
| IT068 | A |
| IT069 | B |
| IT070 | C |
| IT071 | B |
| IT072 | A |
| IT073 | B |
| IT074 | A |
| IT075 | B |
| IT078 | B |
| IT079 | A |
| IT081 | A |
| IT082 | B |
| IT083 | A |
| IT084 | B |
| IT085 | A |
| IT086 | A |
| IT087 | C |
| IT088 | B |
| IT089 | A |
| IT090 | B |
| IT091 | A |
| IT092 | A |
| IT093 | A |
| IT094 | B |
| IT095 | C |
| IT096 | B |
| IT097 | A |
| IT098 | C |
| IT099 | A |

TABLE 15

| Compd. | Ca Flux LPA1 IC₅₀ |
|---|---|
| IT003 | C |
| IT004 | C |
| IT009 | A |
| IT010 | A |
| IT014 | C |
| IT015 | B |
| IT016 | C |
| IT017 | C |
| IT018 | A |
| IT020 | A |
| IT021 | A |
| IT022 | A |
| IT023 | A |
| IT026 | A |
| IT027 | B |
| IT028 | C |
| IT029 | B |
| IT030 | A |
| IT031 | B |
| IT032 | B |
| IT033 | B |
| IT034 | B |
| IT035 | A |
| IT040 | A |
| IT041 | B |
| IT043 | B |
| IT046 | A |
| IT047 | B |
| IT048 | C |
| IT050 | B |
| IT051 | B |
| IT056 | B |
| IT061 | C |
| IT064 | C |
| IT066 | B |
| IT067 | B |
| IT069 | C |
| IT070 | C |
| IT071 | B |
| IT073 | C |
| IT099 | A |

### Clinical Trials in Humans

Clinical trials can be run in multiple conditions. The details of these trials differ based on the indication. Examples of clinical trials for assessment of clinical effect in idiopathic pulmonary fibrosis are provided below.

Although a duration of 72 weeks is specified in the examples below, other durations can also be employed, e.g., 52 weeks.

Clinical Trial in Humans with Idiopathic Pulmonary Fibrosis (IPF) Purpose—Example #1

The efficacy of treatment with a compound of a preferred embodiment compared with placebo in patients with idiopathic pulmonary fibrosis (IPF) and the safety of treatment with a compound of a preferred embodiments compared with placebo in patients with IPF is assessed.

The primary outcome variable is the absolute change in percent predicted forced vital capacity (FVC) from baseline to Week 72. Other possible end-points would include, but are not limited to: mortality, progression free survival, change in rate of FVC decline, change in SpO2, and change in biomarkers (HRCT image analysis; molecular and cellular markers of disease activity). Secondary outcome measures include: composite outcomes of important IPF-related events; progression-free survival; categorical assessment of absolute change in percent predicted FVC from baseline to Week 72; change in Shortness-of-Breath from baseline to Week 72; change in percent predicted hemoglobin (Hb)-corrected carbon monoxide diffusing capacity (DLco) of the lungs from baseline to Week 72; change in oxygen saturation during the 6 minute walk test (6MWT) from baseline to Week 72; change in high-resolution computed tomography (HRCT) assessment from baseline to Week 72; change in distance walked in the 6MWT from baseline to Week 72.

Patients eligible for this study include, but are not limited to: those patients that satisfy the following inclusion criteria: diagnosis of IPF; 40 to 80 years of age; FVC≧50% predicted value; DLco≧35% predicted value; either FVC or DLco≧90% predicted value; no improvement in past year; able to walk 150 meters in 6 minutes and maintain saturation≧83% while on no more than 6 L/min supplemental oxygen.

Patients are excluded from this study if they satisfy any of the following criteria: unable to undergo pulmonary function testing; evidence of significant obstructive lung disease or airway hyper-responsiveness; in the clinical opinion of the investigator, the patient is expected to need and be eligible for a lung transplant within 72 weeks of randomization; active infection; liver disease; cancer or other medical condition likely to result in death within 2 years; diabetes; pregnancy or lactation; substance abuse; personal or family history of long QT syndrome; other IPF treatment; unable to take study medication; withdrawal from other IPF trials.

Patients are orally dosed with either placebo or an amount of a compound of a preferred embodiment (1 mg/day-1000 mg/day). The primary outcome variable will be the absolute change in percent predicted FVC from Baseline to Week 72. Patients will receive blinded study treatment from the time of randomization until the last patient randomized has been treated for 72 weeks. A Data Monitoring Committee (DMC) will periodically review safety and efficacy data to ensure patient safety.

After week 72, patients who meet the Progression of Disease (POD) definition, which is a ≧10% absolute decrease in percent predicted FVC or a ≧15% absolute decrease in percent predicted DLco, will be eligible to receive permitted IPF therapies in addition to their blinded study drug. Permitted IPF therapies include, but are not limited to: corticosteroids, azathioprine, cyclophosphamide, and N-acetyl-cysteine.

In a preferred aspect, a method is provided of administering an LPA1 antagonist of a preferred embodiment to a patient with pulmonary fibrosis (e.g., a patient with IPF), wherein said patient is selected, or diagnosed, or identified to have one or more of the following criteria: (1) ratio of forced expiratory volume in one second (FEV1) to forced vital capacity volume (FVC), or FEV1/FVC, is greater than 0.80, (2) percent of predicted FVC (% FVC) is 90% or less, for example ranging from 50% to 90%, inclusive of both endpoints, and (3) time since diagnosis of IPF is at least six months and up to 48 months. The terms “selecting,” “diagnosing” and “identifying” are used synonymously with respect to a patient.

Clinical Trial in Humans with Idiopathic Pulmonary Fibrosis (IPF) Purpose—Example #2

The efficacy of treatment with a compound of a preferred embodiment compared with placebo in patients with idiopathic pulmonary fibrosis (IPF) and the safety of treatment with a compound of a preferred embodiments compared with placebo in patients with IPF is assessed.

The primary outcome variable includes, but is not limited to, the absolute change in percent predicted forced vital capacity (FVC) from baseline to Week 72. Secondary outcome measures include, but are not limited to: composite outcomes of important IPF-related events; progression-free survival; categorical assessment of absolute change in percent predicted FVC from baseline to Week 72; change in Shortness-of-Breath from baseline to Week 72; change in percent predicted hemoglobin (Hb)-corrected carbon monoxide diffusing capacity (DLco) of the lungs from baseline to Week 72; change in oxygen saturation during the 6 minute walk test (6MWT) from baseline to Week 72; change in high-resolution computed tomography (HRCT) assessment from baseline to Week 72; change in distance walked in the 6MWT from baseline to Week 72.

Patients eligible for this study include, but are not limited to, those patients that satisfy the following inclusion criteria: diagnosis of IPF; 40 to 80 years of age; FVC≧50% predicted value; DLco≧35% predicted value; either FVC or DLco≧90% predicted value; no improvement in past year; able to walk 150 meters in 6 minutes and maintain saturation≧83% while on no more than 6 L/min supplemental oxygen.

Patients are excluded from this study if they satisfy any of the following criteria, including but not limited to: unable to undergo pulmonary function testing; evidence of significant obstructive lung disease or airway hyper-responsiveness; in the clinical opinion of the investigator, the patient is expected to need and be eligible for a lung transplant within 72 weeks of randomization; active infection; liver disease; cancer or other medical condition likely to result in death within 2 years; diabetes; pregnancy or lactation; substance abuse; personal or family history of long QT syndrome; other IPF treatment; unable to take study medication; withdrawal from other IPF trials.

Patients are orally dosed with either placebo or an amount of a compound of a preferred embodiment (1 mg/day-1000 mg/day or more). The primary outcome variable includes, but is not limited to, the absolute change in percent predicted FVC from Baseline to Week 72. Patients receive blinded study treatment from the time of randomization until the last patient randomized has been treated for 72 weeks. A Data Monitoring Committee (DMC) periodically reviews safety and efficacy data to ensure patient safety.

After week 72, patients who meet the Progression of Disease (POD) definition, which is a ≧10% absolute decrease in percent predicted FVC or a ≧15% absolute decrease in percent predicted DLco, are eligible to receive permitted IPF therapies in addition to their blinded study drug. Permitted IPF therapies include, but are not limited to, corticosteroids, azathioprine, cyclophosphamide, and N-acetyl-cysteine.

### Treatment of Ideopathic Pulmonary Fibrosis

A compound of a preferred embodiment can be administered to a patient in need of therapy, and can be used in methods of preparing or packaging medicaments, containers, packages, and kits comprising the compound of a preferred embodiment. The patient may have pulmonary fibrosis, such as IPF, and the medicament can be used for treatment of pulmonary fibrosis, or IPF. A selected group of IPF patients that are more likely to experience FVC decline and disease progression over a period of a year can be identified and treated. Their greater rate of progression, as reflected by a greater rate of decrease in respiratory parameters such as FVC, correlates with a greater relative magnitude of treatment effect. In certain embodiments, IPF patients with the following criteria experience a greater FVC decline, as measured by % FVC change from baseline or proportion of patients with 10% or greater % FVC decline at a specified timepoint, compared to patients that do not meet the criteria. Patients with the following criteria also exhibit a greater observed treatment effect on alleviating the extent of FVC decline compared to patients that do not meet the criteria: (a) % FVC 50%-90%; (b) FEV1/FVC ratio >0.80; (c) Time since IPF diagnosis>0.5 years and <48 months;

A method of treating pulmonary fibrosis, optionally IPF, is provided comprising (a) selecting a patient that exhibits (i) percent of predicted forced vital capacity volume (% FVC) of about 90% or less, or (ii) ratio of forced expiratory volume in one second (FEV1) to forced vital capacity volume (FVC) of about 0.80 or greater, or both, and (b) administering a therapeutically effective amount of the compound of a preferred embodiment.

In a related aspect, use is provided of the compound of a preferred embodiment in treating pulmonary fibrosis in a patient that exhibits (i) percent of predicted forced vital capacity volume (% FVC) of about 90% or less or (ii) ratio of forced expiratory volume in one second (FEV1) to forced vital capacity volume (FVC) of about 0.80 or greater, or both.

In a further related aspect, the compound of a preferred embodiment is used in preparation of a medicament for treating pulmonary fibrosis in a patient that exhibits (i) percent of predicted forced vital capacity volume (% FVC) of about 90% or less or (ii) ratio of forced expiratory volume in one second (FEY1) to forced vital capacity volume (FVC) of about 0.80 or greater, or both.

Optionally, in some or any of these embodiments, % FVC ranges from about 50% to about 90%. In some or any embodiments, the patient has been diagnosed with pulmonary fibrosis, optionally IPF, for at least six months, and optionally less than 48 months. In some or any embodiments, optionally the patient is also selected to exhibit a percent of diffusing capacity (% DL_{co}) of about 90% or less, for example, ranging from 30% to 90%, or 30% to 60%, inclusive of both endpoints. In some or any embodiments, the FEV1/FVC ratio is greater than 0.9. In some or any embodiments, the % FVC is less than 80%, 70%, or 60%. In some or any embodiments, the % DL_{co} is less than 90%, 80%, 70%, 60%, or 50%, or less than 40%. In most cases the patient is diagnosed with IPF through a High Resolution Computed Tomography (HRCT) scan, optionally with confirmation through surgical lung biopsy.

In any of the aspects or embodiments, the therapeutically effective amount of the compound of a preferred embodiment being administered may be a total daily dosage of from 1-4000 mg per day or more, e.g., at least about 1800 mg per day, or about 2400 mg or about 2403 mg per day, optionally administered in divided doses three times per day, with food. In any of the aspects of embodiments, the total daily dosage may be about 1200 to about 4000 mg per day, or about 1600 to about 3600 mg per day. In any of the aspects of the invention, the daily dosage may be administered in divided doses three times a day, or two times a day, or alternatively is administered in a single dose once a day. In any of the aspects of the invention, the compound of a preferred embodiment may be administered with food. For example, the daily dosage of 2400 mg or 2403 mg the compound of a preferred embodiment per day may be administered as follows: 801 mg taken three times a day, with food.

The compound of a preferred embodiment can be dosed at a total amount of from 1-4000 mg per day or more, or from about 50 to about 2400 mg per day. The dosage can be divided into two or three doses over the day. Specific amounts of the total daily amount of the therapeutic contemplated for the disclosed methods include about 50 mg, about 100 mg, about 150 mg, about 200 mg, about 250 mg, about 267 mg, about 300 mg, about 350 mg, about 400 mg, about 450 mg, about 500 mg, about 534 mg, about 550 mg, about 600 mg, about 650 mg, about 700 mg, about 750 mg, about 800 mg, about 850 mg, about 900 mg, about 950 mg, about 1000 mg, about 1050 mg, about 1068 mg, about 1100 mg, about 1150 mg, about 1200 mg, about 1250 mg, about 1300 mg, about 1335 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1869 mg, about 1900 mg, about 1950 mg, about 2000 mg, about 2050 mg, about 2100 mg, about 2136 mg, about 2150 mg, about 2200 mg, about 2250 mg, about 2300 mg, about 2350 mg, and about 2400 mg.

Dosages of the compound of preferred embodiments can alternately be administered as a dose measured in mg/kg. Contemplated mg/kg doses of the disclosed therapeutics include, e.g., about 1 mg/kg to about 40 mg/kg. Specific ranges of doses in mg/kg include about 20 mg/kg to about 40 mg/kg, or about 30 mg/kg to about 40 mg/kg.

In another aspect, a package or kit is provided comprising the compound of a preferred embodiment, optionally in a container, and a package insert, package label, instructions, or other labeling including any of the criteria for patient selection described herein. The package insert, package label, instructions or other labeling may further comprise directions for treating IPF by administering the compound of a preferred embodiment, e.g., at a dosage of at least about 1800 mg per day, or a dosage of about 2400 mg or about 2403 mg per day.

In related aspect, a method of preparing or packaging a medicament comprising the compound of a preferred embodiment, optionally in a container, together with a package insert or package label or instructions including any of the foregoing information or recommendations.

In some embodiments, a method of treating IPF is disclosed comprising providing, selling, or delivering any of the kits of disclosed herein to a hospital, physician, or patient.

The following patent publications include disclosures relating to diseases, disorders, or conditions that may be associated with one or more of the lysophosphatidic acid receptors, the contents of which relating to said diseases, disorders, or conditions are hereby incorporated by reference herein: PCT Intl. Publ. No. WO/2011017350-A1; PCT Intl. Publ. No. WO/2010141768-A1; PCT Intl. Publ. No. WO/2010077883-A1; PCT Intl. Publ. No. WO/2010077882-A1; PCT Intl. Publ. No. WO/2010068775-A1; U.S. Pat. Publ. No. 20110098352-A1; U.S. Pat. Publ. No. 20110098302-A1; U.S. Pat. Publ. No. 20110082181-A1; U.S. Pat. Publ. No. 20110082164-A1; U.S. Pat. Publ. No. 20100311799-A1; U.S. Pat. Publ. No. 20100152257-A1; PCT Intl. Publ. No. WO/2010141761-A1; PCT Intl. Publ. No. WO/2011041729-A1; PCT Intl. Publ. No. WO/2011041694-A1; PCT Intl. Publ. No. WO/2011041462-A1; and PCT Intl. Publ. No. WO/2011041461-A1.

### Pharmaceutical Compositions

Parenteral Pharmaceutical Composition

To prepare a parenteral pharmaceutical composition suitable for administration by injection (subcutaneous, intravenous, or the like), 100 mg of a water-soluble salt/soluble material itself/solubilized complex of a compound of a preferred embodiment is dissolved in sterile water and then mixed with 10 mL of 0.9% sterile saline. The mixture is incorporated into a dosage unit form suitable for administration by injection.

Injectable Pharmaceutical Composition

To prepare an injectable formulation, 1.2 g of a compound of Formulas (I), 2.0 mL of sodium acetate buffer solution (0.4 M), HCl (1 N) or NaOH (1 M) (q.s. to suitable pH), water (distilled, sterile) (q.s. to 20 mL) are mixed. All of the above ingredients, except water, are combined and stirred and if necessary, with slight heating if necessary. A sufficient quantity of water is then added.

Oral Pharmaceutical Composition

To prepare a pharmaceutical composition for oral delivery, 100 mg of a compound of a preferred embodiment is mixed with 750 mg of starch. The mixture is incorporated into an oral dosage unit, such as a hard gelatin capsule, or 100 mg of compound is granulated with binder solution such as starch solution along with suitable diluents such as microcrystalline cellulose or like, disintegrants such as cross caramellose sodium, dry the resultant mixture and add lubricant and compress into tablet which is suitable for oral administration.

Sublingual (Hard Lozenge) Pharmaceutical Composition

To prepare a pharmaceutical composition for buccal delivery, such as a hard lozenge, 100 mg of a compound of a preferred embodiment is mixed with 420 mg of powdered sugar/mannitol/xylitol or such sugars that provide negative heat of solution to the system, 1.6 mL of light corn syrup, 2.4 mL distilled water, and 0.42 mL mint extract or other flavorants. The mixture is blended and poured into a mold to form a lozenge suitable for buccal administration.

Fast-Disintegrating Sublingual Tablet

A fast-disintegrating sublingual tablet is prepared by mixing 48.5% by weigh of a compound of a preferred embodiment, 20% by weight of microcrystalline cellulose (KG-802), 24.5% by weight of either mannitol or modified dextrose or combination that help dissolve the compressed tablet faster in the mouth, 5% by weight of low-substituted hydroxypropyl cellulose (50 μm), and 2% by weight of magnesium stearate. Tablets are prepared by direct compression (AAPS PharmSciTech. 2006; 7(2):E41). The total weight of the compressed tablets is maintained at 150 mg. The formulation is prepared by mixing the amount of the compound of a preferred embodiment with the total quantity of microcrystalline cellulose (MCC) and mannitol/modified dextrose or combination, and two-thirds of the quantity of low-substituted hydroxypropyl cellulose (L-HPC) by using a three dimensional manual mixer (Inversina®, Bioengineering AG, Switzerland) for 4.5 minutes. All of the magnesium stearate (MS) and the remaining one-third of the quantity of L-HPC are added 30 seconds before the end of mixing.

Inhalation Pharmaceutical Composition

To prepare a pharmaceutical composition for inhalation delivery, 20 mg of a compound of a preferred embodiment is mixed with 50 mg of anhydrous citric acid and 100 mL of 0.9% sodium chloride solution. The mixture is incorporated into an inhalation delivery unit, such as a nebulizer, which is suitable for inhalation administration.

Nebulizer Suspension Pharmaceutical Composition

In another embodiment, a compound of a preferred embodiment (500 mg) is suspended in sterile water (100 mL); Span 85 (1 g) is added followed by addition of dextrose (5.5 g) and ascorbic acid (10 mg). Benzalkonium chloride (3 mL of a 1:750 aqueous solution) is added and the pH is adjusted to 7 with phosphate buffer. The suspension is packaged in sterile nebulizers.

Rectal Gel Pharmaceutical Composition

To prepare a pharmaceutical composition for rectal delivery, 100 mg of a compound of a preferred embodiment is mixed with 2.5 g of methylcellulose (1500 mPa), 100 mg of methylparaben, 5 g of glycerin and 100 mL, of purified water. The resulting gel mixture is then incorporated into rectal delivery units, such as syringes, which are suitable for rectal administration.

Topical Gel Pharmaceutical Composition

To prepare a pharmaceutical topical gel composition, 100 mg of a compound of a preferred embodiment is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

Ophthalmic Solution

To prepare a pharmaceutical ophthalmic solution composition, 100 mg of a compound of a preferred embodiment is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

Nasal Spray Solution

To prepare a pharmaceutical nasal spray solution, 10 g of a compound of a preferred embodiment is mixed with 30 mL of a 0.05M phosphate buffer solution (pH 4.4). The solution is placed in a nasal administrator designed to deliver 100 μl of spray for each application.

While the disclosure has been illustrated and described in detail in the foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1.-69. (canceled)

70. A compound of Formula (III): 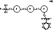
or a pharmaceutically acceptable salt thereof, wherein:
A is selected from 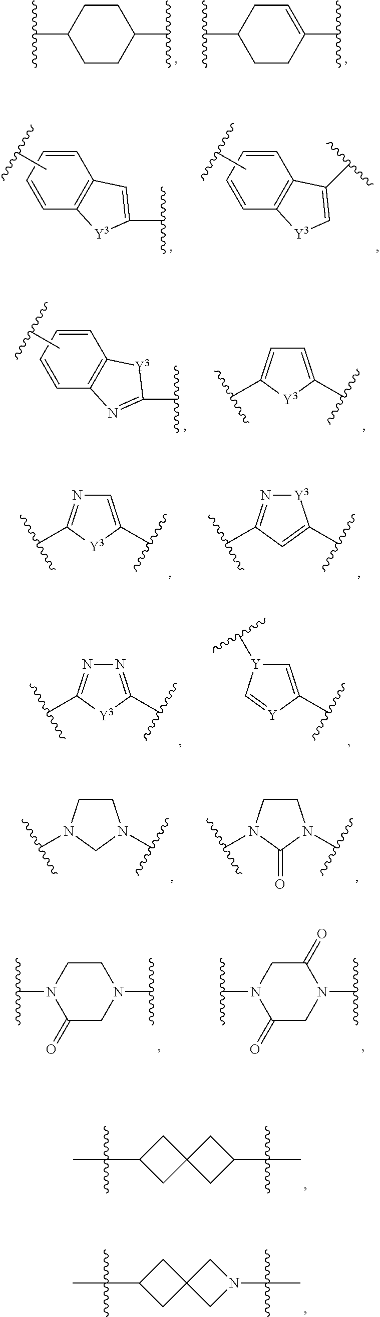 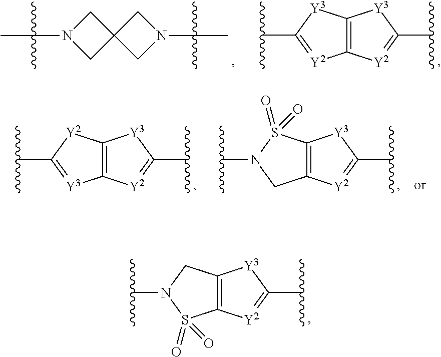
wherein A is unsubstituted or substituted with one or more substituents selected from alkyl, halogen, haloalkyl, cyano, hydroxy, alkoxy, haloalkoxy, or oxo; and
B is a ring system selected from the group consisting of 6-11 membered aryl, 5-11 membered heteroaryl, 4-11 membered heterocyclyl, and 4-11 membered carbocyclyl, and wherein B is unsubstituted or substituted with one or more substituents selected from alkyl, halogen, hydroxy, alkoxy, haloalkoxy, cyano, or oxo;
or alternatively,
B is selected from 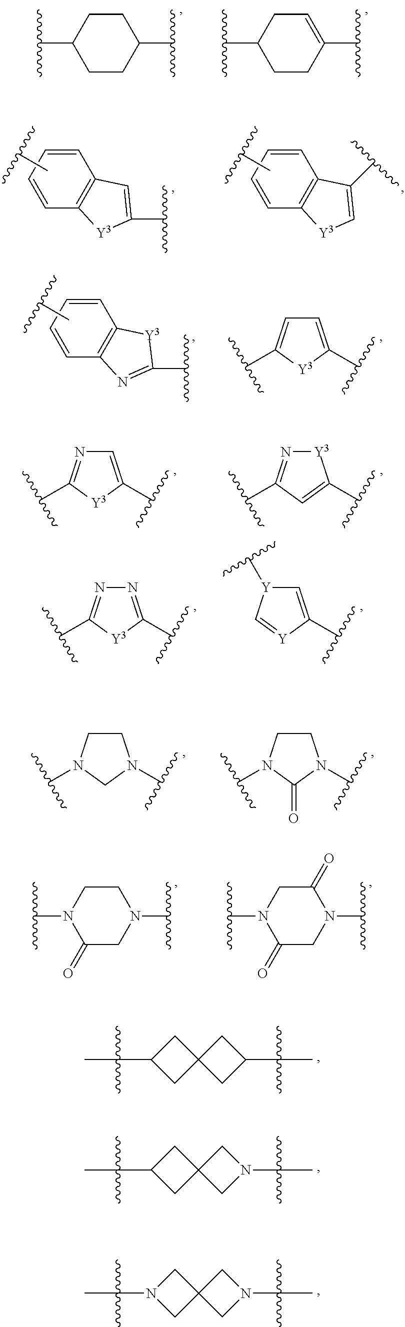 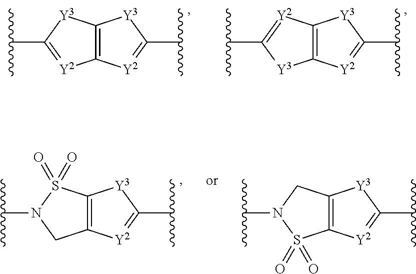
and A is a ring system selected from the group consisting of 6-11 membered aryl, 5-11 membered heteroaryl, 4-11 membered heterocyclyl, and 4-11 membered carbocyclyl, and wherein A is unsubstituted or substituted with one or more substituents selected from alkyl, halogen, haloalkyl, hydroxy, alkoxy, haloalkoxy, cyano, or oxo;
C is a ring system selected from the group consisting of 6-11 membered aryl, 5-11 membered heteroaryl, 5-11 membered heterocyclyl, and 5-11 membered carbocyclyl, wherein C is optionally substituted;
D is selected from —OH, 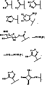
or carboxylic acid isosteres;
L⁴ is 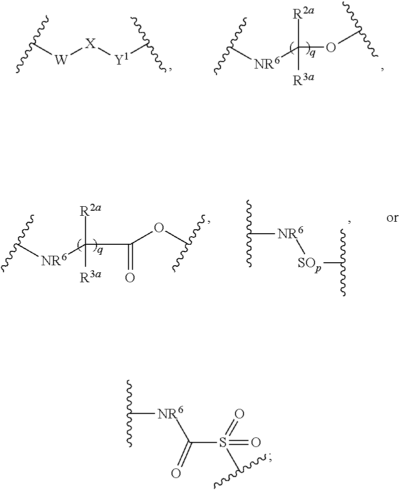
or alternatively, 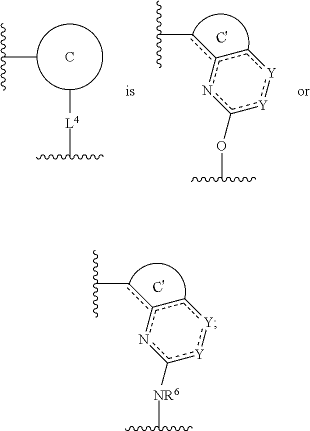
wherein 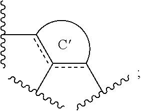
is selected from: 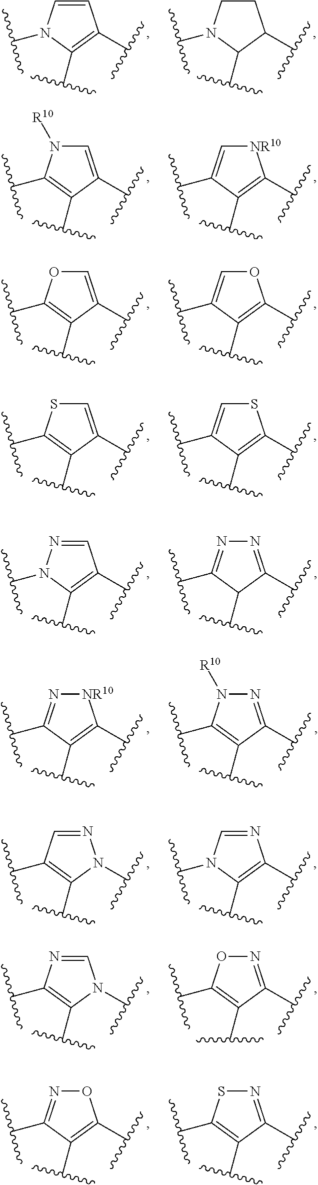 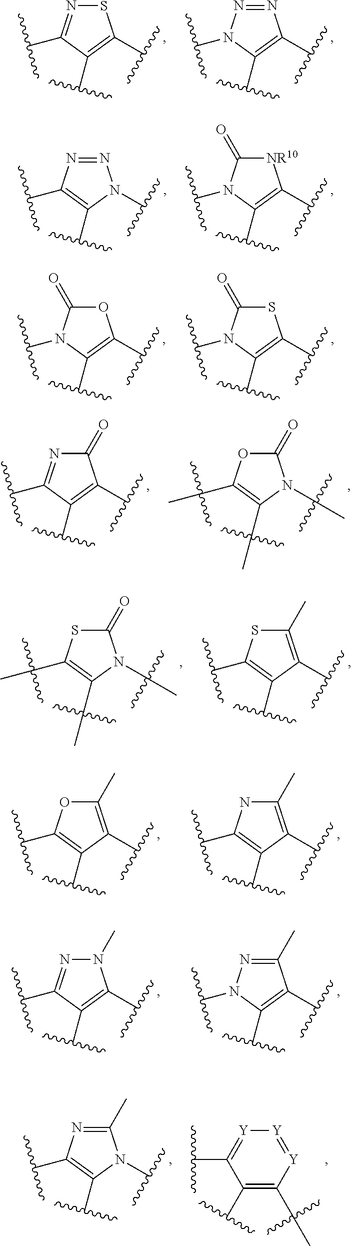
or optionally substituted variants thereof;
L¹ is selected from a single bond, a —O— linker, a —C(O)— linker, a —CH₂O— linker, a 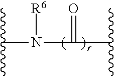
linker, a —C≡C— linker, or a —CH═CH— linker;
L² is selected from a single bond, a —O— linker, a 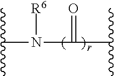
linker, a —C(O)— linker, a —CH₂— linker, a —CH₂O— linker, a —C≡C— linker, or a —CH═CH— linker;
L⁵ is selected from a single bond, a —CH₂O— linker, a —CH═CH— linker, a —C≡C—linker, 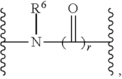
or a 4-7 membered heterocyclyl;
W is selected from C(R⁶)₂, NR⁶, or O;
X is selected from —C(O) or S(O)ₚ;
each Y is independently selected from CR⁶ or N;
Y¹ is selected from C(R⁶)₂, NR⁶, or O;
Y² is selected from —CH═ or N;
Y³ is selected from C(R⁶)₂, NR⁶, O, or S;
each Y⁴ is independently absent, CR⁹, C(R⁹)₂, N, or NH, provided that only one Y⁴ can be absent;
R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;
R² and R³ are each independently selected from hydrogen, alkyl, aryl, or heteroaryl; or R² and R³ are joined together with the atom to which they are attached to form an optionally substituted cycloalkyl or an optionally substituted heterocyclyl;
or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;
each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;
each R⁶ is independently selected from hydrogen; alkyl optionally substituted with one or more substituents selected from the group consisting of halogen, hydroxy and alkoxy; halogen; aryl; or C₃₋₆ cycloalkyl;
each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;
each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;
each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;
each R¹³ is independently selected from hydrogen, alkyl, haloalkyl, aryl, or C₃₋₆ cycloalkyl;
each R¹⁴ is independently selected from hydrogen, alkyl, haloalkyl, aryl, or C₃₋₆ cycloalkyl;
each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;
m is independently an integer from 0-3;
n is an integer from 0-3;
p is an integer from 1-2;
q is an integer from 1-6;
r is an integer of 0 or 1, and [symbol]
represents a single or double bond; provided that
when D is —C(O)OR¹; R¹ is hydrogen or alkyl; m is 1; A is cyclohexyl; B is phenyl; L³ is absent; L⁵ is a single bond; L¹ is a single bond; 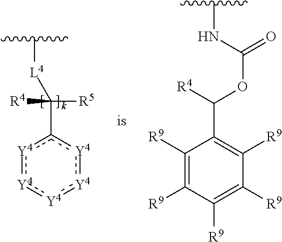
wherein R⁹ is selected from H, alkyl or halogen; then C cannot be a triazole or pyrazole;
when D is —C(O)OR¹; R¹ is hydrogen or alkyl; A is cyclohexyl or 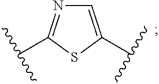 [SMILES: *c1cnc(*)s1]
B is phenyl; L³ is absent; L⁵ is a single bond; L¹ is a single bond; 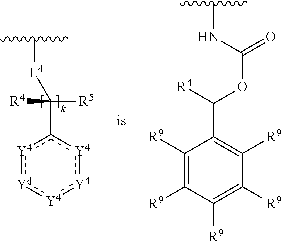
wherein R⁹ is selected from H, alkyl or halogen; and C is isoxazole; then m is not 0; and
when D is —C(O)OR¹; R¹ is hydrogen or alkyl; m is 1; A is phenyl; B is 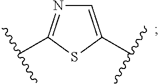 [SMILES: *c1cnc(*)s1]
L³ is absent; L⁵ is a single bond; L¹ is a single bond; 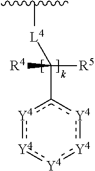
is 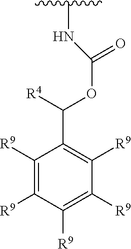
wherein R⁹ is selected from H, alkyl or halogen; then C is not isoxazole.

71. The compound or pharmaceutically acceptable salt thereof of claim 70, wherein
A is selected from 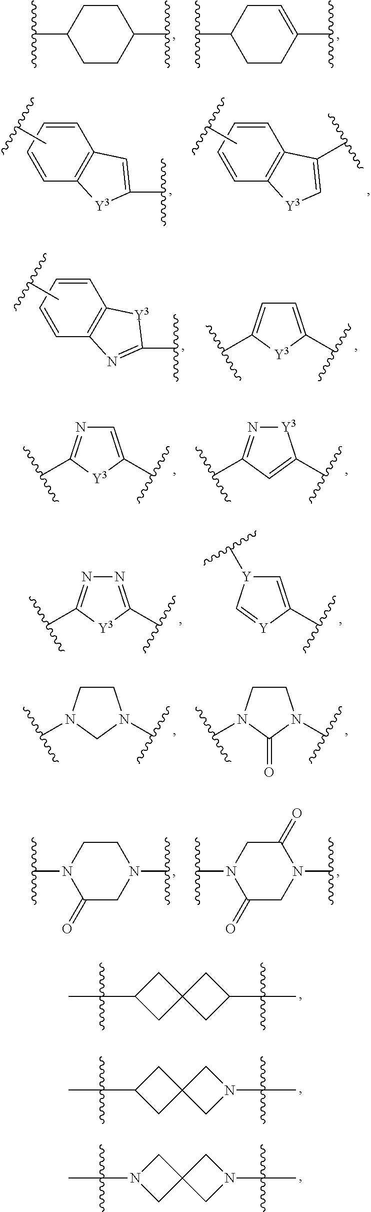 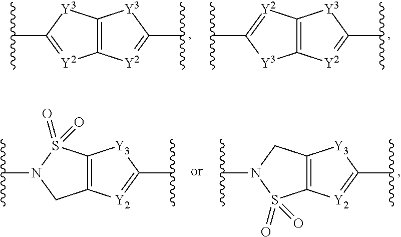
wherein A is unsubstituted or substituted with one or more substituents selected from alkyl, haloalkyl, halogen, hydroxy, alkoxy, haloalkoxy, cyano, or oxo; and
B is selected from 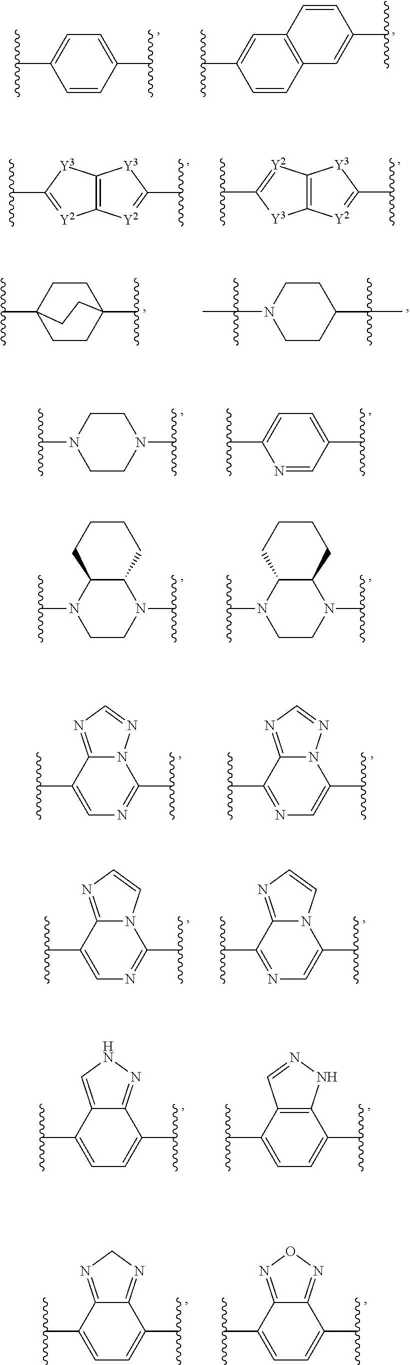 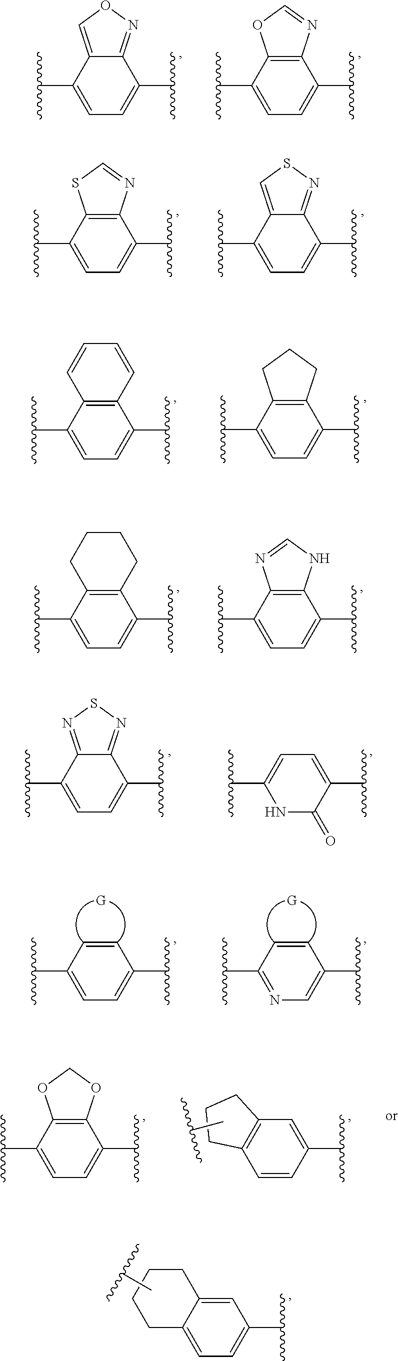
wherein B is unsubstituted or substituted with one or more substituents selected from alkyl, halogen, haloalkyl, hydroxy, alkoxy, haloalkoxy, cyano, or oxo;
or alternatively,
B is selected from 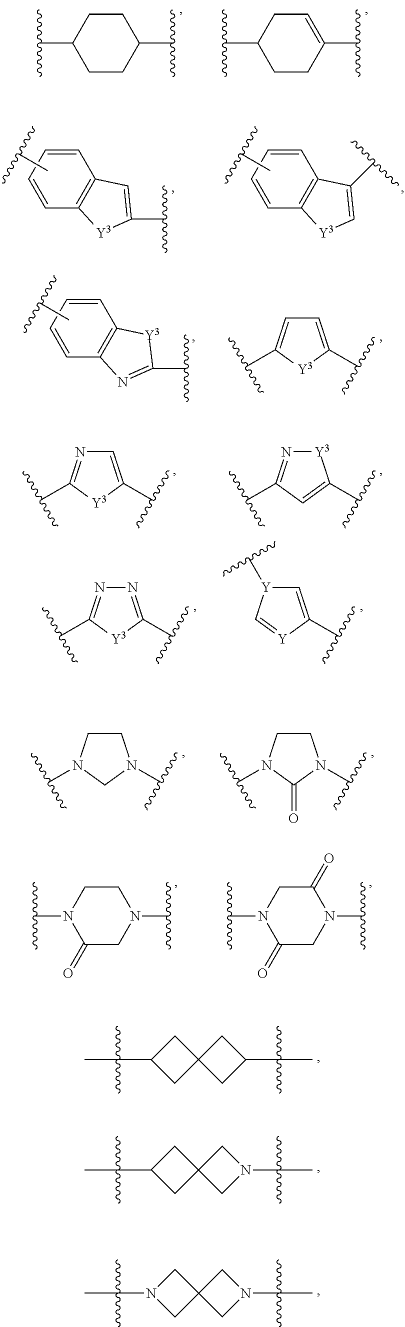 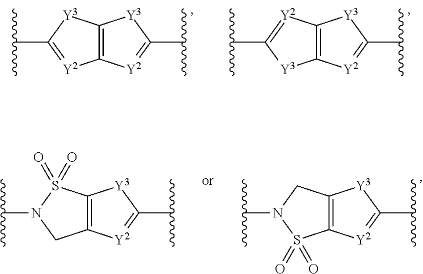
wherein B is unsubstituted or substituted with one or more substituents selected from alkyl, haloalkyl, halogen, hydroxy, alkoxy, haloalkoxy, cyano, or oxo; and
A is selected from 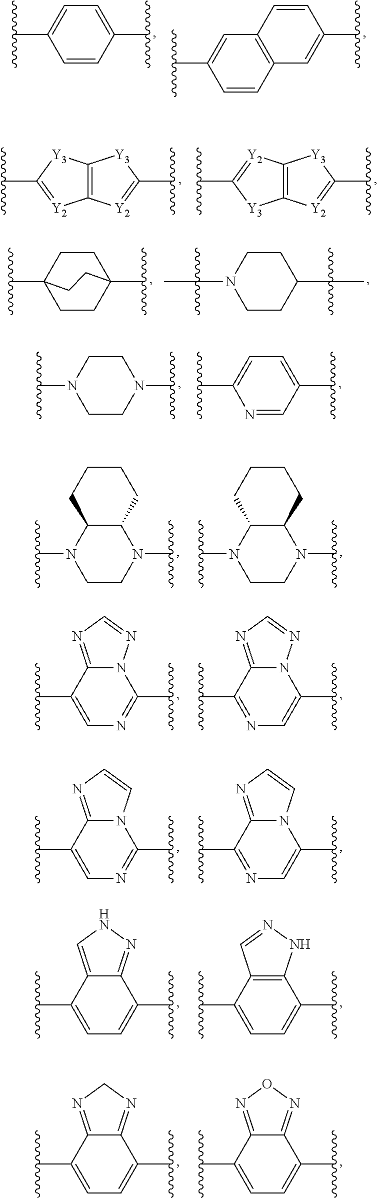 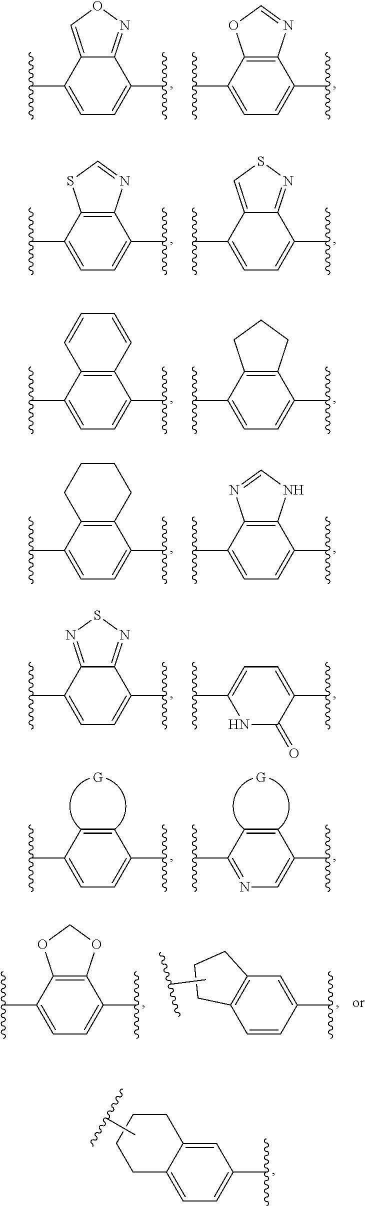
wherein A is unsubstituted or substituted with one or more substituents selected from alkyl, haloalkyl, halogen, hydroxy, alkoxy, haloalkoxy, cyano, or oxo;
G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, or oxo; 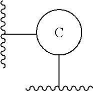
is selected from 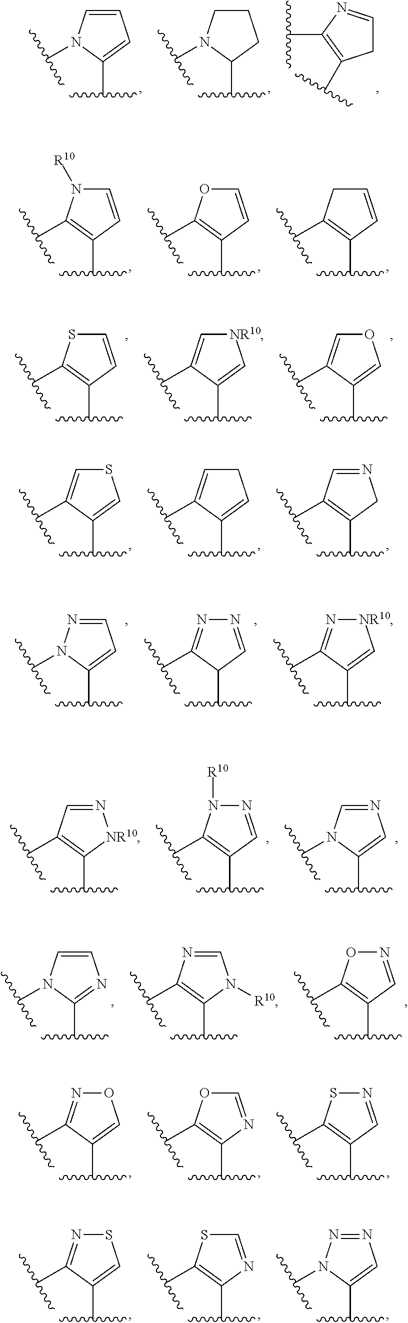 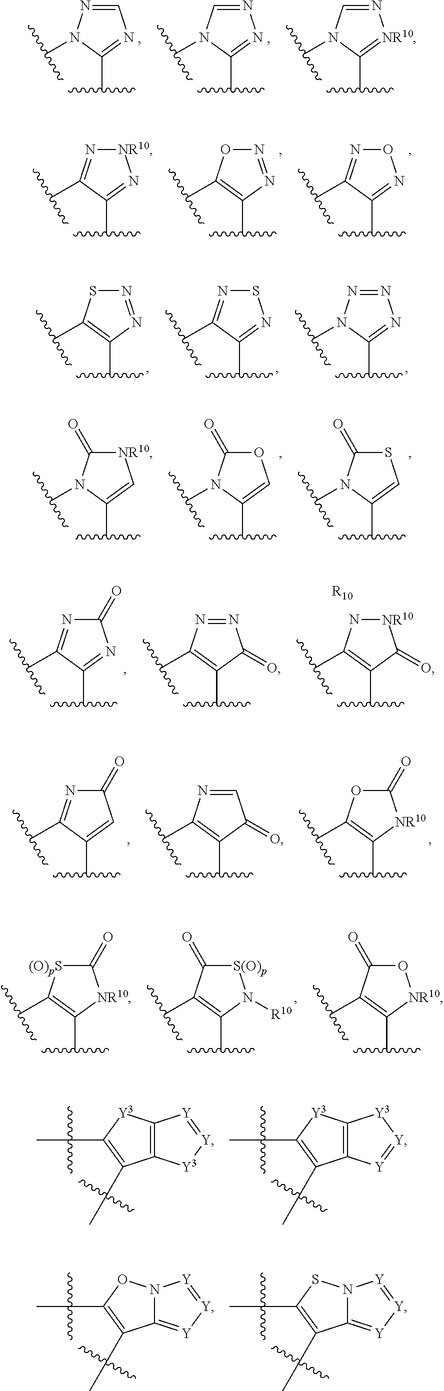 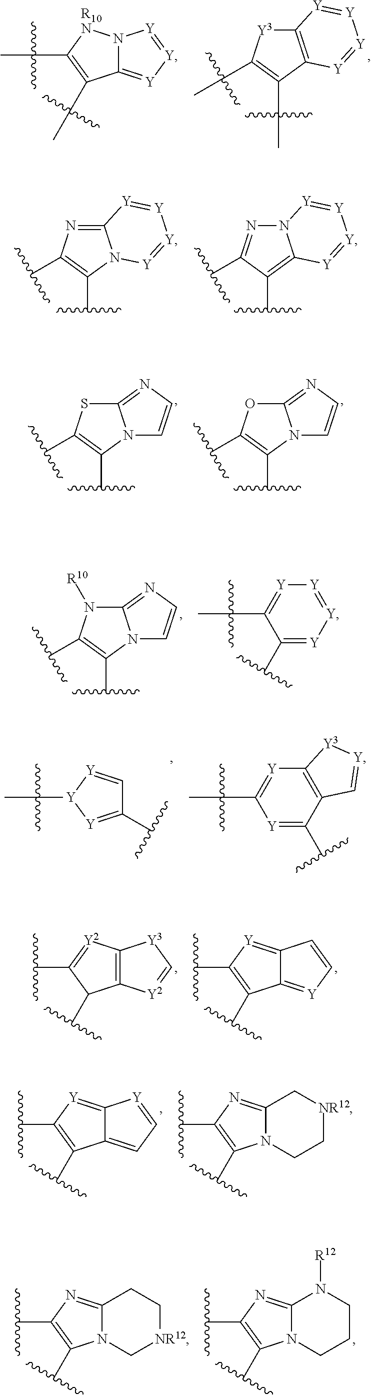 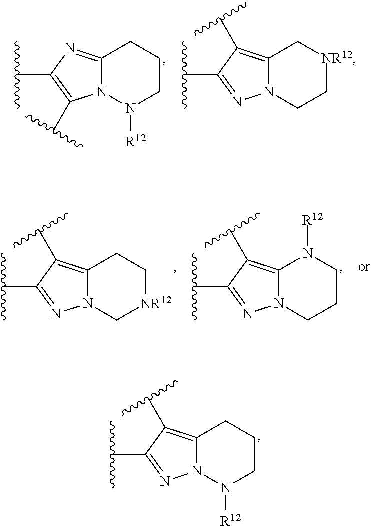
or optionally substituted variants thereof; and
each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido.

72. The compound or pharmaceutically acceptable salt thereof of claim 70, wherein A is selected from 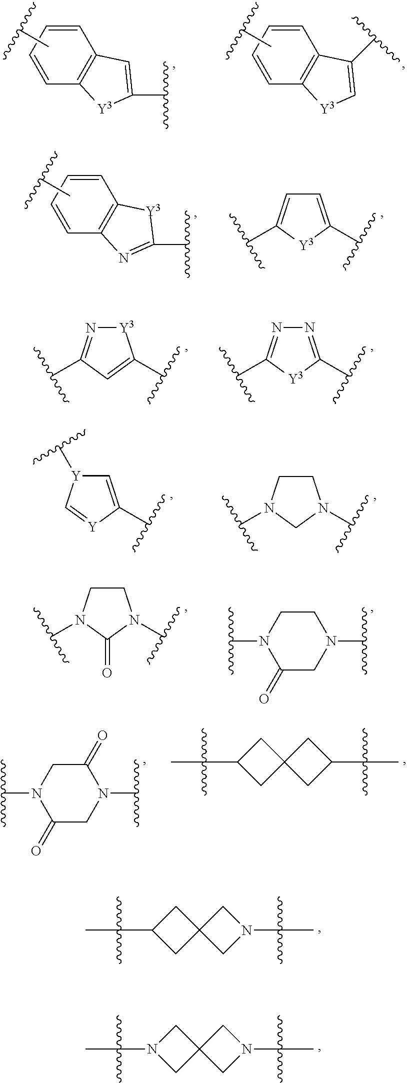 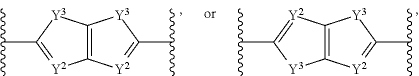
wherein A is unsubstituted or substituted with one or more substituents selected from alkyl, haloalkyl, halogen, hydroxy, alkoxy, haloalkoxy, cyano, or oxo; and
B is selected from 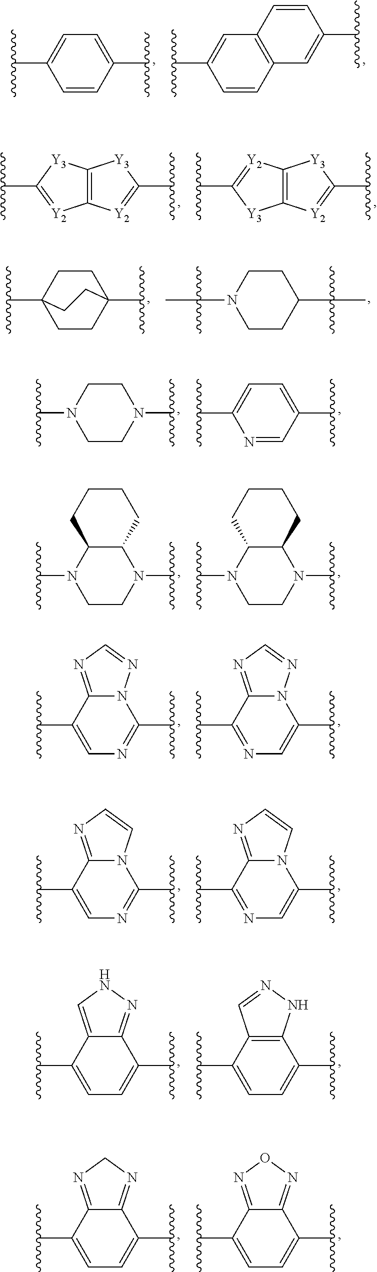 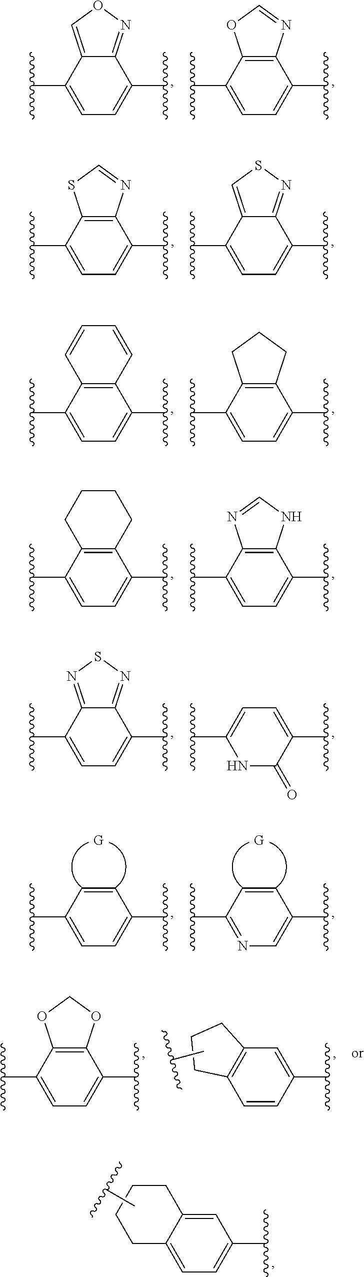
wherein B is unsubstituted or substituted with one or more substituents selected from alkyl, halogen, haloalkyl, hydroxy, alkoxy, haloalkoxy, cyano, or oxo;
or alternatively,
B is selected from 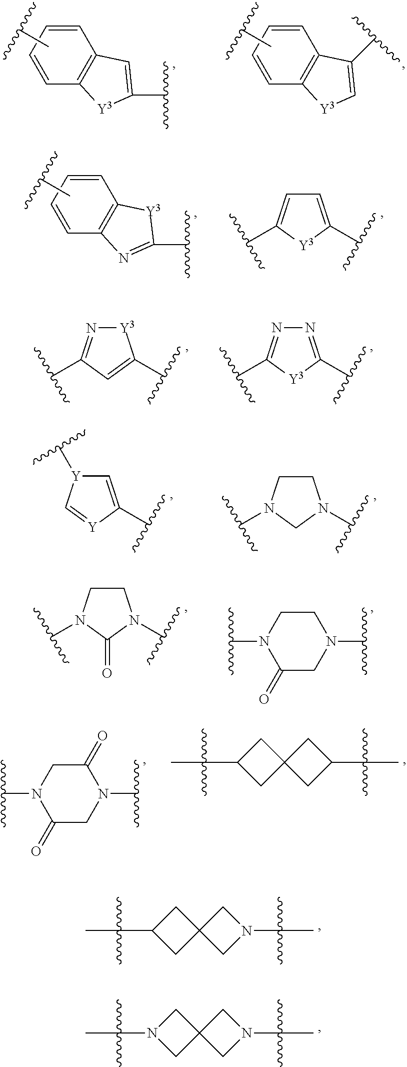 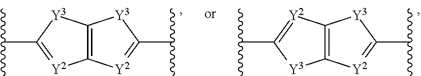
wherein B is unsubstituted or substituted with one or more substituents selected from alkyl, haloalkyl, halogen, hydroxy, alkoxy, haloalkoxy, cyano, or oxo; and
A is selected from 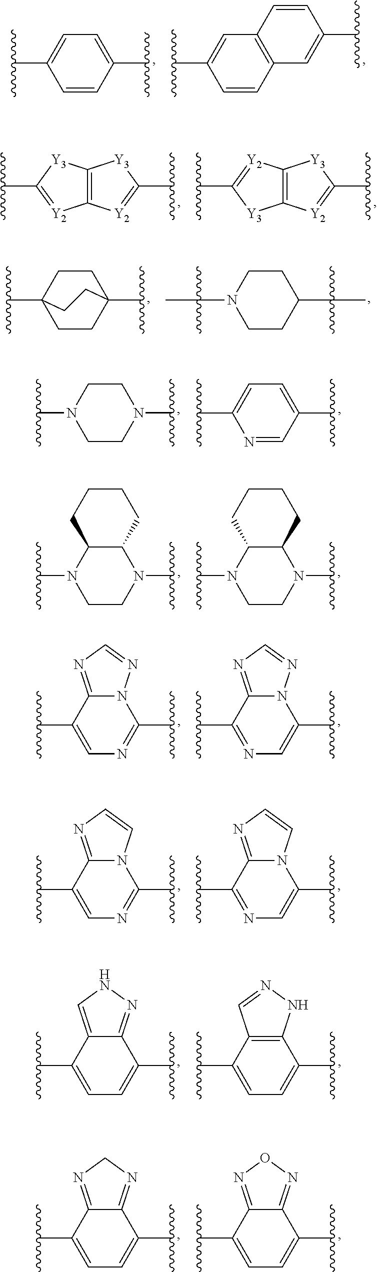 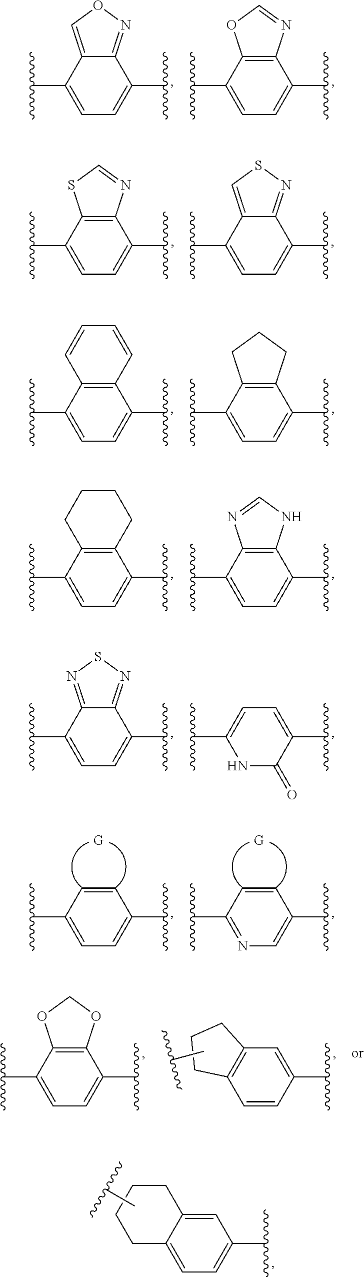
wherein A is unsubstituted or substituted with one or more substituents selected from alkyl, haloalkyl, halogen, hydroxy, alkoxy, haloalkoxy, cyano, or oxo.

73. The compound or pharmaceutically acceptable salt thereof of claim 70, wherein the compound of Formula (III) is also represented by Formula (IIIa): 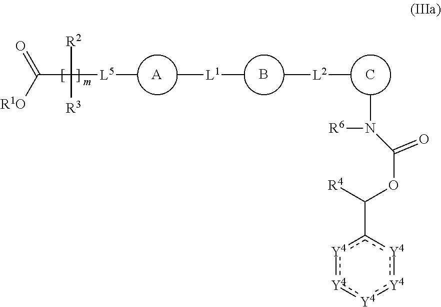
wherein A is selected from 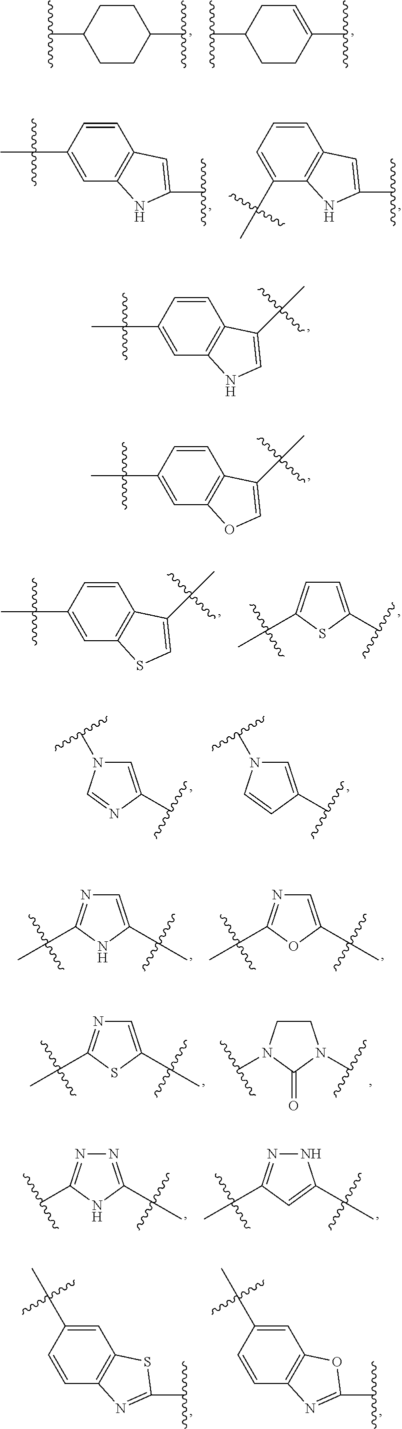 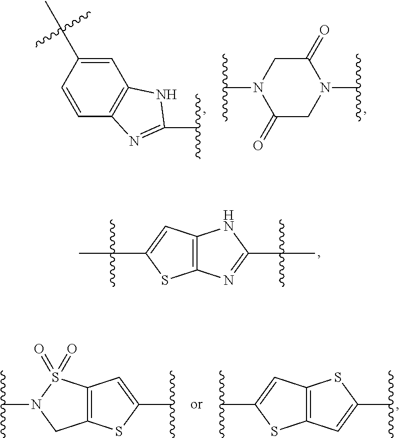 [SMILES: *c1ccc2nc(*)[nH]c2c1 ; *N1CC(=O)N(*)CC1=O ; *c1nc2sc(*)cc2[nH]1 ; *c1cc2c(s1)CN(*)S2(=O)=O ; *c1cc2sc(*)cc2s1]
and wherein A is unsubstituted or substituted with one or more substituents selected from alkyl, haloalkyl, halogen, hydroxy, alkoxy, haloalkoxy, cyano, or oxo;
and B is selected from 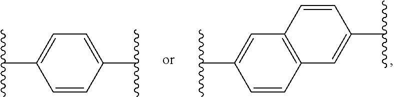 [SMILES: *c1ccc(*)cc1 ; *c1ccc2cc(*)ccc2c1]
each unsubstituted or substituted with one or more substituents selected from alkyl, haloalkyl, halogen, hydroxy, alkoxy, haloalkoxy, cyano, or oxo;
or alternatively,
B is selected from 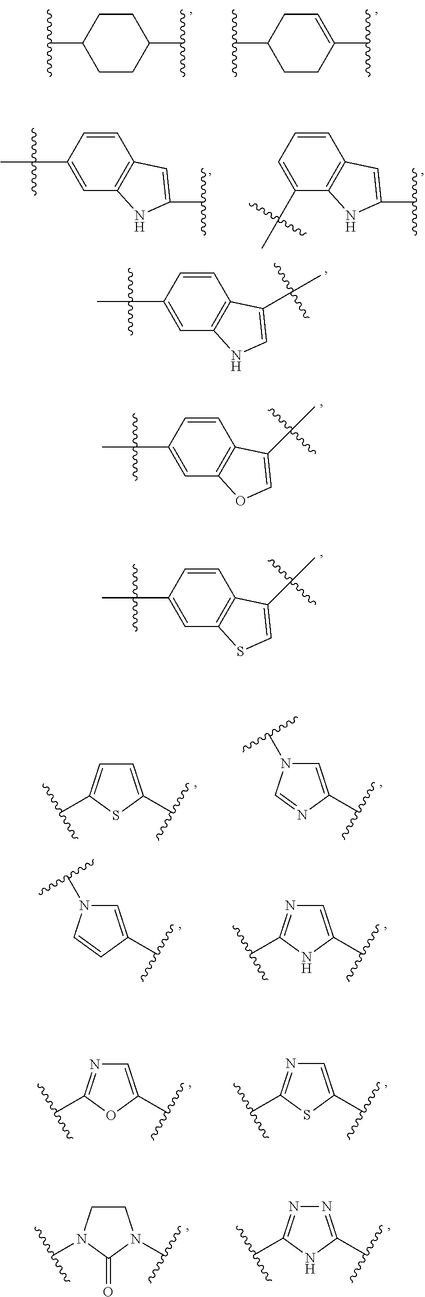 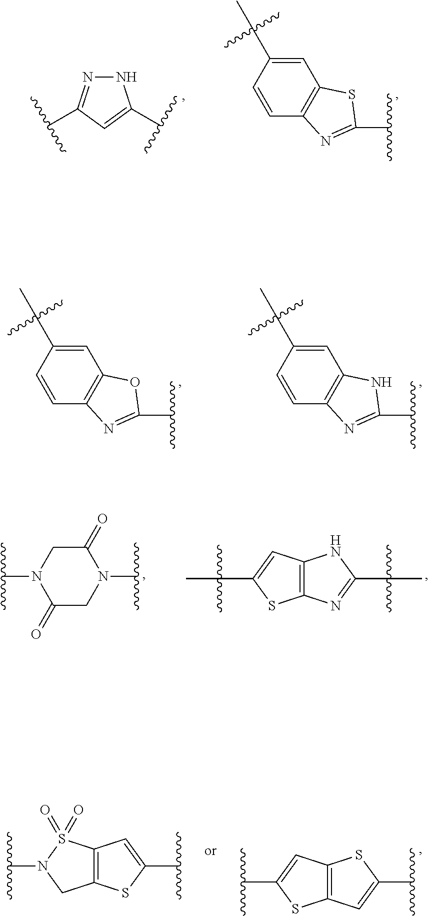
wherein B is unsubstituted or substituted with one or more substituents selected from alkyl, haloalkyl, halogen, hydroxy, alkoxy, haloalkoxy, cyano, or oxo;
and A is selected from 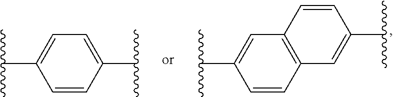 [SMILES: *c1ccc(*)cc1 ; *c1ccc2cc(*)ccc2c1]
each unsubstituted or substituted with one or more substituents selected from alkyl, haloalkyl, halogen, hydroxy, alkoxy, haloalkoxy, cyano, or oxo; and R⁴ is hydrogen or alkyl optionally substituted with halogen.

74. The compound or pharmaceutically acceptable salt thereof of claim 70, wherein
A is selected from 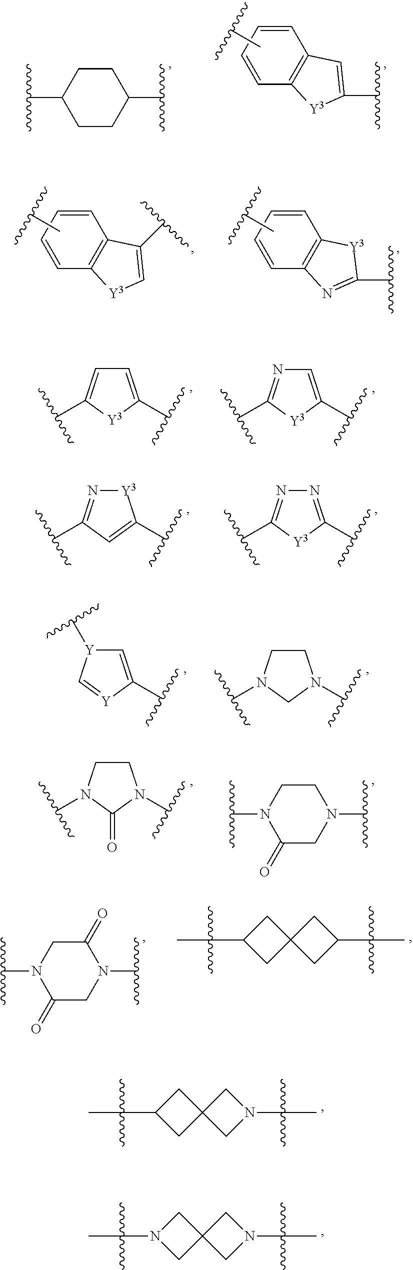 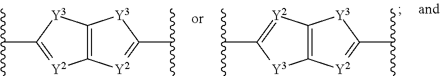
B is a ring system selected from the group consisting of 6-11 membered aryl, 5-11 membered heteroaryl, 4-11 membered heterocyclyl, and 4-11 membered carbocyclyl, and wherein B is unsubstituted or substituted with one or more substituents selected from alkyl, halogen, hydroxy, alkoxy, haloalkoxy, cyano, or oxo;
or alternatively,
B is selected from 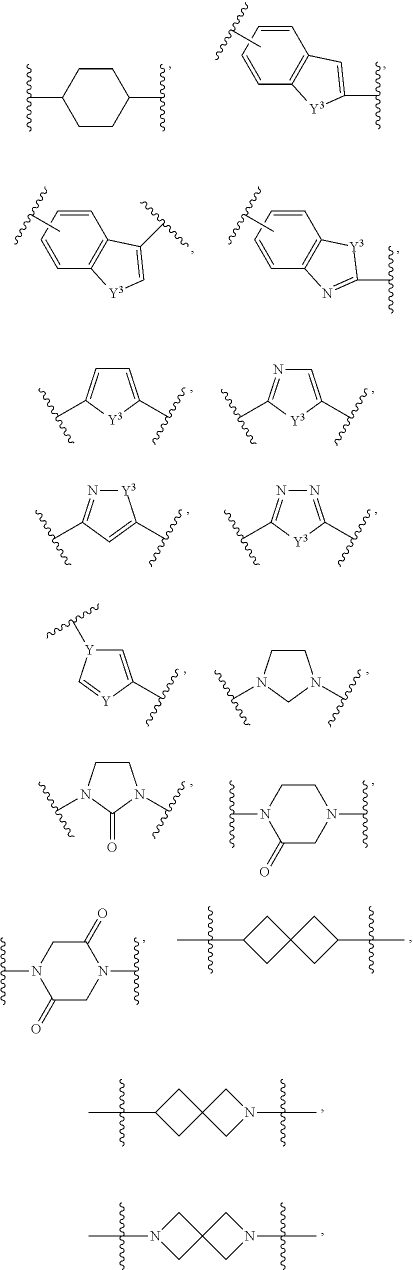 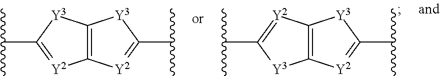
A is a ring system selected from the group consisting of 6-11 membered aryl, 5-11 membered heteroaryl, 4-11 membered heterocyclyl, and 4-11 membered carbocyclyl, and wherein A is unsubstituted or substituted with one or more substituents selected from alkyl, halogen, hydroxy, alkoxy, haloalkoxy, cyano, or oxo;
D is selected from 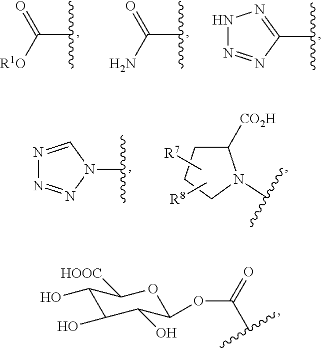
or carboxylic acid isosteres;
R¹ is selected from hydrogen or alkyl;
each R⁴ and R⁵ is independently selected from hydrogen or alkyl; or R⁴ and R⁵ are joined together with the atom to which they are attached to form an optionally substituted cycloalkyl or optionally substituted heterocyclyl;
each R⁶ is independently selected from hydrogen, alkyl, halogen, aryl, or C₃₋₆ cycloalkyl;
each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl;
each R⁹ is independently selected from hydrogen, alkyl or halogen; or two adjacent R⁹ are joined together with the atoms to which they are attached to form an optionally substituted carbocyclyl or an optionally substituted heterocyclyl;
each R¹⁰ is independently selected from hydrogen, alkyl, halogen, aryl, C₃₋₆ cycloalkyl, or cyano; and
each R¹² is independently selected from hydrogen, alkyl, acyl, C-carboxy, C-amido, sulfinyl, sulfonyl, or S-sulfonamido.

75. The compound or pharmaceutically acceptable salt thereof of claim 70, wherein A is a phenyl, unsubstituted or substituted with one or more substituents selected from alkyl, haloalkyl, halogen, hydroxy, alkoxy, haloalkoxy, cyano, or oxo.

76. The compound or pharmaceutically acceptable salt thereof of claim 75, wherein A is substituted with one or more halogen.

77. The compound or pharmaceutically acceptable salt thereof of claim 70, wherein B is a phenyl, unsubstituted or substituted with one or more substituents selected from alkyl, haloalkyl, halogen, hydroxy, alkoxy, haloalkoxy, cyano, or oxo.

78. (canceled)

79. The compound or pharmaceutically acceptable salt thereof of claim 77, wherein B is substituted with one or more halogen.

80. The compound or pharmaceutically acceptable salt thereof of claim 70, wherein C is substituted with one or more substituents selected from C₁₋₃ alkyl optionally substituted with halogen or C₁₋₃ alkoxy; C₁₋₆ alkoxy; C₃₋₆ cycloalkyl; halogen; oxo or cyano.

81. The compound or pharmaceutically acceptable salt thereof of claim 70, wherein C is unsubstituted.

82. The compound or pharmaceutically acceptable salt thereof of claim 70, wherein C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

83. The compound or pharmaceutically acceptable salt thereof of claim 82, wherein C is selected from

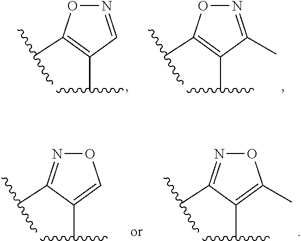

84. The compound or pharmaceutically acceptable salt thereof of claim 82, wherein C is selected from

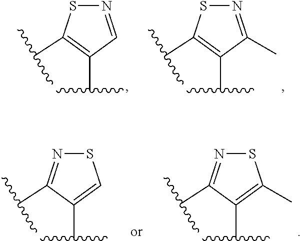

85. The compound or pharmaceutically acceptable salt thereof of claim 70, wherein C is selected from

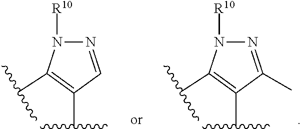

86. (canceled)

87. The compound or pharmaceutically acceptable salt thereof of claim 70, wherein C is

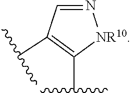

88. The compound or pharmaceutically acceptable salt thereof of claim 70, wherein C is

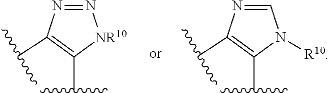

89. (canceled)

90. The compound or pharmaceutically acceptable salt thereof of claim 70, wherein C is selected from

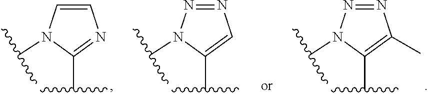

91. (canceled)

92. (canceled)

93. (canceled)

94. (canceled)

95. The compound or pharmaceutically acceptable salt thereof of claim 70, wherein m is 0.

96. The compound or pharmaceutically acceptable salt thereof of claim 70, wherein m is 1.

97. The compound or pharmaceutically acceptable salt thereof of claim 70, wherein each of R² and R³ is hydrogen.

98. The compound or pharmaceutically acceptable salt thereof of claim 70, wherein R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl.

99. The compound or pharmaceutically acceptable salt thereof of claim 98, wherein R² and R³ are joined together with the atom to which they are attached to form an optionally substituted cyclopropyl.

100. The compound or pharmaceutically acceptable salt thereof of claim 70, wherein L⁵ is a single bond.

101. The compound or pharmaceutically acceptable salt thereof of claim 70, wherein L² is a single bond.

102. The compound or pharmaceutically acceptable salt thereof of claim 70, wherein L¹ is a single bond.

103. (canceled)

104. The compound or pharmaceutically acceptable salt thereof of claim 70, wherein L¹ is a 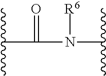
linker.

105. The compound or pharmaceutically acceptable salt thereof of claim 70, wherein R⁶ is hydrogen.

106. The compound or pharmaceutically acceptable salt thereof of claim 70, wherein R¹ is hydrogen.

107. The compound or pharmaceutically acceptable salt thereof of claim 70, wherein R⁴ is alkyl.

108. The compound or pharmaceutically acceptable salt thereof of claim 70, wherein

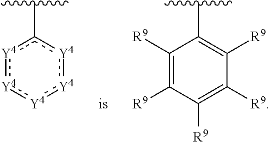

109. (canceled)

110. (canceled)

111. The compound or pharmaceutically acceptable salt thereof of claim 70, wherein 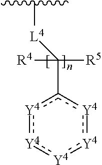
is selected from

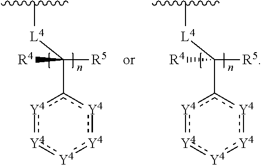

112. The compound or pharmaceutically acceptable salt thereof of claim 70, selected from compounds of Table 3, and pharmaceutically acceptable salt thereof.

113. The compound or pharmaceutically acceptable salt thereof of claim 70, selected from compounds IT007-IT010, IT025, IT046, IT050, IT051, IT053, IT054, IT056, IT059, IT060, IT066, IT067, IT071 or IT091 of Table 12.

114-223. (canceled)

224. A compound of Formula (VII): 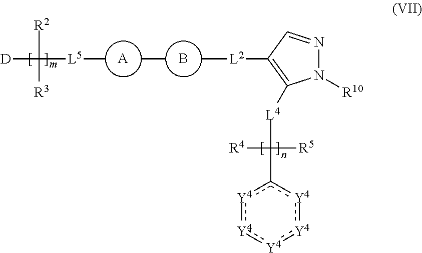
or a pharmaceutically acceptable salt thereof, wherein:
A is an acetylene and B is a ring system selected from the group consisting of 6-11 membered aryl, 5-11 membered heteroaryl, 4-11 membered heterocyclyl, and 4-11 membered carbocyclyl, wherein B is unsubstituted or substituted with one or more substituents selected from alkyl, halogen, haloalkyl, hydroxy, alkoxy, haloalkoxy, cyano, or oxo;
or alternatively,
B is an acetylene, or is absent when L² is —(CH₂)ₖ— linker, and A is a ring system selected from the group consisting of 6-11 membered aryl, 5-11 membered heteroaryl, 4-11 membered heterocyclyl, and 4-11 membered carbocyclyl, wherein A is unsubstituted or substituted with one or more substituents selected from alkyl, halogen, haloalkyl, hydroxy, alkoxy, haloalkoxy, cyano, or oxo; or B is optionally absent when L² is —(CH₂)ₖ— linker;
D is selected from —OH, 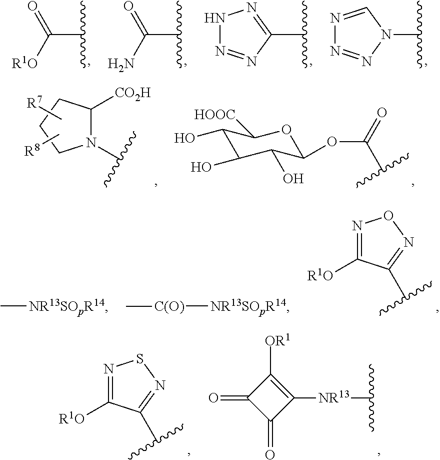
or carboxylic acid isosteres; 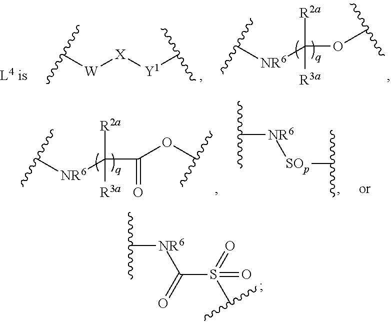
L² is selected from a single bond, a —O— linker, a 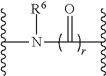
linker, a —C(O)— linker, a —CH₂— linker, a —(CH₂)ₖ— linker, a —CH₂O— linker, a —C≡C— linker, or a —CH═CH— linker;
L⁵ is selected from a single bond, a —CH₂O— linker, a —CH═CH— linker, a —C≡C— linker, a 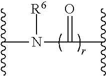
linker, or a 4-7 membered heterocyclyl;
W is selected from C(R⁶)₂, NR⁶, or O;
X is selected from —C(O) or S(O)ₚ;
Y¹ is selected from C(R⁶)₂, NR⁶, or O;
each Y⁴ is independently absent, CR⁹, C(R⁹)₂, N, or NH, provided that only one Y⁴ can be absent;
R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;
R² and R³ are each independently selected from hydrogen, alkyl, aryl, or heteroaryl; or R² and R³ are joined together with the atom to which they are attached to form an optionally substituted cycloalkyl or an optionally substituted heterocyclyl;
or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;
each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;
each R⁶ is independently selected from hydrogen; alkyl optionally substituted with one or more substituents selected from the group consisting of halogen, hydroxy and alkoxy; halogen; aryl; or C₃₋₆ cycloalkyl;
each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;
each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;
each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;
each R¹³ is independently selected from hydrogen, alkyl, haloalkyl, aryl, or C₃₋₆ cycloalkyl;
each R¹⁴ is independently selected from hydrogen, alkyl, haloalkyl, aryl, or C₃₋₆ cycloalkyl;
each R^{2a} and R^{3a} is independently selected from hydrogen, alkyl, aryl, or heteroaryl; or R^{2a} and R^{3a} are joined together with the atom to which they are attached to form an optionally substituted cycloalkyl or an optionally substituted heterocyclyl;
m is independently an integer from 0-3;
n is an integer from 0-3;
k is an integer from 2-4;
p is an integer from 1-2;
q is an integer from 1-6;
r is an integer of 0 or 1, and [symbol]
represents a single or double bond.

225. The compound or pharmaceutically acceptable salt thereof of claim 224, wherein
A is acetylene and B is selected from the group consisting of 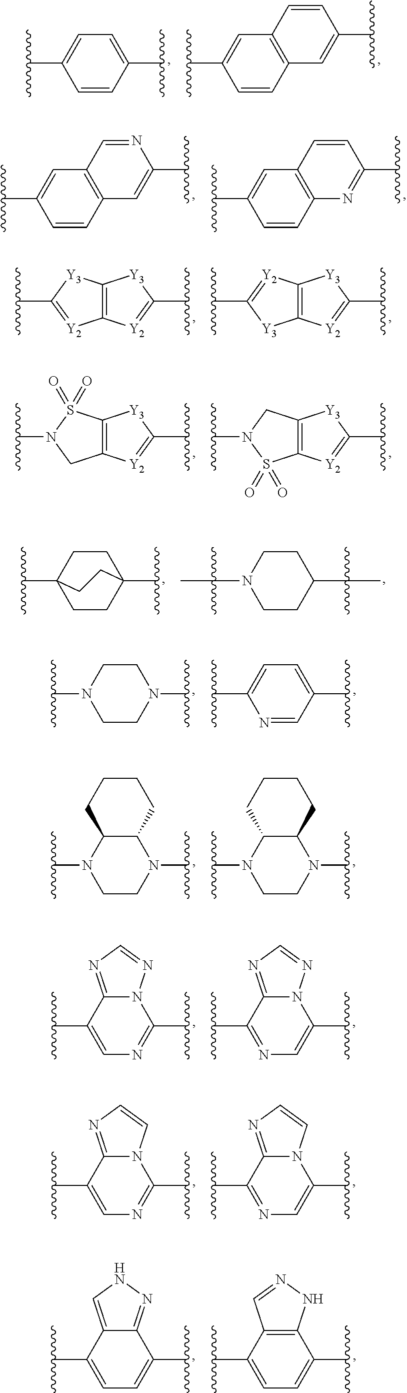 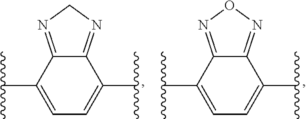 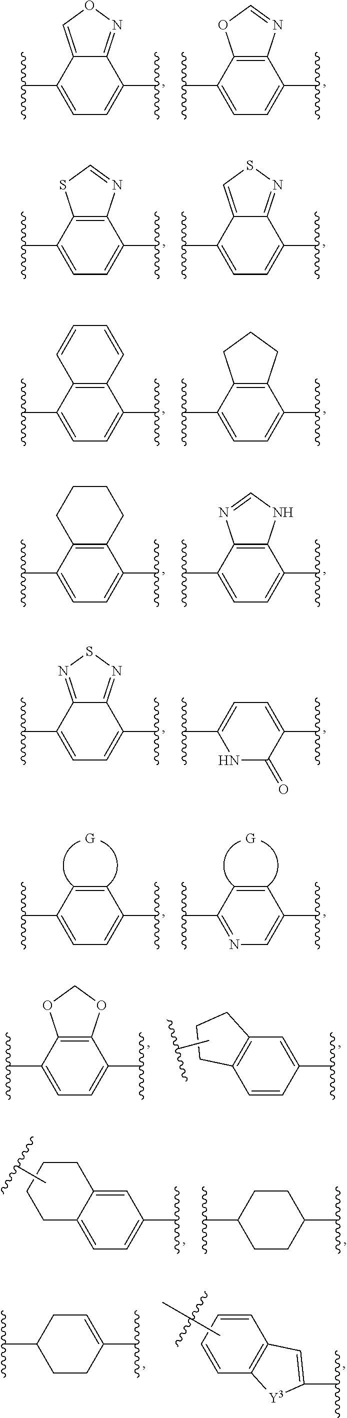 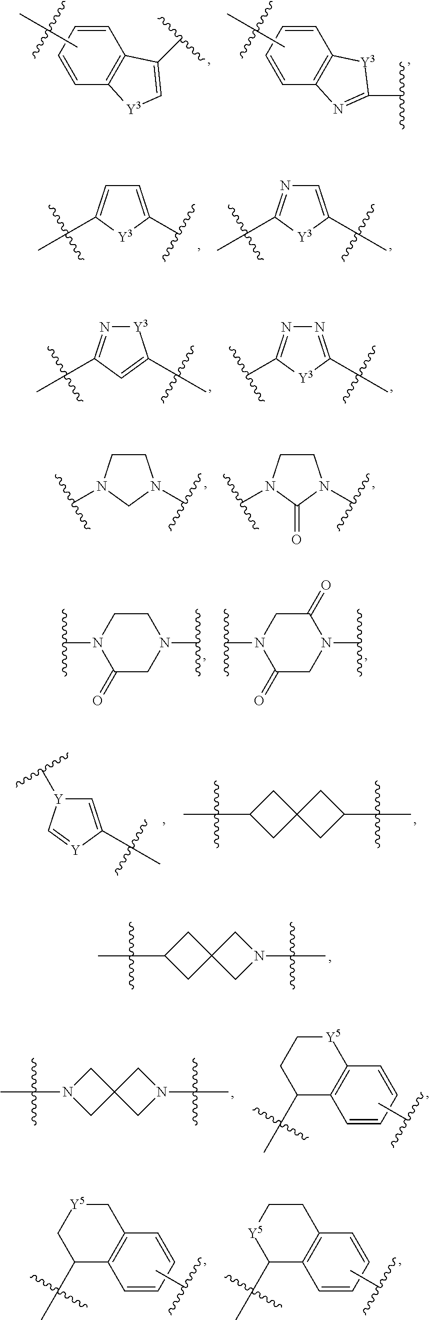 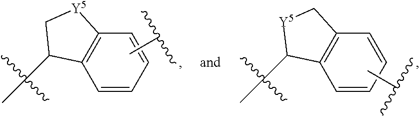
wherein the rings in B are unsubstituted or substituted with one or more substituents selected from alkyl, haloalkyl, halogen, hydroxy, alkoxy, haloalkoxy, cyano, or oxo;
or alternatively,
B is an acetylene and A is selected from the group consisting of 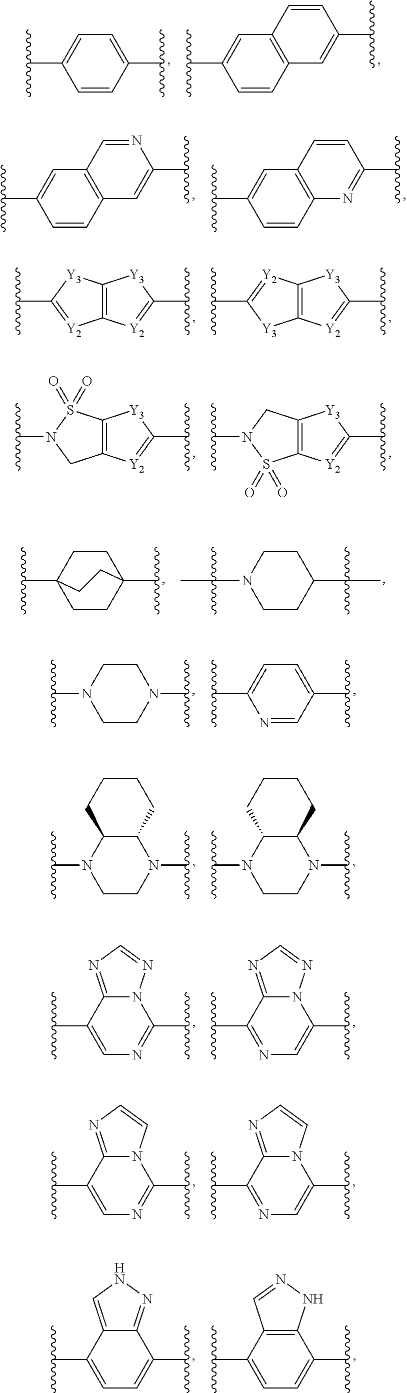 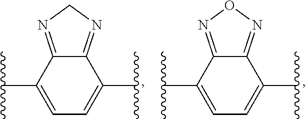 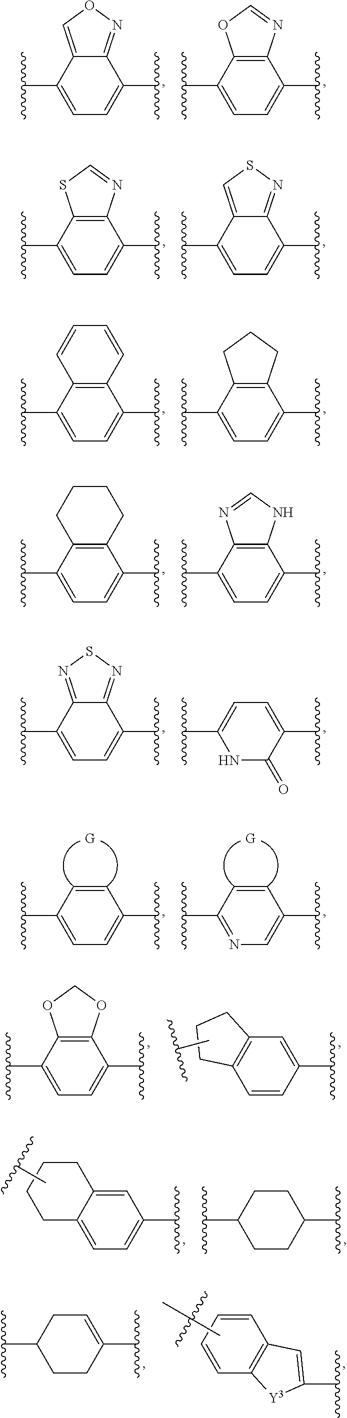 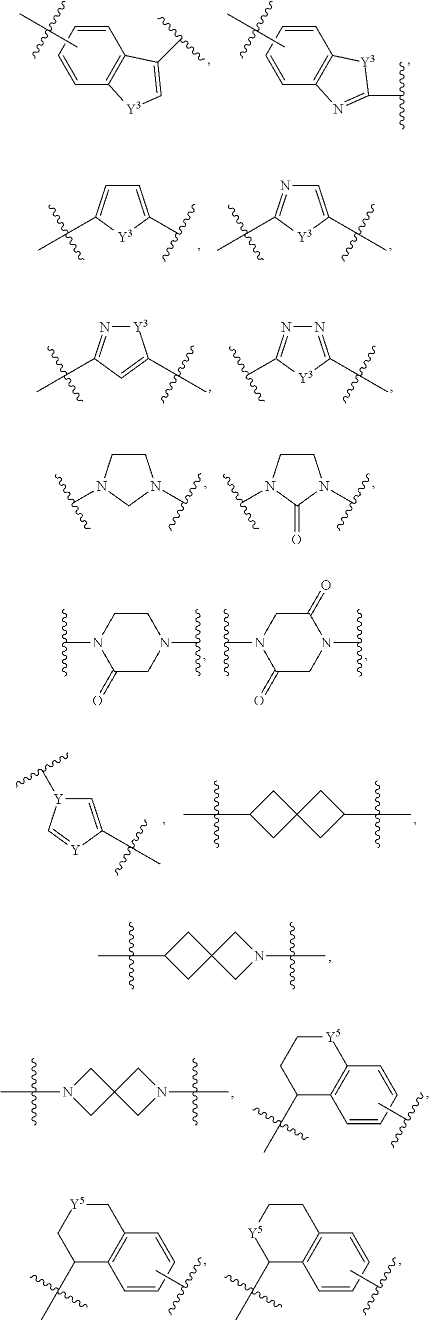 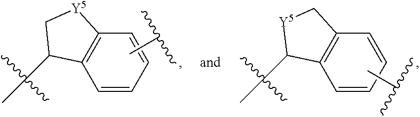
wherein the rings in A are unsubstituted or substituted with one or more substituents selected from alkyl, haloalkyl, halogen, hydroxy, alkoxy, haloalkoxy, cyano, or oxo;
G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, haloalkyl, halogen, hydroxy, alkoxy, haloalkoxy, cyano, or oxo;
each Y is independently selected from CR⁶ or N;
Y² is selected from —CH═ or N;
Y³ is selected from C(R⁶)₂, NR⁶, O or S;
Y⁵ is selected from NR⁶, O or S; and
D is selected from —OH, 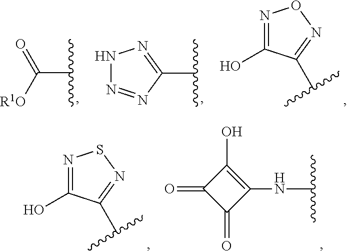
—NHS(O)₂R¹⁴, or —C(O)—NHS(O)₂R¹⁴.

226. The compound or pharmaceutically acceptable salt thereof of claim 224, wherein the compound of Formula (VII) is also represented by Formula (VIIa): 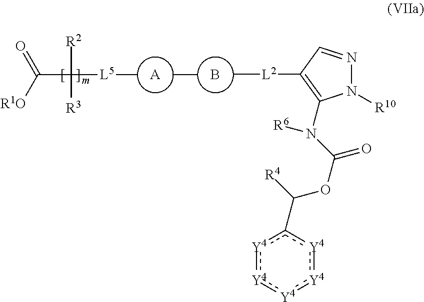
wherein A is an acetylene and B is selected from 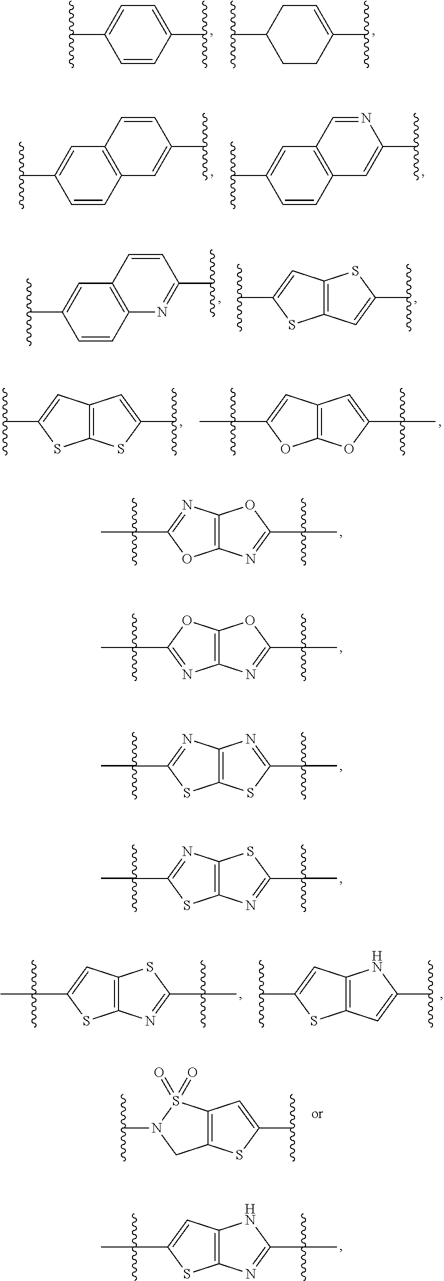
and wherein the rings in B are unsubstituted or substituted with one or more substituents selected from alkyl, haloalkyl, halogen, hydroxy, alkoxy, haloalkoxy, cyano, or oxo;
or alternatively,
B is an acetylene and A is selected from 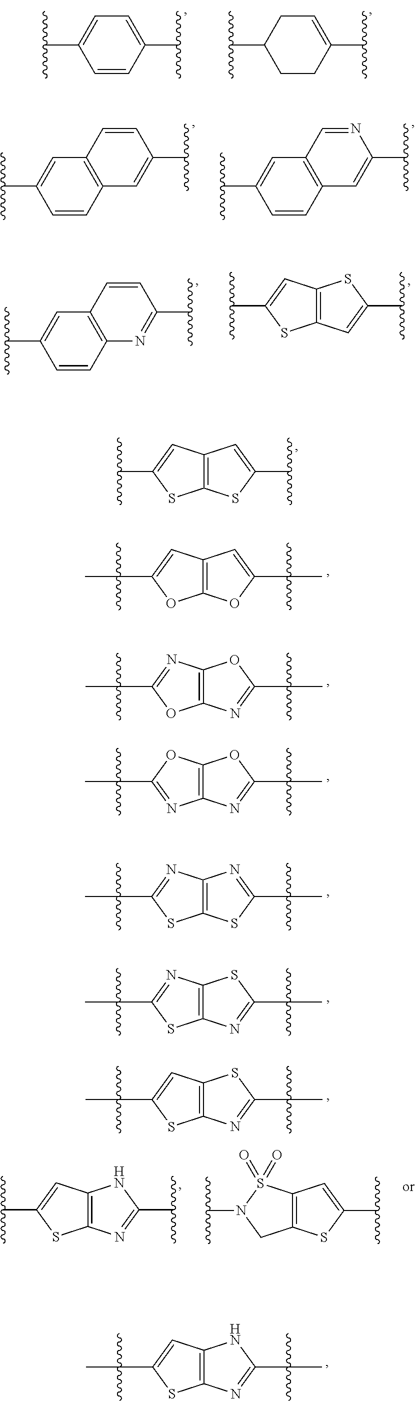
and wherein the rings in A are unsubstituted or substituted with one or more substituents selected from alkyl, haloalkyl, halogen, hydroxy, alkoxy, haloalkoxy, cyano, or oxo.

227. The compound or pharmaceutically acceptable salt thereof of claim 224, wherein
D is selected from 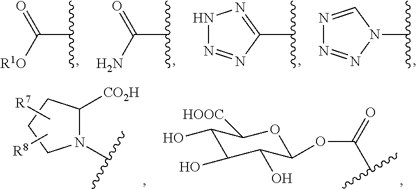
or carboxylic acid isosteres;
L² is selected from a single bond, a —O— linker, a 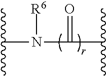
linker, a —C(O)— linker, a —CH₂— linker, a —CH₂O— linker, a —C≡C— linker, or a —CH═CH— linker;
R¹ is selected from hydrogen or alkyl;
each R⁴ and R⁵ is independently selected from hydrogen or alkyl; or R⁴ and R⁵ are joined together with the atom to which they are attached to form an optionally substituted cycloalkyl or optionally substituted heterocyclyl;
each R⁶ is independently selected from hydrogen, alkyl, halogen, aryl, or C₃₋₆ cycloalkyl;
each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl;
each R⁹ is independently selected from hydrogen, alkyl or halogen; or two adjacent R⁹ are joined together with the atoms to which they are attached to form an optionally substituted carbocyclyl or an optionally substituted heterocyclyl; and
each R¹⁰ is independently selected from hydrogen, alkyl, halogen, aryl, C₃₋₆ cycloalkyl, or cyano.

228. The compound or pharmaceutically acceptable salt thereof of claim 224, wherein A is acetylene and B is phenyl.

229. (canceled)

230. The compound or pharmaceutically acceptable salt thereof of claim 224, wherein A is acetylene and B is selected from 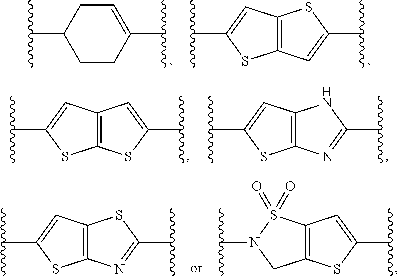
each optionally substituted with one or more substituents selected from the group consisting of alkyl, alkoxy, halogen, haloalkyl and cyano.

231. The compound or pharmaceutically acceptable salt thereof of claim 224, wherein B is acetylene and A is phenyl.

232. (canceled)

233. The compound or pharmaceutically acceptable salt thereof of claim 224, wherein B is acetylene and A is selected from 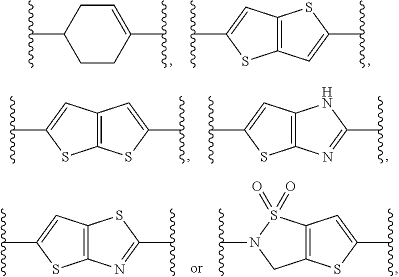
each optionally substituted with one or more substituents selected from the group consisting of alkyl, alkoxy, halogen and cyano.

234. (canceled)

235. The compound or pharmaceutically acceptable salt thereof of claim 224, wherein R¹⁰ is C₁₋₃ alkyl.

236. The compound or pharmaceutically acceptable salt thereof of claim 224, wherein R¹⁰ is C₃₋₆ cycloalkyl.

237. The compound or pharmaceutically acceptable salt thereof of claim 224, wherein R¹ is hydrogen or unsubstituted alkyl.

238. The compound or pharmaceutically acceptable salt thereof of claim 224, wherein R¹ is alkyl substituted with one or more substituents selected from the group consisting of alkoxy, C-amido, O-carboxy, and 6 membered heterocyclyl.

239. The compound or pharmaceutically acceptable salt thereof of claim 224, wherein R¹ is optionally substituted aryl.

240. The compound or pharmaceutically acceptable salt thereof of claim 224, wherein m is 0.

241. The compound or pharmaceutically acceptable salt thereof of claim 224, wherein m is 1.

242. The compound or pharmaceutically acceptable salt thereof of claim 224, wherein m is 2.

243. The compound or pharmaceutically acceptable salt thereof of claim 224, wherein each of R² and R³ is hydrogen.

244. The compound or pharmaceutically acceptable salt thereof of claim 224, wherein one of R² and R³ is hydrogen and the other R² and R³ is aryl.

245. The compound or pharmaceutically acceptable salt thereof of claim 224, wherein R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl.

246. The compound or pharmaceutically acceptable salt thereof of claim 245, wherein R² and R³ are joined together with the atom to which they are attached to form an optionally substituted cyclopropyl.

247. The compound or pharmaceutically acceptable salt thereof of claim 224, wherein R⁶ is hydrogen.

248. (canceled)

249. The compound or pharmaceutically acceptable salt thereof of claim 224, wherein L⁵ is a single bond.

250. The compound or pharmaceutically acceptable salt thereof of claim 224, wherein L² is a single bond.

251. (canceled)

252. (canceled)

253. The compound or pharmaceutically acceptable salt thereof of claim 224, wherein

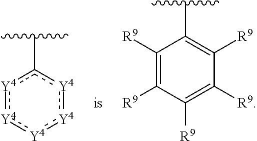

254. (canceled)

255. (canceled)

256. The compound or pharmaceutically acceptable salt thereof of claim 224, wherein 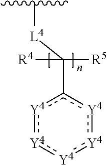
is selected from

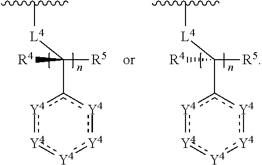

257. The compound or pharmaceutically acceptable salt thereof of claim 224, selected from compounds of Table 7.

258. The compound or pharmaceutically acceptable salt thereof of claim 224, selected from compounds IT014-IT018, IT070, IT082-IT090, IT092, IT095, IT097-IT100 or IT102 of Table 12.

259-381. (canceled)

382. A compound or pharmaceutically acceptable salt thereof, selected from compounds IT004, IT026-036, IT038-IT045, IT047-IT049, IT052, IT055, IT061, IT064, IT068, IT069, IT072-IT081, IT093, IT094, IT096 and IT102.

383. A pharmaceutical composition comprising an effective amount of a compound of claim 70, or a pharmaceutically acceptable salt thereof, and a pharmaceutically acceptable carrier, diluent, excipient or combination thereof.

384. A method for treating, preventing, reversing, halting, or slowing the progression of a disease or condition selected from fibrosis, cancer, or respiratory disorders, comprising administering an effective amount of a compound of claim 70, or a pharmaceutically acceptable salt thereof, or a pharmaceutical composition to a subject in need thereof.

385. The method of claim 384, wherein the disease or condition is fibrosis.

386. The method of claim 384, wherein the fibrosis is selected from pulmonary fibrosis, dermal fibrosis, kidney fibrosis, or liver fibrosis.

387. The method of claim 384, wherein the fibrosis is idiopathic pulmonary fibrosis.

388. The method of claim 384, wherein the respiratory disorders is selected from asthma, COPD, or rhinitis.

389. A method of modulating a LPA receptor activity in a cell comprising contacting the cell with an effective amount of a compound of claim 70, or a pharmaceutically acceptable salt thereof.

390. The method of claim 389, wherein the LPA receptor is LPA₁.

391-441. (canceled)

442. A pharmaceutical composition comprising an effective amount of a compound of claim 224, or a pharmaceutically acceptable salt thereof, and a pharmaceutically acceptable carrier, diluent, excipient or combination thereof.

443. A method for treating, preventing, reversing, halting, or slowing the progression of a disease or condition selected from fibrosis, cancer, or respiratory disorders, comprising administering an effective amount of a compound of claim 224, or a pharmaceutically acceptable salt thereof to a subject in need thereof.

444. The method of claim 443, wherein the disease or condition is fibrosis.

445. The method of claim 443, wherein the fibrosis is selected from pulmonary fibrosis, dermal fibrosis, kidney fibrosis, or liver fibrosis.

446. The method of claim 443, wherein the fibrosis is idiopathic pulmonary fibrosis.

447. The method of claim 443, wherein the respiratory disorders is selected from asthma, COPD, or rhinitis.

448. A method of modulating a LPA receptor activity in a cell comprising contacting the cell with an effective amount of a compound of claim 224, or a pharmaceutically acceptable salt thereof.

449. The method of claim 448, wherein the LPA receptor is LPA₁.
